(19) United States
(12) Patent Application Publication
(10) Pub. No.: US 2026/0365646 A1
(43) Pub. Date: Nov. 19, 2026

(54) AKT1 MODULATORS

(71) Applicant: Alterome Therapeutics, Inc., San Diego, CA (US)

(72) Inventors: Michael David BARTBERGER, Sherman Oaks, CA (US); Elena V. DNEPROVSKAIA, San Diego, CA (US); Yi FAN, San Diego, CA (US); Eric Anthony MURPHY, San Marcos, CA (US); Xuefeng ZHU, San Diego, CA (US)

(21) Appl. No.: 19/106,008

(22) PCT Filed: Sep. 6, 2023
(86) PCT No.: PCT/US2023/032104

§ 371 Date: Feb. 24, 2025

Related U.S. Application Data
(60) Provisional application No. 63/375,023, filed on Sep. 8, 2022.

(51) Int. Cl. C07D 471/04; A61K 31/444; A61K 31/4545; A61K 31/496; A61K 31/499; A61K 31/55; A61K 31/551; A61K 31/553; A61K 31/554; C07B 59/00; C07D 519/00

(52) U.S. Cl. CPC C07D 471/04; A61K 31/444; A61K 31/4545; A61K 31/496; A61K 31/499; A61K 31/55; A61K 31/551; A61K 31/553; A61K 31/554; C07B 59/002; C07D 519/00

(57) ABSTRACT

Provided herein are inhibitors of AKT1, pharmaceutical compositions comprising the inhibitory compounds, and methods for using the AKT1 inhibitory compounds for the treatment of disease.

### CROSS-REFERENCE

This application claims the benefit of U.S. Provisional Application No. 63/375,023 filed Sep. 8, 2022, which is incorporated herein by reference in its entirety.

### BACKGROUND

AKT is a protein kinase and mediates cell survival and proliferation by inhibiting pathways which promotes apoptosis. AKT signaling cascade dysfunction is observed in several cancer types and may be associated with tumor aggressiveness. Additionally, malfunction of AKT typically lead to enhanced proliferation, growth, survival, and resistance to apoptosis.

Pharmaceutical agents with the ability to modulate AKT1 activity would be useful in the treatment of disease, such as cancer.

### BRIEF SUMMARY OF THE INVENTION

Provided herein are inhibitors of AKT1, pharmaceutical compositions comprising said inhibitory compounds, and methods for using said inhibitory compounds for the treatment of disease.

One embodiment provides a compound having the structure of Formula (I), or a pharmaceutically acceptable salt or solvate thereof:

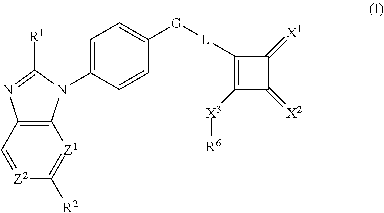

wherein:
G is O or CR⁴R⁵;
Z¹ is N, C—H, or C—R⁹;
Z² is N, C—H, or C—R³;
X¹ is O or S;
X² is O or S;
X³ is a bond, O, S, N—R⁷;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ and R⁵ are each independently hydrogen, halogen, —OH, or optionally substituted C1-C6 alkyl; or R⁴ and R⁵ together form an oxo; or R⁴ and R⁵ join together to form a carbocycle or heterocycle;
R⁶ is hydrogen, optionally substituted C1-C6 alkyl, optionally substituted C3-C7 cycloalkyl, and optionally substituted heterocyclyl; or R⁶ is absent and X³ and L join together to form a heterocycle;
R⁷ is selected from hydrogen, —OH, —NH₂, optionally substituted C1-C6 alkyl, optionally substituted C3-C7 cycloalkyl, and optionally substituted heterocyclyl; or optionally, R⁶ and R⁷ join together to form a heterocycle;
L is selected from —N(R⁸)—, or a divalent radical selected from:

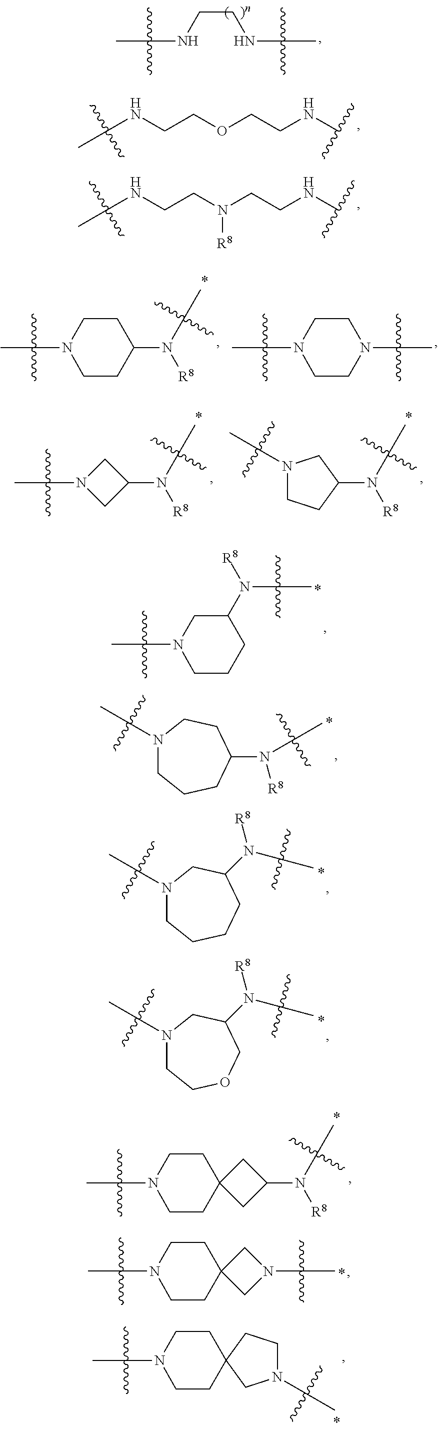

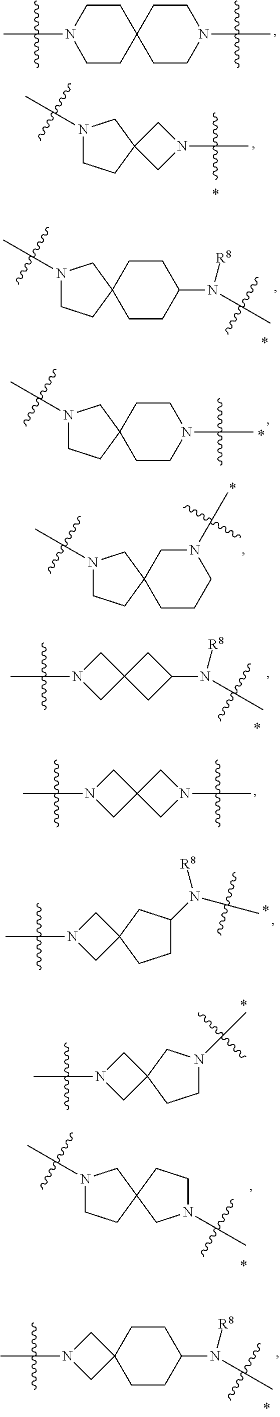

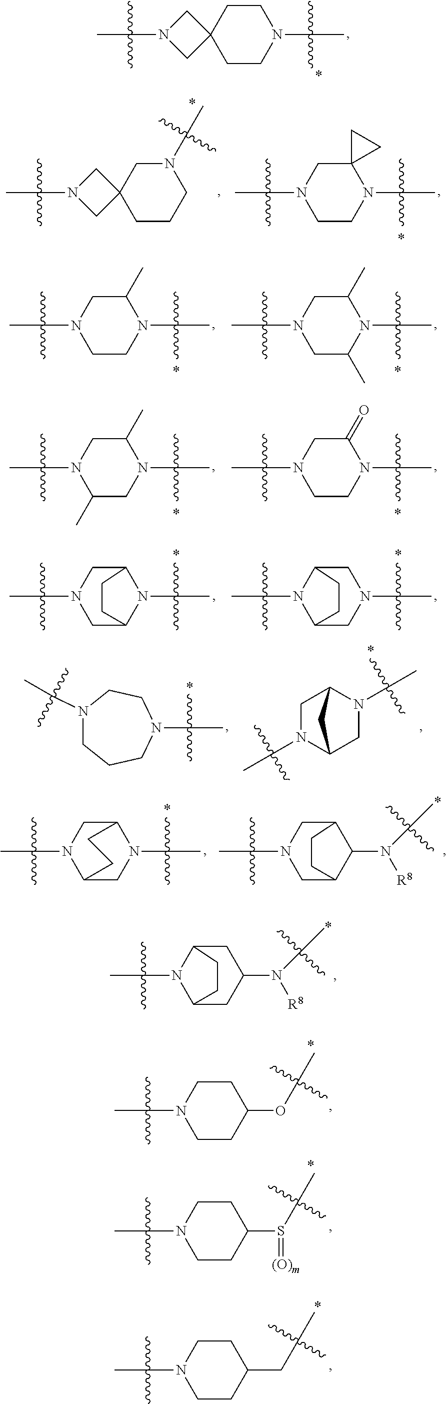

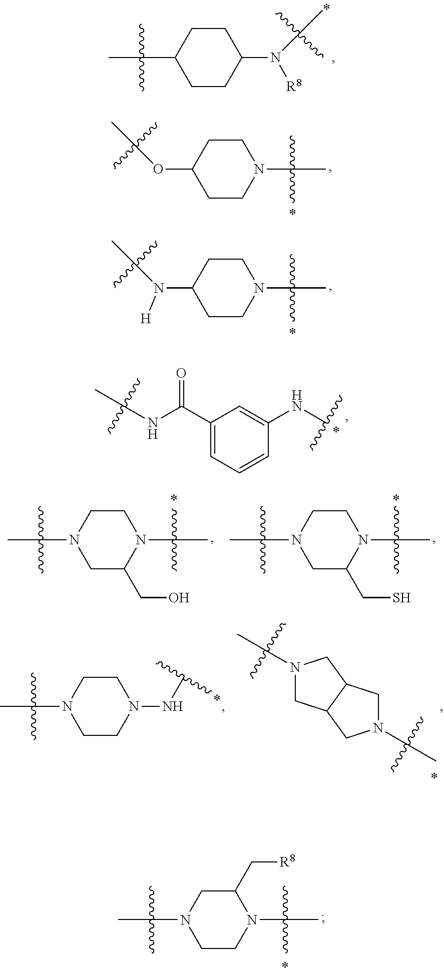

wherein the asterisk (*) indicates the bond to the squaric acid group;
R⁸ is hydrogen, optionally substituted C1-C6 alkyl, optionally substituted C3-C7 cycloalkyl, and optionally substituted heterocyclyl; or optionally R⁸ and R⁶ join to form a ring; or optionally R⁸ and R⁷ join to form a ring;
R⁹ is selected from optionally substituted C1-C6 alkyl, or optionally substituted aryl;
m is 0, 1 or 2; and
n is 1-4.

One embodiment provides a compound having the structure of Formula (Ia), or a pharmaceutically acceptable salt or solvate thereof:

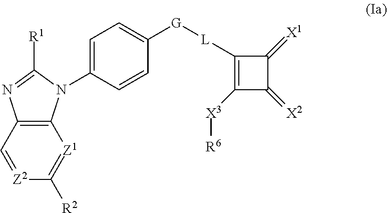

wherein:
G is O or CR⁴R⁵;
Z¹ s N, C—H, or C—R⁹;
Z² is N, C—H, or C—R³;
X¹ is O or S;
X² is O or S;
X³ is a bond, O, S, N—R⁷;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ and R⁵ are each independently hydrogen, halogen, —OH, or optionally substituted C1-C6 alkyl; or R⁴ and R⁵ together form an oxo; or R⁴ and R⁵ join together to form a carbocycle or heterocycle;
R⁶ is hydrogen, optionally substituted C1-C6 alkyl, optionally substituted C3-C7 cycloalkyl, and optionally substituted heterocyclyl; or R⁶ is absent and X³ and L join together to form a heterocycle;
R⁷ is selected from hydrogen, —OH, —NH₂, optionally substituted C1-C6 alkyl, optionally substituted C3-C7 cycloalkyl, and optionally substituted heterocyclyl; or optionally, R⁶ and R⁷ join together to form a heterocycle;
L is selected from —N(R⁸)—, or a divalent radical selected from:

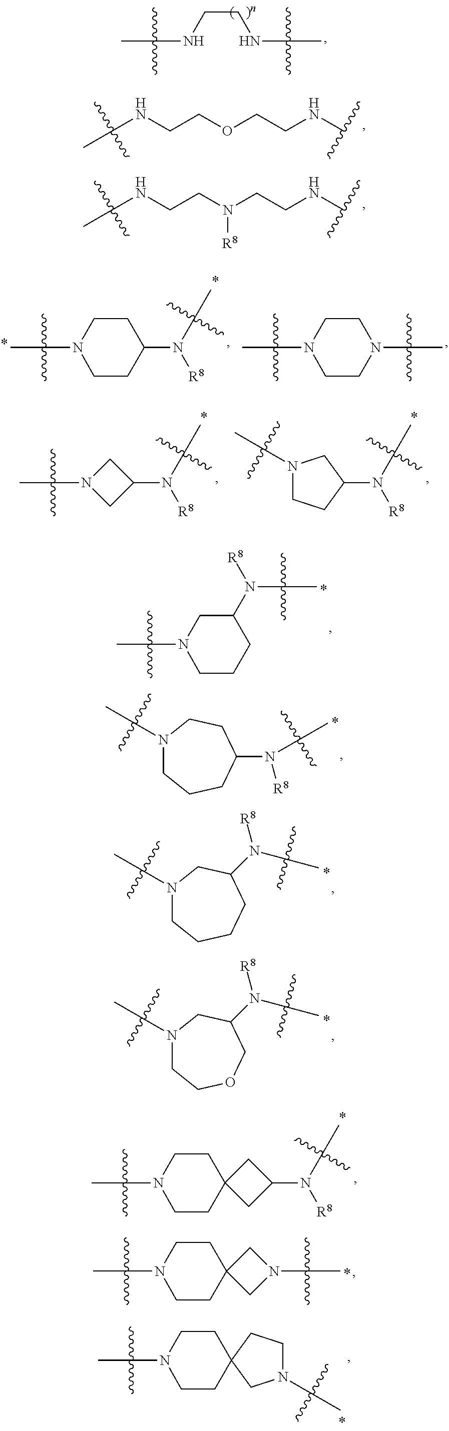

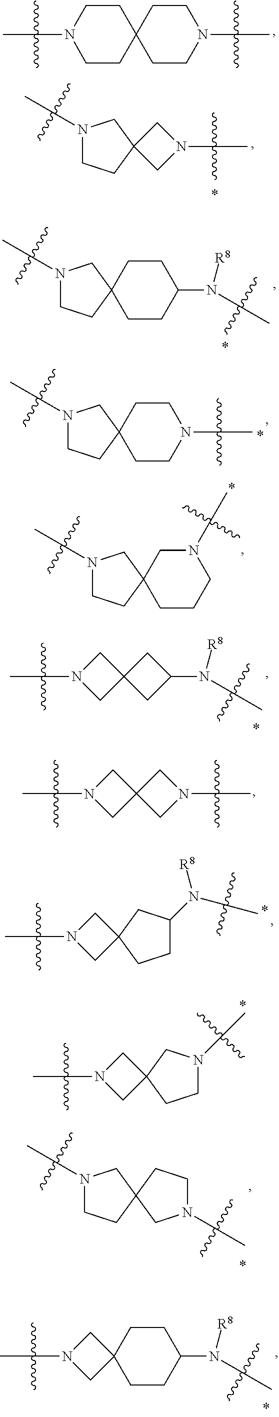

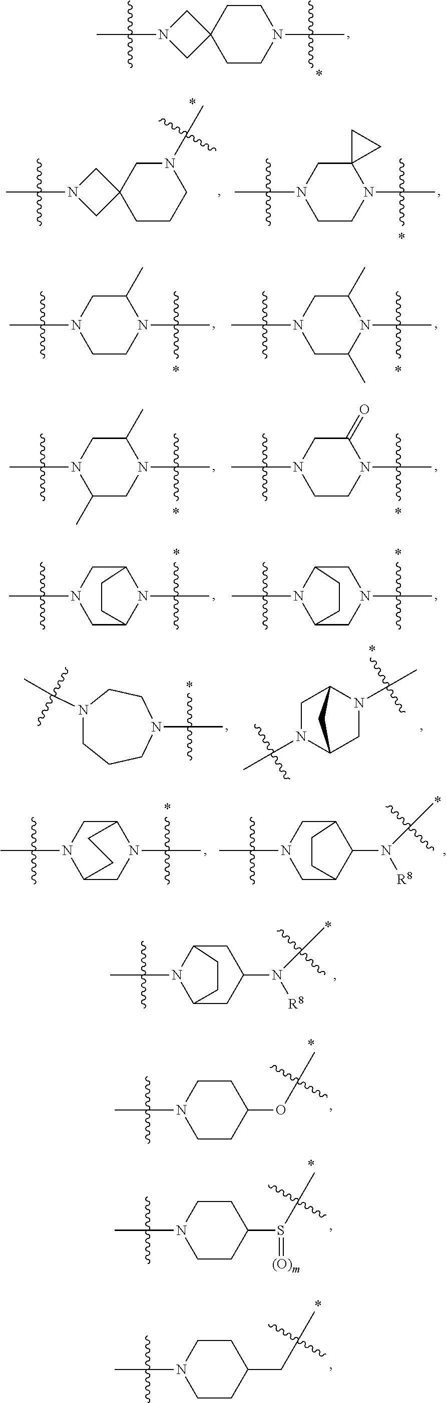

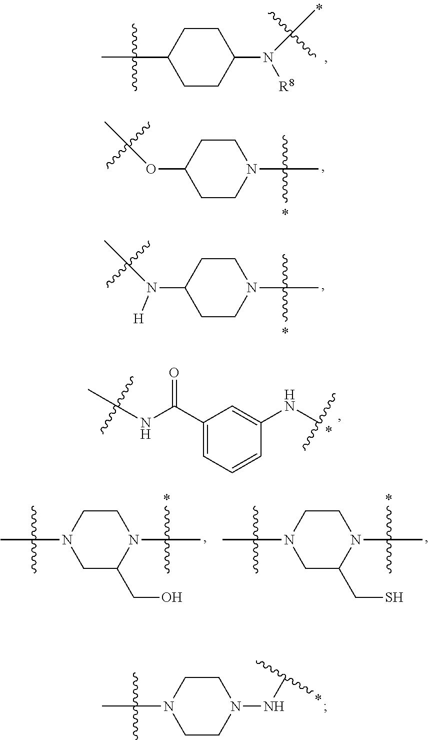

wherein the asterisk (*) indicates the bond to the squaric acid group;
R⁸ is hydrogen, optionally substituted C1-C6 alkyl, optionally substituted C3-C7 cycloalkyl, and optionally substituted heterocyclyl; or optionally R⁸ and R⁶ join to form a ring; or optionally R⁸ and R⁷ join to form a ring;
R⁹ is selected from optionally substituted C1-C6 alkyl, or optionally substituted aryl;
m is 0, 1 or 2; and
n is 1-4.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an optionally substituted alkyl is a haloalkyl. In other embodiments, an optionally substituted alkyl is a fluoroalkyl. In other embodiments, an optionally substituted alkyl is a —CF₃ group.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)N(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from optionally substituted alkyl, optionally substituted alkenyl, optionally substituted alkynyl, halo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{b}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond.

Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from optionally substituted alkyl, optionally substituted alkenyl, optionally substituted alkynyl, halo, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic, or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from optionally substituted alkyl, optionally substituted alkenyl, optionally substituted alkynyl, halo, fluoroalkyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from optionally substituted alkyl, optionally substituted cycloalkylalkyl, optionally substituted heterocyclylalkyl, optionally substituted alkenyl, optionally substituted alkynyl, halo, optionally substituted fluoroalkyl, optionally substituted haloalkenyl, optionally substituted haloalkynyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R is independently a direct bond or a straight or branched alkylene or alkenylene chain, and R^{c} is a straight or branched alkylene or alkenylene chain, and where each of the R^{a}, R, or R^{c} substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

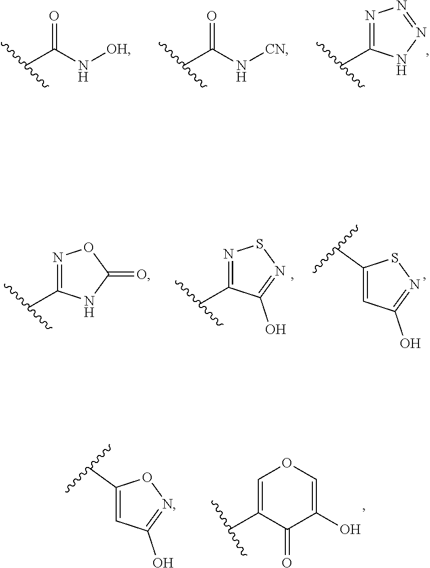

and the like.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

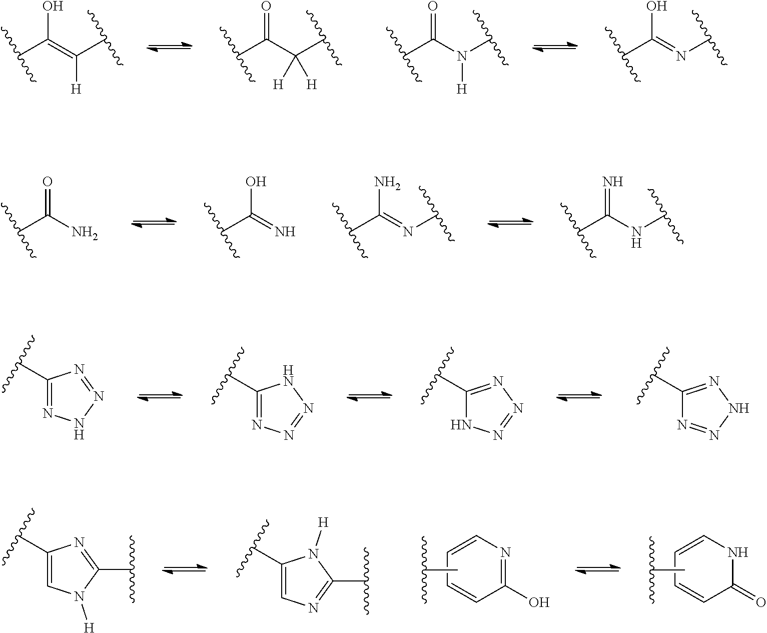

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O ¹⁴F, ¹⁵F, ¹⁶F, ¹⁷F, ¹⁸F, ³S, ³S, ³⁵S, ³⁶S, ³⁵Cl, ³⁷C, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

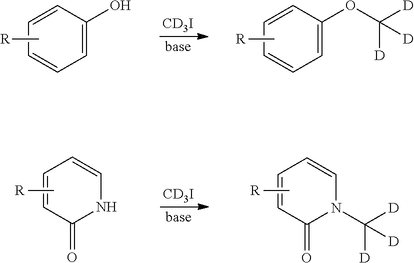

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

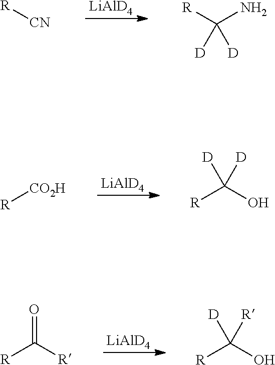

Deuterium gas and palladium catalyst are employed to reduce unsaturated carbon-carbon linkages and to perform a reductive substitution of aryl carbon-halogen bonds as illustrated, by way of example only, in the reaction schemes below.

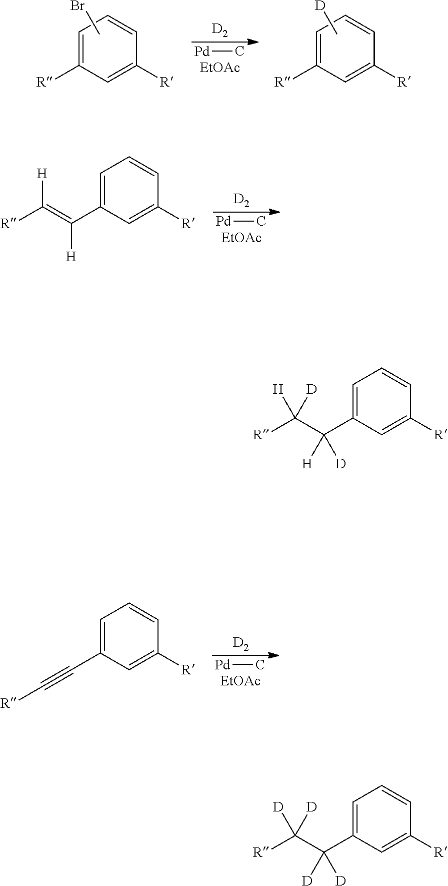

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the AKT1 inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### AKT1 Protein and Function

AKT, also known as protein kinase B (PKB), is a serine/threonine protein kinase with three isoforms, AKT1, AKT2, and AKT3. While the isoforms are encoded by different genes, they are highly homologous at the protein level and share a conserved domain structure comprising an N-terminal pleckstrin homology (PH) domain, a kinase domain, and a C-terminal regulatory domain comprising a hydrophobic moiety, which includes the regulatory serine residue (Nitulescu, G. M. et al., Int J Oncol., 2018; 53(6): 2319-2331).

AKT proteins play a crucial role in major cellular functions including cell cycle progression, cell size, regulation of glucose metabolism, transcription, protein synthesis, genome stability, and neovascularization. AKT proteins can block apoptosis by inactivation of pro-apoptotic proteins, and mediate cellular growth factors, promoting cell survival. AKT is a major downstream effector of nuclear factor-kappaB (NfκB), which may link AKT signaling to the nucleus of a cell.

AKT1 is ubiquitously expressed, whereas AKT2 is primarily expressed in insulin-responsive tissues, and AKT3 is primarily expressed in brain and testes. A shared phosphorylation site of AKT in the catalytic domain corresponds to a threonine residue; specifically, Thr308 in AKT1, Thr309 in AKT2, and Thr305 in AKT3. A shared phosphorylation site in the C-terminus of the protein cis a serine residue; specifically, Ser473 in AKT1, Ser474 in AKT2, and Ser472 in AKT3.

AKT is a key downstream mediator of the phosphoinositide-3-kinase (PI3K) signaling pathway. PI3Ks are activated by different compounds. For example, PI3Kα, PI3Kβ, and PI3Kδ, are activated by extracellular ligands binding to a transmembrane glycoprotein with enzymatic activity, receptor tyrosine kinases (RTKs). In contrast, PI3Kγ is activated by G-protein-compound receptors (GPCRs) and by RAS family of GTPases.

The AKT cascade can be activated by RTKs and G-protein-compound receptors (GPCRs), along with other signals including integrins, B cell receptors, T cell receptors, and cytokine receptors.

### AKT1 Mechanism

AKT is activated by a second phosphorylation at the regulatory serine residue, Ser473. Known phosphorylating agents of AKT at Ser473 include, but are not limited to PDK-1, integrin-linked kinase (ILK), members of the PI3K-related kinase (PIKK) family, and mammalian target of rapamycin (mTOR) (Nitulescu, G. M. et al., Int J Oncol., 2018; 53(6): 2319-2331).

mTOR is a key component in the AKT signaling pathway, which is a downstream member of AKT and important regulator for cell metabolism and growth. mTOR is also an activator which can directly phosphorylate AKT's regulatory serine residue, Ser473. mTOR forms a complex with rapamycin-insensitive companion of mTOR (RICTOR) (and other proteins) to form mTOR complex 2 (mTORC2), which can directly phosphorylate AKT Ser473. AKT can affect cell survival and growth because it can influence the tuberous sclerosis complex (TSC) ½ along the mTORC signaling pathway and inhibit pro-apoptotic proteins or signals.

AKT is known as a survival kinase and mediates cell survival and proliferation by inhibiting pathways including, but not limited to Bcl2 and MDM2, which promotes apoptosis. Studies have shown that the AKT signaling cascade have frequent malfunctions in various cancers, and may be associated with tumor aggressiveness (Nitulescu, G. M. et al., Int J Oncol., 2018; 53(6): 2319-2331). Malfunctions of AKT typically lead to enhanced proliferation, growth, survival, and resistance to apoptosis (Alwhaibi, A. et al., Pharmacol Res., 2019, 145: 104270). Malfunction and mis-regulation of AKT may lead to cancers such as but not limited to breast cancer, gastric carcinoma, glioblastoma, gliosarcomas, head and neck squamous cell carcinoma, ovarian cancer, pancreatic cancer, and prostate cancer.

Additionally, AKT1 has been found to be involved in invasion and migration of cancerous cells (Alwhaibi, A. et al., Pharmacol Res., 2019, 145: 104270). Researchers found that silencing the AKT1 isoform can abrogate specific types of cancer cell migration. However, there have been other studies which have demonstrated that activated AKT1 resulted in less metastatic propensity for lung metastatic lesion cells and breast cancer cells. AKT1 has also been identified as a key protein involved in angiogenesis, lung cancer, and tumorigenesis.

Furthermore, overexpression of AKT has been correlated to resistance to chemotherapeutic agents such as cisplatin, methotrexate, and paclitaxel. Thus, there remains a need to find AKT inhibitors given its role in cell survival and cancer proliferation.

Recently, it has been found that the AKT1 gene mutation E17K can affect cell growth, proliferation, survival, and migration of breast cancer cells, colorectal cancer cells, and ovarian cancer cells (Chen, Y. et al., Front Cell Dev Biol., 2020; 8: 573599). These mutations in the PH structural domain increase the binding of AKT1 to Phosphatidylinositol-3,4,5-triphosphate (PIP3) lipid ligand, which accelerates transfer of AKT from the cytoplasm to the cell membrane through formation of hydrogen bonds. Transfer of AKT into the cell membrane allows it to be further phosphorylated. Once fully activated, AKT can return to the cytoplasm, or go to the nucleus or other intracellular sites, and phosphorylate other substrate proteins to regulate cell function.

The E17K mutation enhances migration of breast cancer cells, and also enhances resistance to chemotherapeutic drugs. However, the E17K mutation can also selectively destroy chemo-resistant tumor-promoting AKT1 quiescent cancer cells, suggesting that the AKT1(E17K) mutation is crucial in the oncogenic/anti-tumor mechanism.

A major pathway that activates PI3K-AKT signaling pathway is somatic cell mutations, with the E17K mutation being the highest frequency of AKT1 mutations. It is nearly exclusively present in AKT1. The AKT1(E17K) is a recurrent somatic cell mutation predominantly in breast cancer, ovarian cancer, meningioma, and Proteus syndrome.

AKT1(E17K) mutations mediate the PI3K-AKT signaling cascade by expanding PIP lipid specificity, which causes conformational changes. This also enhances subcellular localization to accelerate localization of the PH structural domain to the plasma membrane. The E17K mutation increases PIP3 binding specificity by 7-fold and phosphatidylinositol-(4,5)-bisphosphate (PIP2) by 100-fold.

The AKT1(E17K) mutation also causes rapid conformational changes in the AKT1 PH structural domain. The conformational changes to this domain result in a 4.5-fold increase in its membrane localization, which can result in excessive phosphorylation. The AKT1(E17K) mutation can also result in enhanced subcellular localization by increasing the transient expression.

Given the conformational and signaling effects of the AKT1(E17K) mutation, this target may be useful for targeted treatment of cancers.

### Prior Art AKT1 Inhibitors

Most AKT inhibitors targeting the ATP binding site are non-selective against the three isoforms, as well as having poor to no selectivity against other structurally similar kinases. Thus, there remains a need to develop new and novel AKT inhibitors. These ATP targeting inhibitors are classified as aminofurazans, azepane derivatives, isoquinoline-5-sulfonamides, phenylpyrazole derivatives, thiophene carboxamide derivatives, and thiazole carboxamide derivatives.

There are also ATP non-competitive AKT inhibitors which are allosteric modulators which has greater specificity than the ATP targeting inhibitors. Many of these allosteric modulator inhibitors are classified as purine derivatives, thiourea derivatives, alkylphospholipids, sulfonamides, 2,3-diphenylquinoxaline analogs, and indole-3-carbinol derivatives.

### Novel AKT1 Inhibitory Compounds

In one aspect, provided herein is a AKT1 inhibitory compound.

One embodiment provides a compound having the structure of Formula (I), or a pharmaceutically acceptable salt or solvate thereof:

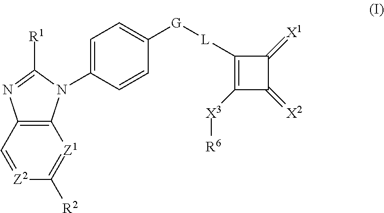

wherein:
G is O or CR⁴R⁵;
Z¹ is N, C—H, or C—R⁹;
Z² is N, C—H, or C—R³;
X¹ is O or S;
X² is O or S;
X³ is a bond, O, S, N—R⁷;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ and R⁵ are each independently hydrogen, halogen, —OH, or optionally substituted C1-C6 alkyl; or R⁴ and R⁵ together form an oxo; or R⁴ and R⁵ join together to form a carbocycle or heterocycle;
R⁶ is hydrogen, optionally substituted C1-C6 alkyl, optionally substituted C3-C7 cycloalkyl, and optionally substituted heterocyclyl; or R⁶ is absent and X³ and L join together to form a heterocycle;
R⁷ is selected from hydrogen, —OH, —NH₂, optionally substituted C1-C6 alkyl, optionally substituted C3-C7 cycloalkyl, and optionally substituted heterocyclyl; or optionally, R⁶ and R⁷ join together to form a heterocycle;
L is selected from —N(R⁸)—, or a divalent radical selected from:

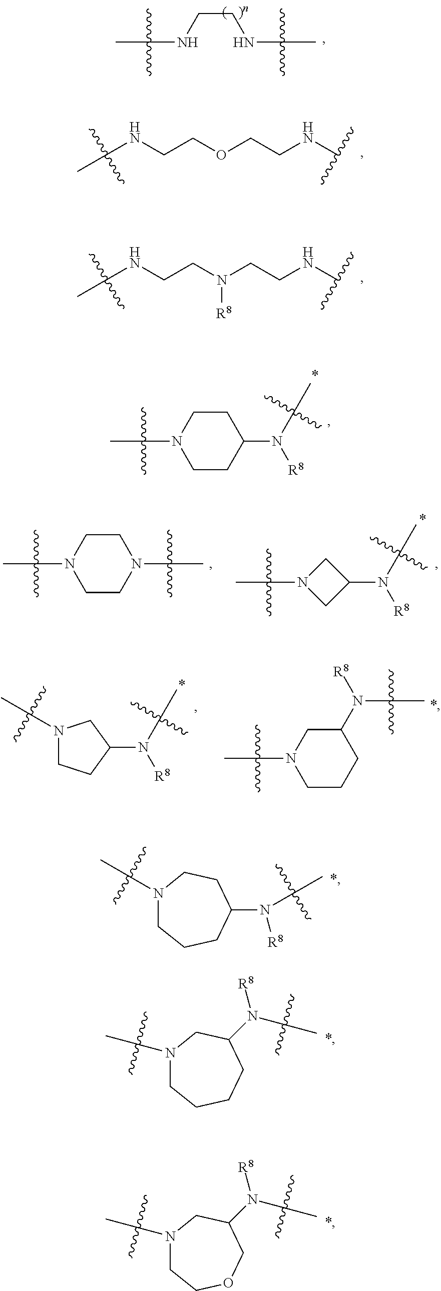

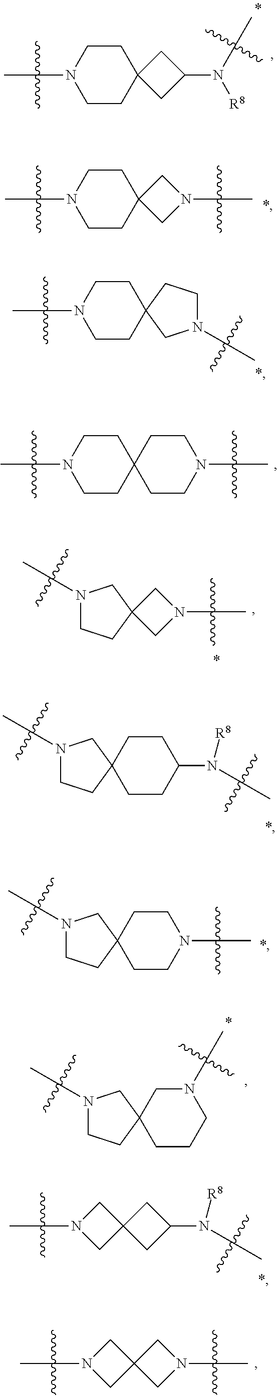

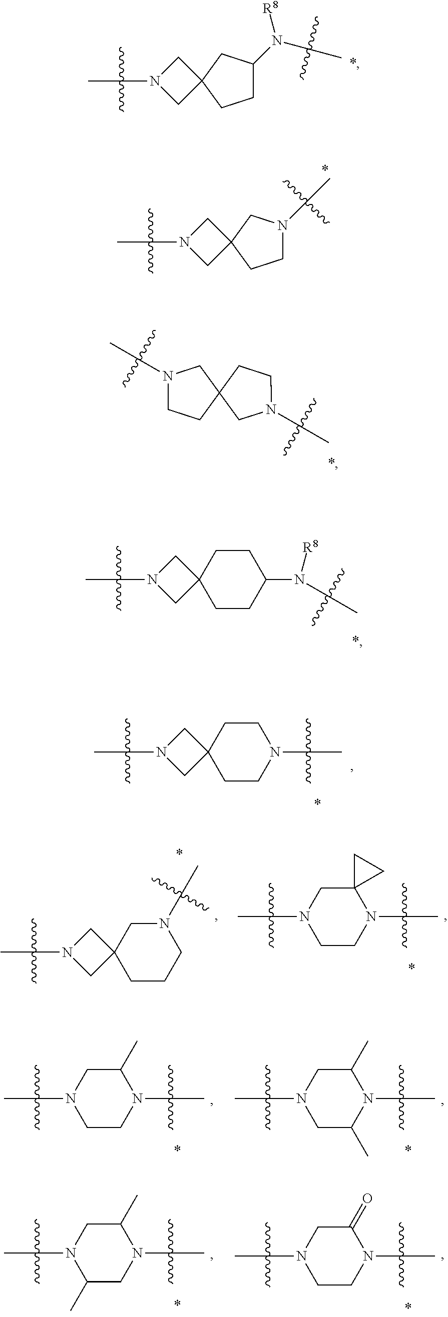

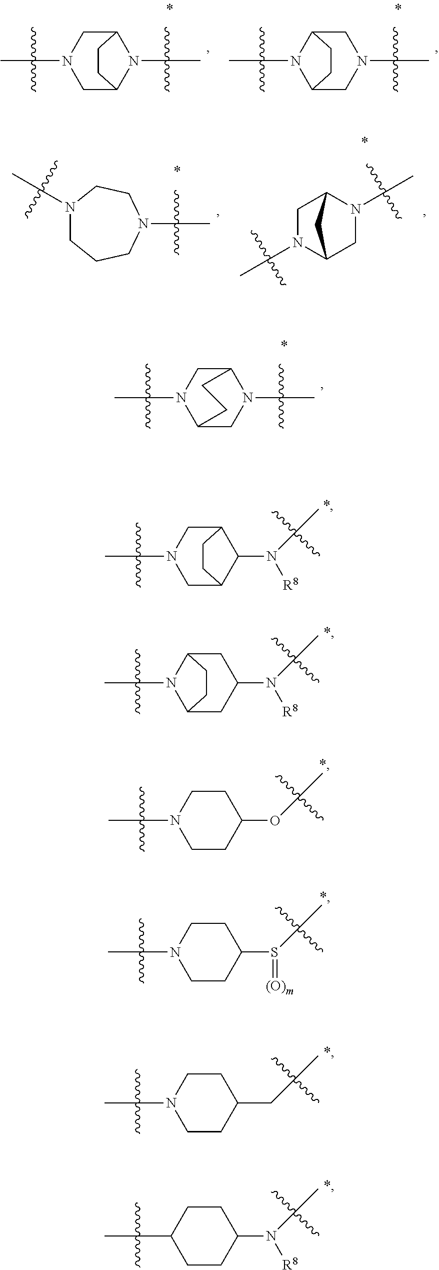

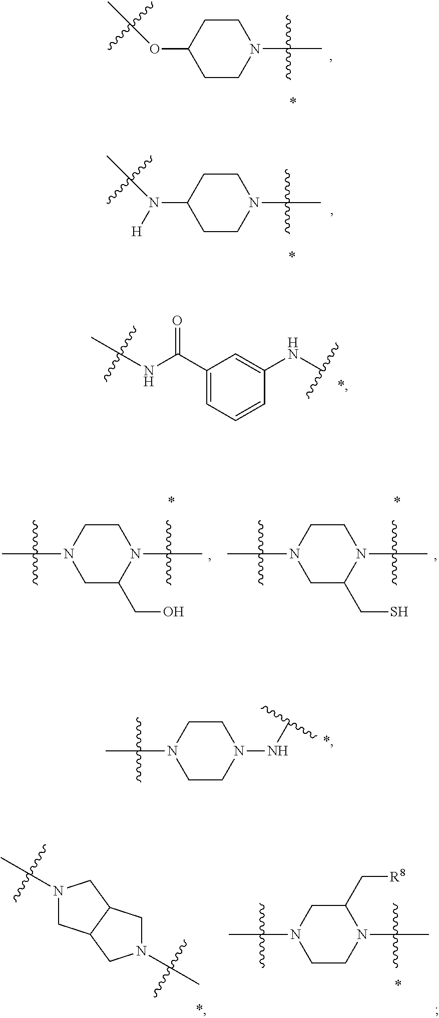

wherein the asterisk (*) indicates the bond to the squaric acid group;
R⁸ is hydrogen, optionally substituted C1-C6 alkyl, optionally substituted C3-C7 cycloalkyl, and optionally substituted heterocyclyl; or optionally R⁸ and R⁶ join to form a ring; or optionally R⁸ and R⁷ join to form a ring;
R⁹ is selected from optionally substituted C1-C6 alkyl, or optionally substituted aryl;
m is 0, 1 or 2; and
n is 1-4.

One embodiment provides a compound having the structure of Formula (Ia), or a pharmaceutically acceptable salt or solvate thereof:

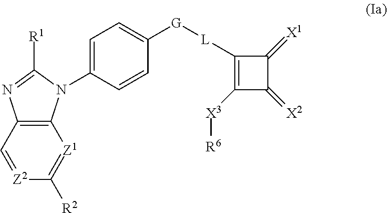

wherein:
G is O or CR⁴R⁵;
Z¹ is N, C—H, or C—R⁹;
Z² is N, C—H, or C—R³;
X¹ is O or S;
X² is O or S;
X³ is a bond, O, S, N—R⁷;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ and R⁵ are each independently hydrogen, halogen, —OH, or optionally substituted C1-C6 alkyl; or R⁴ and R⁵ together form an oxo; or R⁴ and R⁵ join together to form a carbocycle or heterocycle;
R⁶ is hydrogen, optionally substituted C1-C6 alkyl, optionally substituted C3-C7 cycloalkyl, and optionally substituted heterocyclyl; or R⁶ is absent and X³ and L join together to form a heterocycle;
R⁷ is selected from hydrogen, —OH, —NH₂, optionally substituted C1-C6 alkyl, optionally substituted C3-C7 cycloalkyl, and optionally substituted heterocyclyl; or optionally, R⁶ and R⁷ join together to form a heterocycle;
L is selected from —N(R⁸)—, or a divalent radical selected from:

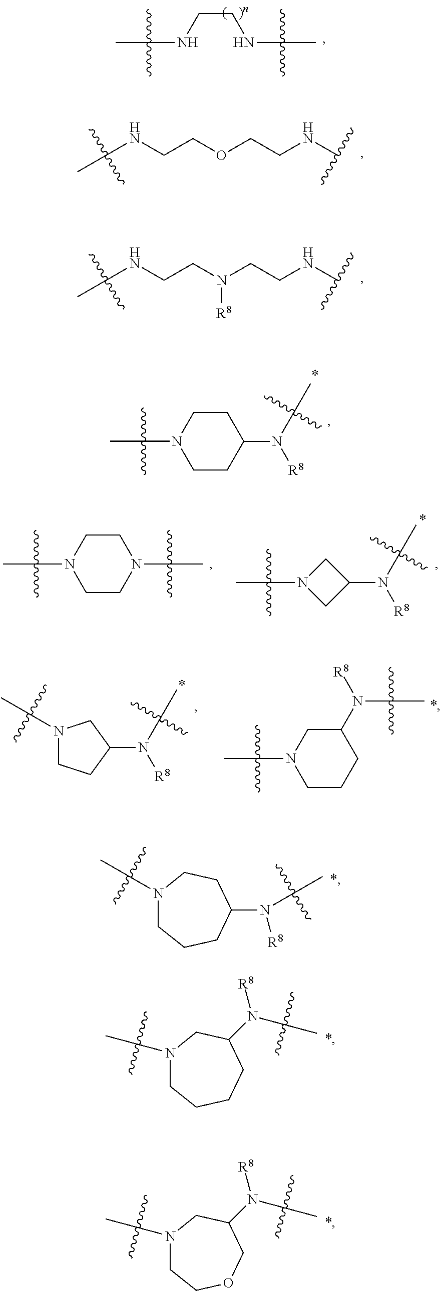

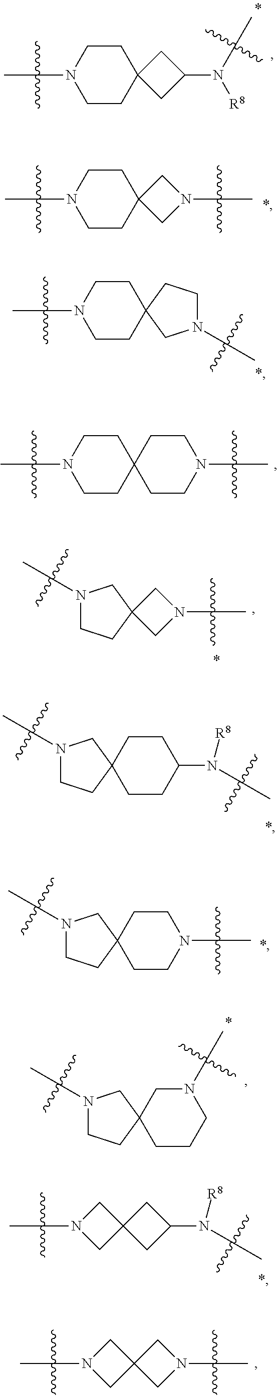

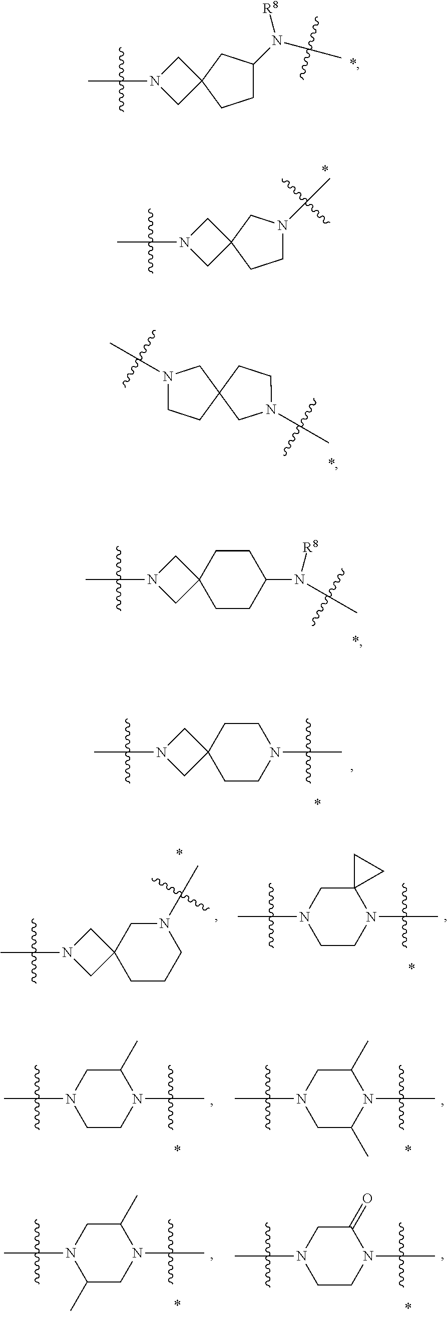

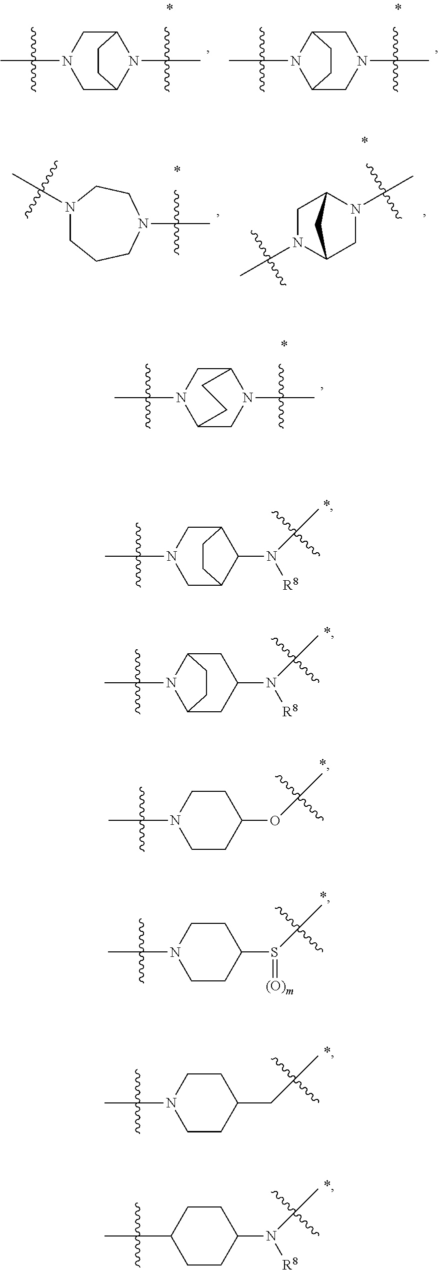

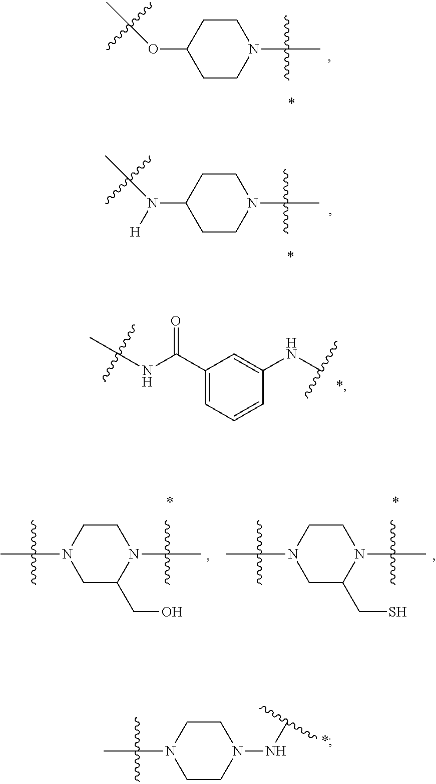 [SMILES: *OC1CCN(*)CC1 ; *NC1CCN(*)CC1 ; *NC(=O)c1cccc(N*)c1 ; *N1CCN(*)C(CO)C1 ; *N1CCN(*)C(CS)C1 ; *NN1CCN(*)CC1]

wherein the asterisk (*) indicates the bond to the squaric acid group;
R⁸ is hydrogen, optionally substituted C1-C6 alkyl, optionally substituted C3-C7 cycloalkyl, and optionally substituted heterocyclyl; or optionally R⁸ and R⁶ join to form a ring; or optionally R⁸ and R⁷ join to form a ring;
R⁹ is selected from optionally substituted C1-C6 alkyl, or optionally substituted aryl;
m is 0, 1 or 2; and
n is 1-4.

Another embodiment provides the compound of Formula (I), or pharmaceutically acceptable salt or solvate thereof, wherein G is O. Another embodiment provides the compound of Formula (I), or pharmaceutically acceptable salt or solvate thereof, wherein G is CR⁴R⁵.

Another embodiment provides the compound of Formula (I), or pharmaceutically acceptable salt or solvate thereof, wherein Z¹ is N.

Another embodiment provides the compound of Formula (I), or pharmaceutically acceptable salt or solvate thereof, wherein Z² is C—H. Another embodiment provides the compound of Formula (I), or pharmaceutically acceptable salt or solvate thereof, wherein Z² is C—R³.

Another embodiment provides the compound of Formula (I), or pharmaceutically acceptable salt or solvate thereof, wherein X¹ is O. Another embodiment provides the compound of Formula (I), or pharmaceutically acceptable salt or solvate thereof, wherein X¹ is S.

Another embodiment provides the compound of Formula (I), or pharmaceutically acceptable salt or solvate thereof, wherein X² is O. Another embodiment provides the compound of Formula (I), or pharmaceutically acceptable salt or solvate thereof, wherein X² is O.

Another embodiment provides the compound of Formula (I), or pharmaceutically acceptable salt or solvate thereof, wherein X³ is a bond. Another embodiment provides the compound of Formula (I), or pharmaceutically acceptable salt or solvate thereof, wherein X³ is O. Another embodiment provides the compound of Formula (I), or pharmaceutically acceptable salt or solvate thereof, wherein X³ is S. Another embodiment provides the compound of Formula (I), or pharmaceutically acceptable salt or solvate thereof, wherein X³ is N—R⁷.

Another embodiment provides the compound of Formula (I), or pharmaceutically acceptable salt or solvate thereof, wherein R¹ is optionally substituted heteroaryl. Another embodiment provides the compound of Formula (I), or pharmaceutically acceptable salt or solvate thereof, wherein the optionally substituted heteroaryl is an optionally substituted pyridyl.

Another embodiment provides the compound of Formula (I), or pharmaceutically acceptable salt or solvate thereof, wherein R² is optionally substituted aryl. Another embodiment provides the compound of Formula (I), or pharmaceutically acceptable salt or solvate thereof, wherein the optionally substituted aryl is an optionally substituted phenyl.

Another embodiment provides the compound of Formula (I), or pharmaceutically acceptable salt or solvate thereof, wherein R⁴ is hydrogen. Another embodiment provides the compound of Formula (I), or pharmaceutically acceptable salt or solvate thereof, wherein R⁵ is hydrogen. Another embodiment provides the compound of Formula (I), or pharmaceutically acceptable salt or solvate thereof, wherein R⁴ and R⁵ together form an oxo.

Another embodiment provides the compound of Formula (I), or pharmaceutically acceptable salt or solvate thereof, wherein R⁶ is hydrogen. Another embodiment provides the compound of Formula (I), or pharmaceutically acceptable salt or solvate thereof, wherein R⁶ is optionally substituted C1-C6 alkyl. Another embodiment provides the compound of Formula (I), or pharmaceutically acceptable salt or solvate thereof, wherein R⁶ is optionally substituted C3-C7 cycloalkyl. Another embodiment provides the compound of Formula (I), or pharmaceutically acceptable salt or solvate thereof, wherein R⁶ is optionally substituted heterocyclyl. Another embodiment provides the compound of Formula (I), or pharmaceutically acceptable salt or solvate thereof, wherein R⁶ is absent and X³ and L join together to form a heterocycle.

Another embodiment provides the compound of Formula (I), or pharmaceutically acceptable salt or solvate thereof, wherein R⁷ is selected from hydrogen, or optionally substituted C1-C6 alkyl.

Another embodiment provides the compound of Formula (I), or pharmaceutically acceptable salt or solvate thereof, wherein R⁶ and R⁷ join together to form a heterocycle.

Another embodiment provides the compound of Formula (I), or pharmaceutically acceptable salt or solvate thereof, wherein L is —N(R′)—.

Another embodiment provides the compound of Formula (I), or pharmaceutically acceptable salt or solvate thereof, wherein L is selected from:

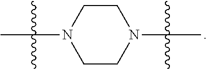 [SMILES: *N1CCN(*)CC1]

Another embodiment provides the compound of Formula (I), or pharmaceutically acceptable salt or solvate thereof, wherein L is selected from:

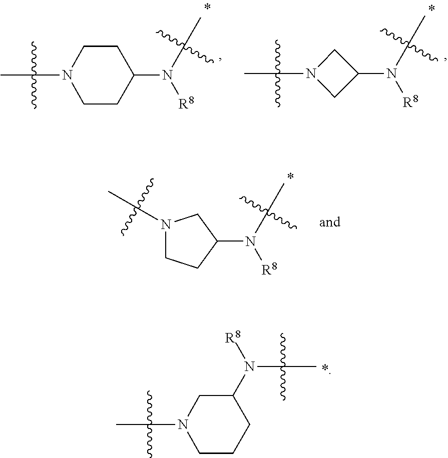

Another embodiment provides the compound of Formula (I), or pharmaceutically acceptable salt or solvate thereof, wherein L is selected from:

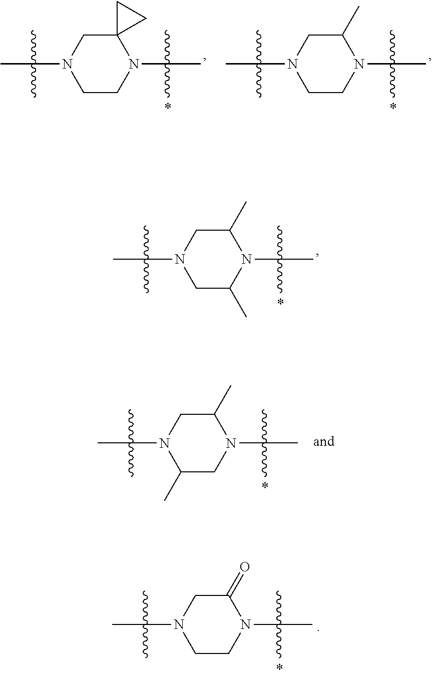

Another embodiment provides the compound of Formula (I), or pharmaceutically acceptable salt or solvate thereof, wherein L is selected from:

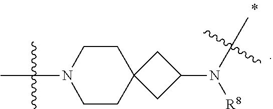

Another embodiment provides the compound of Formula (I), or pharmaceutically acceptable salt or solvate thereof, wherein L is selected from:

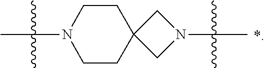 [SMILES: *N1CCC2(CC1)CN(*)C2]

Another embodiment provides the compound of Formula (I), or pharmaceutically acceptable salt or solvate thereof, wherein L is selected from:

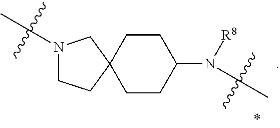

Another embodiment provides the compound of Formula (I), or pharmaceutically acceptable salt or solvate thereof, wherein L is selected from:

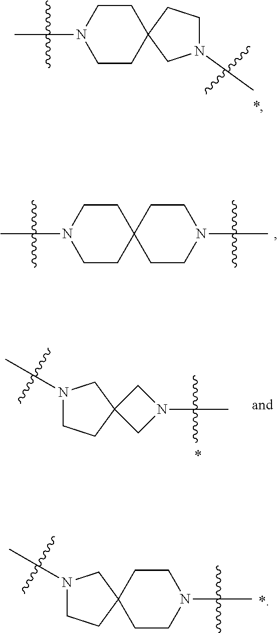 [SMILES: *N1CCC2(CC1)CCN(*)C2 ; *N1CCC2(CC1)CCN(*)CC2 ; *N1CCC2(C1)CN(*)C2 ; *N1CCC2(CC1)CCN(*)C2]

Another embodiment provides the compound of Formula (I), or pharmaceutically acceptable salt or solvate thereof, wherein L is selected from:

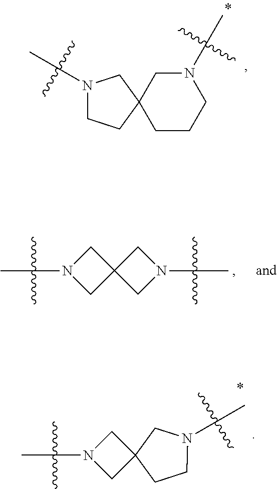 [SMILES: *N1CCCC2(CCN(*)C2)C1 ; *N1CC2(C1)CN(*)C2 ; *N1CCC2(C1)CN(*)C2]

Another embodiment provides the compound of Formula (I), or pharmaceutically acceptable salt or solvate thereof, wherein L is selected from:

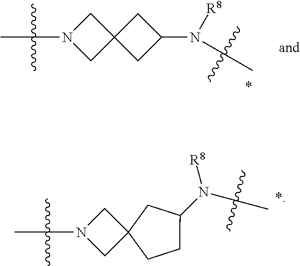

Another embodiment provides the compound of Formula (I), or pharmaceutically acceptable salt or solvate thereof, wherein L is selected from:

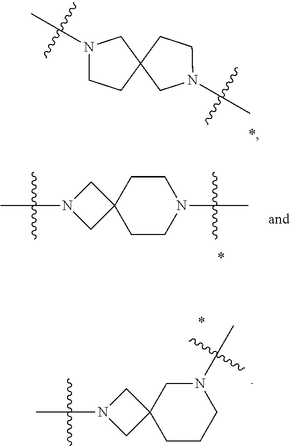 [SMILES: *N1CCC2(CCN(*)C2)C1 ; *N1CCC2(CC1)CN(*)C2 ; *N1CCCC2(C1)CN(*)C2]

Another embodiment provides the compound of Formula (I), or pharmaceutically acceptable salt or solvate thereof, wherein L is selected from:

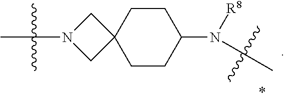

Another embodiment provides the compound of Formula (I), or pharmaceutically acceptable salt or solvate thereof, wherein R⁸ is hydrogen or optionally substituted C1-C6 alkyl.

One embodiment provides an AKT1 inhibitory compound, or a pharmaceutically acceptable salt or solvate thereof, having a structure presented in Table 1.

TABLE 1

| Synthetic | | |
|---|---|---|
| Chemistry | | |
| Example | Compound Structure | Compound Name |
| 1 | 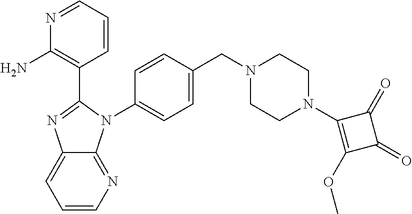 [SMILES: COc1c(N2CCN(Cc3ccc(-n4c(-c5cccnc5N)nc5cccnc54)cc3)CC2)c(=O)c1=O] | 3-(4-(4-(2-(2-aminopyridin-3-yl)- 3H-imidazo[4,5-b]pyridin-3- yl)benzyl)piperazin-1-yl)-4- methoxycyclobut-3-ene-1,2-dione |
| 2 | 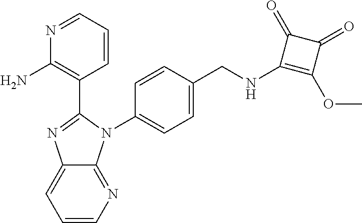 [SMILES: COc1c(NCc2ccc(-n3c(-c4cccnc4N)nc4cccnc43)cc2)c(=O)c1=O] | 3-((4-(2-(2-aminopyridin-3-yl)- 3H-imidazo[4,5-b]pyridin-3- yl)benzyl)amino)-4- methoxycyclobut-3-ene-1,2-dione |
| 3 | 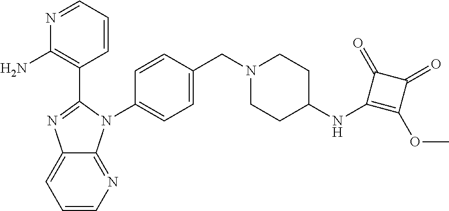 [SMILES: COc1c(NC2CCN(Cc3ccc(-n4c(-c5cccnc5N)nc5cccnc54)cc3)CC2)c(=O)c1=O] | 3-((1-(4-(2-(2-aminopyridin-3-yl)- 3H-imidazo[4,5-b ]pyridin-3- yl)benzyl)piperidin-4-yl)amino)-4- methoxycyclobut-3-ene-1,2-dione |
| 4 | 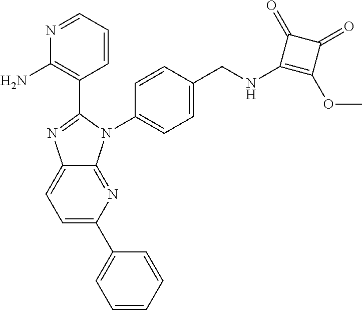 [SMILES: COc1c(NCc2ccc(-n3c(-c4cccnc4N)nc4ccc(-c5ccccc5)nc43)cc2)c(=O)c1=O] | 3-((4-(2-(2-aminopyridin-3-yl)-5- phenyl-3H-imidazo[4,5-b]pyridin- 3-yl)benzyl)amino)-4- methoxycyclobut-3-ene-1,2-dione |
| 5 | 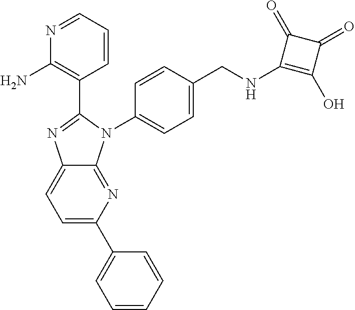 [SMILES: Nc1ncccc1-c1nc2ccc(-c3ccccc3)nc2n1-c1ccc(CNc2c(O)c(=O)c2=O)cc1] | 3-((4-(2-(2-aminopyridin-3-yl)-5- phenyl-3H-imidazo[4,5-b]pyridin- 3-yl)benzyl)amino)-4- hydroxycyclobut-3-ene-1,2-dione |
| 6 | 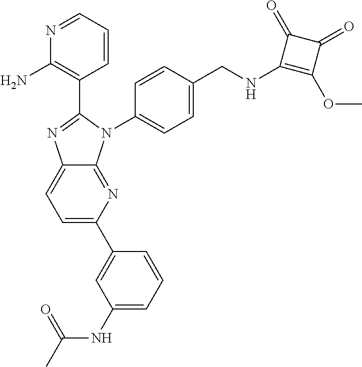 [SMILES: COc1c(NCc2ccc(-n3c(-c4cccnc4N)nc4ccc(-c5cccc(NC(C)=O)c5)nc43)cc2)c(=O)c1=O] | N-(3-(2-(2-aminopyridin-3-yl)-3- (4-(((2-methoxy-3,4- dioxocyclobut-1-en-1- yl)amino)methyl)phenyl)-3H- imidazo[4,5-b]pyridin-5- yl)phenyl)acetamide |
| 7 | 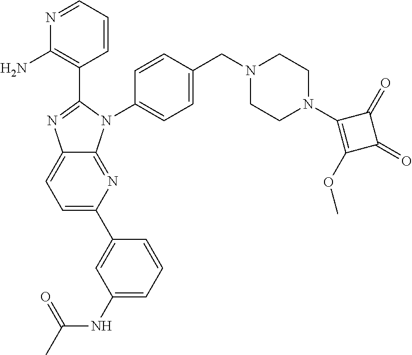 [SMILES: COc1c(N2CCN(Cc3ccc(-n4c(-c5cccnc5N)nc5ccc(-c6cccc(NC(C)=O)c6)nc54)cc3)CC2)c(=O)c1=O] | N-(3-(2-(2-aminopyridin-3-yl)-3- (4-((4-(2-methoxy-3,4- dioxocyclobut-1-en-1- yl)piperazin-1-yl)methyl)phenyl)- 3H-imidazo[4,5-b]pyridin-5- yl)phenyl)acetamide |
| 8 | 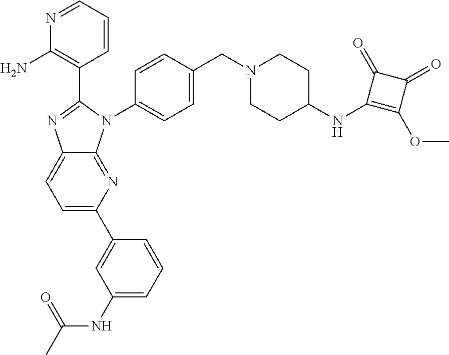 [SMILES: COc1c(NC2CCN(Cc3ccc(-n4c(-c5cccnc5N)nc5ccc(-c6cccc(NC(C)=O)c6)nc54)cc3)CC2)c(=O)c1=O] | N-(3-(2-(2-aminopyridin-3-yl)-3- (4-((4-((2-methoxy-3,4- dioxocyclobut-1-en-1- yl)amino)piperidin-1- yl)methyl)phenyl)-3H- imidazo[4,5-b]pyridin-5- yl)phenyl)acetamide |
| 9 | 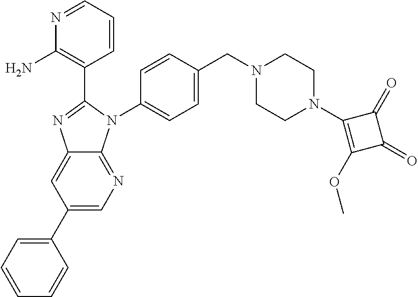 [SMILES: COc1c(N2CCN(Cc3ccc(-n4c(-c5cccnc5N)nc5cc(-c6ccccc6)cnc54)cc3)CC2)c(=O)c1=O] | 3-(4-(4-(2-(2-aminopyridin-3-yl)- 6-phenyl-3H-imidazo[4,5- b]pyridin-3-yl)benzyl)piperazin-1- yl)-4-methoxycyclobut-3-ene-1,2- dione |
| 10 | 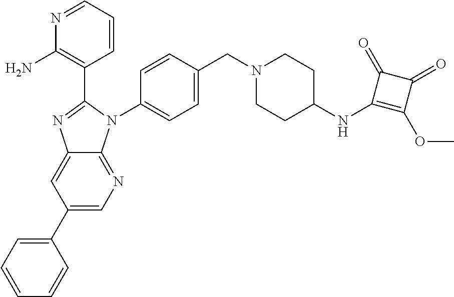 [SMILES: COc1c(NC2CCN(Cc3ccc(-n4c(-c5cccnc5N)nc5cc(-c6ccccc6)cnc54)cc3)CC2)c(=O)c1=O] | 3-((1-(4-(2-(2-aminopyridin-3-yl)- 6-phenyl-3H-imidazo[4,5- b]pyridin-3-yl)benzyl)piperidin-4- yl)amino)-4-methoxycyclobut-3- ene-1,2-dione |
| 11 | 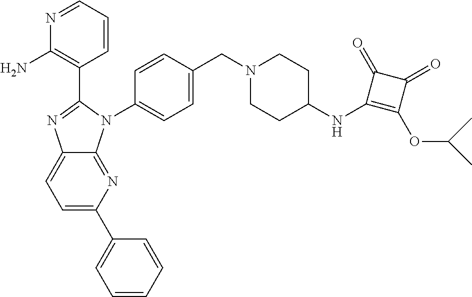 [SMILES: CC(C)Oc1c(NC2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)amino)-4-isopropoxycyclobut- 3-ene-1,2-dione |
| 12 | 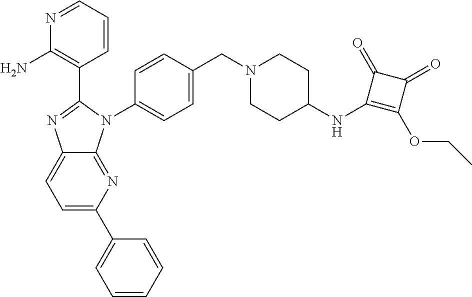 [SMILES: CCOc1c(NC2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)amino)-4-ethoxycyclobut-3- ene-1,2-dione |
| 13 | 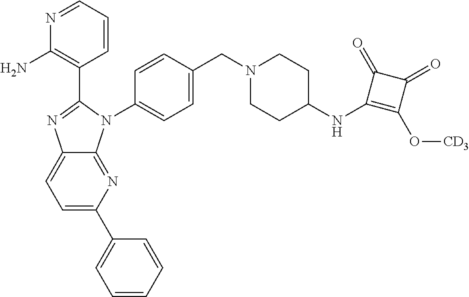 [SMILES: [2H]C([2H])([2H])Oc1c(NC2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)amino)-4-(methoxy- d3)cyclobut-3-ene-1,2-dione |
| 14 | 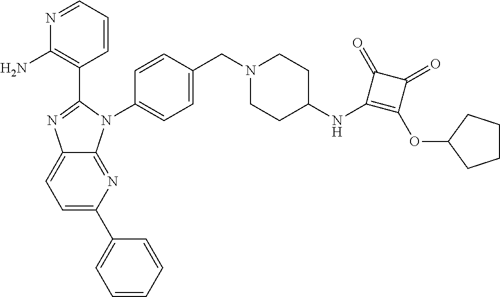 [SMILES: Nc1ncccc1-c1nc2ccc(-c3ccccc3)nc2n1-c1ccc(CN2CCC(Nc3c(OC4CCCC4)c(=O)c3=O)CC2)cc1] | 3-((1-(4-(2-(2-aminopyridin-3-yl)- 5-phenyl-3H-imidazo[4,5- b]pyridin-3-yl)benzyl)piperidin-4- yl)amino)-4- (cyclopentyloxy)cyclobut-3-ene- 1,2-dione |
| 15 | 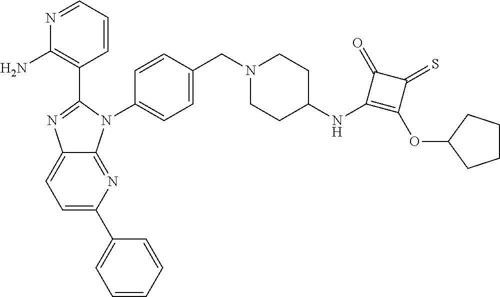 [SMILES: Nc1ncccc1-c1nc2ccc(-c3ccccc3)nc2n1-c1ccc(CN2CCC(Nc3c(OC4CCCC4)c(=S)c3=O)CC2)cc1] | 2-((1-(4-(2-(2-aminopyridin-3-yl)- 5-phenyl-3H-imidazo[4,5- b]pyridin-3-yl)benzyl)piperidin-4- yl)amino)-3-(cyclopentyloxy)-4- thioxocyclobut-2-en-1-one |
| 16 | 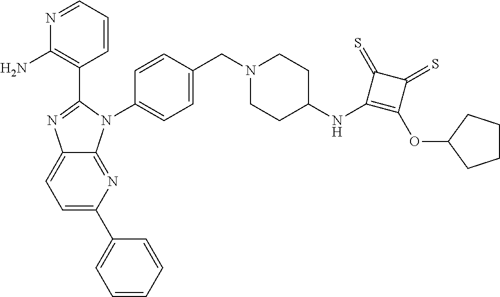 [SMILES: Nc1ncccc1-c1nc2ccc(-c3ccccc3)nc2n1-c1ccc(CN2CCC(Nc3c(OC4CCCC4)c(=S)c3=S)CC2)cc1] | 3-((1-(4-(2-(2-aminopyridin-3-yl)- 5-phenyl-3H-imidazo[4,5- b]pyridin-3-yl)benzyl)piperidin-4- yl)amino)-4- (cyclopentyloxy)cyclobut-3-ene- 1,2-dithione |
| 17 | 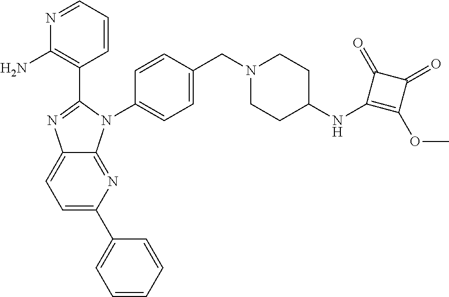 [SMILES: COc1c(NC2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)amino)-4-methoxycyclobut-3- ene-1,2-dione |
| 18 | 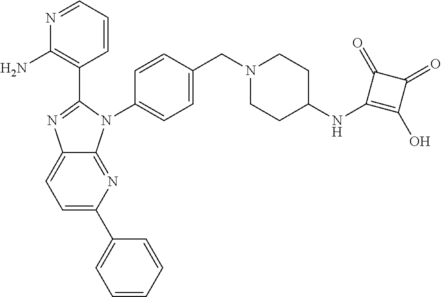 [SMILES: Nc1ncccc1-c1nc2ccc(-c3ccccc3)nc2n1-c1ccc(CN2CCC(Nc3c(O)c(=O)c3=O)CC2)cc1] | 3-((1-(4-(2-(2-aminopyridin-3-yl)- 5-phenyl-3H-imidazo[4,5- b]pyridin-3-yl)benzyl)piperidin-4- yl)amino)-4-hydroxycyclobut-3- ene-1,2-dione |
| 19 | 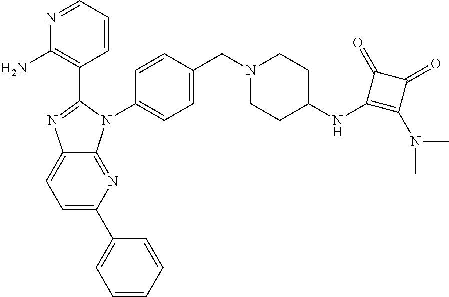 [SMILES: CN(C)c1c(NC2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)amino)-4- (dimethylamino)cyclobut-3-ene- 1,2-dione |
| 20 | 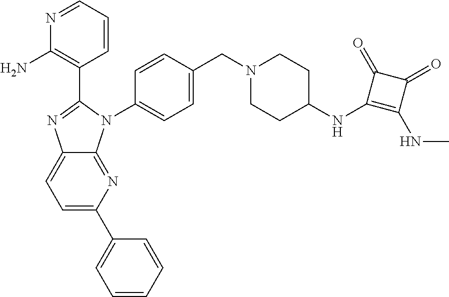 [SMILES: CNc1c(NC2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)amino)-4- (methylamino)cyclobut-3-ene-1,2- dione |
| 21 | 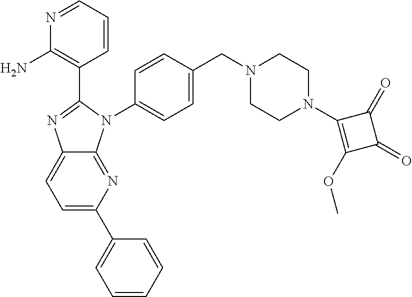 [SMILES: COc1c(N2CCN(Cc3ccc(-n4c(-c5cccnc5N)nc5ccc(-c6ccccc6)nc54)cc3)CC2)c(=O)c1=O] | 3-(4-(4-(2-(2-aminopyridin-3-yl)- 5-phenyl-3H-imidazo[4,5- b]pyridin-3-yl)benzyl)piperazin-1- yl)-4-methoxycyclobut-3-ene-1,2- dione |
| 22 | 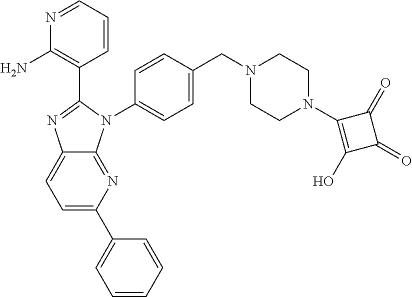 [SMILES: Nc1ncccc1-c1nc2ccc(-c3ccccc3)nc2n1-c1ccc(CN2CCN(c3c(O)c(=O)c3=O)CC2)cc1] | 3-(4-(4-(2-(2-aminopyridin-3-yl)- 5-phenyl-3H-imidazo[4,5- b]pyridin-3-yl)benzyl)piperazin-1- yl)-4-hydroxycyclobut-3-ene-1,2- dione |
| 23 | 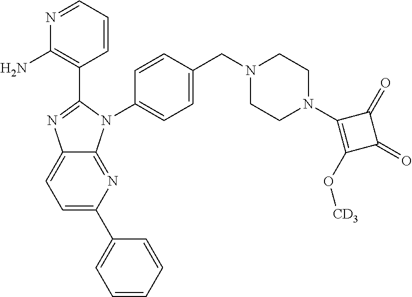 [SMILES: [2H]C([2H])([2H])Oc1c(N2CCN(Cc3ccc(-n4c(-c5cccnc5N)nc5ccc(-c6ccccc6)nc54)cc3)CC2)c(=O)c1=O] | 3-(4-(4-(2-(2-aminopyridin-3-yl)- 5-phenyl-3H-imidazo[4,5- b]pyridin-3-yl)benzyl)piperazin-1- yl)-4-(methoxy-d3)cyclobut-3- ene-1,2-dione |
| 24 | 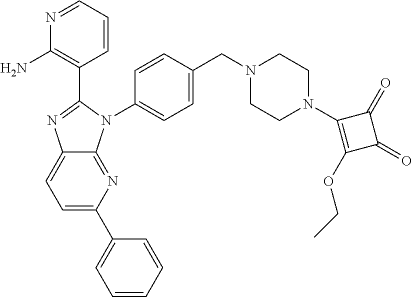 [SMILES: CCOc1c(N2CCN(Cc3ccc(-n4c(-c5cccnc5N)nc5ccc(-c6ccccc6)nc54)cc3)CC2)c(=O)c1=O] | 3-(4-(4-(2-(2-aminopyridin-3-yl)- 5-phenyl-3H-imidazo[4,5- b]pyridin-3-yl)benzyl)piperazin-1- yl)-4-ethoxycyclobut-3-ene-1,2- dione |
| 25 | 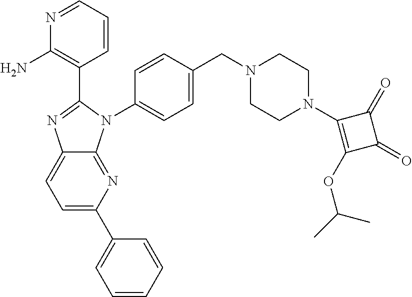 [SMILES: CC(C)Oc1c(N2CCN(Cc3ccc(-n4c(-c5cccnc5N)nc5ccc(-c6ccccc6)nc54)cc3)CC2)c(=O)c1=O] | 3-(4-(4-(2-(2-aminopyridin-3-yl)- 5-phenyl-3H-imidazo[4,5- b]pyridin-3-yl)benzyl)piperazin-1- yl)-4-isopropoxycyclobut-3-ene- 1,2-dione |
| 26 | 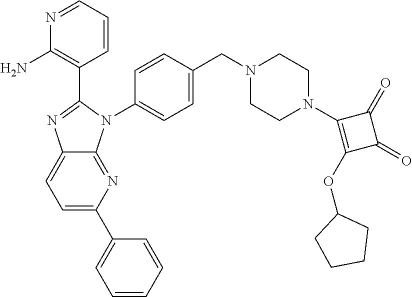 [SMILES: Nc1ncccc1-c1nc2ccc(-c3ccccc3)nc2n1-c1ccc(CN2CCN(c3c(OC4CCCC4)c(=O)c3=O)CC2)cc1] | 3-(4-(4-(2-(2-aminopyridin-3-yl)- 5-phenyl-3H-imidazo[4,5- b]pyridin-3-yl)benzyl)piperazin-1- yl)-4-(cyclopentyloxy)cyclobut-3- ene-1,2-dione |
| 27 | 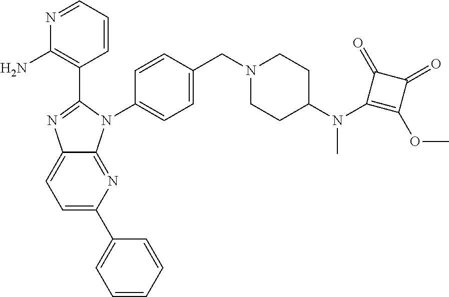 [SMILES: COc1c(N(C)C2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)(methyl)amino)-4- methoxycyclobut-3-ene-1,2-dione |
| 28 | 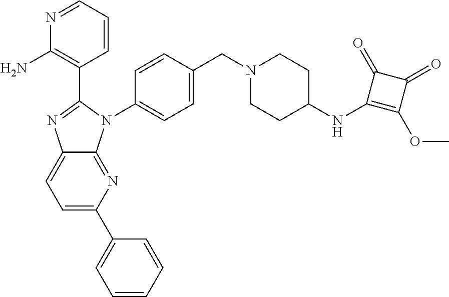 [SMILES: COc1c(NC2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzoyl)piperidin- 4-yl)amino)-4-methoxycyclobut-3- ene-1,2-dione |
| 29 | 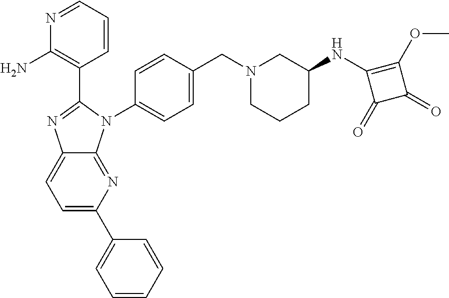 [SMILES: COc1c(N[C@H]2CCCN(Cc3ccc(-n4c(-c5cccnc5N)nc5ccc(-c6ccccc6)nc54)cc3)C2)c(=O)c1=O] | (S)-3-((1-(4-(2-(2-aminopyridin-3- yl)-5-phenyl-3H-imidazo[4,5- b]pyridin-3-yl)benzyl)piperidin-3- yl)amino)-4-methoxycyclobut-3- ene-1,2-dione |
| 30 | 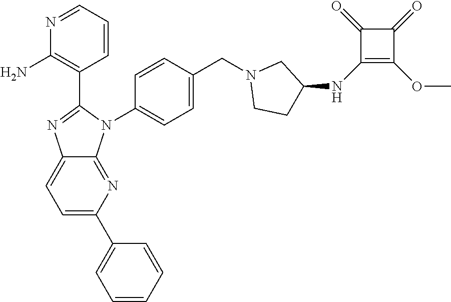 [SMILES: COc1c(N[C@H]2CCN(Cc3ccc(-n4c(-c5cccnc5N)nc5ccc(-c6ccccc6)nc54)cc3)C2)c(=O)c1=O] | (S)-3-((1-(4-(2-(2-aminopyridin-3- yl)-5-phenyl-3H-imidazo[4,5- b]pyridin-3-yl)benzyl)pyrrolidin- 3-yl)amino)-4-methoxycyclobut-3- ene-1,2-dione |
| 31 | 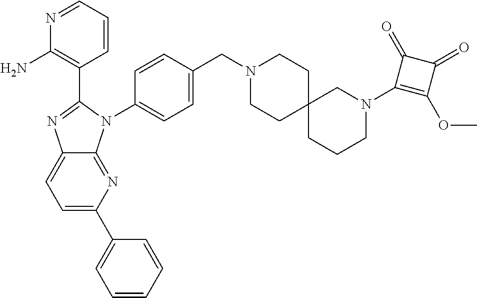 [SMILES: COc1c(N2CCCC3(CCN(Cc4ccc(-n5c(-c6cccnc6N)nc6ccc(-c7ccccc7)nc65)cc4)CC3)C2)c(=O)c1=O] | 3-(9-(4-(2-(2-aminopyridin-3-yl)- 5-phenyl-3H-imidazo[4,5- b]pyridin-3-yl)benzyl)-2,9- diazaspiro[5.5]undecan-2-yl)-4- methoxycyclobut-3-ene-1,2-dione |
| 32 | 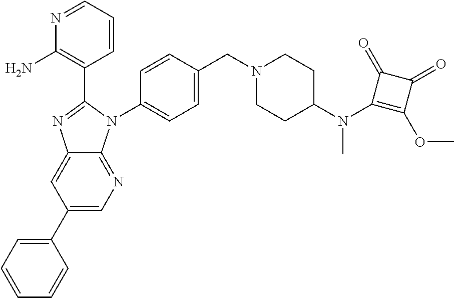 [SMILES: COc1c(N(C)C2CCN(Cc3ccc(-n4c(-c5cccnc5N)nc5cc(-c6ccccc6)cnc54)cc3)CC2)c(=O)c1=O] | 3-((1-(4-(2-(2-aminopyridin-3-yl)- 6-phenyl-3H-imidazo[4,5- b]pyridin-3-yl)benzyl)piperidin-4- yl)(methyl)amino)-4- methoxycyclobut-3-ene-1,2-dione |
| 33 | 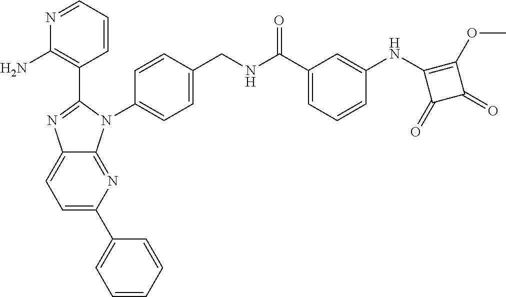 [SMILES: COc1c(Nc2cccc(C(=O)NCc3ccc(-n4c(-c5cccnc5N)nc5ccc(-c6ccccc6)nc54)cc3)c2)c(=O)c1=O] | N-(4-(2-(2-aminopyridin-3-yl)-5- phenyl-3H-imidazo[4,5-b]pyridin- 3-yl)benzyl)-3-((2-methoxy-3,4- dioxocyclobut-1-en-1- yl)amino)benzamide |
| 34 | 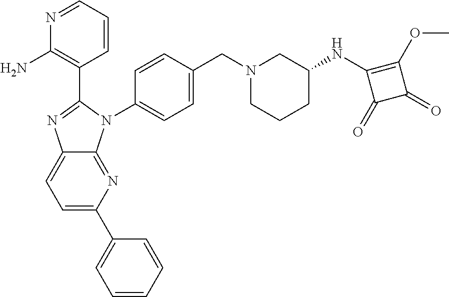 [SMILES: COc1c(N[C@@H]2CCCN(Cc3ccc(-n4c(-c5cccnc5N)nc5ccc(-c6ccccc6)nc54)cc3)C2)c(=O)c1=O] | (R)-3-((1-(4-(2-(2-aminopyridin-3- yl)-5-phenyl-3H-imidazo[4,5- b]pyridin-3-yl)benzyl)piperidin-3- yl)amino)-4-methoxycyclobut-3- ene-1,2-dione |
| 35 | 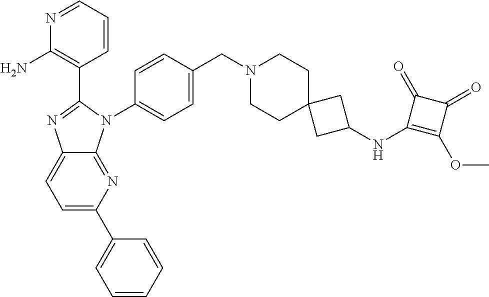 [SMILES: COc1c(NC2CC3(CCN(Cc4ccc(-n5c(-c6cccnc6N)nc6ccc(-c7ccccc7)nc65)cc4)CC3)C2)c(=O)c1=O] | 3-((7-(4-(2-(2-aminopyridin-3-yl)- 5-phenyl-3H-imidazo[4,5- b]pyridin-3-yl)benzyl)-7- azaspiro[3.5]nonan-2-yl)amino)-4- methoxycyclobut-3-ene-1,2-dione |
| 36 | 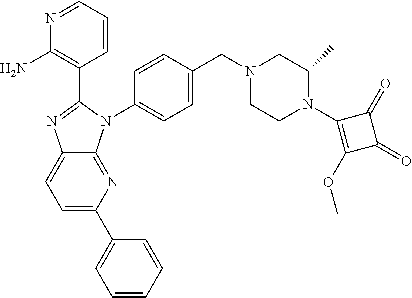 [SMILES: COc1c(N2CCN(Cc3ccc(-n4c(-c5cccnc5N)nc5ccc(-c6ccccc6)nc54)cc3)C[C@@H]2C)c(=O)c1=O] | (S)-3-(4-(4-(2-(2-aminopyridin-3- yl)-5-phenyl-3H-imidazo[4,5- b]pyridin-3-yl)benzyl)-2- methylpiperazin-1-yl)-4- methoxycyclobut-3-ene-1,2-dione |
| 37 | 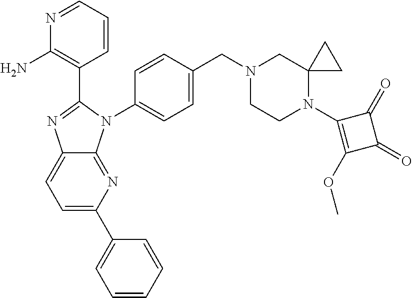 [SMILES: COc1c(N2CCN(Cc3ccc(-n4c(-c5cccnc5N)nc5ccc(-c6ccccc6)nc54)cc3)CC23CC3)c(=O)c1=O] | 3-(7-(4-(2-(2-aminopyridin-3-yl)- 5-phenyl-3H-imidazo[4,5- b]pyridin-3-yl)benzyl)-4,7- diazaspiro[2.5]octan-4-yl)-4- methoxycyclobut-3-ene-1,2-dione |
| 38 | 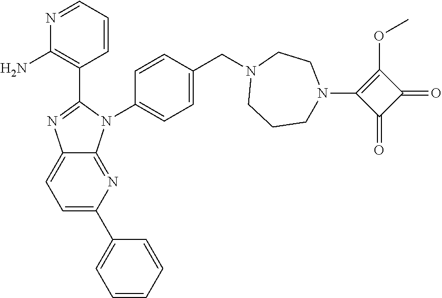 [SMILES: COc1c(N2CCCN(Cc3ccc(-n4c(-c5cccnc5N)nc5ccc(-c6ccccc6)nc54)cc3)CC2)c(=O)c1=O] | 3-(4-(4-(2-(2-aminopyridin-3-yl)- 5-phenyl-3H-imidazo[4,5- b]pyridin-3-yl)benzyl)-1,4- diazepan-1-yl)-4- methoxycyclobut-3-ene-1,2-dione |
| 39 | 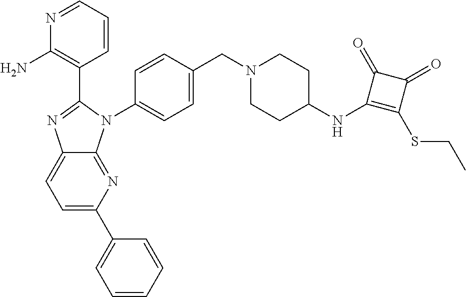 [SMILES: CCSc1c(NC2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)amino)-4-(ethylthio)cyclobut-3- ene-1,2-dione |
| 40 | 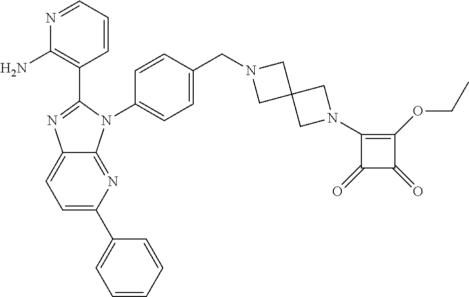 [SMILES: CCOc1c(N2CC3(CN(Cc4ccc(-n5c(-c6cccnc6N)nc6ccc(-c7ccccc7)nc65)cc4)C3)C2)c(=O)c1=O] | 3-(6-(4-(2-(2-aminopyridin-3-yl)- 5-phenyl-3H-imidazo[4,5- b]pyridin-3-yl)benzyl)-2,6- diazaspiro[3.3]heptan-2-yl)-4- ethoxycyclobut-3-ene-1,2-dione |
| 41 | 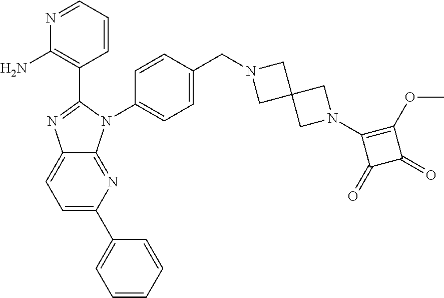 [SMILES: COc1c(N2CC3(CN(Cc4ccc(-n5c(-c6cccnc6N)nc6ccc(-c7ccccc7)nc65)cc4)C3)C2)c(=O)c1=O] | 3-(6-(4-(2-(2-aminopyridin-3-yl)- 5-phenyl-3H-imidazo[4,5- b]pyridin-3-yl)benzyl)-2,6- diazaspiro[3.3]heptan-2-yl)-4- methoxycyclobut-3-ene-1,2-dione |
| 42 | 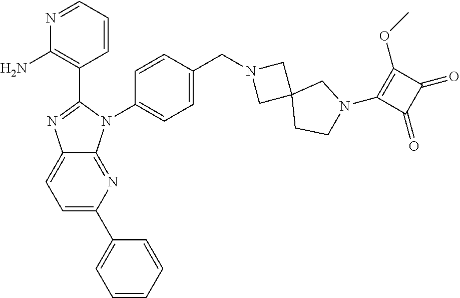 [SMILES: COc1c(N2CCC3(CN(Cc4ccc(-n5c(-c6cccnc6N)nc6ccc(-c7ccccc7)nc65)cc4)C3)C2)c(=O)c1=O] | 3-(2-(4-(2-(2-aminopyridin-3-yl)- 5-phenyl-3H-imidazo[4,5- b]pyridin-3-yl)benzyl)-2,6- diazaspiro[3.4]octan-6-yl)-4- methoxycyclobut-3-ene-1,2-dione |
| 43 | 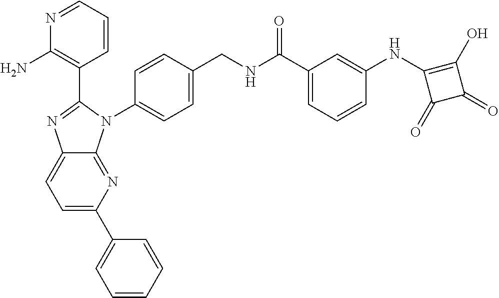 [SMILES: Nc1ncccc1-c1nc2ccc(-c3ccccc3)nc2n1-c1ccc(CNC(=O)c2cccc(Nc3c(O)c(=O)c3=O)c2)cc1] | N-(4-(2-(2-aminopyridin-3-yl)-5- phenyl-3H-imidazo[4,5-b]pyridin- 3-yl)benzyl)-3-((2-hydroxy-3,4- dioxocyclobut-1-en-1- yl)amino)benzamide |
| 44 | 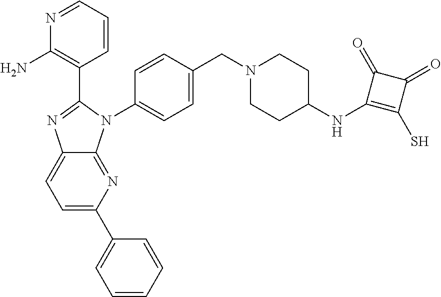 [SMILES: Nc1ncccc1-c1nc2ccc(-c3ccccc3)nc2n1-c1ccc(CN2CCC(Nc3c(S)c(=O)c3=O)CC2)cc1] | 3-((1-(4-(2-(2-aminopyridin-3-yl)- 5-phenyl-3H-imidazo[4,5- b]pyridin-3-yl)benzyl)piperidin-4- yl)amino)-4-mercaptocyclobut-3- ene-1,2-dione |
| 45 | 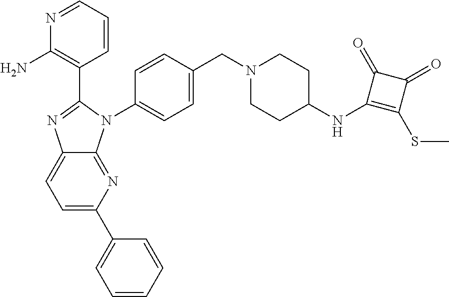 [SMILES: CSc1c(NC2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)amino)-4-(methylthio)cyclobut- 3-ene-1,2-dione |
| 46 | 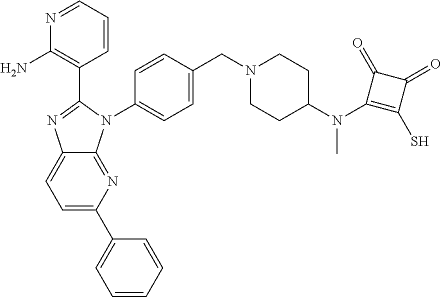 [SMILES: CN(c1c(S)c(=O)c1=O)C1CCN(Cc2ccc(-n3c(-c4cccnc4N)nc4ccc(-c5ccccc5)nc43)cc2)CC1] | 3-((1-(4-(2-(2-aminopyridin-3-yl)- 5-phenyl-3H-imidazo[4,5- b]pyridin-3-yl)benzyl)piperidin-4- yl)(methyl)amino)-4- mercaptocyclobut-3-ene-1,2-dione |
| 47 | 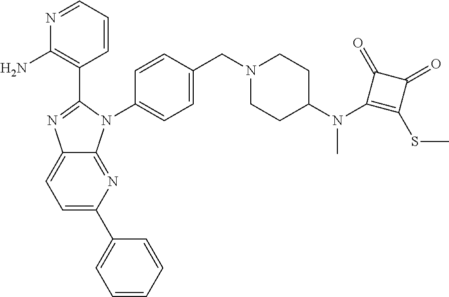 [SMILES: CSc1c(N(C)C2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)(methyl)amino)-4- (methylthio)cyclobut-3-ene-1,2- dione |
| 48 | 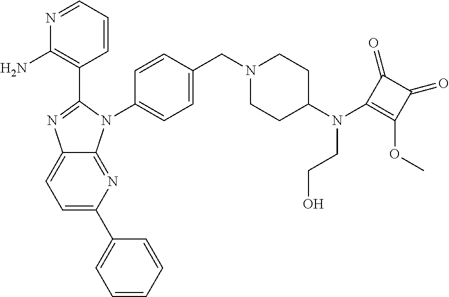 [SMILES: COc1c(N(CCO)C2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)(2-hydroxyethyl)amino)-4- methoxycyclobut-3-ene-1,2-dione |
| 49 | 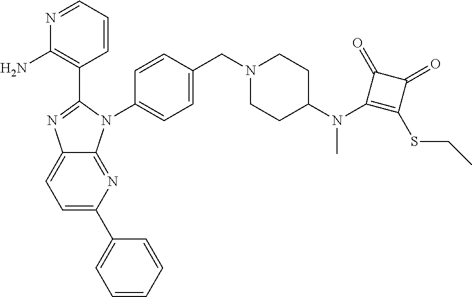 [SMILES: CCSc1c(N(C)C2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)(methyl)amino)-4- (ethylthio)cyclobut-3-ene-1,2- dione |
| 50 | 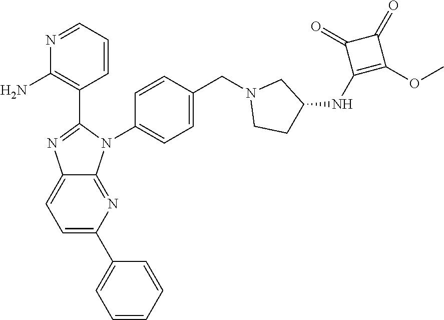 [SMILES: COc1c(N[C@@H]2CCN(Cc3ccc(-n4c(-c5cccnc5N)nc5ccc(-c6ccccc6)nc54)cc3)C2)c(=O)c1=O] | (R)-3-((1-(4-(2-(2-aminopyridin-3- yl)-5-phenyl-3H-imidazo[4,5- b]pyridin-3-yl)benzyl)pyrrolidin- 3-yl)amino)-4-methoxycyclobut-3- ene-1,2-dione |
| 51 | 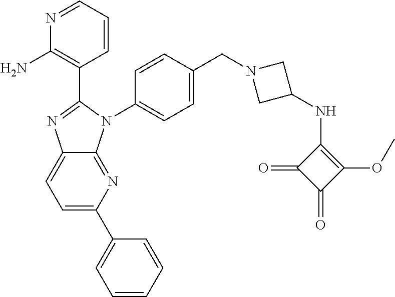 [SMILES: COc1c(NC2CN(Cc3ccc(-n4c(-c5cccnc5N)nc5ccc(-c6ccccc6)nc54)cc3)C2)c(=O)c1=O] | 3-((1-(4-(2-(2-aminopyridin-3-yl)- 5-phenyl-3H-imidazo[4,5- b]pyridin-3-yl)benzyl)azetidin-3- yl)amino)-4-methoxycyclobut-3- ene-1,2-dione |
| 52 | 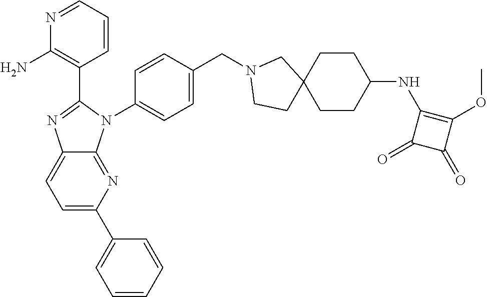 [SMILES: COc1c(NC2CCC3(CC2)CCN(Cc2ccc(-n4c(-c5cccnc5N)nc5ccc(-c6ccccc6)nc54)cc2)C3)c(=O)c1=O] | 3-((2-(4-(2-(2-aminopyridin-3-yl)- 5-phenyl-3H-imidazo[4,5- b]pyridin-3-yl)benzyl)-2- azaspiro[4.5]decan-8-yl)amino)-4- methoxycyclobut-3-ene-1,2-dione |
| 53 | 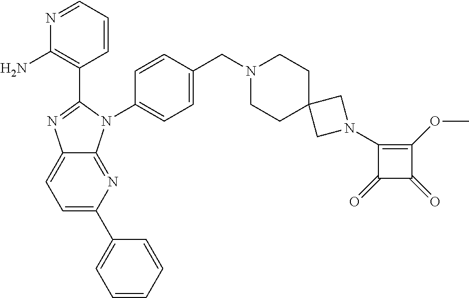 [SMILES: COc1c(N2CC3(CCN(Cc4ccc(-n5c(-c6cccnc6N)nc6ccc(-c7ccccc7)nc65)cc4)CC3)C2)c(=O)c1=O] | 3-(7-(4-(2-(2-aminopyridin-3-yl)- 5-phenyl-3H-imidazo[4,5- b]pyridin-3-yl)benzyl)-2,7- diazaspiro[3.5]nonan-2-yl)-4- methoxycyclobut-3-ene-1,2-dione |
| 54 | 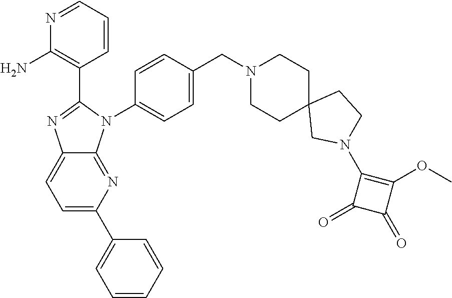 [SMILES: COc1c(N2CCC3(CCN(Cc4ccc(-n5c(-c6cccnc6N)nc6ccc(-c7ccccc7)nc65)cc4)CC3)C2)c(=O)c1=O] | 3-(8-(4-(2-(2-aminopyridin-3-yl)- 5-phenyl-3H-imidazo[4,5- b]pyridin-3-yl)benzyl)-2,8- diazaspiro[4.5]decan-2-yl)-4- methoxycyclobut-3-ene-1,2-dione |
| 55 | 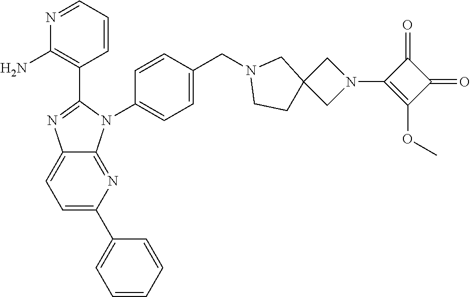 [SMILES: COc1c(N2CC3(CCN(Cc4ccc(-n5c(-c6cccnc6N)nc6ccc(-c7ccccc7)nc65)cc4)C3)C2)c(=O)c1=O] | 3-(6-(4-(2-(2-aminopyridin-3-yl)- 5-phenyl-3H-imidazo[4,5- b]pyridin-3-yl)benzyl)-2,6- diazaspiro[3.4]octan-2-yl)-4- methoxycyclobut-3-ene-1,2-dione |
| 56 | 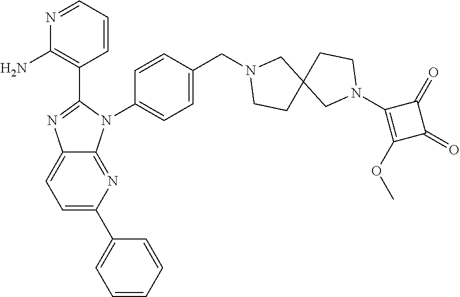 [SMILES: COc1c(N2CCC3(CCN(Cc4ccc(-n5c(-c6cccnc6N)nc6ccc(-c7ccccc7)nc65)cc4)C3)C2)c(=O)c1=O] | 3-(7-(4-(2-(2-aminopyridin-3-yl)- 5-phenyl-3H-imidazo[4,5- b]pyridin-3-yl)benzyl)-2,7- diazaspiro[4.4]nonan-2-yl)-4- methoxycyclobut-3-ene-1,2-dione |
| 57 | 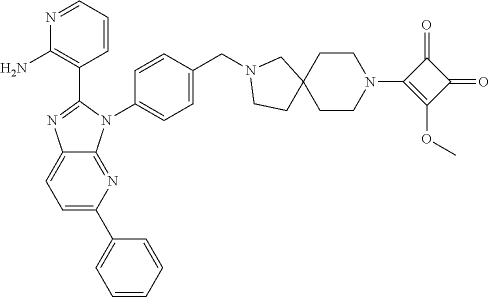 [SMILES: COc1c(N2CCC3(CCN(Cc4ccc(-n5c(-c6cccnc6N)nc6ccc(-c7ccccc7)nc65)cc4)C3)CC2)c(=O)c1=O] | 3-(2-(4-(2-(2-aminopyridin-3-yl)- 5-phenyl-3H-imidazo[4,5- b]pyridin-3-yl)benzyl)-2,8- diazaspiro[4.5]decan-8-yl)-4- methoxycyclobut-3-ene-1,2-dione |
| 58 | 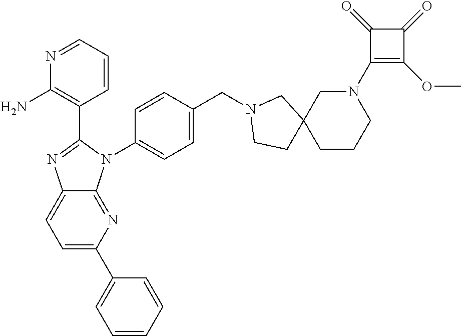 [SMILES: COc1c(N2CCCC3(CCN(Cc4ccc(-n5c(-c6cccnc6N)nc6ccc(-c7ccccc7)nc65)cc4)C3)C2)c(=O)c1=O] | 3-(2-(4-(2-(2-aminopyridin-3-yl)- 5-phenyl-3H-imidazo[4,5- b]pyridin-3-yl)benzyl)-2,7- diazaspiro[4.5]decan-7-yl)-4- methoxycyclobut-3-ene-1,2-dione |
| 59 | 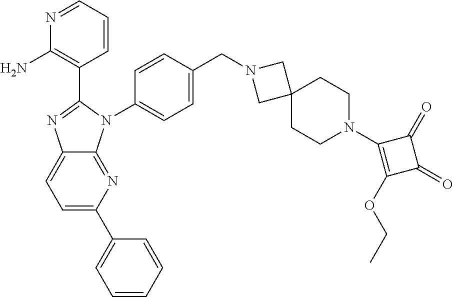 [SMILES: CCOc1c(N2CCC3(CC2)CN(Cc2ccc(-n4c(-c5cccnc5N)nc5ccc(-c6ccccc6)nc54)cc2)C3)c(=O)c1=O] | 3-(2-(4-(2-(2-aminopyridin-3-yl)- 5-phenyl-3H-imidazo[4,5- b]pyridin-3-yl)benzyl)-2,7- diazaspiro[3.5]nonan-7-yl)-4- ethoxycyclobut-3-ene-1,2-dione |
| 60 | 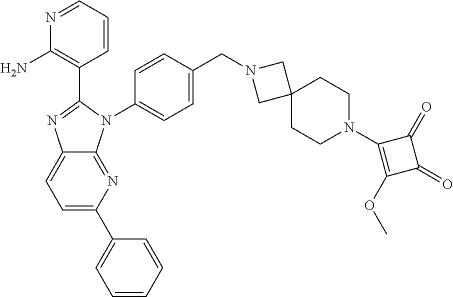 [SMILES: COc1c(N2CCC3(CC2)CN(Cc2ccc(-n4c(-c5cccnc5N)nc5ccc(-c6ccccc6)nc54)cc2)C3)c(=O)c1=O] | 3-(2-(4-(2-(2-aminopyridin-3-yl)- 5-phenyl-3H-imidazo[4,5- b]pyridin-3-yl)benzyl)-2,7- diazaspiro[3.5]nonan-7-yl)-4- methoxycyclobut-3-ene-1,2-dione |
| 61 | 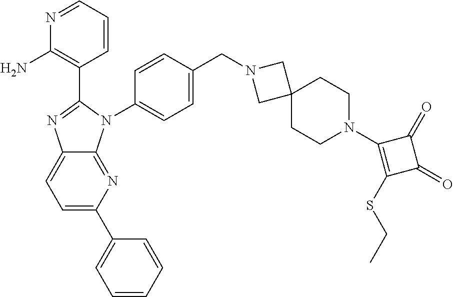 [SMILES: CCSc1c(N2CCC3(CC2)CN(Cc2ccc(-n4c(-c5cccnc5N)nc5ccc(-c6ccccc6)nc54)cc2)C3)c(=O)c1=O] | 3-(2-(4-(2-(2-aminopyridin-3-yl)- 5-phenyl-3H-imidazo[4,5- b]pyridin-3-yl)benzyl)-2,7- diazaspiro[3.5]nonan-7-yl)-4- (ethylthio)cyclobut-3-ene-1,2- dione |
| 62 | 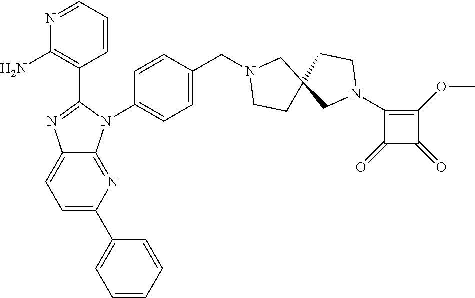 | (R)-3-(7-(4-(2-(2-aminopyridin-3- yl)-5-phenyl-3H-imidazo[4,5- b]pyridin-3-yl)benzyl)-2,7- diazaspiro[4.4]nonan-2-yl)-4- methoxycyclobut-3-ene-1,2-dione |
| 63 | 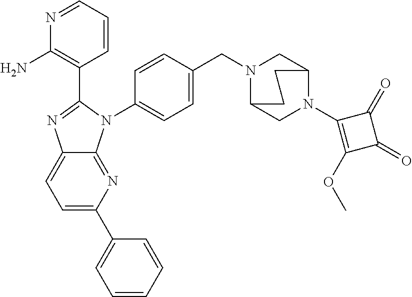 [SMILES: COc1c(N2CC3CCC2CN3Cc2ccc(-n3c(-c4cccnc4N)nc4ccc(-c5ccccc5)nc43)cc2)c(=O)c1=O] | 3-(5-(4-(2-(2-aminopyridin-3-yl)- 5-phenyl-3H-imidazo[4,5- b]pyridin-3-yl)benzyl)-2,5- diazabicyclo[2.2.2]octan-2-yl)-4- methoxycyclobut-3-ene-1,2-dione |
| 64 | 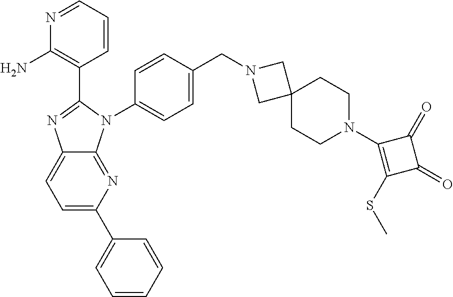 [SMILES: CSc1c(N2CCC3(CC2)CN(Cc2ccc(-n4c(-c5cccnc5N)nc5ccc(-c6ccccc6)nc54)cc2)C3)c(=O)c1=O] | 3-(2-(4-(2-(2-aminopyridin-3-yl)- 5-phenyl-3H-imidazo[4,5- b]pyridin-3-yl)benzyl)-2,7- diazaspiro[3.5]nonan-7-yl)-4- (methylthio)cyclobut-3-ene-1,2- dione |
| 65 | 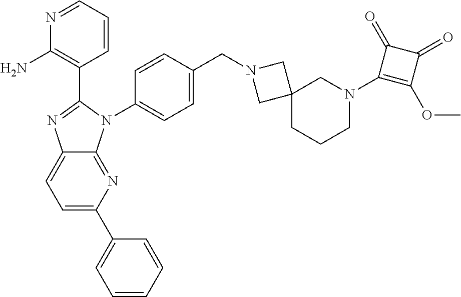 [SMILES: COc1c(N2CCCC3(CN(Cc4ccc(-n5c(-c6cccnc6N)nc6ccc(-c7ccccc7)nc65)cc4)C3)C2)c(=O)c1=O] | 3-(2-(4-(2-(2-aminopyridin-3-yl)- 5-phenyl-3H-imidazo[4,5- b]pyridin-3-yl)benzyl)-2,6- diazaspiro[3.5]nonan-6-yl)-4- methoxycyclobut-3-ene-1,2-dione |
| 66 | 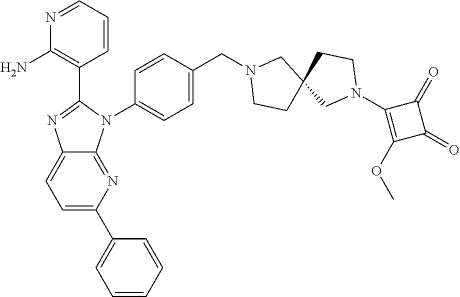 [SMILES: COc1c(N2CC[C@]3(CCN(Cc4ccc(-n5c(-c6cccnc6N)nc6ccc(-c7ccccc7)nc65)cc4)C3)C2)c(=O)c1=O] | (S)-3-(7-(4-(2-(2-aminopyridin-3- yl)-5-phenyl-3H-imidazo[4,5- b]pyridin-3-yl)benzyl)-2,7- diazaspiro[4.4]nonan-2-yl)-4- methoxycyclobut-3-ene-1,2-dione |
| 67 | 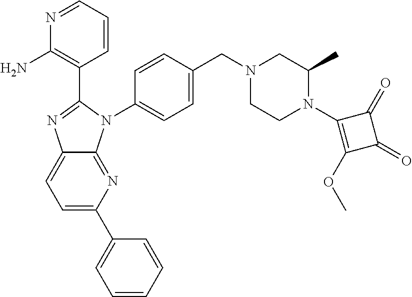 [SMILES: COc1c(N2CCN(Cc3ccc(-n4c(-c5cccnc5N)nc5ccc(-c6ccccc6)nc54)cc3)C[C@H]2C)c(=O)c1=O] | (R)-3-(4-(4-(2-(2-aminopyridin-3- yl)-5-phenyl-3H-imidazo[4,5- b]pyridin-3-yl)benzyl)-2- methylpiperazin-1-yl)-4- methoxycyclobut-3-ene-1,2-dione |
| 68 | 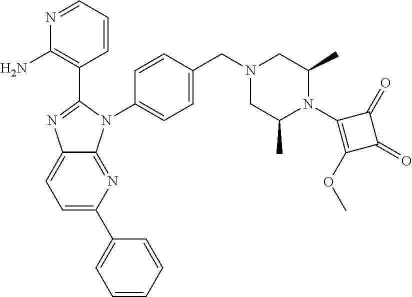 [SMILES: COc1c(N2[C@H](C)CN(Cc3ccc(-n4c(-c5cccnc5N)nc5ccc(-c6ccccc6)nc54)cc3)C[C@@H]2C)c(=O)c1=O] | 3-((2S,6R)-4-(4-(2-(2- aminopyridin-3-yl)-5-phenyl-3H- imidazo[4,5-b]pyridin-3- yl)benzyl)-2,6-dimethylpiperazin- 1-yl)-4-methoxycyclobut-3-ene- 1,2-dione |
| 69 | 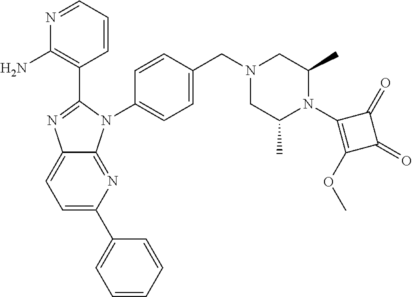 [SMILES: COc1c(N2[C@H](C)CN(Cc3ccc(-n4c(-c5cccnc5N)nc5ccc(-c6ccccc6)nc54)cc3)C[C@H]2C)c(=O)c1=O] | 3-((2R,6R)-4-(4-(2-(2- aminopyridin-3-yl)-5-phenyl-3H- imidazo[4,5-b]pyridin-3- yl)benzyl)-2,6-dimethylpiperazin- 1-yl)-4-methoxycyclobut-3-ene- 1,2-dione |
| 70 | 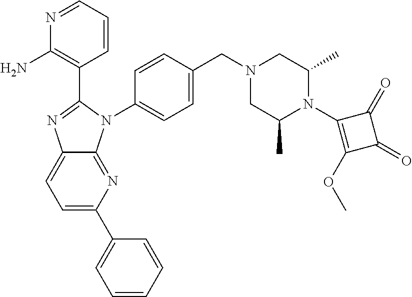 [SMILES: COc1c(N2[C@@H](C)CN(Cc3ccc(-n4c(-c5cccnc5N)nc5ccc(-c6ccccc6)nc54)cc3)C[C@@H]2C)c(=O)c1=O] | 3-((2S,6S)-4-(4-(2-(2- aminopyridin-3-yl)-5-phenyl-3H- imidazo[4,5-b]pyridin-3- yl)benzyl)-2,6-dimethylpiperazin- 1-yl)-4-methoxycyclobut-3-ene- 1,2-dione |
| 71 | 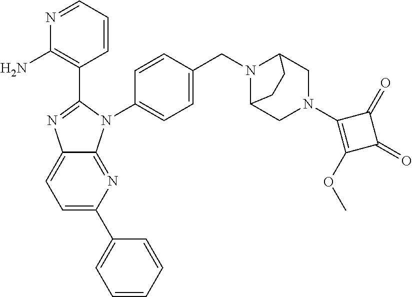 [SMILES: COc1c(N2CC3CCC(C2)N3Cc2ccc(-n3c(-c4cccnc4N)nc4ccc(-c5ccccc5)nc43)cc2)c(=O)c1=O] | 3-(8-(4-(2-(2-aminopyridin-3-yl)- 5-phenyl-3H-imidazo[4,5- b]pyridin-3-yl)benzyl)-3,8- diazabicyclo[3.2.1]octan-3-yl)-4- methoxycyclobut-3-ene-1,2-dione |
| 72 | 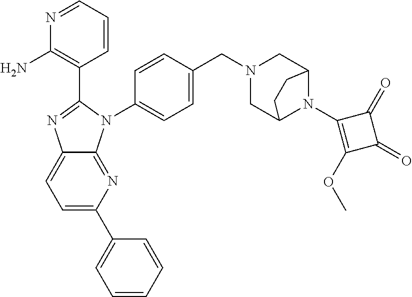 [SMILES: COc1c(N2C3CCC2CN(Cc2ccc(-n4c(-c5cccnc5N)nc5ccc(-c6ccccc6)nc54)cc2)C3)c(=O)c1=O] | 3-(3-(4-(2-(2-aminopyridin-3-yl)- 5-phenyl-3H-imidazo[4,5- b]pyridin-3-yl)benzyl)-3,8- diazabicyclo[3.2.1]octan-8-yl)-4- methoxycyclobut-3-ene-1,2-dione |
| 73 | 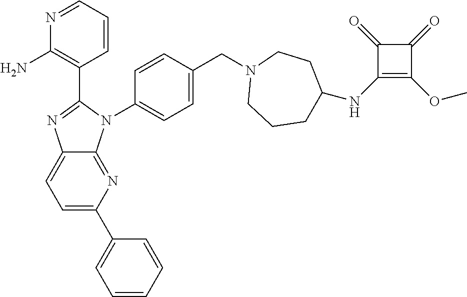 [SMILES: COc1c(NC2CCCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)azepan-4- yl)amino)-4-methoxycyclobut-3- ene-1,2-dione |
| 74 | 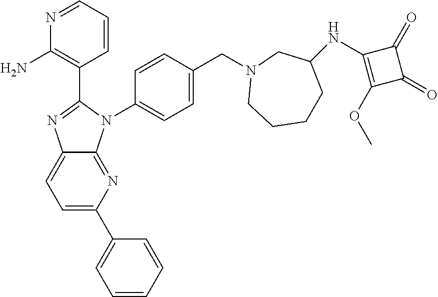 [SMILES: COc1c(NC2CCCCN(Cc3ccc(-n4c(-c5cccnc5N)nc5ccc(-c6ccccc6)nc54)cc3)C2)c(=O)c1=O] | 3-((1-(4-(2-(2-aminopyridin-3-yl)- 5-phenyl-3H-imidazo[4,5- b]pyridin-3-yl)benzyl)azepan-3- yl)amino)-4-methoxycyclobut-3- ene-1,2-dione |
| 75 | 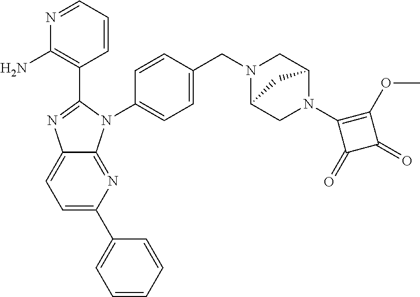 [SMILES: COc1c(N2C[C@@H]3C[C@H]2CN3Cc2ccc(-n3c(-c4cccnc4N)nc4ccc(-c5ccccc5)nc43)cc2)c(=O)c1=O] | 3-((1S,4S)-5-(4-(2-(2- aminopyridin-3-yl)-5-phenyl-3H- imidazo[4,5-b]pyridin-3- yl)benzyl)-2,5- diazabicyclo[2.2.1]heptan-2-y1)-4- methoxycyclobut-3-ene-1,2-dione |
| 76 | 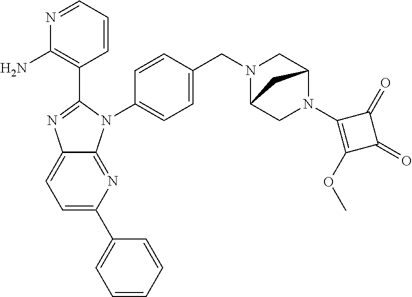 [SMILES: COc1c(N2C[C@H]3C[C@@H]2CN3Cc2ccc(-n3c(-c4cccnc4N)nc4ccc(-c5ccccc5)nc43)cc2)c(=O)c1=O] | 3-((1R,4R)-5-(4-(2-(2- aminopyridin-3-yl)-5-phenyl-3H- imidazo[4,5-b]pyridin-3- yl)benzyl)-2,5- diazabicyclo[2.2.1]heptan-2-yl)-4- methoxycyclobut-3-ene-1,2-dione |
| 77 | 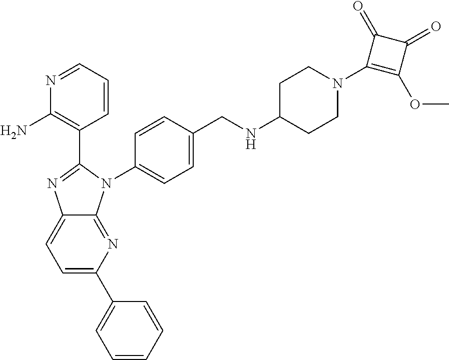 [SMILES: COc1c(N2CCC(NCc3ccc(-n4c(-c5cccnc5N)nc5ccc(-c6ccccc6)nc54)cc3)CC2)c(=O)c1=O] | 3-(4-((4-(2-(2-aminopyridin-3-yl)- 5-phenyl-3H-imidazo[4,5- b]pyridin-3- yl)benzyl)amino)piperidin-1-yl)-4- methoxycyclobut-3-ene-1,2-dione |
| 78 | 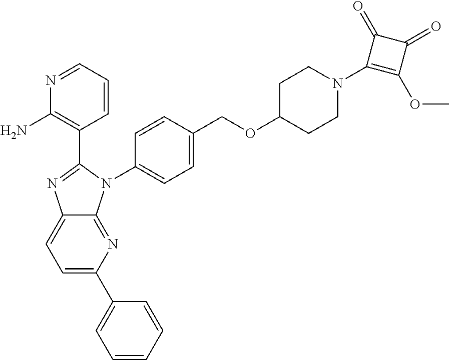 [SMILES: COc1c(N2CCC(OCc3ccc(-n4c(-c5cccnc5N)nc5ccc(-c6ccccc6)nc54)cc3)CC2)c(=O)c1=O] | 3-(4-((4-(2-(2-aminopyridin-3-yl)- 5-phenyl-3H-imidazo[4,5- b]pyridin-3- yl)benzyl)oxy)piperidin-1-yl)-4- methoxycyclobut-3-ene-1,2-dione |
| 79 | 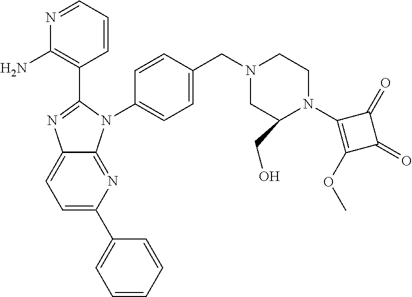 [SMILES: COc1c(N2CCN(Cc3ccc(-n4c(-c5cccnc5N)nc5ccc(-c6ccccc6)nc54)cc3)C[C@@H]2CO)c(=O)c1=O] | (R)-3-(4-(4-(2-(2-aminopyridin-3- yl)-5-phenyl-3H-imidazo[4,5- b]pyridin-3-yl)benzyl)-2- (hydroxymethyl)piperazin-1-yl)-4- methoxycyclobut-3-ene-1,2-dione |
| 80 | 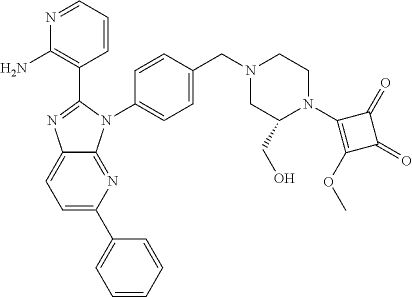 [SMILES: COc1c(N2CCN(Cc3ccc(-n4c(-c5cccnc5N)nc5ccc(-c6ccccc6)nc54)cc3)C[C@H]2CO)c(=O)c1=O] | (S)-3-(4-(4-(2-(2-aminopyridin-3- yl)-5-phenyl-3H-imidazo[4,5- b]pyridin-3-yl)benzyl)-2- (hydroxymethyl)piperazin-1-yl)-4- methoxycyclobut-3-ene-1,2-dione |
| 81 | 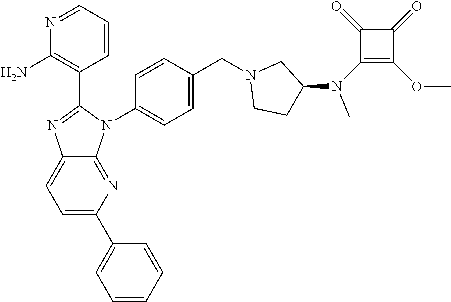 [SMILES: COc1c(N(C)[C@H]2CCN(Cc3ccc(-n4c(-c5cccnc5N)nc5ccc(-c6ccccc6)nc54)cc3)C2)c(=O)c1=O] | (S)-3-((1-(4-(2-(2-aminopyridin-3- yl)-5-phenyl-3H-imidazo[4,5- b]pyridin-3-yl)benzyl)pyrrolidin- 3-yl)(methyl)amino)-4- methoxycyclobut-3-ene-1,2-dione |
| 82 | 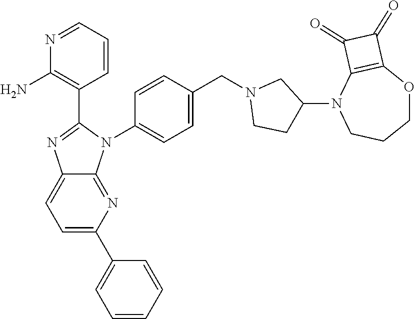 [SMILES: Nc1ncccc1-c1nc2ccc(-c3ccccc3)nc2n1-c1ccc(CN2CCC(N3CCCOc4c3c(=O)c4=O)C2)cc1] | 6-(1-(4-(2-(2-aminopyridin-3-yl)- 5-phenyl-3H-imidazo[4,5- b]pyridin-3-yl)benzyl)pyrrolidin- 3-yl)-2-oxa-6- azabicyclo[5.2.0]non-1(7)-ene-8,9- dione |
| 83 | 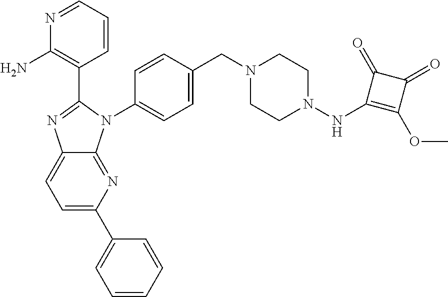 [SMILES: COc1c(NN2CCN(Cc3ccc(-n4c(-c5cccnc5N)nc5ccc(-c6ccccc6)nc54)cc3)CC2)c(=O)c1=O] | 3-((4-(4-(2-(2-aminopyridin-3-yl)- 5-phenyl-3H-imidazo[4,5- b]pyridin-3-yl)benzyl)piperazin-1- yl)amino)-4-methoxycyclobut-3- ene-1,2-dione |
| 84 | 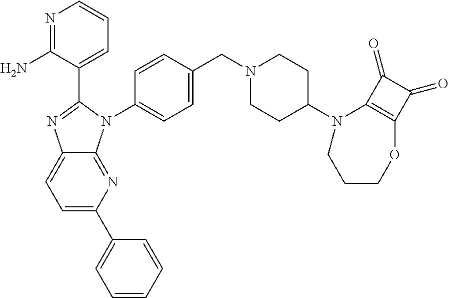 [SMILES: Nc1ncccc1-c1nc2ccc(-c3ccccc3)nc2n1-c1ccc(CN2CCC(N3CCCOc4c3c(=O)c4=O)CC2)cc1] | 6-(1-(4-(2-(2-aminopyridin-3-yl)- 5-phenyl-3H-imidazo[4,5- b]pyridin-3-yl)benzyl)piperidin-4- yl)-2-oxa-6-azabicyclo[5.2.0]non- 1(7)-ene-8,9-dione |
| 85 | 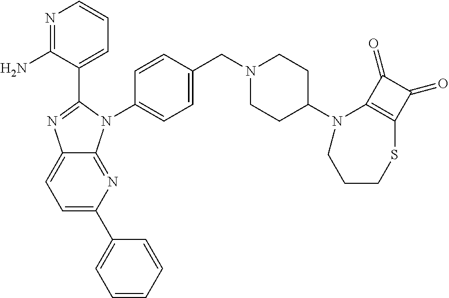 [SMILES: Nc1ncccc1-c1nc2ccc(-c3ccccc3)nc2n1-c1ccc(CN2CCC(N3CCCSc4c3c(=O)c4=O)CC2)cc1] | 6-(1-(4-(2-(2-aminopyridin-3-yl)- 5-phenyl-3H-imidazo[4,5- b]pyridin-3-yl)benzyl)piperidin-4- yl)-2-thia-6-azabicyclo[5.2.0]non- 1(7)-ene-8,9-dione |
| 86 | 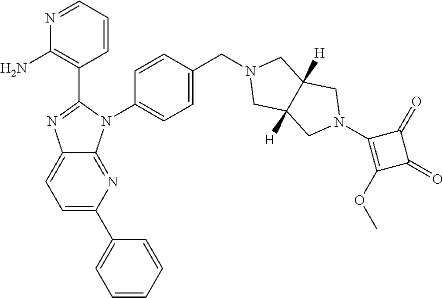 [SMILES: COc1c(N2C[C@H]3CN(Cc4ccc(-n5c(-c6cccnc6N)nc6ccc(-c7ccccc7)nc65)cc4)C[C@H]3C2)c(=O)c1=O] | 3-((3aR,6aS)-5-(4-(2-(2- aminopyridin-3-yl)-5-phenyl-3H- imidazo[4,5-b]pyridin-3- yl)benzyl)hexahydropyrrolo[3,4- c]pyrrol-2(1H)-yl)-4- methoxycyclobut-3-ene-1,2-dione |
| 87 | 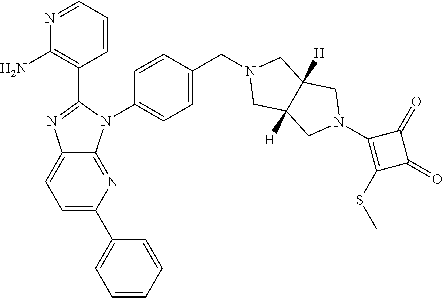 [SMILES: CSc1c(N2C[C@H]3CN(Cc4ccc(-n5c(-c6cccnc6N)nc6ccc(-c7ccccc7)nc65)cc4)C[C@H]3C2)c(=O)c1=O] | 3-((3aR,6aS)-5-(4-(2-(2- aminopyridin-3-yl)-5-phenyl-3H- imidazo[4,5-b]pyridin-3- yl)benzyl)hexahydropyrrolo[3,4- c]pyrrol-2(1H)-yl)-4- (methylthio)cyclobut-3-ene-1,2- dione |
| 88 | 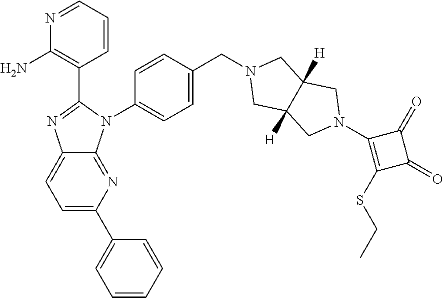 [SMILES: CCSc1c(N2C[C@H]3CN(Cc4ccc(-n5c(-c6cccnc6N)nc6ccc(-c7ccccc7)nc65)cc4)C[C@H]3C2)c(=O)c1=O] | 3-((3aR,6aS)-5-(4-(2-(2- aminopyridin-3-yl)-5-phenyl-3H- imidazo[4,5-b]pyridin-3- yl)benzyl)hexahydropyrrolo[3,4- c]pyrrol-2(1H)-yl)-4- (ethylthio)cyclobut-3-ene-1,2- dione |
| 89 | 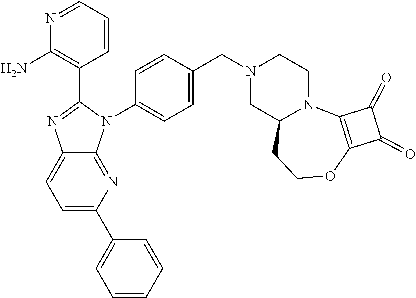 [SMILES: Nc1ncccc1-c1nc2ccc(-c3ccccc3)nc2n1-c1ccc(CN2CCN3c4c(c(=O)c4=O)OCC[C@H]3C2)cc1] | (S)-7-(4-(2-(2-aminopyridin-3-yl)- 5-phenyl-3H-imidazo[4,5- b]pyridin-3-yl)benzyl)- 5,5a,6,7,8,9-hexahydro-4H- cyclobuta[b]pyrazino[1,2- d][1,4]oxazepine-1,2-dione |
| 90 | 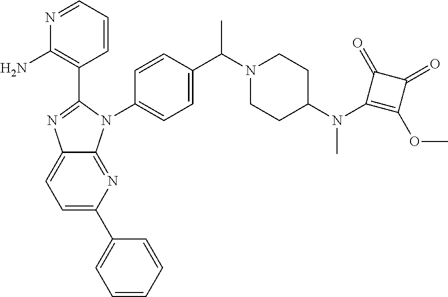 [SMILES: COc1c(N(C)C2CCN(C(C)c3ccc(-n4c(-c5cccnc5N)nc5ccc(-c6ccccc6)nc54)cc3)CC2)c(=O)c1=O] | 3-((1-(1-(4-(2-(2-aminopyridin-3- yl)-5-phenyl-3H-imidazo[4,5- b]pyridin-3- yl)phenyl)ethyl)piperidin-4- yl)(methyl)amino)-4- methoxycyclobut-3-ene-1,2-dione |
| 91 | 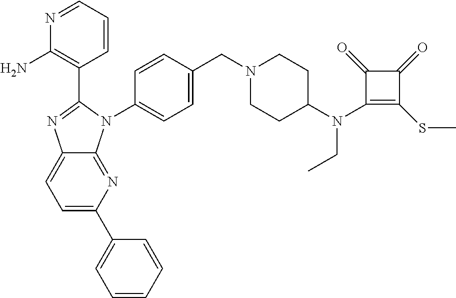 [SMILES: CCN(c1c(SC)c(=O)c1=O)C1CCN(Cc2ccc(-n3c(-c4cccnc4N)nc4ccc(-c5ccccc5)nc43)cc2)CC1] | 3-((1-(4-(2-(2-aminopyridin-3-yl)- 5-phenyl-3H-imidazo[4,5- b]pyridin-3-yl)benzyl)piperidin-4- yl)(ethyl)amino)-4- methoxycyclobut-3-ene-1,2-dione |
| 92 | 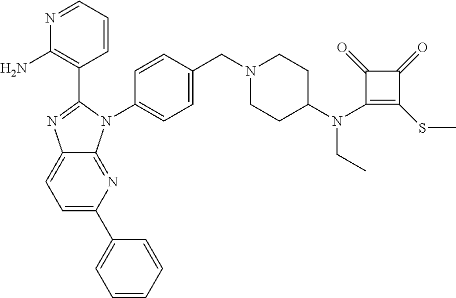 [SMILES: CCN(c1c(SC)c(=O)c1=O)C1CCN(Cc2ccc(-n3c(-c4cccnc4N)nc4ccc(-c5ccccc5)nc43)cc2)CC1] | 3-((1-(4-(2-(2-aminopyridin-3-yl)- 5-phenyl-3H-imidazo[4,5- b]pyridin-3-yl)benzyl)piperidin-4- yl)(ethyl)amino)-4- (methylthio)cyclobut-3-ene-1,2- dione |
| 93 | 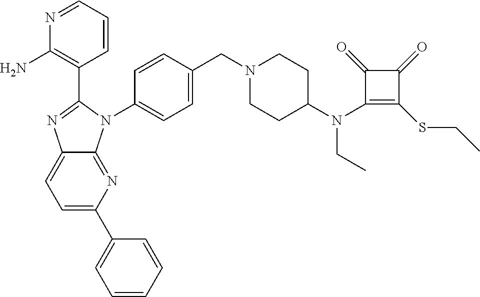 [SMILES: CCSc1c(N(CC)C2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)(ethyl)amino)-4- (ethylthio)cyclobut-3-ene-1,2- dione |
| 94 | 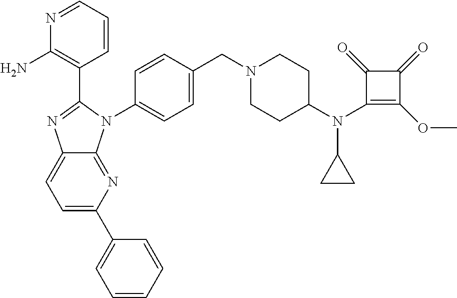 [SMILES: COc1c(N(C2CC2)C2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)(cyclopropyl)amino)-4- methoxycyclobut-3-ene-1,2-dione |
| 95 | 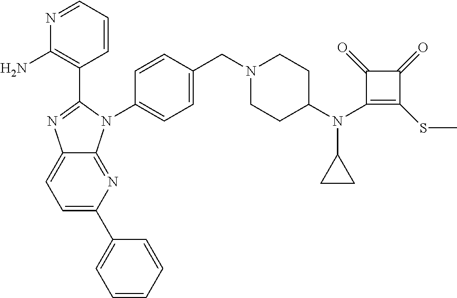 [SMILES: CSc1c(N(C2CC2)C2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzy1)piperidin-4- yl)(cyclopropyl)amino)-4- (methylthio)cyclobut-3-ene-1,2- dione |
| 96 | 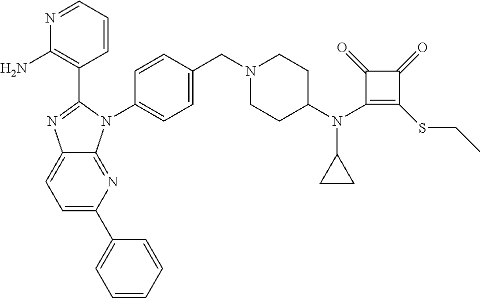 [SMILES: CCSc1c(N(C2CC2)C2CCN(Cc3ccc(-n4c(-c5cccnc5N)nc5ccc(-c6ccccc6)nc54)cc3)CC2)c(=O)c1=O] | 3-((1-(4-(2-(2-aminopyridin-3-yl)- 5-phenyl-3H-imidazo[4,5- b]pyridin-3-yl)benzyl)piperidin-4- yl)(cyclopropyl)amino)-4- (ethylthio)cyclobut-3-ene-1,2- dione |
| 97 | 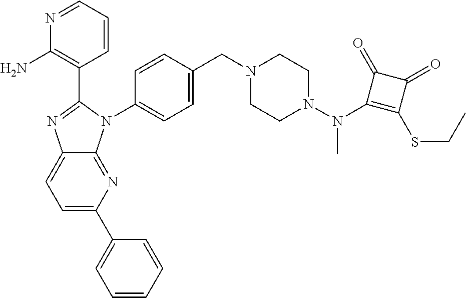 [SMILES: CCSc1c(N(C)N2CCN(Cc3ccc(-n4c(-c5cccnc5N)nc5ccc(-c6ccccc6)nc54)cc3)CC2)c(=O)c1=O] | 3-((4-(4-(2-(2-aminopyridin-3-yl)- 5-phenyl-3H-imidazo[4,5- b]pyridin-3-yl)benzyl)piperazin-1- yl)(methyl)amino)-4- (ethylthio)cyclobut-3-ene-1,2- dione |
| 98 | 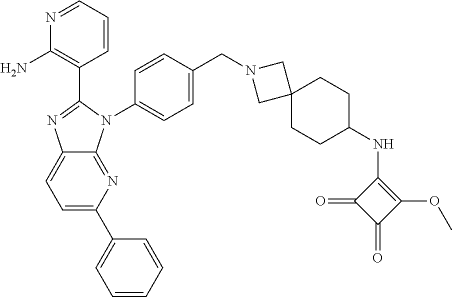 [SMILES: COc1c(NC2CCC3(CC2)CN(Cc2ccc(-n4c(-c5cccnc5N)nc5ccc(-c6ccccc6)nc54)cc2)C3)c(=O)c1=O] | 3-((2-(4-(2-(2-aminopyridin-3-yl)- 5-phenyl-3H-imidazo[4,5- b]pyridin-3-yl)benzyl)-2- azaspiro[3.5]nonan-7-yl)amino)-4- methoxycyclobut-3-ene-1,2-dione |
| 99 | 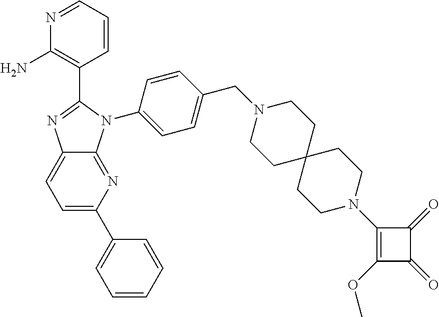 [SMILES: COc1c(N2CCC3(CCN(Cc4ccc(-n5c(-c6cccnc6N)nc6ccc(-c7ccccc7)nc65)cc4)CC3)CC2)c(=O)c1=O] | 3-(9-(4-(2-(2-aminopyridin-3-yl)- 5-phenyl-3H-imidazo[4,5- b]pyridin-3-yl)benzyl)-3,9- diazaspiro[5.5]undecan-3-yl)-4- methoxycyclobut-3-ene-1,2-dione |
| 100 | 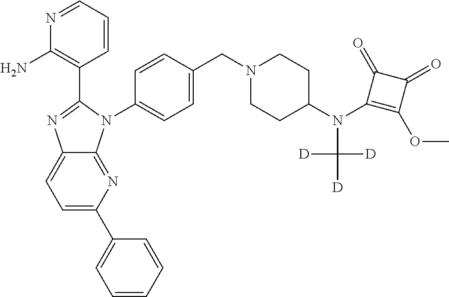 [SMILES: [2H]C([2H])([2H])N(c1c(OC)c(=O)c1=O)C1CCN(Cc2ccc(-n3c(-c4cccnc4N)nc4ccc(-c5ccccc5)nc43)cc2)CC1] | 3-((1-(4-(2-(2-aminopyridin-3-yl)- 5-phenyl-3H-imidazo[4,5- b]pyridin-3-yl)benzyl)piperidin-4- yl)(methyl-d3)amino)-4- methoxycyclobut-3-ene-1,2-dione |

Another embodiment provides an AKT1 inhibitory compound, or a pharmaceutically acceptable salt or solvate thereof, having a structure presented in Table 2.

TABLE 2

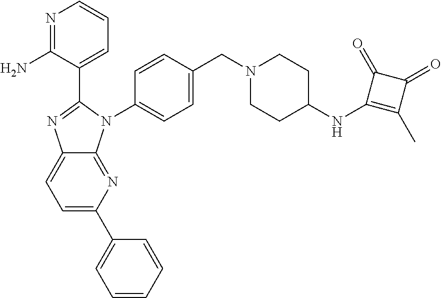 [SMILES: Cc1c(NC2CCN(Cc3ccc(-n4c(-c5cccnc5N)nc5ccc(-c6ccccc6)nc54)cc3)CC2)c(=O)c1=O]

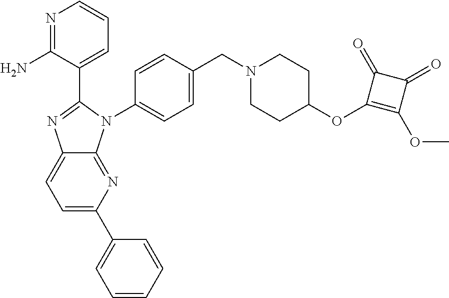 [SMILES: COc1c(OC2CCN(Cc3ccc(-n4c(-c5cccnc5N)nc5ccc(-c6ccccc6)nc54)cc3)CC2)c(=O)c1=O]

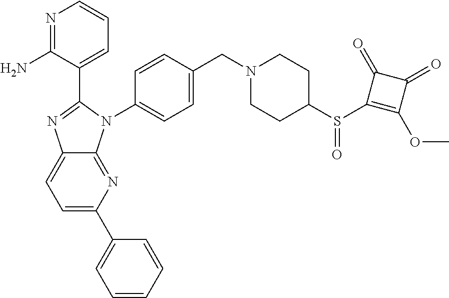 [SMILES: COc1c(S(=O)C2CCN(Cc3ccc(-n4c(-c5cccnc5N)nc5ccc(-c6ccccc6)nc54)cc3)CC2)c(=O)c1=O]

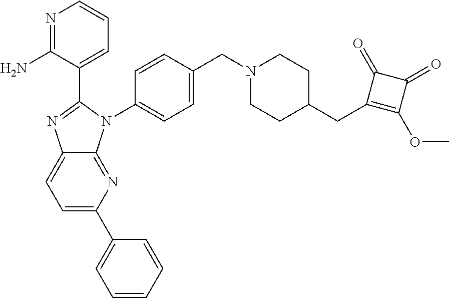 [SMILES: COc1c(CC2CCN(Cc3ccc(-n4c(-c5cccnc5N)nc5ccc(-c6ccccc6)nc54)cc3)CC2)c(=O)c1=O]

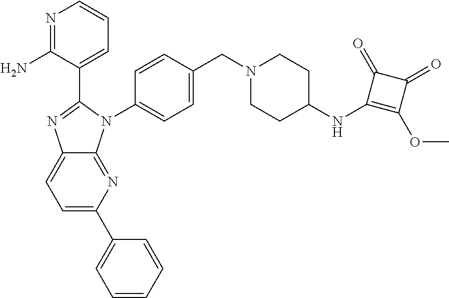 [SMILES: COc1c(NC2CCN(Cc3ccc(-n4c(-c5cccnc5N)nc5ccc(-c6ccccc6)nc54)cc3)CC2)c(=O)c1=O]

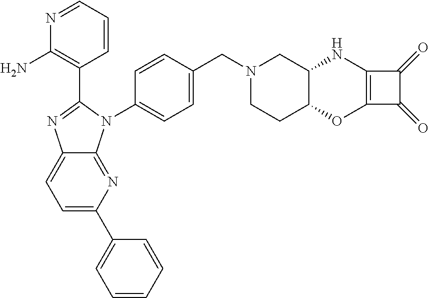 [SMILES: Nc1ncccc1-c1nc2ccc(-c3ccccc3)nc2n1-c1ccc(CN2CC[C@H]3Oc4c(c(=O)c4=O)N[C@H]3C2)cc1]

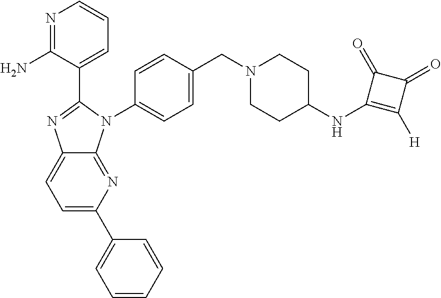 [SMILES: Nc1ncccc1-c1nc2ccc(-c3ccccc3)nc2n1-c1ccc(CN2CCC(Nc3cc(=O)c3=O)CC2)cc1]

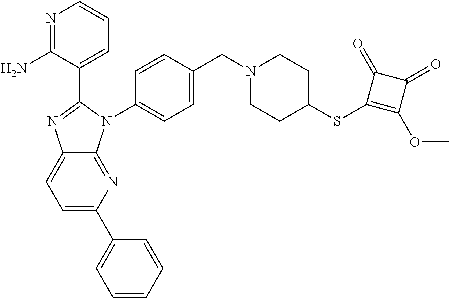 [SMILES: COc1c(SC2CCN(Cc3ccc(-n4c(-c5cccnc5N)nc5ccc(-c6ccccc6)nc54)cc3)CC2)c(=O)c1=O]

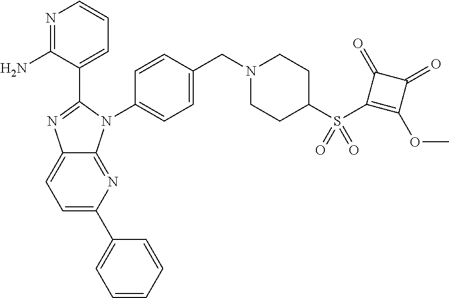 [SMILES: COc1c(S(=O)(=O)C2CCN(Cc3ccc(-n4c(-c5cccnc5N)nc5ccc(-c6ccccc6)nc54)cc3)CC2)c(=O)c1=O]

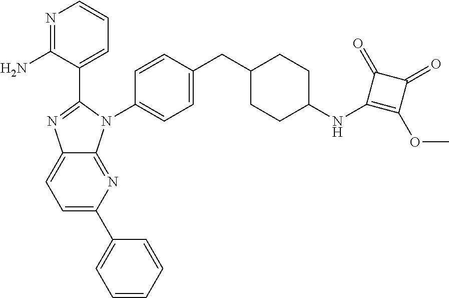 [SMILES: COc1c(NC2CCC(Cc3ccc(-n4c(-c5cccnc5N)nc5ccc(-c6ccccc6)nc54)cc3)CC2)c(=O)c1=O]

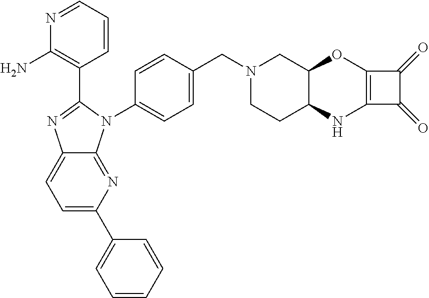 [SMILES: Nc1ncccc1-c1nc2ccc(-c3ccccc3)nc2n1-c1ccc(CN2CC[C@@H]3Nc4c(c(=O)c4=O)O[C@@H]3C2)cc1]

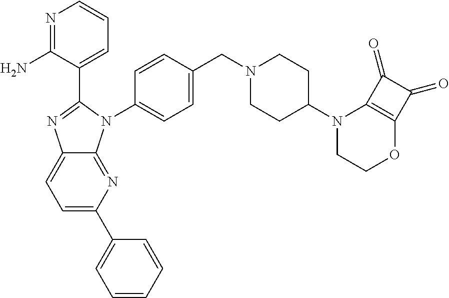 [SMILES: Nc1ncccc1-c1nc2ccc(-c3ccccc3)nc2n1-c1ccc(CN2CCC(N3CCOc4c3c(=O)c4=O)CC2)cc1]

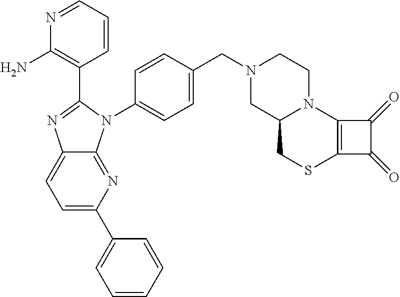 [SMILES: Nc1ncccc1-c1nc2ccc(-c3ccccc3)nc2n1-c1ccc(CN2CCN3c4c(c(=O)c4=O)SC[C@H]3C2)cc1]

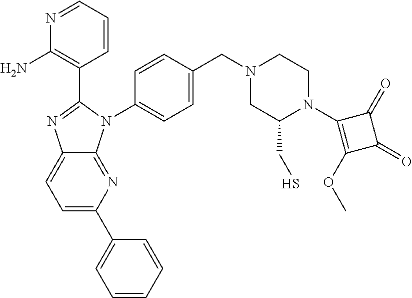 [SMILES: COc1c(N2CCN(Cc3ccc(-n4c(-c5cccnc5N)nc5ccc(-c6ccccc6)nc54)cc3)C[C@H]2CS)c(=O)c1=O]

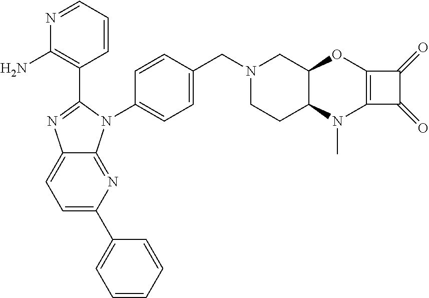 [SMILES: CN1c2c(c(=O)c2=O)O[C@@H]2CN(Cc3ccc(-n4c(-c5cccnc5N)nc5ccc(-c6ccccc6)nc54)cc3)CC[C@@H]21]

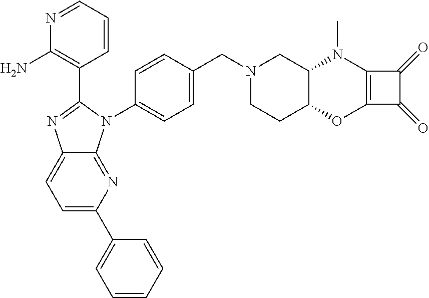 [SMILES: CN1c2c(c(=O)c2=O)O[C@@H]2CCN(Cc3ccc(-n4c(-c5cccnc5N)nc5ccc(-c6ccccc6)nc54)cc3)C[C@@H]21]

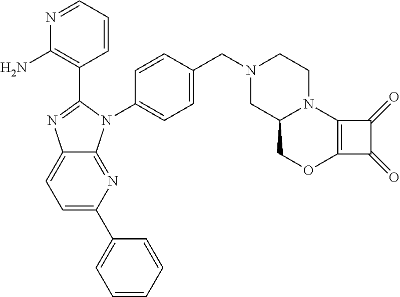 [SMILES: Nc1ncccc1-c1nc2ccc(-c3ccccc3)nc2n1-c1ccc(CN2CCN3c4c(c(=O)c4=O)OC[C@H]3C2)cc1]

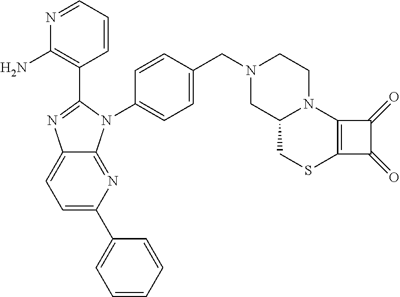 [SMILES: Nc1ncccc1-c1nc2ccc(-c3ccccc3)nc2n1-c1ccc(CN2CCN3c4c(c(=O)c4=O)SC[C@@H]3C2)cc1]

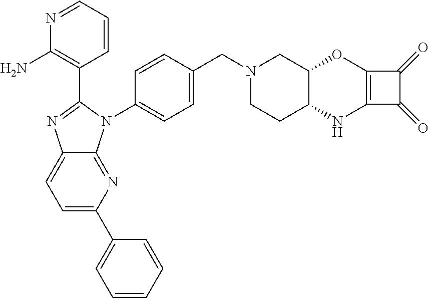 [SMILES: Nc1ncccc1-c1nc2ccc(-c3ccccc3)nc2n1-c1ccc(CN2CC[C@H]3Nc4c(c(=O)c4=O)O[C@H]3C2)cc1]

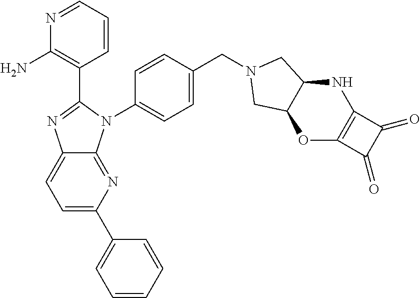 [SMILES: Nc1ncccc1-c1nc2ccc(-c3ccccc3)nc2n1-c1ccc(CN2C[C@@H]3Oc4c(c(=O)c4=O)N[C@@H]3C2)cc1]

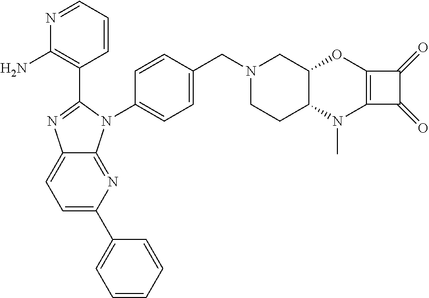 [SMILES: CN1c2c(c(=O)c2=O)O[C@H]2CN(Cc3ccc(-n4c(-c5cccnc5N)nc5ccc(-c6ccccc6)nc54)cc3)CC[C@H]21]

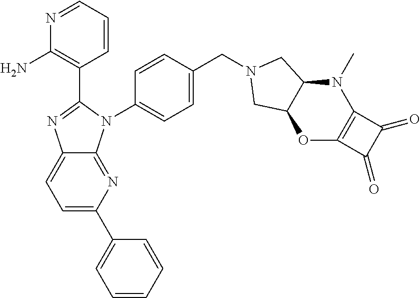 [SMILES: CN1c2c(c(=O)c2=O)O[C@H]2CN(Cc3ccc(-n4c(-c5cccnc5N)nc5ccc(-c6ccccc6)nc54)cc3)C[C@H]21]

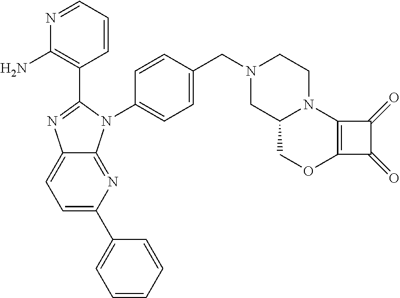 [SMILES: Nc1ncccc1-c1nc2ccc(-c3ccccc3)nc2n1-c1ccc(CN2CCN3c4c(c(=O)c4=O)OC[C@@H]3C2)cc1]

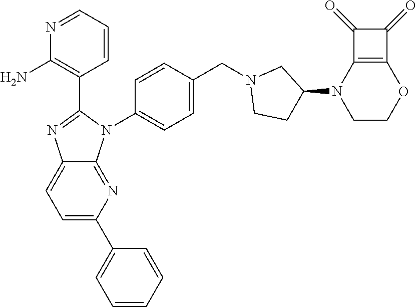 [SMILES: Nc1ncccc1-c1nc2ccc(-c3ccccc3)nc2n1-c1ccc(CN2CC[C@H](N3CCOc4c3c(=O)c4=O)C2)cc1]

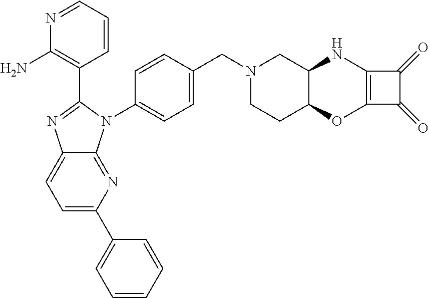 [SMILES: Nc1ncccc1-c1nc2ccc(-c3ccccc3)nc2n1-c1ccc(CN2CC[C@@H]3Oc4c(c(=O)c4=O)N[C@@H]3C2)cc1]

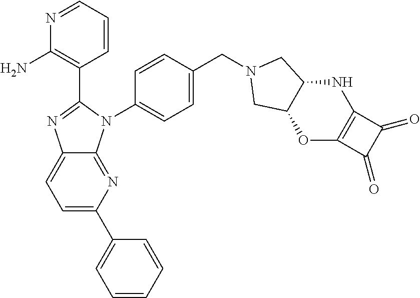 [SMILES: Nc1ncccc1-c1nc2ccc(-c3ccccc3)nc2n1-c1ccc(CN2C[C@@H]3Nc4c(c(=O)c4=O)O[C@@H]3C2)cc1]

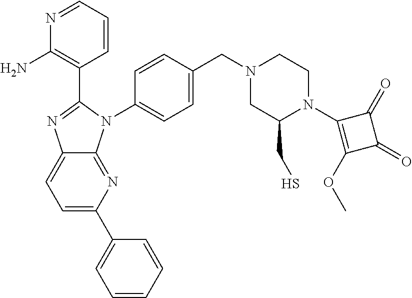 [SMILES: COc1c(N2CCN(Cc3ccc(-n4c(-c5cccnc5N)nc5ccc(-c6ccccc6)nc54)cc3)C[C@@H]2CS)c(=O)c1=O]

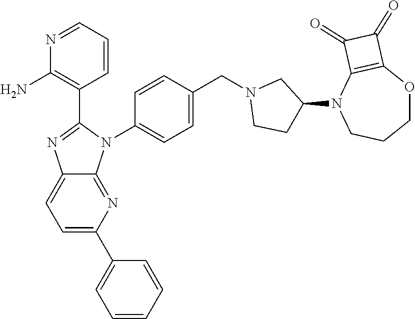 [SMILES: Nc1ncccc1-c1nc2ccc(-c3ccccc3)nc2n1-c1ccc(CN2CC[C@H](N3CCCOc4c3c(=O)c4=O)C2)cc1]

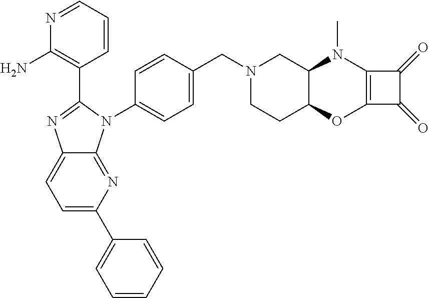 [SMILES: CN1c2c(c(=O)c2=O)O[C@H]2CCN(Cc3ccc(-n4c(-c5cccnc5N)nc5ccc(-c6ccccc6)nc54)cc3)C[C@H]21]

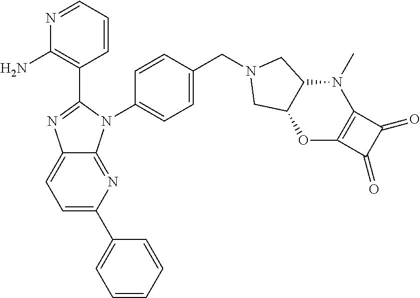

### Preparation of Compounds

The compounds used in the synthetic chemistry reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the AKT1 inhibitory compound described herein is administered as a pure chemical. In other embodiments, the AKT1 inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one AKT1 inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the AKT1 inhibitory compound as described by Formula (I), or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Table 1 or Table 2, or a pharmaceutically acceptable salt or solvate thereof.

One embodiment provides a method of preparing a pharmaceutical composition comprising mixing a compound of Table 1 or Table 2, or a pharmaceutically acceptable salt or solvate thereof, and a pharmaceutically acceptable carrier.

In certain embodiments, the AKT1 inhibitory compound as described by Table 1 or Table 2, or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the AKT1 inhibitory compound as described by Formula (I) or Table 1 or Table 2, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one AKT1 inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer or neoplastic disease.

One embodiment provides a pharmaceutical composition comprising a compound of Formula (I), or a pharmaceutically acceptable salt or solvate thereof, and a pharmaceutically acceptable excipient for use in a method of treatment of cancer or neoplastic disease.

One embodiment provides a use of a compound of Formula (I), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (I), or a pharmaceutically acceptable salt or solvate thereof. In some embodiments is provided a method of treating cancer, in a patient in need thereof, comprising administering to the patient a pharmaceutical composition comprising a compound of Formula (I), or a pharmaceutically acceptable salt or solvate thereof, and a pharmaceutically acceptable excipient.

One embodiment provides a compound of Table 1 or Table 2, or a pharmaceutically acceptable salt or solvate thereof, for use in a method of treatment of the human or animal body.

One embodiment provides a compound of Table 1 or Table 2, or a pharmaceutically acceptable salt or solvate thereof, for use in a method of treatment of cancer or neoplastic disease.

One embodiment provides a pharmaceutical composition comprising a compound of Table 1 or Table 2, or a pharmaceutically acceptable salt or solvate thereof, and a pharmaceutically acceptable excipient for use in a method of treatment of cancer or neoplastic disease.

One embodiment provides a use of a compound of Table 1 or Table 2, or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Table 1 or Table 2, or a pharmaceutically acceptable salt or solvate thereof. In some embodiments is provided a method of treating cancer, in a patient in need thereof, comprising administering to the patient a pharmaceutical composition comprising a compound of Table 1 or Table 2, or a pharmaceutically acceptable salt or solvate thereof, and a pharmaceutically acceptable excipient.

Provided herein is the method wherein the pharmaceutical composition is administered orally. Provided herein is the method wherein the pharmaceutical composition is administered by injection.

One embodiment provides a method of inhibiting a AKT1 enzyme comprising contacting the AKT1 enzyme with a compound of Formula (I) or Table 1 or Table 2. Another embodiment provides the method of inhibiting a AKT1 enzyme, wherein the AKT1 enzyme is contacted in an in vivo setting. Another embodiment provides the method of inhibiting a AKT1 enzyme, wherein the AKT1 enzyme is contacted in an in vitro setting.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the AKT1 inhibitory compounds disclosed herein are synthesized according to the following examples. As used below, and throughout the description of the invention, the following abbreviations, unless otherwise indicated, shall be understood to have the following meanings:
ACN acetonitrile
° C. degrees Celsius
δ_{H} chemical shift in parts per million downfield from tetramethylsilane
DCM dichloromethane (CH₂Cl₂)
DIAD diisopropyl azodicarboxylate
DIEA diisopropylethylamine
DMF dimethylformamide
DMSO dimethylsulfoxide
EA ethyl acetate
EtOAc ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MsCl methanesulfonyl chloride
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
SFC Supercritical fluid chromatography
T temperature
TFA trifluoroacetic acid
THF tetrahydrofuran
TPP Triphenylphosphine

### EXPERIMENTAL PROCEDURES

### Intermediate 1: 3-(3-(4-(Aminomethyl)phenyl)-3H-imidazo[4,5-b]pyridin-2-yl)pyridin-2-amine

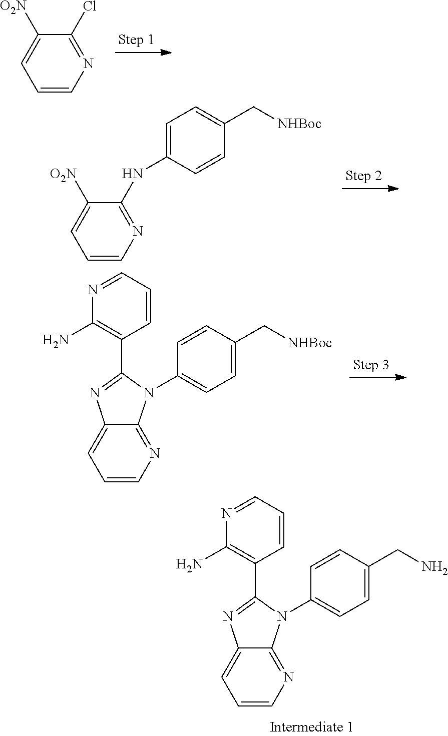

### Step 1: tert-Butyl 4-((3-nitropyridin-2-yl)amino)benzylcarbamate

To a solution of 2-chloro-3-nitro-pyridine (7.0 g, 44.2 mmol) and tert-butyl N-[(4-aminophenyl)methyl]carbamate (9.8 g, 44.2 mmol) in DMSO (100 mL) was added DIEA (11.4 g, 88.3 mmol). The mixture was stirred at 80° C. for 12 hr. The reaction mixture was diluted with H₂O (100 mL) at 25° C. and extracted with EtOAc (100 mL×3). The combined organic layers were washed with brine (150 mL), dried over Na₂SO₄, filtered, and concentrated under reduced pressure. The residue was triturated with (petroleum ether:EtOAc=10:1) to give tert-butyl N-[[4-[(3-nitro-2-pyridyl)amino]phenyl]methyl]carbamate (13.9 g, yield: 91%) as a red solid. MS: m/z=344.8 [M+H]⁺. ¹H NMR (400 MHz, Dimethylsulfoxide-d₆) δ 9.90 (s, 1H), 8.49 (dd, J=8.4, 1.6 Hz, 1H), 8.45 (dd, J=4.4, 1.6 Hz, 1H), 7.52 (d, J=8.4 Hz, 2H), 7.35 (t, J=6.0 Hz, 1H), 7.19 (d, J=8.4 Hz, 2H), 6.93 (dd, J=8.4, 4.4 Hz, 1H), 4.07 (d, J=6.0 Hz, 2H), 1.36 (s, 9H).

### Step 2: tert-Butyl 4-(2-(2-aminopyridin-3-yl)-3H-imidazo[4,5-b]pyridin-3-yl)benzylcarbamate

To a solution of tert-butyl N-[[4-[(3-nitro-2-pyridyl)amino]phenyl]methyl]carbamate (10 g, 29.0 mmol) in MeOH (70 mL) and DMSO (140 mL) were added 2-aminopyridine-3-carbaldehyde (3.9 g, 31.9 mmol) and Na₂S₂O₄ (10.1 g, 58.1 mmol). The mixture was stirred at 100° C. for 12 hr. After cooling to room temperature, the reaction mixture was diluted with H₂O (200 mL) and extracted with EtOAc (400 mL×2). The combined organic layers were washed with brine (400 mL), dried over Na₂SO₄, filtered, and concentrated under reduced pressure. The crude product was purified by silica gel flash chromatography (Eluent of 1~2% MeOH in CH₂Cl₂) to give tert-butyl 4-(2-(2-aminopyridin-3-yl)-3H-imidazo[4,5-b]pyridin-3-yl)benzylcarbamate (5.7 g, yield: 44%) as a red solid. MS: m/z=417.4 [M+H]⁺. ¹H NMR (400 MHz, Dimethylsulfoxide-d₆) δ 8.31 (dd, J=4.8, 1.2 Hz, 1H), 8.20 (dd, J=8.0, 1.2 Hz, 1H), 7.99 (dd, J=4.8, 1.6 Hz, 1H), 7.49 (t, J=6.0 Hz, 1H), 7.41-7.36 (m, 5H), 7.21 (dd, J=7.6, 1.6 Hz, 1H), 6.99 (br s, 2H), 6.39 (dd, J=7.6, 4.8 Hz, 1H), 4.21 (d, J=6.0 Hz, 2H), 1.41 (s, 9H).

### Step 3: 3-(3-(4-(Aminomethyl)phenyl)-3H-imidazo[4,5-b]pyridin-2-yl)pyridin-2-amine

To a solution of tert-butyl N-[[4-[2-(2-amino-3-pyridyl)imidazo[4,5-b]pyridin-3-yl]phenyl]methyl]carbamate (350 mg, 840 μmol) in 1,4-dioxane (3 mL) was added 4 M HCl in 1,4-dioxane (1 mL) at 20° C. The mixture was stirred at 20° C. for 2 hr. The reaction was concentrated under reduced pressure to give a crude product (280 mg, HCl salt, yield: 95%).

The crude was purified by prep-HPLC (Column: Phenomenex luna C18 150×25 mm×10 m; Condition: water (HCl)-ACN; Begin B: 0; End B: 16; Gradient Time (min): 10; 100% B Hold Time (min): 2; Flow Rate (mL/min): 25) to give the product (HCl salt). The product was diluted with 10 mL aqueous NaHCO₃ and extracted with DCM (10 mL×3). The combined organic layers were washed with brine (20 mL), dried over Na₂SO₄, filtered and concentrated under reduced pressure to give 3-(3-(4-(Aminomethyl)phenyl)-3H-imidazo[4,5-b]pyridin-2-yl)pyridin-2-amine (Intermediate 1, 70.0 mg, yield: 95%) as a light-yellow solid. MS: m/z=317.1 [M+H]⁺. ¹H NMR (400 MHz, Dimethylsulfoxide-d₆) δ 8.31 (dd, J=4.8, 1.2 Hz, 1H), 8.19 (dd, J=8.0, 1.2 Hz, 1H), 7.99 (dd, J=4.8, 1.6 Hz, 1H), 7.47 (d, J=8.4 Hz, 2H), 7.40-7.33 (m, 3H), 7.22 (dd, J=7.6, 2.0 Hz, 1H), 6.98 (br s, 2H), 6.40 (dd, J=7.6, 4.8 Hz, 1H), 3.79 (s, 2H), 1.82 (br s, 2H).

### Intermediate 2: 3-(3-(4-(Piperazin-1-ylmethyl)phenyl)-3H-imidazo[4,5-b]pyridin-2-yl)pyridin-2-amine

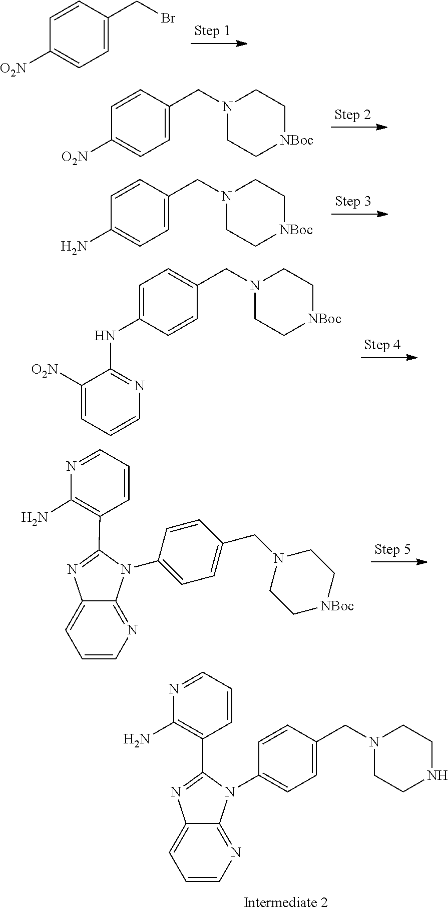

### Step 1: tert-Butyl 4-(4-nitrobenzyl)piperazine-1-carboxylate

To a solution of 1-(bromomethyl)-4-nitrobenzene (25 g, 116 mmol) in ACN (250 mL) were added tert-butyl piperazine-1-carboxylate (25.8 g, 139 mmol) and K₂CO₃ (31.9 g, 231 mmol) at 20° C. The mixture was stirred at 20° C. for 12 hr. The reaction mixture was filtered and concentrated to give tert-butyl 4-(4-nitrobenzyl)piperazine-1-carboxylate (37 g, yield: 99%) as a white solid, which was used in the next step without further purification. MS: m/z=322.2 [M+H]⁺. ¹H NMR (400 MHz, Dimethylsulfoxide-d₆) δ 8.19 (d, J=8.8 Hz, 2H), 7.60 (d, J=8.8 Hz, 2H), 3.62 (s, 2H), 3.32-3.36 (m, 4H), 2.38-2.28 (m, 4H), 1.39 (s, 9H).

### Step 2: tert-Butyl 4-(4-aminobenzyl)piperazine-1-carboxylate

To a solution of tert-butyl 4-(4-nitrobenzyl)piperazine-1-carboxylate (20 g, 62.2 mmol) in EtOH (150 mL) and H₂O (50 mL) were added Fe (17.3 g, 311 mmol) and NH₄Cl (13.3 g, 249 mmol) at 25° C. The mixture was stirred at 90° C. for 2 hr. The reaction mixture was filtered and concentrated to give tert-butyl 4-(4-aminobenzyl)piperazine-1-carboxylate (17 g, crude) as a yellow oil, which was used in the next step without further purification. MS: m/z=292.9 [M+H]⁺.

### Step 3: tert-Butyl 4-(4-((3-nitropyridin-2-yl)amino)benzyl)piperazine-1-carboxylate

To a solution of 2-chloro-3-nitro-pyridine (10 g, 63 mmol) in DMSO (200 mL) were added tert-butyl 4-(4-aminobenzyl)piperazine-1-carboxylate (15.3 g, 52.5 mmol) and DIEA (13.5 g, 105 mmol) at 25° C. The mixture was stirred at 80° C. for 12 hr. The reaction mixture was concentrated, diluted with H₂O (200 mL) and extracted with CH₂Cl₂ (200 mL×2). The combined organic layers were washed with brine (200 mL), dried over Na₂SO₄, filtered, and concentrated under reduced pressure. The residue was purified by silica gel flash chromatography (Eluent of 10~30% EtOAc in petroleum ether) to give tert-butyl 4-(4-((3-nitropyridin-2-yl)amino)benzyl)piperazine-1-carboxylate (11 g, yield: 45%) as a red solid. MS: m/z=413.9 [M+H]⁺. ¹H NMR (400 MHz, Dimethylsulfoxide-d₆) δ 9.95 (s, 1H), 8.48-8.58 (m, 2H), 7.60 (d, J=8.4 Hz, 2H), 7.29 (d, J=8.4 Hz, 2H), 6.98 (dd, J=8.4, 4.8 Hz, 1H), 3.46 (s, 2H), 3.29-3.32 (m, 4H), 2.29-2.35 (m, 4H), 1.39 (s, 9H).

### Step 4: tert-Butyl 4-(4-(2-(2-Aminopyridin-3-yl)-3H-imidazo[4,5-b]pyridin-3-yl)benzyl)piperazine-1-carboxylate

To a solution of tert-butyl 4-(4-((3-nitropyridin-2-yl)amino)benzyl)piperazine-1-carboxylate (10 g, 24.1 mmol) in DMSO (200 mL) were added 2-aminopyridine-3-carbaldehyde (3.54 g, 29.0 mmol) and Na₂S₂O₄ (12.6 g, 72.5 mmol) at 25° C. The mixture was stirred at 100° C. for 14 hr. The reaction mixture was poured into H₂O (500 mL) and extracted with CH₂Cl₂ (200 mL×2). The combined organic layers were washed with brine (400 mL), dried over anhydrous Na₂SO₄, filtered, and concentrated. The residue was purified by silica gel flash chromatography (Eluent of 10~30% MeOH in CH₂Cl₂) to give 4-(4-(2-(2-Aminopyridin-3-yl)-3H-imidazo[4,5-b]pyridin-3-yl)benzyl)piperazine-1-carboxyla (4.9 g, yield: 42%) as a red solid. MS: m/z=486.2 [M+H]⁺. ¹H NMR (400 MHz, Dimethylsulfoxide-d₆) δ 8.33 (dd, J=4.8, 1.2 Hz, 1H), 8.20 (dd, J=8.0, 1.2 Hz, 1H), 7.99 (dd, J=4.8, 2.0 Hz, 1H), 7.43-7.48 (m, 2H), 7.36-7.42 (m, 3H), 7.16 (dd, J=7.6, 1.6 Hz, 1H), 7.00 (br s, 2H), 6.38 (dd, J=7.6, 4.8 Hz, 1H), 3.56 (s, 2H), 3.33-3.37 (m, 4H), 2.32-2.38 (m, 4H), 1.40 (s, 9H).

### Step 5: 3-(3-(4-(Piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-3H-imidazo[4,5-b]pyridin-3-yl)benzyl)piperazine-1-carboxylate (2 g, 4.12 mmol) in CH₂Cl₂ (10 mL) was added dropwise TFA (4.62 g, 40.5 mmol) at 25° C. The mixture was stirred at 25° C. for 3 hr. The reaction mixture was concentrated. The residue was poured into water (50 mL). The pH of the mixture was adjusted to about 8 with saturated NaHCO₃ (aq). The resulting mixture was extracted with CH₂Cl₂ (100 mL×3). The combined organic layers were dried over anhydrous Na₂SO₄, filtered, and concentrated to give 3-(3-(4-(Piperazin-1-ylmethyl)phenyl)-3H-imidazo[4,5-b]pyridin-2-yl)pyridin-2-amine (1.43 g, yield: 90%) as an off-white solid. The solid (100 mg) was triturated with EtOAc (3 mL) at 25° C. for 1 hr and filtered. The filter cake was collected to give 3-(3-(4-(Piperazin-1-ylmethyl)phenyl)-3H-imidazo[4,5-b]pyridin-2-yl)pyridin-2-amine (Intermediate 2, 24.5 mg, yield: 90%). MS: m/z=386.2 [M+H]⁺. ¹H NMR (400 MHz, Dimethylsulfoxide-d₆) δ 8.33 (d, J=4.0 Hz, 1H), 8.22 (d, J=8.0 Hz, 1H), 8.00 (d, J=3.2 Hz, 1H), 7.35-7.52 (m, 5H), 7.18 (d, J=7.2 Hz, 1H), 7.00 (br s, 2H), 6.40 (dd, d, J=7.8, 4.8 Hz, 1H), 3.63 (s, 2H), 3.16-3.01 (m, 4H), 2.63-2.48 (ms, 4H).

### Intermediate 3: 3-(3-(4-((4-Aminopiperidin-1-yl)methyl)phenyl)-3H-imidazo[4,5-b]pyridin-2-yl)pyridin-2-amine

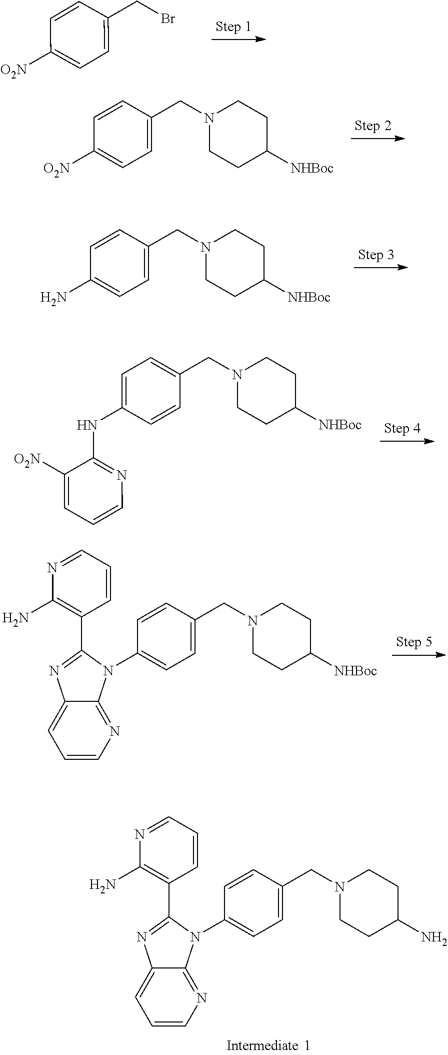

### Step 1: tert-Butyl (1-(4-nitrobenzyl) piperidin-4-yl) carbamate

To a solution of 1-(bromomethyl)-4-nitro-benzene (108 g, 499 mmol) in ACN (1.5 L) were added K₂CO₃ (149 g, 1.1 mol) and tert-butyl N-(4-piperidyl)carbamate (100 g, 499 mmol). The mixture was stirred at 25° C. for 16 hr. The reaction mixture was filtered. The filtrate was concentrated under reduced pressure to give tert-butyl (1-(4-nitrobenzyl) piperidin-4-yl)carbamate (167 g, crude) as a yellow solid, which was used in the next step without further purification. MS: m/z=335.9 [M+H]⁺. ¹H NMR (400 MHz, Chloroform-d) δ 8.16 (d, J=8.8 Hz, 2H), 7.49 (d, J=8.8 Hz, 2H), 4.44 (br s, 1H), 3.56 (s, 2H), 3.52-3.42 (m, 1H), 2.77-2.74 (m, 2H), 2.16-2.10 (m, 2H), 1.93-1.90 (m, 2H), 1.43 (s, 9H), 1.42-1.36 (m, 2H).

### Step 2: tert-Butyl (1-(4-aminobenzyl) piperidin-4-yl) carbamate

To a solution of tert-butyl (1-(4-nitrobenzyl) piperidin-4-yl)carbamate (109 g, 325 mmol) in EtOH (500 mL) and H₂O (150 mL) were added Fe (91 g, 1.6 mol) and NH₄Cl (174 g, 3.3 mol). The mixture was stirred at 85° C. for 2 hr. The reaction mixture was filtered. The filtrate was concentrated under pressure to remove most of the EtOH. The residue was diluted with H₂O (500 mL) and extracted with CH₂Cl₂ (500 mL×2). The combined organic layers were washed with brine (500 mL), dried over Na₂SO₄, filtered, and concentrated under reduced pressure to give tert-butyl (1-(4-aminobenzyl) piperidin-4-yl) carbamate (80 g crude) as a yellow solid. MS: m/z=306.2 [M+H]⁺. ¹H NMR (400 MHz, Chloroform-d) δ 7.14 (d, J=8.0 Hz, 2H), 6.64 (d, J=8.0 Hz, 2H), 4.51 (br d, J=6.0 Hz, 1H), 3.80-3.59 (m, 2H), 3.55 (s, 2H), 3.51-3.39 (m, 1H), 2.95-2.93 (m, 2H), 2.25-2.20 (m, 2H), 1.96-1.93 (m, 2H), 1.72-1.54 (m, 2H), 1.42 (s, 9H).

### Step 3: tert-Butyl (1-(4-((3-nitropyridin-2-yl) amino)benzyl)piperidin-4-yl)carbamate

To a solution of tert-butyl (1-(4-aminobenzyl) piperidin-4-yl) carbamate (30 g, 98.2 mmol) in DMSO (500 mL) were added DIEA (38.1 g, 295 mmol) and 2-chloro-3-nitro-pyridine (18.7 g, 118 mmol). The mixture was stirred at 100° C. for 16 hr. The reaction mixture was quenched with H₂O (500 mL) at 20° C. and extracted with EtOAc (300 mL×2). The combined organic layers were washed with brine (300 mL), dried over Na₂SO₄, filtered, and concentrated under reduced pressure. The crude was purified by silica gel flash chromatography (Eluent of 0~10% MeOH in CH₂Cl₂) to give tert-butyl (1-(4-((3-nitropyridin-2-yl) amino)benzyl) piperidin-4-yl) carbamate (30 g, yield: 71%) as a yellow solid. MS: m/z=428.2 [M+H]⁺. ¹H NMR (400 MHz, Chloroform-d) δ 10.10 (s, 1H), 8.51 (dd, J=8.0, 1.6 Hz, 1H), 8.47 (dd, J=8.4, 1.6 Hz, 1H), 7.58 (d, J=8.4 Hz, 2H), 7.32 (d, J=8.4 Hz, 2H), 6.81 (dd, J=8.4, 4.4 Hz, 1H), 4.44 (br s, 1H), 3.47 (s, 2H), 3.44-3.34 (m, 1H), 2.82-2.79 (m, 2H), 2.13-2.05 (m, 2H), 1.93-1.89 (m, 2H), 1.43 (s, 9H), 1.39-1.37 (m, 2H).

### Step 4: tert-Butyl (1-(4-(2-(2-aminopyridin-3-yl)-3H-imidazo[4,5-b]pyridin-3-yl)benzyl)piperidin-4-yl)carbamate

To a solution of (12.5 g, 29.2 mmol) in DMSO (500 mL) were added Na₂S₂O₄ (15.3 g, 87.7 mmol) and 2-aminopyridine-3-carbaldehyde (4.3 g, 35.1 mmol). The mixture was stirred at 100° C. for 16 hr. The reaction mixture was quenched with H₂O (1000 mL) at 20° C. and extracted with EtOAc (1000 mL×2). The combined organic layers were washed with brine (500 mL), dried over Na₂SO₄, filtered, and concentrated under reduced pressure. The crude was purified by silica gel flash chromatography (Eluent of 0-10% MeOH in CH₂Cl₂) to give tert-butyl (1-(4-(2-(2-aminopyridin-3-yl)-3H-imidazo[4,5-b]pyridin-3-yl)benzyl)piperidin-4-yl)carbamate (5.5 g, yield: 38%) as a yellow solid. MS: m/z=500.2 [M+H]⁺. ¹H NMR (400 MHz, Chloroform-d) δ 8.46-8.37 (m, 1H), 8.12-8.02 (m, 2H), 7.48 (d, J=8.0 Hz, 2H), 7.34-7.28 (m, 3H), 7.07 (dd, J=8.0, 4.0 Hz, 1H), 6.62 (br s, 2H), 6.33 (dd, J=7.6, 4.8 Hz, 1H), 4.46 (br d, J=6.0 Hz, 1H), 3.56 (s, 2H), 3.49-3.47 (m, 1H), 2.84 (br d, J=11.2 Hz, 2H), 2.14 (t, J=12.0 Hz, 2H), 1.93 (br d, J=11.2 Hz, 2H), 1.45 (s, 9H), 1.51-1.38 (m, 2H).

### Step 5: 3-(3-(4-((4-Aminopiperidin-1-yl)methyl)phenyl)-3H-imidazo[4,5-b]pyridin-2-yl)pyridin-2-amine

A solution of tert-butyl (1-(4-(2-(2-aminopyridin-3-yl)-3H-imidazo[4,5-b]pyridin-3-yl)benzyl)piperidin-4-yl)carbamate (2.0 g, 4.0 mmol) in HCl/1,4-dioxane (4M, 20 mL) was stirred at 25° C. for 1 hr. The reaction mixture was concentrated under reduced pressure to remove solvent. The residue was quenched with aq. NaHCO₃ (30 mL) at 20° C. MeOH was added, filtered. The filtrate was freeze-dried to give 3-(3-(4-((4-aminopiperidin-1-yl)methyl)phenyl)-3H-imidazo[4,5-b]pyridin-2-yl)pyridin-2-amine (Intermediate 3, 1.45 g, yield: 91%) as a yellow solid. MS: m/z=400.1 [M+H]⁺. ¹H NMR (400 MHz, Dimethylsulfoxide-d₆) δ 8.37-8.28 (m, 1H), 8.19 (dd, J=8.0, 4.0 Hz, 1H), 7.99-7.97 (m, 1H), 7.47-7.32 (m, 5H), 7.15 (dd, J=7.6, 1.6 Hz, 1H), 7.01 (br s, 2H), 6.36 (dd, J=6.8, 4.0 Hz, 1H), 3.50 (s, 2H), 3.27-3.23 (m, 1H), 2.75 (br d, J=10.8 Hz, 2H), 2.00 (t, J=10.8 Hz, 2H), 1.72 (d, J=10.8 Hz, 2H), 1.39-1.31 (m, 2H).

### Intermediate 4: 3-(3-(4-(Aminomethyl)phenyl)-5-phenyl-3H-imidazo[4,5-b]pyridin-2-yl)pyridin-2-amine

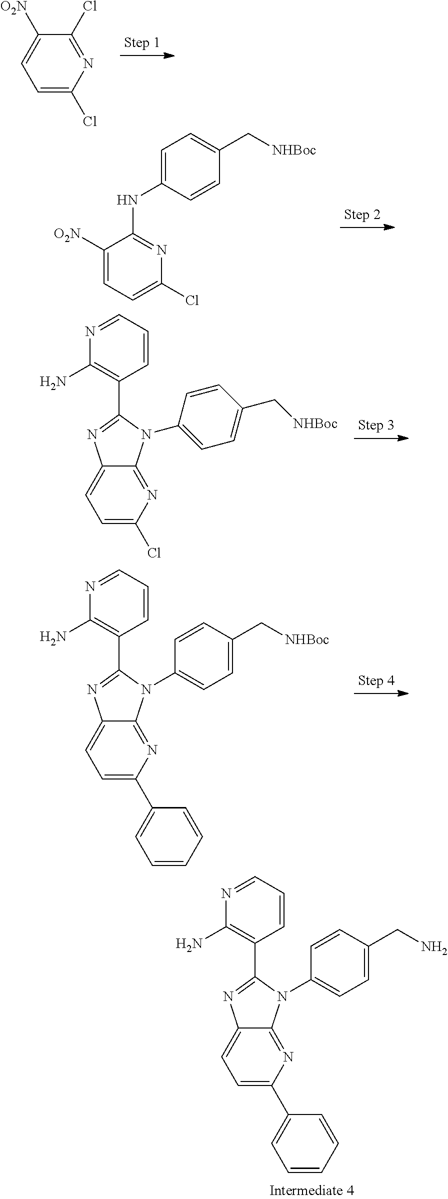

### Step 1: tert-Butyl 4-((6-chloro-3-nitropyridin-2-yl)amino)benzylcarbamate

To a solution of 2,6-dichloro-3-nitro-pyridine (2.0 g, 10.4 mmol) and tert-butyl N-[(4-aminophenyl)methyl]carbamate (2.3 g, 10.4 mmol) in DMSO (25 mL) was added DIEA (4.0 g, 31.1 mmol). The mixture was stirred at 80° C. for 12 hr. The reaction mixture was quenched with H₂O (50 mL) at 25° C. and extracted with EtOAc (50 mL×3). The combined organic layers were washed with brine (50 mL×2), dried over Na₂SO₄, filtered, and concentrated under reduced pressure. The crude was purified by silica gel flash chromatography (Eluent of 0~10% MeOH in CH₂Cl₂) to give tert-butyl N-[[4-[(6-chloro-3-nitro-2-pyridyl)amino]phenyl]methyl]carbamate (2.4 g, yield: 44%) as a red solid. MS: m/z=400.9 [M+Na]⁺. ¹H NMR (400 MHz, Dimethylsulfoxide-d₆) δ 10.10 (s, 1H), 8.53 (d, J=8.8 Hz, 1H), 7.52 (d, J=8.4 Hz, 2H), 7.40 (t, J=6.0 Hz, 1H), 7.25 (d, J=8.4 Hz, 2H), 6.99 (d, J=8.8 Hz, 1H), 4.13 (d, J=6.0 Hz, 2H), 1.40 (s, 9H).

### Step 2: tert-Butyl N-[[4-[2-(2-amino-3-pyridyl)-5-chloro-imidazo[4,5-b]pyridin-3-yl]phenyl]methyl]carbamate

To a solution of tert-butyl N-[[4-[(6-chloro-3-nitro-2-pyridyl)amino]phenyl]methyl]carbamate (2.0 g, 5.3 mmol) in DMSO (30 mL) and MeOH (15 mL) were added 2-aminopyridine-3-carbaldehyde (0.7 g, 5.8 mmol) and Na₂S₂O₄ (1.8 g, 10.6 mmol). The mixture was stirred at 100° C. for 16 hr. The reaction mixture was quenched with H₂O (50 mL) at 25° C. and extracted with EtOAc (100 mL×3). The combined organic layers were washed with brine (100 mL×2), dried over Na₂SO₄, filtered, and concentrated under reduced pressure. The crude was purified by silica gel flash chromatography (Eluent of 0~10% MeOH in CH₂Cl₂) to give tert-butyl N-[[4-[2-(2-amino-3-pyridyl)-5-chloro-imidazo[4,5-b]pyridin-3-yl]phenyl]methyl]carbamate (1.4 g, yield: 51%) as a yellow solid. MS: m/z=451.0 [M+H]⁺. ¹H NMR (400 MHz, Dimethylsulfoxide-d₆) δ 8.26 (d, J=8.4 Hz, 1H), 7.99 (dd, J=4.8, 1.6 Hz, 1H), 7.50-7.43 (m, 2H), 7.38 (s, 4H), 7.21 (dd, J=7.6, 1.6 Hz, 1H), 6.91 (br s, 2H), 6.40 (dd, J=7.6, 4.8 Hz, 1H), 4.22 (d, J=6.0 Hz, 2H), 1.41 (s, 9H).

### Step 3: tert-Butyl N-[[4-[2-(2-amino-3-pyridyl)-5-phenyl-imidazo[4,5-b]pyridin-3-yl]phenyl]methyl]carbamate

To a solution of tert-butyl N-[[4-[2-(2-amino-3-pyridyl)-5-chloro-imidazo[4,5-b]pyridin-3-yl]phenyl]methyl]carbamate (500 mg, 1.1 mmol) and phenylboronic acid (270 mg, 2.2 mmol,) in 1,4-dioxane (5 mL) and H₂O (1 mL) were added Pd(dppf)Cl₂ (81.1 mg, 111 mol) and Cs₂CO₃ (1.1 g, 3.3 mmol). The mixture was degassed and purged with N₂ three times and stirred at 80° C. for 16 hr under N₂ atmosphere. After cooling to 20° C., the reaction was diluted with EtOAc (10 mL), filtered through celite and extracted with H₂O (10 mL×3). The combined organic layers were washed with brine (15 mL×3), dried over Na₂SO₄, filtered and concentrated under reduced pressure. The crude was purified by silica gel flash chromatography (Eluent of 10~35% EtOAc in petroleum ether) to give tert-butyl N-[[4-[2-(2-amino-3-pyridyl)-5-phenyl-imidazo[4,5-b]pyridin-3-yl]phenyl]methyl]carbamate (126 mg, yield: 21%) as a brown solid. MS: m/z=493.2 [M+H]⁺.

### Step 4: 3-(3-(4-(Aminomethyl)phenyl)-5-phenyl-3H-imidazo[4,5-b]pyridin-2-yl)pyridin-2-amine

A solution of tert-butyl N-[[4-[2-(2-amino-3-pyridyl)-5-phenyl-imidazo[4,5-b]pyridin-3-yl]phenyl]methyl]carbamate (126 mg, 256 μmol) in HCl/1,4-dioxane (4 M, 1 mL) was stirred at 25° C. for 2 hr. The solvent was removed under reduced pressure to give a crude product (84 mg, yield: 84%). The crude was purified by prep-HPLC (column: Welch Xtimate C18 150×25 mm×5 μm; mobile phase: [water (HCl)-ACN]; B %: 5%-35%, 8 min) to give the desired product (HCl salt). The product was diluted with aqueous NaHCO₃ (10 mL) and extracted with DCM (10 mL×3). The combined organic layers were washed with brine (10 mL), dried over Na₂SO₄, filtered, and concentrated under reduced pressure to give 3-(3-(4-(aminomethyl)phenyl)-5-phenyl-3H-imidazo[4,5-b]pyridin-2-yl)pyridin-2-amine (Intermediate 4, 32.2 mg, yield: 84%) as a light-yellow solid. MS: m/z=393.1 [M+H]⁺. ¹H NMR (400 MHz, Dimethylsulfoxide-d₆) δ 8.26 (d, J=8.4 Hz, 1H), 8.04-7.96 (m, 4H), 7.54-7.49 (m, 2H), 7.49-7.43 (m, 2H), 7.43-7.35 (m, 3H), 7.21 (dd, J=7.6, 1.6 Hz, 1H), 6.98 (br s, 2H), 6.41 (dd, J=7.6, 4.8 Hz, 1H), 3.82 (s, 2H).

### Intermediate 5: N-(3-(3-(4-(Aminomethyl)phenyl)-2-(2-aminopyridin-3-yl)-3H-imidazo[4,5-b]pyridin-5-yl)phenyl)acetamide

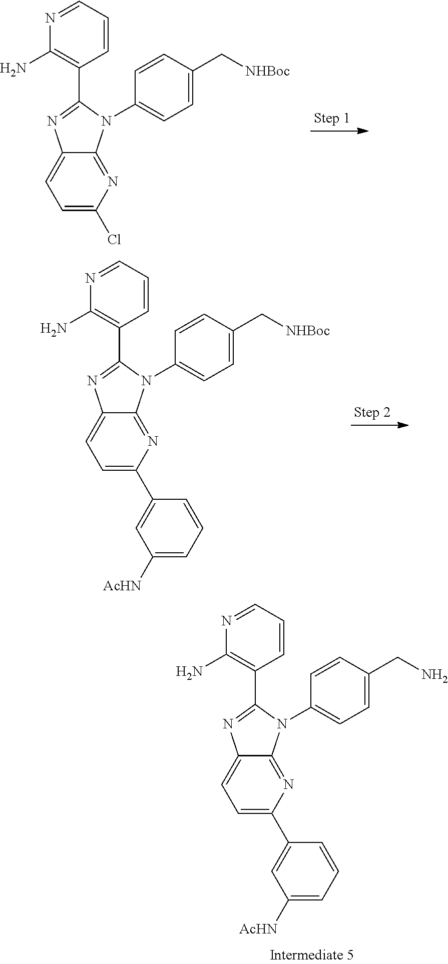

### Step 1: tert-Butyl 4-(5-(3-acetamidophenyl)-2-(2-aminopyridin-3-yl)-3H-imidazo[4,5-b]pyridin-3-yl)benzylcarbamate

To a solution of tert-butyl N-[[4-[2-(2-amino-3-pyridyl)-5-chloro-imidazo[4,5-b]pyridin-3-yl]phenyl]methyl]carbamate (refer to Intermediate 4 for detail procedures, 3.5 g, 7.8 mmol) and (3-acetamidophenyl)boronic acid (2.8 g, 15.5 mmol) in 1,4-dioxane (30 mL) and H₂O (6 mL) were added Pd(dppf)Cl₂ (568 mg, 776 μmol) and Cs₂CO₃ (7.6 g, 23.3 mmol). The mixture was was degassed and purged with N₂ three times and then stirred at 80° C. for 16 hr under N₂ atmosphere. The reaction mixture was quenched with H₂O (50 mL) at 25° C. and extracted with EtOAc (50 mL×3). The combined organic layers were washed with brine (50 mL×2), dried over Na₂SO₄, filtered, and concentrated under reduced pressure to give tert-butyl N-[[4-[5-(3-acetamidophenyl)-2-(2-amino-3-pyridyl)imidazo[4,5-b]pyridin-3-yl]phenyl]methyl]carbamate (2.43 g, yield: 57%), which was used in the next step without further purification. MS: m/z=550.1 [M+H]⁺.

### Step 2: N-(3-(3-(4-(Aminomethyl)phenyl)-2-(2-aminopyridin-3-yl)-3H-imidazo[4,5-b]pyridin-5-yl)phenyl)acetamide

A solution of tert-butyl N-[[4-[5-(3-acetamidophenyl)-2-(2-amino-3-pyridyl)imidazo[4,5-b]pyridin-3-yl]phenyl]methyl]carbamate (8.0 g, 14.6 mmol) in HCl/1,4-dioxane (4 M, 20 mL) was stirred at 25° C. for 2 hr. The solvent was removed under reduced pressure to give a crude product (6.4 g, HCl salt, yield: 90%). The crude (250 mg, HCl salt) was diluted with aqueous NaHCO₃ (10 mL) and extracted with CH₂Cl₂ (10 mL×3). The combined organic layers were washed with brine (10 mL), dried over Na₂SO₄, filtered and concentrated under reduced pressure to give N-(3-(3-(4-(aminomethyl)phenyl)-2-(2-aminopyridin-3-yl)-3H-imidazo[4,5-b]pyridin-5-yl)phenyl)acetamide (Intermediate 5, 86.7 mg, yield: 90%) as a light-yellow solid. MS: m/z=450.0 [M+H]⁺. ¹H NMR (400 MHz, Dimethylsulfoxide-d₆) δ 10.08 (s, 1H), 8.26 (d, J=8.4 Hz, 1H), 8.10 (s, 1H), 8.00 (dd, J=4.8, 1.6 Hz, 1H), 7.84 (d, J=8.4 Hz, 1H), 7.68 (d, J=8.0 Hz, 1H), 7.65 (d, J=8.0 Hz, 1H), 7.53-7.48 (m, 2H), 7.45-7.35 (m, 3H), 7.20 (dd, J=7.6, 1.6 Hz, 1H), 6.94 (br s, 2H), 6.41 (dd, J=7.6, 4.8 Hz, 1H), 3.82 (s, 2H), 2.06 (s, 3H).

### Intermediate 6: N-(3-(2-(2-Aminopyridin-3-yl)-3-(4-(piperazin-1-ylmethyl)phenyl)-3H-imidazo[4,5-b]pyridin-5-yl)phenyl)acetamide

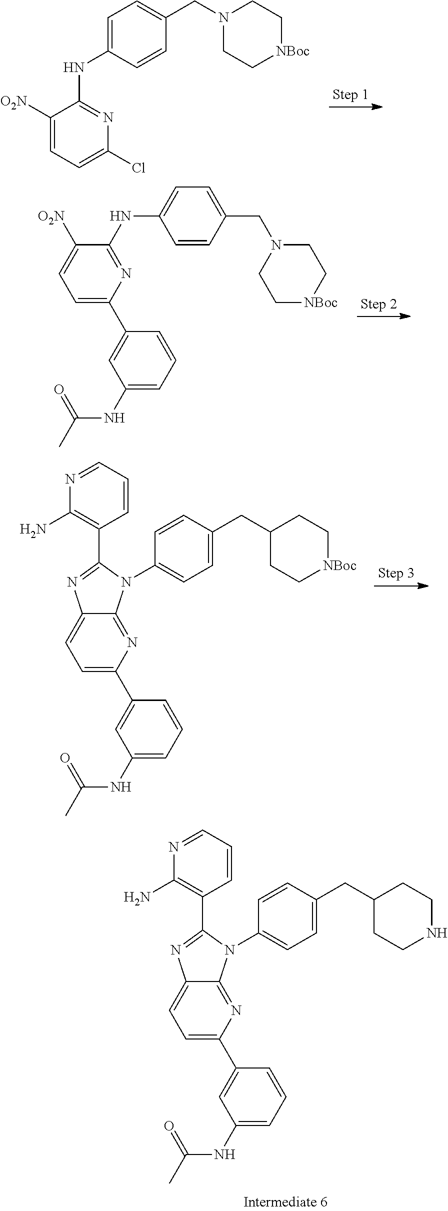

### Step 1: tert-Butyl 4-(4-((6-(3-Acetamidophenyl)-3-nitropyridin-2-yl)amino)benzyl)piperazine-1-carboxylate

To a solution of tert-butyl 4-[[4-[(6-chloro-3-nitro-2-pyridyl)amino]phenyl]methyl]piperazine-1-carboxylate (2.78 g, 5.39 mmol) and (3-acetamidophenyl)boronic acid (1.93 g, 10.78 mmol) in 1,4-dioxane (30 mL) and H₂O (6 mL) were added K₂CO₃ (2.24 g, 16.2 mmol) and Pd(dppf)Cl₂ (789 mg, 1.08 mmol). The mixture was degassed and purged with N₂ three times and stirred at 60° C. for 16 hr under N₂ atmosphere. The reaction mixture was quenched H₂O (200 mL) at 25° C. and extracted with CH₂Cl₂ (60 mL×3). The combined organic layers were washed with brine (100 mL×2), dried over Na₂SO₄, filtered, and concentrated under reduced pressure. The residue was purified by silica gel flash chromatography (Eluent of 50~100% EtOAc in petroleum ether) to give tert-Butyl 4-(4-((6-(3-Acetamidophenyl)-3-nitropyridin-2-yl)amino)benzyl)piperazine-1-carboxylate (2.81 g, yield: 83%) as a red solid. MS: m/z=547.1 [M+H]⁺. ¹H NMR (400 MHz, Chloroform-d) δ 10.19 (s, 1H), 8.42 (d, J=8.8 Hz, 1H), 8.19 (br s, 1H), 7.68-7.60 (m, 4H), 7.47 (d, J=9.2 Hz, 1H), 7.34-7.27 (m, 2H), 7.17-7.12 (m, 1H), 3.43 (s, 2H), 3.35 (br t, J=4.8 Hz, 4H), 2.32 (br t, J=4.8 Hz, 4H), 2.11 (s, 3H), 1.36 (s, 9H).

### Step 2: tert-Butyl 4-(4-(5-(3-acetamidophenyl)-2-(2-aminopyridin-3-yl)-3H-imidazo[4,5-b]pyridin-3-yl)benzyl)piperazine-1-carboxylate

To a solution of 2-aminopyridine-3-carbaldehyde (2.3 g, 18.9 mmol) and tert-Butyl 4-(4-((6-(3-Acetamidophenyl)-3-nitropyridin-2-yl)amino)benzyl)piperazine-1-carboxylate (9.5 g, 17.2 mmol) in DMSO (100 mL) was added Na₂S₂O₄ (8.97 g, 51.5 mmol). The mixture was degassed and purged with N₂ three times and stirred at 60° C. for 16 hr under N₂ atmosphere. The reaction mixture was quenched with H₂O (400 mL) at 25° C. and extracted with CH₂Cl₂ (400 mL×2). The combined organic layers were washed with brine (300 mL×2), dried over Na₂SO₄, filtered, and concentrated under reduced pressure to give tert-Butyl 4-(4-(5-(3-acetamidophenyl)-2-(2-aminopyridin-3-yl)-3H-imidazo[4,5-b]pyridin-3-yl)benzyl)piperazine-1-carboxylate (9.67 g, crude) as a red solid, which was used in the next step without further purification. MS: m/z=619.2 [M+H]⁺.

### Step 3: N-(3-(2-(2-Aminopyridin-3-yl)-3-(4-(piperazin-1-ylmethyl)phenyl)-3H-imidazo[4,5-b]pyridin-5-yl)phenyl)acetamide

A solution of tert-Butyl 4-(4-(5-(3-acetamidophenyl)-2-(2-aminopyridin-3-yl)-3H-imidazo[4,5-b]pyridin-3-yl)benzyl)piperazine-1-carboxylate (0.4 g, 370 μmol, crude) in HCl/1,4-dioxane (4M, 5 mL) and 1,4-dioxane (1 mL) was degassed and purged with N₂ three times and stirred at 25° C. for 4 hr under N₂ atmosphere. The reaction mixture was filtered. The filter cake was washed with 1,4-dioxane (10 mL×2) and the filtrate was concentrated under reduced pressure. The residue was purified by prep-HPLC (column: Phenomenex Luna C18 200*40 mm*10 μm; mobile phase: [water (HCl)-ACN]; B %: 1%-30%, 10 min) to give N-(3-(2-(2-aminopyridin-3-yl)-3-(4-(piperazin-1-ylmethyl)phenyl)-3H-imidazo[4,5-b]pyridin-5-yl)phenyl)acetamide (Intermediate 6, 244 mg HCl salt, yield: 68%) as a yellow solid. MS: m/z=519.2 [M+H]⁺. ¹H NMR (400 MHz, Dimethylsulfoxide-d₆) δ 10.05 (s, 1H), 8.27 (d, J=8.4 Hz, 1H), 8.15 (s, 1H), 7.99 (dd, J=4.8, 1.6 Hz, 1H), 7.86 (d, J=8.4 Hz, 1H), 7.66 (d, J=8.0 Hz, 2H), 7.50-7.33 (m, 5H), 7.14 (dd, J=7.6, 1.2 Hz, 1H), 7.00 (br s, 2H), 6.37 (dd, J=7.6, 4.8 Hz, 1H), 3.52 (s, 2H), 2.77-2.67 (m, 4H), 2.41-2.27 (m, 4H), 2.05 (s, 3H).

### Intermediate 7: 3-[3-[4-(aminomethyl)phenyl]-6-phenyl-imidazo[4,5-b]pyridin-2-yl]pyridin-2-amine

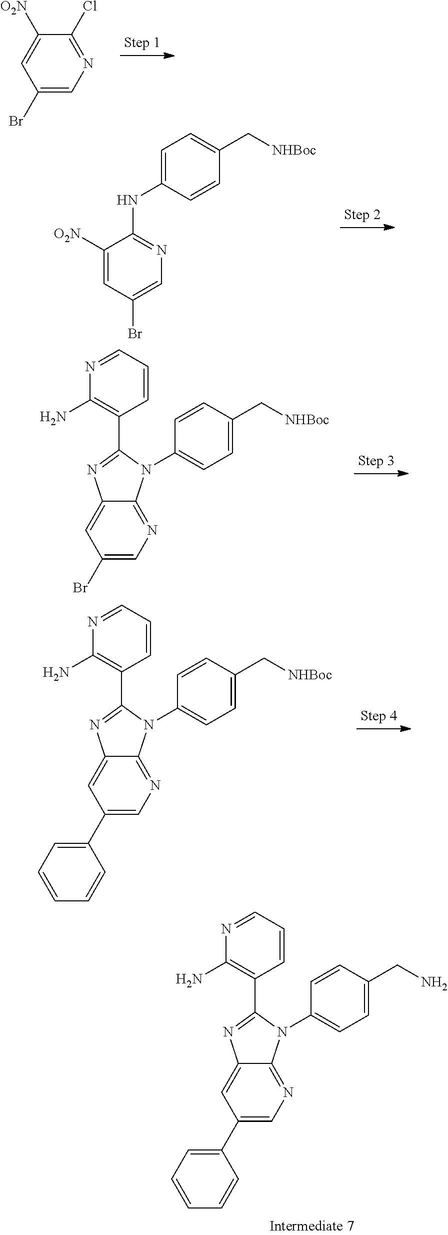

### Step 1: tert-Butyl N-[[4-[(5-bromo-3-nitro-2-pyridyl)amino]phenyl]methyl]carbamate

To a solution of 5-bromo-2-chloro-3-nitro-pyridine (2.1 g, 9.0 mmol) and tert-butyl N-[(4-aminophenyl)methyl]carbamate (2 g, 9.0 mmol) in DMSO (20 mL) was added DIEA (3.5 g, 27.0 mmol). The mixture was stirred at 80° C. for 12 hr. After cooling to 20° C., the mixture was diluted with H₂O (50 mL) and extracted with EtOAc (50 mL×2). The combined organic layers were washed with brine (50 mL×2), dried over Na₂SO₄, filtered, and concentrated under reduced pressure to give tert-butyl N-[[4-[(5-bromo-3-nitro-2-pyridyl)amino]phenyl]methyl]carbamate (3.5 g crude, yield: 92%) as a red solid. MS: m/z=367.6, 368.6 [M+H]⁺. ¹H NMR (400 MHz, Chloroform-d)) δ 10.02 (s, 1H), 8.65 (d, J=2.0 Hz, 1H), 8.49 (d, J=2.0 Hz, 1H), 7.55 (d, J=8.4 Hz, 2H), 7.32 (d, J=8.0 Hz, 2H), 4.84 (br s, 1H), 4.32 (d, J=5.2 Hz, 2H), 1.47 (s, 9H).

### Step 2: tert-Butyl N-[[4-[2-(2-amino-3-pyridyl)-6-bromo-imidazo[4,5-b]pyridin-3-yl]phenyl]methyl]carbamate

To a solution of tert-butyl N-[[4-[(5-bromo-3-nitro-2-pyridyl)amino]phenyl]methyl]carbamate (3 g, 7.1 mmol) and 2-aminopyridine-3-carbaldehyde (952 mg, 7.8 mmol) in DMSO (30 mL) and MeOH (15 mL) was added Na₂S₂O₄ (2.5 g, 14.2 mmol). The mixture was stirred at 100° C. for 12 hr. After cooling to 20° C., the reaction mixture was diluted with H₂O (50 mL) and extracted with EtOAc (50 mL×2). The combined organic layers were washed with brine (50 mL×2), dried over Na₂SO₄, filtered and concentrated under reduced pressure. The residue was purified by silica gel flash chromatography (Eluent of 0~1% MeOH in CH₂Cl₂) to give tert-butyl-N-[[4-[2-(2-amino-3-pyridyl)-6-bromo-imidazo[4,5-b]pyridin-3-yl]phenyl]methyl]carbamate (1.7 g, yield: 36%) as a yellow solid. MS: m/z=495.9, 496.9 [M+H]⁺. ¹H NMR (400 MHz, Chloroform-d)) δ 8.40 (d, J=2.0 Hz, 1H), 8.22 (d, J=2.0 Hz, 1H), 8.08 (dd, J=4.8, 2.0 Hz, 1H), 7.46 (d, J=8.4 Hz, 2H), 7.33 (d, J=8.4 Hz, 2H), 7.10 (dd, J=8.0, 2.0 Hz, 1H), 6.63 (br s, 2H), 6.41-6.34 (m, 1H), 4.93 (br s, 1H), 4.42 (d, J=5.6 Hz, 2H), 1.48 (s, 9H).

### Step 3: tert-Butyl N-[[4-[2-(2-amino-3-pyridyl)-6-phenyl-imidazo[4,5-b]pyridin-3-yl]phenyl]methyl]carbamate

To a solution of tert-butyl N-[[4-[2-(2-amino-3-pyridyl)-6-bromo-imidazo[4,5-b]pyridin-3-yl]phenyl]methyl]carbamate (500 mg, 1.0 mmol) and phenylboronic acid (246 mg, 2.0 mmol) in toluene (5 mL) and EtOH (5 mL) were added NaHCO₃ (254 mg, 3.0 mmol) and Pd(PPh₃)₄(233 mg, 202 mol). The mixture was degassed and purged with N₂ three times and stirred at 100° C. for 12 hr under N₂ atmosphere. After cooling to 20° C., the reaction mixture was diluted with H₂O (20 mL) and extracted with EtOAc (20 mL×2). The combined organic layers were washed with saturated NaHCO₃ solution (20 mL×2), dried over Na₂SO₄, filtered, and concentrated under reduced pressure. The residue was purified by silica gel flash chromatography (Eluent of 0-1% MeOH in CH₂Cl₂) to give tert-butyl N-[[4-[2-(2-amino-3-pyridyl)-6-phenyl-imidazo[4,5-b]pyridin-3-yl]phenyl]methyl]carbamate (340 mg, 62% yield) as a brown solid. MS: m/z=493.1 [M+H]⁺. 1H NMR (400 MHz, Dimethylsulfoxide-d₆)) δ 8.61 (d, J=2.0 Hz, 1H), 8.45 (d, J=2.0 Hz, 1H), 8.01-7.99 (m, 1H), 7.77 (d, J=7.2 Hz, 2H), 7.63-7.59 (m, 4H), 7.54-7.52 (m, 2H), 7.39 (d, J=5.2 Hz, 2H), 7.23 (dd, J=7.6, 2.0 Hz, 1H), 7.04 (br s, 2H), 6.40 (dd, J=7.6, 5.2 Hz, 1H), 4.21 (d, J=6.0 Hz, 2H), 1.40 (s, 9H).

### Step 4: 3-[3-[4-(Aminomethyl)phenyl]-6-phenyl-imidazo[4,5-b]pyridin-2-yl]pyridin-2-amine

To a solution of tert-butyl N-[[4-[2-(2-amino-3-pyridyl)-6-phenyl-imidazo[4,5-b]pyridin-3-yl]phenyl]methyl]carbamate (340 mg, 690 μmol, 1.0 eq) in 1,4-dioxane (5 mL) was added HCl/dioxane (5 mL). The mixture was stirred at 25° C. for 1 hr. The reaction mixture was concentrated under reduced pressure to give the crude (297 mg, HCl salt, yield: 96%). The residue was purified by prep-HPLC (column: Welch Xtimate C₁₈ 150×25 mm×5 μm; mobile phase: [water (HCl)-ACN]; B %: 3%-33%, 8 min) and dissociated with NaHCO₃ to give 3-[3-[4-(aminomethyl)phenyl]-6-phenyl-imidazo[4,5-b]pyridin-2-yl]pyridin-2-amine (Intermediate 7, 83.8 mg, yield: 96%) as an off-white solid. MS: m/z=393.1 [M+H]⁺. ¹H NMR (400 MHz, Dimethylsulfoxide-d₆)) δ 8.62 (d, J=2.0 Hz, 1H), 8.46 (d, J=2.0 Hz, 1H), 8.00 (dd, J=4.8, 2.0 Hz, 1H), 7.78 (d, J=7.2 Hz, 2H), 7.56-7.46 (m, 5H), 7.45-7.35 (m, 4H), 7.26 (dd, J=8.0, 2.0 Hz, 1H), 7.04 (s, 2H), 6.44-6.39 (m, 1H), 3.80 (s, 2H).

### Intermediate 8: 3-(6-Phenyl-3-(4-(piperazin-1-ylmethyl)phenyl)-3H-imidazo[4,5-b]pyridin-2-yl)pyridin-2-amine

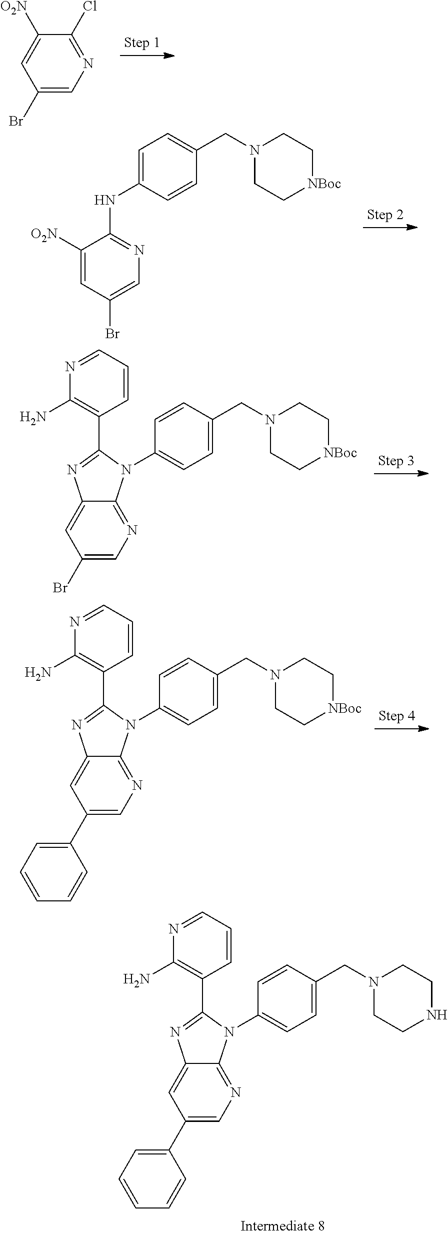

### Step 1: tert-Butyl 4-(4-((5-bromo-3-nitropyridin-2-yl)amino)benzyl)piperazine-1-carboxylate

To a solution of 5-bromo-2-chloro-3-nitro-pyridine (10 g, 42.1 mmol) in DMSO (100 mL) were added tert-butyl 4-[(4-aminophenyl)methyl]piperazine-1-carboxylate (11 g, 30.2 mmol) and DIEA (11.7 g, 94.8 mmol). The mixture was stirred at 80° C. for 12 hr. After cooling to 20° C., the reaction mixture was poured into H₂O (100 mL). The aqueous layer was extracted with EtOAc (200 mL×3). The combined organic layers were washed with brine (200 mL×3). The combined organic layers were dried over anhydrous Na₂SO₄, filtered, and concentrated to dryness. The crude was purified by silica gel flash chromatography (Elute of 10~50% EtOAc in petroleum ether) to give tert-butyl 4-[[4-[(5-bromo-3-nitro-2-pyridyl)amino]phenyl]methyl]piperazine-1-carboxylate (10.7 g, yield: 43%) as a brown solid. MS: m/z=493.8 [M+H]⁺. ¹H NMR (400 MHz, Dimethylsulfoxide-d₆) δ 9.93 (s, 1H), 8.67 (d, J=2.4 Hz, 1H), 8.60 (d, J=2.4 Hz, 1H), 8.50 (dd, J=10.0, 2.4 Hz, 1H), 7.54 (d, J=8.4 Hz, 2H), 7.29 (d, J=8.0 Hz, 1H), 3.46 (s, 2H), 3.32-3.28 (m, 4H), 2.32-2.30 (m, 4H), 1.39 (s, 9H).

### Step 2: tert-Butyl 4-(4-(2-(2-aminopyridin-3-yl)-6-bromo-3H-imidazo[4,5-b]pyridin-3-yl)benzyl)piperazine-1-carboxylate

To a solution of tert-butyl 4-[[4-[(5-bromo-3-nitro-2-pyridyl)amino]phenyl]methyl]piperazine-1-carboxylate (10.7 g, 21.6 mmol) in DMSO (60 mL) and methanol (30 mL) were added 2-aminopyridine-3-carbaldehyde (3.2 g, 26 mmol) and Na₂S₂O₄ (7.5 g, 43.3 mmol). The reaction mixture was stirred at 100° C. for 16 hr. After cooling to 25° C., the reaction mixture was diluted with H₂O (100 mL) and extracted with CH₂Cl₂ (100 mL×3) and brine (100 mL×2 times). The combined organic phases were dried over Na₂SO₄, filtered, and concentrated under reduced pressure. tert-Butyl 4-[[4-[2-(2-amino-3-pyridyl)-6-bromo-imidazo[4,5-b]pyridin-3-yl]phenyl]methyl]piperazine-1-carboxylate (7.6 g, yield: 62%) was obtained as a red oil. MS: m/z=565.9 [M+H]⁺.

### Step 3: tert-Butyl 4-(4-(2-(2-aminopyridin-3-yl)-6-phenyl-3H-imidazo[4,5-b]pyridin-3-yl)benzyl)piperazine-1-carboxylate

To a solution of tert-butyl 4-[[4-[2-(2-amino-3-pyridyl)-6-bromo-imidazo[4,5-b]pyridin-3-yl]phenyl]methyl]piperazine-1-carboxylate (5.8 g, 10.3 mmol) and phenylboronic acid (2.5 g, 20.6 mmol) in 1,4-dioxane (100 mL) and H₂O (10 mL) were added Pd(dppf)Cl₂ (752 mg, 1.03 mmol) and Cs₂CO₃ (10 g, 30.8 mmol). The mixture was degassed and purged with N₂ three times and stirred at 80° C. for 16 hr under N₂ atmosphere. After cooling to 25° C., the reaction mixture was poured into H₂O (100 mL) and extracted with EtOAc (100 mL×3). The combined organic layers were washed with brine (100 mL×3). The combined organic layers were dried over anhydrous Na₂SO₄, filtered and concentrated under reduced pressure to give tert-butyl 4-[[4-[2-(2-amino-3-pyridyl)-6-phenyl-imidazo[4,5-b]pyridin-3-yl]phenyl]methyl]piperazine-1-carboxylate (2.54 g, yield: 44%) as a brown oil. MS: m/z=562.4 [M+H]⁺.

### Step 4: 3-(6-Phenyl-3-(4-(piperazin-1-ylmethyl)phenyl)-3H-imidazo[4,5-b]pyridin-2-yl)pyridin-2-amine

A solution of tert-butyl 4-[[4-[2-(2-amino-3-pyridyl)-6-phenyl-imidazo[4,5-b]pyridin-3-yl]phenyl]methyl]piperazine-1-carboxylate (10 g, 17.80 mmol, 1 eq) in HCl/1,4-dioxane (4M, 100 mL) was stirred at 25° C. for 1 hr. The reaction mixture was filtered and concentrated under reduced pressure to give a crude product. The crude was triturated with MeOH (30 mL) at 25° C. for 30 min. The suspension was filtered. The filter cake was washed with MeOH (20 mL) and dried under reduced pressure to give 3-(6-phenyl-3-(4-(piperazin-1-ylmethyl)phenyl)-3H-imidazo[4,5-b]pyridin-2-yl)pyridin-2-amine (Intermediate 8, 3.7 g, yield: 42%) as a yellow solid. MS: m/z=462.2 [M+H]⁺. ¹H NMR (400 MHz, Dimethylsulfoxide-d₆) δ 8.63 (d, J=2.0 Hz, 1H), 8.46 (d, J=2.0 Hz, 1H), 8.00 (dd, J=4.8, 1.6 Hz, 1H), 7.81-7.75 (m, 2H), 7.52 (dd, J=7.6, 7.6 Hz, 2H), 7.47-7.38 (m, 5H), 7.19 (dd, J=7.6, 1.6 Hz, 1H), 7.06 (br s, 2H), 6.39 (dd, J=7.6, 4.8 Hz, 1H), 3.51 (s, 2H), 3.33-3.27 (m, 1H), 2.71 (br t, J=4.4 Hz, 4H), 2.32 (s, 4H).

### Intermediate 9: 3-[3-[4-[(4-Amino-1-piperidyl)methyl]phenyl]-6-phenyl-imidazo[4,5-b]pyridin-2-yl]pyridin-2-amine

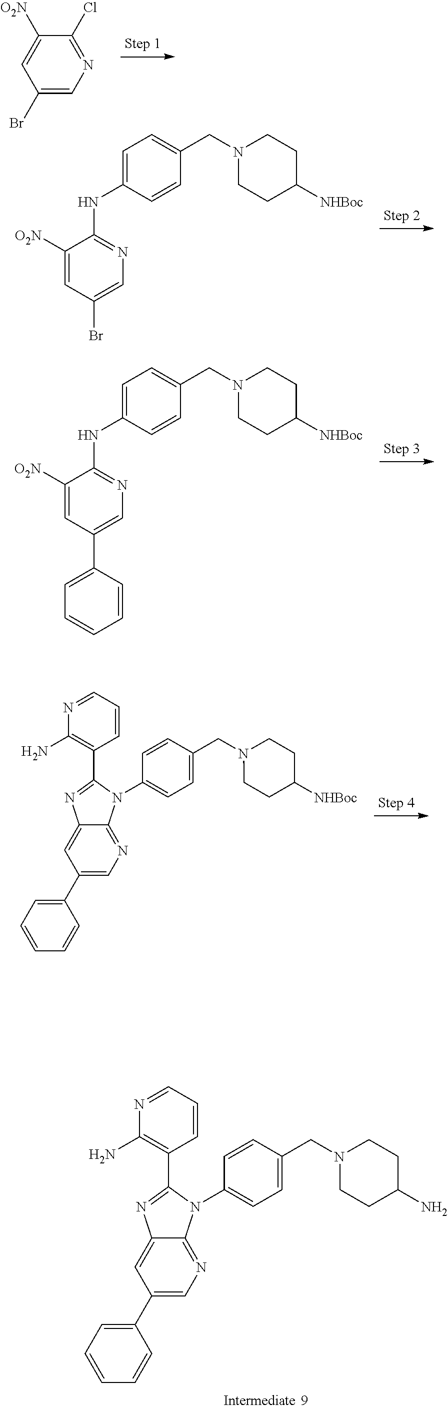

### Step 1: tert-Butyl N-[1-[[4-[(5-bromo-3-nitro-2-pyridyl)amino]phenyl]methyl]-4-piperidyl]carbamate

A mixture of 5-bromo-2-chloro-3-nitro-pyridine (17.1 g, 72 mmol), tert-butyl N-[1-[(4-aminophenyl)methyl]-4-piperidyl]carbamate (22 g, 72 mmol), DIEA (27.9 g, 216 mmol) in DMSO (200 mL) was stirred at 80° C. for 16 hr. After cooling to 25° C., the mixture was extracted with EtOAc (250 mL×3). The combined organic layers were washed with brine (200 mL×2), dried over anhydrous Na₂SO₄, filtered, and concentrated to give tert-butyl N-[1-[[4-[(5-bromo-3-nitro-2-pyridyl)amino]phenyl]methyl]-4-piperidyl]carbamate 32 g crude product as black brown solid. MS: m/z=506.9, 507.9 [M+H]⁺. ¹H NMR (400 MHz, Chloroform-d) δ 10.04 (s, 1H), 8.64 (d, J=2.0 Hz, 1H), 8.49 (d, J=2.4 Hz, 1H), 7.53 (d, J=8.4 Hz, 2H), 7.33 (d, J=8.4 Hz, 2H), 4.34 (d, J=3.2 Hz, 1H), 3.48 (s, 2H), 3.46-3.35 (m, 1H), 2.82 (br d, J=12.0 Hz, 2H), 2.10 (t, J=10.8 Hz, 2H), 1.91 (br d, J=11.2 Hz, 2H), 1.44 (s, 9H).

### Step 2: tert-Butyl N-[1-[[4-[(3-nitro-5-phenyl-2-pyridyl)amino]phenyl]methyl]-4-piperidyl]carbamate

A mixture of tert-butyl N-[1-[[4-[(5-bromo-3-nitro-2-pyridyl)amino]phenyl]methyl]-4-piperidyl]carbamate (20 g, 39.5 mmol), phenylboronic acid (4.8 g, 39.5 mmol), K₂CO₃ (16.4 g, 118.5 mmol), Pd(dppf)Cl₂ (1.4 g, 2.0 mmol) in 1,4-dioxane (250 mL) and H₂O (50 mL) was degassed and purged with N₂ three times. The mixture was stirred at 80° C. for 16 hr under N₂ atmosphere. After cooling to 25° C., the reaction mixture was filtered, diluted with H₂O (100 mL) and EtOAc (450 mL). The organic phase was separated, washed with brine (100 mL×3), dried over Na₂SO₄, filtered, and concentrated under reduced pressure. The residue was purified by silica gel flash chromatography (Eluent of 3~4% MeOH in CH₂Cl₂) to give tert-butyl-[1-[[4-[(3-nitro-5-phenyl-2-pyridyl)amino]phenyl]methyl]-4-piperidyl]carbamate (8.6 g, yield: 43%) as a red brown solid. MS: m/z=504.2 [M+H]⁺. ¹H NMR (400 MHz, Chloroform-d) δ 10.14 (s, 1H), 8.75 (dd, J=10.0, 2.0 Hz, 2H), 7.62 (d, J=8.0, Hz, 2H), 7.57 (d, J=7.2, Hz, 2H), 7.49 (dd, J=8.0, 8.0 Hz, 2H), 7.43-7.37 (m, 3H), 4.43 (br s, 1H), 3.50 (br s, 2H), 3.49-3.41 (m, 1H), 2.85-2.81 (m, 2H), 2.15-2.08 (m, 2H), 1.94-1.91 (m, 2H), 1.51-1.45 (m, 2H), 1.44 (s, 9H).

### Step 3. tert-Butyl N-[1-[[4-[2-(2-amino-3-pyridyl)-6-phenyl-imidazo[4,5-b]pyridin-3-yl]phenyl]methyl]-4-piperidyl]carbamate

A mixture of tert-butyl N-[1-[[4-[(3-nitro-5-phenyl-2-pyridyl)amino]phenyl]methyl]-4-piperidyl]carbamate (2.2 g, 4.4 mmol), 2-aminopyridine-3-carbaldehyde (694 mg, 5.7 mmol), Na₂S₂O₄ (1.5 g, 8.7 mmol) in DMSO (100 mL) was degassed and purged with N₂ three times, The mixture was stirred at 100° C. for 16 hr under N₂ atmosphere. After cooling to 25° C., the reaction mixture was filtered, diluted with H₂O (30 mL) and extracted with EtOAc (45 mL). The organic phase was separated, washed with brine (10 mL×3), dried over Na₂SO₄, filtered and concentrated under reduced pressure. The residue was purified by silica gel flash chromatography (Eluent of 5~6% EtOAc in petroleum ether) to give tert-butyl N-[1-[[4-[2-(2-amino-3-pyridyl)-6-phenyl-imidazo[4,5-b]pyridin-3-yl]phenyl]methyl]-4-piperidyl]carbamate (700 mg, yield: 28%) as a black brown solid. MS: m/z=576.2 [M+H]⁺. ¹H NMR (400 MHz, Chloroform-d) δ 8.63 (d, J=2.0 Hz, 1H), 8.27 (d, J=2.0 Hz, 1H), 8.07 (dd, J=5.2, 2.0 Hz, 1H), 7.67-7.62 (m, 2H), 7.54-7.47 (m, 4H), 7.44-7.39 (m, 1H), 7.36 (d, J=8.4 Hz, 2H), 7.09 (dd, J=8.0, 2.0 Hz, 1H), 6.65 (br s, 2H), 6.35 (dd, J=8.0, 4.8 Hz, 1H), 4.45 (br s, 1H), 3.58 (s, 2H), 3.55-3.46 (m, 1H), 2.92-2.77 (m, 2H), 2.16 (br t, J=10 Hz, 2H), 1.95 (br d, J=11.2 Hz, 2H), 1.63-1.56 (m, 2H), 1.45 (s, 9H).

### Step 4: 3-[3-[4-[(4-Amino-1-piperidyl)methyl]phenyl]-6-phenyl-imidazo[4,5-b]pyridin-2-yl]pyridin-2-amine

A mixture of tert-butyl N-[1-[[4-[2-(2-amino-3-pyridyl)-6-phenyl-imidazo[4,5-b]pyridin-3-yl]phenyl]methyl]-4-piperidyl]carbamate (2.4 g, 4.17 mmol) in HCl/1,4-dioxane (4M, 20 mL) and MeOH (4 mL) was stirred at 25° C. for 2 hr. The reaction mixture was filtered to give a residue (2 g HCl salt, yield: 94.3%). The crude (100 mg) was purified by prep-HPLC (column: Welch Ultimate C18 150×25 mm×5 μm; mobile phase: [water (FA)-ACN]; B %: 0% to 25%, 10 min) to give 3-[3-[4-[(4-amino-1-piperidyl)methyl]phenyl]-6-phenyl-imidazo[4,5-b]pyridin-2-yl]pyridin-2-amine (Intermediate 9, 26.6 mg, 2HCOOH salt, yield: 94%) as a yellow solid. MS: m/z=476.1 [M+H]⁺. ¹H NMR (400 MHz, Dimethylsulfoxide-d₆) δ 8.63 (d, J=2.0 Hz, 1H), 8.47 (d, J=2.0 Hz, 1H), 8.34 (s, 2H), 8.00 (dd, J=4.8, 1.6 Hz, 1H), 7.78 (d, J=7.6 Hz, 2H), 7.53 (dd, J=7.6, 7.6 Hz, 2H), 7.47-7.39 (m, 5H), 7.20 (dd, J=7.6, 1.6 Hz, 1H), 7.04 (br s, 2H), 6.39 (dd, J=7.6, 4.8 Hz, 1H), 3.55 (s, 2H), 2.98-2.88 (m, 1H), 2.87-2.80 (m, 2H), 2.09-1.97 (m, 2H), 1.90-1.80 (m, 2H), 1.57-1.44 (m, 2H).

### Intermediate 10: 3-[5-Phenyl-3-[4-(piperazin-1-ylmethyl)phenyl]imidazo[4,5-b]pyridin-2-yl]pyridin-2-amine

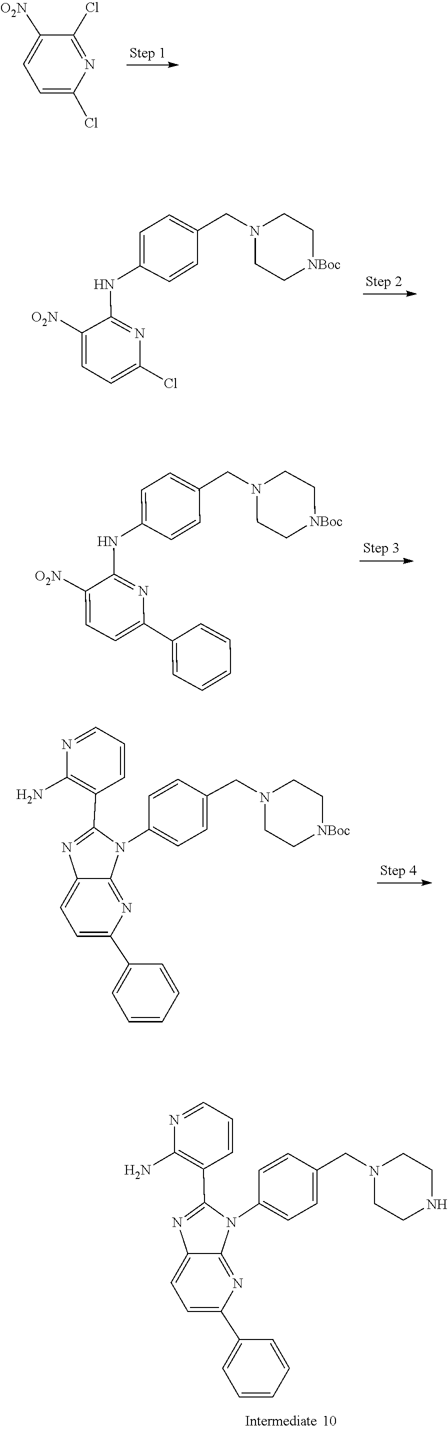

### Step 1: tert-Butyl 4-[[4-[(6-chloro-3-nitro-2-pyridyl)amino]phenyl]methyl]piperazine-1-carboxylate

To a solution of 2,6-dichloro-3-nitro-pyridine (5.0 g, 25.9 mmol) in 1,4-dioxane (50 mL) were added DIEA (6.7 g, 51.8 mmol) and tert-butyl 4-[(4-aminophenyl)methyl]piperazine-1-carboxylate (10.8 g, 25.9 mmol). The mixture was stirred at 60° C. for 12 hr. The reaction mixture was diluted with H₂O (50 mL) and extracted with EtOAc (100 mL×3). The combined organic layers were washed with brine (50 mL×2), dried over Na₂SO₄, filtered, and concentrated under reduced pressure. The residue was purified by silica gel flash chromatography (Eluent of 0-50% EtOAc in petroleum ether) to give tert-butyl 4-[[4-[(6-chloro-3-nitro-2-pyridyl)amino]phenyl]methyl]piperazine-1-carboxylate (6.1 g, yield: 53%) as a yellow solid. MS: m/z=447.9 [M+H]⁺. ¹H NMR (400 MHz, Chloroform-d) δ 10.27 (s, 1H), 8.46 (d, J=8.8 Hz, 1H), 7.61 (d, J=8.4 Hz, 2H), 7.36 (d, J=8.4 Hz, 2H), 6.80 (d, J=8.4 Hz, 1H), 3.52 (s, 2H), 3.48-3.37 (m, 4H), 2.45-2.32 (m, 4H), 1.46 (s, 9H).

### Step 2. tert-Butyl 4-[[4-[(3-nitro-6-phenyl-2-pyridyl)amino]phenyl]methyl]piperazine-1-carboxylate

To a solution of tert-butyl 4-[[4-[(6-chloro-3-nitro-2-pyridyl)amino]phenyl]methyl]piperazine-1-carboxylate (1.0 g, 2.23 mmol) and phenylboronic acid (544 mg, 4.47 mmol) in 1,4-dioxane (10 mL) and H₂O (2 mL) were added Pd(dppf)Cl₂ (327 mg, 0.446 mmol) and K₂CO₃ (926 mg, 6.7 mmol). The mixture was stirred at 60° C. for 4 hr. The reaction mixture was added with H₂O (50 mL) and extracted with EtOAc (100 mL×3). The combined organic layers were washed with brine (50 mL×2), dried over Na₂SO₄, filtered, and concentrated under reduced pressure to give a residue. The residue was purified by silica gel flash chromatography (Eluent of 0-50% EtOAc in petroleum ether) to give tert-butyl 4-[[4-[(3-nitro-6-phenyl-2-pyridyl)amino]phenyl]methyl]piperazine-1-carboxylate (1.0 g, yield: 92%) as a red solid. MS: m/z=490.1 [M+H]⁺. ¹H NMR (400 MHz, Chloroform-d) δ 10.31 (s, 1H), 8.59 (d, J=8.8 Hz, 1H), 8.06-8.04 (m, 2H), 7.73 (d, J=8.4 Hz, 2H), 7.52-7.44 (m, 3H), 7.37 (d, J=8.4 Hz, 2H), 7.3 (d, J=8.4 Hz, 1H), 3.54 (s, 2H), 3.52-3.37 (m, 4H), 2.49-2.37 (m, 4H), 1.46 (s, 9H).

### Step 3: tert-Butyl 4-[[4-[2-(2-amino-3-pyridyl)-5-phenyl-imidazo[4,5-b]pyridin-3-yl]phenyl]methyl]piperazine-1-carboxylate

To a solution of 2-aminopyridine-3-carbaldehyde (269 mg, 2.21 mmol) and tert-butyl 4-[[4-[(3-nitro-6-phenyl-2-pyridyl)amino]phenyl]methyl]piperazine-1-carboxylate (900 mg, 1.84 mmol) in DMSO (10 mL) was added Na₂S₂O₄ (960 mg, 5.52 mmol) at 15° C. The mixture was stirred at 100° C. for 20 hr. The reaction mixture was diluted with H₂O (50 mL) and extracted with DCM (80 mL×3). The combined organic layers were washed with brine (80 mL×3), dried over Na₂SO₄, filtered, and concentrated under reduced pressure. The residue was purified by silica gel flash chromatography (Eluent of 0-10% MeOH in CH₂Cl₂) to give tert-butyl 4-[[4-[2-(2-amino-3-pyridyl)-5-phenyl-imidazo[4,5-b]pyridin-3-yl]phenyl]methyl]piperazine-1-carboxylate (600 mg, yield: 58%) as a yellow solid. MS: m/z=562.1 [M+H]⁺. ¹H NMR (400 MHz, Chloroform-d) δ 8.13 (d, J=8.4 Hz, 1H), 8.06 (dd, J=5.2, 2.0 Hz, 1H), 8.03-8.01 (m, 2H), 7.81 (d, J=8.4 Hz, 1H), 7.51-7.49 (m, 2H), 7.48-7.35 (m, 5H), 7.10 (dd, J=9.6, 2.0 Hz, 1H), 6.66 (br s, 2H), 6.36 (dd, J=8.0, 4.0 Hz, 1H), 3.64 (s, 2H), 3.53-3.42 (m, 4H), 2.55-2.42 (m, 4H), 1.47 (s, 9H).

### Step 4: 3-[5-Phenyl-3-[4-(piperazin-1-ylmethyl)phenyl]imidazo[4,5-b]pyridin-2-yl]pyridin-2-amine

A solution of tert-butyl 4-[[4-[2-(2-amino-3-pyridyl)-5-phenyl-imidazo[4,5-b]pyridin-3-yl]phenyl]methyl]piperazine-1-carboxylate (400 mg, 712 μmol) in HCl/1,4-dioxane (4M, 8 mL) was stirred at 25° C. for 4 hr. The reaction was filtered and concentrated under reduced pressure to give 3-[5-phenyl-3-[4-(piperazin-1-ylmethyl)phenyl]imidazo[4,5-b]pyridin-2-yl]pyridin-2-amine (450 mg, HCl) as a yellow solid, which was used in the next step without further purification. The crude (200 mg) was purified by prep-HPLC (column: Waters xbridge 150*25 mm 10 μm; mobile phase: [water (NH₄HCO₃)-ACN]; B %: 19%-49%, 9 min) to give 3-[5-Phenyl-3-[4-(piperazin-1-ylmethyl)phenyl]imidazo[4,5-b]pyridin-2-yl]pyridin-2-amine (Intermediate 10, 49.7 mg) as a yellow solid. MS: m/z=462.2 [M+H]⁺. ¹H NMR (400 MHz, Dimethylsulfoxide-d₆) δ 8.27 (d, J=8.4 Hz, 1H), 8.04-7.97 (m, 4H), 7.48-7.39 (m, 8H), 7.15 (dd, J=8.0, 2.0 Hz, 1H), 7.03 (br s, 2H), 6.38 (dd, J=7.6, 4.8 Hz, 1H), 3.53 (s, 2H), 2.74-2.66 (m, 4H), 2.38-2.27 (m, 4H).

### Intermediate 11: 3-(3-(4-((4-Aminopiperidin-1-yl)methyl)phenyl)-5-phenyl-3H-imidazo[4,5-b]pyridin-2-yl)pyridin-2-amine

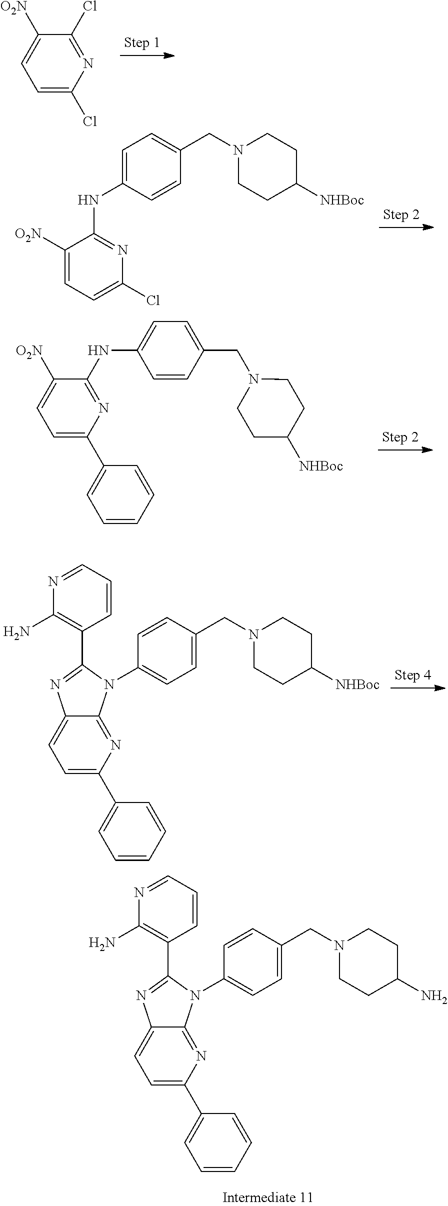

### Step 1: tert-Butyl (1-(4-((6-chloro-3-nitropyridin-2-yl)amino)benzyl)piperidin-4-yl)carbamate

To a solution of 2,6-dichloro-3-nitro-pyridine (3.0 g, 15.5 mmol) and tert-butyl N-[1-[(4-aminophenyl)methyl]-4-piperidyl]carbamate (4.8 g, 15.6 mmol) in 1,4-dioxane (100 mL) was added DTEA (6.0 g, 46.6 mmol). The mixture was stirred at 50° C. for 12 hr. The reaction mixture was diluted with H₂O (50 mL) and extracted with EtOAc (30 mL×2). The combined organic layers were washed with brine (50 mL), dried over Na₂SO₄, filtered, and concentrated under reduced pressure. The residue was purified by silica gel flash chromatography (Eluent of 0~5% MeOH in CH₂Cl₂) to give tert-butyl N-[1-[[4-[(6-chloro-3-nitro-2-pyridyl)amino]phenyl]methyl]-4-piperidyl]carbamate (4.6 g, yield: 64%) as an orange solid. MS: m/z=462.1 [M+H]⁺. ¹H NMR (400 MHz, Chloroform-d) δ 10.26 (s, 1H), 8.46 (d, J=8.8 Hz, 1H), 7.60 (d, J=8.8 Hz, 2H), 7.34 (d, J=8.4 Hz, 2H), 6.79 (d, J=8.4 Hz, 1H), 4.42 (br s, 1H), 3.48 (s, 2H), 3.48-3.39 (m, 1H), 2.82 (br d, J=10.8 Hz, 2H), 2.10 (br t, J=10.8 Hz, 2H), 1.92 (br d, J=10.8 Hz, 2H), 1.50-1.40 (m, 2H). 1.44 (s, 9H).

### Step 2: tert-Butyl (1-(4-((3-nitro-6-phenylpyridin-2-yl)amino)benzyl)piperidin-4-yl)carbamate

A mixture of tert-butyl N-[1-[[4-[(6-chloro-3-nitro-2-pyridyl)amino]phenyl]methyl]-4-piperidyl]carbamate (1.0 g, 2.20 mmol), phenylboronic acid (528 mg, 4.30 mmol), Pd(dppf)Cl₂ (158 mg, 0.216 mmol) and K₂CO₃ (898 mg, 6.50 mmol) in 1,4-dioxane (10 mL) and H₂O (2 mL) was degassed and purged with N₂ three times. The mixture was stirred at 100° C. for 12 hr under N₂ atmosphere. After cooling to 25° C., the reaction mixture was diluted with H₂O and extracted with DCM (50 mL×2). The combined organic layers were washed with brine (50 mL), dried over Na₂SO₄, filtered, and concentrated under reduced pressure. The residue was purified by silica gel flash chromatography (Eluent of 0-7% MeOH in CH₂Cl₂) to give tert-butyl N-[1-[[4-[(3-nitro-6-phenyl-2-pyridyl)amino]phenyl]methyl]-4-piperidyl]carbamate (1.1 g, yield: 96%) as a yellow solid. MS: m/z=504.1 [M+H]⁺. ¹H NMR (400 MHz, Chloroform-d) δ 10.31 (s, 1H), 8.59 (d, J=8.8 Hz, 1H), 8.09-8.02 (m, 2H), 7.72 (d, J=8.4 Hz, 2H), 7.51-7.47 (m, 3H), 7.36 (d, J=8.8 Hz, 1H), 7.30 (d, J=8.8 Hz, 1H), 4.52-4.37 (m, 1H), 3.52 (s, 2H), 3.51-3.42 (m, 1H), 2.85 (br d, J=11.2 Hz, 2H), 2.12 (br t, J=10.8 Hz, 2H), 1.93 (br d, J=11.0 Hz, 2H), 1.50-1.40 (m, 2H). 1.44 (s, 9H).

### Step 3: tert-Butyl (1-(4-(2-(2-Aminopyridin-3-yl)-5-phenyl-3H-imidazo[4,5-b]pyridin-3-yl)benzyl)piperidin-4-yl) carbamate

A solution of tert-butyl N-[1-[[4-[(3-nitro-6-phenyl-2-pyridyl)amino]phenyl]methyl]-4-piperidyl]carbamate (200 mg, 0.397 mmol), 2-aminopyridine-3-carbaldehyde (53.4 mg, 0.437 mmol) and Na₂S₂O₄ (207 mg, 1.2 mmol) in DMSO (6 mL) was stirred at 100° C. for 18 hr. After cooling to 25° C., the reaction mixture was diluted with DCM (40 mL). The organic layers were washed with H₂O (20 mL) and brine (20 mL), dried over Na₂SO₄, filtered and concentrated under reduced pressure. The residue was purified by silica gel flash chromatography (Eluent of 0~7% MeOH in CH₂Cl₂) to give tert-butyl N-[1-[[4-[2-(2-amino-3-pyridyl)-5-phenyl-imidazo[4,5-b]pyridin-3-yl]phenyl]methyl]-4-piperidyl]carbamate (100 mg, yield: 40%) as a yellow solid. MS: m/z=576.2 [M+H]⁺.

### Step 4: 3-(3-(4-((4-Aminopiperidin-1-yl)methyl)phenyl)-5-phenyl-3H-imidazo[4,5-b]pyridin-2-yl)pyridin-2-amine

A solution of tert-butyl (1-(4-(2-(2-aminopyridin-3-yl)-5-phenyl-3H-imidazo[4,5-b]pyridin-3-yl)benzyl)piperidin-4-yl)carbamate (200 mg, 0.347 mmol) in HCl in 1,4-dioxane (4 M, 2 mL) was stirred at 25° C. for 2 hr. The reaction mixture was concentrated under reduced pressure. The residue was purified by prep-HPLC (column: Waters xbridge 150*25 mm 10 μm; mobile phase: [water (NH₄HCO₃)-ACN]; B %: 24%-54%, 8 min) to give 3-(3-(4-((4-aminopiperidin-1-yl)methyl)phenyl)-5-phenyl-3H-imidazo[4,5-b]pyridin-2-yl)pyridin-2-amine (Intermediate 11, 120 mg, yield: 72%) as a light-yellow solid. MS: m/z=476.2 [M+H]⁺. ¹H NMR (400 MHz, Chloroform-d) δ 8.12 (d, J=8.4 Hz, 1H), 8.05 (dd, J=5.2, 1.6 Hz, 1H), 8.01 (d, J=7.2 Hz, 2H), 7.79 (d, J=8.4 Hz, 1H), 7.50-7.35 (m, 7H), 7.09 (dd, J=7.6, 1.2 Hz, 1H), 6.61 (br s, 2H), 6.35 (dd, J=7.6, 4.8 Hz, 1H), 3.58 (s, 2H), 2.88 (br d, J=11.6 Hz, 2H), 2.75-2.65 (m, 1H), 2.09 (br t, J=11.6 Hz, 2H), 1.83 (br d, J=11.6 Hz, 2H), 1.49-1.38 (m, 2H).

### Intermediate 12: N-(3-(3-(4-((4-Aminopiperidin-1-yl)methyl)phenyl)-2-(2-aminopyridin-3-yl)-3H-imidazo[4,5-b]pyridin-5-yl)phenyl)acetamide

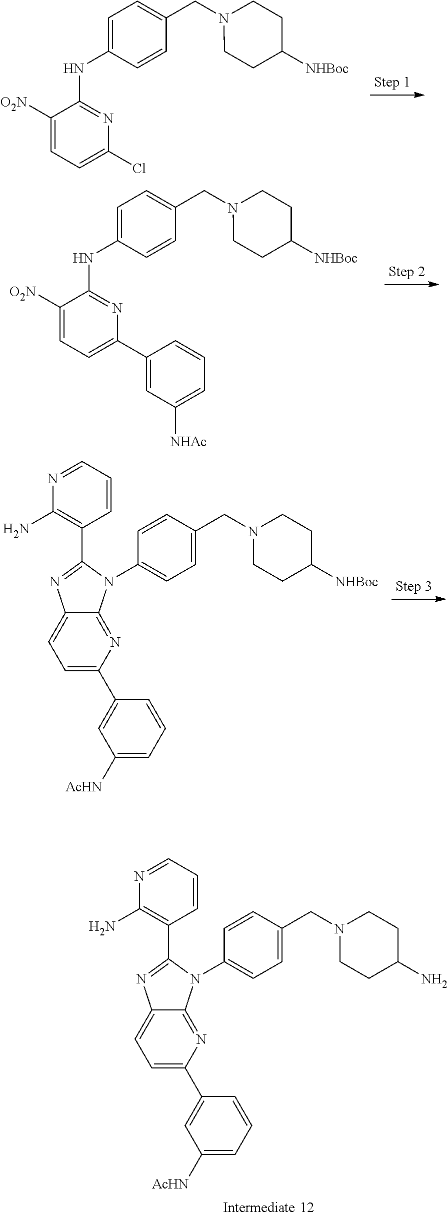

### Step 1: tert-Butyl (1-(4-((6-(3-acetamidophenyl)-3-nitropyridin-2-yl)amino)benzyl)piperidin-4-yl)carbamate

A mixture of tert-butyl N-[1-[[4-[(6-chloro-3-nitro-2-pyridyl)amino]phenyl]methyl]-4-piperidyl]carbamate (1.0 g, 2.16 mmol), (3-acetamidophenyl)boronic acid (773 mg, 4.32 mmol), Pd(dppf)Cl₂ (158 mg, 0.216 mmol) and K₂CO₃ (895 mg, 6.48 mmol) in H₂O (2 mL) and 1,4-dioxane (10 mL) was degassed and purged with N₂ three times. The mixture was stirred at 100° C. for 12 hr under N₂ atmosphere. The reaction mixture was diluted with H₂O (50 mL) and extracted with CH₂Cl₂ (50 mL×2). The combined organic layers were washed with brine (50 mL), dried over Na₂SO₄, filtered, and concentrated under reduced pressure. The residue was purified by silica gel flash chromatography (Eluent of 0-5% MeOH in CH₂Cl₂) to give tert-butyl N-[1-[[4-[[6-(3-acetamidophenyl)-3-nitro-2-pyridyl]amino]phenyl]methyl]-4-piperidyl]carbamate (1.04 g, yield: 86%) as an orange solid. MS: m/z=561.1 [M+H]⁺. ¹H NMR (400 MHz, Chloroform-d) δ 10.30 (s, 1H), 8.57 (d, J=8.8 Hz, 1H), 8.26 (br s, 1H), 7.79 (d, J=8.0 Hz, 1H), 7.72 (d, J=8.4 Hz, 2H), 7.63-7.57 (m, 1H), 7.47-7.35 (m, 4H), 7.28 (d, J=8.8 Hz, 1H), 4.44 (br s, 1H), 3.51 (s, 2H), 3.44-3.48 (m, 1H), 2.93-2.78 (m, 2H), 2.23 (s, 3H), 2.13 (br t, J=10.4 Hz, 2H), 1.93 (br d, J=11.2 Hz, 2H), 1.52-1.45 (m, 2H), 1.44 (s, 9H).

### Step 2: tert-Butyl (1-(4-((3-nitro-6-phenylpyridin-2-yl)amino)benzyl)piperidin-4-yl)carbamate

A solution of tert-butyl N-[1-[[4-[[6-(3-acetamidophenyl)-3-nitro-2-pyridyl]amino]phenyl]methyl]-4-piperidyl]carbamate (950 mg, 1.69 mmol), 2-aminopyridine-3-carbaldehyde (228 mg, 1.9 mmol) and Na₂SO₄ (590 mg, 3.4 mmol) in DMSO (12 mL) was stirred at 100° C. for 18 hr. After cooling to 25° C., the reaction mixture was diluted with CH₂Cl₂ (50 mL), washed with H₂O (50 mL) and brine (50 mL), dried over Na₂SO₄, filtered, and concentrated under reduced pressure. The residue was purified by silica gel flash chromatography (Eluent of 0~7% MeOH in CH₂Cl₂) to give tert-butyl N-[1-[[4-[5-(3-acetamidophenyl)-2-(2-amino-3-pyridyl)imidazo[4,5-b]pyridin-3-yl]phenyl]methyl]-4-piperidyl]carbamate (536 mg, yield: 50%) as a yellow solid. MS: m/z=633.3 [M+H]⁺. ¹H NMR (400 MHz, Chloroform-d) δ 8.11 (d, J=8.4 Hz, 1H), 8.06 (dd, J=4.8, 2.0 Hz, 1H), 8.00 (br s, 1H), 7.78 (d, J=8.4 Hz, 1H), 7.73 (d, J=8.0 Hz, 1H), 7.66 (d, J=8.0 Hz, 1H), 7.49 (br s, 1H), 7.45 (d, J=8.4 Hz, 2H), 7.40-7.34 (m, 3H), 7.05 (dd, J=8.0, 1.6 Hz, 1H), 6.60 (br s, 2H), 6.33 (dd, J=7.6, 4.8 Hz, 1H), 4.47 (br s, 1H), 3.58 (s, 2H), 3.51-3.48 (m, 1H), 2.85 (br d, J=11.2 Hz, 2H), 2.18-2.16 (m, 2H), 2.14 (s, 3H), 1.94 (br d, J=10.8 Hz, 2H), 1.49-1.47 (m, 2H), 1.45 (s, 9H).

### Step 3: N-(3-(3-(4-((4-aminopiperidin-1-yl)methyl)phenyl)-2-(2-aminopyridin-3-yl)-3H-imidazo[4,5-b]pyridin-5-yl)phenyl)acetamide

A solution of tert-butyl N-[1-[[4-[5-(3-acetamidophenyl)-2-(2-amino-3-pyridyl)imidazo[4,5-b]pyridin-3-yl]phenyl]methyl]-4-piperidyl]carbamate (300 mg, 474 μmol) in HCl/1,4-dioxane (4 M, 2 mL) was stirred at 25° C. for 2 hr. The reaction mixture was added NaHCO₃ to adjust the pH to about 8, and extracted with DCM (15 mL×3). The combined organic layers were washed with brine (10 mL×2), dried by Na₂SO₄, filtered and concentrated under reduced pressure to give N-(3-(3-(4-((4-aminopiperidin-1-yl)methyl)phenyl)-2-(2-aminopyridin-3-yl)-3H-imidazo[4,5-b]pyridin-5-yl)phenyl)acetamide (Intermediate 12, 134 mg, yield: 53%) as a yellow solid. MS: m/z=533.2 [M+H]⁺. ¹H NMR (400 MHz, Chloroform-d) δ 8.13 (d, J=8.4 Hz, 1H), 8.07 (dd, J=4.8, 1.6 Hz, 1H), 8.02 (s, 1H), 7.81 (d, J=8.4 Hz, 1H), 7.76 (d, J=8.0 Hz, 1H), 7.69 (d, J=8.0 Hz, 1H), 7.48 (d, J=8.4 Hz, 2H), 7.42-7.37 (m, 4H), 7.08 (d, J=6.8 Hz, 1H), 6.61 (br s, 2H), 6.36 (dd, J=7.6, 4.8 Hz, 1H), 3.59 (s, 2H), 2.90 (br d, J=11.2 Hz, 2H), 2.77-2.67 (m, 1H), 2.20 (s, 3H), 2.12 (br t, J=11.2 Hz, 2H), 1.85 (br d, J=11.2 Hz, 2H), 1.49-1.41 (m, 2H).

### Intermediate 13: Methyl 4-(2-(2-aminopyridin-3-yl)-5-phenyl-3H-imidazo[4,5-b]pyridin-3-yl)benzoate

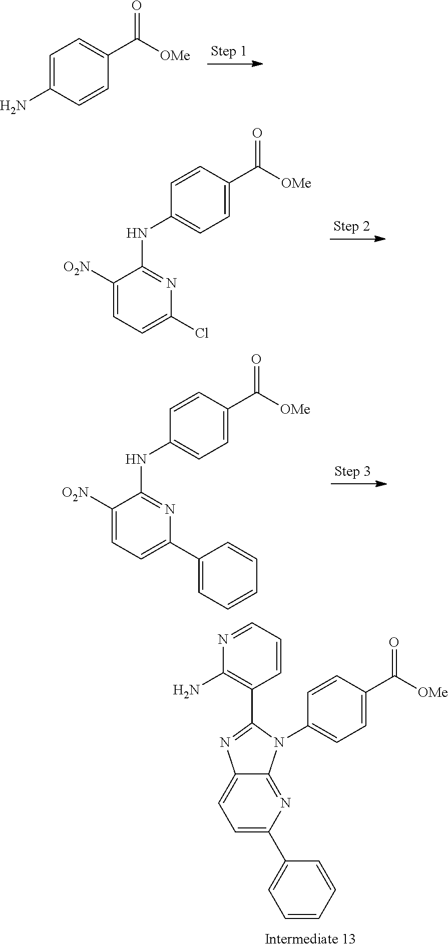

### Step 1: Methyl 4-((6-chloro-3-nitropyridin-2-yl)amino)benzoate

To a solution of methyl 4-aminobenzoate (5 g, 33.1 mmol) in DMSO (50 mL) were added 2,6-dichloro-3-nitro-pyridine (7.66 g, 39.7 mmol) and DIEA (12.82 g, 99.2 mmol). The mixture was stirred at 80° C. for 16 hr. After cooling to 20° C., the reaction mixture was poured into H₂O (100 mL) and extracted with CH₂Cl₂ (100 mL×3). The combined organic layers were washed with brine (100 mL×2), dried over anhydrous Na₂SO₄, filtered, and concentrated under reduced pressure. The crude was triturated with EtOAc at 25° C. for 30 min to give methyl 4-[(6-chloro-3-nitro-2-pyridyl)amino]benzoate (8 g, yield: 51%) as a yellow solid. MS: m/z=307.8 [M+H]⁺. ¹H NMR (400 MHz, Dimethylsulfoxide-d₆) δ 10.25 (s, 1H), 8.57 (d, J=8.4 Hz, 1H), 7.97 (d, J=8.8 Hz, 2H), 7.79 (d, J=8.8 Hz, 2H), 7.12 (d, J=8.4 Hz, 1H), 3.85 (s, 3H).

### Step 2: Methyl 4-((3-nitro-6-phenylpyridin-2-yl)amino)benzoate

To a solution of methyl 4-[(6-chloro-3-nitro-2-pyridyl)amino]benzoate (45 g, 146 mmol) and phenylboronic acid (21.4 g, 176 mmol) in 1,4-dioxane (500 mL) and H₂O (100 mL) were added Pd(dppf)Cl₂ (10.7 g, 14.6 mmol) and Cs₂CO₃ (143 g, 439 mmol). The mixture was degassed and purged with N₂ three times and stirred at 80° C. for 16 hr under N₂ atmosphere. The reaction mixture was poured into H₂O (500 mL) and extracted with CH₂Cl₂ (500 mL×3). The combined organic layers were washed with brine (500 mL×3), dried over anhydrous Na₂SO₄, filtered, and concentrated. The crude was triturated with EtOAc at 25° C. for 30 min to give methyl 4-[(3-nitro-6-phenyl-2-pyridyl)amino]benzoate (35.2 g, yield: 69%) as a red solid. MS: m/z=350.0 [M+H]⁺. ¹H NMR (400 MHz, Dimethylsulfoxide-d₆) 810.25 (s, 1H), 8.62 (d, J=8.8 Hz, 1H), 8.15-8.10 (m, 2H), 8.00 (d, J=8.4 Hz, 2H), 7.92 (d, J=8.4 Hz, 2H), 7.67 (d, J=8.8 Hz, 1H), 7.59-7.54 (m, 3H), 3.86 (s, 3H).

### Step 3: Methyl 4-(2-(2-aminopyridin-3-yl)-5-phenyl-3H-imidazo[4,5-b]pyridin-3-yl)benzoate

To a solution of methyl 4-[(3-nitro-6-phenyl-2-pyridyl)amino]benzoate (15 g, 42.9 mmol) in DMSO (150 mL) were added 2-aminopyridine-3-carbaldehyde (6.29 g, 51.5 mmol) and Na₂S₂O₄ (15 g, 85.9 mmol). The reaction mixture was heated to 100° C. for 16 hr. After cooling to 25° C., the reaction mixture was diluted with H₂O (200 mL) and extracted with CH₂Cl₂ (200 ml×3). The combined organic layers were washed with brine (200 ml×2), dried over Na₂SO₄, filtered, and concentrated under reduced pressure. The crude was triturated with CH₂Cl₂ at 25° C. for 30 min to give methyl 4-(2-(2-aminopyridin-3-yl)-5-phenyl-3H-imidazo[4,5-b]pyridin-3-yl)benzoate (Intermediate 13, 12 g, yield: 66%) as a yellow solid. MS: m/z=422.0 [M+H]⁺. ¹H NMR (400 MHz, Dimethylsulfoxide-d₆) δ 8.29 (d, J=8.0 Hz, 1H), 8.12 (d, J=8.4 Hz, 2H), 8.07-8.00 (m, 4H), 7.67 (d, J=8.8 Hz, 2H), 7.49-7.44 (m, 2H), 7.42-7.38 (m, 1H), 7.23 (dd, J=7.6, 1.6 Hz, 1H), 6.89 (br s, 2H), 6.46 (dd, J=7.6, 4.8 Hz, 1H), 3.90 (s, 3H).

### Intermediate 14: 3-(3-(4-(Chloromethyl)phenyl)-5-phenyl-3H-imidazo[4,5-b]pyridin-2-yl)pyridin-2-amine

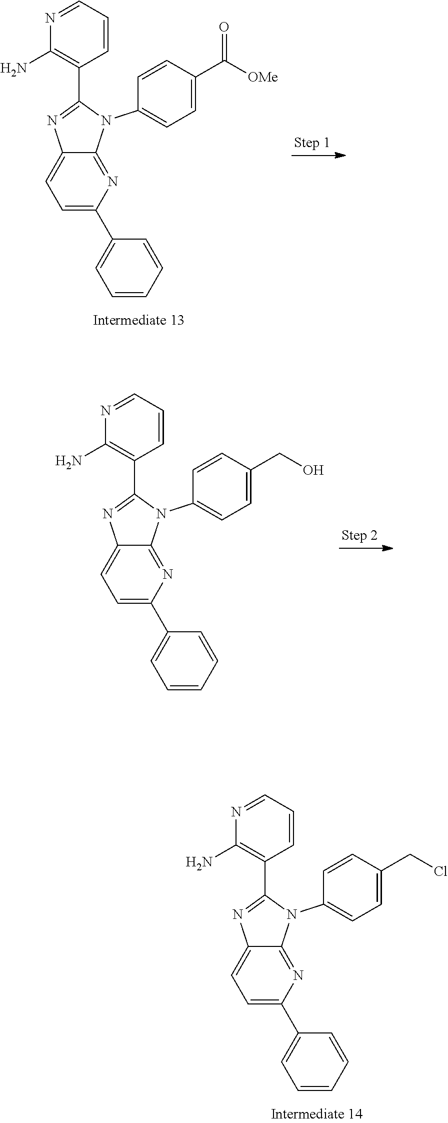 [SMILES: COC(=O)c1ccc(-n2c(-c3cccnc3N)nc3ccc(-c4ccccc4)nc32)cc1 ; Nc1ncccc1-c1nc2ccc(-c3ccccc3)nc2n1-c1ccc(CO)cc1 ; Nc1ncccc1-c1nc2ccc(-c3ccccc3)nc2n1-c1ccc(CCl)cc1]

### Step 1: (4-(2-(2-Aminopyridin-3-yl)-5-phenyl-3H-imidazo[4,5-b]pyridin-3-yl)phenyl)methanol

To a solution of Intermediate 13 (2.5 g, 5.9 mmol) in THF (25 mL) was added LiAlH₄ (450 mg, 11.9 mmol) at 0° C. After addition, the resulting mixture was stirred at 25° C. for 2 hr. After cooling to 0° C., the reaction mixture was quenched with H₂O (100 mL) and 15% aqueous NaOH (30 mL). The reaction mixture was filtered and concentrated to give [4-[2-(2-amino-3-pyridyl)-5-phenyl-imidazo[4,5-b]pyridin-3-yl]phenyl]methanol (1.87 g, yield: 80%) as a yellow solid, which was used in the next step without further purification. MS: m/z=394.1 [M+H]⁺.

### Step 2: 3-(3-(4-(Chloromethyl)phenyl)-5-phenyl-3H-imidazo[4,5-b]pyridin-2-yl)pyridin-2-amine

To a solution of [4-[2-(2-amino-3-pyridyl)-5-phenyl-imidazo[4,5-b]pyridin-3-yl]phenyl]methanol (2.3 g, 5.9 mmol) in CH₂Cl₂ (25 mL) was added SOCl₂ (2.1 g, 17.5 mmol). The mixture was stirred at 40° C. for 1 hr. The reaction mixture was filtered and concentrated to give 3-(3-(4-(Chloromethyl)phenyl)-5-phenyl-3H-imidazo[4,5-b]pyridin-2-yl)pyridin-2-amine (Intermediate 14, 1.71 g, yield: 71%) as a yellow solid. MS: m/z=412.0 [M+H]⁺. ¹H NMR (400 MHz, Dimethylsulfoxide-d₆) δ 8.29 (d, J=8.4 Hz, 1H), 8.12 (d, J=8.4 Hz, 2H), 8.07-8.00 (m, 4H), 7.67 (d, J=8.4 Hz, 2H), 7.50-7.44 (m, 2H), 7.42-7.37 (m, 1H), 7.24 (d, J=7.2 Hz, 1H), 6.88 (br s, 2H), 6.46 (dd, J=4.8, 7.6 Hz, 1H), 3.90 (s, 2H).

### Intermediate 15: 3-(3-(4-((2,6-Diazaspiro[3.3]heptan-2-yl)methyl)phenyl)-5-phenyl-3H-imidazo[4,5-b]pyridin-2-yl)pyridin-2-amine

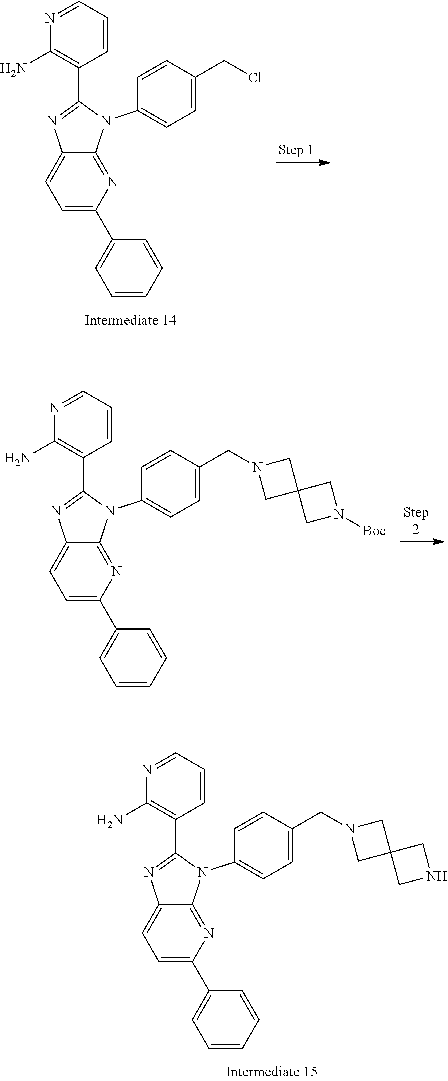 [SMILES: Nc1ncccc1-c1nc2ccc(-c3ccccc3)nc2n1-c1ccc(CCl)cc1 ; CC(C)(C)OC(=O)N1CC2(CN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC3(CNC3)C2)cc1]

### Step 1: tert-Butyl 6-(4-(2-(2-aminopyridin-3-yl)-5-phenyl-3H-imidazo[4,5-b]pyridin-3-yl)benzyl)-2,6-diazaspiro[3.3]heptane-2-carboxylate

To a solution of Intermediate 14 (200 mg, 486 μmol) in MeCN (3 mL) were added K₂CO₃ (268 mg, 1.94 mmol), tert-butyl 2,6-diazaspiro[3.3]heptane-2-carboxylate (106 mg, 534 μmol) and NaI (7.28 mg, 48 μmol) at 25° C. The reaction mixture was stirred at 80° C. for 3 hr. The reaction mixture was filtered and concentrated under reduced pressure. The residue was purified by silica gel flash chromatography (Eluent of 0-7% MeOH in CH₂Cl₂) to give tert-butyl 6-(4-(2-(2-aminopyridin-3-yl)-5-phenyl-3H-imidazo[4,5-b]pyridin-3-yl)benzyl)-2,6-diazaspiro[3.3]heptane-2-carboxylate (150 mg, yield: 54%) as a yellow solid. MS: m/z=574.6 [M+H]⁺.

### Step 2: 3-(3-(4-((2,6-Diazaspiro[3.3]heptan-2-yl)methyl)phenyl)-5-phenyl-3H-imidazo[4,5-b]pyridin-2-yl)pyridin-2-amine

To a solution of tert-butyl 6-(4-(2-(2-aminopyridin-3-yl)-5-phenyl-3H-imidazo[4,5-b]pyridin-3-yl)benzyl)-2,6-diazaspiro[3.3]heptane-2-carboxylate (150 mg, 261 μmol) in CH₂Cl₂ (3 mL) was added TFA (1.54 g, 13.5 mmol) at 25° C. The reaction mixture was stirred at 25° C. for 2 hr. The reaction mixture was concentrated to give 3-(3-(4-((2,6-diazaspiro[3.3]heptan-2-yl)methyl)phenyl)-5-phenyl-3H-imidazo[4,5-b]pyridin-2-yl)pyridin-2-amine (Intermediate 15, 120 mg, TFA salt, yield: 81%) as a yellow solid. MS: m/z=474.2 [M+H]⁺. ¹H NMR (400 MHz, Dimethylsulfoxide-d₆) δ 8.26 (d, J=8.4 Hz, 1H), 8.04-7.97 (m, 4H), 7.49-7.44 (m, 2H), 7.42-7.37 (m, 5H), 7.15 (dd, J=7.6, 1.6 Hz, 1H), 6.99 (br s, 2H), 6.39 (dd, J=8.0, 5.2 Hz, 1H), 3.58 (s, 2H), 3.56 (s, 4H), 3.25 (s, 4H), 1.23 (s, 1H).

### Intermediate 16: 3-(3-(4-((2,6-Diazaspiro[3.4]octan-2-yl)methyl)phenyl)-5-phenyl-3H-imidazo[4,5-b]pyridin-2-yl)pyridin-2-amine

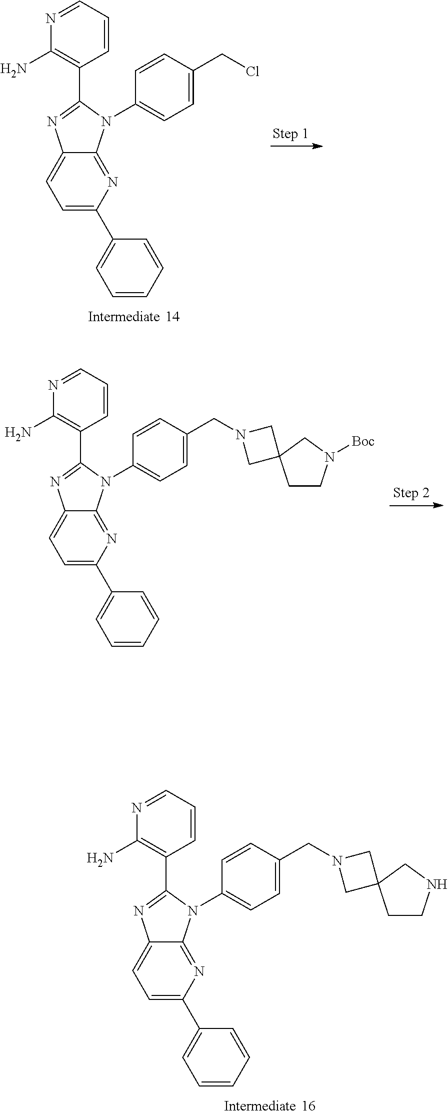

### Step 1: tert-Butyl 2-(4-(2-(2-aminopyridin-3-yl)-5-phenyl-3H-imidazo[4,5-b]pyridin-3-yl)benzyl)-2,6-diazaspiro[3.4]octane-6-carboxylate

To a solution of Intermediate 14 (200 mg, 485 μmol) in MeCN (3 mL) were added K₂CO₃ (268 mg, 1.94 mmol), tert-butyl 2,6-diazaspiro[3.4]octane-6-carboxylate (113 mg, 534 μmol) and NaI (7.28 mg, 48 μmol) at 25° C. The reaction mixture was stirred at 80° C. for 3 hr. The reaction mixture was filtered and concentrated under reduced pressure. The residue was purified by silica gel flash chromatography (Eluent of 0-7% MeOH in CH₂Cl₂) to give tert-butyl 2-(4-(2-(2-aminopyridin-3-yl)-5-phenyl-3H-imidazo[4,5-b]pyridin-3-yl)benzyl)-2,6-diazaspiro[3.4]octane-6-carboxylate (150 mg, yield: 53%) as a yellow solid. MS: m/z=588.3 [M+H]⁺.

### Step 2: 3-(3-(4-((2,6-Diazaspiro[3.4]octan-2-yl)methyl)phenyl)-5-phenyl-3H-imidazo[4,5-b]pyridin-2-yl)pyridin-2-amine

To a solution of tert-butyl 2-(4-(2-(2-aminopyridin-3-yl)-5-phenyl-3H-imidazo[4,5-b]pyridin-3-yl)benzyl)-2,6-diazaspiro[3.4]octane-6-carboxylate (150 mg, 255 μmol) in CH₂Cl₂ (3 mL) was added TFA (1.54 g, 13.5 mmol) at 25° C. The reaction mixture was stirred at 25° C. for 2 hr. The reaction mixture was poured into water (20 mL). The resulting mixture was washed with CH₂Cl₂ (50 mL×2). The pH of the aqueous phase was adjusted to 8 by NaHCO₃. The resulting mixture was extracted with CH₂Cl₂ (50 mL×2). The combined organic phases were dried over anhydrous Na₂SO₄, filtered and concentrated to give 3-(3-(4-((2,6-diazaspiro[3.4]octan-2-yl)methyl)phenyl)-5-phenyl-3H-imidazo[4,5-b]pyridin-2-yl)pyridin-2-amine (Intermediate 16, 104 mg, yield: 14%) as a yellow solid. MS: m/z=488.3 [M+H]⁺. ¹H NMR (400 MHz, Dimethylsulfoxide-d₆) δ 8.26 (d, J=8.4 Hz, 1H), 8.06-7.95 (m, 4H), 7.51-7.36 (m, 7H), 7.15 (dd, J=7.6, 1.6 Hz, 1H), 6.99 (br s, 2H), 6.39 (dd, J=7.6, 4.8 Hz, 1H), 3.63 (s, 2H), 3.16-3.10 (m, 4H), 2.88 (s, 2H), 2.74 (t, J=6.8 Hz, 2H), 1.82 (t, J=6.8 Hz, 2H), 1.25-1.22 (m, 1H).

### Intermediate 17: Benzyl (2-hydroxyethyl)(piperidin-4-yl)carbamate)

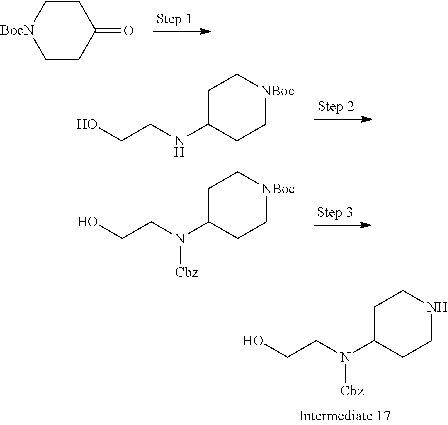

### Step 1: tert-Butyl 4-((2-hydroxyethyl)amino)piperidine-1-carboxylate)

A mixture of tert-butyl 4-oxopiperidine-1-carboxylate (20 g, 100 mmol), 2-aminoethanol (12.3 g, 201 mmol), CH₃COOH (6.0 g, 100 mmol) and Na(OAc)₃BH (53 g, 251 mmol) in CH₂Cl₂ (200 mL) was degassed and purged with N₂ three times. The mixture was stirred at 25° C. for 16 hr under N₂ atmosphere. The reaction mixture was quenched with 3 M NaOH (150 mL) at 0° C., diluted with H₂O (50 mL), and extracted with CH₂Cl₂ (100 mL×2). The combined organic layers were washed with brine (100 mL), dried over Na₂SO₄, filtered, and concentrated under reduced pressure. After purified by silica gel flash chromatography (MeOH in CH₂Cl₂=0% to 15%), tert-butyl 4-((2-hydroxyethyl)amino)piperidine-1-carboxylate (36 g, yield: 33%) was obtained as a colorless oil. ¹H NMR (400 MHz, Chloroform-d) δ 4.02 (br s, 2H), 3.62 (t, J=4.8 Hz, 2H), 2.85-2.66 (m, 4H), 2.62-2.55 (m, 1H), 2.13 (br s, 2H), 1.85-1.82 (m, 2H), 1.43 (s, 9H), 1.31-1.15 (m, 2H).

### Step 2: tert-Butyl 4-(((benzyloxy)carbonyl)(2-hydroxyethyl)amino)piperidine-1-carboxylate)

To a solution of tert-butyl 4-(2-hydroxyethylamino)piperidine-1-carboxylate (1.0 g, 4.09 mmol) and TEA (828 mg, 8.2 mmol) in CH₂Cl₂ (10 mL) was added CbzCl (698 mg, 4.09 mmol) at 0° C. The mixture was stirred at 25° C. for 16 hr. The reaction mixture was diluted with H₂O (50 mL) and extracted with EtOAc (50 mL×3). The combined organic layers were washed with brine (100 mL), dried over Na₂SO₄, filtered, and concentrated under reduced pressure. After purified by silica gel flash chromatography (EtOAc in petroleum ether=0~70%), tert-butyl 4-(((benzyloxy)carbonyl)(2-hydroxyethyl)amino)piperidine-1-carboxylate) (1.2 g, yield: 78%) was obtained as a light-yellow solid. ¹H NMR (400 MHz, Chloroform-d) δ 7.38-7.33 (m, 5H), 5.16 (s, 2H), 4.70 (d, J=6.0 Hz, 1H), 4.24-4.12 (m, 2H), 4.10-3.93 (m, 1H), 3.72-3.69 (m, 2H), 3.38-3.36 (m, 2H), 2.79-2.65 (m, 2H), 1.80-1.61 (m, 4H), 1.45 (s, 9H).

### Step 3: Benzyl (2-hydroxyethyl)(piperidin-4-yl)carbamate)

A solution of tert-butyl 4-[benzyloxycarbonyl(2-hydroxyethyl)amino]piperidine-1-carboxylate (900 mg, 2.38 mmol) in HCl/1,4-dioxane (4 M, 4 mL) was stirred at 25° C. for 2 hr. The solvent was removed under reduced pressure. Benzyl (2-hydroxyethyl)(piperidin-4-yl)carbamate) (Intermediate 17, 750 mg HCl salt, yield: 100%) was obtained as a white solid. The crude product was used in the next step without further purification. MS: m/z=279.2 [M+H]⁺.

### Intermediate 18: (R)-3-(3-(4-((3-Aminopyrrolidin-1-yl)methyl)phenyl)-5-phenyl-3H-imidazo[4,5-b]pyridin-2-yl)pyridin-2-amine

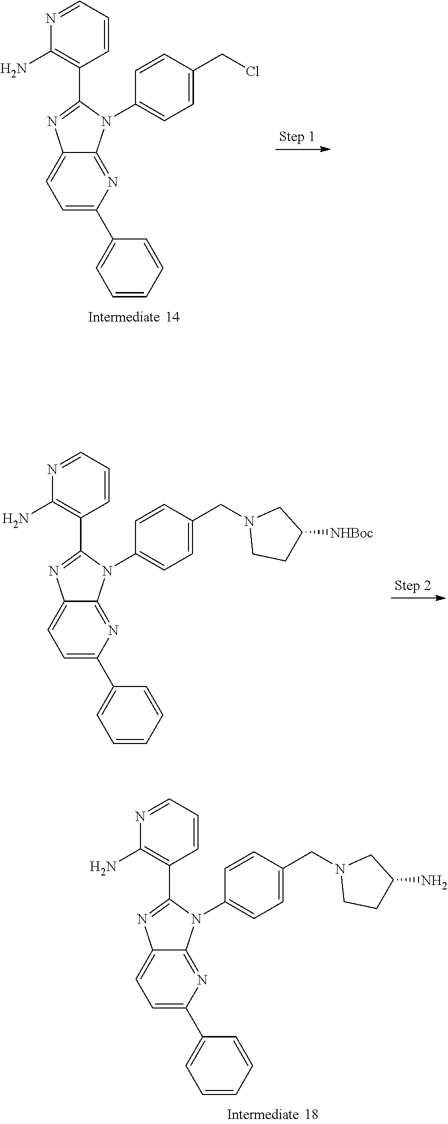

### Step 1: (R)-tert-Butyl (1-(4-(2-(2-aminopyridin-3-yl)-5-phenyl-3H-imidazo[4,5-b]pyridin-3-yl)benzyl)pyrrolidin-3-yl)carbamate

To a solution of Intermediate 14 (50 mg, 121 μmol) in DMF (0.5 mL) were added tert-butyl N-[(3R)-pyrrolidin-3-yl]carbamate (24.8 mg, 133 μmol), NaI (1.82 mg, 12.1 μmol) and K₂CO₃ (33.5 mg, 242 gmol). The mixture was stirred at 80° C. for 2 hr. The reaction mixture was diluted with H₂O (5 mL) and filtered to give (R)-tert-butyl (1-(4-(2-(2-aminopyridin-3-yl)-5-phenyl-3H-imidazo[4,5-b]pyridin-3-yl)benzyl)pyrrolidin-3-yl)carbamate (60 mg, yield: 88%) as a yellow solid, which was used in the next step without further purification. MS: m/z=562.3 [M+H]⁺.

### Step 2: (R)-3-(3-(4-((3-Aminopyrrolidin-1-yl)methyl)phenyl)-5-phenyl-3H-imidazo[4,5-b]pyridin-2-yl)pyridin-2-amine

To a solution of (R)-tert-butyl (1-(4-(2-(2-aminopyridin-3-yl)-5-phenyl-3H-imidazo[4,5-b]pyridin-3-yl)benzyl)pyrrolidin-3-yl)carbamate (60 mg, 106 μmol) in CH₂Cl₂ (2 mL) was added TFA (770 mg, 6.75 mmol). The mixture was stirred at 20° C. for 2 hr The reaction mixture was concentrated under reduced pressure to give a crude product (TFA salt). The crude was used in the next step without further purification.

The crude was quenched with sat. NaHCO₃ (5 mL) and extracted with CH₂Cl₂ (10 mL×3). The combined organic layers were washed with brine (5 ml), dried over Na₂SO₄, filtered, and concentrated under reduced pressure. The residue was purified by prep-TLC (CH₂Cl₂: MeOH=10:1) to give (R)-3-(3-(4-((3-aminopyrrolidin-1-yl)methyl)phenyl)-5-phenyl-3H-imidazo[4,5-b]pyridin-2-yl)pyridin-2-amine (Intermediate 18, 7.7 mg) as a light-yellow powder. MS: m/z=462.3 [M+H]⁺. ¹H NMR (400 MHz, Dimethylsulfoxide-d₆) δ 8.27 (d, J=8.0 Hz, 1H), 8.04-7.97 (m, 4H), 7.52-7.44 (m, 6H), 7.43-7.34 (m, 1H), 7.6 (dd, J=7.6, 2.0 Hz, 1H), 7.00 (br s, 2H), 6.40 (dd, J=8.0, 5.2 Hz, 1H), 3.69 (s, 2H), 3.60-3.57 (m, 2H), 2.75-2.66 (m, 2H), 2.46-2.40 (m, 1H), 2.18-2.08 (m, 1H), 1.66-1.56 (m, 1H).

### Intermediate 19: 3-(3-(4-((3-Aminoazetidin-1-yl)methyl)phenyl)-5-phenyl-3H-imidazo[4,5-b]pyridin-2-yl)pyridin-2-amine

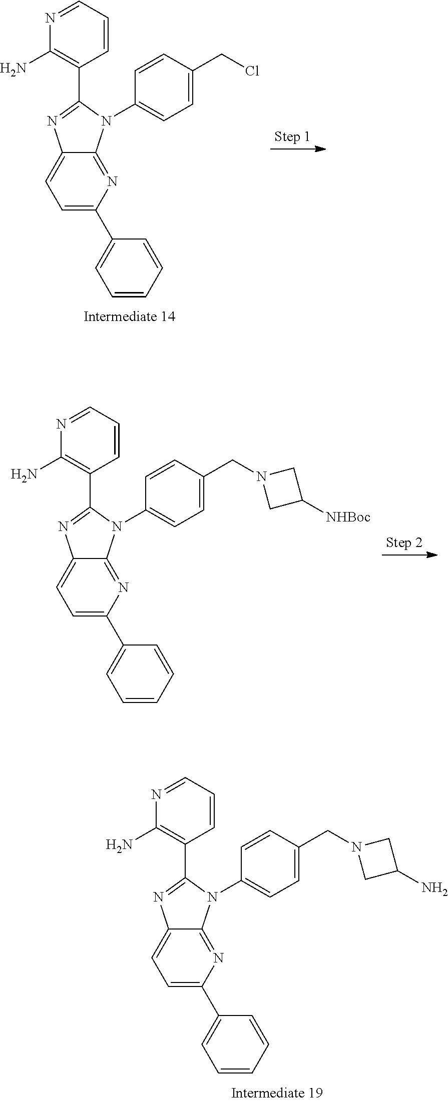 [SMILES: Nc1ncccc1-c1nc2ccc(-c3ccccc3)nc2n1-c1ccc(CCl)cc1 ; CC(C)(C)OC(=O)NC1CN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CC(N)C2)cc1]

### Step 1: tert-Butyl (1-(4-(2-(2-aminopyridin-3-yl)-5-phenyl-3H-imidazo[4,5-b]pyridin-3-yl)benzyl)azetidin-3-yl)carbamate

A solution of Intermediate 14 (50 mg, 121 μmol) in DMF (0.5 mL) were added tert-butyl N-(azetidin-3-yl)carbamate (23.0 mg, 133 μmol), NaI (1.82 mg, 12.1 μmol) and K₂CO₃ (33.5 mg, 242 mol) was stirred at 80° C. for 2 hr. The reaction mixture was diluted with H₂O (5 mL) and filtered to give tert-butyl (1-(4-(2-(2-aminopyridin-3-yl)-5-phenyl-3H-imidazo[4,5-b]pyridin-3-yl)benzyl)azetidin-3-yl)carbamate (60 mg, yield: 90%) as a yellow solid, which was used in the next step without further purification. MS: m/z=548.3 [M+H]⁺.

### Step 2: 3-(3-(4-((3-Aminoazetidin-1-yl)methyl)phenyl)-5-phenyl-3H-imidazo[4,5-b]pyridin-2-yl)pyridin-2-amine

To a solution of tert-butyl (1-(4-(2-(2-aminopyridin-3-yl)-5-phenyl-3H-imidazo[4,5-b]pyridin-3-yl)benzyl)azetidin-3-yl)carbamate (60 mg, 109 μmol) in CH₂Cl₂ (2 mL) was added TFA (770 mg, 6.75 mmol). The mixture was stirred at 20° C. for 2 hr. The reaction mixture was concentrated under reduced pressure to give 3-(3-(4-((3-aminoazetidin-1-yl)methyl)phenyl)-5-phenyl-3H-imidazo[4,5-b]pyridin-2-yl)pyridin-2-amine (Intermediate 19, 31 mg, TFA salt, yield: 53%). The crude product was used in the next step without further purification. MS: m/z=448.2 [M+H]⁺. ¹H NMR (400 MHz, Dimethylsulfoxide-d₆) δ 8.26 (d, J=8.4 Hz, 1H), 8.04-7.96 (m, 4H), 7.49-7.36 (m, 7H), 7.15 (dd, J=7.6, 1.6 Hz, 1H), 7.00 (br s, 2H), 6.39 (dd, J=8.0, 4.8 Hz, 1H), 3.61 (s, 2H), 3.52-3.48 (m, 2H), 3.47-3.38 (m, 3H), 2.72-2.65 (m, 2H).

### Intermediate 20: 2-(4-(2-(2-Aminopyridin-3-yl)-5-phenyl-3H-imidazo[4,5-b]pyridin-3-yl)benzyl)-2-azaspiro[4.5]decan-8-amine

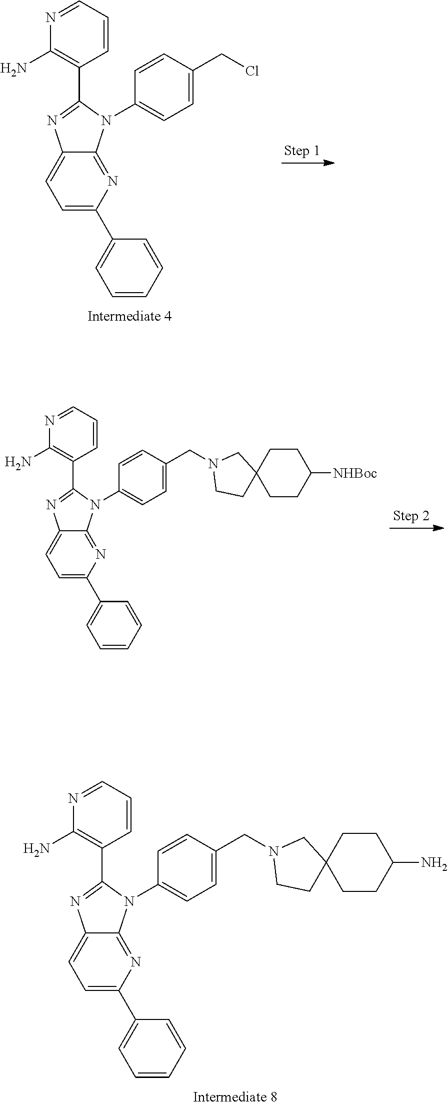 [SMILES: Nc1ncccc1-c1nc2ccc(-c3ccccc3)nc2n1-c1ccc(CCl)cc1 ; CC(C)(C)OC(=O)NC1CCC2(CC1)CCN(Cc1ccc(-n3c(-c4cccnc4N)nc4ccc(-c5ccccc5)nc43)cc1)C2 ; Nc1ncccc1-c1nc2ccc(-c3ccccc3)nc2n1-c1ccc(CN2CCC3(CCC(N)CC3)C2)cc1]

### Step 1: tert-Butyl (2-(4-(2-(2-aminopyridin-3-yl)-5-phenyl-3H-imidazo[4,5-b]pyridin-3-yl)benzyl)-2-azaspiro[4.5]decan-8-yl)carbamate

To a solution of Intermediate 14 (100 mg, 243 μmol) in CH₃CN (8 mL) were added tert-butyl 2-azaspiro[4.5]decan-8-ylcarbamate (55.6 mg, 219 μmol), NaI (3.64 mg, 24.3 μmol) and K₂CO₃ (134 mg, 971 μmol). The mixture was stirred at 80° C. for 2 hr. The mixture was concentrated under reduced pressure. The mixture was purified by silica gel flash chromatography (Eluent of 0-12% MeOH in CH₂Cl₂) to give tert-butyl (2-(4-(2-(2-aminopyridin-3-yl)-5-phenyl-3H-imidazo[4,5-b]pyridin-3-yl)benzyl)-2-azaspiro[4.5]decan-8-yl)carbamate (64 mg, yield: 42%) as a yellow solid. MS: m/z=630.5 [M+H]⁺.

### Step 2: 2-(4-(2-(2-Aminopyridin-3-yl)-5-phenyl-3H-imidazo[4,5-b]pyridin-3-yl)benzyl)-2-azaspiro[4.5]decan-8-amine

To a solution of tert-butyl (2-(4-(2-(2-aminopyridin-3-yl)-5-phenyl-3H-imidazo[4,5-b]pyridin-3-yl)benzyl)-2-azaspiro[4.5]decan-8-yl)carbamate (80 mg, 127 μmol) in CH₂Cl₂ (1 mL) was added TFA (29 mg, 254 μmol). The mixture was stirred at 25° C. for 2 hr. The mixture was diluted with H₂O (5 mL) and extracted by CH₂Cl₂ (5 mL×2). The pH of aqueous phase was adjusted to about 8 with NaHCO₃ (aq). The aqueous layer was extracted with CH₂Cl₂ (5 mL×2). The combined organic layers were washed with brine (5 mL×2), dried over Na₂SO₄, filtered and concentrated under reduced pressure to give 2-(4-(2-(2-Aminopyridin-3-yl)-5-phenyl-3H-imidazo[4,5-b]pyridin-3-yl)benzyl)-2-azaspiro[4.5]decan-8-amine (Intermediate 20, 10.9 mg, yield: 16%) as a light-yellow solid. MS: m/z=530.4 [M+H]⁺. ¹H NMR (400 MHz, Dimethylsulfoxide-d₆) δ 8.26 (d, J=8.8 Hz, 1H), 8.06-7.94 (m, 4H), 7.50-7.37 (m, 7H), 7.15 (d, J=7.2 Hz, 1H), 7.03 (br s, 2H), 6.44-6.32 (m, 1H), 4.40-4.36 (m, 1H), 4.18-4.09 (m, 6H), 1.65-1.45 (m, 6H), 1.31-1.22 (m, 4H).

### Intermediate 21: 3-(3-(4-((2,7-Diazaspiro[3.5]nonan-7-yl)methyl)phenyl)-5-phenyl-3H-imidazo[4,5-b]pyridin-2-yl)pyridin-2-amine

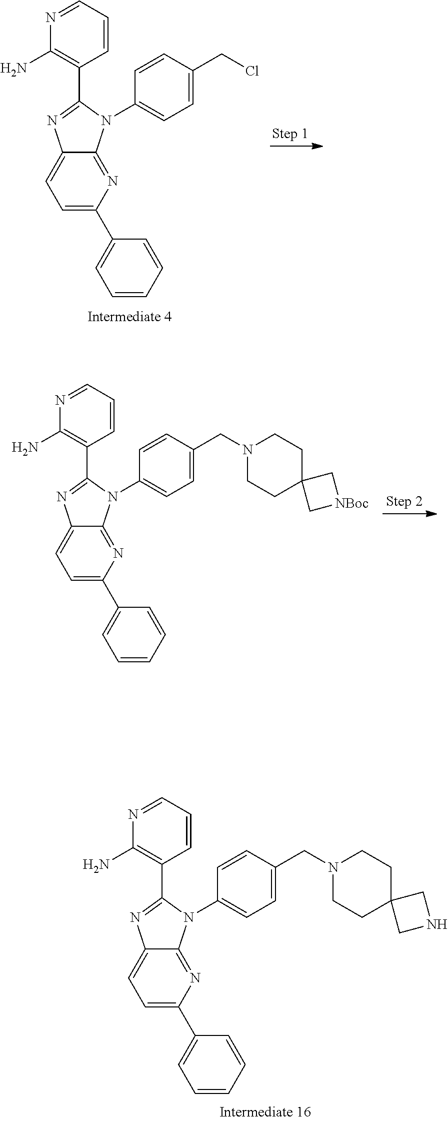 [SMILES: Nc1ncccc1-c1nc2ccc(-c3ccccc3)nc2n1-c1ccc(CCl)cc1 ; CC(C)(C)OC(=O)N1CC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CC2)CNC3)cc1]

### Step 1: tert-Butyl 7-(4-(2-(2-aminopyridin-3-yl)-5-phenyl-3H-imidazo[4,5-b]pyridin-3-yl)benzyl)-2,7-diazaspiro[3.5]nonane-2-carboxylate

To a solution of Intermediate 14 (1.0 g, 2.43 mmol) and tert-butyl 2,7-diazaspiro[3.5]nonane-2-carboxylate (660 mg, 2.92 mmol) in ACN (20 mL) were added NaI (36.4 mg, 243 μmol) and K₂CO₃ (671 mg, 4.86 mmol). The mixture was stirred at 80° C. for 2 hr. The reaction mixture was concentrated. The residue was purified by silica gel flash chromatography (Eluent of 0-10% MeOH in CH₂Cl₂) to give tert-butyl 7-(4-(2-(2-aminopyridin-3-yl)-5-phenyl-3H-imidazo[4,5-b]pyridin-3-yl)benzyl)-2,7-diazaspiro[3.5]nonane-2-carboxylate (570 mg, yield: 39%) as a light-yellow solid, which was used in the next step without further purification. MS: m/z=602.3 [M+H]⁺.

### Step 2: 3-(3-(4-((2,7-Diazaspiro[3.5]nonan-7-yl)methyl)phenyl)-5-phenyl-3H-imidazo[4,5-b]pyridin-2-yl)pyridin-2-amine

A solution of tert-butyl 7-(4-(2-(2-aminopyridin-3-yl)-5-phenyl-3H-imidazo[4,5-b]pyridin-3-yl)benzyl)-2,7-diazaspiro[3.5]nonane-2-carboxylate (490 mg, 814 μmol) in HCl in 1,4-dioxane (4M, 9 mL) was stirred at 25° C. for 1 hr. The reaction mixture was concentrated. The residue was purified by prep-HPLC (column: Waters xbridge 150*25 mm 10 m; mobile phase: [water (NH₄HCO₃)-ACN]; B %: 32%-62%, 8 min). 3-(3-(4-((2,7-Diazaspiro[3.5]nonan-7-yl)methyl)phenyl)-5-phenyl-3H-imidazo[4,5-b]pyridin-2-yl)pyridin-2-amine (Intermediate 21, 300 mg, yield: 74%) was obtained as a light-yellow oil. MS: m/z=502.3 [M+H]⁺. ¹H NMR (400 MHz, Dimethylsulfoxide-d₆) δ 8.26 (d, J=8.4 Hz, 1H), 8.05-7.97 (m, 4H), 7.49-7.39 (m, 7H), 7.18-7.12 (dd, J=7.6, 1.6 Hz, 1H), 7.02 (br s, 2H), 6.36 (dd, J=7.6, 5.2 Hz, 1H), 3.58-3.41 (m, 6H), 2.38-2.26 (m, 4H), 1.71-1.68 (m, 4H).

### Intermediate 22: 3-(3-(4-((2,8-Diazaspiro[4.5]decan-8-yl)methyl)phenyl)-5-phenyl-3H-imidazo[4,5-b]pyridin-2-yl)pyridin-2-amine

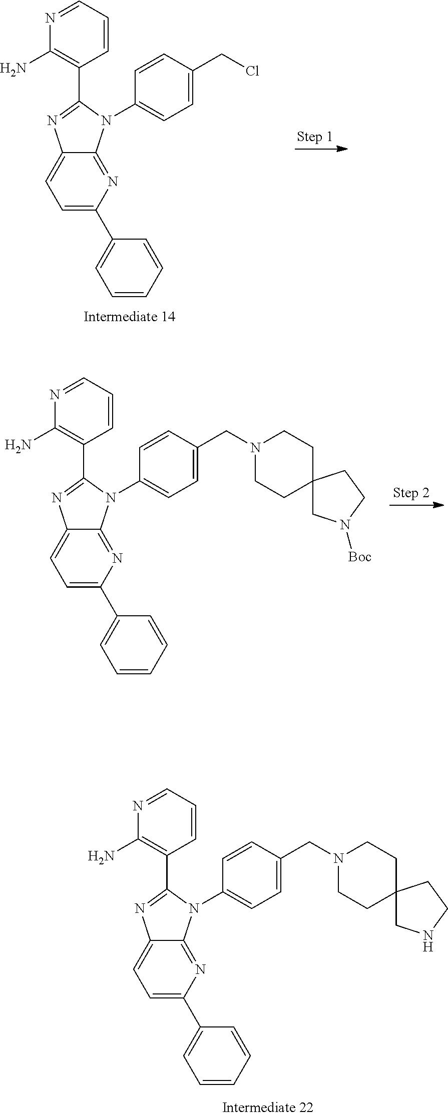 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CCNC3)CC2)cc1]

### Step 1: tert-Butyl 8-(4-(2-(2-aminopyridin-3-yl)-5-phenyl-3H-imidazo[4,5-b]pyridin-3-yl)benzyl)-2,8-diazaspiro[4.5]decane-2-carboxylate

To a solution of Intermediate 14 (250 mg, 607 μmol) in DMF (2 mL) were added tert-butyl 2,8-diazaspiro[4.5]decane-2-carboxylate (160 mg, 668 μmol), NaI (9.10 mg, 60.7 μmol) and K₂CO₃ (252 mg, 1.82 mmol). The resulting mixture was stirred at 80° C. for 16 hr. The reaction mixture was poured into H₂O (5 mL) and extracted with EtOAc (10 mL×3). The combined organic layers were dried over anhydrous Na₂SO₄, filtered, and concentrated under reduced pressure. The residue was purified by silica gel flash chromatography (Eluent of 0~65% EtOAc in petroleum ether) to give tert-butyl 8-(4-(2-(2-aminopyridin-3-yl)-5-phenyl-3H-imidazo[4,5-b]pyridin-3-yl)benzyl)-2,8-diazaspiro[4.5]decane-2-carboxylate (200 mg, yield: 53%) as a yellow solid. MS: m/z=616.2 [M+H]⁺.

### Step 2: 3-(3-(4-((2,8-Diazaspiro[4.5]decan-8-yl)methyl)phenyl)-5-phenyl-3H-imidazo[4,5-b]pyridin-2-yl)pyridin-2-amine

To a solution of tert-butyl 8-(4-(2-(2-aminopyridin-3-yl)-5-phenyl-3H-imidazo[4,5-b]pyridin-3-yl)benzyl)-2,8-diazaspiro[4.5]decane-2-carboxylate (280 mg, 455 μmol) in dioxane (1 mL) was added 4M HCl in 1,4-dioxane (2 mL). The mixture was stirred at 25° C. for 1 hr. The reaction mixture was concentrated, extracted with CH₂Cl₂ (15 mL), washed with saturated NaHCO₃ (5 mL) aqueous solution. The organic layer was separated, dried over Na₂SO₄, filtered, and concentrated under reduced pressure to give 3-(3-(4-((2,8-diazaspiro[4.5]decan-8-yl)methyl)phenyl)-5-phenyl-3H-imidazo[4,5-b]pyridin-2-yl)pyridin-2-amine (Intermediate 22, 200 mg, yield: 85%). MS: m/z=516.3 [M+H]⁺. ¹H NMR (400 MHz, Methanol-d₄) δ 8.18 (d, J=8.4 Hz, 1H), 8.02 (d, J=7.2 Hz, 2H), 7.97 (dd, J=4.8, 1.6 Hz, 1H), 7.93 (d, J=8.4 Hz, 1H), 7.52 (d, J=8.4 Hz, 2H), 7.45-7.34 (m, 5H), 7.31 (dd, J=6.8, 1.6 Hz, 1H), 6.45 (dd, J=7.6, 4.8 Hz, 1H), 3.62 (s, 2H), 3.10 (t, J=7.2 Hz, 2H), 2.85 (s, 2H), 2.50 (m, 4H), 1.75 (t, J=7.2 Hz, 2H), 1.65 (t, J=5.6 Hz, 4H).

### Intermediate 23: 3-(3-(4-((2,6-Diazaspiro[3.4]octan-6-yl)methyl)phenyl)-5-phenyl-3H-imidazo[4,5-b]pyridin-2-yl)pyridin-2-amine

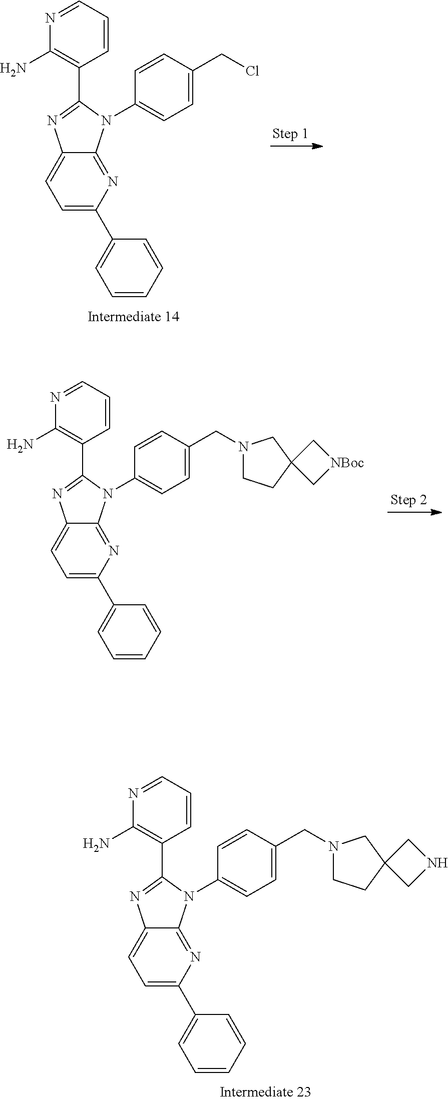 [SMILES: Nc1ncccc1-c1nc2ccc(-c3ccccc3)nc2n1-c1ccc(CCl)cc1 ; CC(C)(C)OC(=O)N1CC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CNC3)C2)cc1]

### Step 1: tert-Butyl 6-(4-(2-(2-aminopyridin-3-yl)-5-phenyl-3H-imidazo[4,5-b]pyridin-3-yl)benzyl)-2,6-diazaspiro[3.4]octane-2-carboxylate

To a solution of Intermediate 14 (200 mg, 486 μmol), tert-butyl 2,6-diazaspiro[3.4]octane-2-carboxylate (113 mg, 534 μmol) in DMF (2 mL) were added NaI (7.28 mg, 48.6 μmol) and K₂CO₃ (134 mg, 971 μmol). The mixture was stirred at 80° C. for 16 hr. The reaction mixture was poured into H₂O (5 mL) and extracted with EtOAc (10 mL×3). The combined organic layers were dried over anhydrous Na₂SO₄, filtered, and concentrated under reduced pressure. The residue was purified by silica gel flash chromatography (Eluent of 0~60% EtOAc in petroleum ether) to give tert-butyl 6-(4-(2-(2-aminopyridin-3-yl)-5-phenyl-3H-imidazo[4,5-b]pyridin-3-yl)benzyl)-2,6-diazaspiro[3.4]octane-2-carboxylate (127 mg, yield: 45%) as a yellow solid. MS: m/z=588.4 [M+H]⁺.

### Step 2: 3-(3-(4-((2,6-Diazaspiro[3.4]octan-6-yl)methyl)phenyl)-5-phenyl-3H-imidazo[4,5-b]pyridin-2-yl)pyridin-2-amine

To a solution of tert-butyl 6-(4-(2-(2-aminopyridin-3-yl)-5-phenyl-3H-imidazo[4,5-b]pyridin-3-yl)benzyl)-2,6-diazaspiro[3.4]octane-2-carboxylate (60 mg, 103 μmol) in CH₂Cl₂ (2 mL) was added TFA (47 mg, 408 μmol). The mixture was stirred at 25° C. for 1 hr. The reaction mixture was concentrated under reduced pressure to give 3-(3-(4-((2,6-diazaspiro[3.4]octan-6-yl)methyl)phenyl)-5-phenyl-3H-imidazo[4,5-b]pyridin-2-yl)pyridin-2-amine (54 mg, TFA salt, yield: 90%). Then saturated NaHCO₃ aqueous solution (10 mL) was added to adjust pH to around 9. The mixture was extracted with CH₂Cl₂ (5 mL×3). The combined organic layers were dried over Na₂SO₄, filtered and concentrated under reduced pressure to give 3-(3-(4-((2,6-diazaspiro[3.4]octan-6-yl)methyl)phenyl)-5-phenyl-3H-imidazo[4,5-b]pyridin-2-yl)pyridin-2-amine (Intermediate 23, 17.6 mg, free base) as a yellow solid. MS: m/z=488.3 [M+H]⁺. ¹H NMR (400 MHz, Methanol-d₄) δ 8.18 (d, J=8.4 Hz, 1H), 8.06-7.89 (m, 4H), 7.51 (d, J=8.0 Hz, 2H), 7.45-7.40 (m, 3H), 7.39-7.30 (m, 3H), 6.48 (dd, J=7.6, 5.2 Hz, 1H), 3.99 (d, J=2.4 Hz, 2H), 3.72 (s, 2H), 2.87 (s, 2H), 2.71-2.61 (m, 2H), 2.18 (t, J=7.2 Hz, 2H), 1.28-1.25 (m, 2H).

### Intermediate 24: 3-(3-(4-((2,7-Diazaspiro[4.4]nonan-2-yl)methyl)phenyl)-5-phenyl-3H-imidazo[4,5-b]pyridin-2-yl)pyridin-2-amine

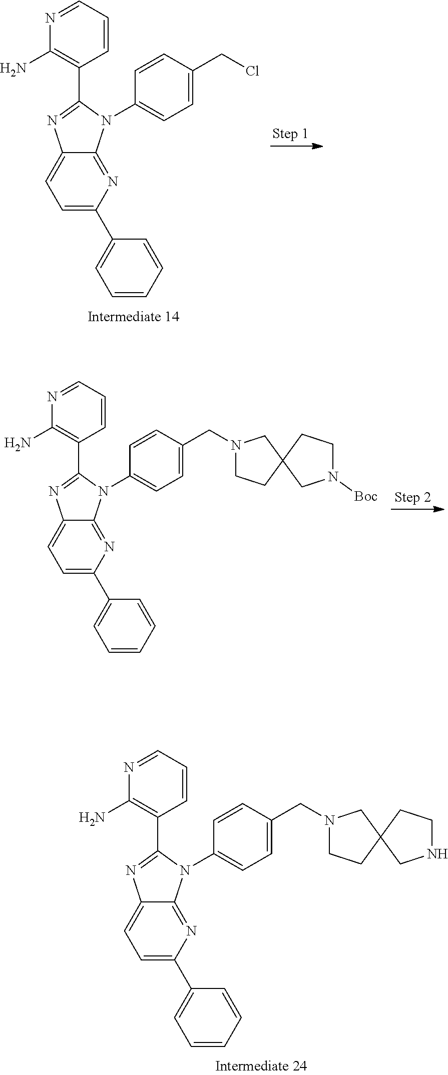 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CCNC3)C2)cc1]

### Step 1: tert-Butyl 7-(4-(2-(2-aminopyridin-3-yl)-5-phenyl-3H-imidazo[4,5-b]pyridin-3-yl)benzyl)-2,7-diazaspiro[4.4]nonane-2-carboxylate

To a solution of Intermediate 14 (1.5 g, 3.6 mmol) and tert-butyl 2,7-diazaspiro[4.4]nonane-2-carboxylate (988 mg, 4.4 mmol) in DMF (10 mL) were added NaI (273 mg, 1.82 mmol) and K₂CO₃ (1.0 g, 7.3 mmol). The mixture was stirred at 80° C. for 1 hr. Then the reaction mixture was poured into H₂O (100 mL) and extracted with EtOAc (50 mL×2). The combined organic layers were washed with brine (50 mL×2), dried over anhydrous Na₂SO₄, filtered, and concentrated to dryness. The residue was purified by silica gel flash chromatography (Eluent of 0~98% EtOAc in petroleum ether) to give tert-butyl 7-(4-(2-(2-aminopyridin-3-yl)-5-phenyl-3H-imidazo[4,5-b]pyridin-3-yl)benzyl)-2,7-diazaspiro[4.4]nonane-2-carboxylate (640 mg, yield: 30%) as a yellow solid. MS: m/z=602.4 [M+H]⁺.

### Step 2: 3-(3-(4-((2,7-Diazaspiro[4.4]nonan-2-yl)methyl)phenyl)-5-phenyl-3H-imidazo[4,5-b]pyridin-2-yl)pyridin-2-amine

A solution of tert-butyl 7-(4-(2-(2-aminopyridin-3-yl)-5-phenyl-3H-imidazo[4,5-b]pyridin-3-yl)benzyl)-2,7-diazaspiro[4.4]nonane-2-carboxylate (590 mg, 980 μmol) in HCl in 1,4-dioxane (4 M, 5 mL) was stirred at 25° C. for 0.5 hr. The mixture was concentrated, washed with CH₂Cl₂ (3 mL), and concentrated under reduced pressure to give 3-(3-(4-((2,7-diazaspiro[4.4]nonan-2-yl)methyl)phenyl)-5-phenyl-3H-imidazo[4,5-b]pyridin-2-yl)pyridin-2-amine (Intermediate 24, 452 mg, HCl salt, yield: 86%) as a yellow solid. MS: m/z=502.3 [M+H]⁺. ¹H NMR (400 MHz, Dimethylsulfoxide-d₆) δ 11.93 (br s, 1H), 9.88-9.48 (m, 2H), 8.60-8.35 (m, 2H), 8.38 (d, J=8.4 Hz, 1H), 8.16 (dd, J=6.4, 1.6 Hz, 1H), 8.08-8.05 (m, 3H), 7.98-7.83 (m, 3H), 7.66 (d, J=8.4 Hz, 2H), 7.52-7.40 (m, 3H), 7.02-6.92 (m, 1H), 4.54-4.43 (m, 2H), 3.64-3.47 (m, 2H), 3.43-3.27 (m, 3H), 3.25-3.18 (m, 3H), 2.31-2.14 (m, 2H), 2.11-1.96 (m, 2H).

### Intermediate 25: 3-(3-(4-((2,7-Diazaspiro[4.5]decan-2-yl)methyl)phenyl)-5-phenyl-3H-imidazo[4,5-b]pyridin-2-yl)pyridin-2-amine

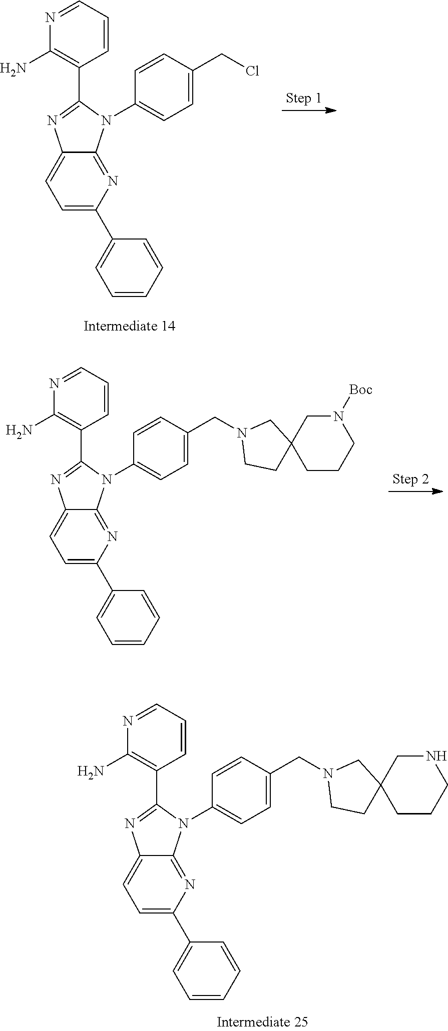 [SMILES: Nc1ncccc1-c1nc2ccc(-c3ccccc3)nc2n1-c1ccc(CCl)cc1 ; CC(C)(C)OC(=O)N1CCCC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CCCNC3)C2)cc1]

### Step 1: tert-Butyl 2-(4-(2-(2-aminopyridin-3-yl)-5-phenyl-3H-imidazo[4,5-b]pyridin-3-yl)benzyl)-2,7-diazaspiro[4.5]decane-7-carboxylate

To a solution of Intermediate 14 (1.5 g, 3.6 mmol) and tert-butyl 2,8-diazaspiro[4.5]decane-8-carboxylate (1.0 g, 4.4 mmol) in DMF (10 mL) were added NaI (273 mg, 1.8 mmol) and K₂CO₃ (1.5 g, 11 mmol). The mixture was stirred at 80° C. for 1 hr. After cooling to 20° C., the reaction mixture was poured into H₂O (100 mL) and extracted with EtOAc (50 mL×3). The combined organic layers were dried over anhydrous Na₂SO₄, filtered, and concentrated under reduced pressure. The residue was purified by silica gel flash chromatography (Eluent of 0-85% EtOAc in petroleum ether) to give tert-butyl 2-(4-(2-(2-aminopyridin-3-yl)-5-phenyl-3H-imidazo[4,5-b]pyridin-3-yl)benzyl)-2,7-diazaspiro[4.5]decane-7-carboxylate (1.2 g, yield: 53%) as a yellow solid. MS: m/z=616.1 [M+H]⁺.

### Step 2: 3-(3-(4-((2,7-Diazaspiro[4.5]decan-2-yl)methyl)phenyl)-5-phenyl-3H-imidazo[4,5-b]pyridin-2-yl)pyridin-2-amine

A solution of tert-butyl 2-(4-(2-(2-aminopyridin-3-yl)-5-phenyl-3H-imidazo[4,5-b]pyridin-3-yl)benzyl)-2,7-diazaspiro[4.5]decane-7-carboxylate (240 mg, 390 mol) in HCl/1,4-dioxane (4 M, 2 mL) was stirred at 25° C. for 0.5 hr. The mixture was concentrated and washed with CH₂Cl₂ (3 mL), then concentrated under reduced pressure. 3-(3-(4-((2,7-Diazaspiro[4.5]decan-2-yl)methyl)phenyl)-5-phenyl-3H-imidazo[4,5-b]pyridin-2-yl)pyridin-2-amine (Intermediate 25, 188 mg, HCl salt, yield: 88%) was obtained as a yellow solid. MS: m/z=516.4 [M+H]⁺. ¹H NMR (400 MHz, Methanol-d₄) δ 8.32 (d, J=8.4 Hz, 1H), 8.09-8.00 (m, 4H), 7.93 (d, J=8.4 Hz, 2H), 7.89 (d, J=7.2 Hz, 1H), 7.72 (d, J=8.0 Hz, 2H), 7.49-7.37 (m, 3H), 6.99-6.85 (m, 1H), 4.62 (d, J=11.6 Hz, 2H), 3.78-3.72 (m, 1H), 3.65-3.47 (m, 2H), 3.44-3.38 (m, 1H), 3.35 (s, 2H), 3.18-3.13 (m, 2H), 2.26-2.13 (m, 1H), 2.11-2.01 (m, 1H), 1.95-1.86 (m, 4H).

### Intermediate 26: 3-(3-(4-((2,7-Diazaspiro[3.5]nonan-2-yl)methyl)phenyl)-5-phenyl-3H-imidazo[4,5-b]pyridin-2-yl)pyridin-2-amine

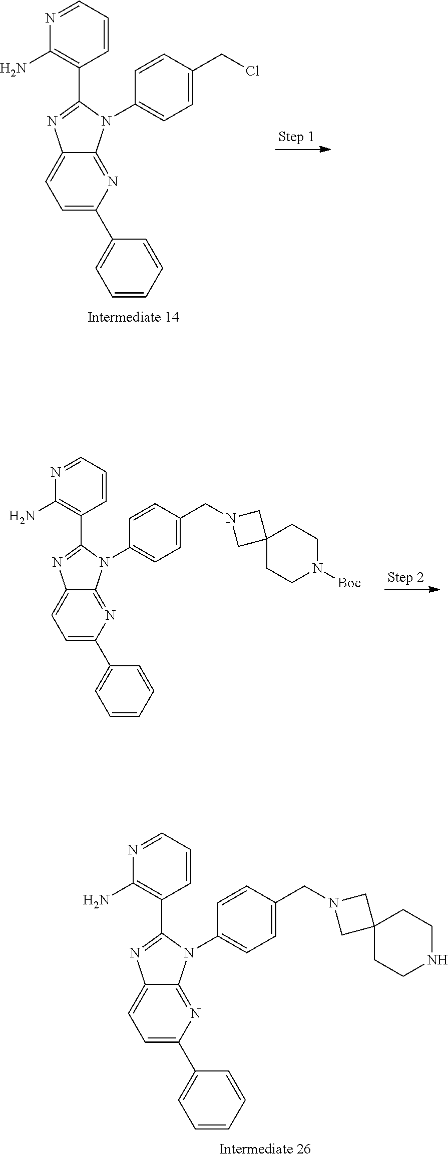 [SMILES: Nc1ncccc1-c1nc2ccc(-c3ccccc3)nc2n1-c1ccc(CCl)cc1 ; CC(C)(C)OC(=O)N1CCC2(CC1)CN(Cc1ccc(-n3c(-c4cccnc4N)nc4ccc(-c5ccccc5)nc43)cc1)C2 ; Nc1ncccc1-c1nc2ccc(-c3ccccc3)nc2n1-c1ccc(CN2CC3(CCNCC3)C2)cc1]

### Step 1: tert-Butyl 2-(4-(2-(2-aminopyridin-3-yl)-5-phenyl-3H-imidazo[4,5-b]pyridin-3-yl)benzyl)-2,7-diazaspiro[3.5]nonane-7-carboxylate

To a solution of Intermediate 14 (1.0 g, 2.43 mmol) in MeCN (10 mL) were added K₂CO₃ (1.0 g, 7.28 mmol), NaI (109 mg, 728 μmol), and tert-butyl 2,7-diazaspiro[3.5]nonane-7-carboxylate (604 mg, 2.67 mmol). The mixture was stirred at 25° C. for 16 hr. The reaction mixture was diluted with H₂O (10 mL) at 25° C. and extracted with CH₂Cl₂ (20 mL×3). The combined organic layers were washed with brine (30 mL), dried over Na₂SO₄, filtered, and concentrated under reduced pressure. The crude was purified by silica gel flash chromatography (Eluent of 1~5% MeOH in CH₂Cl₂). tert-Butyl 2-(4-(2-(2-aminopyridin-3-yl)-5-phenyl-3H-imidazo[4,5-b]pyridin-3-yl)benzyl)-2,7-diazaspiro[3.5]nonane-7-carboxylate (775 mg, yield: 53%) was obtained as a yellow solid. MS: m/z=602.4 [M+H]⁺. ¹H NMR (400 MHz, Dimethylsulfoxide-d₆) δ 8.26 (d, J=8.4 Hz, 1H), 8.05-7.96 (m, 4H), 7.49-7.39 (m, 7H), 7.16 (dd, J=7.6, 1.6 Hz, 1H), 6.99 (br s, 2H), 6.39 (dd, J=7.6, 4.8 Hz, 1H), 3.68 (s, 2H), 3.26-3.22 (m, 4H), 3.01 (s, 4H), 1.64-1.58 (m, 4H), 1.38 (s, 9H).

### Step 2: 3-(3-(4-((2,7-Diazaspiro[3.5]nonan-2-yl)methyl)phenyl)-5-phenyl-3H-imidazo[4,5-b]pyridin-2-yl)pyridin-2-amine

A solution of tert-butyl 2-(4-(2-(2-aminopyridin-3-yl)-5-phenyl-3H-imidazo[4,5-b]pyridin-3-yl)benzyl)-2,7-diazaspiro[3.5]nonane-7-carboxylate (350 mg, 582 μmol) in 4 M HCl in 1,4-dioxane (4 mL) was stirred at 25° C. for 2 hr. The reaction was concentrated under reduced pressure to give 3-(3-(4-((2,7-diazaspiro[3.5]nonan-2-yl)methyl)phenyl)-5-phenyl-3H-imidazo[4,5-b]pyridin-2-yl)pyridin-2-amine (Intermediate 26, 270 mg, HCl salt, yield: 86%) as a yellow solid. MS: m/z=502.3 [M+H]⁺. ¹H NMR (400 MHz, Dimethylsulfoxide-d₆) δ 11.79-11.63 (m, 1H), 9.00-8.82 (m, 2H), 8.36 (d, J=8.8 Hz, 1H), 8.33-8.18 (m, 1H), 8.13 (dd, J=6.8, 1.2 Hz, 1H), 8.04-8.08 (m, 3H), 7.83-7.75 (m, 3H), 7.65 (d, J=8.4 Hz, 2H), 7.52-7.46 (m, 2H), 7.45-7.39 (m, 1H), 6.92-6.83 (m, 1H), 4.50 (d, J=6.0 Hz, 2H), 3.96 (d, J=6.0 Hz, 4H), 3.07-3.10 (m, 2H), 2.95-3.02 (m, 2H), 2.10-2.17 (m, 2H), 1.97-2.04 (m, 2H).

### Intermediate 27: (R)-3-(3-(4-((2,7-Diazaspiro[4.4]nonan-2-yl)methyl)phenyl)-5-phenyl-3H-imidazo[4,5-b]pyridin-2-yl)pyridin-2-amine

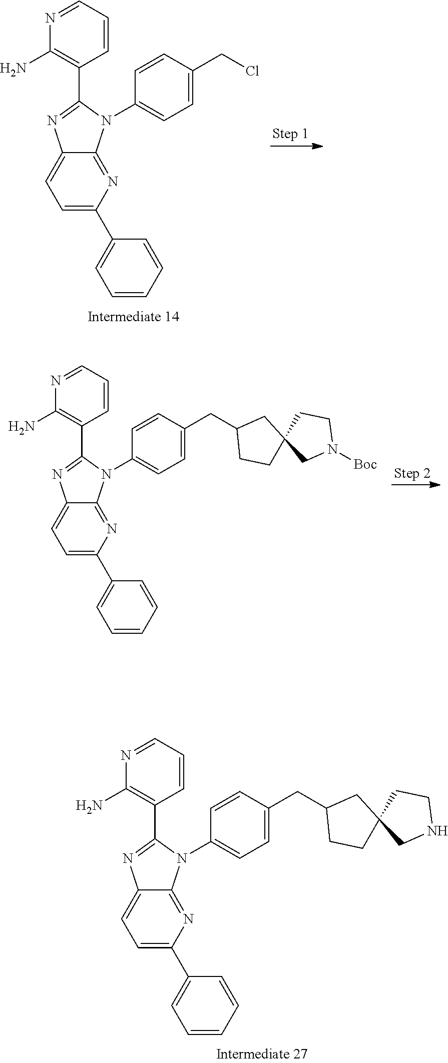 [SMILES: Nc1ncccc1-c1nc2ccc(-c3ccccc3)nc2n1-c1ccc(CCl)cc1 ; CC(C)(C)OC(=O)N1CC[C@@]2(CCC(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CC2CC[C@@]3(CCNC3)C2)cc1]

### Step 1: tert-Butyl (R)-7-(4-(2-(2-aminopyridin-3-yl)-5-phenyl-3H-imidazo[4,5-b]pyridin-3-yl)benzyl)-2,7-diazaspiro[4.4]nonane-2-carboxylate

To a solution of Intermediate 14 (1.0 g, 2.43 mmol) in DMF (10 mL) were added K₂CO₃ (671 mg, 4.86 mmol) and tert-butyl (R)-2,7-diazaspiro[4.4]nonane-2-carboxylate (604 mg, 2.67 mmol). The mixture was stirred at 25° C. for 16 hr. The reaction mixture was diluted with H₂O (10 mL) at 25° C. and extracted with CH₂Cl₂ (20 mL×3). The combined organic layers were washed with brine (30 mL), dried over Na₂SO₄, filtered, and concentrated under reduced pressure. The crude product was purified by silica gel flash chromatography (Eluent of 1-5% MeOH in CH₂Cl₂) to give tert-butyl (R)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (920 mg, yield: 63%) as a yellow solid. MS: m/z=602.6 [M+H]⁺.

### Step 2: (R)-3-(3-(4-((2,7-Diazaspiro[4.4]nonan-2-yl)methyl)phenyl)-5-phenyl-3H-imidazo[4,5-b]pyridin-2-yl)pyridin-2-amine

A solution of tert-butyl (R)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (700 mg, 1.16 mmol) in 4 M HCl and 1,4-dioxane (7 mL) was stirred at 25° C. for 2 hr. The reaction was concentrated under reduced pressure to give (R)-3-(3-(4-((2,7-diazaspiro[4.4]nonan-2-yl)methyl)phenyl)-5-phenyl-3H-imidazo[4,5-b]pyridin-2-yl)pyridin-2-amine (Intermediate 27, 600 mg, HCl salt, yield: 96%) as a yellow solid. MS: m/z=502.3 [M+H]⁺. ¹H NMR (400 MHz, Dimethylsulfoxide-d₆) δ 12.00-11.77 (m, 1H), 9.73-9.52 (m, 2H), 8.58-8.44 (m, 1H), 8.38 (d, J=8.4 Hz, 1H), 8.16 (dd, J=6.0, 1.6 Hz, 1H), 8.04-8.09 (m, 3H), 7.92-7.85 (m, 3H), 7.66 (d, J=8.4 Hz, 2H), 7.52-7.46 (m, 2H), 7.45-7.40 (m, 1H), 7.00-6.92 (m, 1H), 4.57-4.41 (m, 2H), 3.50-3.58 (m, 4H), 3.41-3.33 (m, 2H), 3.25-3.22 (m, 2H), 2.28-2.13 (m, 2H), 2.10-1.97 (m, 2H).

### Intermediate 28: 3-(3-(4-((2,5-Diazabicyclo[2.2.2]octan-2-yl)methyl)phenyl)-5-phenyl-3H-imidazo[4,5-b]pyridin-2-yl)pyridin-2-amine

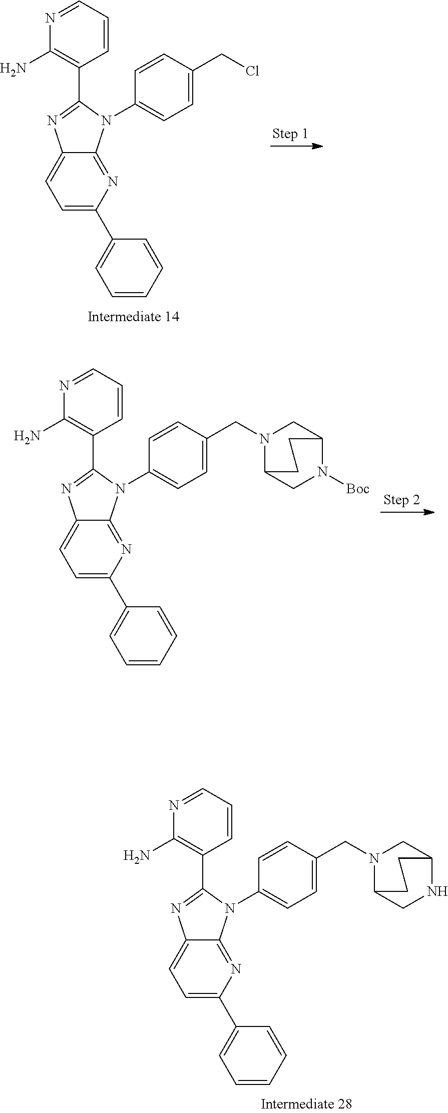 [SMILES: Nc1ncccc1-c1nc2ccc(-c3ccccc3)nc2n1-c1ccc(CCl)cc1 ; CC(C)(C)OC(=O)N1CC2CCC1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2CC3CCC2CN3)cc1]

### Step 1: tert-Butyl 5-(4-(2-(2-aminopyridin-3-yl)-5-phenyl-3H-imidazo[4,5-b]pyridin-3-yl)benzyl)-2,5-diazabicyclo[2.2.2]octane-2-carboxylate

To a solution of Intermediate 14 (1.0 g, 2.43 mmol) and tert-butyl 2,5-diazabicyclo[2.2.2]octane-2-carboxylate (567 mg, 2.67 mmol) in MeCN (10 mL) were added K₂CO₃ (1.0 mg, 7.28 mmol) and NaI (109 mg, 728 μmol). The mixture was stirred at 25° C. for 16 hr. The reaction mixture was diluted with H₂O (10 mL) at 25° C. and extracted with CH₂Cl₂ (10 mL×3). The combined organic layers were washed with brine (30 mL), dried over Na₂SO₄, filtered, and concentrated under reduced pressure. The crude was purified by silica gel flash chromatography (Eluent of 0-5% MeOH in CH₂Cl₂) to give tert-butyl 5-(4-(2-(2-aminopyridin-3-yl)-5-phenyl-3H-imidazo[4,5-b]pyridin-3-yl)benzyl)-2,5-diazabicyclo[2.2.2]octane-2-carboxylate (700 mg, yield: 45%) as a yellow solid. MS: m/z=588.3 [M+H]⁺. ¹H NMR (400 MHz, Dimethylsulfoxide-d₆) δ 8.26 (d, J=8.4 Hz, 1H), 8.06-7.95 (m, 4H), 7.54-7.36 (m, 7H), 7.15 (dd, J=8.0, 1.6 Hz, 1H), 7.02 (s, 2H), 6.38 (dd, J=7.6, 4.8 Hz, 1H), 3.94-3.72 (m, 3H), 3.69-3.54 (m, 1H), 3.27-3.20 (m, 1H), 3.17 (d, J=5.2 Hz, 0.0.5H), 2.90-2.86 (m, 0.5H), 2.84-2.79 (m, 2H), 2.07-1.95 (m, 1H), 1.78-1.69 (m, 2H), 1.63-1.52 (m, 1H), 1.45-1.37 (d, J=9.6 Hz, 9H).

### Step 2: 3-(3-(4-((2,5-Diazabicyclo[2.2.2]octan-2-yl)methyl)phenyl)-5-phenyl-3H-imidazo[4,5-b]pyridin-2-yl)pyridin-2-amine

A solution of tert-butyl 5-(4-(2-(2-aminopyridin-3-yl)-5-phenyl-3H-imidazo[4,5-b]pyridin-3-yl)benzyl)-2,5-diazabicyclo[2.2.2]octane-2-carboxylate (50 mg, 85 μmol) in 4 M HCl in 1,4-dioxane (1 mL) was stirred at 25° C. for 2 hr. The mixture was filtered. The collected solid was washed with 1,4-dioxane (10 mL×2) and dried under reduced pressure to give 3-(3-(4-((2,5-diazabicyclo[2.2.2]octan-2-yl)methyl)phenyl)-5-phenyl-3H-imidazo[4,5-b]pyridin-2-yl)pyridin-2-amine (Intermediate 28, 44.9 mg, yield: 99%) as a yellow solid. MS: m/z=488.2 [M+H]⁺. ¹H NMR (400 MHz, Dimethylsulfoxide-d₆) δ 12.11-11.44 (m, 1H), 10.27-9.61 (m, 2H), 8.56-8.40 (m, 1H), 8.38 (d, J=8.4 Hz, 1H), 8.15 (dd, J=6.0, 1.2 Hz, 1H), 8.10-7.99 (m, 5H), 7.86 (dd, J=7.2, 1.2 Hz, 1H), 7.68 (d, J=8.4 Hz, 2H), 7.51-7.46 (m, 2H), 7.46-7.40 (m, 1H), 6.91 (dd, J=7.6, 6.4 Hz, 1H), 4.62 (br s, 2H), 4.01-3.79 (m, 4H), 3.76-3.70 (m, 2H), 2.21 (m, 2H), 1.97-1.80 (m, 2H).

### Intermediate 29: 3-(3-(4-((2,6-Diazaspiro[3.5]nonan-2-yl)methyl)phenyl)-5-phenyl-3H-imidazo[4,5-b]pyridin-2-yl)pyridin-2-amine

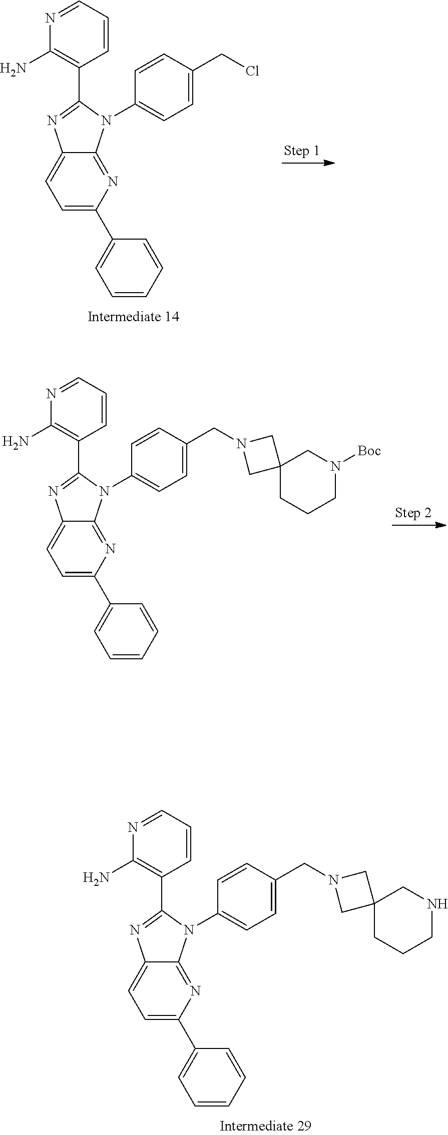 [SMILES: Nc1ncccc1-c1nc2ccc(-c3ccccc3)nc2n1-c1ccc(CCl)cc1 ; CC(C)(C)OC(=O)N1CCCC2(CN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC3(CCCNC3)C2)cc1]

### Step 1: tert-butyl 2-(4-(2-(2-aminopyridin-3-yl)-5-phenyl-3H-imidazo[4,5-b]pyridin-3-yl)benzyl)-2,6-diazaspiro[3.5]nonane-6-carboxylate

To a solution of Intermediate 14 (1.1 g, 2.67 mmol) and tert-butyl 2,6-diazaspiro[3.5]nonane-6-carboxylate (725 mg, 3.20 mmol) in MeCN (15 mL) were added K₂CO₃ (1.11 g, 8.01 mmol) and NaI (120 mg, 801 μmol). The mixture was stirred at 25° C. for 16 hr. The reaction mixture was diluted with H₂O (30 mL) at 25° C. and extracted with EtOAc (50 mL×3). The combined organic layers were washed with brine (100 mL), dried over Na₂SO₄, filtered, and concentrated under reduced pressure. The crude was purified by silica gel flash chromatography (Eluent of 1~4% MeOH in CH₂Cl₂) to give tert-butyl 2-(4-(2-(2-aminopyridin-3-yl)-5-phenyl-3H-imidazo[4,5-b]pyridin-3-yl)benzyl)-2,6-diazaspiro[3.5]nonane-6-carboxylate (900 mg, yield: 56%) as a yellow solid. MS: m/z=602.4 [M+H]⁺. ¹H NMR (400 MHz, Dimethylsulfoxide-d₆) δ 8.26 (d, J=8.4 Hz, 1H), 8.05-7.95 (m, 4H), 7.50-7.35 (m, 6H), 7.15 (dd, J=7.6, 1.6 Hz, 1H), 7.01 (br s, 2H), 6.36 (dd, J=7.6, 4.8 Hz, 1H), 3.69 (s, 2H), 3.45 (s, 2H), 3.30-3.20 (m, 2H), 3.10-3.00 (m, 2H), 2.87-2.75 (m, 2H), 2.52-2.50 (m, 2H), 1.70-1.62 (m, 2H), 1.49-1.41 (m, 2H), 1.38 (s, 9H).

### Step 2: 3-(3-(4-((2,6-Diazaspiro[3.5]nonan-2-yl)methyl)phenyl)-5-phenyl-3H-imidazo[4,5-b]pyridin-2-yl)pyridin-2-amine

A solution of tert-butyl 2-(4-(2-(2-aminopyridin-3-yl)-5-phenyl-3H-imidazo[4,5-b]pyridin-3-yl)benzyl)-2,6-diazaspiro[3.5]nonane-6-carboxylate (50 mg, 83.1 μmol) in 4 M HCl in 1,4-dioxane (5 mL) at 25° C. was stirred at 25° C. for 4 hr. The reaction was filtered and concentrated under reduced pressure to give 3-(3-(4-((2,6-diazaspiro[3.5]nonan-2-yl)methyl)phenyl)-5-phenyl-3H-imidazo[4,5-b]pyridin-2-yl)pyridin-2-amine (Intermediate 29, 37 mg, HCl salt, yield: 84%) as a yellow solid. MS: m/z=502.3 [M+H]⁺. ¹H NMR (400 MHz, Dimethylsulfoxide-d₆) δ 8.26 (d, J=8.4 Hz, 1H), 8.03-7.97 (m, 5H), 7.50-7.39 (m, 6H), 7.15 (dd, J=7.6, 1.6 Hz, 1H), 7.01 (br s, 2H), 6.36 (dd, J=7.6, 4.8 Hz, 1H), 3.66 (s, 2H), 3.04 (d, J=7.2 Hz, 2H), 2.85 (d, J=6.8 Hz, 2H), 2.78 (s, 2H), 2.62-2.58 (m, 2H), 1.64-1.60 (m, 2H), 1.40-1.35 (m, 2H).

### Intermediate 30: (S)-3-(3-(4-((2,7-Diazaspiro[4.4]nonan-2-yl)methyl)phenyl)-5-phenyl-3H-imidazo[4,5-b]pyridin-2-yl)pyridin-2-amine

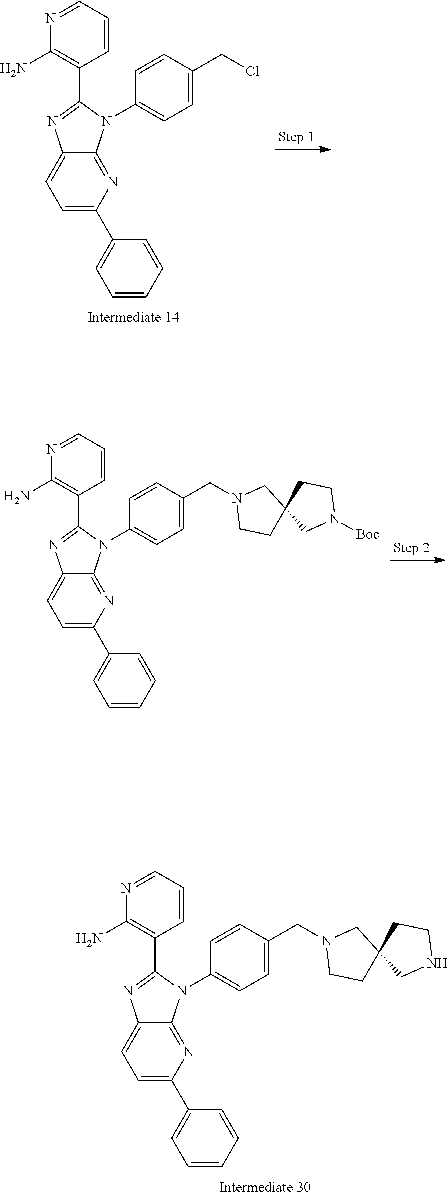 [SMILES: Nc1ncccc1-c1nc2ccc(-c3ccccc3)nc2n1-c1ccc(CCl)cc1 ; CC(C)(C)OC(=O)N1CC[C@]2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC[C@]3(CCNC3)C2)cc1]

### Step 1: tert-Butyl (S)-7-(4-(2-(2-aminopyridin-3-yl)-5-phenyl-3H-imidazo[4,5-b]pyridin-3-yl)benzyl)-2,7-diazaspiro[4.4]nonane-2-carboxylate

To a solution of Intermediate 14 (1.0 g, 2.43 mmol) and tert-butyl (S)-2,7-diazaspiro[4.4]nonane-2-carboxylate (659 mg, 2.91 mmol) in DMF (15 mL) was added K₂CO₃ (671 mg, 4.86 mmol). The mixture was stirred at 25° C. for 16 hr. The reaction mixture was diluted with H₂O (50 mL) at 25° C. and extracted with EtOAc (100 mL×2). The combined organic layers were washed with brine (200 mL), dried over Na₂SO₄, filtered, and concentrated under reduced pressure. The residue was purified by silica gel flash chromatography (Eluent of 0-5% MeOH in CH₂Cl₂) to give tert-butyl (S)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (600 mg, yield: 41%) as a brown solid. MS: m/z=602.3 [M+H]⁺. ¹H NMR (400 MHz, Chloroform-d) δ 8.13 (d, J=8.4 Hz, 1H), 8.07 (dd, J=4.8, 1.6 Hz, 1H), 8.03-8.01 (m, 2H), 7.8 (d, J=8.4 Hz, 1H), 7.51-7.49 (m, 2H), 7.46-7.35 (m, 5H), 7.09 (dd, J=7.6, 1.6 Hz, 1H), 6.59 (br s, 2H), 6.36 (dd, J=7.6, 4.8 Hz, 1H), 3.80-3.70 (m, 2H), 3.50-3.20 (m, 4H), 2.80-2.50 (m, 4H), 1.95-1.80 (m, 4H), 1.46 (s, 9H).

### Step 2: (S)-3-(3-(4-((2,7-Diazaspiro[4.4]nonan-2-yl)methyl)phenyl)-5-phenyl-3H-imidazo[4,5-b]pyridin-2-yl)pyridin-2-amine

To a solution of tert-butyl (S)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (580 mg, 964 μmol) in 1,4-dioxane (2 mL) was added HCl/1,4-dioxane (4M, 8 mL). The mixture was stirred at 25° C. for 2 hr. The reaction was filtered and concentrated under reduced pressure. (S)-3-(3-(4-((2,7-diazaspiro[4.4]nonan-2-yl)methyl)phenyl)-5-phenyl-3H-imidazo[4,5-b]pyridin-2-yl)pyridin-2-amine (Intermediate 30, 500 mg, HCl salt, yield: 96%) was obtained as a yellow solid. MS: m/z=502.3 [M+H]⁺. ¹H NMR (400 MHz, Dimethylsulfoxide-d₆) δ 11.9 (br s, 1H), 9.80-9.66 (m, 2H), 8.66-8.50 (m, 2H), 8.38 (d, J=8.4 Hz, 1H), 8.17 (dd, J=6.4, 1.6 Hz, 1H), 8.08-8.05 (m, 3H), 7.93-7.86 (M, 3H), 7.66 (d, J=8.4 Hz, 2H), 7.54-7.40 (m, 3H), 7.00-6.95 (m, 1H), 3.55-3.50 (m, 1H), 3.40-3.17 (m, 8H), 2.33-1.95 (m, 5H).

### Intermediate 31: (R)-3-(3-(4-((3-Methylpiperazin-1-yl)methyl)phenyl)-5-phenyl-3H-imidazo[4,5-b]pyridin-2-yl)pyridin-2-amine

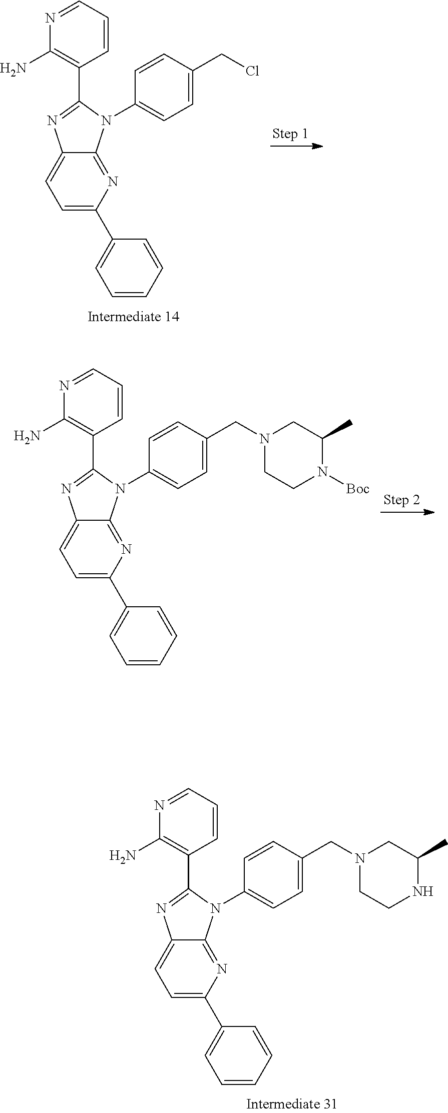 [SMILES: Nc1ncccc1-c1nc2ccc(-c3ccccc3)nc2n1-c1ccc(CCl)cc1 ; C[C@@H]1CN(Cc2ccc(-n3c(-c4cccnc4N)nc4ccc(-c5ccccc5)nc43)cc2)CCN1C(=O)OC(C)(C)C ; C[C@@H]1CN(Cc2ccc(-n3c(-c4cccnc4N)nc4ccc(-c5ccccc5)nc43)cc2)CCN1]

### Step 1: (R)-tert-Butyl 4-(4-(2-(2-aminopyridin-3-yl)-5-phenyl-3H-imidazo[4,5-b]pyridin-3-yl)benzyl)-2-methylpiperazine-1-carboxylate

To a solution of Intermediate 14 (1.0 g, 2.43 mmol) in DMF (5 mL) were added K₂CO₃ (671 mg, 4.86 mmol) and (R)-tert-butyl 2-methylpiperazine-1-carboxylate (632 mg, 3.16 mmol). The mixture was stirred at 25° C. for 16 hr. The reaction mixture was diluted with H₂O (10 mL) at 25° C. and extracted with CH₂Cl₂ (20 mL×3). The combined organic layers were washed with brine (30 mL), dried over Na₂SO₄, filtered, and concentrated under reduced pressure. The crude was purified by silica gel flash chromatography (Eluent of 1~4% MeOH in CH₂Cl₂) to give (R)-tert-butyl 4-(4-(2-(2-aminopyridin-3-yl)-5-phenyl-3H-imidazo[4,5-b]pyridin-3-yl)benzyl)-2-methylpiperazine-1-carboxylate (671 mg, yield: 48%) as a yellow solid. MS: m/z=576.3 [M+H]⁺. ¹H NMR (400 MHz, Dimethylsulfoxide-d₆) δ 8.27 (d, J=8.4 Hz, 1H), 8.04-7.95 (m, 4H), 7.51-7.38 (m, 7H), 7.16 (dd, J=7.6, 1.6 Hz, 1H), 7.04 (br s, 2H), 6.38 (dd, J=8.0, 4.8 Hz, 1H), 4.14-4.07 (m, 1H), 3.72-3.70 (m, 1H), 3.64 (d, J=13.6 Hz, 1H), 3.49 (d, J=13.6 Hz, 1H), 3.09-2.98 (m, 1H), 2.84-2.78 (m, 1H), 2.63-2.57 (m, 1H), 2.08 (dd, J=11.2, 4.0 Hz, 1H), 2.02-1.92 (m, 1H), 1.40 (s, 9H), 1.17 (d, J=6.8 Hz, 3H).

### Step 2: (R)-3-(3-(4-((3-Methylpiperazin-1-yl)methyl)phenyl)-5-phenyl-3H-imidazo[4,5-b]pyridin-2-yl)pyridin-2-amine

A solution of (R)-tert-butyl 4-(4-(2-(2-aminopyridin-3-yl)-5-phenyl-3H-imidazo[4,5-b]pyridin-3-yl)benzyl)-2-methylpiperazine-1-carboxylate (700 mg, 1.22 mmol) in 4 M HCl in 1,4-dioxane (5 mL) was stirred at 25° C. for 2 hr. The reaction was concentrated under reduced pressure to give product (R)-3-(3-(4-((3-Methylpiperazin-1-yl)methyl)phenyl)-5-phenyl-3H-imidazo[4,5-b]pyridin-2-yl)pyridin-2-amine (Intermediate 31, 500 mg, HCl salt) as a yellow solid. MS: m/z=476.2 [M+H]⁺. ¹H NMR (400 MHz, Dimethylsulfoxide-d₆) δ 8.27 (d, J=8.4 Hz, 1H), 8.08-7.95 (m, 4H), 7.52-7.35 (m, 7H), 7.15 (d, J=6.0 Hz, 1H), 7.04 (br s, 2H), 6.40-6.34 (m, 1H), 3.53 (s, 2H), 2.87-2.77 (m, 1H), 2.75-2.62 (m, 4H), 2.02-1.91 (m, 1H), 1.68-1.58 (m, 1H), 0.92 (d, J=6.0 Hz, 3H).

### Intermediate 32: 3-(3-(4-(((3S,5R)-3,5-Dimethylpiperazin-1-yl)methyl)phenyl)-5-phenyl-3H-imidazo[4,5-b]pyridin-2-yl)pyridin-2-amine

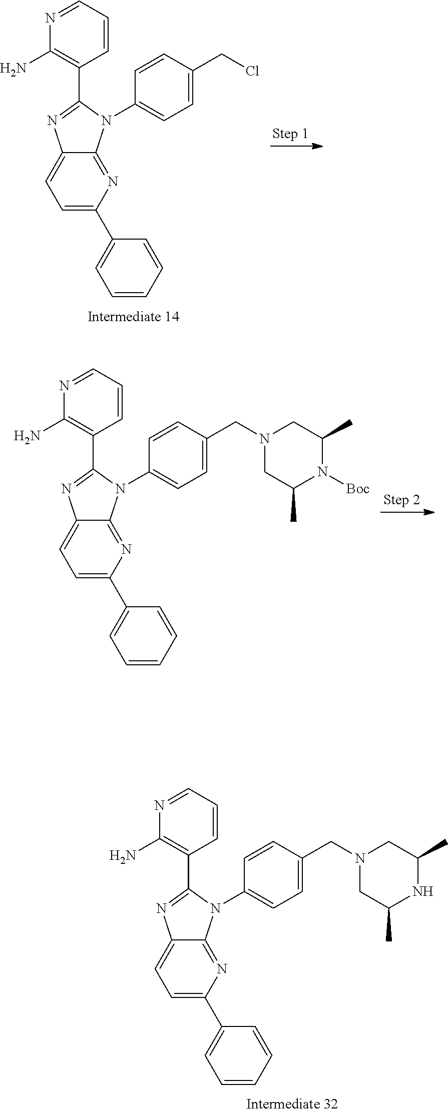 [SMILES: Nc1ncccc1-c1nc2ccc(-c3ccccc3)nc2n1-c1ccc(CCl)cc1 ; C[C@@H]1CN(Cc2ccc(-n3c(-c4cccnc4N)nc4ccc(-c5ccccc5)nc43)cc2)C[C@H](C)N1C(=O)OC(C)(C)C ; C[C@@H]1CN(Cc2ccc(-n3c(-c4cccnc4N)nc4ccc(-c5ccccc5)nc43)cc2)C[C@H](C)N1]

### Step 1: (2S,6R)-tert-Butyl 4-(4-(2-(2-aminopyridin-3-yl)-5-phenyl-3H-imidazo[4,5-b]pyridin-3-yl)benzyl)-2,6-dimethylpiperazine-1-carboxylate

To a solution of Intermediate 14 (1.0 g, 2.43 mmol) and tert-butyl (2S,6R)-2,6-dimethylpiperazine-1-carboxylate (520 mg, 2.43 mmol) in DMF (10 mL) was added K₂CO₃ (671 mg, 4.86 mmol). The mixture was stirred at 25° C. for 16 hr. The reaction mixture was diluted with H₂O (10 mL) at 25° C. and extracted with EtOAc (10 mL×3). The combined organic layers were washed with brine (10 mL×3), dried over Na₂SO₄, filtered, and concentrated under reduced pressure. The residue was purified by silica gel flash chromatography (Eluent of 1~2% MeOH in CH₂Cl₂) to give (2S,6R)-tert-butyl 4-(4-(2-(2-aminopyridin-3-yl)-5-phenyl-3H-imidazo[4,5-b]pyridin-3-yl)benzyl)-2,6-dimethylpiperazine-1-carboxylate (770 mg, yield: 54%) as a yellow solid, which was used directly in the next step. MS: m/z=590.4 [M+H]⁺.

### Step 2: 3-(3-(4-(((3S,5R)-3,5-Dimethylpiperazin-1-yl)methyl)phenyl)-5-phenyl-3H-imidazo[4,5-b]pyridin-2-yl)pyridin-2-amine

To a solution of tert-butyl (2S,6R)-4-[[4-[2-(2-amino-3-pyridyl)-5-phenyl-imidazo[4,5-b]pyridin-3-yl]phenyl]methyl]-2,6-dimethyl-piperazine-1-carboxylate (50 mg, 84.9 μmol) in 1,4-dioxane (3 mL) was added HCl/1,4-dioxane (4M, 5 mL). The mixture was stirred at 25° C. for 1 hr. The reaction mixture was concentrated under reduced pressure to remove 1,4-dioxane.

The crude was dissolved in H₂O (5 mL) and extracted with CH₂Cl₂ (5 mL×3). The aqueous phase was added NaHCO₃ and extracted with CH₂Cl₂ (5 mL×3). The combined organic layers were concentrated to give 3-(3-(4-(((3S,5R)-3,5-dimethylpiperazin-1-yl)methyl)phenyl)-5-phenyl-3H-imidazo[4,5-b]pyridin-2-yl)pyridin-2-amine (Intermediate 32, 34 mg, yield: 82%) as a yellow solid. MS: m/z=490.1 [M+H]⁺. ¹H NMR (400 MHz, Dimethylsulfoxide-d₆) δ 8.27 (d, J=8.4 Hz, 1H), 7.97-8.04 (m, 4H), 7.42-7.48 (m, 6H), 7.37-7.42 (m, 1H), 7.15 (dd, J=7.6, 2.0 Hz, 1H), 7.05 (br s, 2H), 6.36 (dd, J=7.6, 4.8 Hz, 1H), 3.52 (s, 2H), 2.73-2.79 (m, 2H), 2.64-2.67 (m, 2H), 1.56-1.50 (m, 2H), 0.91 (d, J=6.0 Hz, 6H).

### Intermediate 33: 3-(3-(4-(((3R,5R)-3,5-Dimethylpiperazin-1-yl)methyl)phenyl)-5-phenyl-3H-imidazo[4,5-b]pyridin-2-yl)pyridin-2-amine

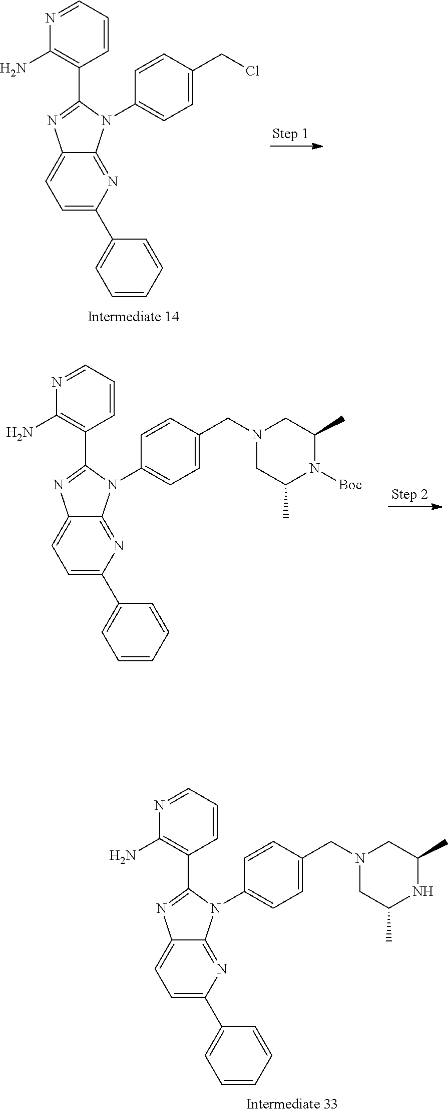 [SMILES: Nc1ncccc1-c1nc2ccc(-c3ccccc3)nc2n1-c1ccc(CCl)cc1 ; C[C@@H]1CN(Cc2ccc(-n3c(-c4cccnc4N)nc4ccc(-c5ccccc5)nc43)cc2)C[C@@H](C)N1C(=O)OC(C)(C)C ; C[C@@H]1CN(Cc2ccc(-n3c(-c4cccnc4N)nc4ccc(-c5ccccc5)nc43)cc2)C[C@@H](C)N1]

### Step 1: tert-Butyl (2R,6R)-4-(4-(2-(2-aminopyridin-3-yl)-5-phenyl-3H-imidazo[4,5-b]pyridin-3-yl)benzyl)-2,6-dimethylpiperazine-1-carboxylate

To a solution of Intermediate 14 (1.06 g, 2.57 mmol) and tert-butyl (2R,6R)-2,6-dimethylpiperazine-1-carboxylate (500 mg, 2.33 mmol) in DMF (10 mL) was added DIEA (905 mg, 7.0 mmol). The mixture was stirred at 80° C. for 16 hr. The mixture was quenched with H₂O (40 mL) and extracted with EtOAc (40 mL×3). The combined organic phase was dried over anhydrous Na₂SO₄, filtered, and concentrated. The residue was purified by silica gel flash chromatography (Eluent of 0~9% MeOH in CH₂Cl₂) to give tert-butyl (2R,6R)-4-(4-(2-(2-aminopyridin-3-yl)-5-phenyl-3H-imidazo[4,5-b]pyridin-3-yl)benzyl)-2,6-dimethylpiperazine-1-carboxylate (Intermediate 27, 1 g, yield: 69.4%) as a yellow solid. MS: m/z=590.4 [M+H]⁺. ¹H NMR (400 MHz, Dimethylsulfoxide-d₆) δ 8.27 (d, J=8.4 Hz, 1H), 8.05-7.98 (m, 4H), 7.52-7.43 (m, 6H), 7.42-7.36 (m, 1H), 7.16 (dd, J=7.6, 1.6 Hz, 1H), 7.04 (br s, 2H), 6.37 (dd, J=7.6, 4.8 Hz, 1H), 3.83-3.74 (m, 2H), 3.65 (d, J=13.6 Hz, 1H), 3.48 (d, J=13.6 Hz, 1H), 3.35-3.39 (m, 2H), 2.27-2.19 (m, 2H), 1.42-1.41 (m, 1H), 1.40 (s, 9H), 1.23 (d, J=6.4 Hz, 6H).

### Step 2: 3-(3-(4-(((3R,5R)-3,5-Dimethylpiperazin-1-yl)methyl)phenyl)-5-phenyl-3H-imidazo[4,5-b]pyridin-2-yl)pyridin-2-amine

A solution of tert-butyl (2R,6R)-4-(4-(2-(2-aminopyridin-3-yl)-5-phenyl-3H-imidazo[4,5-b]pyridin-3-yl)benzyl)-2,6-dimethylpiperazine-1-carboxylate (100 mg, 169 μmol) in HCl/1,4-dioxane (4M, 1 mL) was stirred at 25° C. for 0.5 hr. The mixture was filtered to give 3-(3-(4-(((3R,5R)-3,5-dimethylpiperazin-1-yl)methyl)phenyl)-5-phenyl-3H-imidazo[4,5-b]pyridin-2-yl)pyridin-2-amine (Intermediate 33, 84 mg HCl salt, yield: 95%) as a yellow solid. MS: m/z=490.2 [M+H]⁺. ¹H NMR (400 MHz, Dimethylsulfoxide-d₆) δ 12.41-11.57 (m, 0.5H), 10.79-10.27 (m, 0.5H), 10.07-9.71 (m, 1H), 8.65-8.51 (m, 1H), 8.38 (d, J=8.4 Hz, 1H), 8.16 (d, J=6.0 Hz, 1H), 8.11-7.95 (m, 4H), 7.93-7.82 (m, 3H), 7.68 (d, J=8.0 Hz, 2H), 7.51-7.42 (m, 3H), 7.04 (t, J=6.8 Hz, 1H), 4.50-4.25 (m, 2H), 3.44-3.32 (m, 2H), 3.20-3.01 (m, 2H), 2.91-2.87 (m, 1H), 2.75-2.71 (m, 1H), 1.60-1.31 (m, 6H).

### Intermediate 34: 3-(3-(4-(((3S,5S)-3,5-Dimethylpiperazin-1-yl)methyl)phenyl)-5-phenyl-3H-imidazo[4,5-b]pyridin-2-yl)pyridin-2-amine

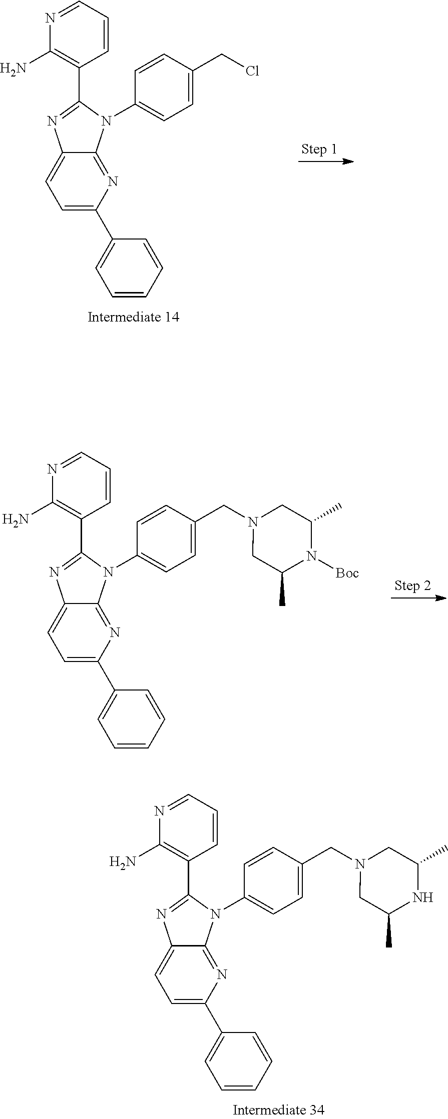 [SMILES: Nc1ncccc1-c1nc2ccc(-c3ccccc3)nc2n1-c1ccc(CCl)cc1 ; C[C@H]1CN(Cc2ccc(-n3c(-c4cccnc4N)nc4ccc(-c5ccccc5)nc43)cc2)C[C@H](C)N1C(=O)OC(C)(C)C ; C[C@H]1CN(Cc2ccc(-n3c(-c4cccnc4N)nc4ccc(-c5ccccc5)nc43)cc2)C[C@H](C)N1]

### Step 1: tert-Butyl (2S,6S)-4-(4-(2-(2-aminopyridin-3-yl)-5-phenyl-3H-imidazo[4,5-b]pyridin-3-yl)benzyl)-2,6-dimethylpiperazine-1-carboxylate

To a solution of Intermediate 14 (1.0 g, 2.43 mmol) in DMF (4 mL) were added K₂CO₃ (671 mg, 4.86 mmol) and tert-butyl (2R,6R)-2,6-dimethylpiperazine-1-carboxylate (624 mg, 2.91 mmol). The mixture was stirred at 25° C. for 16 hr. The reaction mixture was quenched with H₂O (10 mL) at 25° C., diluted with CH₂Cl₂ (6 mL), and washed with H₂O (20 mL×3). The organic layers were washed with brine (20 mL), dried over Na₂SO₄, filtered, and concentrated under reduced pressure. The residue was purified by silica gel flash chromatography (Eluent of 0-10% MeOH in CH₂Cl₂) to give tert-butyl (2S,6S)-4-(4-(2-(2-aminopyridin-3-yl)-5-phenyl-3H-imidazo[4,5-b]pyridin-3-yl)benzyl)-2,6-dimethylpiperazine-1-carboxylate (750 mg, yield: 52%) as a yellow solid. MS: m/z=590.3 [M+H]⁺. ¹H NMR (400 MHz, Dimethylsulfoxide-d₆) δ 8.26 (d, J=8.4 Hz, 1H), 8.04-7.95 (m, 4H), 7.51-7.38 (m, 7H), 7.16 (dd, J=7.6, 1.6 Hz, 1H), 7.04 (br s, 2H), 6.38 (dd, J=8.0, 4.8 Hz, 1H), 3.82-3.75 (m, 2H), 3.64 (d, J=13.6 Hz, 1H), 3.47 (d, J=13.6 Hz, 1H), 2.90-2.86 (m, 1H), 2.76-2.70 (m, 1H), 2.26-2.18 (m, 2H), 1.40 (s, 9H), 1.22 (d, J=6.4 Hz, 6H).

### Step 2: 3-(3-(4-(((3S,5S)-3,5-Dimethylpiperazin-1-yl)methyl)phenyl)-5-phenyl-3H-imidazo[4,5-b]pyridin-2-yl)pyridin-2-amine

A solution of tert-butyl (2S,6S)-4-(4-(2-(2-aminopyridin-3-yl)-5-phenyl-3H-imidazo[4,5-b]pyridin-3-yl)benzyl)-2,6-dimethylpiperazine-1-carboxylate (700 mg, 1.19 mmol) in HCl/1,4-dioxane (10 mL) was stirred at 25° C. for 2 hr. The reaction was filtered and concentrated under reduced pressure to give 3-(3-(4-(((3S,5S)-3,5-dimethylpiperazin-1-yl)methyl)phenyl)-5-phenyl-3H-imidazo[4,5-b]pyridin-2-yl)pyridin-2-amine (Intermediate 34, 540 mg, HCl salt, yield: 86%) as a white solid. MS: m/z=490.6 [M+H]⁺. ¹H NMR (400 MHz, Dimethylsulfoxide-d₆) δ 8.27 (d, J=8.4 Hz, 1H), 8.08-7.95 (m, 4H), 7.52-7.35 (m, 7H), 7.15 (dd, J=7.6, 1.6 Hz, 1H), 7.04 (br s, 2H), 6.40-6.34 (m, 1H), 3.54 (d, J=14.0 Hz, 1H), 3.45-3.40 (m, 1H), 3.10-3.04 (m, 2H), 2.41-2.33 (m, 2H), 2.06-2.00 (m, 2H), 1.04 (d, J=6.4 Hz, 6H).

### Intermediate 35: 3-(3-(4-((3,8-diazabicyclo[3.2.1]octan-8-yl)methyl)phenyl)-5-phenyl-3H-imidazo[4,5-b]pyridin-2-yl)pyridin-2-amine

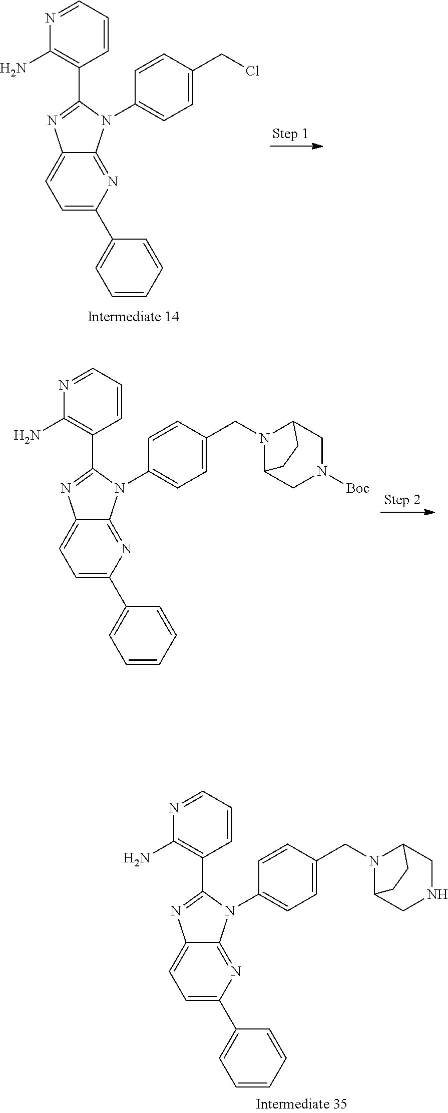 [SMILES: Nc1ncccc1-c1nc2ccc(-c3ccccc3)nc2n1-c1ccc(CCl)cc1 ; CC(C)(C)OC(=O)N1CC2CCC(C1)N2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C3CCC2CNC3)cc1]

### Step 1: tert-Butyl 8-(4-(2-(2-aminopyridin-3-yl)-5-phenyl-3H-imidazo[4,5-b]pyridin-3-yl)benzyl)-3,8-diazabicyclo[3.2.1]octane-3-carboxylate

To a solution of Intermediate 14 (1.0 g, 2.43 mmol) and tert-butyl 3,8-diazabicyclo[3.2.1]octane-3-carboxylate (618 mg, 2.91 mmol) in DMF (15 mL) was added K₂CO₃ (671 mg, 4.86 mmol). The mixture was stirred at 25° C. for 8 hr. The reaction mixture was diluted with H₂O (10 mL) at 25° C. and extracted with CH₂Cl₂ (20 mL×3). The combined organic layers were washed with brine (30 ml), dried over Na₂SO₄, filtered, and concentrated under reduced pressure. The residue was purified by silica gel flash chromatography (Eluent of 0-10% EtOAc in petroleum ether) to give tert-butyl 8-(4-(2-(2-aminopyridin-3-yl)-5-phenyl-3H-imidazo[4,5-b]pyridin-3-yl)benzyl)-3,8-diazabicyclo[3.2.1]octane-3-carboxylate (956 mg, yield: 67%) as a yellow solid. MS: m/z=588.2 [M+H]⁺. ¹H NMR (400 MHz, Dimethylsulfoxide-d₆) δ 8.26 (d, J=8.4 Hz, 1H), 8.05-7.95 (m, 4H), 7.55 (d, J=8.4 Hz, 2H), 7.48-7.37 (m, 5H), 7.16 (dd, J=7.6, 1.6 Hz, 1H), 7.01 (br s, 2H), 6.38 (dd, J=7.6, 4.8 Hz, 1H), 3.59 (s, 2H), 3.20-2.85 (m, 6H), 2.02-1.92 (m, 2H), 1.54-1.48 (m, 2H), 1.40 (s, 9H).

### Step 2: 3-(3-(4-((3,8-Diazabicyclo[3.2.1]octan-8-yl)methyl)phenyl)-5-phenyl-3H-imidazo[4,5-b]pyridin-2-yl)pyridin-2-amine

To a solution of tert-butyl 8-(4-(2-(2-aminopyridin-3-yl)-5-phenyl-3H-imidazo[4,5-b]pyridin-3-yl)benzyl)-3,8-diazabicyclo[3.2.1]octane-3-carboxylate (600 mg, 1.02 mmol) in 1,4-dioxane (5 mL) was added HCl/1,4-dioxane (4M, 5 mL) at 25° C. The mixture was stirred at 25° C. for 6 hr. The reaction was filtered and concentrated under reduced pressure to give 3-(3-(4-((3,8-diazabicyclo[3.2.1]octan-8-yl)methyl)phenyl)-5-phenyl-3H-imidazo[4,5-b]pyridin-2-yl)pyridin-2-amine (Intermediate 35, 535 mg, HCl salt) as a light yellow solid. MS: m/z=488.2 [M+H]⁺. ¹H NMR (400 MHz, Dimethylsulfoxide-d₆) δ 12.42 (br s, 1H), 10.4-10.0 (m, 2H), 8.55 (br s, 1H), 8.38 (d, J=8.0 Hz, 1H), 8.13-8.07 (m, 1H), 8.06-8.00 (m, 5H), 7.98-7.94 (m, 1H), 7.69 (d, J=8.0 Hz, 2H), 7.50-7.40 (m, 3H), 6.94 (t, J=7.2 Hz, 1H), 4.39-4.33 (m, 2H), 4.01 (s, 2H), 3.95-3.89 (m, 3H), 3.42-3.39 (m, 3H), 2.47-2.38 (m, 2H).

### Intermediate 36: 3-(3-(4-((3,8-diazabicyclo[3.2.1]octan-3-yl)methyl)phenyl)-5-phenyl-3H-imidazo[4,5-b]pyridin-2-yl)pyridin-2-amine

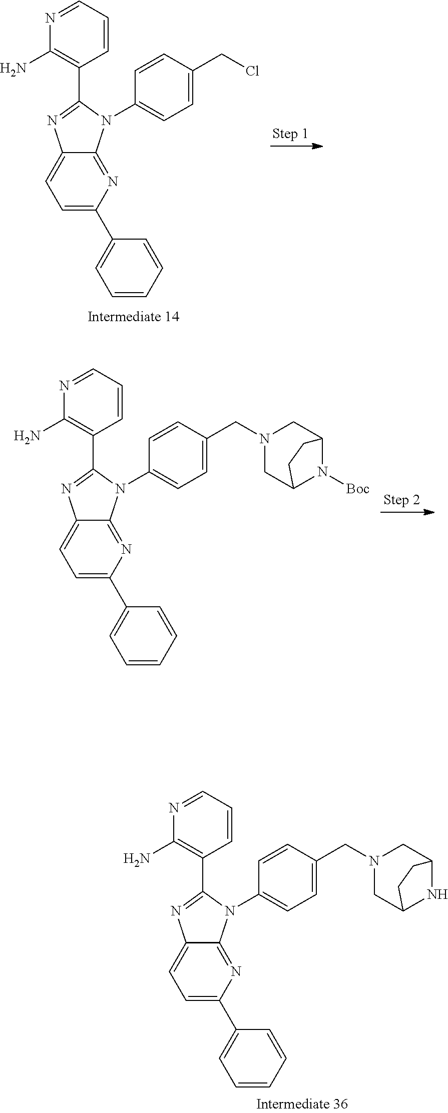 [SMILES: Nc1ncccc1-c1nc2ccc(-c3ccccc3)nc2n1-c1ccc(CCl)cc1 ; CC(C)(C)OC(=O)N1C2CCC1CN(Cc1ccc(-n3c(-c4cccnc4N)nc4ccc(-c5ccccc5)nc43)cc1)C2 ; Nc1ncccc1-c1nc2ccc(-c3ccccc3)nc2n1-c1ccc(CN2CC3CCC(C2)N3)cc1]

### Step 1: tert-Butyl 3-(4-(2-(2-aminopyridin-3-yl)-5-phenyl-3H-imidazo[4,5-b]pyridin-3-yl)benzyl)-3,8-diazabicyclo[3.2.1]octane-8-carboxylate

To a solution of Intermediate 14 (500 mg, 1.21 mmol) and tert-butyl 3,8-diazabicyclo[3.2.1]octane-8-carboxylate (309 mg, 1.46 mmol) in DMF (8 mL) was added K₂CO₃ (336 mg, 2.43 mmol). The mixture was stirred at 25° C. for 8 hr. The reaction mixture was diluted with H₂O (10 mL) at 25° C. and extracted with CH₂Cl₂ (20 mL×3). The combined organic layers were washed with brine (30 mL), dried over Na₂SO₄, filtered, and concentrated under reduced pressure. The residue was purified by silica gel flash chromatography (Eluent of 0-10% EtOAc in petroleum ether) to give tert-butyl 3-(4-(2-(2-aminopyridin-3-yl)-5-phenyl-3H-imidazo[4,5-b]pyridin-3-yl)benzyl)-3,8-diazabicyclo[3.2.1]octane-8-carboxylate (450 mg, yield: 63%) as a yellow solid. MS: m/z=588.3 [M+H]⁺.

### Step 2: 3-(3-(4-((3,8-Diazabicyclo[3.2.1]octan-3-yl)methyl)phenyl)-5-phenyl-3H-imidazo[4,5-b]pyridin-2-yl)pyridin-2-amine

To a solution of tert-butyl 3-(4-(2-(2-aminopyridin-3-yl)-5-phenyl-3H-imidazo[4,5-b]pyridin-3-yl)benzyl)-3,8-diazabicyclo[3.2.1]octane-8-carboxylate (450 mg, 0.766 mmol) in 1,4-dioxane (5 mL) was added HCl/1,4-dioxane (4M, 5 mL) at 25° C. The mixture was stirred at 25° C. for 6 hr. The reaction was filtered and concentrated under reduced pressure to give 3-(3-(4-((3,8-diazabicyclo[3.2.1]octan-3-yl)methyl)phenyl)-5-phenyl-3H-imidazo[4,5-b]pyridin-2-yl)pyridin-2-amine (Intermediate 36, 240 mg, HCl salt) as a light yellow solid. MS: m/z=488.1 [M+H]⁺. ¹H NMR (400 MHz, Methanol-d₄) δ 8.34-8.28 (m, 1H), 8.05-7.95 (m, 4H), 7.90-7.85 (m, 3H), 7.65 (d, J=8.0 Hz, 2H), 7.48-7.37 (m, 3H), 6.90 (t, J=6.8 Hz, 1H), 4.35-4.25 (m, 4H), 3.50-3.40 (m, 4H), 2.41 (d, J=8.4 Hz, 2H), 2.25-2.16 (m, 2H).

### Intermediate 37: 1-(4-(2-(2-aminopyridin-3-yl)-5-phenyl-3H-imidazo[4,5-b]pyridin-3-yl)benzyl)azepan-4-amine

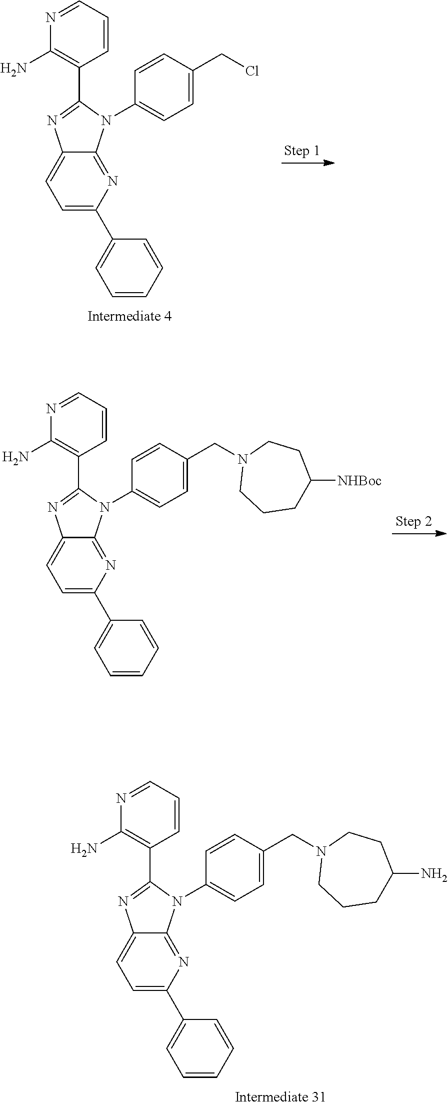 [SMILES: Nc1ncccc1-c1nc2ccc(-c3ccccc3)nc2n1-c1ccc(CCl)cc1 ; CC(C)(C)OC(=O)NC1CCCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCCC(N)CC2)cc1]

### Step 1: tert-Butyl (1-(4-(2-(2-aminopyridin-3-yl)-5-phenyl-3H-imidazo[4,5-b]pyridin-3-yl)benzyl)azepan-4-yl)carbamate

To a solution of Intermediate 14 (1.0 g, 2.43 mmol) and tert-butyl N-(azepan-4-yl)carbamate (670 mg, 2.67 mmol) in DMF (10 mL) was added DIEA (1.26 g, 9.71 mmol). The mixture was stirred at 80° C. for 16 hr. The mixture was quenched with H₂O (40 mL) and extracted with EtOAc (40 mL×3). The combined organic phase was dried over anhydrous Na₂SO₄, filtered and concentrated. The residue was purified by silica gel flash chromatography (Eluent of 0~10% MeOH in CH₂Cl₂) to give tert-butyl (1-(4-(2-(2-aminopyridin-3-yl)-5-phenyl-3H-imidazo[4,5-b]pyridin-3-yl)benzyl)azepan-4-yl)carbamate (890 mg, yield: 62%) as a yellow solid. MS: m/z=590.4 [M+H]⁺. ¹H NMR (400 MHz, Chloroform-d) δ 8.13 (d, J=8.4 Hz, 1H), 8.09-8.05 (m, 1H), 8.02 (d, J=7.6 Hz, 2H), 7.80 (d, J=8.4 Hz, 1H), 7.55-7.47 (m, 2H), 7.47-7.33 (m, 5H), 7.15-7.06 (m, 1H), 6.61 (br s, 2H), 6.35 (dd, J=7.6, 4.8 Hz, 1H), 5.08-4.93 (m, 1H), 3.99-3.83 (m, 1H), 3.73 (s, 2H), 2.88-2.68 (m, 2H), 2.67-2.52 (m, 2H), 2.00-1.68 (m, 6H), 1.43 (s, 9H).

### Step 2: 1-(4-(2-(2-Aminopyridin-3-yl)-5-phenyl-3H-imidazo[4,5-b]pyridin-3-yl)benzyl)azepan-4-amine

A solution of tert-butyl (1-(4-(2-(2-aminopyridin-3-yl)-5-phenyl-3H-imidazo[4,5-b]pyridin-3-yl)benzyl)azepan-4-yl)carbamate (100 mg, 170 μmol) in HCl in 1,4-dioxane (4M, 1 mL) was stirred at 25° C. for 1 hr. The mixture was filtered to give 1-(4-(2-(2-aminopyridin-3-yl)-5-phenyl-3H-imidazo[4,5-b]pyridin-3-yl)benzyl)azepan-4-amine (Intermediate 37, 80 mg HCl salt, yield: 89%) as a yellow solid. MS: m/z=490.2 [M+H]⁺. ¹H NMR (400 MHz, Dimethylsulfoxide-d₆) δ 11.56-11.29 (m, 1H), 8.37 (J=8.4 Hz, 1H), 8.36-8.18 (m, 3H), 8.14 (J=7.6 Hz, 1H), 8.12-7.97 (m, 3H), 7.96-7.80 (m, 3H), 7.67 (d, J=8.4 Hz, 2H), 7.52-7.38 (m, 3H), 6.93-6.84 (m, 1H), 4.52-4.33 (m, 2H), 3.40-3.38 (m, 2H), 3.21-3.12 (m, 2H), 3.09-2.96 (m, 1H), 2.26-1.98 (m, 4H), 1.93-1.80 (m, 1H), 1.75-1.61 (m, 1H).

### Intermediate 38: 1-(4-(2-(2-aminopyridin-3-yl)-5-phenyl-3H-imidazo[4,5-b]pyridin-3-yl)benzyl)azepan-3-amine

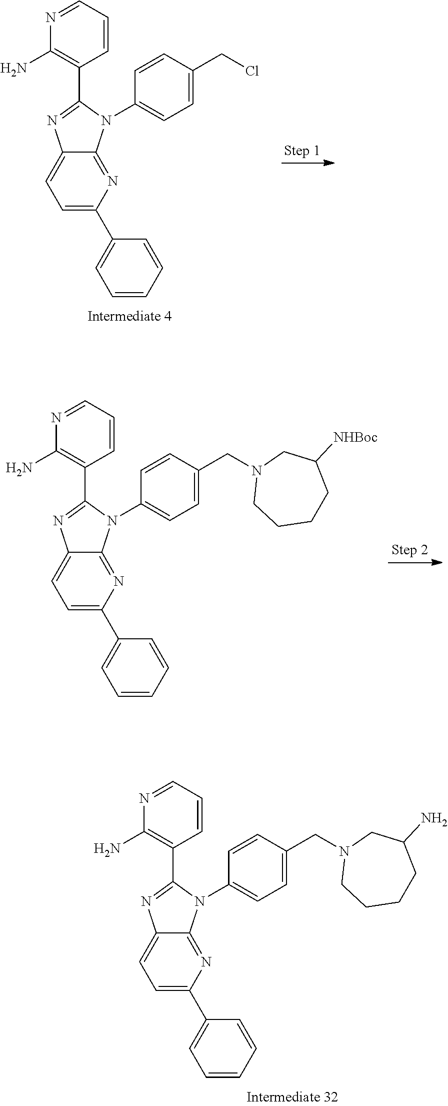 [SMILES: Nc1ncccc1-c1nc2ccc(-c3ccccc3)nc2n1-c1ccc(CCl)cc1 ; CC(C)(C)OC(=O)NC1CCCCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CCCCC(N)C2)cc1]

### Step 1: tert-Butyl (1-(4-(2-(2-aminopyridin-3-yl)-5-phenyl-3H-imidazo[4,5-b]pyridin-3-yl)benzyl)azepan-3-yl)carbamate

To a solution of Intermediate 14 (800 mg, 1.94 mmol) and tert-butyl N-(azepan-3-yl)carbamate (416 mg, 1.94 mmol) in MeCN (10 mL) were added K₂CO₃ (805 mg, 5.83 mmol) and NaI (58.2 mg, 388 μmol). The mixture was stirred at 80° C. for 16 hr. The reaction mixture was diluted with H₂O (50 mL) and extracted with EtOAc (30 mL×3). The combined organic layers were washed with brine (10 mL×5), dried over Na₂SO₄, filtered, and concentrated under reduced pressure. The residue was purified by silica gel flash chromatography (Eluent of 2~3% MeOH in CH₂Cl₂) to give tert-butyl (1-(4-(2-(2-aminopyridin-3-yl)-5-phenyl-3H-imidazo[4,5-b]pyridin-3-yl)benzyl)azepan-3-yl)carbamate (600 mg, yield: 52%) as yellow solid. MS: m/z=590.4 [M+H]⁺. ¹H NMR (400 MHz, Dimethylsulfoxide-d₆) δ 8.27 (d, J=8.4 Hz, 1H), 8.03 (d, J=7.6 Hz, 2H), 8.00-7.96 (m, 2H), 7.51 (d, J=8.4 Hz, 2H), 7.49-7.36 (m, 6H), 7.12 (dd, J=7.6, 1.6 Hz, 1H), 7.08 (br s, 1H), 6.65 (d, J=8.4 Hz, 1H), 6.37 (dd, J=7.6, 4.8 Hz, 1H), 3.74 (s, 2H), 3.60-3.53 (m, 1H), 2.82-2.77 (m, 1H), 2.66-2.61 (m, 1H), 2.56-2.52 (m, 2H), 1.80-1.72 (m, 1H), 1.66-1.61 (m, 2H), 1.56-1.46 (m, 3H), 1.33 (s, 9H).

### Step 2: 1-(4-(2-(2-Aminopyridin-3-yl)-5-phenyl-3H-imidazo[4,5-b]pyridin-3-yl)benzyl)azepan-3-amine

A solution of tert-butyl (1-(4-(2-(2-aminopyridin-3-yl)-5-phenyl-3H-imidazo[4,5-b]pyridin-3-yl)benzyl)azepan-3-yl)carbamate (50 mg, 170 μmol) in HCl in 1,4-dioxane (4M, 1 mL) was stirred at 25° C. for 1 hr. The mixture was filtered to give 1-(4-(2-(2-aminopyridin-3-yl)-5-phenyl-3H-imidazo[4,5-b]pyridin-3-yl)benzyl)azepan-3-amine (Intermediate 38, 83 mg, HCl salt yield: 93%) as a yellow solid. MS: m/z=490.2 [M+H]⁺. ¹H NMR (400 MHz, Dimethylsulfoxide-d₆) δ 8.27 (d, J=8.4 Hz, 1H), 8.05-7.98 (m, 4H), 7.53-7.37 (m, 7H), 7.16-7.12 (m, 1H), 7.07 (br s, 2H), 6.38 (dd, J=7.6, 4.8 Hz, 1H), 3.77-3.72 (m, 2H), 3.62-3.53 (m, 1H), 2.89-2.78 (m, 2H), 2.65-2.61 (m, 2H), 2.59-2.53 (m, 2H), 1.82-1.73 (m, 1H), 1.67-1.48 (m, 4H), 1.46-1.25 (m, 1H).

### Intermediate 39: 3-(3-(4-(((1S,4S)-2,5-Diazabicyclo[2.2.1]heptan-2-yl)methyl)phenyl)-5-phenyl-3H-imidazo[4,5-b]pyridin-2-yl)pyridin-2-amine

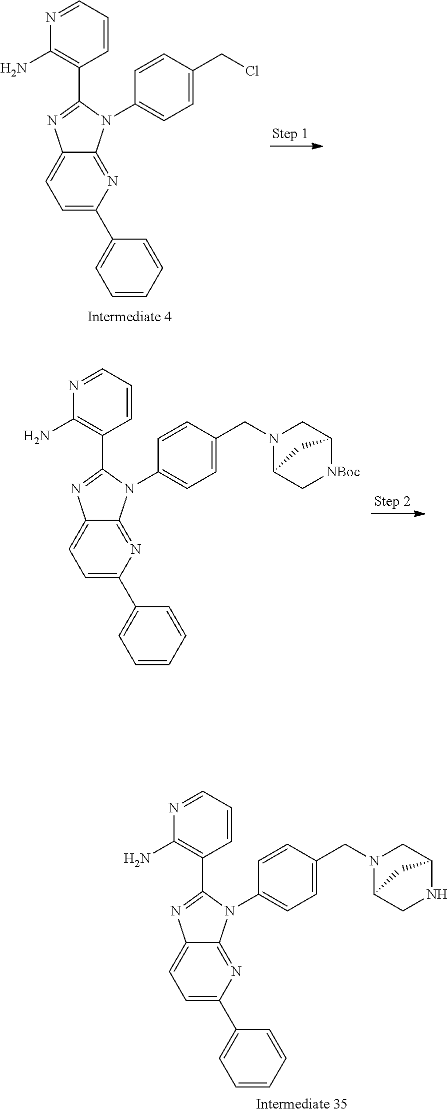 [SMILES: Nc1ncccc1-c1nc2ccc(-c3ccccc3)nc2n1-c1ccc(CCl)cc1 ; CC(C)(C)OC(=O)N1C[C@@H]2C[C@H]1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C[C@@H]3C[C@H]2CN3)cc1]

### Step 1: tert-Butyl (1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate

To a solution of Intermediate 14 (1.0 g, 2.43 mmol) in DMF (10 mL) were added DIEA (1.27 mL, 7.28 mmol) and tert-butyl (1S,4S)-2,5-diazabicyclo[2.2.1]heptane-2-carboxylate (530 mg, 2.67 mmol). The mixture was degassed and purged with N₂ three times and stirred at 80° C. for 16 hr under N₂. The reaction mixture was diluted with CH₂Cl₂(80 mL) at 25° C., washed with brine (50 mL×3), dried over Na₂SO₄, filtered, and concentrated under reduced pressure. The residue was purified by silica gel flash chromatography (Eluent of 1-4% MeOH in CH₂Cl₂) to give tert-butyl (1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate (1.14 g, yield: 75%) as a yellow solid. MS: m/z=574.4 [M+H]⁺. ¹H NMR (400 MHz, Chloroform-d) δ 8.12 (d, J=8.4 Hz, 1H), 8.06 (dd, J=4.8, 1.6 Hz, 1H), 8.04-7.98 (m, 2H), 7.80 (d, J=8.4 Hz, 1H), 7.50 (d, J=8.4 Hz, 2H), 7.47-7.33 (m, 5H), 7.10 (d, J=7.6 Hz, 1H), 6.60 (br s, 2H), 6.36 (dd, J=8.0, 4.8 Hz, 1H), 4.47-4.24 (m, 1H), 3.88-3.80 (m, 2H), 3.71-3.49 (m, 2H), 3.28-3.16 (m, 1H), 3.02-2.89 (m, 1H), 2.82-2.57 (m, 1H), 1.96-1.87 (m, 1H), 1.77-1.68 (m, 1H), 1.48 (s, 9H).

### Step 2: 3-(3-(4-(((1S,4S)-2,5-Diazabicyclo[2.2.1]heptan-2-yl)methyl)phenyl)-5-phenyl-3H-imidazo[4,5-b]pyridin-2-yl)pyridin-2-amine

To a solution of tert-butyl (1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate (50 mg, 87.2 μmol) in dioxane (0.5 mL) was added HCl in 1,4-dioxane (4M, 1 mL). The mixture was degassed and purged with N₂ three times and stirred at 25° C. for 2 hr under N₂. The reaction mixture was concentrated under reduced pressure. The residue was triturated with 1,4-dioxane (1 mL) at 25° C. for 10 min. 3-(3-(4-(((1S,4S)-2,5-Diazabicyclo[2.2.1]heptan-2-yl)methyl)phenyl)-5-phenyl-3H-imidazo[4,5-b]pyridin-2-yl)pyridin-2-amine (Intermediate 39, 41 mg, HCl salt, yield: 95%) was obtained as a light-yellow solid. MS: m/z=474.2 [M+H]⁺. ¹H NMR (400 MHz, Dimethylsulfoxide-d₆) δ 12.43-11.57 (m, 1H), 10.39-9.51 (m, 2H), 8.55-8.39 (m, 1H), 8.38 (d, J=8.4 Hz, 1H), 8.14 (d, J=5.2 Hz, 1H), 8.11-8.01 (m, 3H), 7.94 (d, J=8.0 Hz, 2H), 7.84 (d, J=7.2 Hz, 1H), 7.69 (d, J=8.4 Hz, 2H), 7.54-7.40 (m, 3H), 6.91-6.87 (m, 1H), 4.68-4.40 (m, 4H), 3.99-3.94 (m, 1H), 3.80-3.75 (m, 2H), 3.42-3.40 (m, 1H), 2.60-2.52 (m, 1H), 2.20-2.06 (m, 1H).

### Intermediate 40: 3-(3-(4-(((1R,4R)-2,5-Diazabicyclo[2.2.1]heptan-2-yl)methyl)phenyl)-5-phenyl-3H-imidazo[4,5-b]pyridin-2-yl)pyridin-2-amine

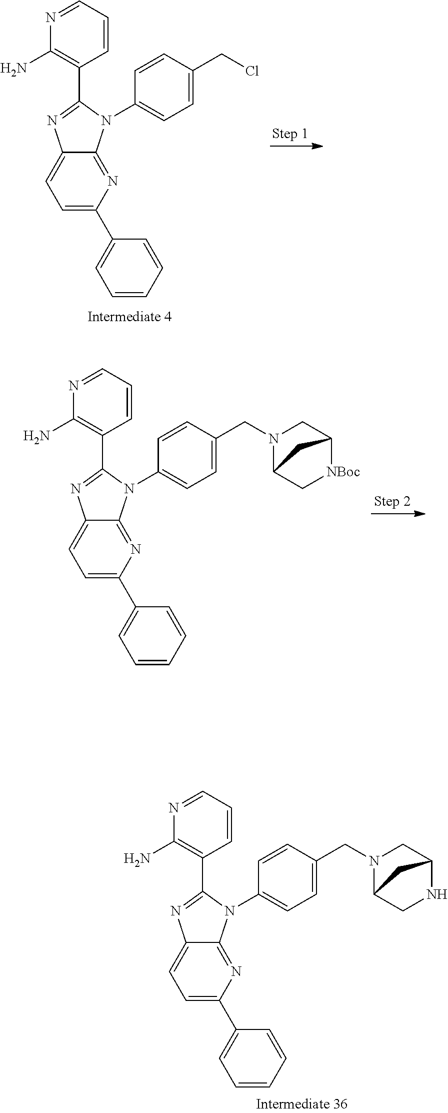 [SMILES: Nc1ncccc1-c1nc2ccc(-c3ccccc3)nc2n1-c1ccc(CCl)cc1 ; CC(C)(C)OC(=O)N1C[C@H]2C[C@@H]1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C[C@H]3C[C@@H]2CN3)cc1]

### Step 1: tert-Butyl (1R,4R)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate

To a solution of Intermediate 14 (1.0 g, 2.43 mmol) and tert-butyl (1R,4R)-2,5-diazabicyclo[2.2.1]heptane-2-carboxylate (578 mg, 2.91 mmol) in DMF (15 mL) was added K₂CO₃ (671 mg, 4.86 mmol). The mixture was stirred at 25° C. for 16 hr. The reaction mixture was quenched with H₂O (10 ml) at 25° C. and extracted with EtOAc (50 mL×3). The combined organic layers were washed with brine (20 mL×3), dried over Na₂SO₄, filtered, and concentrated under reduced pressure. The residue was purified by silica gel flash chromatography ((Eluent of 0-10% MeOH in CH₂Cl₂) to give tert-butyl (1R,4R)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate (1.0 g, yield: 72%) as a yellow solid. MS: m/z=574.2 [M+H]⁺.

### Step 2: 3-(3-(4-(((1R,4R)-2,5-Diazabicyclo[2.2.1]heptan-2-yl)methyl)phenyl)-5-phenyl-3H-imidazo[4,5-b]pyridin-2-yl)pyridin-2-amine

To a solution of tert-butyl (1R,4R)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate (1.0 g, 1.74 mmol) in 1,4-dioxane (2 mL) was added HCl/1,4-dioxane (4M, 10 mL). The mixture was stirred at 25° C. for 2 hr. The reaction mixture was concentrated under reduced pressure to remove 1,4-dioxane. 3-(3-(4-(((1R,4R)-2,5-Diazabicyclo[2.2.1]heptan-2-yl)methyl)phenyl)-5-phenyl-3H-imidazo[4,5-b]pyridin-2-yl)pyridin-2-amine (Intermediate 40, 854 mg, 2HCl salt, yield: 90%) was obtained as a yellow solid. MS: m/z=474.2 [M+H]⁺. ¹H NMR (400 MHz, Methanol-d₄) δ 8.33 (d, J=8.4 Hz, 1H), 8.06-8.00 (m, 6H), 7.88 (dd, J=7.6, 1.6 Hz, 1H), 7.73 (d, J=8.4 Hz, 2H), 7.46-7.40 (m, 3H), 6.92 (dd, J=7.6, 6.4 Hz, 1H), 4.84-4.81 (m, 1H), 4.75-4.65 (m, 2H), 4.14 (d, J=12.8 Hz, 1H), 3.96-3.92 (m, 1H), 3.74-3.71 (m, 1H), 3.62-3.60 (m, 1H), 3.38-3.32 (m, 1H), 2.87-2.82 (m, 1H), 2.39-2.36 (m, 1H).

### Intermediate 41: 3-(5-Phenyl-3-(4-((piperidin-4-ylamino)methyl)phenyl)-3H-imidazo[4,5-b]pyridin-2-yl)pyridin-2-amine

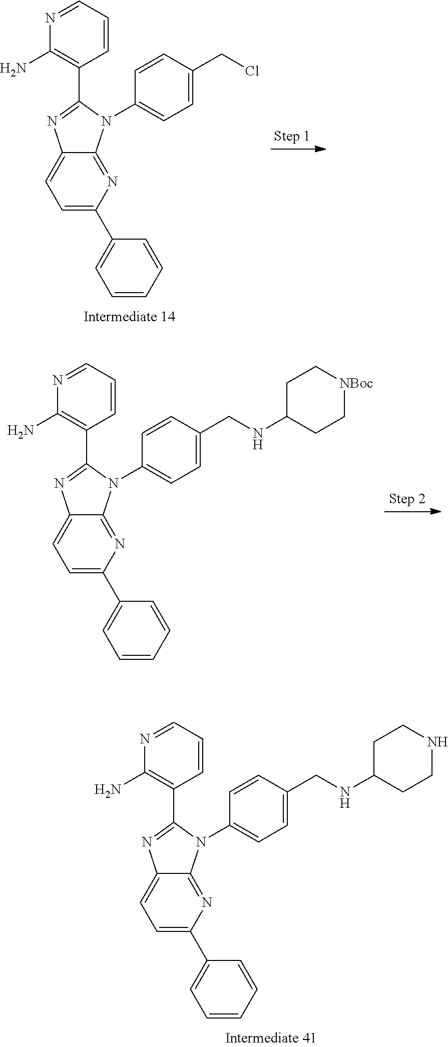 [SMILES: Nc1ncccc1-c1nc2ccc(-c3ccccc3)nc2n1-c1ccc(CCl)cc1 ; CC(C)(C)OC(=O)N1CCC(NCc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CNC2CCNCC2)cc1]

### Step 1: tert-Butyl 4-((4-(2-(2-Aminopyridin-3-yl)-5-phenyl-3H-imidazo[4,5-b]pyridin-3-yl)benzyl)amino)piperidine-1-carboxylate

To a solution of Intermediate 14 (200 mg, 486 μmol) and tert-butyl 4-aminopiperidine-1-carboxylate (117 mg, 583 μmol) in DMF (3 mL) were added NaI (7.28 mg, 48.6 μmol) and K₂CO₃ (134 mg, 971 mol). The mixture was stirred at 80° C. for 2 hr. The reaction mixture was poured into H₂O (20 mL). The resulting mixture was extracted with EtOAc (20 mL×2). The combined organic layers were washed with brine (30 mL), dried over Na₂SO₄, filtered, and concentrated under reduced pressure. After purified by silica gel flash chromatography (MeOH in CH₂Cl₂=0% to 10%), tert-butyl 4-((4-(2-(2-aminopyridin-3-yl)-5-phenyl-3H-imidazo[4,5-b]pyridin-3-yl)benzyl)amino)piperidine-1-carboxylate (90 mg, yield: 26%) was obtained as a light-yellow solid, which was directly used in the next step. MS: m/z=576.4 [M+H]⁺.

### Step 2: 3-(5-Phenyl-3-(4-((piperidin-4-ylamino)methyl)phenyl)-3H-imidazo[4,5-b]pyridin-2-yl)pyridin-2-amine

A solution of tert-butyl 4-((4-(2-(2-aminopyridin-3-yl)-5-phenyl-3H-imidazo[4,5-b]pyridin-3-yl)benzyl)amino)piperidine-1-carboxylate (90 mg, 156 μmol) in HCl in 1,4-dioxane (4M, 2 mL) was stirred at 25° C. for 1 hr. The reaction mixture was concentrated to give 3-(5-phenyl-3-(4-((piperidin-4-ylamino)methyl)phenyl)-3H-imidazo[4,5-b]pyridin-2-yl)pyridin-2-amine (Intermediate 41, 80 mg HCl salt, yield: 100%) as a light-yellow oil. MS: m/z=476.2 [M+H]⁺.

### Intermediate 42: 3-(5-Phenyl-3-(4-((piperidin-4-yloxy)methyl)phenyl)-3H-imidazo[4,5-b]pyridin-2-yl)pyridin-2-amine

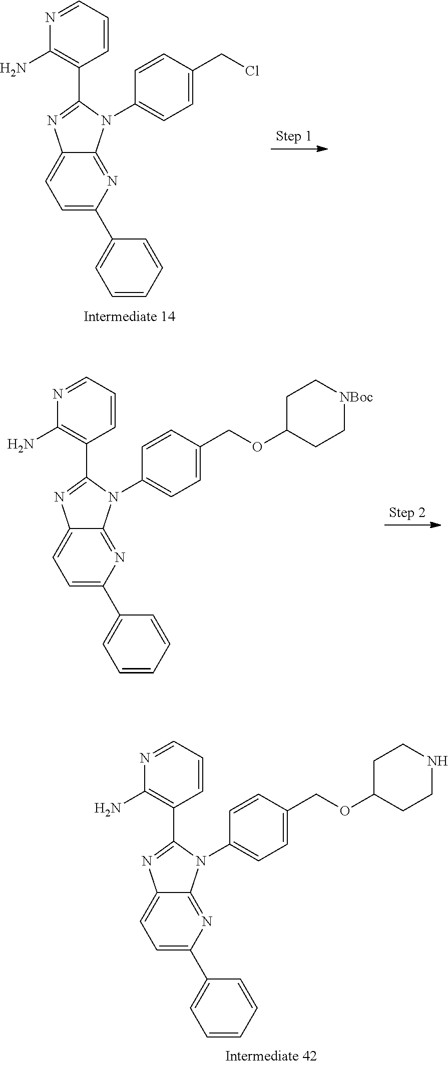 [SMILES: Nc1ncccc1-c1nc2ccc(-c3ccccc3)nc2n1-c1ccc(CCl)cc1 ; CC(C)(C)OC(=O)N1CCC(OCc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(COC2CCNCC2)cc1]

### Step 1: tert-Butyl 4-((4-(2-(2-aminopyridin-3-yl)-5-phenyl-3H-imidazo[4,5-b]pyridin-3-yl)benzyl)oxy)piperidine-1-carboxylate

To a solution of tert-butyl 4-hydroxypiperidine-1-carboxylate (220 mg, 1.1 mmol) in THF (10 mL) at 0° C. was added NaH (58 mg, 1.5 mmol, 60% purity). After addition, the mixture was stirred at 0° C. for 15 min. Intermediate 14 (300 mg, 728 gmol) was added. The resulting mixture was stirred at 65° C. for 16 hr. The reaction mixture was quenched with H₂O (20 mL) at 0° C. and extracted with EtOAc (20 mL×2). The combined organic layers were washed with brine (10 mL), dried over Na₂SO₄, filtered, and concentrated under reduced pressure. After purified by silica gel flash chromatography (MeOH in CH₂Cl₂=0% to 7%), tert-butyl4-((4-(2-(2-aminopyridin-3-yl)-5-phenyl-3H-imidazo[4,5-b]pyridin-3-yl)benzyl)oxy)piperidine-1-carboxylate (350 mg, yield: 83%) was obtained as a yellow solid. ¹H NMR (400 MHz, Chloroform-d) δ 8.13 (d, J=8.4 Hz, 1H), 8.03-8.00 (m, 2H), 7.81 (d, J=8.4 Hz, 1H), 7.56 (d, J=8.4 Hz, 2H), 7.48-7.37 (m, 6H), 7.11 (dd, J=8.0, 2.0 Hz, 1H), 6.67 (br s, 2H), 6.41 (dd, J=7.6, 4.8 Hz, 1H), 4.68 (s, 2H), 3.88-3.87 (m, 1H), 3.04-3.00 (m, 4H), 1.87-1.84 (m, 4H), 1.45 (s, 9H).

### Step 2: 3-(5-Phenyl-3-(4-((piperidin-4-yloxy)methyl)phenyl)-3H-imidazo[4,5-b]pyridin-2-yl)pyridin-2-amine

A solution of tert-butyl4-((4-(2-(2-aminopyridin-3-yl)-5-phenyl-3H-imidazo[4,5-b]pyridin-3-yl)benzyl)oxy)piperidine-1-carboxylate (280 mg, 485 μmol) in 4 M HC in 1,4-dioxane (5 mL) was stirred at 25° C. for 2 hr. The reaction mixture was concentrated under reduced pressure to give 3-(5-phenyl-3-(4-((piperidin-4-yloxy)methyl)phenyl)-3H-imidazo[4,5-b]pyridin-2-yl)pyridin-2-amine (Intermediate 42, 200 mg, HCl salt) as a light-yellow solid, which was used in the next step without further purification. MS: m/z=477.1 [M+H]⁺.

### Intermediate 43: (R)-(4-(4-(2-(2-Aminopyridin-3-yl)-5-phenyl-3H-imidazo[4,5-b]pyridin-3-yl)benzyl)piperazin-2-yl)methanol

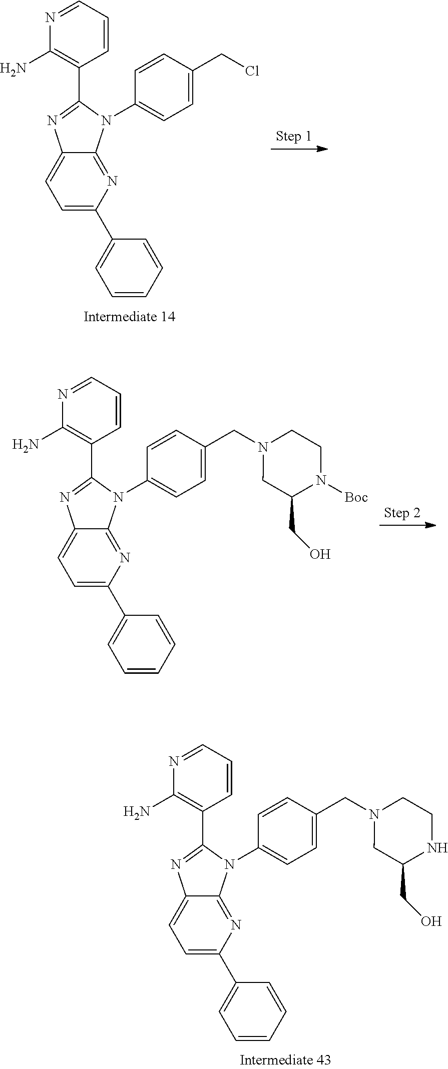 [SMILES: Nc1ncccc1-c1nc2ccc(-c3ccccc3)nc2n1-c1ccc(CCl)cc1 ; CC(C)(C)OC(=O)N1CCN(Cc2ccc(-n3c(-c4cccnc4N)nc4ccc(-c5ccccc5)nc43)cc2)C[C@@H]1CO ; Nc1ncccc1-c1nc2ccc(-c3ccccc3)nc2n1-c1ccc(CN2CCN[C@@H](CO)C2)cc1]

### Step 1: tert-Butyl (R)-4-(4-(2-(2-aminopyridin-3-yl)-5-phenyl-3H-imidazo[4,5-b]pyridin-3-yl)benzyl)-2-(hydroxymethyl)piperazine-1-carboxylate

To a solution of Intermediate 14 (100 mg, 242 μmol) in DMF (5 mL) were added K₂CO₃ (100 mg, 728 mol), NaI (10.9 mg, 72.8 mol) and tert-butyl (R)-2-(hydroxymethyl)piperazine-1-carboxylate (53 mg, 242 μmol). The mixture was stirred at 25° C. for 16 hr. The reaction mixture was diluted with H₂O (10 mL) at 25° C. and extracted with CH₂Cl₂ (20 mL×3). The combined organic layers were washed with brine (20 mL×3), dried over Na₂SO₄, filtered, and concentrated under reduced pressure. After purified by prep-HPLC (column: Phenomenex C18 150×25 mm×10 μm; mobile phase: [water (NH₄HCO₃)-ACN]; B %: 46%-76%, 14 min), tert-butyl (R)-4-(4-(2-(2-aminopyridin-3-yl)-5-phenyl-3H-imidazo[4,5-b]pyridin-3-yl)benzyl)-2-(hydroxymethyl)piperazine-1-carboxylate (120 mg, yield: 84%) was obtained as a yellow solid. MS: m/z=592.3 [M+H]⁺.

### Step 2: (R)-(4-(4-(2-(2-Aminopyridin-3-yl)-5-phenyl-3H-imidazo[4,5-b]pyridin-3-yl)benzyl)piperazin-2-yl)methanol

To a solution of tert-butyl (R)-4-(4-(2-(2-aminopyridin-3-yl)-5-phenyl-3H-imidazo[4,5-b]pyridin-3-yl)benzyl)-2-(hydroxymethyl)piperazine-1-carboxylate (40 mg, 67.6 μmol) in 1,4-dioxane (2 mL) was added HCl/1,4-dioxane (4 M). The mixture was stirred at 25° C. for 6 hr. The mixture was filtered and concentrated under reduced pressure. (R)-(4-(4-(2-(2-aminopyridin-3-yl)-5-phenyl-3H-imidazo[4,5-b]pyridin-3-yl)benzyl)piperazin-2-yl)methanol (Intermediate 43, 16 mg, yield: 48%) was obtained as a yellow solid. MS: m/z=492.3 [M+H]⁺. ¹H NMR (400 MHz, Methanol-d₄) δ 8.33-8.30 (m, 1H), 8.05-7.98 (m, 41), 7.95-7.85 (m, 3H), 7.72 (m, 2H), 7.48-7.40 (m, 3H), 6.95-6.85 (m, 1H), 4.61 (s, 2H), 3.94-3.68 (m, 7H), 3.50-3.38 (m, 2H).

### Intermediate 44: (S)-(4-(4-(2-(2-Aminopyridin-3-yl)-5-phenyl-3H-imidazo[4,5-b]pyridin-3-yl)benzyl)piperazin-2-yl)methanol

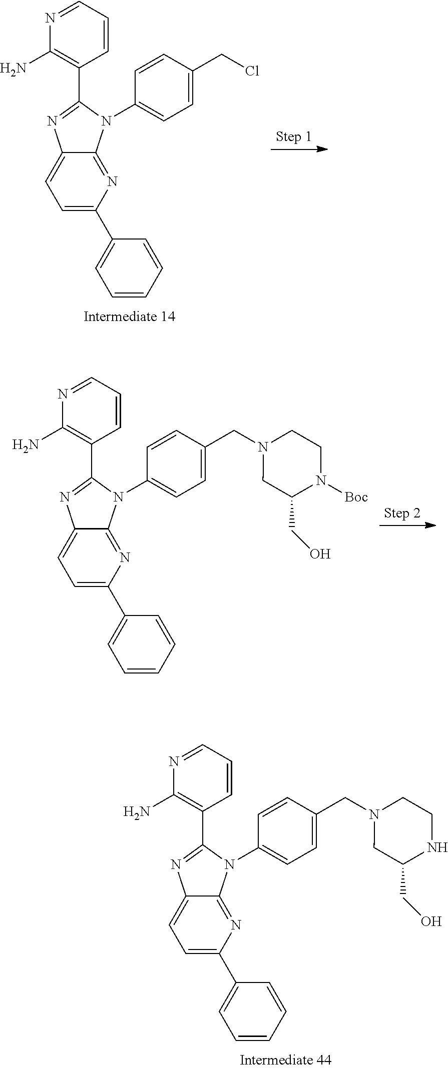 [SMILES: Nc1ncccc1-c1nc2ccc(-c3ccccc3)nc2n1-c1ccc(CCl)cc1 ; CC(C)(C)OC(=O)N1CCN(Cc2ccc(-n3c(-c4cccnc4N)nc4ccc(-c5ccccc5)nc43)cc2)C[C@H]1CO ; Nc1ncccc1-c1nc2ccc(-c3ccccc3)nc2n1-c1ccc(CN2CCN[C@H](CO)C2)cc1]

### Step 1: tert-Butyl (S)-4-(4-(2-(2-aminopyridin-3-yl)-5-phenyl-3H-imidazo[4,5-b]pyridin-3-yl)benzyl)-2-(hydroxymethyl)piperazine-1-carboxylate

To a solution of Intermediate 14 (300 mg, 728 μmol) in DMF (5 mL) were added K₂CO₃ (302 mg, 2.19 mmol), NaI (32.8 mg, 219 μmol), and tert-butyl (S)-2-(hydroxymethyl)piperazine-1-carboxylate (173 mg, 801 mol). The mixture was stirred at 25° C. for 16 hr. The reaction mixture was diluted with H₂O (10 mL) at 25° C. and extracted with CH₂Cl₂ (20 mL×3). The combined organic layers were washed with brine (20 mL×3), dried over Na₂SO₄, filtered, and concentrated under reduced pressure. The crude was purified by silica gel flash chromatography (EtOAc in petroleum ether=20~70%) to give tert-butyl (S)-4-(4-(2-(2-aminopyridin-3-yl)-5-phenyl-3H-imidazo[4,5-b]pyridin-3-yl)benzyl)-2-(hydroxymethyl)piperazine-1-carboxylate (200 mg, yield: 47%) as a yellow solid. MS: m/z=592.3 [M+H]⁺.

### Step 2: (S)-(4-(4-(2-(2-Aminopyridin-3-yl)-5-phenyl-3H-imidazo[4,5-b]pyridin-3-yl)benzyl)piperazin-2-yl)methanol

A solution of tert-butyl (S)-4-(4-(2-(2-aminopyridin-3-yl)-5-phenyl-3H-imidazo[4,5-b]pyridin-3-yl)benzyl)-2-(hydroxymethyl)piperazine-1-carboxylate (200 mg, 338 μmol) in 4 M HCl in 1,4-dioxane (2 mL) was stirred at 25° C. for 2 hr. The reaction was concentrated under reduced pressure to give a yellow solid (250 mg, HCl salt). The solid (50 mg, HCl salt) was diluted with aqueous NaHCO₃ (10 mL) and extracted with CH₂Cl₂ (10 mL×3). The combined organic layers were washed with brine (20 mL), dried over Na₂SO₄, filtered, and concentrated under reduced pressure. After purified by prep-TLC (MeOH in CH₂Cl₂=10%), (S)-(4-(4-(2-(2-aminopyridin-3-yl)-5-phenyl-3H-imidazo[4,5-b]pyridin-3-yl)benzyl)piperazin-2-yl)methanol (Intermediate 44, 20.6 mg, yield: 40%) was obtained as a yellow solid. MS: m/z=492.2 [M+H]⁺. ¹H NMR (400 MHz, Methanol-d₄) δ 8.32 (d, J=8.4 Hz, 1H), 8.06-8.00 (m, 4H), 7.91 (d, J=8.0 Hz, 2H), 7.86 (dd, J=7.6, 1.2 Hz, 1H), 7.72 (d, J=8.0 Hz, 2H), 7.47-7.40 (m, 3H), 6.94-6.86 (m, 1H), 3.92-3.86 (m, 2H), 3.81-3.64 (m, 5H), 3.60 (s, 2H), 3.43-3.34 (m, 2H).

### Intermediate 45: (S)-3-(3-(4-((3-(methylamino)pyrrolidin-1-yl)methyl)phenyl)-5-phenyl-3H-imidazo[4,5-b]pyridin-2-yl)pyridin-2-amine

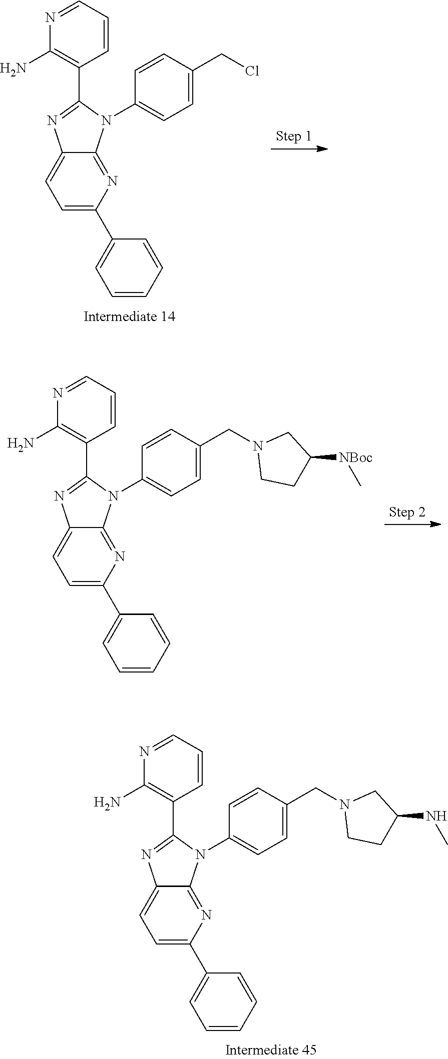 [SMILES: Nc1ncccc1-c1nc2ccc(-c3ccccc3)nc2n1-c1ccc(CCl)cc1 ; CN(C(=O)OC(C)(C)C)[C@H]1CCN(Cc2ccc(-n3c(-c4cccnc4N)nc4ccc(-c5ccccc5)nc43)cc2)C1 ; CN[C@H]1CCN(Cc2ccc(-n3c(-c4cccnc4N)nc4ccc(-c5ccccc5)nc43)cc2)C1]

### Step 1: tert-Butyl (S)-(1-(4-(2-(2-aminopyridin-3-yl)-5-phenyl-3H-imidazo[4,5-b]pyridin-3-yl)benzyl)pyrrolidin-3-yl)(methyl)carbamate

To a solution of Intermediate 14 (200 mg, 486 μmol) in CH₃CN (5 mL) were added tert-butyl (S)-methyl(pyrrolidin-3-yl)carbamate (97 mg, 486 μmol), NaI (7.28 mg, 48.6 μmol) and K₂CO₃ (268 mg, 1.94 mmol). The mixture was stirred at 80° C. for 2 hr. The mixture was concentrated under reduced pressure to give tert-butyl (S)-(1-(4-(2-(2-aminopyridin-3-yl)-5-phenyl-3H-imidazo[4,5-b]pyridin-3-yl)benzyl)pyrrolidin-3-yl)(methyl)carbamate (180 mg, yield: 60%) as a yellow solid. MS: m/z=576.4 [M+H]⁺.

### Step 2: (S)-3-(3-(4-((3-(Methylamino)pyrrolidin-1-yl)methyl)phenyl)-5-phenyl-3H-imidazo[4,5-b]pyridin-2-yl)pyridin-2-amine

To a solution of tert-butyl (S)-(1-(4-(2-(2-aminopyridin-3-yl)-5-phenyl-3H-imidazo[4,5-b]pyridin-3-yl)benzyl)pyrrolidin-3-yl)(methyl)carbamate (250 mg, 433 μmol) in CH₂Cl₂ (5 mL) was added TFA (247 mg, 2.17 mmol). The mixture was stirred at 25° C. for 2 hr. The mixture was diluted with H₂O (5 mL). The pH of the mixture was adjusted to about 8 with NaHCO₃ (aq) and the mixture was extracted with CH₂Cl₂ (10 mL×2). The combined organic layers were washed with brine (10 mL×2), dried over Na₂SO₄, filtered and concentrated under reduced pressure. The mixture was purified by prep-TLC (CH₂Cl₂:MeOH=10:1) to give (S)-3-(3-(4-((3-(methylamino)pyrrolidin-1-yl)methyl)phenyl)-5-phenyl-3H-imidazo[4,5-b]pyridin-2-yl)pyridin-2-amine (Intermediate 45, 200 mg, TFA salt, yield: 81%) as a light-yellow solid. MS: m/z=476.3 [M+H]⁺. ¹H NMR (400 MHz, Methanol-d₄) δ 8.19 (d, J=8.8 Hz, 1H), 8.04-8.00 (m, 2H), 7.98 (dd, J=5.2, 1.6 Hz, 1H), 7.94 (d, J=8.4 Hz, 1H), 7.56 (d, J=8.0 Hz, 2H), 7.47-7.40 (m, 4H), 7.38 (d, J=7.2 Hz, 1H), 7.33 (dd, J=8.0, 1.6 Hz, 1H), 6.48 (dd, J=7.6, 4.8 Hz, 1H), 3.84-3.72 (m, 2H), 3.70-3.64 (m, 1H), 3.01-2.98 (m, 1H), 2.90-2.84 (m, 1H), 2.64 (s, 3H), 2.50-2.44 (m, 1H), 2.38-2.28 (m, 1H), 2.23-1.96 (m, 1H), 1.94-1.83 (m, 1H).

### Intermediate 46: 4-(4-(2-(2-Aminopyridin-3-yl)-5-phenyl-3H-imidazo[4,5-b]pyridin-3-yl)benzyl)piperazin-1-amine

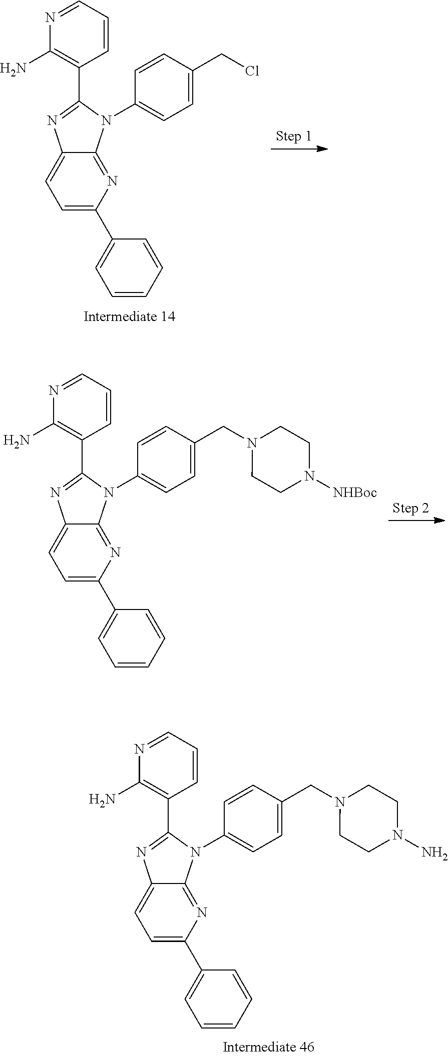 [SMILES: Nc1ncccc1-c1nc2ccc(-c3ccccc3)nc2n1-c1ccc(CCl)cc1 ; CC(C)(C)OC(=O)NN1CCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCN(N)CC2)cc1]

### Step 1: tert-Butyl (4-(4-(2-(2-aminopyridin-3-yl)-5-phenyl-3H-imidazo[4,5-b]pyridin-3-yl)benzyl)piperazin-1-yl)carbamate

To a solution of Intermediate 14 (300 mg, 728 μmol) and tert-butyl piperazin-1-ylcarbamate (146 mg, 728 μmol) in DMF (3 mL) were added K₂CO₃ (302 mg, 2.19 mmol) and NaI (32.7 mg, 218 μmol). The mixture was stirred at 25° C. for 16 hr. The reaction mixture was quenched with H₂O (10 mL) at 25° C., diluted with more H₂O (30 mL) and extracted with CH₂Cl₂ (30 mL×2). The combined organic layers were washed with brine (25 mL×2), dried over Na₂SO₄, filtered, and concentrated under reduced pressure. The crude was purified by silica gel flash chromatography (MeOH in DCM=0% to 10%) to give tert-butyl (4-(4-(2-(2-aminopyridin-3-yl)-5-phenyl-3H-imidazo[4,5-b]pyridin-3-yl)benzyl)piperazin-1-yl)carbamate (184 mg, yield: 61%) as a yellow solid. MS: m/z=577.2 [M+H]⁺.

### Step 2: 4-(4-(2-(2-Aminopyridin-3-yl)-5-phenyl-3H-imidazo[4,5-b]pyridin-3-yl)benzyl)piperazin-1-amine

To a solution of tert-butyl (4-(4-(2-(2-aminopyridin-3-yl)-5-phenyl-3H-imidazo[4,5-b]pyridin-3-yl)benzyl)piperazin-1-yl)carbamate (184 mg, 319 μmol) in 1,4-dioxane (1 mL) was added HCl/1,4-dioxane (4 M, 5 mL). The mixture was stirred at 25° C. for 2 hr. The reaction mixture was filtered and concentrated under reduced pressure. 4-(4-(2-(2-Aminopyridin-3-yl)-5-phenyl-3H-imidazo[4,5-b]pyridin-3-yl)benzyl)piperazin-1-amine (Intermediate 46, 150 mg HCl salt, yield: 98%) was obtained as a yellow solid. MS: m/z=477.2 [M+H]⁺. ¹H NMR (400 MHz, Methanol-d₆) δ 8.33 (d, J=8.4 Hz, 1H), 8.04 (d, J=8.4 Hz, 4H), 7.94-7.86 (m, 3H), 7.74 (d, J=7.2 Hz, 2H), 7.48-7.39 (m, 3H), 6.94-6.87 (m, 1H), 4.56 (s, 2H), 3.77-3.68 (m, 2H), 3.54-3.47 (m, 4H), 3.28-3.21 (m, 2H).

### Intermediate 48: 3-(3-(4-(((3aR,6aS)-Hexahydropyrrolo[3,4-c]pyrrol-2(1H)-yl)methyl)phenyl)-5-phenyl-3H-imidazo[4,5-b]pyridin-2-yl)pyridin-2-amine

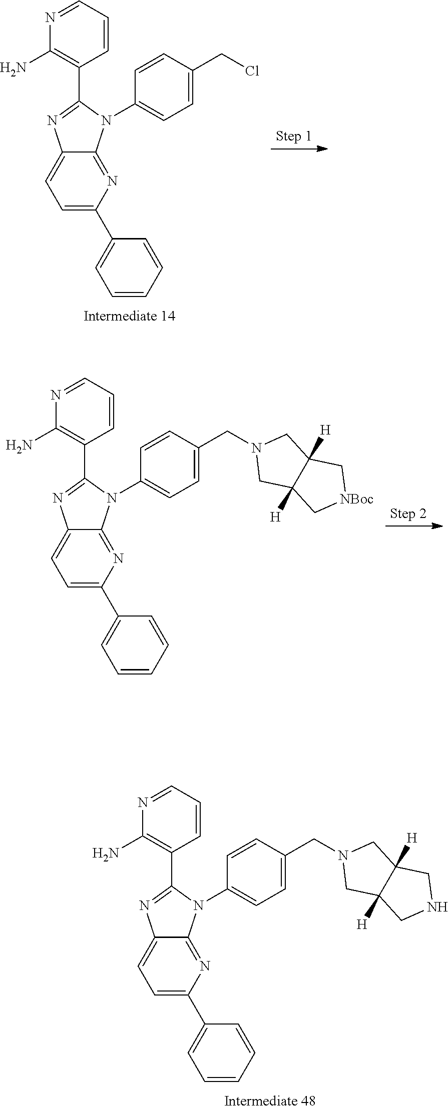 [SMILES: Nc1ncccc1-c1nc2ccc(-c3ccccc3)nc2n1-c1ccc(CCl)cc1 ; CC(C)(C)OC(=O)N1C[C@H]2CN(Cc3ccc(-n4c(-c5cccnc5N)nc5ccc(-c6ccccc6)nc54)cc3)C[C@H]2C1 ; Nc1ncccc1-c1nc2ccc(-c3ccccc3)nc2n1-c1ccc(CN2C[C@H]3CNC[C@H]3C2)cc1]

### Step 1: tert-Butyl (3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrole-2(1H)-carboxylate

To a solution of Intermediate 14 (4.6 g, 11.2 mmol) and tert-butyl (3aR,6aS)-2,3,3a,4,6,6a-hexahydro-1H-pyrrolo[3,4-c]pyrrole-5-carboxylate (2.61 g, 12.3 mmol) in DMF (60 mL) were added NaI (502 mg, 3.35 mmol) and K₂CO₃ (3.09 g, 22.3 mmol) in one portion at 25° C. The mixture was stirred at 80° C. for 12 hr. The reaction mixture was poured into water (100 mL) at 25° C. and extracted with EtOAc (100 mL×3). The combined organic layers were washed with brine (150 mL), dried over anhydrous Na₂SO₄, filtered, and concentrated under reduced pressure. The residue was purified by silica gel flash chromatography (Eluent of 0-6% MeOH in CH₂Cl₂) to give tert-butyl (3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrole-2(1H)-carboxylate (3.6 g, yield: 55%) as a yellow solid. MS: m/z=588.4 [M+H]⁺. ¹H NMR (400 MHz, Dimethylsulfoxide-d₆) δ 8.27 (d, J=8.4 Hz, 1H), 8.06-7.96 (m, 4H), 7.50-7.36 (m, 7H), 7.16 (dd, J=7.6, 1.6 Hz, 1H), 7.03 (br s, 2H), 6.38 (dd, J=7.6, 4.8 Hz, 1H), 3.65 (s, 2H), 3.51-3.41 (m, 2H), 3.17-3.10 (m, 2H), 2.76-2.75 (m, 2H), 2.59-2.53 (m, 2H), 2.45-2.39 (m, 2H), 1.39 (s, 9H).

### Step 2: 3-(3-(4-(((3aR,6aS)-hexahydropyrrolo[3,4-c]pyrrol-2(1H)-yl)methyl)phenyl)-5-phenyl-3H-imidazo[4,5-b]pyridin-2-yl)pyridin-2-amine

To a solution of tert-butyl (3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrole-2(1H)-carboxylate (50 mg, 85.1 μmol) in 1,4-dioxane (2 mL) was added HCl/1,4-dioxane (4 M, 212 L). The reaction mixture was stirred at 20° C. for 12 hr. The mixture was quenched with saturation NaHCO₃ (10 mL) and extracted with CH₂Cl₂ (10 mL×2). The combined organic layers were washed with brine (10 mL), dried over anhydrous Na₂SO₄, filtered and concentrated. 3-(3-(4-(((3aR,6aS)-Hexahydropyrrolo[3,4-c]pyrrol-2(1H)-yl)methyl)phenyl)-5-phenyl-3H-imidazo[4,5-b]pyridin-2-yl)pyridin-2-amine (Intermediate 48, 35.4 mg, yield: 85%) was obtained as an off-white solid. MS: m/z=488.2 [M+H]⁺. ¹H NMR (400 MHz, Dimethylsulfoxide-d₆) 8.27 (d, J=8.0 Hz, 1H), 8.04-7.97 (m, 4H), 7.49-7.44 (m, 5H), 7.43-7.37 (m, 2H), 7.16 (dd, J=7.6, 1.6 Hz, 1H), 7.04 (br s, 2H), 6.38 (dd, J=7.6, 4.8 Hz, 1H), 3.60 (s, 2H), 2.85-2.76 (m, 2H), 2.58-2.51 (m, 7H), 2.31-2.26 (m, 2H).

### Intermediate 49: 3-(3-(4-(1-(4-(Methylamino)piperidin-1-yl)ethyl)phenyl)-5-phenyl-3H-imidazo[4,5-b]pyridin-2-yl)pyridin-2-amine

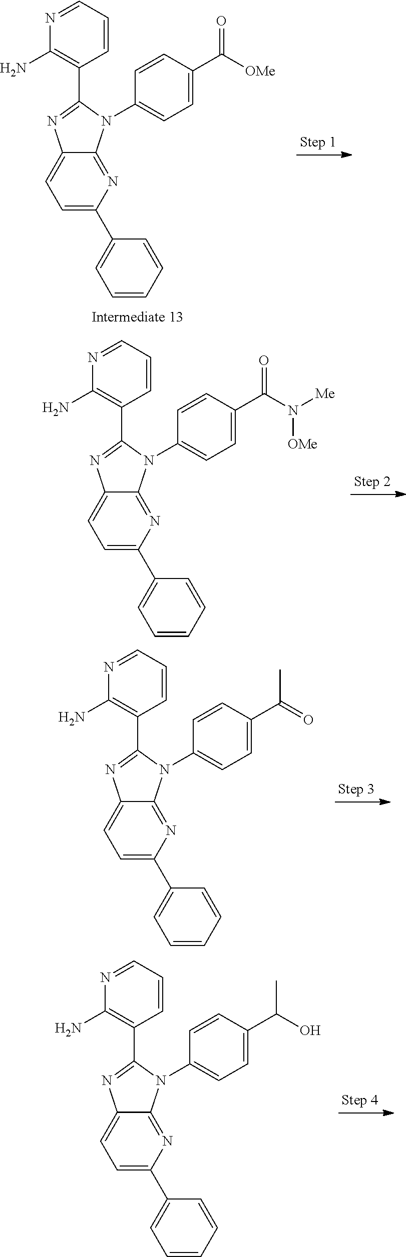

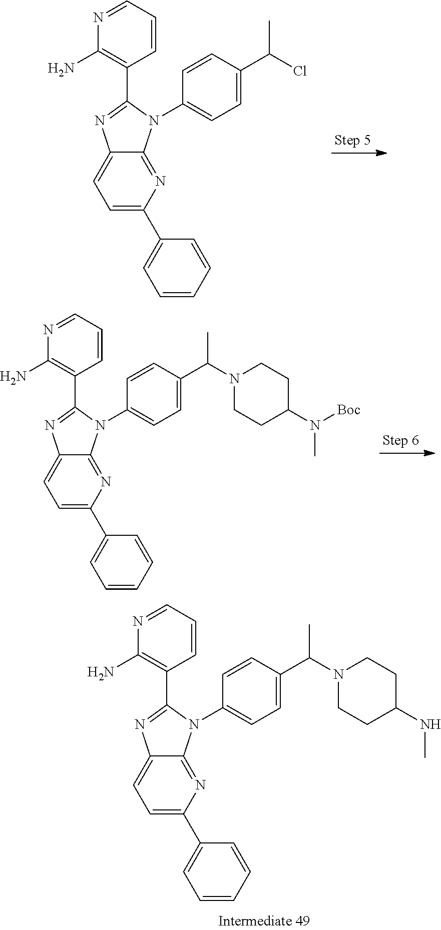

### Step 1: 4-(2-(2-Aminopyridin-3-yl)-5-phenyl-3H-imidazo[4,5-b]pyridin-3-yl)-N-methoxy-N-methylbenzamide

To a solution of Intermediate 13 (1 g, 2.37 mmol) in THE (10 mL) were added MeONMe (HCl salt) (463 mg, 4.75 mmol) and i-PrMgBr (2 M, 5.93 mL) at 0° C. The mixture was stirred at 25° C. for 1 hr. The reaction mixture was quenched with saturated solution of NH₄Cl (10 mL) at 25° C. and extracted with CH₂Cl₂ (10 mL×2). The combined organic layers were washed with brine (10 mL), dried over Na₂SO₄, filtered and concentrated. 4-(2-(2-Aminopyridin-3-yl)-5-phenyl-3H-imidazo[4,5-b]pyridin-3-yl)-N-methoxy-N-methylbenzamide (1.03 g, yield: 86%) was obtained as a yellow solid. MS: m/z=451.1.

### Step 2: 1-(4-(2-(2-Aminopyridin-3-yl)-5-phenyl-3H-imidazo[4,5-b]pyridin-3-yl)phenyl)ethan-1-one

To a solution of 4-(2-(2-aminopyridin-3-yl)-5-phenyl-3H-imidazo[4,5-b]pyridin-3-yl)-N-methoxy-N-methylbenzamide (1.03 g, 2.29 mmol) in THE (10 mL) was added MeMgBr (3 M, 4.57 mL) at 0° C. The mixture was stirred at 25° C. for 0.5 hr. The reaction mixture was quenched with saturated solution of NH₄Cl (20 mL) at 25° C. and extracted with CH₂Cl₂ (50 mL×2). The combined organic layers were washed with brine (20 mL), dried over Na₂SO₄, filtered, and concentrated under reduced pressure. 1-(4-(2-(2-Aminopyridin-3-yl)-5-phenyl-3H-imidazo[4,5-b]pyridin-3-yl)phenyl)ethan-1-one (860 mg, yield: 62%) was obtained as a yellow solid. MS: m/z=406.1.

### Step 3: 1-(4-(2-(2-Aminopyridin-3-yl)-5-phenyl-3H-imidazo[4,5-b]pyridin-3-yl)phenyl)ethan-1-ol

To a solution of 1-(4-(2-(2-aminopyridin-3-yl)-5-phenyl-3H-imidazo[4,5-b]pyridin-3-yl)phenyl)ethan-1-one (860 mg, 2.12 mmol) in THE (10 mL) was added LiAlH₄ (161 mg, 4.24 mmol) at 0° C. The mixture was stirred at 25° C. for 1 hr. The reaction mixture was quenched with Na₂SO₄·10H₂O (150 mg) at 0° C., filtered and concentrated under reduced pressure. 1-(4-(2-(2-Aminopyridin-3-yl)-5-phenyl-3H-imidazo[4,5-b]pyridin-3-yl)phenyl)ethan-1-ol (850 mg, yield: 98%) was obtained as a yellow solid. MS: m/z=408.1.

### Step 4: 3-(3-(4-(1-Chloroethyl)phenyl)-5-phenyl-3H-imidazo[4,5-b]pyridin-2-yl)pyridin-2-amine

To a solution of 1-(4-(2-(2-aminopyridin-3-yl)-5-phenyl-3H-imidazo[4,5-b]pyridin-3-yl)phenyl)ethan-1-ol (500 mg, 1.23 mmol) in CH₂Cl₂ (5 mL) was added SOCl₂ (438 mg, 3.68 mmol). The mixture was stirred at 40° C. for 1 hr. The reaction was concentrated under reduced pressure. 3-(3-(4-(1-Chloroethyl)phenyl)-5-phenyl-3H-imidazo[4,5-b]pyridin-2-yl)pyridin-2-amine (506 mg, yield: 97%) was obtained as a brown solid. MS: m/z=426.0 [M+H]⁺.

### Step 5: tert-Butyl (1-(1-(4-(2-(2-aminopyridin-3-yl)-5-phenyl-3H-imidazo[4,5-b]pyridin-3-yl)phenyl)ethyl)piperidin-4-yl)(methyl)carbamate

To a solution of 3-(3-(4-(1-chloroethyl)phenyl)-5-phenyl-3H-imidazo[4,5-b]pyridin-2-yl)pyridin-2-amine (400 mg, 939 μmol) and tert-butyl methyl(piperidin-4-yl)carbamate (262 mg, 1.22 mmol) in DMF (5 mL) and MeCN (5 mL) was added K₂CO₃ (649 mg, 4.7 mmol). The mixture was stirred at 50° C. for 16 hr. The reaction mixture was diluted with H₂O (5 mL) and extracted with CH₂Cl₂ (20 mL×2). The combined organic layers were washed with brine (5 mL), dried over Na₂SO₄, filtered, and concentrated under reduced pressure. tert-Butyl (1-(1-(4-(2-(2-aminopyridin-3-yl)-5-phenyl-3H-imidazo[4,5-b]pyridin-3-yl)phenyl)ethyl)piperidin-4-yl)(methyl)carbamate (500 mg, yield: 88%) was obtained as a brown liquid. MS: m/z=604.3 [M+H]⁺.

### Step 6: 3-(3-(4-(1-(4-(Methylamino)piperidin-1-yl)ethyl)phenyl)-5-phenyl-3H-imidazo[4,5-b]pyridin-2-yl)pyridin-2-amine

To a solution of tert-butyl (1-(1-(4-(2-(2-aminopyridin-3-yl)-5-phenyl-3H-imidazo[4,5-b]pyridin-3-yl)phenyl)ethyl)piperidin-4-yl)(methyl)carbamate (200 mg, 331 μmol) in 1,4-dioxane (2.0 mL) was added 4M HCl in 1,4-dioxane (2.0 mL). The mixture was stirred at 25° C. for 0.5 hr. The pH was adjusted to 8 with saturated NaHCO₃. The mixture was diluted with H₂O (20 mL) and extracted with CH₂Cl₂ (20 mL×2). The combined organic layers were washed with brine (20 mL), dried over Na₂SO₄, filtered, and concentrated under reduced pressure. 3-(3-(4-(1-(4-(Methylamino)piperidin-1-yl)ethyl)phenyl)-5-phenyl-3H-imidazo[4,5-b]pyridin-2-yl)pyridin-2-amine (Intermediate 49, 97.6 mg, yield: 56%) was obtained as a yellow solid. MS: m/z=504.3. [M+H]⁺. ¹H NMR (400 MHz, Dimethylsulfoxide-d₆) δ 8.27 (d, J=8.4 Hz, 1H), 8.05-7.96 (m, 4H), 7.49-7.38 (m, 7H), 7.16-7.04 (m, 3H), 6.32 (dd, J=8.0, 4.8 Hz, 1H), 3.62-3.55 (m, 1H), 2.91-2.79 (m, 1H), 2.76-2.67 (m, 1H), 2.51-2.47 (m, 2H), 2.24 (s, 3H), 2.20-2.12 (m, 1H), 2.01-1.89 (m, 2H), 1.82-1.72 (m, 2H), 1.35 (d, J=6.8 Hz, 3H).

### Intermediate 50: 3-(3-(4-((4-(Ethylamino)piperidin-1-yl)methyl)phenyl)-5-phenyl-3H-imidazo[4,5-b]pyridin-2-yl)pyridin-2-amine

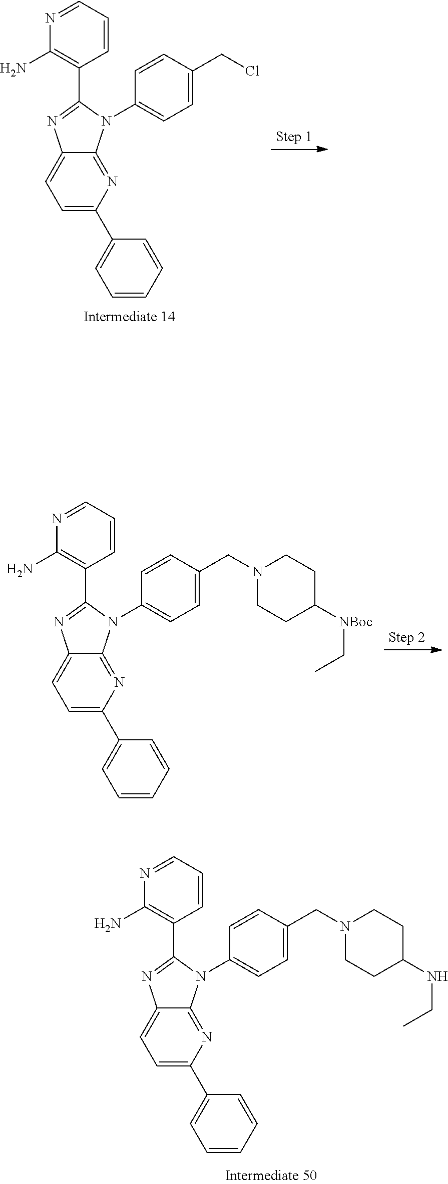 [SMILES: Nc1ncccc1-c1nc2ccc(-c3ccccc3)nc2n1-c1ccc(CCl)cc1 ; CCN(C(=O)OC(C)(C)C)C1CCN(Cc2ccc(-n3c(-c4cccnc4N)nc4ccc(-c5ccccc5)nc43)cc2)CC1 ; CCNC1CCN(Cc2ccc(-n3c(-c4cccnc4N)nc4ccc(-c5ccccc5)nc43)cc2)CC1]

### Step 1: tert-Butyl (1-(4-(2-(2-aminopyridin-3-yl)-5-phenyl-3H-imidazo[4,5-b]pyridin-3-yl)benzyl)piperidin-4-yl)(ethyl)carbamate

To a solution of Intermediate 14 (235 mg, 569 gmol) in DMF (2 mL) were added K₂CO₃ (236 mg, 1.71 mmol), NaI (25.6 mg, 171 gmol) and tert-butyl ethyl(piperidin-4-yl)carbamate (130 mg, 569 μmol). The mixture was stirred at 25° C. for 16 hr. The reaction mixture was poured into H₂O (20 mL). The aqueous phase was extracted with CH₂Cl₂ (20 mL×2). The combined organic layers were washed with brine (20 mL×2), dried over anhydrous Na₂SO₄, filtered and concentrated. tert-Butyl (1-(4-(2-(2-aminopyridin-3-yl)-5-phenyl-3H-imidazo[4,5-b]pyridin-3-yl)benzyl)piperidin-4-yl)(ethyl)carbamate (320 mg, yield: 69%) was obtained as a yellow solid. MS: m/z=604.2 [M+H]⁺.

### Step 2: 3-(3-(4-((4-(Ethylamino)piperidin-1-yl)methyl)phenyl)-5-phenyl-3H-imidazo[4,5-b]pyridin-2-yl)pyridin-2-amine

To a solution of tert-butyl (1-(4-(2-(2-aminopyridin-3-yl)-5-phenyl-3H-imidazo[4,5-b]pyridin-3-yl)benzyl)piperidin-4-yl)(ethyl)carbamate (320 mg, 530 μmol) in 1,4-dioxane (3.0 mL) was added 4M HCl in 1,4-dioxane (3.0 mL). The mixture was stirred at 25° C. for 0.5 hr. The mixture was concentrated to give 3-(3-(4-((4-(ethylamino)piperidin-1-yl)methyl)phenyl)-5-phenyl-3H-imidazo[4,5-b]pyridin-2-yl)pyridin-2-amine (Intermediate 50, 230 mg HCl salt) as a yellow solid. MS: m/z=504.3. [M+H]⁺. ¹H NMR (400 MHz, Dimethylsulfoxide-d₆) δ 8.27 (d, J=8.0 Hz, 1H), 8.06-7.96 (m, 4H), 7.49-7.38 (m, 7H), 7.15 (dd, J=7.6, 1.6 Hz, 1H), 7.03 (br s, 2H), 6.37 (dd, J=7.6, 4.8 Hz, 1H), 3.54 (s, 2H), 2.82-2.73 (m, 2H), 2.58-2.52 (m, 2H), 2.40-2.34 (m, 1H), 2.03-1.93 (m, 2H), 1.82-1.74 (m, 2H), 1.32-1.21 (m, 2H), 0.99 (t, J=7.2 Hz, 3H).

### Intermediate 51: 3-(3-(4-((4-(Cyclopropylamino)piperidin-1-yl)methyl)phenyl)-5-phenyl-3H-imidazo[4,5-b]pyridin-2-yl)pyridin-2-amine

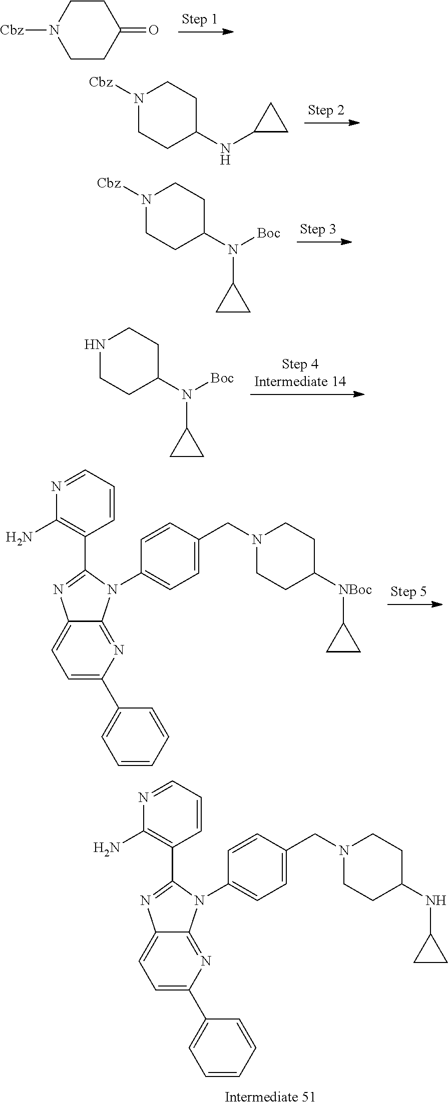

### Step 1: Benzyl 4-(cyclopropylamino)piperidine-1-carboxylate

To a solution of benzyl 4-oxopiperidine-1-carboxylate (500 mg, 2.14 mmol) in CH₂Cl₂ (5 mL) were added cyclopropanamine (184 mg, 3.22 mmol, HCl salt) and AcOH (193 mg, 3.22 mmol). The mixture was stirred at 25° C. for 1 hr and then NaBH(OAc)₃ (681 mg, 3.22 mmol) was added. The resulting mixture was stirred at 25° C. for 16 hr. The reaction mixture was quenched with H₂O (10 mL) at 25° C., diluted with CH₂Cl₂ (10 mL) and extracted with CH₂Cl₂ (15 mL×2). The combined organic layers were washed with brine (15 mL), dried over Na₂SO₄, filtered, and concentrated under reduced pressure. Benzyl 4-(cyclopropylamino)piperidine-1-carboxylate (580 mg, yield: 93%) was obtained as a yellow oil. MS: m/z=275.1 [M+H]⁺. ¹H NMR (400 MHz, Chloroform-d) δ 7.42-7.27 (m, 5H), 5.12 (s, 2H), 4.15-4.03 (m, 2H), 2.96-2.85 (m, 2H), 2.82-2.71 (m, 1H), 2.17-2.09 (m, 1H), 1.97-1.88 (m, 2H), 1.76-1.65 (m, 2H), 1.32-1.23 (m, 2H), 0.48-0.42 (m, 2H), 0.36-0.30 (m, 2H).

### Step 2: Benzyl 4-((tert-butoxycarbonyl)(cyclopropyl)amino)piperidine-1-carboxylate

To a solution of benzyl 4-(cyclopropylamino)piperidine-1-carboxylate (580 mg, 2.11 mmol) in THF (10 mL) and H₂O (5 mL) were added Na₂CO₃ (672 mg, 6.34 mmol) and (Boc)₂O (554 mg, 2.54 mmol). The mixture was stirred at 25° C. for 16 hr. The reaction mixture was quenched with H₂O (5 mL) at 25° C., diluted with CH₂Cl₂ (10 mL) and extracted with CH₂Cl₂ (15 mL×2). The combined organic layers were washed with brine (15 mL), dried over Na₂SO₄, filtered, and concentrated under reduced pressure. Benzyl 4-((tert-butoxycarbonyl)(cyclopropyl)amino)piperidine-1-carboxylate (800 mg, yield: 91%) was obtained as a yellow oil. MS: m/z=397.2 [M+Na]⁺. H NMR (400 MHz, Chloroform-d) δ 7.45-7.27 (m, 5H), 5.12 (s, 2H), 4.35-4.17 (m, 2H), 3.84-3.72 (m, 1H), 2.86-2.71 (m, 2H), 2.36-2.25 (m, 1H), 1.97-1.82 (m, 2H), 1.76-1.66 (m, 2H), 1.52 (s, 9H), 0.79-0.71 (m, 2H), 0.68-0.59 (m, 2H).

### Step 3: tert-Butyl cyclopropyl(piperidin-4-yl)carbamate

To a solution of benzyl 4-((tert-butoxycarbonyl)(cyclopropyl)amino)piperidine-1-carboxylate (500 mg, 1.34 mmol) in MeOH (10 mL) was added Pd/C (150 mg, 10% purity) under N₂ atmosphere. The mixture was purged with H₂ three times and stirred at 40° C. under H₂ atmosphere (40 Psi) for 16 hr. The mixture was filtered and washed with MeOH (10 mL×2).

The filtrate was concentrated to give tert-butyl cyclopropyl(piperidin-4-yl)carbamate (280 mg, yield: 83%) as a white oil. ¹H NMR (400 MHz, Chloroform-d) δ 3.77-3.68 (m, 1H), 3.13-3.07 (m, 2H), 2.67-2.57 (m, 2H), 2.36-2.28 (m, 1H), 1.93-1.82 (m, 2H), 1.74-1.67 (m, 2H), 1.63-1.55 (m, 1H), 1.45 (s, 9H), 0.76-0.71 (m, 2H), 0.68-0.62 (m, 2H).

### Step 4: tert-Butyl (1-(4-(2-(2-aminopyridin-3-yl)-5-phenyl-3H-imidazo[4,5-b]pyridin-3-yl)benzyl)piperidin-4-yl)(cyclopropyl)carbamate

To a solution of Intermediate 14 (300 mg, 728 gmol) in DMF (10 mL) were added K₂CO₃ (302 mg, 2.19 mmol), NaI (10.9 mg, 72.8 μmol) and tert-butyl cyclopropyl(piperidin-4-yl)carbamate (350 mg, 1.46 mmol). The mixture was stirred at 25° C. for 16 hr. The reaction mixture was quenched with H₂O (10 mL) at 25° C., diluted with EtOAc (15 mL) and washed with H₂O (20 mL×3). The combined organic layers were washed with brine (20 mL), dried over Na₂SO₄, filtered, and concentrated under reduced pressure. After purified by silica gel flash chromatography (MeOH in CH₂Cl₂=1% to 5%), tert-butyl (1-(4-(2-(2-aminopyridin-3-yl)-5-phenyl-3H-imidazo[4,5-b]pyridin-3-yl)benzyl)piperidin-4-yl)(cyclopropyl)carbamate (400 mg, yield: 83%) was obtained as a yellow solid. MS: m/z=616.3 [M+H]⁺.

### Step 5: 3-(3-(4-((4-(Cyclopropylamino)piperidin-1-yl)methyl)phenyl)-5-phenyl-3H-imidazo[4,5-b]pyridin-2-yl)pyridin-2-amine

To a solution of tert-butyl (1-(4-(2-(2-aminopyridin-3-yl)-5-phenyl-3H-imidazo[4,5-b]pyridin-3-yl)benzyl)piperidin-4-yl)(cyclopropyl)carbamate (150 mg, 244 μmol) in CH₂Cl₂ (3 mL) was added TFA (27.8 mg, 244 μmol). The mixture was stirred at 25° C. for 16 hr. The pH of the mixture was adjusted to 8 with saturated NaHCO₃. The mixture was diluted with CH₂Cl₂ (15 mL), extracted with CH₂Cl₂ (20 mL×3). The combined organic layers were washed with brine (20 mL), dried over Na₂SO₄, filtered and concentrated under reduced pressure, 3-(3-(4-((4-(Cyclopropylamino)piperidin-1-yl)methyl)phenyl)-5-phenyl-3H-imidazo[4,5-b]pyridin-2-yl)pyridin-2-amine (Intermediate 51, 130 mg, yield: 96%) was obtained as a yellow solid. MS: m/z=516.3 [M+H]⁺. ¹H NMR (400 MHz, Dimethylsulfoxide-d₆) δ 8.27 (d, J=8.4 Hz, 1H), 8.05-7.97 (m, 4H), 7.49-7.42 (m, 6H), 7.41-7.38 (m, 1H), 7.16 (dd, J=7.6, 1.6 Hz, 1H), 7.01 (br s, 2H), 6.38 (dd, J=7.6, 4.8 Hz, 1H), 3.58 (s, 2H), 2.91-2.82 (m, 2H), 2.81-2.71 (m, 1H), 2.13-2.00 (m, 2H), 1.95-1.88 (m, 2H), 1.48-1.37 (m, 2H), 1.29-1.14 (m, 2H), 0.59-0.48 (m, 2H), 0.47-0.34 (m, 2H).

### Intermediate 52: 4-(4-(2-(2-Aminopyridin-3-yl)-5-phenyl-3H-imidazo[4,5-b]pyridin-3-yl)benzyl)-N-methylpiperazin-1-amine

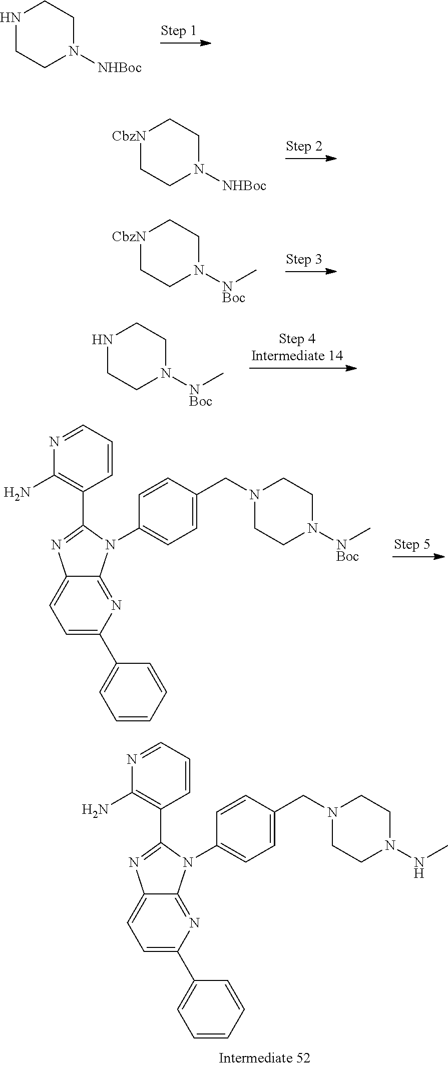

### Step 1: Benzyl 4-((tert-butoxycarbonyl)amino)piperazine-1-carboxylate

To a solution of tert-butyl N-piperazin-1-ylcarbamate (1 g, 4.98 mmol) in DMF (5 mL) were added TEA (1.39 mL, 9.96 mmol) and CbzCl (934 mg, 5.5 mmol) at 0° C. The mixture was stirred at 25° C. for 3 hr. The mixture was quenched with H₂O (35 mL) and extracted with EtOAc (30 mL×3). The combined organic layers were washed with brine (30 mL), dried over anhydrous Na₂SO₄, filtered and concentrated. After purified by silica gel flash chromatography (EtOAc in petroleum ether=0% to 60%), benzyl 4-((tert-butoxycarbonyl)amino)piperazine-1-carboxylate (1.2 g, 72% yield) was obtained as an off white solid. MS: m/z=358.1 [M+23]⁺.

### Step 2: Benzyl 4-((tert-butoxycarbonyl)(methyl)amino)piperazine-1-carboxylate

To a mixture of benzyl 4-((tert-butoxycarbonyl)amino)piperazine-1-carboxylate (1.2 g, 3.58 mmol) in THE (10 mL) was added NaH (429 mg, 60% purity) at 0° C. After stirring at 0° C. for 30 min, CH₃I (1.02 g, 7.16 mmol) was added. The resulting mixture was stirred at 25° C. for 5 hr under N₂. The reaction was quenched with water (10 mL) at 0° C. and extracted with CH₂Cl₂ (20 mL×3). The combined organic layers were washed with brine (30 mL), dried over anhydrous Na₂SO₄, filtered and concentrated. After purified by silica gel flash chromatography (EtOAc in petroleum ether=0% to 20%), benzyl 4-((tert-butoxycarbonyl)(methyl)amino)piperazine-1-carboxylate (1.1 g, yield: 88%) was obtained as a colorless oil. MS: m/z=372.1 [M+23]⁺.

### Step 3: tert-Butyl methyl(piperazin-1-yl)carbamate

To a mixture of benzyl 4-((tert-butoxycarbonyl)(methyl)amino)piperazine-1-carboxylate (1.1 g, 3.15 mmol) in MeOH (5 mL) was added Pd/C (1.1 g, 10% purity). The mixture was stirred at 25° C. for 12 hr under H₂ (50 psi). The mixture was filtered and concentrated to give tert-butyl methyl(piperazin-1-yl)carbamate (670 mg, yield: 99%) as an off-white solid, which was used into the next step without purification. MS: m/z=160.0 [M−C₄H₇]+. ¹H NMR (400 MHz, Methanol-d₄) δ 2.93-2.92 (m, 11H), 1.48 (s, 9H).

### Step 4: tert-Butyl (4-(4-(2-(2-aminopyridin-3-yl)-5-phenyl-3H-imidazo[4,5-b]pyridin-3-yl)benzyl)piperazin-1-yl)(methyl)carbamate

To a solution of Intermediate 14 (800 mg, 1.94 mmol) and tert-butyl methyl(piperazin-1-yl)carbamate (460 mg, 2.14 mmol) in DMF (8 mL) were added NaI (58.2 mg, 388 gmol) and K₂CO₃ (537 mg, 3.88 mmol). The mixture was stirred at 50° C. for 5 hr. The reaction was concentrated, diluted with water (10 mL), and extracted with EtOAc (20 mL×2). The combined organic layers were dried over anhydrous Na₂SO₄, filtered and concentrated. After purified by silica gel flash chromatography (CH₂Cl₂ in MeOH=0% to 2%), tert-butyl (4-(4-(2-(2-aminopyridin-3-yl)-5-phenyl-3H-imidazo[4,5-b]pyridin-3-yl)benzyl)piperazin-1-yl)(methyl)carbamate (0.35 g, yield: 30%) was obtained as a light yellow solid. MS: m/z=591.4 [M+1]⁺. ¹H NMR (400 MHz, Dimethylsulfoxide-d₆) 8.27 (d, J=8.0 Hz, 1H), 8.05-7.96 (m, 4H), 7.48-7.44 (m, 6H), 7.43-7.37 (m, 1H), 7.15 (dd, J=8.0, 2.0 Hz, 1H), 7.03 (s, 2H), 6.42-6.33 (m, 1H), 5.76-5.75 (m, 1H), 3.58 (s, 2H), 3.32-3.31 (m, 2H), 2.99-2.88 (brs, 3H), 2.85 (s, 3H), 2.47 (br s, 3H), 1.41 (s, 9H).

### Step 5: 4-(4-(2-(2-Aminopyridin-3-yl)-5-phenyl-3H-imidazo[4,5-b]pyridin-3-yl)benzyl)-N-methylpiperazin-1-amine

To a solution of tert-butyl (4-(4-(2-(2-aminopyridin-3-yl)-5-phenyl-3H-imidazo[4,5-b]pyridin-3-yl)benzyl)piperazin-1-yl)(methyl)carbamate (347 mg, 587 mol) in 1,4-dioxane (3 mL) was added HCl/1,4-dioxane (4 M, 147 L). The mixture was stirred at 25° C. for 5 hr under N₂. The mixture was concentrated to give 4-(4-(2-(2-aminopyridin-3-yl)-5-phenyl-3H-imidazo[4,5-b]pyridin-3-yl)benzyl)-N-methylpiperazin-1-amine (Intermediate 52, 288 mg, yield: 93.1%, HCl salt) as a yellow solid, which was used in the next step without further purification. MS: m/z=491.3 [M+H]⁺. ¹H NMR (400 MHz, Dimethylsulfoxide-d₆) 11.6 (br s, 1H), 11.2 (br s, 2H), 8.59-8.43 (m, 2H), 8.39-8.37 (m, 1H), 8.15-8.09 (m, 1H), 8.08-8.07 (m, 3H), 7.89-7.87 (m, 1H), 7.92-7.87 (m, 2H), 7.83-7.67 (m, 2H), 7.56-7.38 (m, 4H), 6.95-6.88 (m, 1H), 3.71-3.41 (m, 10H), 2.68 (br s, 3H).

### Intermediate 53: 2-(4-(2-(2-Aminopyridin-3-yl)-5-phenyl-3H-imidazo[4,5-b]pyridin-3-yl)benzyl)-2-azaspiro[3.5]nonan-7-amine

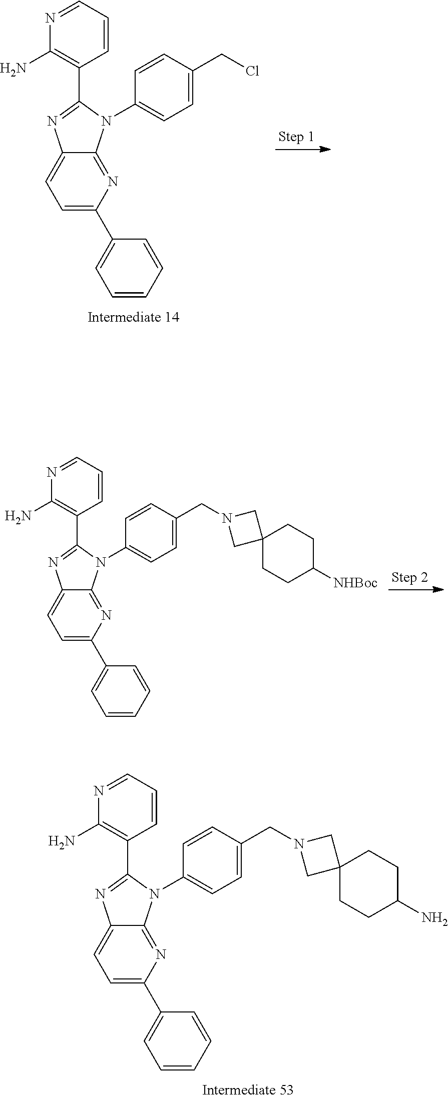 [SMILES: Nc1ncccc1-c1nc2ccc(-c3ccccc3)nc2n1-c1ccc(CCl)cc1 ; CC(C)(C)OC(=O)NC1CCC2(CC1)CN(Cc1ccc(-n3c(-c4cccnc4N)nc4ccc(-c5ccccc5)nc43)cc1)C2 ; Nc1ncccc1-c1nc2ccc(-c3ccccc3)nc2n1-c1ccc(CN2CC3(CCC(N)CC3)C2)cc1]

### Step 1: tert-Butyl (2-(4-(2-(2-aminopyridin-3-yl)-5-phenyl-3H-imidazo[4,5-b]pyridin-3-yl)benzyl)-2-azaspiro[3.5]nonan-7-yl)carbamate

To a solution of Intermediate 14 (1.0 g, 2.4 mmol) and tert-butyl (2-azaspiro[3.5]nonan-7-yl)carbamate (642 mg, 2.7 mmol) in DMF (15 mL) were added NaI (36 mg, 243 μmol) and K₂CO₃ (671 mg, 4.9 mmol). The mixture was stirred at 80° C. for 2 hr. The reaction mixture was quenched with H₂O (50 mL) at 25° C. and extracted with EtOAc (50 mL×2). The combined organic layers were washed with brine (50 mL), dried over Na₂SO₄, filtered, and concentrated under reduced pressure. The crude was purified by silica gel flash chromatography (Eluent of 0~10% MeOH in CH₂Cl₂) to give tert-butyl (2-(4-(2-(2-aminopyridin-3-yl)-5-phenyl-3H-imidazo[4,5-b]pyridin-3-yl)benzyl)-2-azaspiro[3.5]nonan-7-yl)carbamate (900 mg, yield: 60%) as a yellow solid. MS: m/z=616.4 [M+H]⁺. ¹H NMR (400 MHz, Chloroform-d) δ 8.12 (d, J=8.4 Hz, 1H), 8.06 (d, J=5.2 Hz, 1H), 8.02 (d, J=7.6 Hz, 2H), 7.80 (d, J=8.4 Hz, 1H), 7.48-7.36 (m, 7H), 7.09 (d, J=8.0 Hz, 1H), 6.59 (br s, 2H), 6.36 (dd, J=7.6, 4.8 Hz, 1H), 4.46-4.29 (m, 1H), 3.77 (s, 2H), 3.49-3.34 (m, 1H), 3.26-3.01 (m, 4H), 1.99-1.92 (m, 2H), 1.91-1.84 (m, 2H), 1.59-1.52 (m, 2H), 1.44 (s, 9H), 1.21-1.09 (m, 2H).

### Step 2: 2-(4-(2-(2-Aminopyridin-3-yl)-5-phenyl-3H-imidazo[4,5-b]pyridin-3-yl)benzyl)-2-azaspiro[3.5]nonan-7-amine

A solution of tert-butyl(2-(4-(2-(2-aminopyridin-3-yl)-5-phenyl-3H-imidazo[4,5-b]pyridin-3-yl)benzyl)-2-azaspiro[3.5]nonan-7-yl)carbamate (375 mg, 609 mol) in HCl in 1,4-dioxane (4M, 5 mL) was stirred at 25° C. for 1 hr. The mixture was concentrated under reduced pressure to give 2-(4-(2-(2-aminopyridin-3-yl)-5-phenyl-3H-imidazo[4,5-b]pyridin-3-yl)benzyl)-2-azaspiro[3.5]nonan-7-amine (Intermediate 53, 291 mg, HCl salt, yield: 87%) as a gray solid. MS: m/z=516.3 [M+H]⁺. ¹H NMR (400 MHz, Dimethylsulfoxide-d₆) δ 8.26 (d, J=8.4 Hz, 1H), 8.04-7.97 (m, 4H), 7.49-7.44 (m, 2H), 7.43-7.37 (m, 5H), 7.16 (dd, J=7.6, 1.6 Hz, 1H), 7.00 (br s, 2H), 6.38 (dd, J=7.6, 4.8 Hz, 1H), 3.65 (s, 2H), 3.20-3.12 (m, 1H), 2.95 (s, 2H), 2.90 (s, 2H), 1.86-1.80 (m, 2H), 1.65-1.57 (m, 2H), 1.42-1.34 (m, 2H), 1.11-0.95 (m, 2H).

### Intermediate 54: 3-(3-(4-((3,9-Diazaspiro[5.5]undecan-3-yl)methyl)phenyl)-5-phenyl-3H-imidazo[4,5-b]pyridin-2-yl)pyridin-2-amine

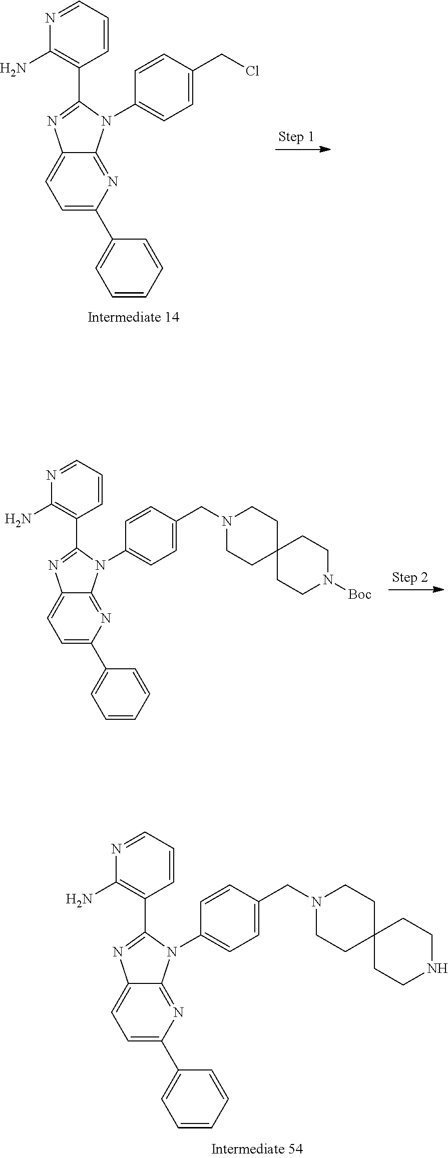 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)CC2)CC1 ; Nc1ncccc1-c1nc2ccc(-c3ccccc3)nc2n1-c1ccc(CN2CCC3(CCNCC3)CC2)cc1]

### Step 1: tert-Butyl 9-(4-(2-(2-aminopyridin-3-yl)-5-phenyl-3H-imidazo[4,5-b]pyridin-3-yl)benzyl)-3,9-diazaspiro[5.5]undecane-3-carboxylate

To a solution of Intermediate 14 (600 mg, 1.46 mmol), tert-butyl 3,9-diazaspiro[5.5]undecane-3-carboxylate (408 mg, 1.60 mmol) in DMF (5 mL) were added NaI (21.9 mg, 146 mol) and K₂CO₃ (403 mg, 2.91 mmol). The mixture was stirred at 80° C. for 16 hr. The reaction mixture was poured into H₂O (15 mL), extracted with EtOA (20 mL×3). The combined organic layers were dried over anhydrous Na₂SO₄, filtered, and concentrated under reduced pressure. The residue was purified by silica gel flash chromatography (Eluent of 0~65% EtOAc in petroleum ether) to give tert-butyl 9-(4-(2-(2-aminopyridin-3-yl)-5-phenyl-3H-imidazo[4,5-b]pyridin-3-yl)benzyl)-3,9-diazaspiro[5.5]undecane-3-carboxylate (450 mg, yield: 49%) as a yellow solid. MS: m/z=630.5 [M+H]⁺.

### Step 2: 3-(3-(4-((3,9-Diazaspiro[5.5]undecan-3-yl)methyl)phenyl)-5-phenyl-3H-imidazo[4,5-b]pyridin-2-yl)pyridin-2-amine

To a solution of 9-(4-(2-(2-aminopyridin-3-yl)-5-phenyl-3H-imidazo[4,5-b]pyridin-3-yl)benzyl)-3,9-diazaspiro[5.5]undecane-3-carboxylate (200 mg, 318 μmol) in 1,4-dioxane (2 mL) was added 4M HCl in 1,4-dioxane (2 mL). The mixture was stirred at 25° C. for 1 hr. The reaction mixture was filtered. The filter cake was dried to give 3-(3-(4-((3,9-diazaspiro[5.5]undecan-3-yl)methyl)phenyl)-5-phenyl-3H-imidazo[4,5-b]pyridin-2-yl)pyridin-2-amine (Intermediate 54, 121 mg, HCl salt, yield: 67%) as a yellow solid. MS: m/z=530.3 [M+H]⁺. ¹H NMR (400 MHz, Dimethylsulfoxide-d₆) δ 10.81-10.50 (m, 1H), 8.79-8.66 (m, 2H), 8.38 (dd, J=8.4 Hz, 1H), 8.13-8.11 (m, 1H), 8.08-8.05 (m, 4H), 7.86-7.77 (m, 3H), 7.67 (d, J=8.0 Hz, 2H), 7.51-7.46 (m, 2H), 7.45-7.41 (m, 1H), 6.86-6.78 (m, 1H), 4.41 (d, J=4.4 Hz, 2H), 3.21-3.15 (m, 2H), 3.09-3.01 (m, 6H), 1.91-1.85 (m, 2H), 1.82-1.71 (m, 4H), 1.54-1.53 (m, 2H).

### Intermediate 55: 3-(3-(4-((4-((Methyl-d₃)amino)piperidin-1-yl)methyl)phenyl)-5-phenyl-3H-imidazo[4,5-b]pyridin-2-yl)pyridin-2-amine

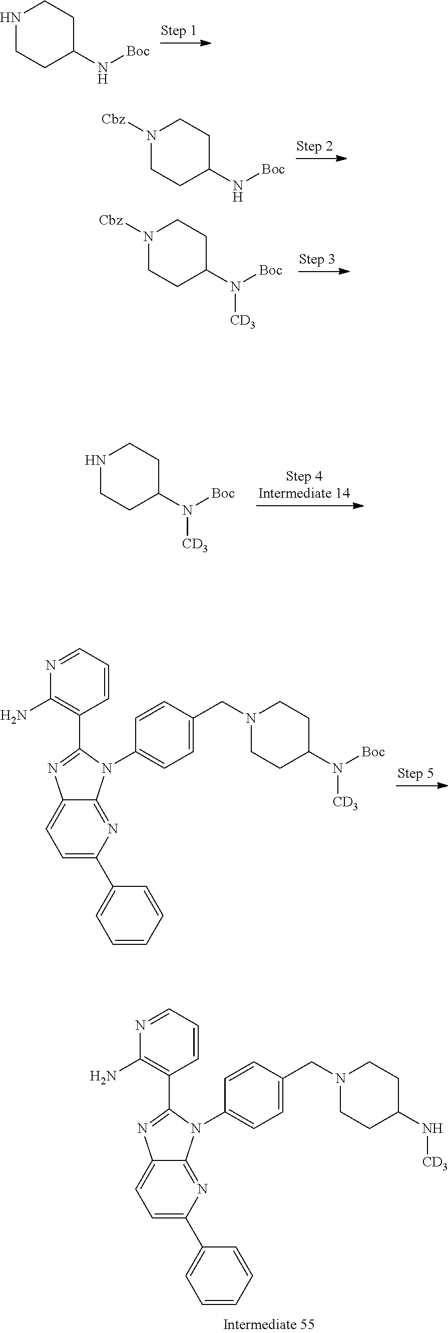

### Step 1: Benzyl 4-((tert-butoxycarbonyl)amino)piperidine-1-carboxylate

To a solution of tert-butyl N-(4-piperidyl)carbamate (12 g, 59.9 mmol) in CH₂Cl₂ (100 mL) were added TEA (18.2 g, 179 mmol), and then CbzCl (11.2 g, 65.9 mmol) was added into the mixture at 0° C. The mixture was stirred at 25° C. for 2 hr. The mixture was concentrated under reduced pressure. The crude product was purified by silica gel flash chromatography (Eluent of 10~50% EtOAc in petroleum ether) to give benzyl 4-((tert-butoxycarbonyl)amino)piperidine-1-carboxylate (16 g, yield: 71%) as an off-white solid. ¹H NMR (400 MHz, Dimethylsulfoxide-d₆) δ 7.40-7.28 (m, 5H), 5.06 (s, 2H), 3.90 (d, J=13.6 Hz, 2H), 3.50-3.34 (m, 2H), 2.89 (s, 2H), 1.71 (d, J=10.8 Hz, 2H), 1.37 (s, 9H), 1.30-1.18 (m, 2H).

### Step 2: Benzyl 4-((tert-butoxycarbonyl)(methyl-d₃)amino)piperidine-1-carboxylate

To a solution of benzyl 4-(tert-butoxycarbonylamino)piperidine-1-carboxylate (13 g, 38.9 mmol) in THF (200 mL) was added NaH (4.66 g, 117 mmol) at 0° C. After stirring at 0° C. for 30 min, CD₃I (16.5 g, 117 mmol) was added to the mixture. The mixture was stirred at 25° C. for 16 hr. The mixture was quenched with NH₄Cl (aq) (100 mL) at 0° C. The mixture was diluted with H₂O (100 mL) and extrated with CH₂Cl₂ (200 mL×2). The combined organic layers were washed with brine (200 mL×2), dried over Na₂SO₄, filtered and concentrated under reduced pressure. The residue was purified by silica gel flash chromatography (Eluent of 0~20% EtOAc in petroleum ether) to give benzyl 4-((tert-butoxycarbonyl)(methyl-d₃)amino)piperidine-1-carboxylate (8.6 g, yield: 56%) as a colorless oil. MS: m/z=252.3 [M+H−100]⁺. ¹H NMR (400 MHz, Dimethylsulfoxide-d₆) δ 7.42-7.28 (m, 5H), 5.07 (s, 2H), 4.14-3.89 (m, 3H), 2.82 (s, 2H), 1.58-1.48 (m, 4H), 1.39 (s, 9H).

### Step 3: tert-Butyl (methyl-d₃)(piperidin-4-yl)carbamate

To a solution of benzyl 4-[tert-butoxycarbonyl(trideuteriomethyl)amino]piperidine-1-carboxylate (8.6 g, 24.5 mmol) in MeOH (90 mL) was added Pd/C (900 mg, 24.5 mmol). The mixture was stirred at 25° C. for 16 hr under H₂ (15 psi). The mixture was filtered, and the filter cake was washed by MeOH (30 mL). The filtrate was concentrated under reduced pressure to give a tert-butyl (methyl-d₃)(piperidin-4-yl)carbamate (5 g, yield: 80%) as a colorless oil. MS: m/z=218.3 [M+H]⁺. 1H NMR (400 MHz, Dimethylsulfoxide-d₆) δ 3.93-3.62 (m, 1H), 3.26-3.16 (m, 1H), 2.95 (d, J=12.0 Hz, 2H), 2.47-2.38 (m, 2H), 1.54-1.42 (m, 4H), 1.39 (s, 9H).

### Step 4: tert-Butyl (1-(4-(2-(2-aminopyridin-3-yl)-5-phenyl-3H-imidazo[4,5-b]pyridin-3-yl)benzyl)piperidin-4-yl)(methyl-d₃)carbamate

To a solution of Intermediate 14 (1 g, 2.43 mmol) in DMF (10 mL) were added tert-butyl (methyl-d₃)(piperidin-4-yl)carbamate (527 mg, 2.43 mmol), NaI (182 mg, 1.21 mmol) and K₂CO₃ (1.0 g, 7.28 mmol). The mixture was stirred at 80° C. for 18 hr. The reaction mixture was added H₂O (20 mL) and extracted with CH₂Cl₂ (20 mL×3). The combined organic layers were washed with brine (20 mL×2), dried over Na₂SO₄, filtered and concentrated under reduced pressure. The residue was purified by silica gel flash chromatography (Eluent of 0~6% MeOH in CH₂Cl₂) to give tert-butyl (1-(4-(2-(2-Aminopyridin-3-yl)-5-phenyl-3H-imidazo[4,5-b]pyridin-3-yl)benzyl)piperidin-4-yl)(methyl-d₃)carbamate (750 mg, yield: 49%) as a yellow solid. MS: m/z=593.3 [M+H]⁺.

### Step 5: 3-(3-(4-((4-((Methyl-d₃)amino)piperidin-1-yl)methyl)phenyl)-5-phenyl-3H-imidazo[4,5-b]pyridin-2-yl)pyridin-2-amine

To a solution of tert-butyl (1-(4-(2-(2-aminopyridin-3-yl)-5-phenyl-3H-imidazo[4,5-b]pyridin-3-yl)benzyl)piperidin-4-yl)(methyl-d3)carbamate (430 mg, 725 μmol) in CH₂Cl₂ (5 mL) was added TFA (165 mg, 1.45 mmol). The mixture was stirred at 25° C. for 1 hr. The mixture was diluted with H₂O (10 mL). The pH of the mixture was adjusted to about 8 by NaHCO₃ (aq.). The mixture was extracted with CH₂Cl₂ (10 mL×2). The combined organic layers were washed with brine (10 mL×2), dried over Na₂SO₄, filtered, and concentrated under reduced pressure. After purified by prep-TLC (CH₂Cl₂:MeOH=5:1), 3-(3-(4-((4-((methyl-d₃)amino)piperidin-1-yl)methyl)phenyl)-5-phenyl-3H-imidazo[4,5-b]pyridin-2-yl)pyridin-2-amine (Intermediate 55, 350 mg crude, yield: 50%) was obtained as a light-yellow solid. MS: m/z=493.3 [M+H]⁺. ¹H NMR (400 MHz, Methanol-d₄) δ 8.19 (d, J=8.4 Hz, 1H), 8.03 (d, J=7.2 Hz, 2H), 7.98 (dd, J=4.8, 1.6 Hz, 1H), 7.93 (d, J=8.4 Hz, 1H), 7.53 (d, J=8.4 Hz, 2H), 7.47-7.35 (m, 5H), 7.32 (dd, J=7.6, 1.6 Hz, 1H), 6.47 (dd, J=7.6, 4.8 Hz, 1H), 3.65 (s, 2H), 3.08-3.03 (m, 2H), 3.02-2.95 (m, 1H), 2.21-2.15 (m, 2H), 2.08-2.05 (m, 2H), 1.67-1.61 (m, 2H).

### Intermediate 56: 3-(3-(4-((2,8-Diazaspiro[4.5]decan-2-yl)methyl)phenyl)-5-phenyl-3H-imidazo[4,5-b]pyridin-2-yl)pyridin-2-amine

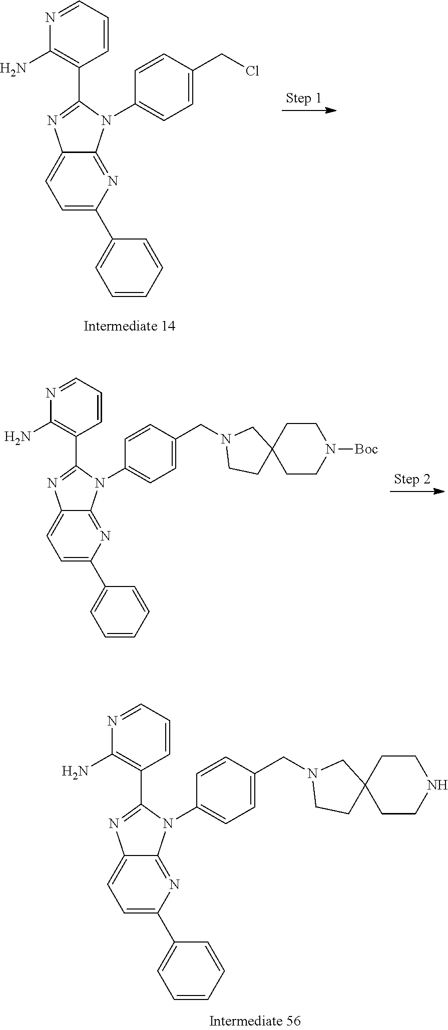 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)C2)CC1 ; Nc1ncccc1-c1nc2ccc(-c3ccccc3)nc2n1-c1ccc(CN2CCC3(CCNCC3)C2)cc1]

### Step 1: tert-Butyl 2-(4-(2-(2-aminopyridin-3-yl)-5-phenyl-3H-imidazo[4,5-b]pyridin-3-yl)benzyl)-2,8-diazaspiro[4.5]decane-8-carboxylate

To a solution of Intermediate 14 (1.5 g, 3.6 mmol) and tert-butyl 2,8-diazaspiro[4.5]decane-8-carboxylate (1.0 g, 4.4 mmol) in DMF (10 mL) were added NaI (273 mg, 1.8 mmol) and K₂CO₃ (1.0 g, 7.0 mmol). The mixture was stirred at 80° C. for 1 hr. After cooling to 20° C., the reaction mixture was poured into H₂O (100 mL) and extracted with EtOAc (50 mL×3). The combined organic layers were dried over anhydrous Na₂SO₄, filtered, and concentrated under reduced pressure. The residue was purified by silica gel flash chromatography (Eluent of 0-85% EtOAc in petroleum ether) to give tert-butyl 2-(4-(2-(2-aminopyridin-3-yl)-5-phenyl-3H-imidazo[4,5-b]pyridin-3-yl)benzyl)-2,8-diazaspiro[4.5]decane-8-carboxylate (720 mg, yield: 34%) as a yellow solid. MS: m/z=616.4 [M+H]⁺.

### Step 2: 3-(3-(4-((2,8-Diazaspiro[4.5]decan-2-yl)methyl)phenyl)-5-phenyl-3H-imidazo[4,5-b]pyridin-2-yl)pyridin-2-amine

A solution of tert-butyl 2-(4-(2-(2-aminopyridin-3-yl)-5-phenyl-3H-imidazo[4,5-b]pyridin-3-yl)benzyl)-2,8-diazaspiro[4.5]decane-8-carboxylate (720 mg, 1.2 mmol) in HCl in 1,4-dioxane (4 M, 5 mL) was stirred at 25° C. for 0.5 hr. The mixture was concentrated and washed with CH₂Cl₂ (3 mL), then concentrated to dryness under reduced pressure. 3-(3-(4-((2,8-Diazaspiro[4.5]decan-2-yl)methyl)phenyl)-5-phenyl-3H-imidazo[4,5-b]pyridin-2-yl)pyridin-2-amine (Intermediate 56, 600 mg, HCl salt, yield: 91%) was obtained as a yellow solid. MS: m/z=516.3 [M+H]⁺. ¹H NMR (400 MHz, Methanol-d₄) δ 8.33 (d, J=8.8 Hz, 1H), 8.10-8.01 (m, 4H), 7.96-7.87 (m, 3H), 7.72 (d, J=8.0 Hz, 2H), 7.49-7.40 (m, 3H), 6.96-6.87 (m, 1H), 4.67-4.54 (m, 2H), 3.75-3.65 (m, 2H), 3.51 (d, J=10.0 Hz, 1H), 3.35 (s, 2H), 3.32-3.20 (m, 3H), 2.32-2.23 (m, 1H), 2.15-2.05 (m, 2H), 2.04-1.96 (m, 3H).

### Example 1: 3-(4-(4-(2-(2-Aminopyridin-3-yl)-3H-imidazo[4,5-b]pyridin-3-yl)benzyl)piperazin-1-yl)-4-methoxycyclobut-3-ene-1,2-dione

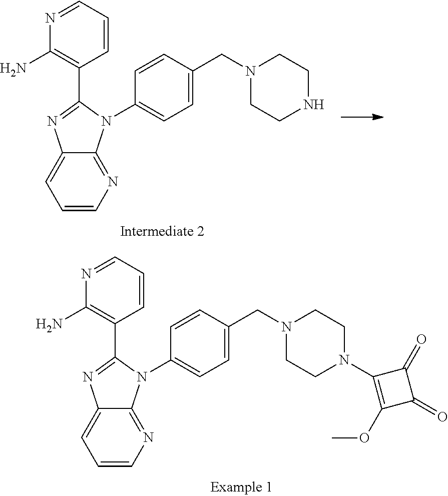 [SMILES: Nc1ncccc1-c1nc2cccnc2n1-c1ccc(CN2CCNCC2)cc1 ; COc1c(N2CCN(Cc3ccc(-n4c(-c5cccnc5N)nc5cccnc54)cc3)CC2)c(=O)c1=O]

To a solution of Intermediate 2 (200 mg, 519 μmol) in MeOH (4 mL) were added TEA (2.5 mmol) and 3,4-dimethoxycyclobut-3-ene-1,2-dione (88.5 mg, 623 μmol). The reaction mixture stirred at 25° C. for 16 hr. The mixture was then filtered and the collected solid residue was washed with MeOH (10 mL×2) and dried in vacuo to give 3-(4-(4-(2-(2-aminopyridin-3-yl)-3H-imidazo[4,5-b]pyridin-3-yl)benzyl)piperazin-1-yl)-4-methoxycyclobut-3-ene-1,2-dione (Example 1, 150 mg, yield: 54%) as a light-yellow solid. MS: m/z=496.2 [M+H]⁺. ¹H NMR (400 MHz, Dimethylsulfoxide-d₆) δ 8.33 (dd, J=4.8, 1.2 Hz, 1H), 8.20 (dd, J=8.0, 1.6 Hz, 1H), 7.98 (dd, J=4.8, 1.6 Hz, 1H), 7.46 (d, J=8.0 Hz, 2H), 7.43-7.36 (m, 3H), 7.16 (dd, J=7.6, 1.6 Hz, 1H), 6.99 (br s, 2H), 6.38 (dd, J=7.6, 4.8 Hz, 1H), 4.30 (s, 3H), 3.86-3.76 (m, 2H), 3.62 (s, 2H), 3.60-3.45 (m, 2H), 2.57-2.51 (m, 4H).

### Example 2: 3-((4-(2-(2-Aminopyridin-3-yl)-3H-imidazo[4,5-b]pyridin-3-yl)benzyl)amino)-4-methoxycyclobut-3-ene-1,2-dione

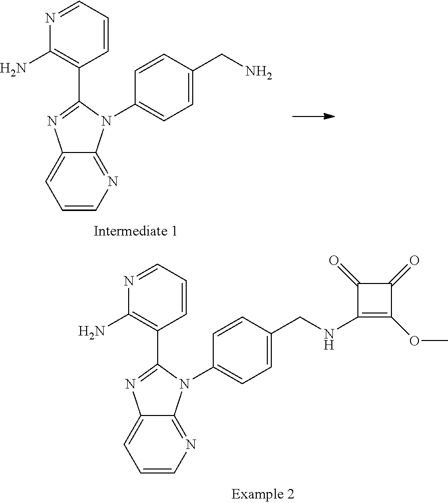 [SMILES: NCc1ccc(-n2c(-c3cccnc3N)nc3cccnc32)cc1 ; COc1c(NCc2ccc(-n3c(-c4cccnc4N)nc4cccnc43)cc2)c(=O)c1=O]

Following the general procedure of Example 1, the reaction of Intermediate 1 (200 mg, 632 μmol) with 3,4-dimethoxycyclobut-3-ene-1,2-dione (108 mg, 759 μmol) was carried out. After prep-TLC (CH₂Cl₂:MeOH=10:1), 3-((4-(2-(2-aminopyridin-3-yl)-3H-imidazo[4,5-b]pyridin-3-yl)benzyl)amino)-4-methoxycyclobut-3-ene-1,2-dione (Example 2, 50 mg, yield: 11%, 1:1 mixture of tautomers) was obtained as an off-white solid. MS: m/z=427.0 [M+H]⁺. ¹H NMR (400 MHz, Dimethylsulfoxide-d₆) δ 9.35-9.28 (m, 0.5H), 9.18-9.05 (m, 0.5H), 8.31 (dd, J=4.8, 1.2 Hz, 1H), 8.20 (dd, J=8.0, 1.2 Hz, 1H), 7.99 (dd, J=4.8, 1.6 Hz, 1H), 7.47-7.41 (m, 4H), 7.39 (dd, J=8.0, 4.8 Hz, 1H), 7.22 (dd, J=7.6, 1.6 Hz, 1H), 6.92 (br s, 2H), 6.40 (dd, J=7.6, 4.8 Hz, 1H), 4.77 (d, J=5.6 Hz, 1H), 4.55 (d, J=6.0 Hz, 1H), 4.30 (s, 3H).

### Example 3: 3-((1-(4-(2-(2-Aminopyridin-3-yl)-3H-imidazo[4,5-b]pyridin-3-yl)benzyl)piperidin-4-yl)amino)-4-methoxycyclobut-3-ene-1,2-dione

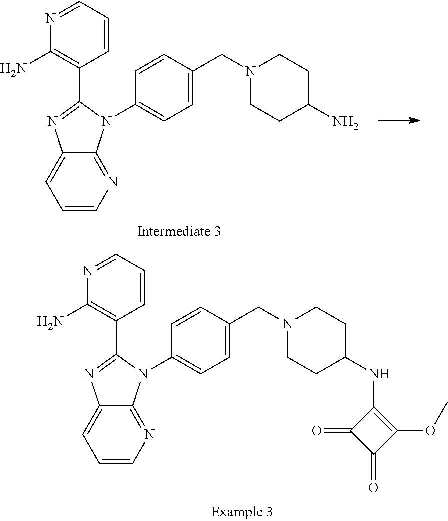 [SMILES: Nc1ncccc1-c1nc2cccnc2n1-c1ccc(CN2CCC(N)CC2)cc1 ; COc1c(NC2CCN(Cc3ccc(-n4c(-c5cccnc5N)nc5cccnc54)cc3)CC2)c(=O)c1=O]

Following the general procedure of Example 1, the reaction of Intermediate 3 (200 mg, 500 μmol) with 3,4-dimethoxycyclobut-3-ene-1,2-dione (85.4 mg, 600 mol) was carried out and 3-((1-(4-(2-(2-aminopyridin-3-yl)-3H-imidazo[4,5-b]pyridin-3-yl)benzyl)piperidin-4-yl)amino)-4-methoxycyclobut-3-ene-1,2-dione (Example 3, 120 mg, yield: 44%, 6:4 mixture of tautomers) was obtained as an orange solid. MS: m/z=510.2 [M+H]⁺. ¹H NMR (400 MHz, Dimethylsulfoxide-d₆) δ 8.86 (d, J=8.4 Hz, 0.6H), 8.65 (d, J=8.4 Hz, 0.4H), 8.33 (dd, J=4.8, 1.6 Hz, 1H), 8.20 (dd, J=8.0, 1.2 Hz, 1H), 7.99 (dd, J=4.8, 1.6 Hz, 1H), 7.48-7.30 (m, 5H), 7.16 (dd, J=7.7, 1.6 Hz, 1H), 7.01 (br s, 2H), 6.37 (t, J=6.0 Hz, 1H), 4.31-4.27 (m, 3H), 3.87-3.78 (m, 0.4H), 3.54 (s, 2H), 3.42-3.38 (m, 0.6H), 2.86-2.77 (m, 2H), 2.07-1.94 (m, 2H), 1.86-1.78 (m, 2H), 1.62-1.50 (m, 2H).

### Example 4: 3-((4-(2-(2-Aminopyridin-3-yl)-5-phenyl-3H-imidazo[4,5-b]pyridin-3-yl)benzyl)amino)-4-methoxycyclobut-3-ene-1,2-dione

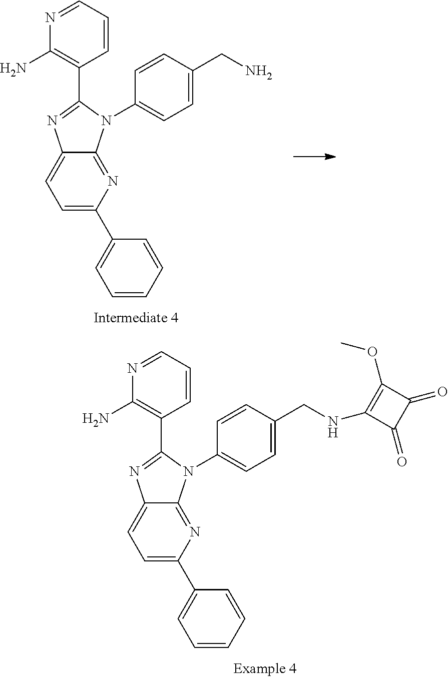 [SMILES: NCc1ccc(-n2c(-c3cccnc3N)nc3ccc(-c4ccccc4)nc32)cc1 ; COc1c(NCc2ccc(-n3c(-c4cccnc4N)nc4ccc(-c5ccccc5)nc43)cc2)c(=O)c1=O]

Following the general procedure of Example 1, the reaction of Intermediate 4 (200 mg, 510 μmol) with 3,4-dimethoxycyclobut-3-ene-1,2-dione (86.9 mg, 612 mol) was carried out. After prep-TLC (CH₂Cl₂:MeOH=10:1), 3-((4-(2-(2-aminopyridin-3-yl)-5-phenyl-3H-imidazo[4,5-b]pyridin-3-yl)benzyl)amino)-4-methoxycyclobut-3-ene-1,2-dione (Example 4, 13.0 mg, yield: 4.6%, 1:1 mixture of tautomers) was obtained as a brown solid. MS: m/z=503.1 [M+H]⁺. ¹H NMR (400 MHz, Dimethylsulfoxide-d₆) δ 9.37-9.27 (m, 0.5H), 9.17-9.06 (m, 0.5H), 8.27 (d, J=8.4 Hz, 1H), 8.05-7.97 (m, 4H), 7.51-7.38 (m, 7H), 7.21 (d, J=7.6 Hz, 1H), 6.92 (br s, 2H), 6.42 (dd, J=7.6, 4.8 Hz, 1H), 4.85-4.76 (m, 1H), 4.62-4.54 (m, 1H), 4.30 (s, 3H).

### Example 5: 3-((4-(2-(2-Aminopyridin-3-yl)-5-phenyl-3H-imidazo[4,5-b]pyridin-3-yl)benzyl)amino)-4-hydroxycyclobut-3-ene-1,2-dione

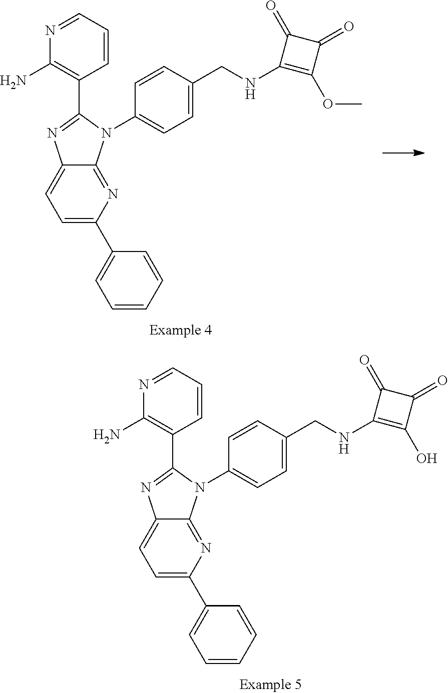 [SMILES: COc1c(NCc2ccc(-n3c(-c4cccnc4N)nc4ccc(-c5ccccc5)nc43)cc2)c(=O)c1=O ; Nc1ncccc1-c1nc2ccc(-c3ccccc3)nc2n1-c1ccc(CNc2c(O)c(=O)c2=O)cc1]

To a solution of Example 4 (200 mg, 398 μmol) in EtOH (4 mL) at 0° C. was added NaOH (79.5 mg, 1.99 mmol) in H₂O (2 mL) dropwise. The reaction mixture was stirred at 25° C. for 16 hr. The mixture was concentrated under reduced pressure. The residue was purified by prep-HPLC (column: Waters xbridge 150×25 mm 10 μm; mobile phase: [water (NH₄HCO₃)-ACN]; B %: 26%-56%, 8 min) to give 3-((4-(2-(2-aminopyridin-3-yl)-5-phenyl-3H-imidazo[4,5-b]pyridin-3-yl)benzyl)amino)-4-hydroxycyclobut-3-ene-1,2-dione (Example 5, 30 mg, yield: 15%, mixture of tautomers) as a white solid. MS: m/z=489.1 [M+H]⁺. ¹H NMR (400 MHz, Dimethylsulfoxide-d₆) δ 8.26 (d, J=8.4 Hz, 1H), 8.05-7.96 (m, 4H), 7.93-7.82 (m, 1H), 7.51-7.43 (m, 6H), 7.42-7.36 (m, 1H), 7.22 (dd, J=7.6, 2.0 Hz, 1H), 7.08 (s, 0.5H), 6.98 (br s, 2H), 6.94 (s, 0.5H), 6.43 (dd, J=7.6, 4.8 Hz, 1H), 4.71 (d, J=6.4 Hz, 2H).

### Example 6: N-(3-(2-(2-Aminopyridin-3-yl)-3-(4-(((2-methoxy-3,4-dioxocyclobut-1-en-1-yl)amino)methyl)phenyl)-3H-imidazo[4,5-b]pyridin-5-yl)phenyl)acetamide

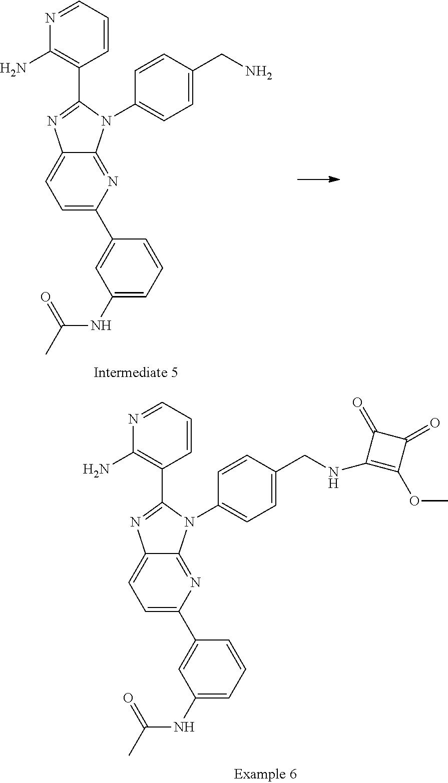 [SMILES: CC(=O)Nc1cccc(-c2ccc3nc(-c4cccnc4N)n(-c4ccc(CN)cc4)c3n2)c1 ; COc1c(NCc2ccc(-n3c(-c4cccnc4N)nc4ccc(-c5cccc(NC(C)=O)c5)nc43)cc2)c(=O)c1=O]

Following the general procedure of Example 1, the reaction of Intermediate 5 (170 mg, 378 μmol) with 3,4-dimethoxycyclobut-3-ene-1,2-dione (64.5 mg, 454 μmol) was carried out. N-(3-(2-(2-aminopyridin-3-yl)-3-(4-(((2-methoxy-3,4-dioxocyclobut-1-en-1-yl)amino)methyl)phenyl)-3H-imidazo[4,5-b]pyridin-5-yl)phenyl)acetamide (Example 6, 150 mg, yield: 67%, 1:1 mixture of tautomers) was obtained as a brown solid. MS: m/z=560.1 [M+H]⁺. ¹H NMR (400 MHz, Dimethylsulfoxide-d₆) δ 10.06 (s, 1H), 9.38-9.30 (m, 0.5H), 9.19-9.06 (m, 0.5H), 8.28 (d, J=8.4 Hz, 1H), 8.12 (d, J=12.0 Hz, 1H), 8.00 (dd, J=4.8, 2.0 Hz, 1H), 7.85 (d, J=8.4 Hz, 1H), 7.72-7.63 (m, 2H), 7.54-7.45 (m, 4H), 7.38 (dd, J=7.6, 7.6 Hz, 1H), 7.21 (d, J=7.6 Hz, 1H), 6.88 (br s, 2H), 6.41 (dd, J=7.6, 4.8 Hz, 1H), 4.80 (d, J=5.6 Hz, 1H), 4.70 (d, J=6.0 Hz, 1H), 4.30 (s, 3H), 2.05 (s, 3H).

### Example 7: N-(3-(2-(2-Aminopyridin-3-yl)-3-(4-((4-(2-methoxy-3,4-dioxocyclobut-1-en-1-yl)piperazin-1-yl)methyl)phenyl)-3H-imidazo[4,5-b]pyridin-5-yl)phenyl)acetamide

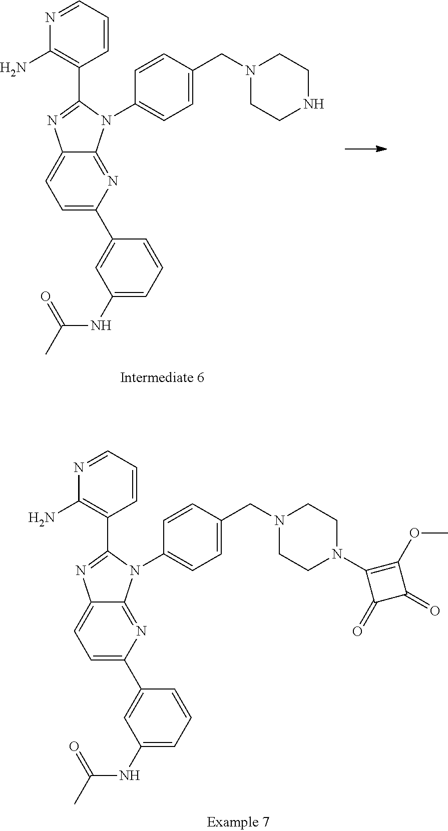 [SMILES: CC(=O)Nc1cccc(-c2ccc3nc(-c4cccnc4N)n(-c4ccc(CN5CCNCC5)cc4)c3n2)c1 ; COc1c(N2CCN(Cc3ccc(-n4c(-c5cccnc5N)nc5ccc(-c6cccc(NC(C)=O)c6)nc54)cc3)CC2)c(=O)c1=O]

Following the general procedure of Example 1, the reaction of Intermediate 6 (160 mg, 309 μmol) with 3,4-dimethoxycyclobut-3-ene-1,2-dione (52.6 mg, 370 μmol) was carried out. N-(3-(2-(2-aminopyridin-3-yl)-3-(4-((4-(2-methoxy-3,4-dioxocyclobut-1-en-1-yl)piperazin-1-yl)methyl)phenyl)-3H-imidazo[4,5-b]pyridin-5-yl)phenyl)acetamide (Example 7, 110 mg, yield: 53%) was obtained as a brown solid. MS: m/z=629.1 [M+H]⁺. ¹H NMR (400 MHz, Dimethylsulfoxide-d₆) δ 10.04 (s, 1H), 8.27 (d, J=8.4 Hz, 1H), 8.20 (s, 1H), 7.99 (dd, J=4.8, 1.6 Hz, 1H), 7.87 (d, J=8.4 Hz, 1H), 7.70-7.60 (m, 2H), 7.51-7.44 (m, 4H), 7.38 (dd, J=8.0, 8.0 Hz, 1H), 7.16 (dd, J=7.6, 1.6 Hz, 1H), 6.98 (br s, 2H), 6.39 (dd, J=7.6, 4.8 Hz, 1H), 4.30 (s, 3H), 3.86-3.77 (m, 2H), 3.63 (s, 2H), 3.59-3.52 (m, 2H), 2.58-2.54 (m, 4H), 2.05 (s, 3H).

### Example 8: N-(3-(2-(2-Aminopyridin-3-yl)-3-(4-((4-((2-methoxy-3,4-dioxocyclobut-1-en-1-yl)amino)piperidin-1-yl)methyl)phenyl)-3H-imidazo[4,5-b]pyridin-5-yl)phenyl)acetamide

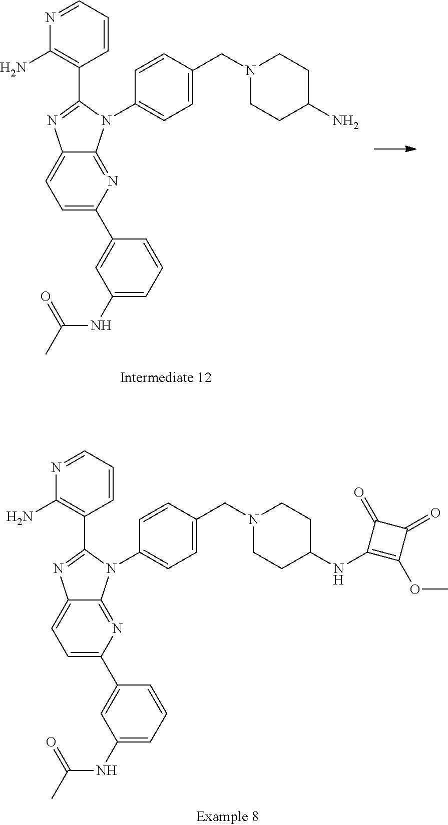 [SMILES: CC(=O)Nc1cccc(-c2ccc3nc(-c4cccnc4N)n(-c4ccc(CN5CCC(N)CC5)cc4)c3n2)c1 ; COc1c(NC2CCN(Cc3ccc(-n4c(-c5cccnc5N)nc5ccc(-c6cccc(NC(C)=O)c6)nc54)cc3)CC2)c(=O)c1=O]

Following the general procedure of Example 1, the reaction of Intermediate 12 (200 mg, 375 μmol) with 3,4-dimethoxycyclobut-3-ene-1,2-dione (64.0 mg, 450 μmol) was carried out. After prep-HPLC (column: Waters Xbridge C18 150×25 mm×10 μm, mobile phase: [water (NH₃H₂O)-ACN]; B %: 10%-30%, 6 min) and prep-TLC (CH₂Cl₂:MeOH=10:1), N-(3-(2-(2-aminopyridin-3-yl)-3-(4-((4-((2-methoxy-3,4-dioxocyclobut-1-en-1-yl)amino)piperidin-1-yl)methyl)phenyl)-3H-imidazo[4,5-b]pyridin-5-yl)phenyl)acetamide (Example 8, 10 mg, yield: 4.0%, 1:1 mixture of tautomers) was obtained as a yellow solid. MS: m/z=643.1 [M+H]⁺. ¹H NMR (400 MHz, Chloroform-d) δ 8.13-8.03 (m, 3H), 7.77 (d, J=8.4 Hz, 1H), 7.72 (d, J=7.6 Hz, 1H), 7.68-7.57 (m, 2H), 7.50-7.33 (m, 5H), 7.05 (d, J=7.2 Hz, 1H), 6.61 (br s, 2H), 6.52 (br s, 1H), 6.34 (dd, J=7.6, 4.8 Hz, 1H), 4.43-4.33 (m, 3H), 3.57 (s, 3H), 2.93-2.84 (m, 2H), 2.24-2.17 (m, 2H), 2.14 (s, 3H), 1.99-1.94 (m, 2H), 1.70-1.58 (m, 2H).

### Example 9: 3-(4-(4-(2-(2-Aminopyridin-3-yl)-6-phenyl-3H-imidazo[4,5-b]pyridin-3-yl)benzyl)piperazin-1-yl)-4-methoxycyclobut-3-ene-1,2-dione

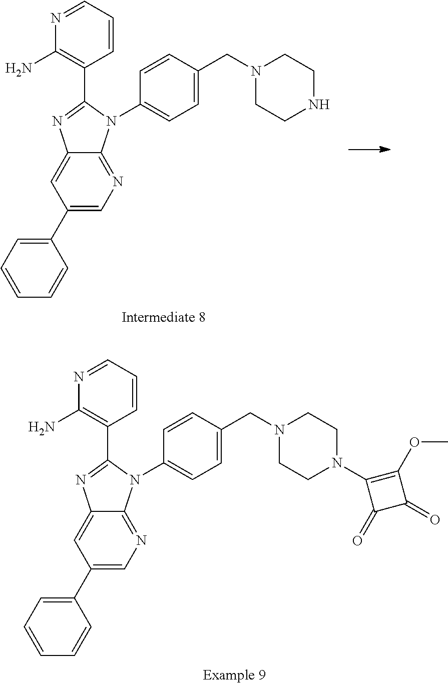 [SMILES: Nc1ncccc1-c1nc2cc(-c3ccccc3)cnc2n1-c1ccc(CN2CCNCC2)cc1 ; COc1c(N2CCN(Cc3ccc(-n4c(-c5cccnc5N)nc5cc(-c6ccccc6)cnc54)cc3)CC2)c(=O)c1=O]

Following the general procedure of Example 1, the reaction of Intermediate 8 (200 mg, 433 μmol) with 3,4-dimethoxycyclobut-3-ene-1,2-dione (73.9 mg, 520 mol) was carried out. 3-(4-(4-(2-(2-aminopyridin-3-yl)-6-phenyl-3H-imidazo[4,5-b]pyridin-3-yl)benzyl)piperazin-1-yl)-4-methoxycyclobut-3-ene-1,2-dione (Example 9, 165 mg, yield: 61%) was obtained as a yellow solid. MS: m/z=572.1 [M+H]⁺. ¹H NMR (400 MHz, Dimethylsulfoxide-d₆) δ 8.63 (d, J=1.8 Hz, 1H), 8.47 (d, J=1.8 Hz, 1H), 8.02-7.98 (m, 1H), 7.84-7.77 (m, 2H), 7.55-7.46 (m, 4H), 7.45-7.39 (m, 3H), 7.21-7.17 (m, 1H), 7.04 (br s, 2H), 6.40 (dd, J=7.6, 4.8 Hz, 1H), 4.30 (s, 3H), 3.88-3.74 (m, 2H), 3.63 (s, 2H), 3.58-3.50 (m, 2H), 2.57-2.53 (m, 4H).

### Example 10: 3-((1-(4-(2-(2-Aminopyridin-3-yl)-6-phenyl-3H-imidazo[4,5-b]pyridin-3-yl)benzyl)piperidin-4-yl)amino)-4-methoxycyclobut-3-ene-1,2-dione

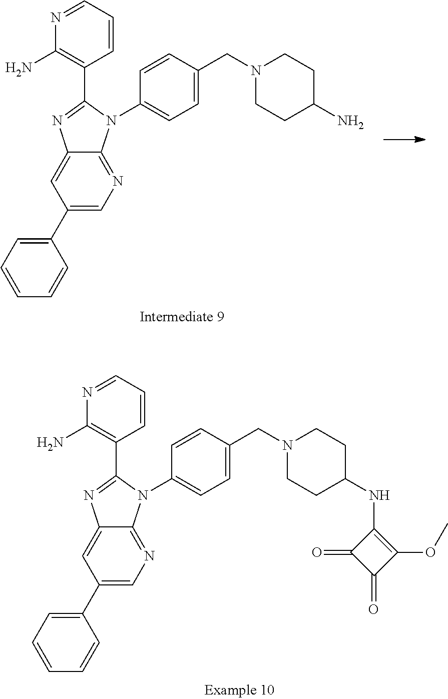 [SMILES: Nc1ncccc1-c1nc2cc(-c3ccccc3)cnc2n1-c1ccc(CN2CCC(N)CC2)cc1 ; COc1c(NC2CCN(Cc3ccc(-n4c(-c5cccnc5N)nc5cc(-c6ccccc6)cnc54)cc3)CC2)c(=O)c1=O]

Following the general procedure of Example 1, the reaction of Intermediate 9 (200 mg, 420 μmol) with 3,4-dimethoxycyclobut-3-ene-1,2-dione (71.7 mg, 504 μmol) was carried out. 3-((1-(4-(2-(2-aminopyridin-3-yl)-6-phenyl-3H-imidazo[4,5-b]pyridin-3-yl)benzyl)piperidin-4-yl)amino)-4-methoxycyclobut-3-ene-1,2-dione (Example 10, 50 mg, yield: 19.5%, 1:1 mixture of tautomers) was obtained as a yellow solid. MS: m/z=586.3 [M+H]⁺. ¹H NMR (400 MHz, Chloroform-d) δ 8.62 (d, J=2.0 Hz, 1H), 8.27 (d, J=2.0 Hz, 1H), 8.06 (dd, J=4.8, 1.6 Hz, 1H), 7.64 (d, J=7.2 Hz, 2H), 7.53-7.48 (m, 4H), 7.44-7.35 (m, 3H), 7.09 (dd, J=7.6, 1.2 Hz, 1H), 6.66 (br s, 2H), 6.50-6.38 (m, 1H), 6.35 (dd, J=7.6, 4.8 Hz, 1H), 4.42 (s, 3H), 3.61 (s, 2H), 3.60-3.51 (m, 1H), 2.95-2.85 (m, 2H), 2.26-2.17 (m, 2H), 2.03-1.93 (m, 2H), 1.73-1.61 (m, 2H).

### Example 11: 3-((1-(4-(2-(2-aminopyridin-3-yl)-5-phenyl-3H-imidazo[4,5-b]pyridin-3-yl)benzyl)piperidin-4-yl)amino)-4-isopropoxycyclobut-3-ene-1,2-dione

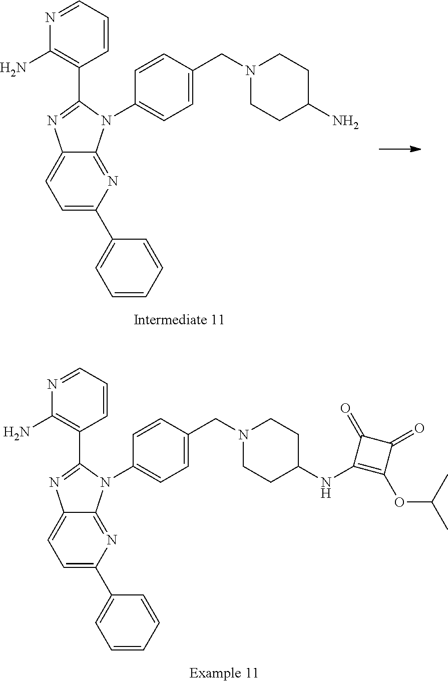 [SMILES: Nc1ncccc1-c1nc2ccc(-c3ccccc3)nc2n1-c1ccc(CN2CCC(N)CC2)cc1 ; CC(C)Oc1c(NC2CCN(Cc3ccc(-n4c(-c5cccnc5N)nc5ccc(-c6ccccc6)nc54)cc3)CC2)c(=O)c1=O]

To a solution of Intermediate 11 (100 mg, 210 μmol) and TEA (1.05 mmol) in MeOH (4 mL) was added 3,4-diisopropoxycyclobut-3-ene-1,2-dione (50 mg, 252 μmol). The reaction mixture stirred at 25° C. for 16 hr. The mixture was concentrated under reduced pressure and purified by prep-TLC (MeOH:CH₂Cl₂=1:10) to give 3-((1-(4-(2-(2-aminopyridin-3-yl)-5-phenyl-3H-imidazo[4,5-b]pyridin-3-yl)benzyl)piperidin-4-yl)amino)-4-isopropoxycyclobut-3-ene-1,2-dione (Example 11, 18.9 mg, yield: 14%, 1:1 mixture of tautomers) as a light-yellow powder. MS: m/z=614.2 [M+H]⁺. ¹H NMR (400 MHz, Dimethylsulfoxide-d₆) δ 8.86 (d, J=8.0 Hz, 0.5H), 8.65 (d, J=8.0 Hz, 0.5H), 8.27 (d, J=8.4 Hz, 1H), 8.05-7.97 (m, 4H), 7.49-7.43 (m, 6H), 7.42-7.37 (m, 1H), 7.15 (d, J=8.0 Hz, 1H), 7.03 (br s, 2H), 6.41-6.35 (m, 1H), 5.29-5.19 (m, 1H), 3.89-3.79 (m, 0.5H), 3.57 (s, 2H), 3.48-3.41 (m, 0.5H), 2.90-2.80 (m, 2H), 2.10-1.98 (m, 2H), 1.86-1.75 (m, 2H), 1.63-1.54 (m, 2H), 1.38 (t, J=5.6 Hz, 6H).

### Example 12: 3-((1-(4-(2-(2-Aminopyridin-3-yl)-5-phenyl-3H-imidazo[4,5-b]pyridin-3-yl)benzyl)piperidin-4-yl)amino)-4-ethoxycyclobut-3-ene-1,2-dione

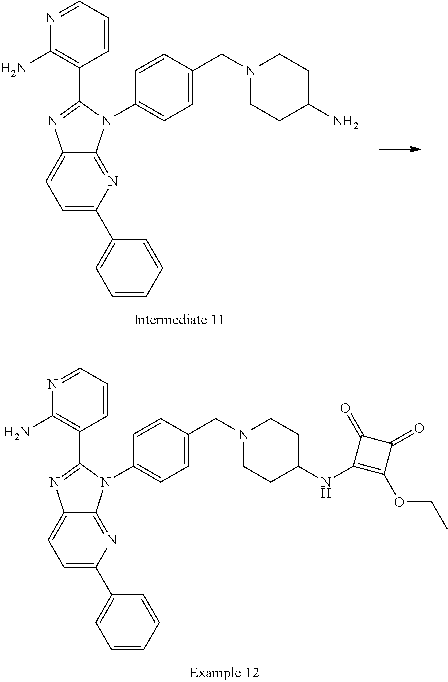 [SMILES: Nc1ncccc1-c1nc2ccc(-c3ccccc3)nc2n1-c1ccc(CN2CCC(N)CC2)cc1 ; CCOc1c(NC2CCN(Cc3ccc(-n4c(-c5cccnc5N)nc5ccc(-c6ccccc6)nc54)cc3)CC2)c(=O)c1=O]

Following the general procedure for Example 11, the reaction of Intermediate 11 (100 mg, 210 μmol) with 3,4-diethoxycyclobut-3-ene-1,2-dione (42.9 mg, 252 μmol) was carried. After prep-TLC (MeOH:CH₂Cl₂=1:10), 3-((1-(4-(2-(2-aminopyridin-3-yl)-5-phenyl-3H-imidazo[4,5-b]pyridin-3-yl)benzyl)piperidin-4-yl)amino)-4-ethoxycyclobut-3-ene-1,2-dione (Example 12, 11.3 mg, yield: 8.8%, 1:1 mixture of tautomers) was obtained as a light-yellow powder. MS: m/z=600.3 [M+H]⁺. ¹H NMR (400 MHz, Dimethylsulfoxide-d₆) δ 8.87 (d, J=8.0 Hz, 0.5H), 8.66 (d, J=8.4 Hz, 0.5H), 8.26 (d, J=8.4 Hz, 1H), 8.05-7.96 (m, 4H), 7.49-7.43 (m, 6H), 7.42-7.37 (m, 1H), 7.17-7.12 (m, 1H), 7.03 (br s, 2H), 6.41-6.34 (m, 1H), 4.66 (q, J=7.2 Hz, 2H), 3.89-3.76 (m, 0.5H), 3.57 (s, 2H), 3.43-3.41 (m, 0.5H), 2.90-2.78 (m, 2H), 2.11-1.95 (m, 2H), 1.89-1.75 (m, 2H), 1.63-1.53 (m, 2H), 1.36 (t, J=7.2 Hz, 3H).

### Example 13: 3-((1-(4-(2-(2-Aminopyridin-3-yl)-5-phenyl-3H-imidazo[4,5-b]pyridin-3-yl)benzyl)piperidin-4-yl)amino)-4-(methoxy-d3)cyclobut-3-ene-1,2-dione

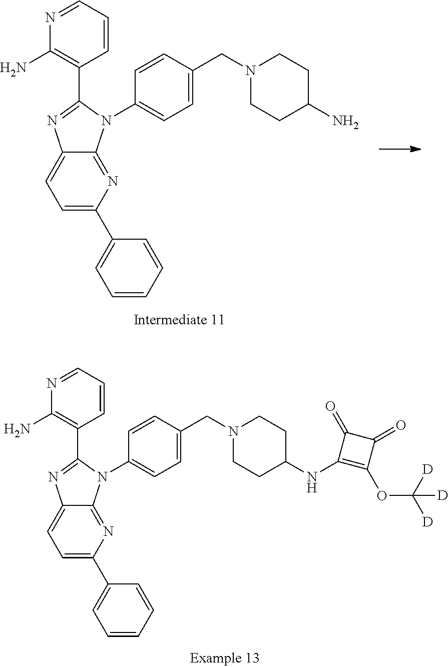 [SMILES: Nc1ncccc1-c1nc2ccc(-c3ccccc3)nc2n1-c1ccc(CN2CCC(N)CC2)cc1 ; [2H]C([2H])([2H])Oc1c(NC2CCN(Cc3ccc(-n4c(-c5cccnc5N)nc5ccc(-c6ccccc6)nc54)cc3)CC2)c(=O)c1=O]

Following the general procedure for Example 11, the reaction of Intermediate 11 (100 mg, 210 μmol) with 3,4-bis(trideuteriomethoxy)cyclobut-3-ene-1,2-dione (46.7 mg, 315 μmol) was carried out. After prep-TLC (MeOH: CH₂Cl₂=1:10), 3-((1-(4-(2-(2-aminopyridin-3-yl)-5-phenyl-3H-imidazo[4,5-b]pyridin-3-yl)benzyl)piperidin-4-yl)amino)-4-(methoxy-d3)cyclobut-3-ene-1,2-dione (Example 13, 17.3 mg, yield: 13%, 1:1 mixture of tautomers) was obtained as a light-yellow solid. MS: m/z=589.3 [M+H]⁺. ¹H NMR (400 MHz, Dimethylsulfoxide-d₆) δ 8.86 (d, J=8.4 Hz, 0.5H), 8.65 (d, J=7.6 Hz, 0.5H), 8.26 (d, J=8.0 Hz, 1H), 8.04-7.96 (m, 4H), 7.48-7.42 (m, 6H), 7.41-7.37 (m, 1H), 7.15 (d, J=7.6 Hz, 1H), 7.02 (br s, 2H), 6.38-6.37 (m, 1H), 3.89-3.76 (m, 0.5H), 3.57 (s, 2H), 3.17 (d, J=4.8 Hz, 0.5H), 2.88-2.77 (m, 2H), 2.06-2.03 (m, 2H), 1.88-1.71 (m, 2H), 1.63-1.54 (m, 2H).

### Example 14: 3-((1-(4-(2-(2-Aminopyridin-3-yl)-5-phenyl-3H-imidazo[4,5-b]pyridin-3-yl)benzyl)piperidin-4-yl)amino)-4-(cyclopentyloxy)cyclobut-3-ene-1,2-dione

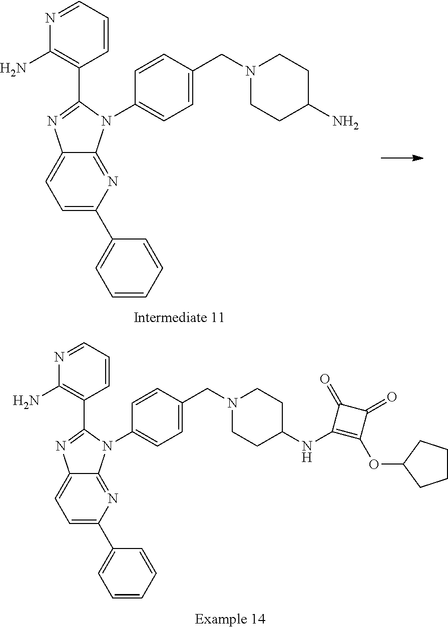 [SMILES: Nc1ncccc1-c1nc2ccc(-c3ccccc3)nc2n1-c1ccc(CN2CCC(N)CC2)cc1 ; Nc1ncccc1-c1nc2ccc(-c3ccccc3)nc2n1-c1ccc(CN2CCC(Nc3c(OC4CCCC4)c(=O)c3=O)CC2)cc1]

Following the general procedure for Example 11, the reaction of Intermediate 11 (100 mg, 210 μmol) with 3,4-bis(cyclopentoxy)cyclobut-3-ene-1,2-dione (63.1 mg, 252 μmol) was carried out. After silica gel flash chromatography (Eluent of 0~7% EtOAc in petroleum ether), 3-((1-(4-(2-(2-aminopyridin-3-yl)-5-phenyl-3H-imidazo[4,5-b]pyridin-3-yl)benzyl)piperidin-4-yl)amino)-4-(cyclopentyloxy)cyclobut-3-ene-1,2-dione (Example 14, 40.7 mg, yield: 30%, 1:1 mixture of tautomers) was obtained as a light-yellow powder. MS: m/z=640.4 [M+H]⁺. ¹H NMR (400 MHz, Dimethylsulfoxide-d₆) δ 8.87 (d, J=8.0 Hz, 0.5H), 8.60 (d, J=8.0 Hz, 0.5H), 8.27 (d, J=8.4 Hz, 1H), 8.09-7.93 (m, 4H), 7.53-7.42 (m, 6H), 7.42-7.36 (m, 1H), 7.15 (d, J=7.6 Hz, 1H), 7.03 (br s, 2H), 6.44-6.31 (m, 1H), 5.57-5.43 (m, 1H), 3.91-3.77 (m, 0.5H), 3.57 (s, 2H), 3.43-3.39 (m, 0.5H), 2.93-2.80 (m, 2H), 2.63-2.52 (m, 2H), 2.07-1.98 (m, 2H), 1.87-1.79 (m, 4H), 1.73-1.55 (m, 6H).

### Example 15: 2-((1-(4-(2-(2-Aminopyridin-3-yl)-5-phenyl-3H-imidazo[4,5-b]pyridin-3-yl)benzyl)piperidin-4-yl)amino)-3-(cyclopentyloxy)-4-thioxocyclobut-2-en-1-one

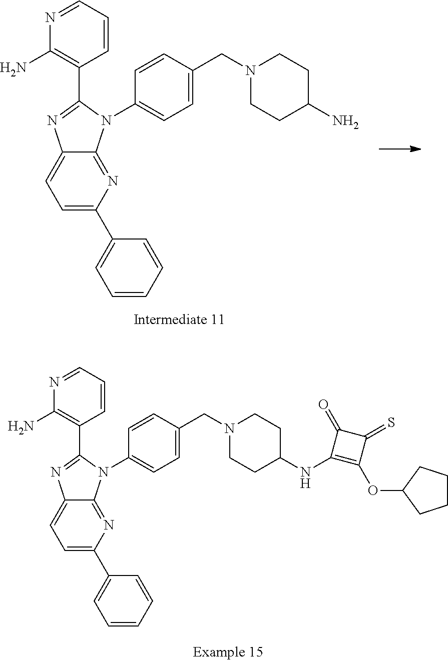 [SMILES: Nc1ncccc1-c1nc2ccc(-c3ccccc3)nc2n1-c1ccc(CN2CCC(N)CC2)cc1 ; Nc1ncccc1-c1nc2ccc(-c3ccccc3)nc2n1-c1ccc(CN2CCC(Nc3c(OC4CCCC4)c(=S)c3=O)CC2)cc1]

Following the general procedure for Example 11, the reaction of Intermediate 11 (100 mg, 210 μmol) with 2,3-bis(cyclopentyloxy)-4-thioxocyclobut-2-enone (138 mg, 630 μmol) was carried out. After prep-TLC (MeOH: CH₂Cl₂=1:10), 2-((1-(4-(2-(2-aminopyridin-3-yl)-5-phenyl-3H-imidazo[4,5-b]pyridin-3-yl)benzyl)piperidin-4-yl)amino)-3-(cyclopentyloxy)-4-thioxocyclobut-2-en-1-one (Example 15, 22.2 mg, yield: 16%, 1:1 mixture of tautomers) was obtained as a yellow solid. MS: m/z=656.4 [M+H]⁺. ¹H NMR (400 MHz, Dimethylsulfoxide-d₆) δ 9.83 (d, J=8.0 Hz, 0.5H), 9.48 (d, J=8.0 Hz, 0.5H), 8.27 (d, J=8.4 Hz, 1H), 8.05-7.97 (m, 4H), 7.49-7.44 (m, 6H), 7.41-7.37 (m, 1H), 7.18-7.14 (m, 1H), 7.03 (br s, 2H), 6.40-6.35 (m, 1.5H), 6.30-6.26 (m, 0.5H), 3.90-3.77 (m, 0.5H), 3.58 (s, 2H), 3.51-3.47 (m, 0.5H), 2.89-2.84 (m, 2H), 2.07-1.95 (m, 4H), 1.90-1.84 (m, 4H), 1.73-1.62 (m, 6H).

### Example 16: 3-((1-(4-(2-(2-Aminopyridin-3-yl)-5-phenyl-3H-imidazo[4,5-b]pyridin-3-yl)benzyl)piperidin-4-yl)amino)-4-(cyclopentyloxy)cyclobut-3-ene-1,2-dithione

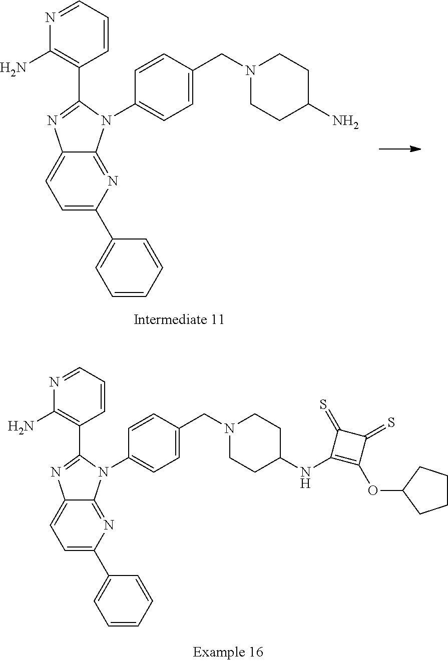 [SMILES: Nc1ncccc1-c1nc2ccc(-c3ccccc3)nc2n1-c1ccc(CN2CCC(N)CC2)cc1 ; Nc1ncccc1-c1nc2ccc(-c3ccccc3)nc2n1-c1ccc(CN2CCC(Nc3c(OC4CCCC4)c(=S)c3=S)CC2)cc1]

Following the general procedure for Example 11, the reaction of Intermediate 11 (100 mg, 210 μmol) with 3,4-bis(cyclopentyloxy)cyclobut-3-ene-1,2-dithione (178 mg, 630 μmol) was carried out. After silica gel flash chromatography (Eluent of 0~6% EtOAc in petroleum ether), 3-((1-(4-(2-(2-aminopyridin-3-yl)-5-phenyl-3H-imidazo[4,5-b]pyridin-3-yl)benzyl)piperidin-4-yl)amino)-4-(cyclopentyloxy)cyclobut-3-ene-1,2-dithione (Example 16, 11.5 mg, yield: 8.1%, 6:4 mixture of tautomers) was obtained as a yellow solid. MS: m/z=672.3 [M+H]⁺. ¹H NMR (400 MHz, Dimethylsulfoxide-d₆) δ 9.85 (d, J=8.0 Hz, 0.6H), 9.75 (d, J=7.6 Hz, 0.4H), 8.27 (d, J=8.4 Hz, 1H), 8.05-7.97 (m, 4H), 7.49-7.44 (m, 5H), 7.42-7.37 (m, 1H), 7.19-7.12 (m, 1H), 7.03 (br s, 2H), 6.48-6.29 (m, 2H), 4.88-4.60 (m, 0.4H), 3.58 (s, 2H), 3.46-3.38 (m, 0.6H), 2.96-2.83 (m, 2H), 2.04-1.94 (m, 6H), 1.90-1.84 (m, 2H), 1.77-1.63 (m, 6H).

### Example 17: 3-((1-(4-(2-(2-Aminopyridin-3-yl)-5-phenyl-3H-imidazo[4,5-b]pyridin-3-yl)benzyl)piperidin-4-yl)amino)-4-methoxycyclobut-3-ene-1,2-dione

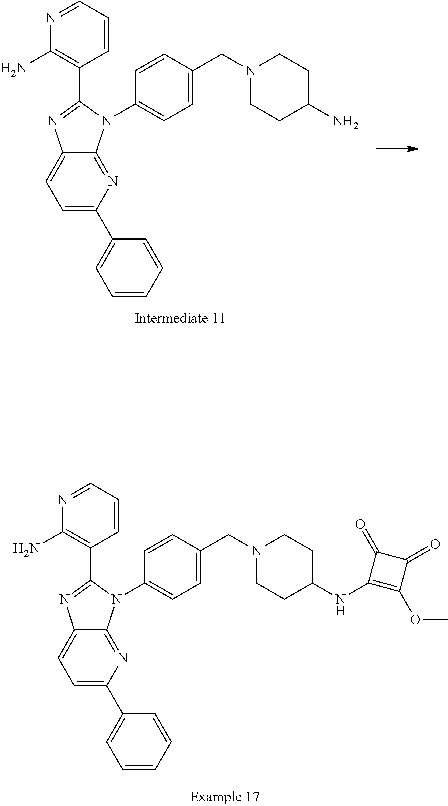 [SMILES: Nc1ncccc1-c1nc2ccc(-c3ccccc3)nc2n1-c1ccc(CN2CCC(N)CC2)cc1 ; COc1c(NC2CCN(Cc3ccc(-n4c(-c5cccnc5N)nc5ccc(-c6ccccc6)nc54)cc3)CC2)c(=O)c1=O]

Following the general procedure for Example 11, the reaction of Intermediate 11 (250 mg, 526 μmol) with 3,4-dimethoxycyclobut-3-ene-1,2-dione (89.6 mg, 631 μmol) was carried out. The mixture was filtered and the collected solid was washed with MeOH (10 mL×2) to give 3-((1-(4-(2-(2-Aminopyridin-3-yl)-5-phenyl-3H-imidazo[4,5-b]pyridin-3-yl)benzyl)piperidin-4-yl)amino)-4-methoxycyclobut-3-ene-1,2-dione (Intermediate 17, 84.4 mg, 27% yield, 1:1 mixture of tautomers)) as a brown solid. MS: m/z=586.2 [M+H]⁺. ¹H NMR (400 MHz, Dimethylsulfoxide-d₆) δ 8.86 (d, J=8.0 Hz, 0.5H), 8.65 (d, J=8.0 Hz, 1H), 8.27 (d, J=8.4 Hz, 1H), 8.04-7.97 (m, 4H), 7.48-7.31 (m, 7H), 7.15 (dd, J=7.6, 1.2 Hz, 1H). 7.04 (s, 2H), 6.39-6.36 (m, 1H), 4.29 (s, 3H), 3.84-3.78 (m, 0.5H), 3.57 (s, 2H), 3.45-3.40 (m, 0.5H), 2.84 (br d, J=10.8 Hz, 2H), 2.02-2.00 (m, 2H), 1.84-1.81 (m, 2H), 1.62-1.56 (m, 2H).

### Example 18: 3-((1-(4-(2-(2-Aminopyridin-3-yl)-5-phenyl-3H-imidazo[4,5-b]pyridin-3-yl)benzyl)piperidin-4-yl)amino)-4-hydroxycyclobut-3-ene-1,2-dione

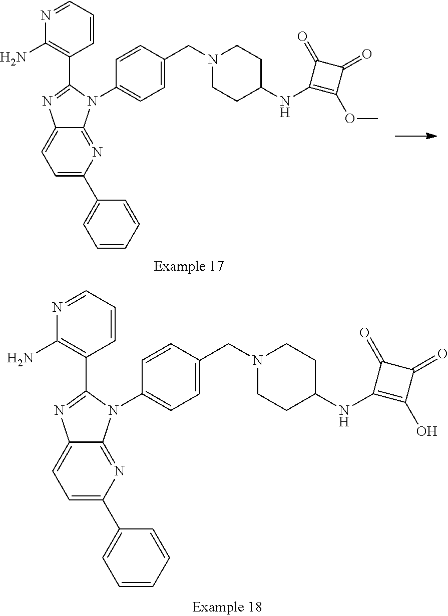 [SMILES: COc1c(NC2CCN(Cc3ccc(-n4c(-c5cccnc5N)nc5ccc(-c6ccccc6)nc54)cc3)CC2)c(=O)c1=O ; Nc1ncccc1-c1nc2ccc(-c3ccccc3)nc2n1-c1ccc(CN2CCC(Nc3c(O)c(=O)c3=O)CC2)cc1]

To a solution of Example 17 (100 mg, 170 mol) in EtOH (1 mL) was added NaOH (20.5 mg, 512 μmol) in 0.5 mL H₂O at 0° C. The mixture was stirred at 25° C. for 16 hr. The reaction was concentrated under reduced pressure. The residue was purified by prep-HPLC (column: Waters xbridge 150×25 mm 10 m; mobile phase: [water (NH₄HCO₃)-ACN]; B %: 7%-37%, 8 min) to give 3-((1-(4-(2-(2-aminopyridin-3-yl)-5-phenyl-3H-imidazo[4,5-b]pyridin-3-yl)benzyl)piperidin-4-yl)amino)-4-hydroxycyclobut-3-ene-1,2-dione (Example 18, 40 mg, yield: 39%) as a light-yellow solid. MS: m/z=572.2 [M+H]⁺. ¹H NMR (400 MHz, Methanol-d₄) δ 8.21 (d, J=8.4 Hz, 1H), 8.04-7.99 (m, 3H), 7.95 (d, J=8.4 Hz, 1H), 7.68 (d, J=8.4 Hz, 2H), 7.62 (d, J=8.4 Hz, 2H), 7.44-7.35 (m, 4H), 6.52 (dd, J=7.6, 4.8 Hz, 1H), 4.29 (s, 2H), 4.09-4.02 (m, 1H), 3.51-3.41 (m, 2H), 3.10-2.94 (m, 2H), 2.22-2.15 (m, 2H), 1.99-1.86 (m, 2H).

### Example 19: 3-((1-(4-(2-(2-Aminopyridin-3-yl)-5-phenyl-3H-imidazo[4,5-b]pyridin-3-yl)benzyl)piperidin-4-yl)amino)-4-(dimethylamino)cyclobut-3-ene-1,2-dione

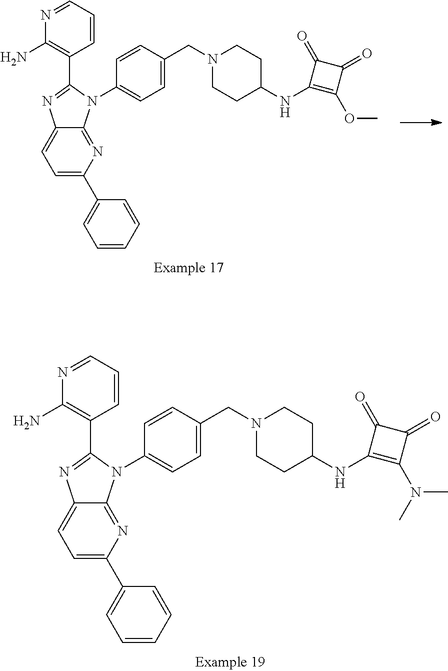 [SMILES: COc1c(NC2CCN(Cc3ccc(-n4c(-c5cccnc5N)nc5ccc(-c6ccccc6)nc54)cc3)CC2)c(=O)c1=O ; CN(C)c1c(NC2CCN(Cc3ccc(-n4c(-c5cccnc5N)nc5ccc(-c6ccccc6)nc54)cc3)CC2)c(=O)c1=O]

To a solution of Example 17 (50 mg, 85.4 μmol) in DMSO (0.15 mL) was added N-methylmethanamine (2 M in THF, 650 μL). The mixture was stirred at 25° C. for 1 hr. The reaction mixture was concentrated under reduced pressure. The residue was purified by prep-HPLC (column: Waters xbridge 150×25 mm 10 μm; mobile phase: [water (NH₄HCO₃)-ACN]; B %: 29%-59%, 8 min) to give 3-((1-(4-(2-(2-aminopyridin-3-yl)-5-phenyl-3H-imidazo[4,5-b]pyridin-3-yl)benzyl)piperidin-4-yl)amino)-4-(dimethylamino)cyclobut-3-ene-1,2-dione (Example 19, 20 mg, yield: 39%) as an off-white solid. MS: m/z=599.4 [M+H]⁺. ¹H NMR (400 MHz, Dimethylsulfoxide-d₆ δ 8.27 (d, J=8.4 Hz, 1H), 8.07-7.95 (m, 4H), 7.55-7.41 (m, 8H), 7.14 (dd, J=8.0, 1.6 Hz, 1H), 7.04 (br s, 2H), 6.38 (dd, J=8.0, 5.2 Hz, 1H), 4.15-4.02 (m, 1H), 3.59 (s, 2H), 3.16 (s, 6H), 2.91-2.84 (m, 2H), 2.07-1.99 (m, 2H), 1.87-1.79 (m, 2H), 1.71-1.59 (m, 2H).

### Example 20: 3-((1-(4-(2-(2-Aminopyridin-3-yl)-5-phenyl-3H-imidazo[4,5-b]pyridin-3-yl)benzyl)piperidin-4-yl)amino)-4-(methylamino)cyclobut-3-ene-1,2-dione

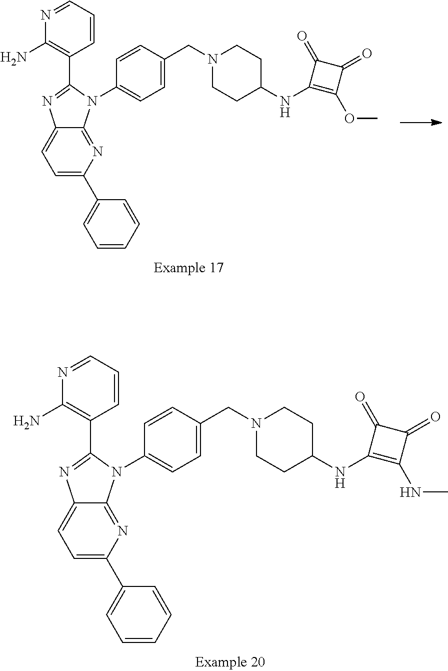 [SMILES: COc1c(NC2CCN(Cc3ccc(-n4c(-c5cccnc5N)nc5ccc(-c6ccccc6)nc54)cc3)CC2)c(=O)c1=O ; CNc1c(NC2CCN(Cc3ccc(-n4c(-c5cccnc5N)nc5ccc(-c6ccccc6)nc54)cc3)CC2)c(=O)c1=O]

To a solution of Example 17 (50 mg, 85.4 μmol) in MeOH (0.5 mL) and CHCl₃ (0.5 mL) was added MeNH₂ (1.0 mL, 30% purity in ethanol). The mixture was stirred at 25° C. for 2 hr. The reaction mixture was concentrated under reduced pressure. The residue was purified by prep-HPLC (column: Welch Xtimate C18 150×25 mm×5 μm; mobile phase: [water (HCl)-ACN]; B %: 2%-32%, 11 min) to give 3-((1-(4-(2-(2-aminopyridin-3-yl)-5-phenyl-3H-imidazo[4,5-b]pyridin-3-yl)benzyl)piperidin-4-yl)amino)-4-(methylamino)cyclobut-3-ene-1,2-dione (Example 20, 3HCl salt, 20 mg, yield: 40%, 1:1 mixture of tautomers) as a yellow solid. MS: m/z=584.9 [M+H]⁺. ¹H NMR (400 MHz, Dimethylsulfoxide-d₆ δ 10.89 (br s, 1H), 9.19-8.36 (m, 3H), 8.35-8.20 (m, 1H), 8.17-8.04 (m, 4H), 7.97-7.76 (m, 4H), 7.71-7.66 (m, 2H), 7.53-7.39 (m, 3H), 6.90 (dd, J=7.2, 6.4 Hz, 1H), 4.44 (d, J=4.8 Hz, 0.5H), 4.38 (d, J=4.2 Hz, 1.5H), 4.29-4.20 (m, 0.5H), 4.13-3.97 (m, 1.5H), 3.41-3.38 (m, 1H), 3.19-3.03 (m, 5H), 2.23-2.09 (m, 2H), 2.05-1.87 (m, 2H).

### Example 21: 3-(4-(4-(2-(2-Aminopyridin-3-yl)-5-phenyl-3H-imidazo[4,5-b]pyridin-3-yl)benzyl)piperazin-1-yl)-4-methoxycyclobut-3-ene-1,2-dione

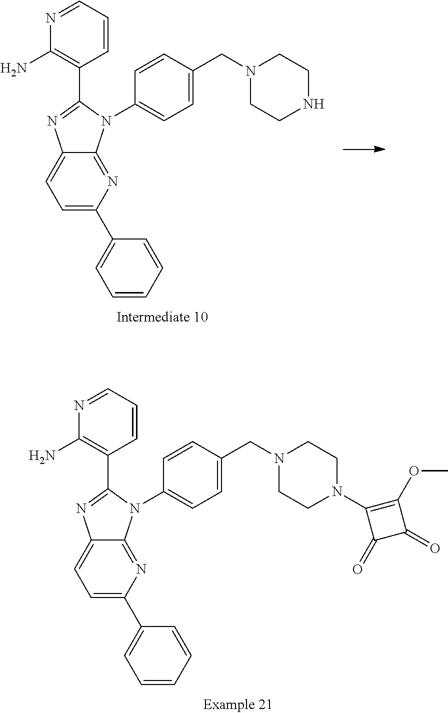 [SMILES: Nc1ncccc1-c1nc2ccc(-c3ccccc3)nc2n1-c1ccc(CN2CCNCC2)cc1 ; COc1c(N2CCN(Cc3ccc(-n4c(-c5cccnc5N)nc5ccc(-c6ccccc6)nc54)cc3)CC2)c(=O)c1=O]

To a solution of Intermediate 10 (200 mg, 433 μmol) in MeOH (4 mL) were added TEA (2.15 mmol) and dimethoxycyclobut-3-ene-1,2-dione (73.9 mg, 520 μmol). The reaction mixture was stirred at 25° C. for 16 hr. The mixture was filtered. The collected solid residue was washed with MeOH (10 mL×2) and dried in vacuo to give 3-(4-(4-(2-(2-aminopyridin-3-yl)-5-phenyl-3H-imidazo[4,5-b]pyridin-3-yl)benzyl)piperazin-1-yl)-4-methoxycyclobut-3-ene-1,2-dione (Example 21, 90 mg, yield: 34%) as a brown solid. MS: m/z=572.2 [M+H]⁺. ¹H NMR (400 MHz, Dimethylsulfoxide-d₆) δ 8.27 (d, J=8.4 Hz, 1H), 8.05-7.97 (m, 4H), 7.52-7.45 (m, 6H), 7.42-7.38 (m, 1H), 7.16 (dd, J=8.0, 2.0 Hz, 1H), 7.02 (br s, 2H), 6.39 (dd, J=7.6, 4.8 Hz, 1H), 4.30 (s, 3H), 3.85-3.77 (m, 2H), 3.64 (s, 2H), 3.58-3.53 (m, 2H), 2.59-2.53 (m, 4H).

### Example 22: 3-(4-(4-(2-(2-Aminopyridin-3-yl)-5-phenyl-3H-imidazo[4,5-b]pyridin-3-yl)benzyl)piperazin-1-yl)-4-hydroxycyclobut-3-ene-1,2-dione

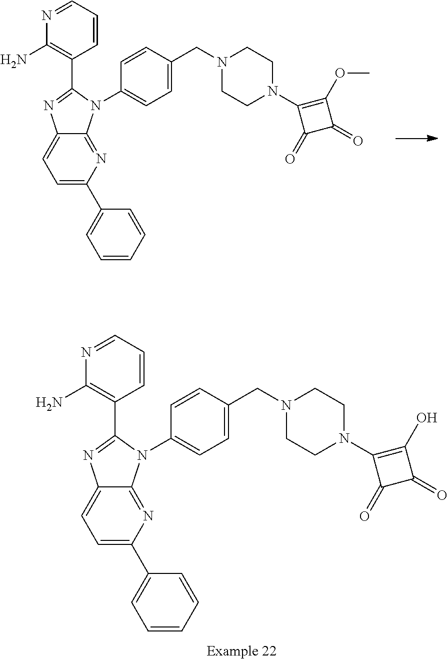 [SMILES: COc1c(N2CCN(Cc3ccc(-n4c(-c5cccnc5N)nc5ccc(-c6ccccc6)nc54)cc3)CC2)c(=O)c1=O ; Nc1ncccc1-c1nc2ccc(-c3ccccc3)nc2n1-c1ccc(CN2CCN(c3c(O)c(=O)c3=O)CC2)cc1]

To a solution of Example 21 (200 mg, 350 mol) in EtOH (4 mL) at 0° C. was added NaOH (42 mg, 1.05 mmol) in H₂O (0.8 mL) dropwise. The mixture was stirred at 25° C. for 16 hr. The reaction mixture was concentrated under reduced pressure. The residue was purified by prep-HPLC (column: Waters xbridge 150×25 mm 10 μm; mobile phase: [water (NH₄HCO₃)-ACN]; B %: 17%-47%, 8 min) to give 3-(4-(4-(2-(2-aminopyridin-3-yl)-5-phenyl-3H-imidazo[4,5-b]pyridin-3-yl)benzyl)piperazin-1-yl)-4-hydroxycyclobut-3-ene-1,2-dione (Example 22, 15 mg, yield: 7.6%) as a light-yellow solid. MS: m/z=558.0 [M+H]⁺. ¹H NMR (400 MHz, Methanol-d₄) δ 8.20 (d, J=8.4 Hz, 1H), 8.05-8.01 (m, 2H), 7.99 (dd, J=5.2, 2.0 Hz, 1H), 7.94 (d, J=8.4 Hz, 1H), 7.61-7.55 (m, 2H), 7.51-7.33 (m, 6H), 6.50 (dd, J=7.6, 4.8 Hz, 1H), 4.00-3.88 (m, 4H), 3.85 (s, 2H), 2.88-2.72 (m, 4H).

### Example 23: 3-(4-(4-(2-(2-Aminopyridin-3-yl)-5-phenyl-3H-imidazo[4,5-b]pyridin-3-yl)benzyl)piperazin-1-yl)-4-(methoxy-d3)cyclobut-3-ene-1,2-dione

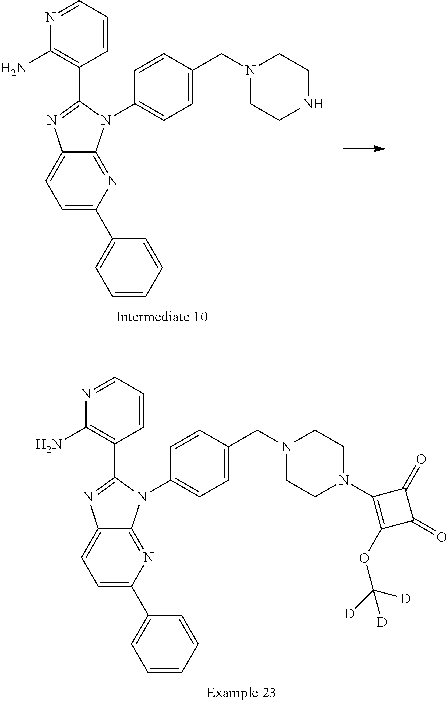 [SMILES: Nc1ncccc1-c1nc2ccc(-c3ccccc3)nc2n1-c1ccc(CN2CCNCC2)cc1 ; [2H]C([2H])([2H])Oc1c(N2CCN(Cc3ccc(-n4c(-c5cccnc5N)nc5ccc(-c6ccccc6)nc54)cc3)CC2)c(=O)c1=O]

Following the general procedure of Example 21, the reaction of Intermediate 10 (200 mg, 433 μmol) with 3,4-bis(trideuteriomethoxy)cyclobut-3-ene-1,2-dione (96.3 mg, 650 μmol) in DCM (4 mL) was carried out. After silica gel flash chromatography (Eluent of 0~5% MeOH in CH₂Cl₂), 3-(4-(4-(2-(2-aminopyridin-3-yl)-5-phenyl-3H-imidazo[4,5-b]pyridin-3-yl)benzyl)piperazin-1-yl)-4-(methoxy-d3)cyclobut-3-ene-1,2-dione (Example 23, 70 mg, yield: 27%) was obtained as a yellow solid. MS: m/z=575.4 [M+H]⁺. ¹H NMR (400 MHz, Dimethylsulfoxide-d₆) δ 8.27 (d, J=8.4 Hz, 1H), 8.04-7.97 (m, 4H), 7.51-7.44 (m, 6H), 7.42-7.33 (m, 1H), 7.15 (dd, J=7.6, 2.0 Hz, 1H), 7.01 (br s, 2H), 6.39 (dd, J=8.0, 4.8 Hz, 1H), 3.85-3.77 (m, 2H), 3.64 (s, 2H), 3.58-3.51 (m, 2H), 2.57-2.53 (m, 4H).

### Example 24: 3-(4-(4-(2-(2-Aminopyridin-3-yl)-5-phenyl-3H-imidazo[4,5-b]pyridin-3-yl)benzyl)piperazin-1-yl)-4-ethoxycyclobut-3-ene-1,2-dione

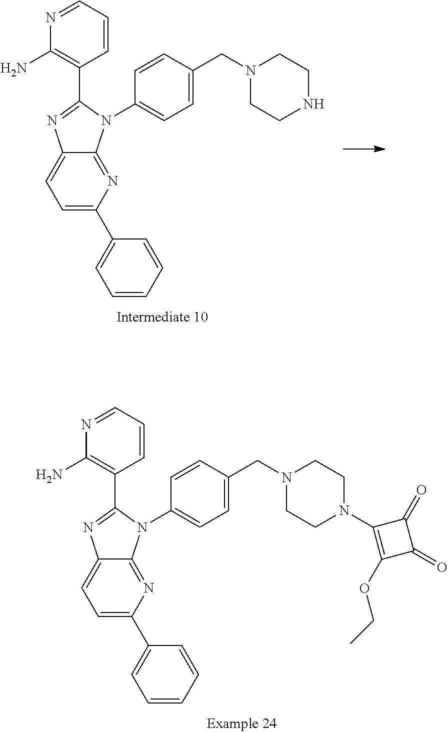 [SMILES: Nc1ncccc1-c1nc2ccc(-c3ccccc3)nc2n1-c1ccc(CN2CCNCC2)cc1 ; CCOc1c(N2CCN(Cc3ccc(-n4c(-c5cccnc5N)nc5ccc(-c6ccccc6)nc54)cc3)CC2)c(=O)c1=O]

Following the general procedure of Example 21, the reaction of Intermediate 10 (180 mg, 390 μmol) with 3,4-diethoxycyclobut-3-ene-1,2-dione (79.6 mg, 468 μmol) was carried out. 3-(4-(4-(2-(2-Aminopyridin-3-yl)-5-phenyl-3H-imidazo[4,5-b]pyridin-3-yl)benzyl)piperazin-1-yl)-4-ethoxycyclobut-3-ene-1,2-dione (Example 24, 152 mg, yield: 62%) was obtained as a brown solid. MS: m/z=586.3 [M+H]⁺. ¹H NMR (400 MHz, Dimethylsulfoxide-d₆) δ 8.27 (d, J=8.4 Hz, 1H), 8.05-7.96 (m, 4H), 7.53-7.37 (m, 7H), 7.16 (dd, J=7.6, 1.6 Hz, 1H), 7.01 (br s, 2H), 6.39 (dd, J=7.6, 4.8 Hz, 1H), 4.66 (q, J=7.2 Hz, 2H), 3.88-3.75 (m, 2H), 3.64 (s, 2H), 3.60-3.53 (m, 2H), 2.56-2.54 (m, 2H), 2.52-2.51 (m, 2H), 1.37 (t, J=7.2 Hz, 3H).

### Example 25: 3-(4-(4-(2-(2-Aminopyridin-3-yl)-5-phenyl-3H-imidazo[4,5-b]pyridin-3-yl)benzyl)piperazin-1-yl)-4-isopropoxycyclobut-3-ene-1,2-dione

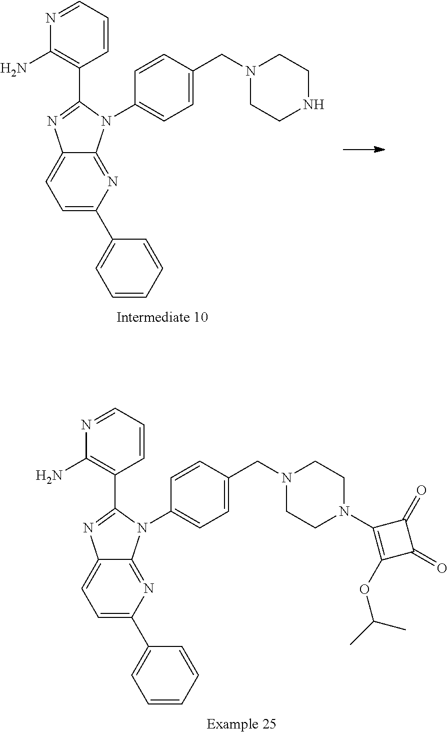 [SMILES: Nc1ncccc1-c1nc2ccc(-c3ccccc3)nc2n1-c1ccc(CN2CCNCC2)cc1 ; CC(C)Oc1c(N2CCN(Cc3ccc(-n4c(-c5cccnc5N)nc5ccc(-c6ccccc6)nc54)cc3)CC2)c(=O)c1=O]

Following the general procedure of Example 21, the reaction of Intermediate 10 (180 mg, 390 μmol) with 3,4-diisopropoxycyclobut-3-ene-1,2-dione (92.8 mg, 468 μmol) was carried out. 3-(4-(4-(2-(2-Aminopyridin-3-yl)-5-phenyl-3H-imidazo[4,5-b]pyridin-3-yl)benzyl)piperazin-1-yl)-4-isopropoxycyclobut-3-ene-1,2-dione (Example 25, 135 mg, yield: 55%) was obtained as a brown solid. MS: m/z=600.3 [M+H]⁺. ¹H NMR (400 MHz, Dimethylsulfoxide-d₆) δ 8.27 (d, J=8.4 Hz, 1H), 8.07-7.94 (m, 4H), 7.53-7.34 (m, 7H), 7.16 (dd, J=7.6, 1.6 Hz, 1H), 7.01 (br s, 2H), 6.39 (dd, J=7.6, 4.8 Hz, 1H), 5.25 (m, 1H), 4.16-4.06 (m, 2H), 3.86-3.75 (m, 2H), 3.64 (s, 2H), 2.58-2.52 (m, 4H), 1.37 (d, J=6.4 Hz, 6H).

### Example 26: 3-(4-(4-(2-(2-aminopyridin-3-yl)-5-phenyl-3H-imidazo[4,5-b]pyridin-3-yl)benzyl)piperazin-1-yl)-4-(cyclopentyloxy)cyclobut-3-ene-1,2-dione

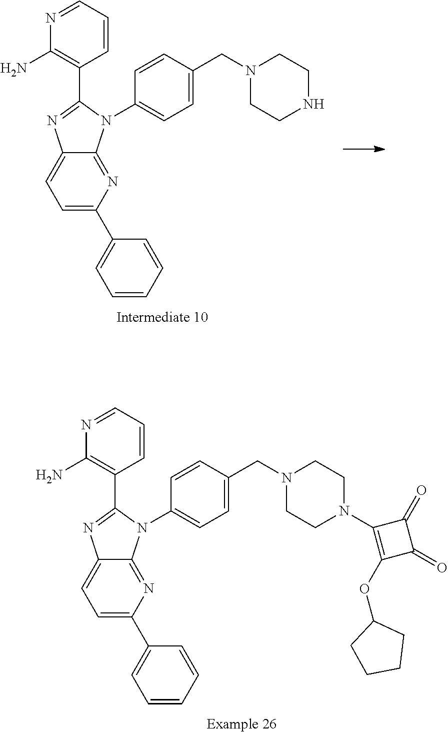 [SMILES: Nc1ncccc1-c1nc2ccc(-c3ccccc3)nc2n1-c1ccc(CN2CCNCC2)cc1 ; Nc1ncccc1-c1nc2ccc(-c3ccccc3)nc2n1-c1ccc(CN2CCN(c3c(OC4CCCC4)c(=O)c3=O)CC2)cc1]

Following the general procedure of Example 21, the reaction of Intermediate 10 (180 mg, 390 μmol) with 3,4-bis(cyclopentyloxy)cyclobut-3-ene-1,2-dione (117 mg, 468 mol) was carried out. 3-(4-(4-(2-(2-Aminopyridin-3-yl)-5-phenyl-3H-imidazo[4,5-b]pyridin-3-yl)benzyl)piperazin-1-yl)-4-(cyclopentyloxy)cyclobut-3-ene-1,2-dione (Example 26, 52.0 mg, yield: 20%) was obtained as a brown solid. MS: m/z=626.4 [M+H]⁺. ¹H NMR (400 MHz, Dimethylsulfoxide-d₆) δ 8.27 (d, J=8.4 Hz, 1H), 8.06-7.96 (m, 4H), 7.51-7.37 (m, 7H), 7.16 (dd, J=7.6, 1.6 Hz, 1H), 7.01 (br s, 2H), 6.39 (dd, J=7.6, 4.8 Hz, 1H), 5.53-5.44 (m, 1H), 3.88-3.72 (m, 2H), 3.65 (s, 2H), 3.59-3.50 (m, 2H), 2.58-2.53 (m, 4H), 1.94-1.80 (m, 4H), 1.72-1.57 (m, 4H).

### Example 27: 3-((1-(4-(2-(2-aminopyridin-3-yl)-5-phenyl-3H-imidazo[4,5-b]pyridin-3-yl)benzyl)piperidin-4-yl)(methyl)amino)-4-methoxycyclobut-3-ene-1,2-dione

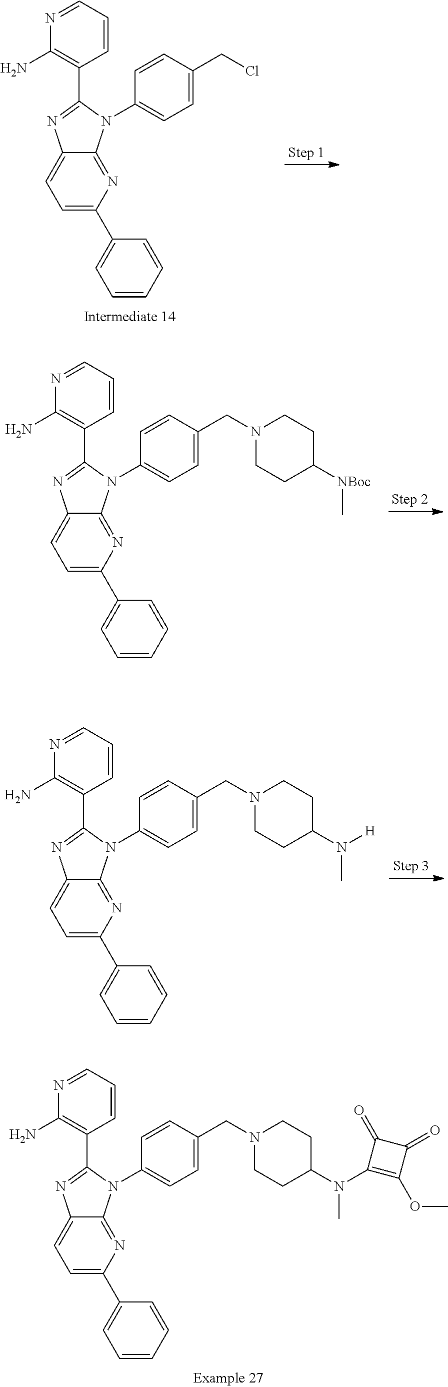 [SMILES: Nc1ncccc1-c1nc2ccc(-c3ccccc3)nc2n1-c1ccc(CCl)cc1 ; CN(C(=O)OC(C)(C)C)C1CCN(Cc2ccc(-n3c(-c4cccnc4N)nc4ccc(-c5ccccc5)nc43)cc2)CC1 ; CNC1CCN(Cc2ccc(-n3c(-c4cccnc4N)nc4ccc(-c5ccccc5)nc43)cc2)CC1 ; COc1c(N(C)C2CCN(Cc3ccc(-n4c(-c5cccnc5N)nc5ccc(-c6ccccc6)nc54)cc3)CC2)c(=O)c1=O]

### Step 1: 3-[3-[4-[[4-(methylamino)-1-piperidyl]methyl]phenyl]-5-phenyl-imidazo[4,5-b]pyridin-2-yl]pyridin-2-amine

To a solution of Intermediate 14 (300 mg, 0.73 mmol) and tert-butyl N-methyl-N-(4-piperidyl)carbamate (312 mg, 1.46 mmol) in DMF (2 mL) were added NaI (18.2 mg, 0.073 mmol) and K₂CO₃ (336 mg, 2.43 mmol). The mixture was stirred at 80° C. for 2 hr. After cooling to 20° C., the reaction mixture was poured into H₂O (3 mL), extracted with EtOAc (10 mL×3). The combined organic layers were dried over anhydrous Na₂SO₄, filtered, and concentrated under reduced pressure. The residue was purified by silica gel flash chromatography (Eluent of 0~7% MeOH in CH₂Cl₂) to give tert-butyl N-[1-[[4-[2-(2-amino-3-pyridyl)-5-phenyl-imidazo[4,5-b]pyridin-3-yl]phenyl]methyl]-4-piperidyl]-N-methyl-carbamate (220 mg, yield: 51%,) as a yellow solid. MS: m/z=590.3 [M+H]⁺.

### Step 2: 3-(3-(4-((4-(Methylamino)piperidin-1-yl)methyl)phenyl)-5-phenyl-3H-imidazo[4,5-b]pyridin-2-yl)pyridin-2-amine

To a solution of tert-butyl N-[1-[[4-[2-(2-amino-3-pyridyl)-5-phenyl-imidazo[4,5-b]pyridin-3-yl]phenyl]methyl]-4-piperidyl]-N-methyl-carbamate (200 mg, 0.339 mmol) in CH₂Cl₂ (2 mL) was added TFA (38.7 mg, 0.339 mmol). The mixture was stirred at 25° C. for 2 hr. The reaction mixture was concentrated to give a residue (185 mg TFA salt, yield: 93%). The crude (50 mg) was diluted with aqueous NaHCO₃ (10 mL) and extracted with CH₂Cl₂ (10 mL×3). The combined organic layers were dried over Na₂SO₄, filtered and concentrated. The residue was purified by prep-TLC (CH₂Cl₂:MeOH=10:1) to give 3-[3-[4-[[4-(methylamino)-1-piperidyl]methyl]phenyl]-5-phenyl-imidazo[4,5-b]pyridin-2-yl]pyridin-2-amine as an off-white solid. MS: m/z=490.2 [M+H]⁺. ¹H NMR (400 MHz, Dimethylsulfoxide-d₆) δ 8.27 (d, J=8.4 Hz, 1H), 8.07-7.94 (m, 4H), 7.53-7.43 (m, 6H), 7.41-7.39 (m, 1H), 7.16 (dd, J=7.8, 1.2 Hz, 1H), 7.02 (br s, 2H), 6.37 (dd, J=7.4, 4.8 Hz, 1H), 3.55 (s, 2H), 3.47-3.37 (m, 1H), 2.80 (br d, J=12.0 Hz, 2H), 2.31 (s, 3H), 2.01 (dd, J=11.4, 9.8 Hz, 2H), 1.85-1.77 (m, 2H), 1.36-1.22 (m, 2H).

### Step 3: 3-((1-(4-(2-(2-Aminopyridin-3-yl)-5-phenyl-3H-imidazo[4,5-b]pyridin-3-yl)benzyl)piperidin-4-yl)(methyl)amino)-4-methoxycyclobut-3-ene-1,2-dione

To a solution of 3-[3-[4-[[4-(methylamino)-1-piperidyl]methyl]phenyl]-5-phenyl-imidazo[4,5-b]pyridin-2-yl]pyridin-2-amine (50 mg, 0.102 mmol) and TEA (51.7 mg, 0.511 mmol) in MeOH (2 mL) was added 3,4-dimethoxycyclobut-3-ene-1,2-dione (17.4 mg, 0.123 mmol). The mixture was stirred at 25° C. for 2 hr. The reaction mixture was concentrated and purified by prep-TLC (DCM:MeOH=10:1) to give 3-((1-(4-(2-(2-Aminopyridin-3-yl)-5-phenyl-3H-imidazo[4,5-b]pyridin-3-yl)benzyl)piperidin-4-yl)(methyl)amino)-4-methoxycyclobut-3-ene-1,2-dione (Example 27, 10.5 mg, yield: 17%, mixture of tautomers) as an off-white solid. MS: m/z=600.3 [M+H]⁺. ¹H NMR (400 MHz, Dimethylsulfoxide-d₆) δ 8.27 (d, J=8.4 Hz, 1H), 8.05-7.93 (m, 4H), 7.50-7.43 (m, 6H), 7.41-7.36 (m, 1H), 7.14 (dd, J=7.2, 1.4 Hz, 1H), 7.02 (br s, 2H), 6.43-6.33 (m, 1H), 4.34-4.28 (m, 3H), 3.72-2.61 (m, 1H), 3.59 (s, 2H), 3.22 (s, 2H), 3.03 (s, 1H), 2.93 (br d, J=10.4 Hz, 2H), 2.07-1.99 (m, 2H), 1.92-1.80 (m, 2H), 1.73-1.62 (m, 2H).

### Example 28: 3-((1-(4-(2-(2-Aminopyridin-3-yl)-5-phenyl-3H-imidazo[4,5-b]pyridin-3-yl)benzoyl)piperidin-4-yl)amino)-4-methoxycyclobut-3-ene-1,2-dione

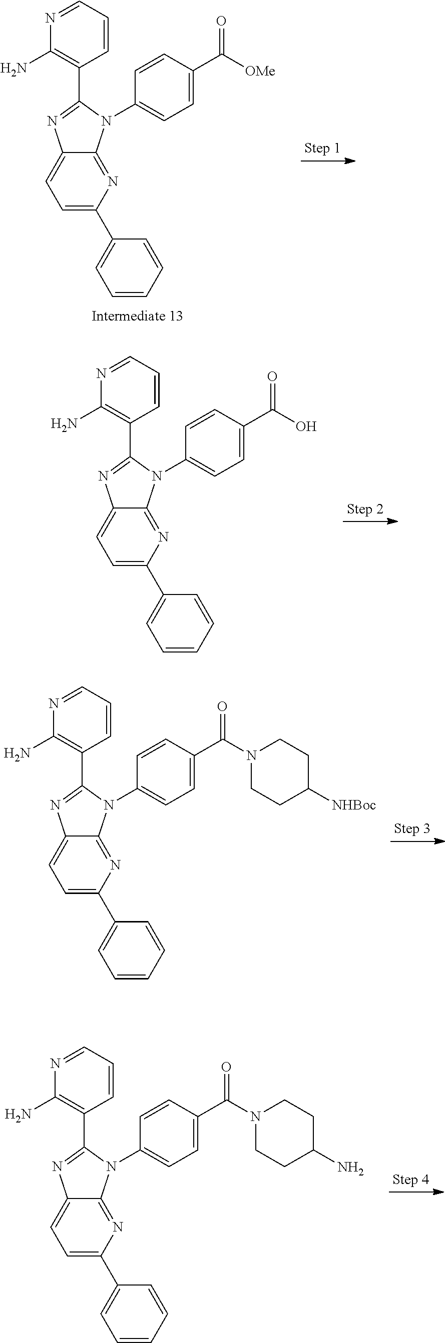 [SMILES: COC(=O)c1ccc(-n2c(-c3cccnc3N)nc3ccc(-c4ccccc4)nc32)cc1 ; Nc1ncccc1-c1nc2ccc(-c3ccccc3)nc2n1-c1ccc(C(=O)O)cc1 ; CC(C)(C)OC(=O)NC1CCN(C(=O)c2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(C(=O)N2CCC(N)CC2)cc1]

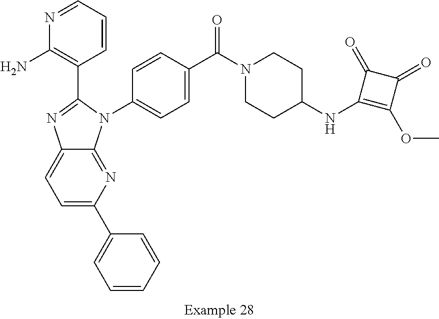 [SMILES: COc1c(NC2CCN(C(=O)c3ccc(-n4c(-c5cccnc5N)nc5ccc(-c6ccccc6)nc54)cc3)CC2)c(=O)c1=O]

### Step 1: 4-(2-(2-Aminopyridin-3-yl)-5-phenyl-3H-imidazo[4,5-b]pyridin-3-yl)benzoic acid

To a solution of Intermediate 13 (500 mg, 1.2 mmol) in H₂O (1 mL) and THE (2 mL) was added LiOH·H₂O (56.8 mg, 2.4 mmol). The mixture was stirred at 25° C. for 16 hr. The reaction mixture was filtered. The filter liquor was concentrated to dryness to give 4-[2-(2-amino-3-pyridyl)-5-phenyl-imidazo[4,5-b]pyridin-3-yl]benzoic acid (485 mg Li salt, yield: 99%) as a yellow solid, which was directly used to next step without further purification. MS: m/z=408.1 [M+H]⁺.

### Step 2: tert-Butyl (1-(4-(2-(2-aminopyridin-3-yl)-5-phenyl-3H-imidazo[4,5-b]pyridin-3-yl)benzoyl)piperidin-4-yl)carbamate

To a solution of 4-[2-(2-amino-3-pyridyl)-5-phenyl-imidazo[4,5-b]pyridin-3-yl]benzoic acid (500 mg, 1.2 mmol), in DMF (5 mL) was added tert-butyl N-(4-piperidyl)carbamate (368.7 mg, 1.8 mmol), DIEA (793 mg, 6.2 mmol) and HATU (700 mg, 1.8 mmol). The resulting mixture was stirred at 25° C. for 16 hr. The reaction mixture was diluted with CH₂Cl₂ (10 mL), the organic phase was separated and washed with H₂O (10 mL×2) and brine (10 mL×2), dried over Na₂SO₄, filtered, and concentrated under reduced pressure to give a residue. The crude product was purified by silica gel flash chromatography (Eluent of 0~5% MeOH in CH₂Cl₂) to give tert-butyl (1-(4-(2-(2-aminopyridin-3-yl)-5-phenyl-3H-imidazo[4,5-b]pyridin-3-yl)benzoyl)piperidin-4-yl)carbamate (645 mg, yield: 89%) as a yellow solid. MS: m/z=590.3 [M+H]r.

### Step 3: (4-Aminopiperidin-1-yl)(4-(2-(2-aminopyridin-3-yl)-5-phenyl-3H-imidazo[4,5-b]pyridin-3-yl)phenyl)methanone

To a solution of tert-butyl (1-(4-(2-(2-aminopyridin-3-yl)-5-phenyl-3H-imidazo[4,5-b]pyridin-3-yl)benzoyl)piperidin-4-yl)carbamate (500 mg, 0.848 mmol) in 1,4-dioxane (5 mL) was added 4M HCl in 1,4-dioxane (1.0 mL) at 15° C. The mixture was stirred at 25° C. for 16 hr. The reaction mixture was filtered. The filter cake was concentrated to dryness. The crude product was triturated with 1,4-dioxane at 25° C. for 30 min to give (4-aminopiperidin-1-yl)(4-(2-(2-aminopyridin-3-yl)-5-phenyl-3H-imidazo[4,5-b]pyridin-3-yl)phenyl)methanone (410 mg HCl salt, yield: 92%) as a yellow solid. MS: m/z=490.1 [M+H]⁺. ¹H NMR (400 MHz, Dimethylsulfoxide-d₆) δ 8.28 (d, J=8.4 Hz, 1H), 8.07-7.99 (m, 4H), 7.60-7.52 (m, 4H), 7.50-7.44 (m, 2H), 7.42-7.38 (m, 1H), 7.22 (dd, J=7.6, 1.6 Hz, 1H), 6.95 (br s, 2H), 6.43 (dd, J=7.6, 4.8 Hz, 1H), 4.45-4.03 (m, 1H), 3.63-3.48 (m, 1H), 3.16-2.94 (m, 2H), 2.88-2.79 (m, 1H), 1.84-1.56 (m, 4H).

### Step 4: 3-((1-(4-(2-(2-Aminopyridin-3-yl)-5-phenyl-3H-imidazo[4,5-b]pyridin-3-yl)benzoyl)piperidin-4-yl)amino)-4-methoxycyclobut-3-ene-1,2-dione

To a solution of (4-amino-1-piperidyl)-[4-[2-(2-amino-3-pyridyl)-5-phenyl-imidazo[4,5-b]pyridin-3-yl]phenyl]methanone (100 mg, 0.204 mmol) and TEA (103 mg, 1.0 mmol) in MeOH (1 mL) was added 3,4-dimethoxycyclobut-3-ene-1,2-dione (34.8 mg, 0.245 mmol). The reaction mixture stirred at 25° C. for 16 hr. The reaction mixture was filtered. The collected solid was washed with MeOH (2 mL×2) and dried in vacuo to give 3-((1-(4-(2-(2-aminopyridin-3-yl)-5-phenyl-3H-imidazo[4,5-b]pyridin-3-yl)benzoyl)piperidin-4-yl)amino)-4-methoxycyclobut-3-ene-1,2-dione (Example 28, 41.2 mg, yield: 32%, 1:1 mixture of tautomers) as a white solid. MS: m/z=600.3 [M+H]⁺. ¹H NMR (400 MHz, Dimethylsulfoxide-d₆) δ 8.92 (br d, J=6.8 Hz, 0.5H), 8.71 (br d, J=7.6 Hz, 0.5H), 8.29 (d, J=8.4 Hz, 1H), 8.09-7.97 (m, 4H), 7.58-7.61 (m, 3H), 7.51-7.44 (m, 2H), 7.43-7.37 (m, 1H), 7.22 (d, J=7.2 Hz, 1H), 6.94 (br s, 2H), 6.44 (dd, J=7.6, 4.2 Hz, 1H), 4.55-4.34 (m, 1H), 4.30 (s, 3H), 4.33-4.27 (m, 3H), 4.22-3.96 (m, 1H), 3.74-3.59 (m, 1H), 3.25-3.08 (m, 2H), 3.07-2.95 (m, 1H), 2.01-1.84 (m, 2H), 1.60-1.45 (m, 2H).

### Example 29: (S)-3-((1-(4-(2-(2-Aminopyridin-3-yl)-5-phenyl-3H-imidazo[4,5-b]pyridin-3-yl)benzyl)piperidin-3-yl)amino)-4-methoxycyclobut-3-ene-1,2-dione

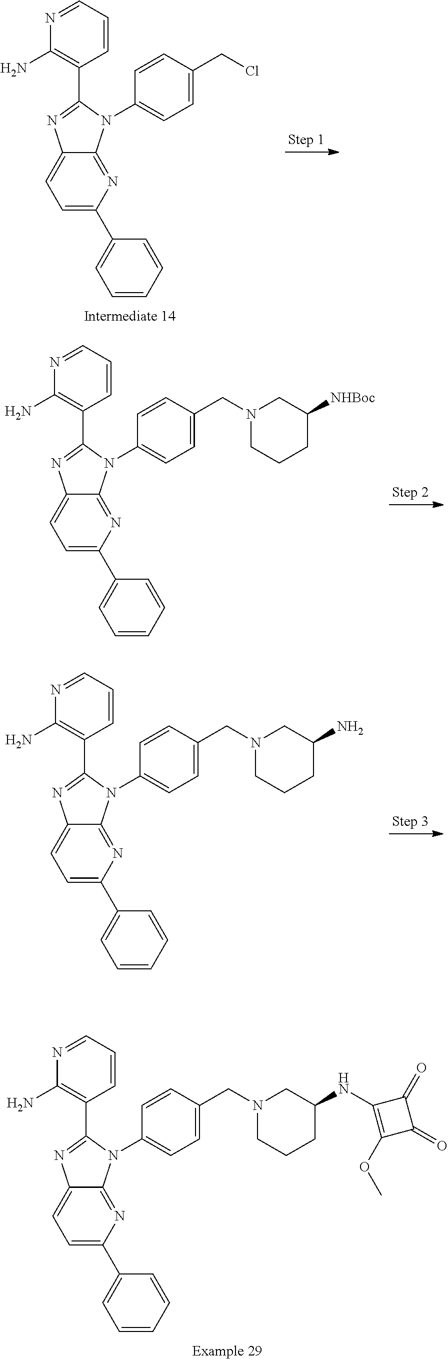 [SMILES: Nc1ncccc1-c1nc2ccc(-c3ccccc3)nc2n1-c1ccc(CCl)cc1 ; CC(C)(C)OC(=O)N[C@H]1CCCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CCC[C@H](N)C2)cc1 ; COc1c(N[C@H]2CCCN(Cc3ccc(-n4c(-c5cccnc5N)nc5ccc(-c6ccccc6)nc54)cc3)C2)c(=O)c1=O]

### Step 1: (S)-tert-Butyl (1-(4-(2-(2-aminopyridin-3-yl)-5-phenyl-3H-imidazo[4,5-b]pyridin-3-yl)benzyl)piperidin-3-yl)carbamate

To a solution of Intermediate 14 (800 mg, 1.9 mmol) in ACN (6 mL) were added tert-butyl N-[(3S)-3-piperidyl]carbamate (427 mg, 2.1 mmol), NaI (29.1 mg, 0.194 mmol) and K₂CO₃ (1.1 g, 7.8 mmol). The mixture was stirred at 80° C. for 2 hr. The reaction mixture was concentrated under reduced pressure. The residue was purified by silica gel flash chromatography (Eluent of 0-50% EtOAc in petroleum ether) to give (S)-tert-butyl (1-(4-(2-(2-aminopyridin-3-yl)-5-phenyl-3H-imidazo[4,5-b]pyridin-3-yl)benzyl)piperidin-3-yl)carbamate (400 mg, yield: 29%) as a brown solid. MS: m/z=576.2 [M+H]⁺.

### Step 2: (S)-3-(3-(4-((3-Aminopiperidin-1-yl)methyl)phenyl)-5-phenyl-3H-imidazo[4,5-b]pyridin-2-yl)pyridin-2-amine

To a solution of tert-butyl N-[(3S)-1-[[4-[2-(2-amino-3-pyridyl)-5-phenyl-imidazo[4,5-b]pyridin-3-yl]phenyl]methyl]-3-piperidyl]carbamate (27 mg, 0.0469 mmol) in CH₂Cl₂ (1 mL) was added TFA (10.7 mg, 0.0938 mmol). The mixture was stirred at 25° C. for 0.5 hr. The mixture was concentrated under reduced pressure to give a crude (26 mg TFA salt, yield: 100%). The crude was purified by prep-HPLC (column: Welch Xtimate C18 150*25 mm*5 μm; mobile phase: [water (HCl)-ACN]; B %: 5%-35%, 8 min) to give (S)-3-(3-(4-((3-aminopiperidin-1-yl)methyl)phenyl)-5-phenyl-3H-imidazo[4,5-b]pyridin-2-yl)pyridin-2-amine (5.3 mg HCl salt) as a light-yellow lyophilized powder. MS: m/z=476.2 [M+H]⁺. 1H NMR (400 MHz, Methanol-d₄) δ 8.32 (d, J=8.4 Hz, 1H), 8.10-7.98 (m, 4H), 7.94 (d, J=8.4 Hz, 2H), 7.89 (dd, J=7.6, 1.2 Hz, 1H), 7.74 (d, J=8.4 Hz, 2H), 7.49-7.34 (m, 3H), 6.93 (dd, J=7.6, 6.4 Hz, 1H), 4.62 (s, 2H), 3.89-3.56 (m, 3H), 3.28-3.12 (m, 2H), 2.26-1.99 (m, 3H), 1.90-1.66 (m, 1H)

### Step 3: (S)-3-((1-(4-(2-(2-Aminopyridin-3-yl)-5-phenyl-3H-imidazo[4,5-b]pyridin-3-yl)benzyl)piperidin-3-yl)amino)-4-methoxycyclobut-3-ene-1,2-dione

To a solution of 3-[3-[4-[[(3S)-3-amino-1-piperidyl]methyl]phenyl]-5-phenyl-imidazo[4,5-b]pyridin-2-yl]pyridin-2-amine (50 mg, 0.105 mmol) in MeOH (2 mL) were added 3,4-dimethoxycyclobut-3-ene-1,2-dione (17.9 mg, 0.126 mmol) and TEA (53.2 mg, 0.525 mmol). The mixture was stirred at 25° C. for 2 hr. The mixture was filtered and the filter cake was concentrated under reduced pressure to give the (S)-3-((1-(4-(2-(2-aminopyridin-3-yl)-5-phenyl-3H-imidazo[4,5-b]pyridin-3-yl)benzyl)piperidin-3-yl)amino)-4-methoxycyclobut-3-ene-1,2-dione (Example 29, 10.4 mg, yield: 16%, 3:2 mixture of tautomers) as a light-yellow solid. MS: m/z=586.3 [M+H]⁺. 1H NMR (400 MHz, Dimethylsulfoxide-d₆) δ 8.82 (d, J=8.0 Hz, 0.6H), 8.62 (d, J=8.0 Hz, 0.4H), 8.27 (d, J=8.4 Hz, 1H), 8.02 (d, J=8.0 Hz, 2H), 8.00-7.96 (m, 2H), 7.50-7.43 (m, 6H), 7.41-7.36 (m, 1H), 7.14 (d, J=7.6 Hz, 1H), 7.03 (br s, 2H), 6.39-6.27 (m, 1H), 4.27 (s, 1.2H), 4.20 (s, 1.8H), 4.07-3.97 (m, 0.4H), 3.68-3.54 (m, 2.6H), 2.91-2.85 (m, 1H), 2.72-2.66 (m, 1H), 2.03-1.92 (m, 2H), 1.90-1.84 (m, 1H), 1.76-1.68 (m, 1H), 1.55-1.45 (m, 1H), 1.37-1.27 (m, 1H).

### Example 30: (S)-3-((1-(4-(2-(2-Aminopyridin-3-yl)-5-phenyl-3H-imidazo[4,5-b]pyridin-3-yl)benzyl)pyrrolidin-3-yl)amino)-4-methoxycyclobut-3-ene-1,2-dione

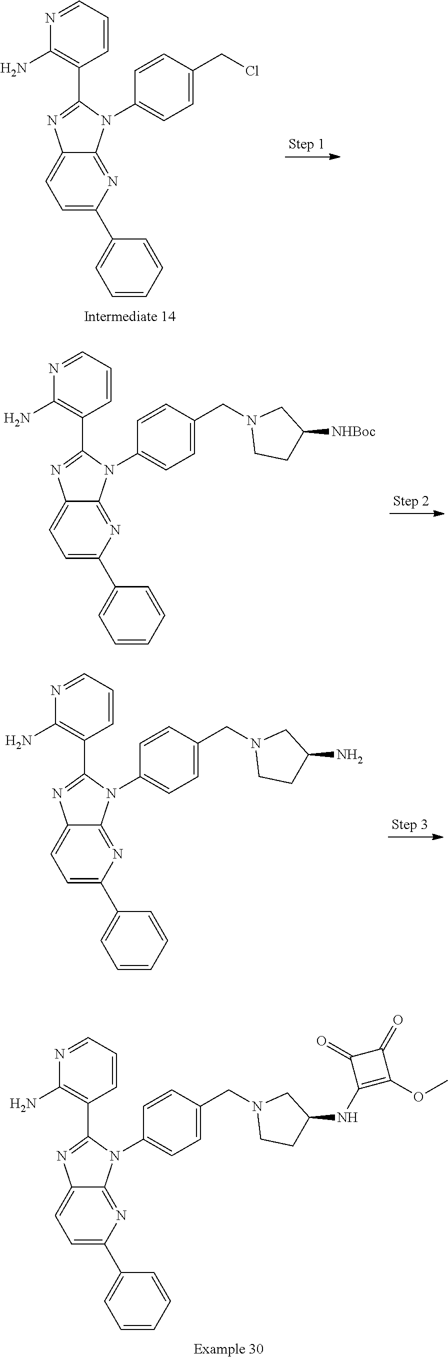

Following the general procedure of Example 29, the reaction was carried out using the corresponding starting material. (S)-3-((1-(4-(2-(2-aminopyridin-3-yl)-5-phenyl-3H-imidazo[4,5-b]pyridin-3-yl)benzyl)pyrrolidin-3-yl)amino)-4-methoxycyclobut-3-ene-1,2-dione (Example 30) was obtained as a light-yellow solid. MS: m/z=572.4 [M+H]⁺. ¹H NMR (400 MHz, Methanol-d₄) δ 8.19 (d, J=8.5 Hz, 1H), 8.05-8.01 (m, 2H), 7.98 (dd, J=4.8, 2.0 Hz, 1H), 7.94 (d, J=8.4 Hz, 1H), 7.55 (d, J=8.4 Hz, 2H), 7.47-7.27 (m, 6H), 6.47 (dd, J=7.6, 4.8, Hz, 1H), 4.36 (s, 3H), 3.78-3.72 (m, 1H), 3.35 (s, 2H), 2.92-2.81 (m, 2H), 2.68-2.56 (m, 2H), 2.40-2.28 (m, 1H), 1.90-1.80 (m, 1H).

### Example 31: 3-(9-(4-(2-(2-Aminopyridin-3-yl)-5-phenyl-3H-imidazo[4,5-b]pyridin-3-yl)benzyl)-2,9-diazaspiro[5.5]undecan-2-yl)-4-methoxycyclobut-3-ene-1,2-dione

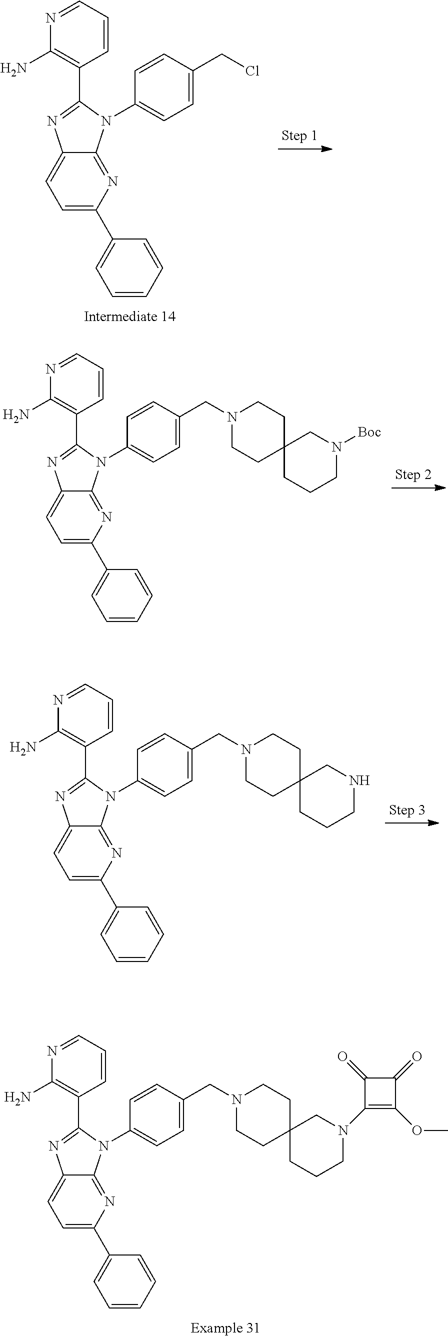 [SMILES: Nc1ncccc1-c1nc2ccc(-c3ccccc3)nc2n1-c1ccc(CCl)cc1 ; CC(C)(C)OC(=O)N1CCCC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CCCNC3)CC2)cc1 ; COc1c(N2CCCC3(CCN(Cc4ccc(-n5c(-c6cccnc6N)nc6ccc(-c7ccccc7)nc65)cc4)CC3)C2)c(=O)c1=O]

### Step 1: tert-Butyl 9-(4-(2-(2-aminopyridin-3-yl)-5-phenyl-3H-imidazo[4,5-b]pyridin-3-yl)benzyl)-2,9-diazaspiro[5.5]undecane-2-carboxylate

To a solution of Intermediate 14 (1.0 g, 2.43 mmol) in DMF (10 mL) were added K₂CO₃ (671 mg, 4.86 mmol) and tert-butyl 2,9-diazaspiro[5.5]undecane-2-carboxylate (679 mg, 2.67 mmol). The mixture was stirred at 25° C. for 48 hr. The reaction mixture was diluted with H₂O (10 mL) at 25° C. and extracted with CH₂Cl₂ (20 mL×3). The combined organic layers were washed with brine (15 mL), dried over Na₂SO₄, filtered, and concentrated under reduced pressure. The crude was purified by silica gel flash chromatography (Eluent of 1-4% MeOH in CH₂Cl₂) to give tert-butyl 9-(4-(2-(2-aminopyridin-3-yl)-5-phenyl-3H-imidazo[4,5-b]pyridin-3-yl)benzyl)-2,9-diazaspiro[5.5]undecane-2-carboxylat (612 mg, yield: 40%) as a yellow solid. MS: m/z=630.5 [M+H]⁺. ¹H NMR (400 MHz, Dimethylsulfoxide-d₆) δ 8.26 (d, J=8.4 Hz, 1H), 8.06-7.94 (m, 4H), 7.48-7.33 (m, 7H), 7.14 (dd, J=7.6, 1.6 Hz, 1H), 7.05 (br s, 2H), 6.35 (dd, J=7.2, 5.2 Hz, 1H), 3.62-3.55 (m, 2H), 3.27 (s, 2H), 3.22-3.15 (m, 2H), 2.49-2.44 (m, 2H), 2.35-2.22 (m, 2H), 1.38-1.34 (m, 8H), 1.38 (s, 9H).

### Step 2: 3-(3-(4-(2,9-Diazaspiro[5.5]undecan-9-ylmethyl)phenyl)-5-phenyl-3H-imidazo[4,5-b]pyridin-2-yl)pyridin-2-amine

A solution of tert-butyl 9-[[4-[2-(2-amino-3-pyridyl)-5-phenyl-imidazo[4,5-b]pyridin-3-yl]phenyl]methyl]-2,9-diazaspiro[5.5]undecane-2-carboxylate (630 mg, 1.0 mmol) in HCl/1,4-dioxane (4M, 6 mL) was stirred at 25° C. for 1 hr. The reaction was concentrated under reduced pressure to give 3-(3-(4-(2,9-diazaspiro[5.5]undecan-9-ylmethyl)phenyl)-5-phenyl-3H-imidazo[4,5-b]pyridin-2-yl)pyridin-2-amine (540 mg, HCl salt) as a yellow solid. MS: m/z=530.4 [M+H]⁺. ¹H NMR (400 MHz, Dimethylsulfoxide-d₆) δ 11.24-10.95 (m, 1H), 9.45-8.99 (m, 2H), 8.52 (br s, 1H), 8.38 (d, J=8.4 Hz, 1H), 8.16 (d, J=6.0 Hz, 1H), 8.12-8.02 (m, 3H), 7.91-7.81 (m, 3H), 7.73-7.62 (m, 2H), 7.52-7.40 (m, 3H), 6.93-6.84 (m, 1H), 4.41 (d, J=5.2 Hz, 2H), 3.21-3.09 (m, 5H), 2.99-2.90 (m, 2H), 2.87-2.75 (m, 1H), 2.13-2.09 (m, 1H), 2.00-1.88 (m, 1H), 1.86-1.73 (m, 2H), 1.72-1.56 (m, 3H), 1.45-1.40 (m, 1H).

### Step 3: 3-(9-(4-(2-(2-Aminopyridin-3-yl)-5-phenyl-3H-imidazo[4,5-b]pyridin-3-yl)benzyl)-2,9-diazaspiro[5.5]undecan-2-yl)-4-methoxycyclobut-3-ene-1,2-dione

To a solution of 3-(3-(4-(2,9-diazaspiro[5.5]undecan-9-ylmethyl)phenyl)-5-phenyl-3H-imidazo[4,5-b]pyridin-2-yl)pyridin-2-amine (50 mg, 94.4 μmol) in MeOH (1 mL) were added TEA (47.8 mg, 472 μmol) and 3,4-dimethoxycyclobut-3-ene-1,2-dione (16.1 mg, 113 μmol). The reaction mixture stirred at 25° C. for 16 hr. The mixture was filtered and the collected solid residue was washed with MeOH (10 mL×2) and dried in vacuo to give 3-(9-(4-(2-(2-aminopyridin-3-yl)-5-phenyl-3H-imidazo[4,5-b]pyridin-3-yl)benzyl)-2,9-diazaspiro[5.5]undecan-2-yl)-4-methoxycyclobut-3-ene-1,2-dione (Example 31, 20.1 mg, yield: 32%) as a brown solid. MS: m/z=640.5 [M+H]⁺. ¹H NMR (400 MHz, Dimethylsulfoxide-d₆) δ 8.26 (d, J=8.4 Hz, 1H), 8.05-7.96 (m, 4H), 7.48-7.36 (m, 7H), 7.15 (d, J=7.6 Hz, 1H), 7.04 (br s, 2H), 6.36 (dd, J=7.6, 4.8 Hz, 1H), 4.30 (s, 3H), 3.76-3.70 (m, 1H), 3.66-3.61 (m, 1H), 3.59-3.54 (m, 2H), 3.49-3.44 (m, 1H), 3.31-3.23 (m, 1H), 2.42-2.28 (m, 4H), 1.66-1.58 (m, 2H), 1.53-1.41 (m, 6H).

### Example 32: 3-((1-(4-(2-(2-Aminopyridin-3-yl)-6-phenyl-3H-imidazo[4,5-b]pyridin-3-yl)benzyl)piperidin-4-yl)(methyl)amino)-4-methoxycyclobut-3-ene-1,2-dione

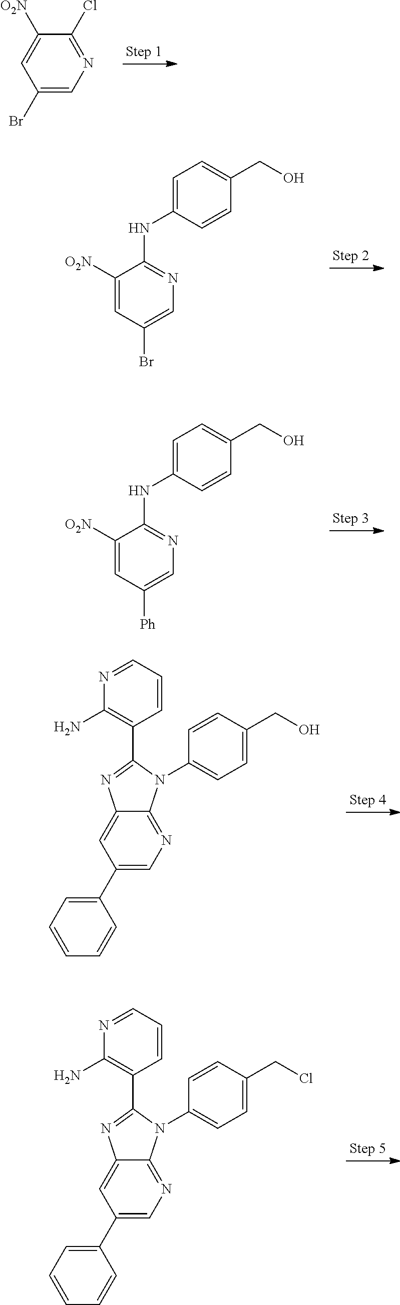

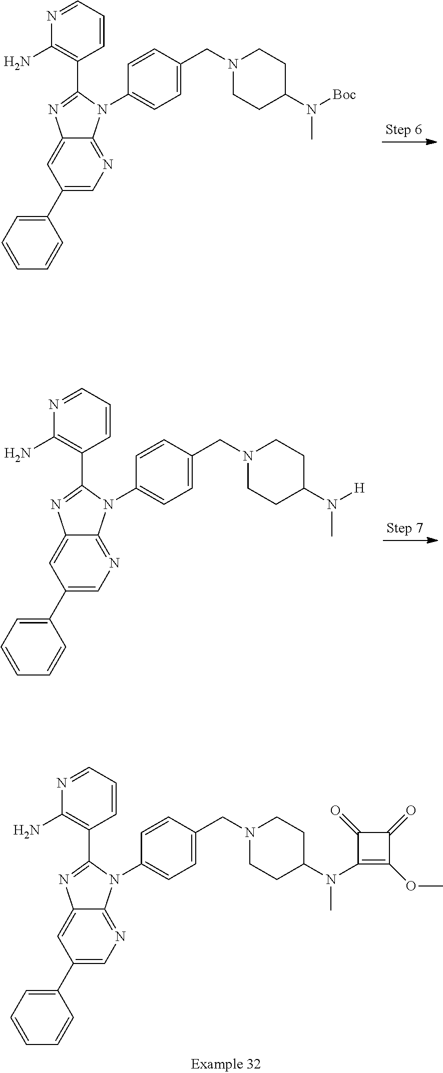 [SMILES: CN(C(=O)OC(C)(C)C)C1CCN(Cc2ccc(-n3c(-c4cccnc4N)nc4cc(-c5ccccc5)cnc43)cc2)CC1 ; CNC1CCN(Cc2ccc(-n3c(-c4cccnc4N)nc4cc(-c5ccccc5)cnc43)cc2)CC1 ; COc1c(N(C)C2CCN(Cc3ccc(-n4c(-c5cccnc5N)nc5cc(-c6ccccc6)cnc54)cc3)CC2)c(=O)c1=O]

### Step 1: (4-((5-Bromo-3-nitropyridin-2-yl)amino)phenyl)methanol

To a solution of 5-bromo-2-chloro-3-nitro-pyridine (10 g, 42 mmol) and (4-aminophenyl)methanol (5.2 g, 42 mmol) in DMSO (100 mL) was added DIEA (16.3 g, 126 mmol). The mixture was stirred at 25° C. for 16 hr. The reaction mixture was poured into H₂O (200 mL) and extracted with EtOAc (300 mL×2). The combined organic layers were washed with brine (200 mL×2), dried over anhydrous Na₂SO₄, filtered, and concentrated. The residue was purified by silica gel flash chromatography (Eluent of 10~50% EtOAc in petroleum ether) to give (4-((5-bromo-3-nitropyridin-2-yl)amino)phenyl)methanol (6.28 g, yield: 46%) as a red solid. MS: m/z=324.0, 325.0 [M+H]⁺. ¹H NMR (400 MHz, Chloroform-d-) δ 10.05 (br s, 1H), 8.65 (d, J=2.4 Hz, 1H), 8.50 (d, J=2.4 Hz, 1H), 7.59 (d, J=8.4 Hz, 2H), 7.40 (d, J=8.4 Hz, 2H), 4.71 (s, 2H).

### Step 2: (4-((3-Nitro-5-phenylpyridin-2-yl)amino)phenyl)methanol

A mixture of [4-[(5-bromo-3-nitro-2-pyridyl)amino]phenyl]methanol (5.4 g, 16.7 mmol), phenylboronic acid (6.1 g, 50 mmol), K₂CO₃ (4.62 g, 33.44 mmol), and Pd(dppf)Cl₂ (1.22 g, 1.67 mmol) in 1,4-dioxane (60 mL) and H₂O (12 mL) was degassed and purged with N₂ three times. The mixture was stirred at 100° C. for 8 hr under N₂ atmosphere. The reaction mixture was poured into H₂O (100 mL) and extracted with CH₂Cl₂ (100 mL×3). The combined organic layers were washed with brine (100 mL×2), dried over anhydrous Na₂SO₄, filtered, and concentrated. The residue was purified by silica gel flash chromatography (Eluent of 0~₅₀% EtOAc in petroleum ether) to give [4-[(3-nitro-5-phenyl-2-pyridyl)amino]phenyl]methanol (4.6 g, yield: 86%) as a red solid. MS: m/z=322.1 [M+H]⁺. ¹H NMR (400 MHz, Chloroform-d) δ 10.15 (s, 1H), 8.78-8.72 (m, 2H), 8.25 (d, J=8.0 Hz 1H), 7.70-7.65 (m, 2H), 7.59-7.56 (m, 2H), 7.51-7.49 (m, 2H), 7.44-7.41 (m, 2H), 4.72 (s, 2H).

### Step 3: (4-(2-(2-Aminopyridin-3-yl)-6-phenyl-3H-imidazo[4,5-b]pyridin-3-yl)phenyl)methanol

To a solution of [4-[(3-nitro-5-phenyl-2-pyridyl)amino]phenyl]methanol (15 g, 46.7 mmol) in DMSO (600 mL) were added Na₂S₂O₄ (16 g, 93 mmol) and 2-aminopyridine-3-carbaldehyde (6.8 g, 56 mmol). The mixture was stirred at 100° C. for 12 hr. After cooling to 25° C., the reaction mixture was diluted with H₂O (200 mL) and extracted with CH₂Cl₂ (200 ml×3). The combined organic layers were washed with brine (200 ml×2), dried over Na₂SO₄, filtered and concentrated under reduced pressure. The residue was purified by silica gel flash chromatography (Eluent of 0~100% EtOAc in petroleum ether) to give [4-[2-(2-amino-3-pyridyl)-6-phenyl-imidazo[4,5-b]pyridin-3-yl]phenyl]methanol (5.15 g crude, yield: 28%) as a yellow solid. MS: m/z=394.0 [M+H]⁺.

### Step 4: 3-(3-(4-(Chloromethyl)phenyl)-6-phenyl-3H-imidazo[4,5-b]pyridin-2-yl)pyridin-2-amine

To a solution of [4-[2-(2-amino-3-pyridyl)-6-phenyl-imidazo[4,5-b]pyridin-3-yl]phenyl]methanol (500 mg, 1.3 mmol) in CH₂Cl₂ (10 mL) was added SOCl₂ (771 mg, 6.5 mmol). The mixture was stirred at 40° C. for 2 hr. The reaction mixture was concentrated under reduced pressure. The residue was purified by silica gel flash chromatography (Eluent of 0-100% EtOAc in petroleum ether) to give 3-[3-[4-(chloromethyl)phenyl]-6-phenyl-imidazo[4,5-b]pyridin-2-yl]pyridin-2-amine (267 mg, yield: 51%) as a yellow solid. MS: m/z=411.8 [M+H]⁺. ¹H NMR (400 MHz, Dimethylsulfoxide-d₆) δ 8.63 (d, J=2.0 Hz, 1H), 8.47 (d, J=2.0 Hz, 1H), 8.01 (dd, J=8.8, 3.2 Hz, 1H), 7.79 (d, J=7.2 Hz, 2H), 7.61 (d, J=8.4 Hz, 2H), 7.55-7.47 (m, 4H), 7.45-7.40 (m, 1H), 7.25 (dd, J=7.6, 1.8 Hz, 1H), 6.96 (br s, 2H), 6.44 (dd, J=7.6, 4.8 Hz, 1H), 4.86 (s, 2H).

### Step 5: tert-Butyl (1-(4-(2-(2-aminopyridin-3-yl)-6-phenyl-3H-imidazo[4,5-b]pyridin-3-yl)benzyl)piperidin-4-yl)(methyl)carbamate

To a solution of 3-[3-[4-(chloromethyl)phenyl]-6-phenyl-imidazo[4,5-b]pyridin-2-yl]pyridin-2-amine (130 mg, 0.315 mmol) and tert-butyl N-methyl-N-(4-piperidyl)carbamate (81 mg, 0.378 mmol) in DMF (2 mL) were added NaI (57 mg, 378 mmol) and K₂CO₃ (130 mg, 946 mmol). The mixture was stirred at 80° C. for 8 hr. After cooling to 20° C., the reaction mixture was poured into H₂O (10 mL), extracted with EtOAc (10 mL×3). The combined organic layers were dried over anhydrous Na₂SO₄, filtered, and concentrated under reduced pressure. The residue was purified by silica gel flash chromatography (Eluent of 0-100% EtOAc in petroleum ether) to give tert-butyl (1-(4-(2-(2-aminopyridin-3-yl)-6-phenyl-3H-imidazo[4,5-b]pyridin-3-yl)benzyl)piperidin-4-yl)(methyl)carbamate (54 mg, yield: 29%) as a yellow solid. MS: m/z=590.5 [M+H]⁺.

### Step 6: 3-(3-(4-((4-(Methylamino)piperidin-1-yl)methyl)phenyl)-6-phenyl-3H-imidazo[4,5-b]pyridin-2-yl)pyridin-2-amine

To a solution of tert-butyl N-[1-[[4-[2-(2-amino-3-pyridyl)-6-phenyl-imidazo[4,5-b]pyridin-3-yl]phenyl]methyl]-4-piperidyl]-N-methyl-carbamate (60 mg, 0.101 mmol) in 1 mL CH₂Cl₂ was added TFA (36 mg, 0.303 mmol). The mixture was stirred at 25° C. for 2 hr. The mixture was concentrated under reduced pressure to give a crude product (55 mg TFA salt, yield: 90%). The crude was triturated with EtOAc (2 mL) at 25° C. for 5 min to give 3-[3-[4-[[4-(methylamino)-1-piperidyl]methyl]phenyl]-6-phenyl-imidazo[4,5-b]pyridin-2-yl]pyridin-2-amine (16.6 mg) as a yellow solid. MS: m/z=490.2 [M+H]⁺. ¹H NMR (400 MHz, Dimethylsulfoxide-d₆) δ 8.63 (d, J=2.0 Hz, 1H), 8.47 (d, J=2.0 Hz, 1H), 8.01 (dd, J=4.8, 1.6 Hz, 1H), 7.79 (d, J=7.2 Hz, 2H), 7.59-7.50 (m, 2H), 7.49-7.35 (m, 5H), 7.20 (dd, J=7.6, 1.6 Hz, 1H), 7.05 (br s, 2H), 6.39 (dd, J=7.6, 4.8 Hz, 1H), 3.57 (s, 2H), ), 2.96-1.84 (m, 3H), 2.54 (s, 3H), 2.06-1.90 (m, 4H), 1.57-1.44 (m, 2H).

### Step 7: 3-((1-(4-(2-(2-Aminopyridin-3-yl)-6-phenyl-3H-imidazo[4,5-b]pyridin-3-yl)benzyl)piperidin-4-yl)(methyl)amino)-4-methoxycyclobut-3-ene-1,2-dione

To a solution of 3-(3-(4-((4-(methylamino)piperidin-1-yl)methyl)phenyl)-6-phenyl-3H-imidazo[4,5-b]pyridin-2-yl)pyridin-2-amine (30 mg, 0.061 mmol) and 3,4-dimethoxycyclobut-3-ene-1,2-dione (10 mg, 73 μmol) in MeOH (2 mL) was added TEA (2.5 mmol). The reaction mixture stirred at 25° C. for 2 hr. The mixture was concentrated under reduced pressure and purified by prep-TLC (DCM:MeOH=10:1) to give 3-((1-(4-(2-(2-Aminopyridin-3-yl)-6-phenyl-3H-imidazo[4,5-b]pyridin-3-yl)benzyl)piperidin-4-yl)(methyl)amino)-4-methoxycyclobut-3-ene-1,2-dione (Example 32, 12 mg, yield: 31%) as a yellow solid. MS: m/z=600.4 [M+H]⁺. ¹H NMR (400 MHz, Methanol-d₄) δ 8.59 (d, J=2.0 Hz, 1H), 8.38 (d, J=2.0 Hz, 1H), 7.98 (dd, J=5.2, 1.8 Hz, 1H), 7.72 (d, J=8.0 Hz 2H), 7.58-7.48 (m, 5H), 7.43-7.36 (m, 2H), 7.29-7.34 (m, 1H), 6.50-6.44 (m, 1H), 3.72-3.63 (m, 1H), 3.35 (s, 3H), 3.30 (s, 3H), 3.12 (s, 2H), 3.09-2.99 (m, 2H), 2.23-2.09 (m, 2H), 2.01-1.93 (m, 2H), 1.79-1.73 (m, 2H).

### Example 33: N-(4-(2-(2-aminopyridin-3-yl)-5-phenyl-3H-imidazo[4,5-b]pyridin-3-yl)benzyl)-3-((2-methoxy-3,4-dioxocyclobut-1-en-1-yl)amino)benzamide

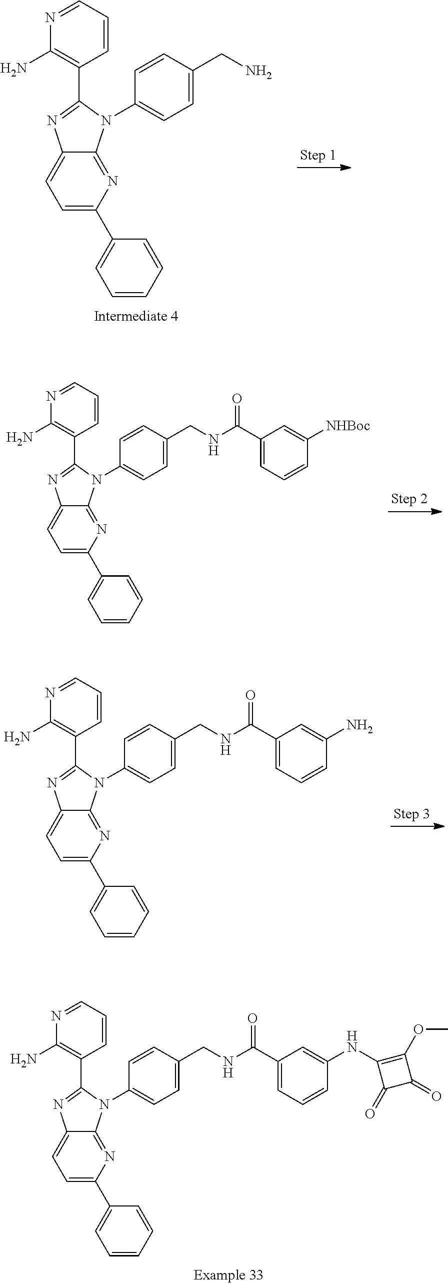 [SMILES: NCc1ccc(-n2c(-c3cccnc3N)nc3ccc(-c4ccccc4)nc32)cc1 ; CC(C)(C)OC(=O)Nc1cccc(C(=O)NCc2ccc(-n3c(-c4cccnc4N)nc4ccc(-c5ccccc5)nc43)cc2)c1 ; Nc1cccc(C(=O)NCc2ccc(-n3c(-c4cccnc4N)nc4ccc(-c5ccccc5)nc43)cc2)c1 ; COc1c(Nc2cccc(C(=O)NCc3ccc(-n4c(-c5cccnc5N)nc5ccc(-c6ccccc6)nc54)cc3)c2)c(=O)c1=O]

### Step 1: tert-Butyl (3-((4-(2-(2-aminopyridin-3-yl)-5-phenyl-3H-imidazo[4,5-b]pyridin-3-yl)benzyl)carbamoyl)phenyl)carbamate

A mixture of Intermediate 4 (500 mg, 1.27 mmol), 3-((tert-butoxycarbonyl)amino)benzoic acid (332 mg, 1.40 mmol), EDCI (366 mg, 1.91 mmol), HOBt (258 mg, 1.91 mmol) and DIEA (659 mg, 5.10 mmol) in DMF (5 mL) was degassed and purged with N₂ three times. The mixture was stirred at 25° C. for 16 hr under N₂ atmosphere. The reaction mixture was quenched with H₂O (10 mL) at 25° C. and extracted with CH₂Cl₂ (20 mL×3). The combined organic layers were washed with brine (20 mL), dried over Na₂SO₄, filtered, and concentrated under reduced pressure. The residue was purified by silica gel flash chromatography (Eluent of 5~25% EtOAc in petroleum ether) to give tert-butyl (3-((4-(2-(2-aminopyridin-3-yl)-5-phenyl-3H-imidazo[4,5-b]pyridin-3-yl)benzyl)carbamoyl)phenyl)carbamate (600 mg, yield: 77%) as an off-white solid. MS: m/z=612.4 [M+H]⁺. ¹H NMR (400 MHz, Dimethylsulfoxide-d₆) δ 9.49 (br s, 1H), 9.06 (t, J=6.0 Hz, 1H), 8.25 (d, J=8.4 Hz, 1H), 8.06-7.95 (m, 4H), 7.58-7.42 (m, 8H), 7.41-7.32 (m, 2H), 7.22 (dd, J=7.6, 2.0 Hz, 1H), 6.94 (br s, 2H), 6.42 (dd, J=7.6, 4.8 Hz, 1H), 4.58 (d, J=6.0 Hz, 2H), 1.48 (s, 9H).

### Step 2: 3-Amino-N-(4-(2-(2-aminopyridin-3-yl)-5-phenyl-3H-imidazo[4,5-b]pyridin-3-yl)benzyl)benzamide

A solution of tert-butyl N-[3-[[4-[2-(2-amino-3-pyridyl)-5-phenyl-imidazo[4,5-b]pyridin-3-yl]phenyl]methylcarbamoyl]phenyl]carbamate (200 mg, 0.327 mmol) in HCl/1,4-dioxane (1 mL) was stirred at 25° C. for 1 hr. The mixture was concentrated under reduced pressure to give a crude product (150 mg HCl salt, yield: 84%). The crude was triturated with MeOH (10 mL×2) and filtrated to give 3-amino-N-(4-(2-(2-aminopyridin-3-yl)-5-phenyl-3H-imidazo[4,5-b]pyridin-3-yl)benzyl)benzamide (60 mg) as a yellow solid. MS: m/z=512.3 [M+H]⁺. ¹H NMR (400 MHz, Methanol-d₄) δ 8.34 (d, J=8.8 Hz, 1H), 8.09-7.91 (m, 7H), 7.73-7.54 (m, 6H), 7.41-7.31 (m, 3H), 6.88 (dd, J=7.6, 7.2 Hz, 1H), 4.75 (s, 2H).

### Step 3: N-(4-(2-(2-Aminopyridin-3-yl)-5-phenyl-3H-imidazo[4,5-b]pyridin-3-yl)benzyl)-3-((2-methoxy-3,4-dioxocyclobut-1-en-1-yl)amino)benzamide

To a solution of 3-amino-N-(4-(2-(2-aminopyridin-3-yl)-5-phenyl-3H-imidazo[4,5-b]pyridin-3-yl)benzyl)benzamide (50 mg, 97.7 μmol) in MeOH (2 mL) were added TEA (0.3 mmol) and 3,4-dimethoxycyclobut-3-ene-1,2-dione (16.7 mg, 117 μmol). The reaction mixture stirred at 25° C. for 16 hr. The mixture was filtered and the collected solid was washed with MeOH (10 mL×2) and dried in vacuo to give N-(4-(2-(2-aminopyridin-3-yl)-5-phenyl-3H-imidazo[4,5-b]pyridin-3-yl)benzyl)-3-((2-methoxy-3,4-dioxocyclobut-1-en-1-yl)amino)benzamide (Example 33, 8.0 mg, yield: 12%) as a brown solid. MS: m/z=622.3 [M+H]⁺. ¹H NMR (400 MHz, Dimethylsulfoxide-d₆) δ 10.90 (br s, 1H), 9.13 (t, J=6.0 Hz, 1H), 8.26 (d, J=8.4 Hz, 1H), 8.04-7.95 (m, 4H), 7.87 (s, 1H), 7.67 (d, J=7.2 Hz, 1H), 7.53-7.36 (m, 9H), 7.22 (dd, J=7.6, 2.0 Hz, 1H), 6.93 (br s, 2H), 6.42 (dd, J=7.6, 4.8 Hz, 1H), 4.61 (d, J=6.0 Hz, 2H), 4.36 (s, 3H).

### Example 34: (R)-3-((1-(4-(2-(2-aminopyridin-3-yl)-5-phenyl-3H-imidazo[4,5-b]pyridin-3-yl)benzyl)piperidin-3-yl)amino)-4-methoxycyclobut-3-ene-1,2-dione

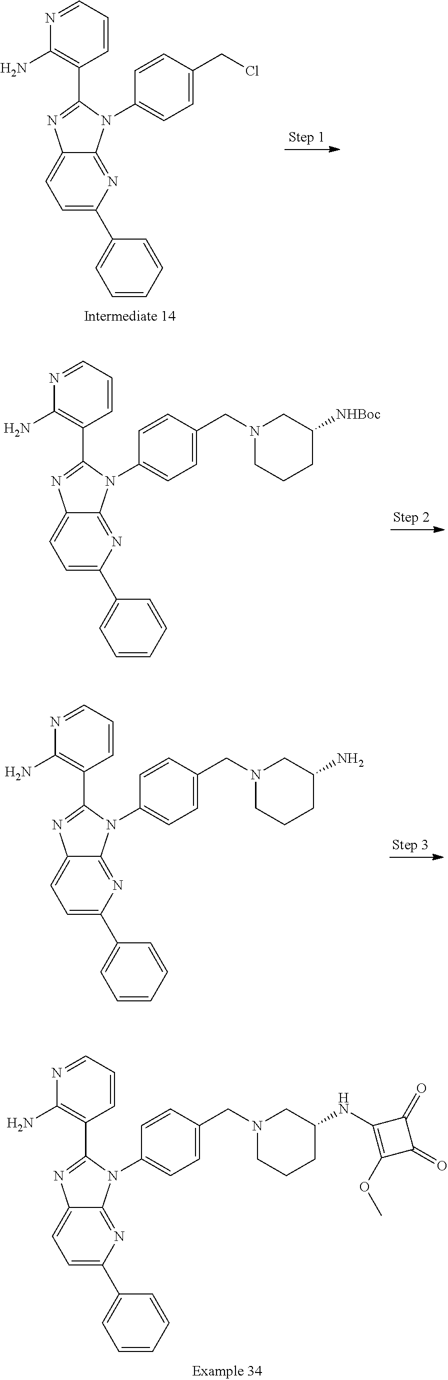 [SMILES: Nc1ncccc1-c1nc2ccc(-c3ccccc3)nc2n1-c1ccc(CCl)cc1 ; CC(C)(C)OC(=O)N[C@@H]1CCCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CCC[C@@H](N)C2)cc1 ; COc1c(N[C@@H]2CCCN(Cc3ccc(-n4c(-c5cccnc5N)nc5ccc(-c6ccccc6)nc54)cc3)C2)c(=O)c1=O]

### Step 1: (R)-tert-Butyl (1-(4-(2-(2-aminopyridin-3-yl)-5-phenyl-3H-imidazo[4,5-b]pyridin-3-yl)benzyl)piperidin-3-yl)carbamate

To a solution of Intermediate 14 (50 mg, 0.121 mmol) in DMF (0.5 mL) were added tert-butyl N-[(3R)-3-piperidyl]carbamate (26.7 mg, 0.133 mmol), NaI (2 mg, 0.0121 mmol) and K₂CO₃ (33.5 mg, 0.242 mmol). The mixture was stirred at 80° C. for 2 hr. The reaction mixture was diluted with H₂O (5 mL) and filtered to give (R)-tert-butyl (1-(4-(2-(2-aminopyridin-3-yl)-5-phenyl-3H-imidazo[4,5-b]pyridin-3-yl)benzyl)piperidin-3-yl)carbamate (60 mg, yield: 85%) as a yellow solid, which was used in the next step without further purification. MS: m/z=576.4 [M+H]⁺.

### Step 2: (R)-3-(3-(4-((3-Aminopiperidin-1-yl)methyl)phenyl)-5-phenyl-3H-imidazo[4,5-b]pyridin-2-yl)pyridin-2-amine

To a solution of (R)-tert-butyl (1-(4-(2-(2-aminopyridin-3-yl)-5-phenyl-3H-imidazo[4,5-b]pyridin-3-yl)benzyl)piperidin-3-yl)carbamate (60 mg, 0.104 mmol) in CH₂Cl₂ (2 mL) was added TFA (770 mg, 6.75 mmol). The mixture was stirred at 20° C. for 2 hr. The reaction mixture was quenched with saturated NaHCO₃ (5 mL) and extracted with CH₂Cl₂ (10 mL×3). The combined organic layers were washed with brine (5 mL), dried over Na₂SO₄, filtered, and concentrated under reduced pressure. The residue was purified by prep-TLC (CH₂Cl₂:MeOH=10:1) to give (R)-3-(3-(4-((3-aminopiperidin-1-yl)methyl)phenyl)-5-phenyl-3H-imidazo[4,5-b]pyridin-2-yl)pyridin-2-amine (6.0 mg, yield: 11.5%) as a light-yellow powder. MS: m/z=476.3 [M+H]⁺. ¹H NMR (400 MHz, Dimethylsulfoxide-d₆) δ 8.27 (d, J=8.0 Hz, 1H), 8.04-7.97 (m, 4H), 7.50-7.44 (m, 6H), 7.42-7.38 (m, 1H), 7.16 (dd, J=7.6, 1.6 Hz, 1H), 7.01 (br s, 2H), 6.39 (dd, J=7.6, 4.8 Hz, 1H), 3.62-3.52 (m, 2H), 2.90-2.85 (m, 1H), 2.79-2.74 (m, 1H), 2.62-2.59 (m, 1H), 2.09-2.00 (m, 1H), 1.95-1.87 (m, 1H), 1.80-1.74 (m, 1H), 1.70-1.64 (m, 1H), 1.53-1.45 (m, 1H), 1.26-1.21 (m, 1H)

### Step 3: (R)-3-((1-(4-(2-(2-Aminopyridin-3-yl)-5-phenyl-3H-imidazo[4,5-b]pyridin-3-yl)benzyl)piperidin-3-yl)amino)-4-methoxycyclobut-3-ene-1,2-dione

To a solution of (R)-3-(3-(4-((3-aminopiperidin-1-yl)methyl)phenyl)-5-phenyl-3H-imidazo[4,5-b]pyridin-2-yl)pyridin-2-amine (100 mg, 0.21 mmol) and 3,4-dimethoxycyclobut-3-ene-1,2-dione (35.8 mg, 0.252 mmol) in MeOH (1 mL) was added TEA (106 mg, 1.05 mmol). The mixture was stirred at 20° C. for 16 hr. The reaction mixture was concentrated under reduced pressure. The residue was purified by prep-TLC (CH₂Cl₂:MeOH=10:1) to give (R)-3-((1-(4-(2-(2-aminopyridin-3-yl)-5-phenyl-3H-imidazo[4,5-b]pyridin-3-yl)benzyl)piperidin-3-yl)amino)-4-methoxycyclobut-3-ene-1,2-dione (Example 34, 9.2 mg, yield: 7.3%, 3:2 mixture of tautomers) as a light-yellow powder. MS: m/z=586.3 [M+H]⁺. ¹H NMR (400 MHz, Dimethylsulfoxide-d₆) δ 8.82 (d, J=8.4 Hz, 0.6H), 8.63 (d, J=8.0 Hz, 0.4H), 8.27 (d, J=8.4 Hz, 1H), 8.06-7.95 (m, 4H), 7.52-7.36 (m, 7H), 7.14 (d, J=7.6 Hz, 1H), 7.03 (br s, 2H), 6.38-6.29 (m, 1H), 4.27 (s, 1.2H), 4.20 (s, 1.8H), 4.16-3.97 (m, 1H), 3.64-3.58 (m, 2H), 3.17 (d, J=4.8 Hz, 1H), 2.91-2.84 (m, 1H), 2.72-2.67 (m, 1H), 2.03-1.98 (m, 2H), 1.90-1.83 (m, 1H), 1.75-1.66 (m, 1H), 1.54-1.46 (m, 1H), 1.36-1.29 (m, 1H).

### Example 35: 3-((7-(4-(2-(2-Aminopyridin-3-yl)-5-phenyl-3H-imidazo[4,5-b]pyridin-3-yl)benzyl)-7-azaspiro[3.5]nonan-2-yl)amino)-4-methoxycyclobut-3-ene-1,2-dione

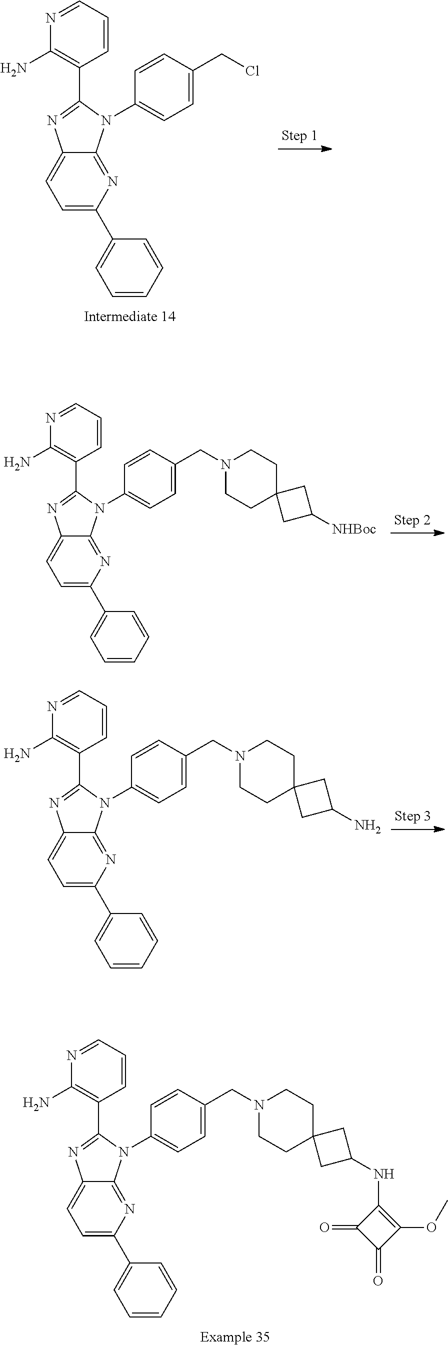

### Step 1: tert-Butyl (7-(4-(2-(2-aminopyridin-3-yl)-5-phenyl-3H-imidazo[4,5-b]pyridin-3-yl)benzyl)-7-azaspiro[3.5]nonan-2-yl)carbamate

To a solution of Intermediate 14 (1 g, 2.43 mmol) and tert-butyl 7-azaspiro[3.5]nonan-2-ylcarbamate (700 mg, 2.92 mmol) in ACN (20 mL) were added NaI (36.4 mg, 0.243 mmol) and K₂CO₃ (671 mg, 4.86 mmol). The mixture was stirred at 80° C. for 2 hr. The reaction mixture was concentrated to give a residue. The residue was purified by silica gel flash chromatography (Eluent of 0~10% MeOH in CH₂Cl₂) to give tert-butyl (7-(4-(2-(2-aminopyridin-3-yl)-5-phenyl-3H-imidazo[4,5-b]pyridin-3-yl)benzyl)-7-azaspiro[3.5]nonan-2-yl)carbamate (480 mg, yield: 32%,) as a light-yellow solid, which was used directly in the next step without further purification. MS: m/z=616.1 [M+H]⁺.

### Step 2: 7-(4-(2-(2-Aminopyridin-3-yl)-5-phenyl-3H-imidazo[4,5-b]pyridin-3-yl)benzyl)-7-azaspiro[3.5]nonan-2-amine

A solution of tert-butyl N-[7-[[4-[2-(2-amino-3-pyridyl)-5-phenyl-imidazo[4,5-b]pyridin-3-yl]phenyl]methyl]-7-azaspiro[3.5]nonan-2-yl]carbamate (450 mg, 0.731 mmol) in HCl/1,4-dioxane (4M, 6 mL) was stirred at 25° C. for 1 hr. The reaction mixture was concentrated. The residue was purified by prep-HPLC (column: Phenomenex C18 150×25 mm×10 μm; mobile phase: [water (NH₄HCO₃)-ACN]; B %: 28%-58%, 8 min) to give 7-[[4-[2-(2-amino-3-pyridyl)-5-phenyl-imidazo[4,5-b]pyridin-3-yl]phenyl]methyl]-7-azaspiro[3.5]nonan-2-amine (370 mg, yield: 91%) as a light-yellow oil. MS: m/z=516.3 [M+H]⁺. ¹H NMR (400 MHz, Dimethylsulfoxide-d₆) δ 8.27 (d, J=8.0 Hz, 1H), 8.06-7.95 (m, 4H), 7.55-7.35 (m, 7H), 7.15 (d, J=7.2 Hz, 1H), 7.01 (br s, 2H), 6.36 (dd, J=7.6, 4.8 Hz, 1H), 3.71-3.58 (m, 1H), 3.52 (s, 2H), 2.41-2.20 (m, 4H), 2.14-2.05 (m, 2H), 1.90-1.80 (m, 2H), 1.67-1.53 (m, 4H).

### Step 3: 3-((7-(4-(2-(2-Aminopyridin-3-yl)-5-phenyl-3H-imidazo[4,5-b]pyridin-3-yl)benzyl)-7-azaspiro[3.5]nonan-2-yl)amino)-4-methoxycyclobut-3-ene-1,2-dione

To a solution of 7-[[4-[2-(2-amino-3-pyridyl)-5-phenyl-imidazo[4,5-b]pyridin-3-yl]phenyl]methyl]-7-azaspiro[3.5]nonan-2-amine (100 mg, 0.181 mmol), 3,4-dimethoxycyclobut-3-ene-1,2-dione (30.9 mg, 0.217 mmol) in MeOH (2 mL) was added TEA (91.6 mg, 0.906 mmol). The mixture was stirred at 25° C. for 2 hr. The reaction mixture was concentrated to give a residue. The residue was purified by prep-TLC (CH₂Cl₂:MeOH=10:1) to give 3-((7-(4-(2-(2-aminopyridin-3-yl)-5-phenyl-3H-imidazo[4,5-b]pyridin-3-yl)benzyl)-7-azaspiro[3.5]nonan-2-yl)amino)-4-methoxycyclobut-3-ene-1,2-dione (Example 35, 11.2 mg, yield: 10%, 1:1 mixture of tautomers) as a light-yellow solid. MS: m/z=626.4 [M+H]⁺. ¹H NMR (400 MHz, Dimethylsulfoxide-d₆) δ 9.18-8.79 (m, 1H), 8.27 (d, J=8.4 Hz, 1H), 8.04-7.97 (m, 4H), 7.48-7.39 (m, 7H), 7.6 (dd, J=7.6, 1.6 Hz, 1H), 7.03 (s, 2H), 6.37 (dd, J=7.6, 4.8 Hz, 1H), 4.53-4.39 (m, 0.5H), 4.29 (s, 3H), 4.10-4.03 (m, 0.5H), 3.52 (s, 2H), 2.38-2.22 (m, 4H), 2.21-2.13 (m, 2H), 1.85-1.77 (m, 2H), 1.55 (br s, 4H).

### Example 36: (S)-3-(4-(4-(2-(2-Aminopyridin-3-yl)-5-phenyl-3H-imidazo[4,5-b]pyridin-3-yl)benzyl)-2-methylpiperazin-1-yl)-4-methoxycyclobut-3-ene-1,2-dione

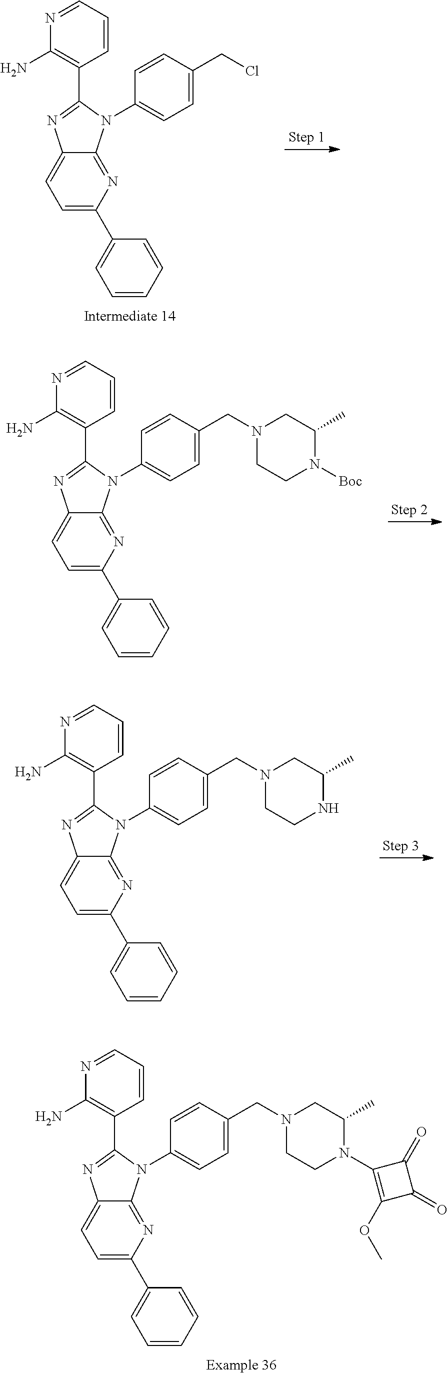 [SMILES: Nc1ncccc1-c1nc2ccc(-c3ccccc3)nc2n1-c1ccc(CCl)cc1 ; C[C@H]1CN(Cc2ccc(-n3c(-c4cccnc4N)nc4ccc(-c5ccccc5)nc43)cc2)CCN1C(=O)OC(C)(C)C ; C[C@H]1CN(Cc2ccc(-n3c(-c4cccnc4N)nc4ccc(-c5ccccc5)nc43)cc2)CCN1 ; COc1c(N2CCN(Cc3ccc(-n4c(-c5cccnc5N)nc5ccc(-c6ccccc6)nc54)cc3)C[C@@H]2C)c(=O)c1=O]

### Step 1: (S)-tert-Butyl 4-(4-(2-(2-aminopyridin-3-yl)-5-phenyl-3H-imidazo[4,5-b]pyridin-3-yl)benzyl)-2-methylpiperazine-1-carboxylate

To a solution of Intermediate 14 (1.0 g, 2.43 mmol) and (S)-tert-butyl 2-methylpiperazine-1-carboxylate (583 mg, 2.91 mmol) in DMF (10 mL) was added K₂CO₃ (671 mg, 4.86 mmol). The mixture was stirred at 25° C. for 24 hr. The reaction mixture was diluted with H₂O (10 mL) at 25° C. and extracted with CH₂Cl₂ (20 mL×3). The combined organic layers were washed with (15 mL), dried over Na₂SO₄, filtered, and concentrated under reduced pressure. The residue was purified by silica gel flash chromatography (Eluent of 0~60% EtOAc in petroleum ether) to give (S)-tert-butyl 4-(4-(2-(2-aminopyridin-3-yl)-5-phenyl-3H-imidazo[4,5-b]pyridin-3-yl)benzyl)-2-methylpiperazine-1-carboxylate (550 mg, yield: 35%) as a yellow solid, which was used in the next step without further purification. MS: m/z=576.1 [M+H]⁺.

### Step 2: (S)-3-(3-(4-((3-Methylpiperazin-1-yl)methyl)phenyl)-5-phenyl-3H-imidazo[4,5-b]pyridin-2-yl)pyridin-2-amine

A solution of (S)-4-(4-(2-(2-aminopyridin-3-yl)-5-phenyl-3H-imidazo[4,5-b]pyridin-3-yl)benzyl)-2-methylpiperazine-1-carboxylate (500 mg, 0.869 mmol) in HCl/1,4-dioxane (4M, 5 mL) was stirred at 25° C. for 1 hr. The reaction mixture was concentrated under reduced pressure to remove 1,4-dioxane. The crude product was triturated with MeOH (2 mL) at 25° C. for 0.5 hr to give (S)-3-(3-(4-((3-methylpiperazin-1-yl)methyl)phenyl)-5-phenyl-3H-imidazo[4,5-b]pyridin-2-yl)pyridin-2-amine (350 mg, yield: 82%) as a yellow solid. MS: m/z=476.2 [M+H]⁺. ¹H NMR (400 MHz, Methanol-d₄) δ 8.32 (d, J=8.4 Hz, 1H), 8.07-7.99 (m, 4H), 7.94-7.84 (m, 3H), 7.73 (d, J=8.4 Hz, 2H), 7.50-7.37 (m, 3H), 6.96-6.88 (m, 1H), 4.57 (s, 1H), 4.60-4.53 (s, 2H), 3.94-3.83 (m, 1H), 3.81-3.69 (m, 3H), 3.68-3.56 (m, 1H), 3.54-3.41 (m, 1H), 3.39-3.31 (m, 1H), 1.45 (d, J=6.8 Hz, 3H).

### Step 3: (S)-3-(4-(4-(2-(2-Aminopyridin-3-yl)-5-phenyl-3H-imidazo[4,5-b]pyridin-3-yl)benzyl)-2-methylpiperazin-1-yl)-4-methoxycyclobut-3-ene-1,2-dione

To a solution of (S)-3-(3-(4-((3-methylpiperazin-1-yl)methyl)phenyl)-5-phenyl-3H-imidazo[4,5-b]pyridin-2-yl)pyridin-2-amine (50 mg, 0.105 mmol) and 3,4-dimethoxycyclobut-3-ene-1,2-dione (18 mg, 0.126 mmol) in MeOH (1 mL) was added TEA (53 mg, 0.526 mmol). The mixture was stirred at 25° C. for 1 hr. The mixture was filtered and concentrated to give (S)-3-(4-(4-(2-(2-aminopyridin-3-yl)-5-phenyl-3H-imidazo[4,5-b]pyridin-3-yl)benzyl)-2-methylpiperazin-1-yl)-4-methoxycyclobut-3-ene-1,2-dione (Example 36, 9.1 mg, yield: 15%) as a yellow solid. MS: m/z=586.4 [M+H]⁺. ¹H NMR (400 MHz, Dimethylsulfoxide-d₆) δ 8.27 (d, J=8.4 Hz, 1H), 8.10-7.90 (m, 4H), 7.53-7.44 (m, 6H), 7.42-7.37 (m, 1H), 7.16 (dd, J=7.6, 1.8 Hz, 1H), 7.02 (br s, 2H), 6.39 (dd, J=7.8, 4.8 Hz, 1H), 4.06 (s, 3H), 4.15-4.02 (m, 1H), 3.73-3.49 (m, 4H), 3.61-3.49 (m, 2H), 2.96-2.83 (m, 1H), 2.73-2.65 (m, 1H), 2.36-2.29 (m, 1H), 2.26-2.13 (m, 1H), 1.36 (d, J=6.8 Hz, 3H).

### Example 37: 3-(7-(4-(2-(2-Aminopyridin-3-yl)-5-phenyl-3H-imidazo[4,5-b]pyridin-3-yl)benzyl)-4,7-diazaspiro[2.5]octan-4-yl)-4-methoxycyclobut-3-ene-1,2-dione

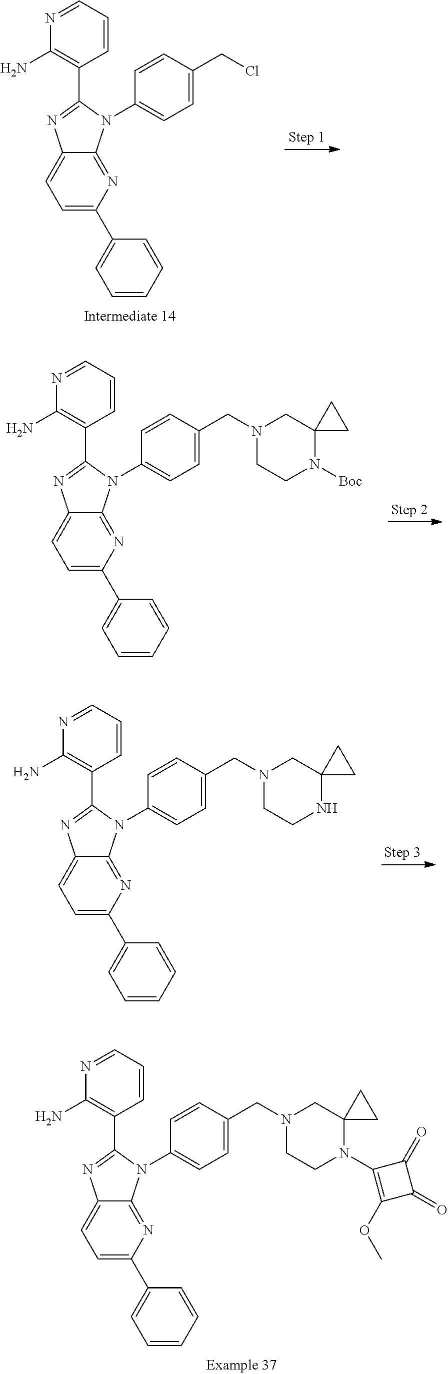 [SMILES: Nc1ncccc1-c1nc2ccc(-c3ccccc3)nc2n1-c1ccc(CCl)cc1 ; CC(C)(C)OC(=O)N1CCN(Cc2ccc(-n3c(-c4cccnc4N)nc4ccc(-c5ccccc5)nc43)cc2)CC12CC2 ; Nc1ncccc1-c1nc2ccc(-c3ccccc3)nc2n1-c1ccc(CN2CCNC3(CC3)C2)cc1 ; COc1c(N2CCN(Cc3ccc(-n4c(-c5cccnc5N)nc5ccc(-c6ccccc6)nc54)cc3)CC23CC3)c(=O)c1=O]

### Step 1: tert-Butyl 7-(4-(2-(2-aminopyridin-3-yl)-5-phenyl-3H-imidazo[4,5-b]pyridin-3-yl)benzyl)-4,7-diazaspiro[2.5]octane-4-carboxylate

To a solution of Intermediate 14 (1.0 g, 2.43 mmol) and tert-butyl 4,7-diazaspiro[2.5]octane-4-carboxylate (618 mg, 2.91 mmol) in DMF (10 mL) was added K₂CO₃ (671 mg, 4.86 mmol). The mixture was stirred at 25° C. for 24 hr. The reaction mixture was diluted with H₂O (10 mL) at 25° C. and extracted with CH₂Cl₂ (20 mL×3). The combined organic layers were washed with brine (15 mL), dried over Na₂SO₄, filtered, and concentrated under reduced pressure. The residue was purified by silica gel flash chromatography (Eluent of 0~60% EtOAc in petroleum ether) to give tert-butyl 7-[[4-[2-(2-amino-3-pyridyl)-5-phenyl-imidazo[4,5-b]pyridin-3-yl]phenyl]methyl]-4,7-diazaspiro[2.5]octane-4-carboxylate (610 mg, yield: 38%) as a yellow solid, which was used in the next step without further purification. MS: m/z=588.1 [M+H]⁺.

### Step 2: 3-(3-(4-(4,7-Diazaspiro[2.5]octan-7-ylmethyl)phenyl)-5-phenyl-3H-imidazo[4,5-b]pyridin-2-yl)pyridin-2-amine

To a solution of tert-butyl 7-[[4-[2-(2-amino-3-pyridyl)-5-phenyl-imidazo[4,5-b]pyridin-3-yl]phenyl]methyl]-4,7-diazaspiro[2.5]octane-4-carboxylate (500 mg, 0.851 mmol) in 1,4-dioxane (3 mL) was added HCl/1,4-dioxane (4M, 5 mL). The mixture was stirred at 25° C. for 1 hr. The reaction mixture was concentrated under reduced pressure to remove 1,4-dioxane. The crude was dissolved in H₂O (5 mL) and extracted with CH₂Cl₂ (5 mL×3). The aqueous phase was added NaHCO₃ and extracted with CH₂Cl₂ (5 mL×3), filtered and concentrated to give 3-[3-[4-(4,7-diazaspiro[2.5]octan-7-ylmethyl)phenyl]-5-phenyl-imidazo[4,5-b]pyridin-2-yl]pyridin-2-amine (350 mg, yield: 81%) as a yellow solid. MS: m/z=488.3 [M+H]⁺. ¹H NMR (400 MHz, Dimethylsulfoxide-d₆) δ 8.26 (d, J=8.4 Hz, 1H), 8.09-7.92 (m, 4H), 7.56-7.33 (m, 7H), 7.14 (dd, J=7.8, 1.8 Hz, 1H), 7.05 (br s, 2H), 6.34 (dd, J=7.6, 4.8 Hz, 1H), 3.52 (s, 2H), 2.76 (t, J=4.8 Hz, 2H), 2.44-2.32 (m, 2H), 2.18 (s, 2H), 0.47-0.38 (m, 2H), 0.35-0.25 (m, 2H).

### Step 3: 3-(7-(4-(2-(2-Aminopyridin-3-yl)-5-phenyl-3H-imidazo[4,5-b]pyridin-3-yl)benzyl)-4,7-diazaspiro[2.5]octan-4-yl)-4-methoxycyclobut-3-ene-1,2-dione

To a solution of 3-[3-[4-(4,7-diazaspiro[2.5]octan-7-ylmethyl)phenyl]-5-phenyl-imidazo[4,5-b]pyridin-2-yl]pyridin-2-amine (50 mg, 0.103 mmol) and 3,4-dimethoxycyclobut-3-ene-1,2-dione (17.5 mg, 0.123 mmol) in MeOH (1 mL) was added TEA (52 mg, 0.513 mmol). The mixture was stirred at 25° C. for 1 hr. The mixture was filtered and concentrated to give 3-(7-(4-(2-(2-Aminopyridin-3-yl)-5-phenyl-3H-imidazo[4,5-b]pyridin-3-yl)benzyl)-4,7-diazaspiro[2.5]octan-4-yl)-4-methoxycyclobut-3-ene-1,2-dione (Example 37, 30 mg, yield: 49%) as a yellow solid. MS: m/z=598.4 [M+H]⁺. 11H NMR (400 MHz, Dimethylsulfoxide-d₆) δ 8.26 (d, J=8.4 Hz, 1H), 8.09-7.92 (m, 4H), 7.56-7.33 (m, 7H), 7.14 (dd, J=8.0, 2.0 Hz, 1H), 7.05 (br s, 2H), 6.34 (dd, J=7.6, 4.8 Hz, 1H), 4.32 (s, 3H), 3.62 (s, 2H), 2.74-2.56 (m, 4H), 2.40 (s, 2H), 1.13-0.97 (m, 2H), 0.76-0.69 (m, 2H).

### Example 38: 3-(4-(4-(2-(2-Aminopyridin-3-yl)-5-phenyl-3H-imidazo[4,5-b]pyridin-3-yl)benzyl)-1,4-diazepan-1-yl)-4-methoxycyclobut-3-ene-1,2-dione

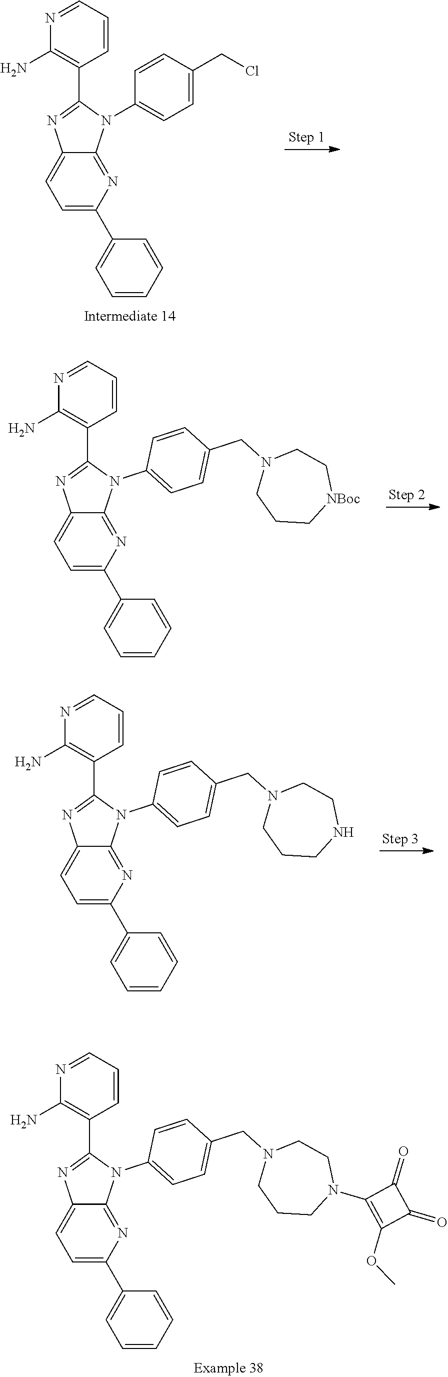

### Step 1: tert-Butyl 4-(4-(2-(2-aminopyridin-3-yl)-5-phenyl-3H-imidazo[4,5-b]pyridin-3-yl)benzyl)-1,4-diazepane-1-carboxylate

To a solution of Intermediate 14 (1 g, 2.43 mmol), tert-butyl 1,4-diazepane-1-carboxylate (584 mg, 2.92 mmol) in ACN (20 mL) were added NaI (36.4 mg, 243 μmol) and K₂CO₃ (671 mg, 4.86 mmol). The mixture was stirred at 80° C. for 2 hr. The reaction mixture was concentrated. The residue was purified by silica gel flash chromatography (Eluent of 0-10% MeOH in CH₂Cl₂) to give tert-butyl 4-[[4-[2-(2-amino-3-pyridyl)-5-phenyl-imidazo[4,5-b]pyridin-3-yl]phenyl]methyl]-1,4-diazepane-1-carboxylate (520 mg, yield: 33%) as a light-yellow solid, which was used in the next step without further purification. MS: m/z=576.4 [M+H]⁺.

### Step 2: 3-(3-(4-((1,4-Diazepan-1-yl)methyl)phenyl)-5-phenyl-3H-imidazo[4,5-b]pyridin-2-yl)pyridin-2-amine

A solution of tert-butyl 4-[[4-[2-(2-amino-3-pyridyl)-5-phenyl-imidazo[4,5-b]pyridin-3-yl]phenyl]methyl]-1,4-diazepane-1-carboxylate (520 mg, 0.903 mmol) in HCl/1,4-dioxane (4M, 10 mL) was stirred at 25 C for 1 hr. The reaction mixture was concentrated to give a crude product (310 mg HCl salt, yield: 67%). The crude (100 mg) was purified by prep-HPLC (column: Phenomenex C18 150×25 mm×10 m; mobile phase: [water (NH₄HCO₃)-ACN]; B %: 28%-58%, 8 min) to give 3-[3-[4-(1,4-diazepan-1-ylmethyl)phenyl]-5-phenyl-imidazo[4,5-b]pyridin-2-yl]pyridin-2-amine (9 mg) as light-yellow solid. MS: m/z=476.3 [M+H]⁺. ¹H NMR (400 MHz, Dimethylsulfoxide-d₆) δ 8.4 (d, J=8.4 Hz, 1H), 8.04-7.97 (m, 4H), 7.52-7.39 (m, 7H), 7.14 (dd, J=7.6, 1.8 Hz, TH), 7.05 (br s, 2H), 6.39-6.34 (m, 1H), 3.72 (s, 2H), 2.82 (t, J=6.4 Hz, 2H), 2.78-2.74 (m, 2H), 2.67 (t, J=6.4 Hz, 2H), 2.66-2.57 (m, 2H), 1.78-1.64 (m, 2H).

### Step 3: 3-(4-(4-(2-(2-Aminopyridin-3-yl)-5-phenyl-3H-imidazo[4,5-b]pyridin-3-yl)benzyl)-1,4-diazepan-1-yl)-4-methoxycyclobut-3-ene-1,2-dione

To a solution of 3-[3-[4-(1,4-diazepan-1-ylmethyl)phenyl]-5-phenyl-imidazo[4,5-b]pyridin-2-yl]pyridin-2-amine (100 mg HCl salt, 195 μmol) and 3,4-dimethoxycyclobut-3-ene-1,2-dione (33.3 mg, 234 μmol) in MeOH (2 mL) was added TEA (98.8 mg, 976 mol). The mixture was stirred at 25 C for 2 hr. The reaction mixture was concentrated. The residue was purified by prep-TLC (CH₂Cl₂:MeOH=10:1) to give 3-(4-(4-(2-(2-aminopyridin-3-yl)-5-phenyl-3H-imidazo[4,5-b]pyridin-3-yl)benzyl)-1,4-diazepan-1-yl)-4-methoxycyclobut-3-ene-1,2-dione (Example 38, 19.1 mg, yield: 16%) as a light yellow solid. MS: m/z=586.3 [M+H]⁺. ¹H NMR (400 MHz, Dimethylsulfoxide-d₆) δ 8.28 (d, J=8.4 Hz, 1H), 7.98 (m, 4H), 7.56-7.36 (m, 7H), 7.14 (dd, J=7.6, 1.6 Hz, 1H), 7.04 (br s, 2H), 6.44-6.33 (m, 1H), 4.29 (s, 3H), 3.90-3.85 (m, 2H), 3.76 (d, J=6.4 Hz, 2H), 3.68-3.57 (m, 2H), 2.76 (dd, J=9.6, 5.2 Hz, 2H), 2.70-2.66 (m, 2H), 1.89-1.78 (m, 2H).

### Example 39: 3-((1-(4-(2-(2-Aminopyridin-3-yl)-5-phenyl-3H-imidazo[4,5-b]pyridin-3-yl)benzyl)piperidin-4-yl)amino)-4-(ethylthio)cyclobut-3-ene-1,2-dione

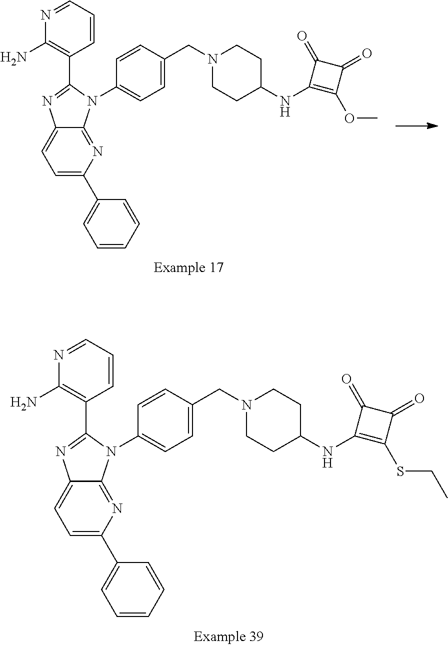 [SMILES: COc1c(NC2CCN(Cc3ccc(-n4c(-c5cccnc5N)nc5ccc(-c6ccccc6)nc54)cc3)CC2)c(=O)c1=O ; CCSc1c(NC2CCN(Cc3ccc(-n4c(-c5cccnc5N)nc5ccc(-c6ccccc6)nc54)cc3)CC2)c(=O)c1=O]

To a solution of Example 17 (200 mg, 0.341 mmol) in CH₂Cl₂ (1 mL) were added TEA (104 mg, 1.02 mmol) and ethanethiol (2.23 g, 35.9 mmol). The mixture was stirred at 25° C. for 24 hr. The reaction was concentrated under reduced pressure to give a residue. The crude product was purified by silica gel flash chromatography (Eluent of 50-80% EtOAc in petroleum ether) to give 3-((1-(4-(2-(2-aminopyridin-3-yl)-5-phenyl-3H-imidazo[4,5-b]pyridin-3-yl)benzyl)piperidin-4-yl)amino)-4-(ethylthio)cyclobut-3-ene-1,2-dione (Example 39, 39.4 mg, yield: 12%) as a yellow solid. MS: m/z=616.4 [M+H]⁺. ¹H NMR (400 MHz, Dimethylsulfoxide-d₆) δ 9.27 (d, J=8.4 Hz, 0.5H), 8.86 (d, J=8.4 Hz, 0.5H), 8.27 (d, J=8.4 Hz, 1H), 8.07-7.92 (m, 4H), 7.50-7.36 (m, 7H), 7.15 (d, J=7.6 Hz, 1H), 7.04 (br s, 2H), 6.41-6.35 (m, 1H), 3.58 (s, 2H), 3.45-3.33 (m, 3H), 2.93-2.82 (m, 2H), 2.10-1.99 (m, 2H), 1.90-1.81 (m, 2H), 1.69-1.54 (m, 2H), 1.36 (t, J=7.2 Hz, 3H).

### Example 40: 3-(6-(4-(2-(2-Aminopyridin-3-yl)-5-phenyl-3H-imidazo[4,5-b]pyridin-3-yl)benzyl)-2,6-diazaspiro[3.3]heptan-2-yl)-4-ethoxycyclobut-3-ene-1,2-dione

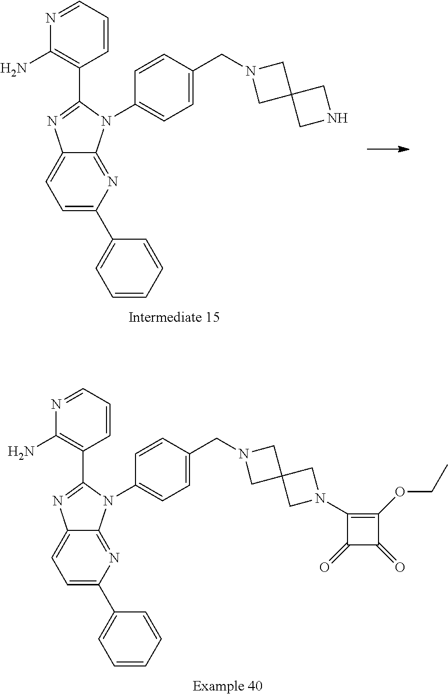 [SMILES: Nc1ncccc1-c1nc2ccc(-c3ccccc3)nc2n1-c1ccc(CN2CC3(CNC3)C2)cc1 ; CCOc1c(N2CC3(CN(Cc4ccc(-n5c(-c6cccnc6N)nc6ccc(-c7ccccc7)nc65)cc4)C3)C2)c(=O)c1=O]

To a solution of Intermediate 15 (100 mg, 211 μmol) in MeOH (2 mL) were added TEA (64 mg, 633 μmol) and 3,4-diethoxycyclobut-3-ene-1,2-dione (36 mg, 211 μmol). The reaction mixture was stirred at 25° C. for 2 hr. The reaction mixture was quenched with saturated NaHCO₃ (aq) (10 mL) at 25° C. and extracted with CH₂Cl₂ (50 mL×2). The combined organic layers were washed with brine (50 mL), dried over Na₂SO₄, filtered, and concentrated under reduced pressure. After purified by silica gel flash chromatography (MeOH in CH₂Cl₂=0% to 8%), 3-(6-(4-(2-(2-aminopyridin-3-yl)-5-phenyl-3H-imidazo[4,5-b]pyridin-3-yl)benzyl)-2,6-diazaspiro[3.3]heptan-2-yl)-4-ethoxycyclobut-3-ene-1,2-dione (Example 40, 42.9 mg, yield: 33%) was obtained as a yellow solid. MS: m/z=598.3 [M+H]⁺. ¹H NMR (400 MHz, Methanol-d₄) δ 8.19 (d, J=8.4 Hz, 1H), 8.03 (d, J=7.2 Hz, 2H), 7.98 (dd, J=5.2, 1.6 Hz, 1H), 7.94 (d, J=8.8 Hz, 1H), 7.49-7.35 (m, 7H), 7.32 (dd, J=7.6, 2.0 Hz, 1H), 6.48 (dd, J=7.6, 5.2 Hz, 1H), 4.65 (q, J=7.2 Hz, 2H), 4.60-4.51 (m, 4H), 3.71 (s, 2H), 3.52 (s, 4H), 1.42 (t, J=7.2 Hz, 3H).

### Example 41: 3-(6-(4-(2-(2-Aminopyridin-3-yl)-5-phenyl-3H-imidazo[4,5-b]pyridin-3-yl)benzyl)-2,6-diazaspiro[3.3]heptan-2-yl)-4-methoxycyclobut-3-ene-1,2-dione

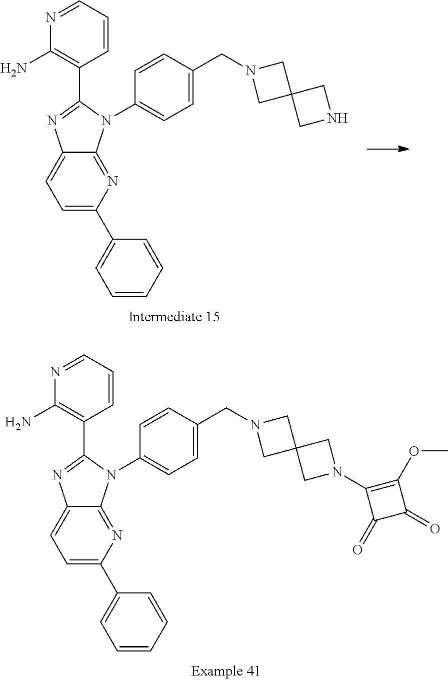 [SMILES: Nc1ncccc1-c1nc2ccc(-c3ccccc3)nc2n1-c1ccc(CN2CC3(CNC3)C2)cc1 ; COc1c(N2CC3(CN(Cc4ccc(-n5c(-c6cccnc6N)nc6ccc(-c7ccccc7)nc65)cc4)C3)C2)c(=O)c1=O]

To a solution of Intermediate 15 (50 mg, 105 μmol) in MeOH (2 mL) were added TEA (23.5 mg, 232 mol) and 3,4-dimethoxycyclobut-3-ene-1,2-dione (15 mg, 105 μmol). The reaction mixture was stirred at 25° C. for 2 hr. The reaction mixture was quenched with NaHCO₃ (aq) (10 mL) at 25° C. and extracted with CH₂Cl₂ (50 mL×2). The combined organic layers were washed with brine (50 mL), dried over Na₂SO₄, filtered and concentrated under reduced pressure. The residue was purified by silica gel flash chromatography (Eluent of 0-8% MeOH in CH₂Cl₂) to give 3-(6-(4-(2-(2-aminopyridin-3-yl)-5-phenyl-3H-imidazo[4,5-b]pyridin-3-yl)benzyl)-2,6-diazaspiro[3.3]heptan-2-yl)-4-methoxycyclobut-3-ene-1,2-dione (Example 41, 14.3 mg, yield: 22%) as a yellow solid. MS: m/z=584.3 [M+H]⁺. ¹H NMR (400 MHz, Dimethylsulfoxide-d₆) δ 8.26 (d, J=8.4 Hz, 1H), 8.05-7.96 (m, 4H), 7.49-7.38 (m, 7H), 7.18-7.11 (m, 1H), 6.99 (br s, 2H), 6.44-6.36 (m, 1H), 4.57-4.38 (m, 4H), 4.28-4.18 (m, 3H), 3.61 (s, 2H), 3.40-3.37 (m, 4H).

### Example 42: 3-(2-(4-(2-(2-Aminopyridin-3-yl)-5-phenyl-3H-imidazo[4,5-b]pyridin-3-yl)benzyl)-2,6-diazaspiro[3.4]octan-6-yl)-4-methoxycyclobut-3-ene-1,2-dione

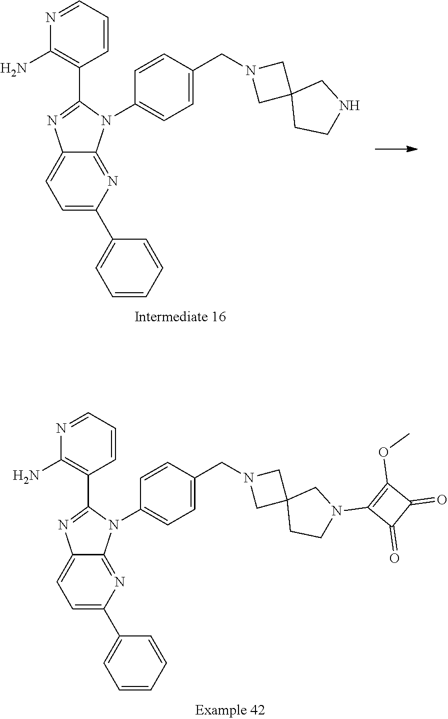 [SMILES: Nc1ncccc1-c1nc2ccc(-c3ccccc3)nc2n1-c1ccc(CN2CC3(CCNC3)C2)cc1 ; COc1c(N2CCC3(CN(Cc4ccc(-n5c(-c6cccnc6N)nc6ccc(-c7ccccc7)nc65)cc4)C3)C2)c(=O)c1=O]

To a solution of Intermediate 16 (100 mg, 205 μmol) in MeOH (2 mL) were added TEA (45 mg, 451 μmol) and 3,4-dimethoxycyclobut-3-ene-1,2-dione (29 mg, 205 μmol). The reaction mixture was stirred at 25° C. for 2 hr. The reaction mixture was quenched with NaHCO₃ (aq) (10 mL) at 25° C. and extracted with CH₂Cl₂ (50 mL×2). The combined organic layers were washed with brine (50 mL), dried over Na₂SO₄, filtered and concentrated under reduced pressure. The residue was purified by silica gel flash chromatography (Eluent of 0-8% MeOH in CH₂Cl₂) to give 3-(2-(4-(2-(2-aminopyridin-3-yl)-5-phenyl-3H-imidazo[4,5-b]pyridin-3-yl)benzyl)-2,6-diazaspiro[3.4]octan-6-yl)-4-methoxycyclobut-3-ene-1,2-dione (Example 42, 12.9 mg, yield: 10%) as a yellow solid. MS: m/z=598.4 [M+H]⁺. ¹H NMR (400 MHz, Dimethylsulfoxide-d₆) δ 8.27 (d, J=8.4 Hz, 1H), 8.07-7.94 (m, 4H), 7.51-7.37 (m, 7H), 7.16 (dd, J=8.0, 2.0 Hz, 1H), 6.99 (br s, 2H), 6.40 (dd, J=8.0, 5.2 Hz, 1H), 4.29-4.25 (m, 3H), 3.85-3.68 (m, 4H), 3.23-3.17 (m, 4H), 2.14-2.06 (m, 2H), 1.26-1.22 (m, 2H).

### Example 43: N-(4-(2-(2-Aminopyridin-3-yl)-5-phenyl-3H-imidazo[4,5-b]pyridin-3-yl)benzyl)-3-((2-hydroxy-3,4-dioxocyclobut-1-en-1-yl)amino)benzamide

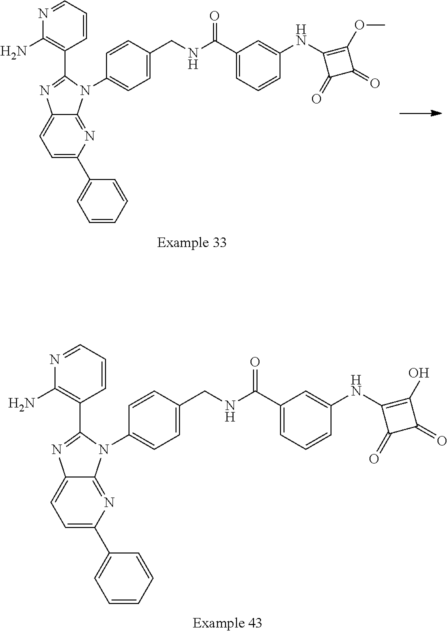 [SMILES: COc1c(Nc2cccc(C(=O)NCc3ccc(-n4c(-c5cccnc5N)nc5ccc(-c6ccccc6)nc54)cc3)c2)c(=O)c1=O ; Nc1ncccc1-c1nc2ccc(-c3ccccc3)nc2n1-c1ccc(CNC(=O)c2cccc(Nc3c(O)c(=O)c3=O)c2)cc1]

To solution of Example 33 (150 mg, 241 μmol) in THE (1 mL) and H₂O (1 mL) was added K₂CO₃ (100 mg, 724 μmol). The mixture was stirred at 25° C. for 0.5 hr. The reaction mixture was diluted with CH₂Cl₂ (20 mL) and washed with water (20 ml×2) and brine (20 ml×2). The organic layer was dried over Na₂SO₄, filtered and concentrated under reduced pressure. After purified by prep-HPLC (column: Welch Xtimate C₁₈ 150×25 mm×10 μm; mobile phase: [water (NH₄HCO₃)-ACN]; gradient: 10%-40%, 10 min), N-(4-(2-(2-aminopyridin-3-yl)-5-phenyl-3H-imidazo[4,5-b]pyridin-3-yl)benzyl)-3-((2-hydroxy-3,4-dioxocyclobut-1-en-1-yl)amino)benzamide (Example 43, 23.4 mg, yield: 15%) was obtained as a yellow solid. MS: m/z=608.3. [M+H]⁺. ¹H NMR (400 MHz, Dimethylsulfoxide-d₆) δ 9.41 (s, 1H), 8.93 (t, J=6.0 Hz, 1H), 8.26 (d, J=8.4 Hz, 1H), 8.05-7.94 (m, 6H), 7.51-7.37 (m, 7H), 7.31-7.26 (m, 2H), 7.22 (dd, J=7.6, 1.6 Hz, 1H), 6.95 (br s, 2H), 6.43 (dd, J=7.6, 4.8 Hz, 1H), 4.58 (d, J=5.6 Hz, 2H).

### Example 44 and 45: 3-((1-(4-(2-(2-Aminopyridin-3-yl)-5-phenyl-3H-imidazo[4,5-b]pyridin-3-yl)benzyl)piperidin-4-yl)amino)-4-mercaptocyclobut-3-ene-1,2-dione and 3-((1-(4-(2-(2-aminopyridin-3-yl)-5-phenyl-3H-imidazo[4,5-b]pyridin-3-yl)benzyl)piperidin-4-yl)amino)-4-(methylthio)cyclobut-3-ene-1,2-dione

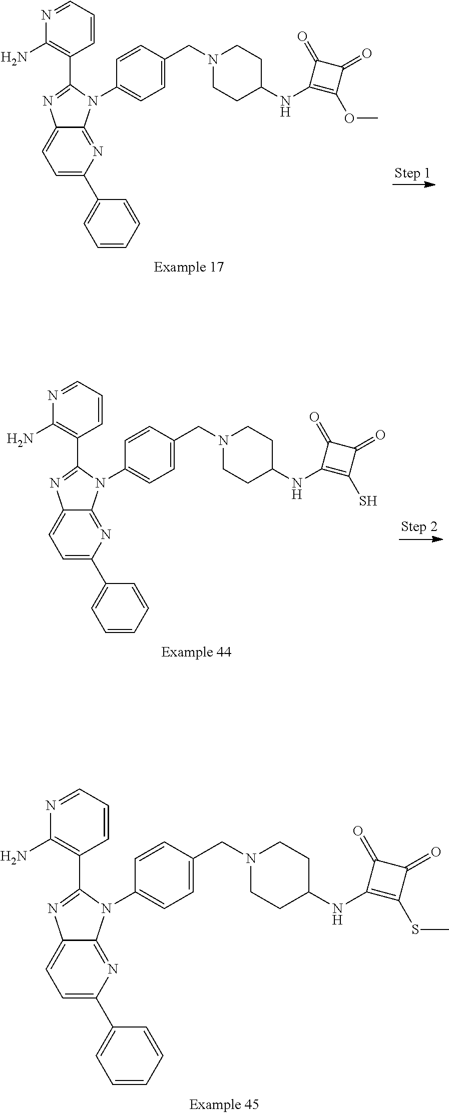 [SMILES: COc1c(NC2CCN(Cc3ccc(-n4c(-c5cccnc5N)nc5ccc(-c6ccccc6)nc54)cc3)CC2)c(=O)c1=O ; Nc1ncccc1-c1nc2ccc(-c3ccccc3)nc2n1-c1ccc(CN2CCC(Nc3c(S)c(=O)c3=O)CC2)cc1 ; CSc1c(NC2CCN(Cc3ccc(-n4c(-c5cccnc5N)nc5ccc(-c6ccccc6)nc54)cc3)CC2)c(=O)c1=O]

### Step 1: 3-((1-(4-(2-(2-Aminopyridin-3-yl)-5-phenyl-3H-imidazo[4,5-b]pyridin-3-yl)benzyl)piperidin-4-yl)amino)-4-mercaptocyclobut-3-ene-1,2-dione

To a solution of Example 17 (100 mg, 171 mol) in EtOH (2 mL) was added NaSH (28.7 mg, 512 mol). The mixture was stirred at 25° C. for 16 hr. The reaction was concentrated under reduced pressure. After purified by prep-HPLC (column: Phenomenex C18 150×25 mm×10 μm; mobile phase: [water (NH₄HCO₃)-ACN]; B %: 20%-50%, 14 min), 3-((1-(4-(2-(2-aminopyridin-3-yl)-5-phenyl-3H-imidazo[4,5-b]pyridin-3-yl)benzyl)piperidin-4-yl)amino)-4-mercaptocyclobut-3-ene-1,2-dione (Example 44, 6.7 mg, yield: 6.5%) was obtained as a yellow lyophilized powder. MS: m/z=588.2 [M+H]⁺. ¹H NMR (400 MHz, Methanol-d₄) δ 8.22 (d, J=8.4 Hz, 1H), 8.06-7.98 (m, 3H), 7.95 (d, J=8.4 Hz, 1H), 7.67 (d, J=8.0 Hz, 2H), 7.62 (d, J=8.0 Hz, 2H), 7.47-7.36 (m, 4H), 6.52 (dd, J=7.6, 4.8 Hz, 1H), 4.59-4.53 (m, 1H), 4.31-4.17 (m, 2H), 3.50-3.38 (m, 2H), 3.12-2.93 (m, 2H), 2.32-2.15 (m, 2H), 2.01-1.79 (m, 2H).

### Step 2: 3-((1-(4-(2-(2-Aminopyridin-3-yl)-5-phenyl-3H-imidazo[4,5-b]pyridin-3-yl)benzyl)piperidin-4-yl)amino)-4-(methylthio)cyclobut-3-ene-1,2-dione

To a solution of Example 44 (150 mg, 255 μmol) in EtOH (2 mL) was added CH₃I (72.5 mg, 510 mol) at 0° C. The mixture was stirred at 0° C. for 1 hr. The mixture was filtrated and washed with MeOH (10 mL×2). The filtrate was concentrated under reduced pressure. After purified by prep-HPLC (column: Waters xbridge 150×25 mm×10 μm; mobile phase: [water (NH₄HCO₃)-ACN]; B %: 38%-68%, 8 min), 3-((1-(4-(2-(2-aminopyridin-3-yl)-5-phenyl-3H-imidazo[4,5-b]pyridin-3-yl)benzyl)piperidin-4-yl)amino)-4-(methylthio)cyclobut-3-ene-1,2-dione (Example 45, 15.5 mg, yield: 10%, 1:1 mixture of tautomers) was obtained as a yellow lyophilized powder. MS: m/z=602.4 [M+H]⁺. ¹H NMR (400 MHz, Dimethylsulfoxide-d₆) δ 9.25 (d, J=8.0 Hz, 0.5H), 8.85 (d, J=8.0 Hz, 0.5H), 8.27 (d, J=8.4 Hz, 1H), 8.05-7.96 (m, 4H), 7.50-7.43 (m, 6H), 7.42-7.37 (m, 1H), 7.16-7.13 (m, 1H), 7.03 (br s, 2H), 6.40-6.35 (m, 1H), 3.93-3.87 (m, 0.5H), 3.59 (s, 2H), 3.43-3.36 (m, 0.5H), 2.94-2.87 (m, 2H), 2.85 (s, 1.5H), 2.81 (s, 1.5H), 2.06-1.95 (m, 2H), 1.87-1.82 (m, 2H), 1.69-1.55 (m, 2H).

### Example 46 and 47: 3-((1-(4-(2-(2-Aminopyridin-3-yl)-5-phenyl-3H-imidazo[4,5-b]pyridin-3-yl)benzyl)piperidin-4-yl)(methyl)amino)-4-mercaptocyclobut-3-ene-1,2-dione and 3-((1-(4-(2-(2-Aminopyridin-3-yl)-5-phenyl-3H-imidazo[4,5-b]pyridin-3-yl)benzyl)piperidin-4-yl)(methyl)amino)-4-(methylthio)cyclobut-3-ene-1,2-dione

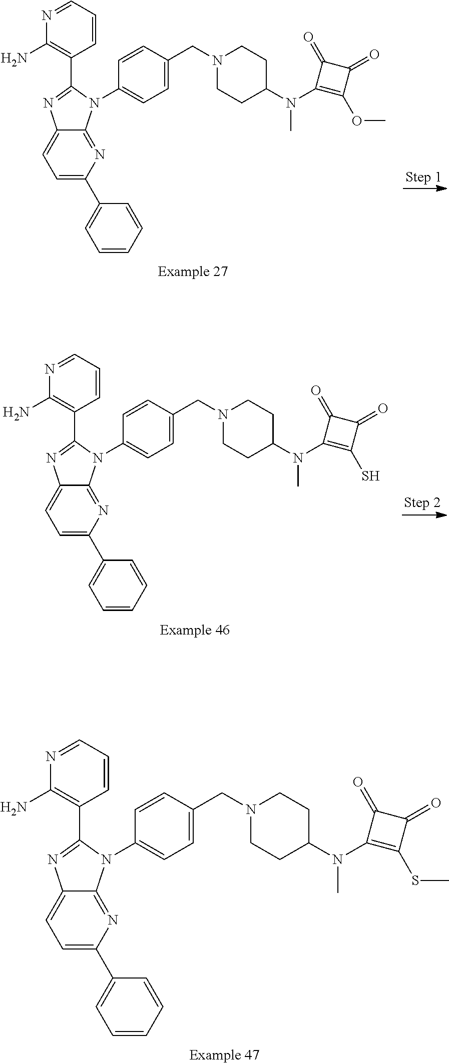 [SMILES: COc1c(N(C)C2CCN(Cc3ccc(-n4c(-c5cccnc5N)nc5ccc(-c6ccccc6)nc54)cc3)CC2)c(=O)c1=O ; CN(c1c(S)c(=O)c1=O)C1CCN(Cc2ccc(-n3c(-c4cccnc4N)nc4ccc(-c5ccccc5)nc43)cc2)CC1 ; CSc1c(N(C)C2CCN(Cc3ccc(-n4c(-c5cccnc5N)nc5ccc(-c6ccccc6)nc54)cc3)CC2)c(=O)c1=O]

### Step 1: 3-((1-(4-(2-(2-Aminopyridin-3-yl)-5-phenyl-3H-imidazo[4,5-b]pyridin-3-yl)benzyl)piperidin-4-yl)(methyl)amino)-4-mercaptocyclobut-3-ene-1,2-dione

To a solution of Example 27 (130 mg, 217 gmol) in EtOH (1 mL) was added NaSH (36.5 mg, 650 gmol). The mixture was stirred at 25° C. for 16 hr. The reaction was concentrated under reduced pressure. After purified by prep-HPLC (column: Waters xbridge 150×25 mm×10 μm; mobile phase: [water (NH₄HCO₃)-ACN]; B %: 23%-53%, 8 min), 3-((1-(4-(2-(2-aminopyridin-3-yl)-5-phenyl-3H-imidazo[4,5-b]pyridin-3-yl)benzyl)piperidin-4-yl)(methyl)amino)-4-mercaptocyclobut-3-ene-1,2-dione (Example 46, 35.3 mg, yield: 27%) was obtained as a yellow solid. MS: m/z=602.5 [M+H]⁺. ¹H NMR (400 MHz, Methanol-d₄) δ 8.22 (d, J=8.0 Hz, 1H), 8.04-8.02 (m, 2H), 7.96 (d, J=8.4 Hz, 1H), 7.68 (d, J=7.2 Hz, 2H), 7.60 (d, J=7.2 Hz, 2H), 7.46-7.35 (m, 5H), 6.58-6.50 (m, 1H), 4.66-4.56 (m, 2H), 4.28-4.21 (m, 1H), 3.75-3.54 (m, 2H), 3.48-3.36 (m, 3H), 3.10-2.88 (m, 2H), 2.21-2.01 (m, 4H).

### Step 2: 3-((1-(4-(2-(2-Aminopyridin-3-yl)-5-phenyl-3H-imidazo[4,5-b]pyridin-3-yl)benzyl)piperidin-4-yl)(methyl)amino)-4-(methylthio)cyclobut-3-ene-1,2-dione

To a solution of Example 46 (80 mg, 133 μmol) in EtOH (1 mL) was added CH₃I (56.6 mg, 399 μmol) at 0° C. The mixture was stirred at 25° C. for 2 hr. The reaction was concentrated under reduced pressure. After purified by prep-HPLC (column: Waters xbridge 150×25 mm×10 μm; mobile phase: [water (NH₄HCO₃)-ACN]; B %: 42%-72%, 8 min), 3-((1-(4-(2-(2-aminopyridin-3-yl)-5-phenyl-3H-imidazo[4,5-b]pyridin-3-yl)benzyl)piperidin-4-yl)(methyl)amino)-4-(methylthio)cyclobut-3-ene-1,2-dione (Example 47, 5.7 mg, yield: 6.6%) was obtained as a yellow solid. MS: m/z=616.4 [M+H]⁺. ¹H NMR (400 MHz, Methanol-d₄) δ 8.20 (d, J=8.8 Hz, 1H), 8.04 (d, J=7.2 Hz, 2H), 7.98-7.97 (m, 1H), 7.96-7.93 (m, 1H), 7.54 (d, J=8.0 Hz, 2H), 7.46-7.41 (m, 4H), 7.40-7.37 (m, 1H), 7.34-7.31 (m, 1H), 6.53-6.46 (m, 1H), 4.61-4.57 (m, 1H), 3.68-3.64 (m, 2H), 3.34 (s, 3H), 3.10-3.04 (m, 2H), 2.92-2.88 (m, 3H), 2.22-2.15 (m, 2H), 2.05-1.98 (m, 2H), 1.85-1.75 (m, 2H).

### Example 48: 3-((1-(4-(2-(2-Aminopyridin-3-yl)-5-phenyl-3H-imidazo[4,5-b]pyridin-3-yl)benzyl)piperidin-4-yl)(2-hydroxyethyl)amino)-4-methoxycyclobut-3-ene-1,2-dione

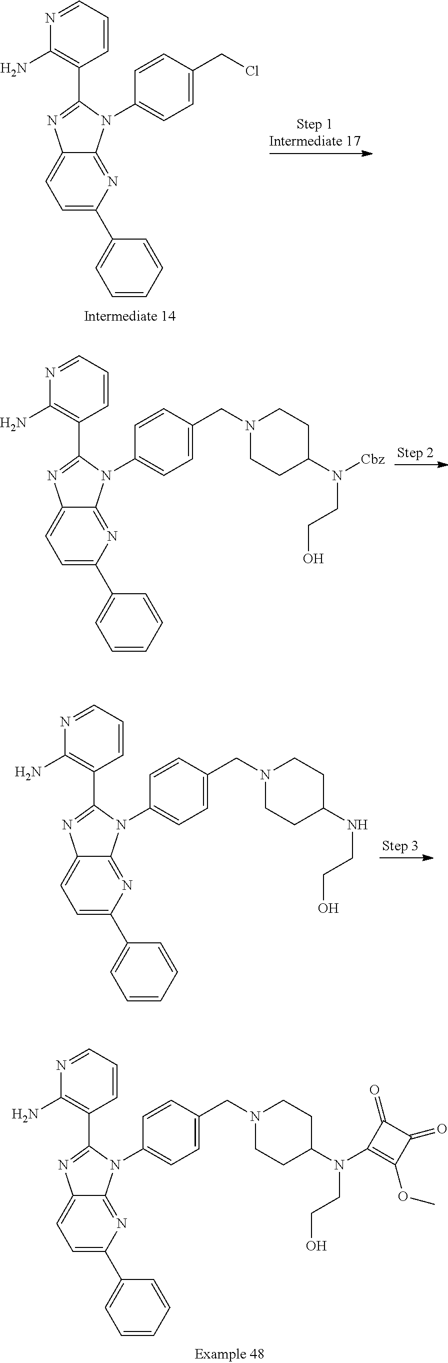 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCC(N(CCO)C(=O)OCc3ccccc3)CC2)cc1 ; Nc1ncccc1-c1nc2ccc(-c3ccccc3)nc2n1-c1ccc(CN2CCC(NCCO)CC2)cc1 ; COc1c(N(CCO)C2CCN(Cc3ccc(-n4c(-c5cccnc5N)nc5ccc(-c6ccccc6)nc54)cc3)CC2)c(=O)c1=O]

### Step 1: Benzyl (1-(4-(2-(2-aminopyridin-3-yl)-5-phenyl-3H-imidazo[4,5-b]pyridin-3-yl)benzyl)piperidin-4-yl)(2-hydroxyethyl)carbamate)

To a solution of Intermediate 14 (1.0 g, 2.43 mmol), Intermediate 17 (676 mg HCl salt, 2.15 mmol), and K₂CO₃ (1.0 g, 7.28 mmol) in DMF (10 mL) was added NaI (36 mg, 242 μmol). The mixture was stirred at 80° C. for 5 hr. The reaction mixture was diluted with H₂O (200 mL) and extracted with EtOAc (100 mL×2). The combined organic layers were washed with brine (100 mL), dried over Na₂SO₄, filtered, and concentrated under reduced pressure. After purified by silica gel flash chromatography (Eluent of 0~9% MeOH in CH₂Cl₂), benzyl (1-(4-(2-(2-aminopyridin-3-yl)-5-phenyl-3H-imidazo[4,5-b]pyridin-3-yl)benzyl)piperidin-4-yl)(2-hydroxyethyl)carbamate (500 mg, yield: 27%) was obtained as a yellow solid. MS: m/z=654.2 [M+H]⁺.

### Step 2: 2-((1-(4-(2-(2-Aminopyridin-3-yl)-5-phenyl-3H-imidazo[4,5-b]pyridin-3-yl)benzyl)piperidin-4-yl)amino)ethanol

A mixture of benzyl (1-(4-(2-(2-aminopyridin-3-yl)-5-phenyl-3H-imidazo[4,5-b]pyridin-3-yl)benzyl)piperidin-4-yl)(2-hydroxyethyl)carbamate (220 mg, 336 μmol) and Pd/C (30 mg, 10% purity) in MeOH (5 mL) was degassed and purged with H₂ three times. The mixture was stirred at 40° C. for 6 hr under H₂ atmosphere. The reaction mixture was filtered and concentrated under reduced pressure. After purified by prep-TLC (CH₂Cl₂ in MeOH=10%), 2-((1-(4-(2-(2-aminopyridin-3-yl)-5-phenyl-3H-imidazo[4,5-b]pyridin-3-yl)benzyl)piperidin-4-yl)amino)ethanol (11.4 mg, yield: 6%) was obtained as a light yellow solid. MS: m/z=534.3 [M+H]⁺. ¹H NMR (400 MHz, Methanol-d₄) δ 8.18 (d, J=8.4 Hz, 1H), 8.05-8.01 (m, 2H), 7.98 (dd, J=4.8, 1.6 Hz, 1H), 7.93 (d, J=8.4 Hz, 1H), 7.55-7.50 (m, 2H), 7.45-7.35 (m, 5H), 7.31 (dd, J=7.6, 1.6 Hz, 1H), 6.46 (dd, J=7.6, 4.8 Hz, 1H), 3.69-3.65 (m, 2H), 3.61 (s, 2H), 3.01-2.91 (m, 2H), 2.78 (t, J=5.6 Hz, 2H), 2.63-2.55 (m, 1H), 2.17-2.09 (m, 2H), 1.98-1.89 (m, 2H), 1.54-1.44 (m, 2H).

### Step 3: 3-((1-(4-(2-(2-Aminopyridin-3-yl)-5-phenyl-3H-imidazo[4,5-b]pyridin-3-yl)benzyl)piperidin-4-yl)(2-hydroxyethyl)amino)-4-methoxycyclobut-3-ene-1,2-dione

To a solution of 2-((1-(4-(2-(2-aminopyridin-3-yl)-5-phenyl-3H-imidazo[4,5-b]pyridin-3-yl)benzyl)piperidin-4-yl)amino)ethanol (85 mg, 140 gmol) in MeOH (1 mL) were added TEA (17 mg, 168 mol) and 3,4-dimethoxycyclobut-3-ene-1,2-dione (20 mg, 140 mol). The mixture was stirred at 25° C. for 1 hr. The reaction mixture was filtered and concentrated under reduced pressure. After purified by prep-TLC (CH₂Cl₂ in MeOH=10%), 3-((1-(4-(2-(2-aminopyridin-3-yl)-5-phenyl-3H-imidazo[4,5-b]pyridin-3-yl)benzyl)piperidin-4-yl)(2-hydroxyethyl)amino)-4-methoxycyclobut-3-ene-1,2-dione (Example 48, 30 mg, yield: 31%) was obtained as a yellow solid. MS: m/z=630.3 [M+H]⁺. ¹H NMR (400 MHz, Methanol-d₄) δ 8.19 (d, J=8.4 Hz, 1H), 8.05-8.01 (m, 2H), 7.98 (dd, J=4.8, 1.6 Hz, 1H), 7.94 (d, J=8.4 Hz, 1H), 7.57-7.52 (m, 2H), 7.47-7.31 (m, 6H), 6.53-6.45 (m, 1H), 4.38-4.36 (m, 3H), 3.85-3.78 (m, 1H), 3.76-3.61 (m, 5H), 3.60-3.55 (m, 1H), 3.06-3.01 (m, 2H), 2.22-2.12 (m, 2H), 2.05-1.95 (m, 2H), 1.90-1.78 (m, 2H).

### Example 49: 3-((1-(4-(2-(2-Aminopyridin-3-yl)-5-phenyl-3H-imidazo[4,5-b]pyridin-3-yl)benzyl)piperidin-4-yl)(methyl)amino)-4-(ethylthio)cyclobut-3-ene-1,2-dione

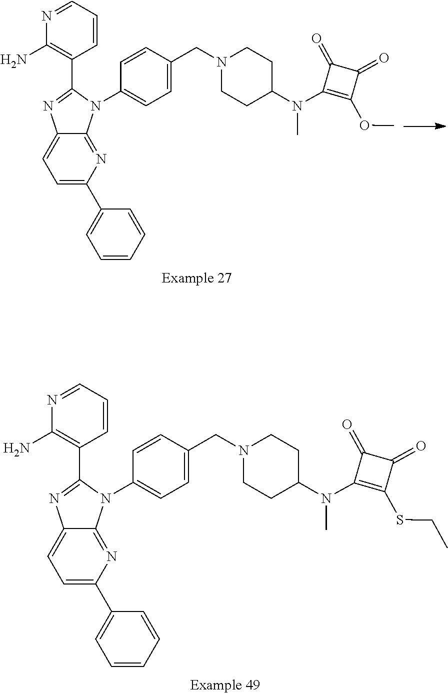 [SMILES: COc1c(N(C)C2CCN(Cc3ccc(-n4c(-c5cccnc5N)nc5ccc(-c6ccccc6)nc54)cc3)CC2)c(=O)c1=O ; CCSc1c(N(C)C2CCN(Cc3ccc(-n4c(-c5cccnc5N)nc5ccc(-c6ccccc6)nc54)cc3)CC2)c(=O)c1=O]

To a solution of Example 27 (200 mg, 334 μmol) in CH₂Cl₂ (5 mL) were added TEA (169 mg, 1.67 mmol) and EtSH (2.56 g, 41.2 mmol). The resulting mixture was stirred at 25° C. for 24 hr. The reaction mixture was quenched with H₂O (5 mL) at 0° C., diluted with CH₂Cl₂ (10 mL) and extracted with CH₂Cl₂ (15 mL×2). The combined organic layers were washed with brine (15 mL), dried over Na₂SO₄, filtered, and concentrated under reduced pressure. After purified by prep-HPLC (column: Waters xbridge 150×25 mm×10 μm; mobile phase: [water (NH₄HCO₃)-ACN]; B %: 44%-74%, 14 min), 3-((1-(4-(2-(2-aminopyridin-3-yl)-5-phenyl-3H-imidazo[4,5-b]pyridin-3-yl)benzyl)piperidin-4-yl)(methyl)amino)-4-(ethylthio)cyclobut-3-ene-1,2-dione (Example 49, 24.8 mg, yield: 11%) was obtained as a light-yellow lyophilized powder. MS: m/z=630.3 [M+H]⁺. ¹H NMR (400 MHz, Dimethylsulfoxide-d₆) δ 8.27 (d, J=8.4 Hz, 1H), 8.05-7.96 (m, 4H), 7.51-7.44 (m, 6H), 7.42-7.37 (m, 1H), 7.18-7.13 (m, 1H), 7.03 (br s, 2H), 6.42-6.36 (m, 1H), 4.44-4.28 (m, 0.5H), 3.70-3.63 (m, 0.5H), 3.60 (s, 2H), 3.45-3.39 (m, 2H), 3.25 (s, 1.5H), 3.13 (s, 1.5H), 2.95 (t, J=10.8 Hz, 2H), 2.09-2.01 (m, 2H), 1.96-1.84 (m, 2H), 1.76-1.67 (m, 2H), 1.35 (t, J=7.6 Hz, 3H).

### Example 50: (R)-3-((1-(4-(2-(2-Aminopyridin-3-yl)-5-phenyl-3H-imidazo[4,5-b]pyridin-3-yl)benzyl)pyrrolidin-3-yl)amino)-4-methoxycyclobut-3-ene-1,2-dione

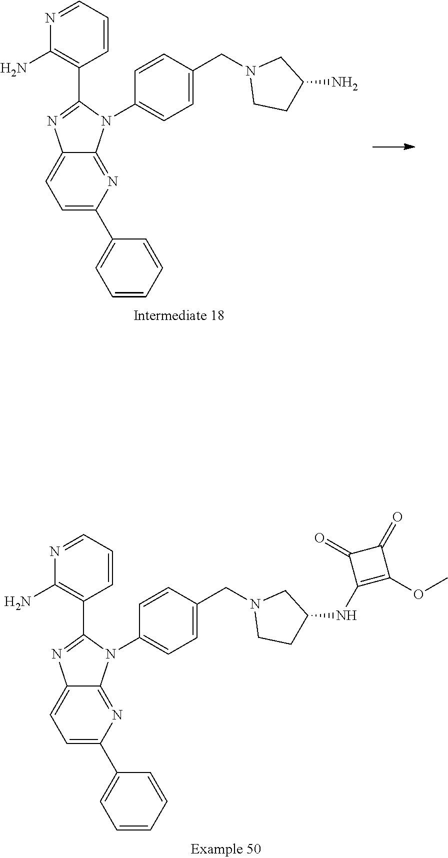 [SMILES: Nc1ncccc1-c1nc2ccc(-c3ccccc3)nc2n1-c1ccc(CN2CC[C@@H](N)C2)cc1 ; COc1c(N[C@@H]2CCN(Cc3ccc(-n4c(-c5cccnc5N)nc5ccc(-c6ccccc6)nc54)cc3)C2)c(=O)c1=O]

To a solution of Intermediate 18 (150 mg, 324 μmol) in MeOH (1 mL) were added 3,4-dimethoxycyclobut-3-ene-1,2-dione (55.4 mg, 389 μmol) and TEA (164 mg, 1.62 mmol). The mixture was stirred at 20° C. for 2 hr. The reaction mixture was filtered and concentrated under reduced pressure. The residue was purified by prep-HPLC (column: Phenomenex C18 150*25 mm*10 um; mobile phase: [water (NH₄HCO₃)-ACN]; B %: 35%-65%, 14 min) and then purified by prep-TLC (CH₂Cl₂:MeOH=10:1). (R)-3-((1-(4-(2-(2-aminopyridin-3-yl)-5-phenyl-3H-imidazo[4,5-b]pyridin-3-yl)benzyl)pyrrolidin-3-yl)amino)-4-methoxycyclobut-3-ene-1,2-dione (Example 50, 17.9 mg, yield: 32%) was obtained as a yellow solid. MS: m/z=572.2 [M+H]⁺. ¹H NMR (400 MHz, Methanol-d₄) δ 8.19 (d, J=8.8 Hz, 1H), 8.00 (d, J=7.2 Hz, 2H), 7.98 (dd, J=5.2, 2.0 Hz, 1H), 7.93 (d, J=8.4 Hz, 1H), 7.55 (d, J=8.4 Hz, 2H), 7.47-7.28 (m, 6H), 6.46 (d, J=7.6, 4.8 Hz, 1H), 4.36 (s, 3H), 3.77-3.73 (m, 1H), 3.33-3.32 (m, 2H), 2.92-2.80 (m, 2H), 2.70-2.56 (m, 2H), 2.42-2.30 (m, 1H), 1.90-1.78 (m, 1H).

### Example 51: 3-((1-(4-(2-(2-Aminopyridin-3-yl)-5-phenyl-3H-imidazo[4,5-b]pyridin-3-yl)benzyl)azetidin-3-yl)amino)-4-methoxycyclobut-3-ene-1,2-dione

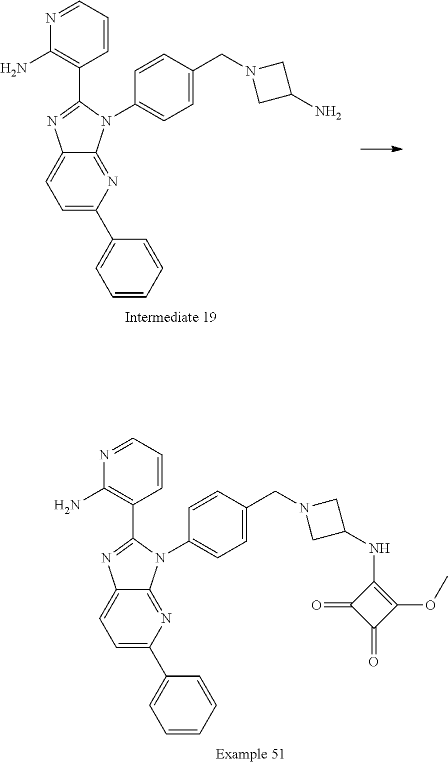 [SMILES: Nc1ncccc1-c1nc2ccc(-c3ccccc3)nc2n1-c1ccc(CN2CC(N)C2)cc1 ; COc1c(NC2CN(Cc3ccc(-n4c(-c5cccnc5N)nc5ccc(-c6ccccc6)nc54)cc3)C2)c(=O)c1=O]

To a solution of Intermediate 19 (150 mg, 335 μmol) in MeOH (1 mL) were added TEA (169 mg, 1.68 mmol) and 3,4-dimethoxycyclobut-3-ene-1,2-dione (57.1 mg, 402 μmol). The mixture was stirred at 25° C. for 2 hr. The reaction mixture was concentrated under reduced pressure. The residue was purified by silica gel flash chromatography (Eluent of 0~6% MeOH in CH₂Cl₂) and then purified again by prep-TLC (CH₂Cl₂:MeOH=10:1). 3-((1-(4-(2-(2-aminopyridin-3-yl)-5-phenyl-3H-imidazo[4,5-b]pyridin-3-yl)benzyl)azetidin-3-yl)amino)-4-methoxycyclobut-3-ene-1,2-dione (Example 51, 15.3 mg, yield: 8.0%, 1:1 mixture of tautomers) was obtained as a light-yellow solid. MS: m/z=558.3 [M+H]⁺. ¹H NMR (400 MHz, Methanol-d₄) δ 8.19 (d, J=8.4 Hz, 1H), 8.03 (d, J=7.2 Hz, 2H), 7.98 (dd, J=5.2, 1.6 Hz, 1H), 7.96-7.93 (m, 1H), 7.52-7.35 (m, 8H), 6.55-6.46 (m, 1H), 4.58 (s, 1.5H), 4.38 (s, 1.5H), 3.80-3.76 (m, 1H), 3.77-3.65 (m, 2H), 3.35 (s, 2H), 3.28-3.12 (m, 2H).

### Example 52: 3-((2-(4-(2-(2-Aminopyridin-3-yl)-5-phenyl-3H-imidazo[4,5-b]pyridin-3-yl)benzyl)-2-azaspiro[4.5]decan-8-yl)amino)-4-methoxycyclobut-3-ene-1,2-dione

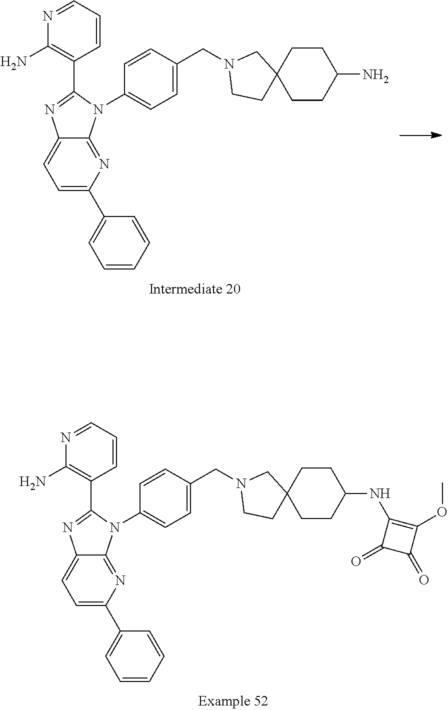 [SMILES: Nc1ncccc1-c1nc2ccc(-c3ccccc3)nc2n1-c1ccc(CN2CCC3(CCC(N)CC3)C2)cc1 ; COc1c(NC2CCC3(CC2)CCN(Cc2ccc(-n4c(-c5cccnc5N)nc5ccc(-c6ccccc6)nc54)cc2)C3)c(=O)c1=O]

To a solution of Intermediate 20 (100 mg, 189 μmol) in MeOH (2 mL) were added 3,4-dimethoxycyclobut-3-ene-1,2-dione (26.8 mg, 189 μmol) and TEA (42 mg, 415 mol). The reaction mixture was stirred at 25° C. for 2 hr. The mixture was concentrated under reduced pressure. The residue was purified by prep-TLC (CH₂Cl₂:MeOH=10:1) to give 3-((2-(4-(2-(2-Aminopyridin-3-yl)-5-phenyl-3H-imidazo[4,5-b]pyridin-3-yl)benzyl)-2-azaspiro[4.5]decan-8-yl)amino)-4-methoxycyclobut-3-ene-1,2-dione (Example 52, 20.5 mg, yield: 17%, 1:1 mixture of tautomers) as a light-yellow solid. MS: m/z=640.4 [M+H]⁺. ¹H NMR (400 MHz, Methanol-d₄) δ 8.19 (d, J=8.4 Hz, 1H), 8.03 (d, J=7.2 Hz, 2H), 7.98 (dd, J=4.8, 1.6 Hz, 1H), 7.94 (d, J=8.4 Hz, 1H), 7.57-7.52 (m, 2H), 7.46-7.43 (m, 2H), 7.43-7.31 (m, 4H), 6.50-6.44 (m, 1H), 4.38-4.29 (m, 3H), 3.92-3.85 (m, 0.5H), 3.78-3.68 (m, 2H), 3.53-3.48 (m, 0.5H), 2.76-2.68 (m, 2H), 2.58-2.46 (m, 2H), 1.86-1.84 (m, 2H), 1.80-1.68 (m, 4H), 1.54-1.42 (m, 4H).

### Example 53: 3-(7-(4-(2-(2-Aminopyridin-3-yl)-5-phenyl-3H-imidazo[4,5-b]pyridin-3-yl)benzyl)-2,7-diazaspiro[3.5]nonan-2-yl)-4-methoxycyclobut-3-ene-1,2-dione

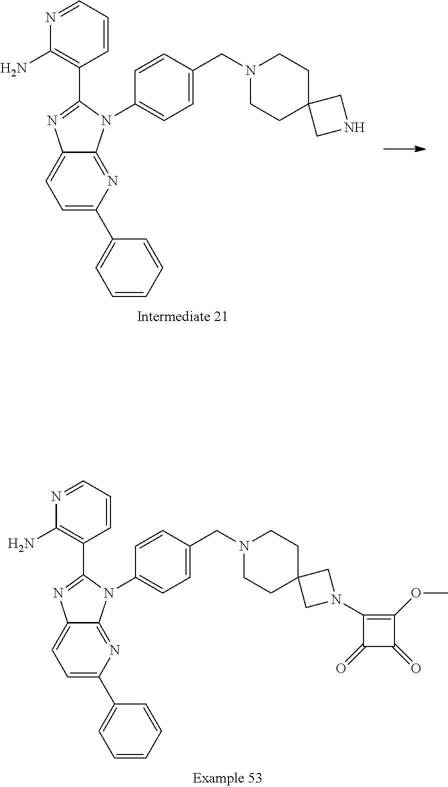 [SMILES: Nc1ncccc1-c1nc2ccc(-c3ccccc3)nc2n1-c1ccc(CN2CCC3(CC2)CNC3)cc1 ; COc1c(N2CC3(CCN(Cc4ccc(-n5c(-c6cccnc6N)nc6ccc(-c7ccccc7)nc65)cc4)CC3)C2)c(=O)c1=O]

To a solution of Intermediate 21 (100 mg, 199 μmol) and 3,4-dimethoxycyclobut-3-ene-1,2-dione (34 mg, 239 μmol) in MeOH (2 mL) was added TEA (101 mg, 997 μmol). The mixture was stirred at 25° C. for 1 hr. The reaction mixture was concentrated. The residue was purified by prep-TLC (CH₂Cl₂:MeOH=10:1) to give 3-(7-(4-(2-(2-aminopyridin-3-yl)-5-phenyl-3H-imidazo[4,5-b]pyridin-3-yl)benzyl)-2,7-diazaspiro[3.5]nonan-2-yl)-4-methoxycyclobut-3-ene-1,2-dione (Example 53, 12.7 mg, yield: 9.9%) as a light-yellow solid. MS: m/z=612.3 [M+H]⁺. ¹H NMR (400 MHz, Dimethylsulfoxide-d₆) δ 8.28 (d, J=8.4 Hz, 1H), 8.05-7.97 (m, 4H), 7.86-7.63 (m, 1H), 7.51-7.38 (m, 6H), 7.16 (dd, J=7.2, 2 Hz, 1H), 7.04-6.96 (m, 2H), 6.46-6.33 (m, 1H), 4.29 (s, 2H), 4.21 (s, 3H), 4.17-4.03 (m, 4H), 3.58-3.49 (m, 4H), 1.92-1.77 (m, 4H).

### Example 54: 3-(8-(4-(2-(2-Aminopyridin-3-yl)-5-phenyl-3H-imidazo[4,5-b]pyridin-3-yl)benzyl)-2,8-diazaspiro[4.5]decan-2-yl)-4-methoxycyclobut-3-ene-1,2-dione

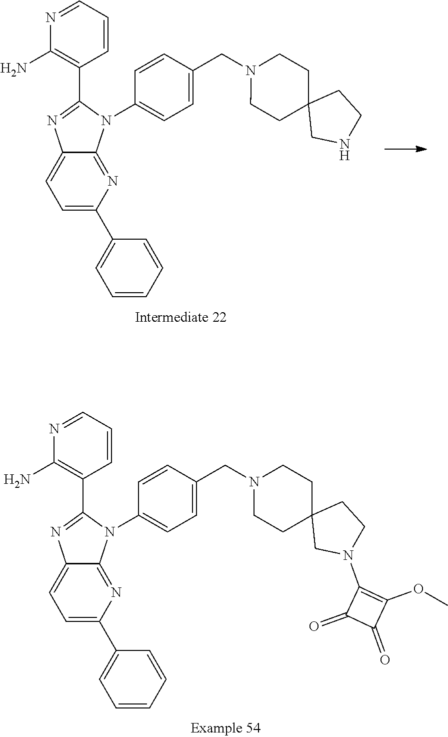 [SMILES: Nc1ncccc1-c1nc2ccc(-c3ccccc3)nc2n1-c1ccc(CN2CCC3(CCNC3)CC2)cc1 ; COc1c(N2CCC3(CCN(Cc4ccc(-n5c(-c6cccnc6N)nc6ccc(-c7ccccc7)nc65)cc4)CC3)C2)c(=O)c1=O]

To a solution of Intermediate 22 (100 mg, 194 μmol) and TEA (98 mg, 970 μmol) in MeOH (2 mL) was added 3,4-dimethoxycyclobut-3-ene-1,2-dione (33 mg, 233 μmol). The mixture was stirred at 25° C. for 2 hr. The residue was purified by prep-TLC (CH₂Cl₂:MeOH=10:1) to give 3-(8-(4-(2-(2-aminopyridin-3-yl)-5-phenyl-3H-imidazo[4,5-b]pyridin-3-yl)benzyl)-2,8-diazaspiro[4.5]decan-2-yl)-4-methoxycyclobut-3-ene-1,2-dione (Example 54, 10.0 mg, yield: 8%) as a yellow solid. MS: m/z=626.4 [M+H]⁺. ¹H NMR (400 MHz, Methanol-d₄) δ 8.19 (d, J=8.4 Hz, 1H), 8.06-8.01 (m, 2H), 7.98 (dd, J=5.2, 1.6 Hz, 1H), 7.94 (d, J=8.4 Hz, 1H), 7.54 (d, J=8.4 Hz, 2H), 7.46-7.30 (m, 6H), 6.51-6.43 (m, 1H), 4.34 (s, 3H), 3.88 (t, J=7.2 Hz, 1H), 3.72 (t, J=7.2 Hz, 1H), 3.69-3.67 (m, 1H), 3.65 (s, 2H), 3.51-3.47 (m, 1H), 2.66-2.56 (m, 2H), 2.48-2.38 (m, 2H), 1.91-1.84 (m, 2H), 1.75-1.67 (m, 4H).

### Example 55: 3-(6-(4-(2-(2-Aminopyridin-3-yl)-5-phenyl-3H-imidazo[4,5-b]pyridin-3-yl)benzyl)-2,6-diazaspiro[3.4]octan-2-yl)-4-methoxycyclobut-3-ene-1,2-dione

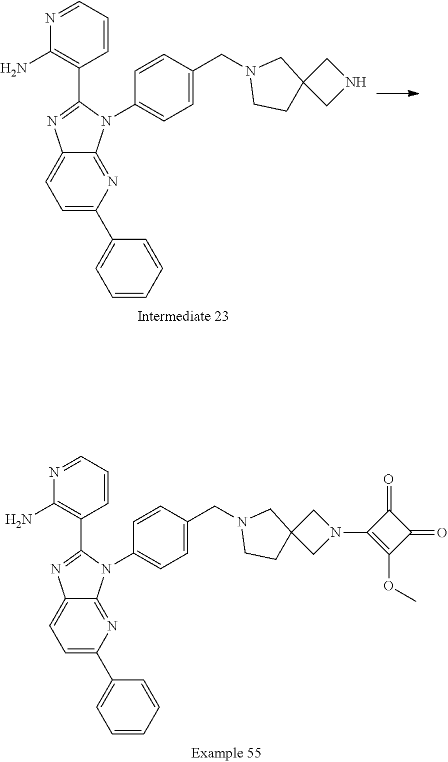 [SMILES: Nc1ncccc1-c1nc2ccc(-c3ccccc3)nc2n1-c1ccc(CN2CCC3(CNC3)C2)cc1 ; COc1c(N2CC3(CCN(Cc4ccc(-n5c(-c6cccnc6N)nc6ccc(-c7ccccc7)nc65)cc4)C3)C2)c(=O)c1=O]

To a solution of Intermediate 23 (80 mg, 164 μmol) and 3,4-dimethoxycyclobut-3-ene-1,2-dione (28 mg, 197 μmol) in MeOH (2 mL) was added TEA (49.8 mg, 492 μmol). The mixture was stirred at 25° C. for 2 hr. The mixture was concentrated under reduced pressure. After purified by prep-TLC (MeOH in CH₂Cl₂=10%), 3-(6-(4-(2-(2-aminopyridin-3-yl)-5-phenyl-3H-imidazo[4,5-b]pyridin-3-yl)benzyl)-2,6-diazaspiro[3.4]octan-2-yl)-4-methoxycyclobut-3-ene-1,2-dione (Example 55, 7.7 mg, yield: 8%) was obtained as a yellow solid. MS: m/z=598.3 [M+H]⁺. ¹H NMR (400 MHz, Methanol-d₄) δ 8.22-8.13 (m, 1H), 8.02 (d, J=8.0 Hz, 2H), 7.99-7.91 (m, 2H), 7.55-7.50 (m, 2H), 7.45-7.29 (m, 6H), 6.52-6.44 (m, 1H), 4.42-4.34 (m, 3H), 4.28 (s, 2H), 3.73 (s, 2H), 3.37-3.32 (m, 2H), 2.89 (s, 2H), 2.72-2.66 (m, 2H), 2.23-2.18 (m, 2H).

### Example 56: 3-(7-(4-(2-(2-Aminopyridin-3-yl)-5-phenyl-3H-imidazo[4,5-b]pyridin-3-yl)benzyl)-2,7-diazaspiro[4.4]nonan-2-yl)-4-methoxycyclobut-3-ene-1,2-dione

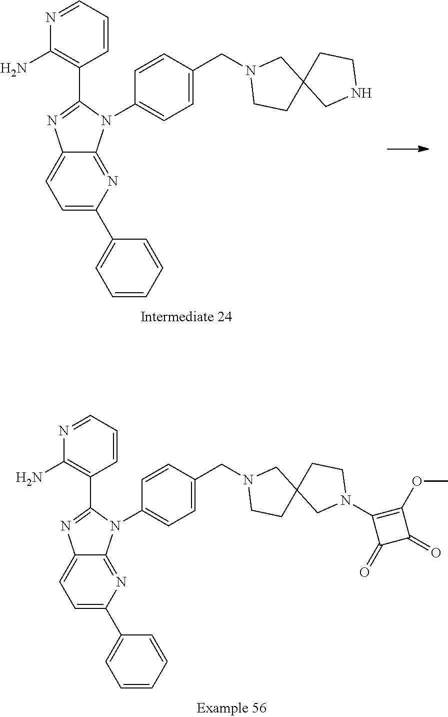 [SMILES: Nc1ncccc1-c1nc2ccc(-c3ccccc3)nc2n1-c1ccc(CN2CCC3(CCNC3)C2)cc1 ; COc1c(N2CCC3(CCN(Cc4ccc(-n5c(-c6cccnc6N)nc6ccc(-c7ccccc7)nc65)cc4)C3)C2)c(=O)c1=O]

To a solution of Intermediate 24 (200 mg, 399 μmol,) and 3,4-dimethoxycyclobut-3-ene-1,2-dione (68 mg, 478 μmol) in MeOH (1 mL) was added TEA (40 mg, 399 mol). The mixture was stirred at 25° C. for 2 hr. The mixture was concentrated under reduced pressure. The residue was purified by silica gel flash chromatography (Eluent of 0~80% EtOAc in petroleum ether), 3-(7-(4-(2-(2-aminopyridin-3-yl)-5-phenyl-3H-imidazo[4,5-b]pyridin-3-yl)benzyl)-2,7-diazaspiro[4.4]nonan-2-yl)-4-methoxycyclobut-3-ene-1,2-dione (Example 56, 26.2 mg, yield: 10%) was obtained as a yellow solid. MS: m/z=612.3 [M+H]⁺. 1H NMR (400 MHz, Dimethylsulfoxide-d₆) δ 8.26 (d, J=8.4 Hz, 1H), 8.08-7.94 (m, 4H), 7.51-7.37 (m, 7H), 7.15 (dd, J=7.6, 1.6 Hz, 1H), 7.01 (br s, 2H), 6.36 (dd, J=7.2, 4.8 Hz, 1H), 4.26 (s, 3H), 3.96-3.90 (m, 1H), 3.73-3.69 (m, 2H), 3.60-3.55 (m, 1H), 3.48-3.42 (m, 1H), 2.67-2.65 (m, 1H), 2.58-2.56 (m, 1H), 2.45-2.41 (m, 1H), 2.32-2.30 (m, 1H), 1.98-1.88 (m, 2H), 1.84-1.75 (m, 2H).

### Example 57: 3-(2-(4-(2-(2-Aminopyridin-3-yl)-5-phenyl-3H-imidazo[4,5-b]pyridin-3-yl)benzyl)-2,8-diazaspiro[4.5]decan-8-yl)-4-methoxycyclobut-3-ene-1,2-dione

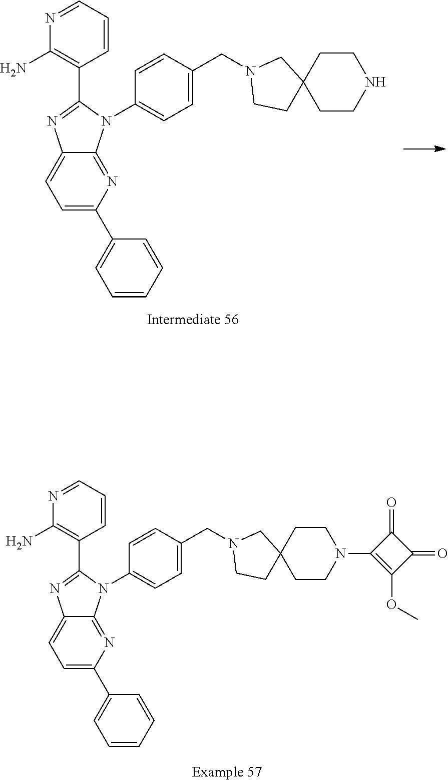 [SMILES: Nc1ncccc1-c1nc2ccc(-c3ccccc3)nc2n1-c1ccc(CN2CCC3(CCNCC3)C2)cc1 ; COc1c(N2CCC3(CCN(Cc4ccc(-n5c(-c6cccnc6N)nc6ccc(-c7ccccc7)nc65)cc4)C3)CC2)c(=O)c1=O]

To a solution of Intermediate 56 (100 mg, 186 μmol) and 3,4-dimethoxycyclobut-3-ene-1,2-dione (32 mg, 223 μmol) in MeOH (1 mL) was added TEA (19 mg, 186 μmol). The mixture was stirred at 25° C. for 2 hr. The mixture was filtered, washed with MeOH (3 mL), and concentrated under reduced pressure to give 3-(2-(4-(2-(2-aminopyridin-3-yl)-5-phenyl-3H-imidazo[4,5-b]pyridin-3-yl)benzyl)-2,8-diazaspiro[4.5]decan-8-yl)-4-methoxycyclobut-3-ene-1,2-dione (Example 57, 14.1 mg, yield: 12%) as a yellow solid. MS: m/z=626.4 [M+H]⁺. ¹H NMR (400 MHz, Dimethylsulfoxide-d₆) δ 8.27 (d, J=8.4 Hz, 1H), 8.08-7.96 (m, 4H), 7.51-7.41 (m, 6H), 7.41-7.34 (m, 1H), 7.17 (dd, J=7.6, 1.6 Hz, 1H), 7.01 (br s, 2H), 6.38 (dd, J=7.6, 4.8 Hz, 1H), 4.29 (s, 3H), 3.83-3.77 (m, 1H), 3.74-3.68 (m, 1H), 3.66 (s, 2H), 3.51-3.43 (m, 2H), 3.17 (s, 2H), 2.60-2.56 (m, 2H), 2.45-2.43 (m, 1H), 1.69-1.59 (m, 4H).

### Example 58: 3-(2-(4-(2-(2-Aminopyridin-3-yl)-5-phenyl-3H-imidazo[4,5-b]pyridin-3-yl)benzyl)-2,7-diazaspiro[4.5]decan-7-yl)-4-methoxycyclobut-3-ene-1,2-dione

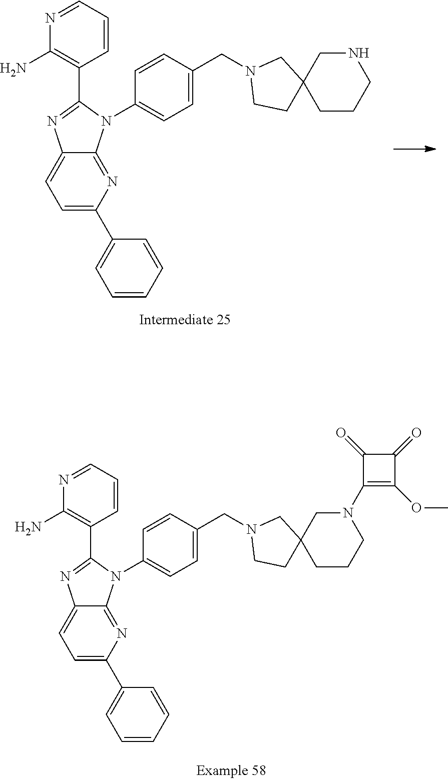 [SMILES: Nc1ncccc1-c1nc2ccc(-c3ccccc3)nc2n1-c1ccc(CN2CCC3(CCCNC3)C2)cc1 ; COc1c(N2CCCC3(CCN(Cc4ccc(-n5c(-c6cccnc6N)nc6ccc(-c7ccccc7)nc65)cc4)C3)C2)c(=O)c1=O]

To a solution of Intermediate 25 (160 mg, 319 μmol) and 3,4-dimethoxycyclobut-3-ene-1,2-dione (55 mg, 383 μmol) in MeOH (1 mL) was added TEA (33 mg, 319 μmol). The mixture was stirred at 25° C. for 2 hr. The mixture was filtered, washed with MeOH (3 mL), and concentrated under reduced pressure to give 3-(2-(4-(2-(2-Aminopyridin-3-yl)-5-phenyl-3H-imidazo[4,5-b]pyridin-3-yl)benzyl)-2,7-diazaspiro[4.5]decan-7-yl)-4-methoxycyclobut-3-ene-1,2-dione (Example 58, 17.5 mg, yield: 9%) as a yellow solid. MS: m/z=626.4 [M+H]⁺. ¹H NMR (400 MHz, Dimethylsulfoxide-d₆) δ 8.27 (d, J=8.4 Hz, 1H), 8.07-7.91 (m, 4H), 7.54-7.44 (m, 5H), 7.43-7.35 (m, 2H), 7.14 (t, J=5.6 Hz, 1H), 7.05 (br s, 1H), 7.01 (br s, 1H), 6.40-6.30 (m, 1H), 4.30 (s, 1.5H), 4.24 (s, 1.5H), 3.91-3.77 (m, 1H), 3.75-3.71 (m, 2H), 3.61-3.46 (m, 3H), 3.32-3.29 (m, 1H), 3.25-3.14 (m, 1H), 2.75-2.68 (m, 1H), 2.62-2.54 (m, 1H), 2.46-2.42 (m, 1H), 2.25-2.16 (m, 1H), 1.65-1.55 (m, 4H).

### Example 59: 3-(2-(4-(2-(2-Aminopyridin-3-yl)-5-phenyl-3H-imidazo[4,5-b]pyridin-3-yl)benzyl)-2,7-diazaspiro[3.5]nonan-7-yl)-4-ethoxycyclobut-3-ene-1,2-dione

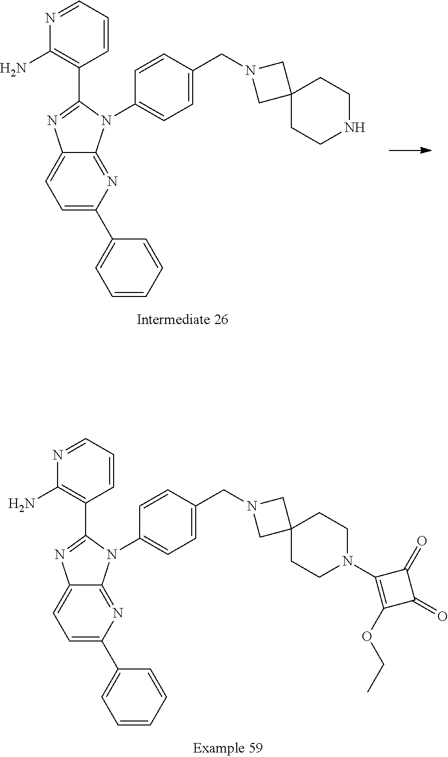 [SMILES: Nc1ncccc1-c1nc2ccc(-c3ccccc3)nc2n1-c1ccc(CN2CC3(CCNCC3)C2)cc1 ; CCOc1c(N2CCC3(CC2)CN(Cc2ccc(-n4c(-c5cccnc5N)nc5ccc(-c6ccccc6)nc54)cc2)C3)c(=O)c1=O]

To a solution of Intermediate 26 (100 mg, 199 mol) and 3,4-diethoxycyclobut-3-ene-1,2-dione (40.7 mg, 239 μmol) in MeOH (2 mL) was added TEA (101 mg, 997 μmol). The mixture was stirred at 25° C. for 14 hr. The mixture was filtrated. The solid was collected, washed with MeOH (10 mL×2), and dried under reduced pressure. After purified by silica gel flash chromatography (MeOH in CH₂Cl₂=1% to 5%), 3-(2-(4-(2-(2-aminopyridin-3-yl)-5-phenyl-3H-imidazo[4,5-b]pyridin-3-yl)benzyl)-2,7-diazaspiro[3.5]nonan-7-yl)-4-ethoxycyclobut-3-ene-1,2-dione (Example 59, 15.6 mg, yield: 14%) was obtained as a yellow solid. MS: m/z=626.3 [M+H]⁺. ¹H NMR (400 MHz, Methanol-d₄) δ 8.16 (d, J=8.4 Hz, 1H), 8.04-8.00 (m, 2H), 7.97 (dd, J=5.2, 1.6 Hz, 1H), 7.92 (d, J=8.4 Hz, 1H), 7.50-7.46 (m, 2H), 7.45-7.33 (m, 5H), 7.29 (dd, J=7.6, 2.0 Hz, 1H), 6.45 (dd, J=8.0, 5.2 Hz, 1H), 4.71 (q, J=7.2 Hz, 2H), 3.83-3.78 (m, 2H), 3.77 (s, 2H), 3.59-3.54 (m, 2H), 3.23-3.18 (m, 4H), 1.93-1.84 (m, 4H), 1.43 (t, J=7.2 Hz, 3H).

### Example 60: 3-(2-(4-(2-(2-Aminopyridin-3-yl)-5-phenyl-3H-imidazo[4,5-b]pyridin-3-yl)benzyl)-2,7-diazaspiro[3.5]nonan-7-yl)-4-methoxycyclobut-3-ene-1,2-dione

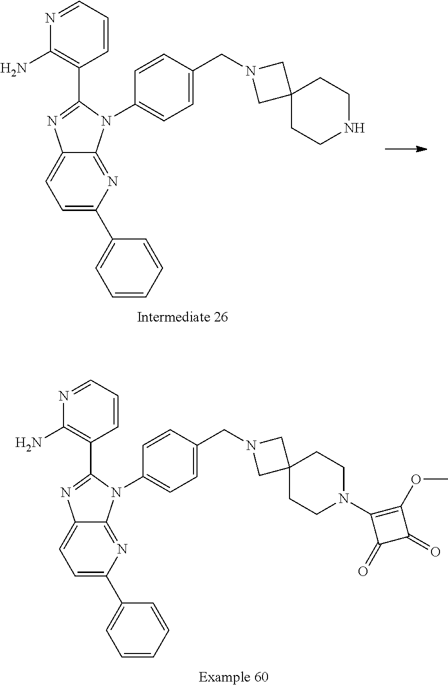 [SMILES: Nc1ncccc1-c1nc2ccc(-c3ccccc3)nc2n1-c1ccc(CN2CC3(CCNCC3)C2)cc1 ; COc1c(N2CCC3(CC2)CN(Cc2ccc(-n4c(-c5cccnc5N)nc5ccc(-c6ccccc6)nc54)cc2)C3)c(=O)c1=O]

To a solution of Intermediate 26 (80 mg, 159 μmol) in MeOH (1 mL) were added TEA (81 mg, 797 μmol) and 3,4-dimethoxycyclobut-3-ene-1,2-dione (27 mg, 191 mol). The reaction mixture was stirred at 25° C. for 16 hr. The mixture was filtered, washed with MeOH (2 mL×2) and dried under reduced pressure. 3-(2-(4-(2-(2-Aminopyridin-3-yl)-5-phenyl-3H-imidazo[4,5-b]pyridin-3-yl)benzyl)-2,7-diazaspiro[3.5]nonan-7-yl)-4-methoxycyclobut-3-ene-1,2-dione (Example 60, 24 mg, yield: 24%) was obtained as a yellow solid. MS: m/z=612.4 [M+H]⁺. ¹H NMR (400 MHz, Dimethylsulfoxide-d₆) δ 8.26 (d, J=8.0 Hz, 1H), 8.03-7.95 (m, 4H), 7.49-7.39 (m, 7H), 7.16 (dd, J=7.6, 1.6 Hz, 1H), 6.96 (br s, 2H), 6.40 (dd, J=7.6, 4.8 Hz, 1H), 4.29 (s, 3H), 3.74-3.69 (m, 2H), 3.66 (s, 2H), 3.53-3.50 (m, 2H), 3.05 (s, 4H), 1.84-1.77 (m, 4H).

### Example 61: 3-(2-(4-(2-(2-Aminopyridin-3-yl)-5-phenyl-3H-imidazo[4,5-b]pyridin-3-yl)benzyl)-2,7-diazaspiro[3.5]nonan-7-yl)-4-(ethylthio)cyclobut-3-ene-1,2-dione

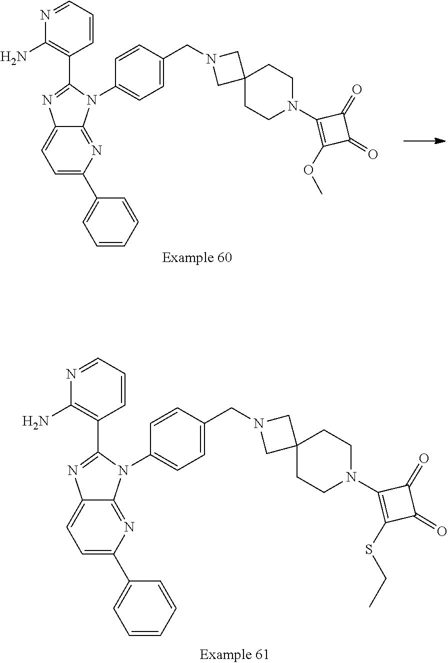 [SMILES: COc1c(N2CCC3(CC2)CN(Cc2ccc(-n4c(-c5cccnc5N)nc5ccc(-c6ccccc6)nc54)cc2)C3)c(=O)c1=O ; CCSc1c(N2CCC3(CC2)CN(Cc2ccc(-n4c(-c5cccnc5N)nc5ccc(-c6ccccc6)nc54)cc2)C3)c(=O)c1=O]

To a solution of Example 60 (130 mg, 213 mol) in CH₂Cl₂ (2 mL) were added TEA (108 mg, 1.06 mmol) and EtSH (1.95 g, 31.4 mmol). The mixture was stirred at 25° C. for 16 hr. The reaction mixture was quenched with H₂O (10 mL) at 25° C., extracted with CH₂Cl₂ (15 mL), washed with H₂O (20 mL×3) and brine (20 mL), dried over Na₂SO₄, filtered, and concentrated under reduced pressure. After purified by prep-HPLC (column: Phenomenex C18 150×25 mm×10 μm; mobile phase: [water (NH₄HCO₃)-ACN]; B %: 47%-77%, over 16 min) and prep-TLC (MeOH in CH₂Cl₂=10%), 3-(2-(4-(2-(2-aminopyridin-3-yl)-5-phenyl-3H-imidazo[4,5-b]pyridin-3-yl)benzyl)-2,7-diazaspiro[3.5]nonan-7-yl)-4-(ethylthio)cyclobut-3-ene-1,2-dione (Example 61, 2.2 mg, yield: 1.6%) was obtained as a light-yellow solid. MS: m/z=642.4 [M+H]⁺. ¹H NM/R (400 MHz, Methanol-d₄) δ 8.19 (d, J=8.4 Hz, 1H), 8.06-8.01 (m, 2H), 7.98 (dd, J=5.2, 2.0 Hz, 1H), 7.95 (d, J=8.4 Hz, 1H), 7.52-7.37 (m, 7H), 7.33 (dd, J=8.0, 2.0 Hz, 1H), 6.48 (dd, J=7.6, 4.8 Hz, 1H), 3.95-3.89 (m, 2H), 3.81 (s, 2H), 3.67-3.63 (m, 2H), 3.47 (q, J=7.2 Hz, 2H), 3.27-3.25 (m, 4H), 1.98-1.91 (m, 4H), 1.43 (t, J=7.2 Hz, 3H).

### Example 62: (R)-3-(7-(4-(2-(2-Aminopyridin-3-yl)-5-phenyl-3H-imidazo[4,5-b]pyridin-3-yl)benzyl)-2,7-diazaspiro[4.4]nonan-2-yl)-4-methoxycyclobut-3-ene-1,2-dione

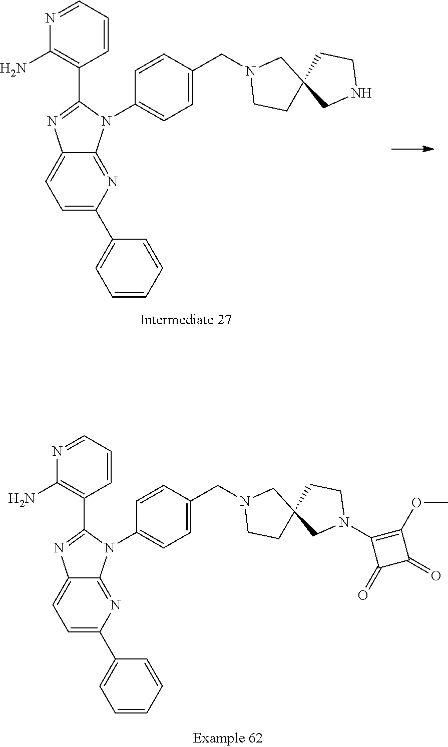 [SMILES: Nc1ncccc1-c1nc2ccc(-c3ccccc3)nc2n1-c1ccc(CN2CC[C@@]3(CCNC3)C2)cc1 ; COc1c(N2CC[C@@]3(CCN(Cc4ccc(-n5c(-c6cccnc6N)nc6ccc(-c7ccccc7)nc65)cc4)C3)C2)c(=O)c1=O]

To a solution of Intermediate 27 (70 mg, 140 μmol) in MeOH (1 mL) were added TEA (71 mg, 698 mol) and 3,4-dimethoxycyclobut-3-ene-1,2-dione (24 mg, 167 mol). The reaction mixture was stirred at 25° C. for 16 hr and then was filtered. The collected solid was washed with MeOH (2 mL×2) and dried under reduced pressure to give (R)-3-(7-(4-(2-(2-aminopyridin-3-yl)-5-phenyl-3H-imidazo[4,5-b]pyridin-3-yl)benzyl)-2,7-diazaspiro[4.4]nonan-2-yl)-4-methoxycyclobut-3-ene-1,2-dione (Example 62, 18.2 mg, yield: 21%) as a yellow solid. MS: m/z=612.3 [M+H]⁺. ¹H NMR (400 MHz, Dimethylsulfoxide-d₆) δ 8.27 (d, J=8.0 Hz, 1H), 8.04-7.97 (m, 4H), 7.50-7.43 (m, 6H), 7.41-7.37 (m, 1H), 7.15 (dd, J=7.6, 1.6 Hz, 1H), 7.02 (br s, 2H), 6.36 (dd, J=7.6, 4.8 Hz, 1H), 4.26 (s, 3H), 3.71-3.67 (m, 2H), 3.61-3.51 (m, 2H), 3.48-3.40 (m, 2H), 2.69-2.65 (m, 1H), 2.57-2.54 (m, 2H), 2.45-2.40 (m, 1H), 1.99-1.89 (m, 2H), 1.85-1.76 (m, 2H).

### Example 63: 3-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)-4-methoxycyclobut-3-ene-1,2-dione

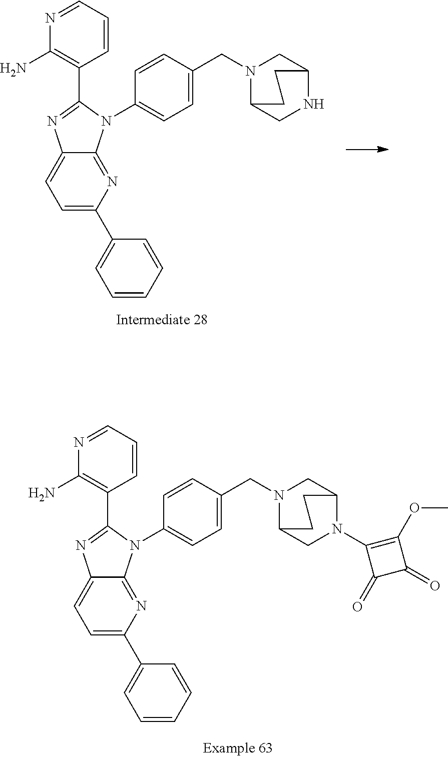 [SMILES: Nc1ncccc1-c1nc2ccc(-c3ccccc3)nc2n1-c1ccc(CN2CC3CCC2CN3)cc1 ; COc1c(N2CC3CCC2CN3Cc2ccc(-n3c(-c4cccnc4N)nc4ccc(-c5ccccc5)nc43)cc2)c(=O)c1=O]

To a solution of Intermediate 28 (70 mg, 144 μmol) in MeOH (1 mL) were added TEA (73 mg, 718 mol) and 3,4-dimethoxycyclobut-3-ene-1,2-dione (25 mg, 172 mol). The reaction mixture stirred at 25° C. for 16 hr. The mixture was filtered. The collected solid residue was washed with MeOH (2 mL×2) and dried under reduced pressure. 3-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)-4-methoxycyclobut-3-ene-1,2-dione (Example 63, 29.1 mg, yield: 33%) was obtained as a white solid. MS: m/z=598.3 [M+H]⁺. ¹H NMR (400 MHz, Dimethylsulfoxide-d₆) δ 8.27 (d, J=8.4 Hz, 1H), 8.04-7.97 (m, 4H), 7.53-7.50 (m, 2H), 7.49-7.43 (m, 4H), 7.42-7.37 (m, 1H), 7.17-7.14 (m, 1H), 7.01 (br s, 2H), 6.42-6.37 (m, 1H), 4.44-4.34 (m, 1H), 4.30-4.26 (m, 3H), 4.17-3.99 (m, 1H), 3.89-3.83 (m, 2H), 3.67-3.58 (m, 1H), 3.01-2.96 (m, 2H), 2.93-2.88 (m, 1H), 2.11-2.03 (m, 1H), 1.95-1.84 (m, 2H), 1.76-1.68 (m, 1H).

### Example 64: 3-(2-(4-(2-(2-Aminopyridin-3-yl)-5-phenyl-3H-imidazo[4,5-b]pyridin-3-yl)benzyl)-2,7-diazaspiro[3.5]nonan-7-yl)-4-(methylthio)cyclobut-3-ene-1,2-dione

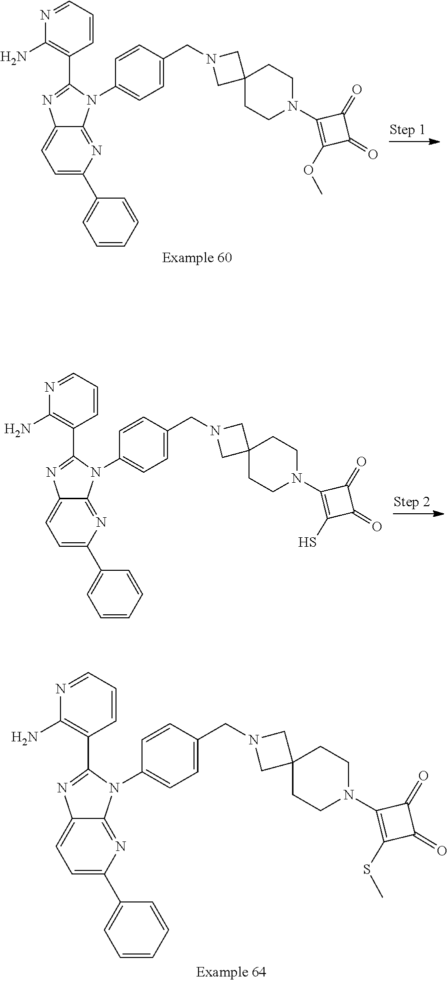 [SMILES: COc1c(N2CCC3(CC2)CN(Cc2ccc(-n4c(-c5cccnc5N)nc5ccc(-c6ccccc6)nc54)cc2)C3)c(=O)c1=O ; Nc1ncccc1-c1nc2ccc(-c3ccccc3)nc2n1-c1ccc(CN2CC3(CCN(c4c(S)c(=O)c4=O)CC3)C2)cc1 ; CSc1c(N2CCC3(CC2)CN(Cc2ccc(-n4c(-c5cccnc5N)nc5ccc(-c6ccccc6)nc54)cc2)C3)c(=O)c1=O]

### Step 1: 3-(2-(4-(2-(2-Aminopyridin-3-yl)-5-phenyl-3H-imidazo[4,5-b]pyridin-3-yl)benzyl)-2,7-diazaspiro[3.5]nonan-7-yl)-4-mercaptocyclobut-3-ene-1,2-dione

To a solution of Example 60 (120 mg, 196 gmol) in EtOH (1 mL) was added sulfanylsodium (55 mg, 980 μmol). The mixture was stirred at 25° C. for 16 hr. The reaction mixture was quenched with H₂O (10 mL) at 25° C., diluted with CH₂Cl₂ (15 mL), washed with H₂O (20 mL×3) and brine (20 mL), dried over Na₂SO₄, filtered and concentrated under reduced pressure. 3-(2-(4-(2-(2-aminopyridin-3-yl)-5-phenyl-3H-imidazo[4,5-b]pyridin-3-yl)benzyl)-2,7-diazaspiro[3.5]nonan-7-yl)-4-mercaptocyclobut-3-ene-1,2-dione (120 mg, yield: 55%) was obtained as a yellow solid. MS: m/z=614.2 [M+H]⁺.

### Step 2: 3-(2-(4-(2-(2-Aminopyridin-3-yl)-5-phenyl-3H-imidazo[4,5-b]pyridin-3-yl)benzyl)-2,7-diazaspiro[3.5]nonan-7-yl)-4-(methylthio)cyclobut-3-ene-1,2-dione

To a solution of 3-(2-(4-(2-(2-aminopyridin-3-yl)-5-phenyl-3H-imidazo[4,5-b]pyridin-3-yl)benzyl)-2,7-diazaspiro[3.5]nonan-7-yl)-4-mercaptocyclobut-3-ene-1,2-dione (120 mg, 196 gmol) in EtOH (4 mL) was added CH₃I (83.5 mg, 589 gmol). The mixture was stirred at 25° C. for 2 hr. The reaction was concentrated under reduced pressure. After purified by prep-HPLC (column: Waters xbridge 150×25 mm×10 μm; mobile phase: [water (NH₄HCO₃)-ACN]; B %: 37%-67%, over 14 min), 3-(2-(4-(2-(2-aminopyridin-3-yl)-5-phenyl-3H-imidazo[4,5-b]pyridin-3-yl)benzyl)-2,7-diazaspiro[3.5]nonan-7-yl)-4-(methylthio)cyclobut-3-ene-1,2-dione (Example 64, 6.6 mg, yield: 4.8%) was obtained as a yellow solid. MS: m/z=628.4 [M+H]⁺. ¹H NMR (400 MHz, Methanol-d₄) δ 8.19 (d, J=8.4 Hz, 1H), 8.05-8.01 (m, 2H), 7.98 (dd, J=4.8, 1.6 Hz, 1H), 7.95 (d, J=8.4 Hz, 1H), 7.54-7.46 (m, 4H), 7.45-7.36 (m, 3H), 7.34 (dd, J=8.0, 1.6 Hz, 1H), 6.48 (dd, J=7.6, 5.2 Hz, 1H), 3.94-3.89 (m, 2H), 3.83 (s, 2H), 3.65-3.60 (m, 2H), 3.29-3.28 (m, 4H), 2.90 (s, 3H), 1.99-1.91 (m, 4H).

### Example 65: 3-(2-(4-(2-(2-Aminopyridin-3-yl)-5-phenyl-3H-imidazo[4,5-b]pyridin-3-yl)benzyl)-2,6-diazaspiro[3.5]nonan-6-yl)-4-methoxycyclobut-3-ene-1,2-dione

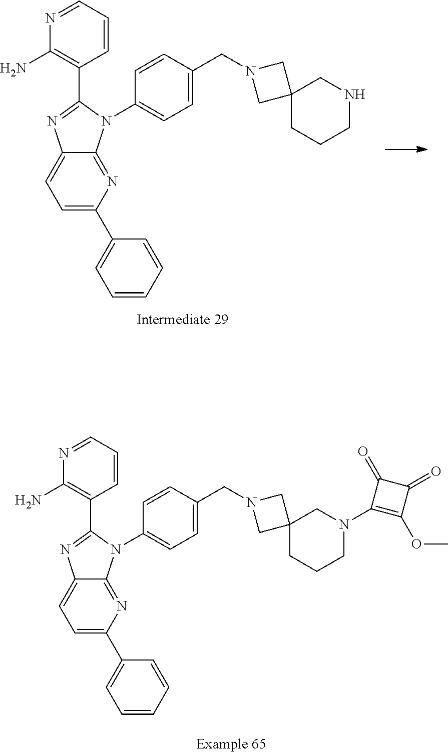 [SMILES: Nc1ncccc1-c1nc2ccc(-c3ccccc3)nc2n1-c1ccc(CN2CC3(CCCNC3)C2)cc1 ; COc1c(N2CCCC3(CN(Cc4ccc(-n5c(-c6cccnc6N)nc6ccc(-c7ccccc7)nc65)cc4)C3)C2)c(=O)c1=O]

To a solution of 3,4-dimethoxycyclobut-3-ene-1,2-dione (27.2 mg, 191 μmol) and Intermediate 29 (80.0 mg, 159 μmol) in MeOH (2 mL) was added TEA (80.7 mg, 797 μmol). The mixture was stirred at 25° C. for 16 hr. The reaction was filtered and concentrated under reduced pressure. The residue was purified by silica gel flash chromatography (Eluent of 0-10% MeOH in CH₂Cl₂) to give 3-(2-(4-(2-(2-aminopyridin-3-yl)-5-phenyl-3H-imidazo[4,5-b]pyridin-3-yl)benzyl)-2,6-diazaspiro[3.5]nonan-6-yl)-4-methoxycyclobut-3-ene-1,2-dione (Example 65, 36 mg, yield: 37%) as a yellow solid. MS: m/z=612.3 [M+H]⁺. ¹H NMR (400 MHz, Dimethylsulfoxide-d₆) δ 8.31-8.28 (m, 1H), 8.02-7.98 (m, 4H), 7.51-7.30 (m, 7H), 7.15 (dd, J=7.6, 1.6 Hz, 1H), 6.99 (br s, 2H), 6.41-6.36 (m, 1H), 4.35-4.30 (m, 1.5H), 4.31-4.20 (m, 1.5H), 3.92-3.88 (m, 1H), 3.75-3.65 (m, 4H), 3.43-3.40 (m, 1H), 3.10-3.00 (m, 2H), 2.95-2.85 (m, 2H), 1.80-1.70 (m, 2H), 1.60-1.50 (m, 2H).

### Example 66: (S)-3-(7-(4-(2-(2-aminopyridin-3-yl)-5-phenyl-3H-imidazo[4,5-b]pyridin-3-yl)benzyl)-2,7-diazaspiro[4.4]nonan-2-yl)-4-methoxycyclobut-3-ene-1,2-dione

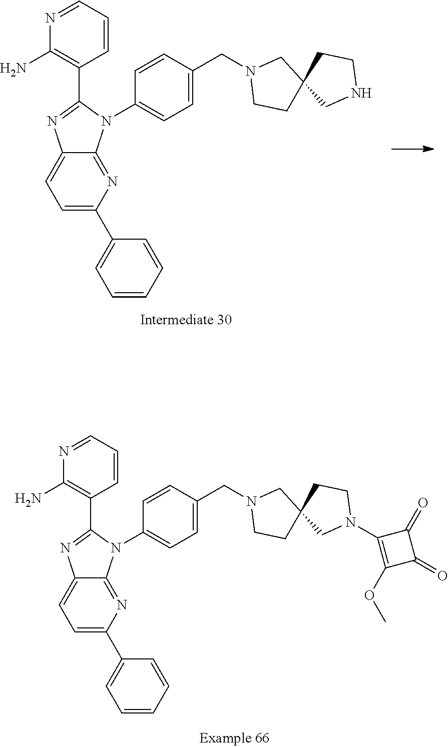 [SMILES: Nc1ncccc1-c1nc2ccc(-c3ccccc3)nc2n1-c1ccc(CN2CC[C@]3(CCNC3)C2)cc1 ; COc1c(N2CC[C@]3(CCN(Cc4ccc(-n5c(-c6cccnc6N)nc6ccc(-c7ccccc7)nc65)cc4)C3)C2)c(=O)c1=O]

To a solution of Intermediate 30 (50 mg, 99.6 μmol) and 3,4-dimethoxycyclobut-3-ene-1,2-dione (17.0 mg, 119 μmol) in MeOH (1 mL) was added TEA (50.4 mg, 498 μmol). The mixture was stirred at 25° C. for 16 hr. The reaction was filtered and concentrated under reduced pressure. The residue was purified by silica gel flash chromatography (Eluent of 0-10% MeOH in CH₂Cl₂) to give (S)-3-(7-(4-(2-(2-aminopyridin-3-yl)-5-phenyl-3H-imidazo[4,5-b]pyridin-3-yl)benzyl)-2,7-diazaspiro[4.4]nonan-2-yl)-4-methoxycyclobut-3-ene-1,2-dione (Example 66, 29.3 mg, yield: 48%) as a yellow solid. MS: m/z=612.2 [M+H]⁺. ¹H NMR (400 MHz, Methanol-d₄) δ 8.19 (d, J=8.4 Hz, 1H), 8.05-8.02 (m, 2H), 7.97 (dd, J=4.8, 1.6 Hz, 1H), 7.94 (d, J=8.4 Hz, 1H), 7.54 (d, J=8.4 Hz, 1H), 7.46-7.30 (m, 6H), 6.45 (dd, J=7.2, 4.8 Hz, 1H), 4.32 (s, 3H), 3.89-3.61 (m, 6H), 2.85-2.65 (m, 3H), 2.55 (d, J=9.6 Hz, 1H), 2.01-1.89 (m, 4H).

### Example 67: (R)-3-(4-(4-(2-(2-Aminopyridin-3-yl)-5-phenyl-3H-imidazo[4,5-b]pyridin-3-yl)benzyl)-2-methylpiperazin-1-yl)-4-methoxycyclobut-3-ene-1,2-dione

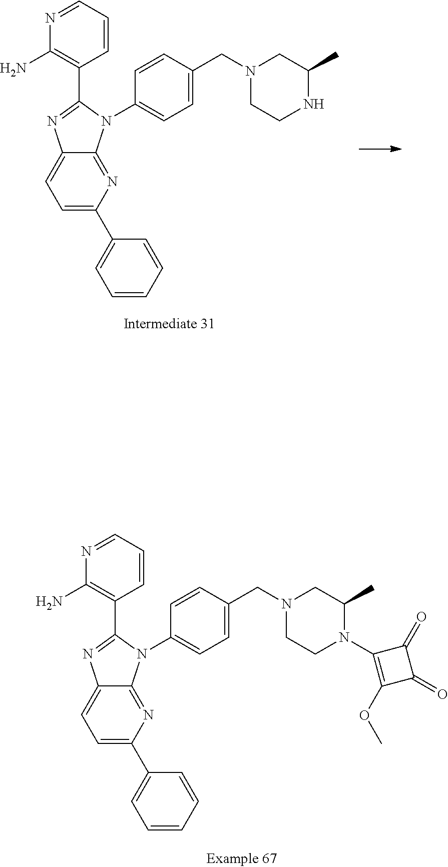 [SMILES: C[C@@H]1CN(Cc2ccc(-n3c(-c4cccnc4N)nc4ccc(-c5ccccc5)nc43)cc2)CCN1 ; COc1c(N2CCN(Cc3ccc(-n4c(-c5cccnc5N)nc5ccc(-c6ccccc6)nc54)cc3)C[C@H]2C)c(=O)c1=O]

To a solution of Intermediate 31 (55 mg, 116 μmol) in MeOH (1 mL) were added TEA (58.5 mg, 578 mol) and 3,4-dimethoxycyclobut-3-ene-1,2-dione (19.7 mg, 139 μmol). The reaction mixture stirred at 25° C. for 16 hr. Then the mixture was filtered. The collected solid residue was washed with MeOH (2 mL×2) and dried in vacuum to give (R)-3-(4-(4-(2-(2-aminopyridin-3-yl)-5-phenyl-3H-imidazo[4,5-b]pyridin-3-yl)benzyl)-2-methylpiperazin-1-yl)-4-methoxycyclobut-3-ene-1,2-dione (Example 67, 25.5 mg, yield: 37%) as a brown solid. MS: m/z=586.3 [M+H]⁺. ¹H NMR (400 MHz, Dimethylsulfoxide-d₆) δ 8.27 (d, J=8.4 Hz, 1H), 8.04-7.98 (m, 4H), 7.51-7.44 (m, 6H), 7.41-7.38 (m, 1H), 7.16 (dd, J=7.6, 1.6 Hz, 1H), 7.02 (br s, 2H), 6.39 (dd, J=7.6, 4.8 Hz, 1H), 4.67-4.58 (m, 0.5H), 4.30 (s, 3H), 4.29-4.17 (m, 1H), 4.08-3.98 (m, 0.5H), 3.67 (d, J=13.6 Hz, 1H), 3.55 (d, J=13.6 Hz, 1H), 3.47-3.42 (m, 1H), 2.93-2.90 (m, 1H), 2.69-2.66 (m, 1H), 2.35-2.30 (m, 1H), 2.26-2.18 (m, 1H), 1.36 (d, J=6.8 Hz, 3H).

### Example 68: 3-((2S,6R)-4-(4-(2-(2-aminopyridin-3-yl)-5-phenyl-3H-imidazo[4,5-b]pyridin-3-yl)benzyl)-2,6-dimethylpiperazin-1-yl)-4-methoxycyclobut-3-ene-1,2-dione

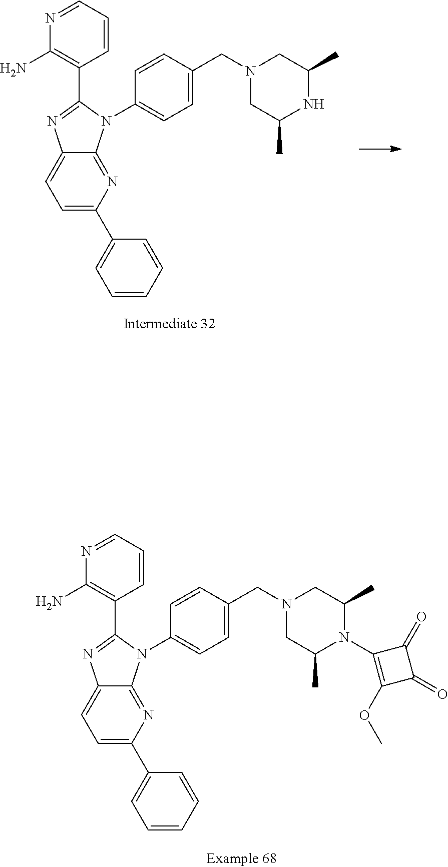 [SMILES: C[C@@H]1CN(Cc2ccc(-n3c(-c4cccnc4N)nc4ccc(-c5ccccc5)nc43)cc2)C[C@H](C)N1 ; COc1c(N2[C@H](C)CN(Cc3ccc(-n4c(-c5cccnc5N)nc5ccc(-c6ccccc6)nc54)cc3)C[C@@H]2C)c(=O)c1=O]

To a solution of Intermediate 32 (80 mg, 163 μmol), 3,4-dimethoxycyclobut-3-ene-1,2-dione (27.9 mg, 196 μmol) in MeOH (1 mL) was added TEA (82.7 mg, 817 mol). The mixture was stirred at 25° C. for 2 hr. The mixture was filtered and concentrated to give 3-((2S,6R)-4-(4-(2-(2-aminopyridin-3-yl)-5-phenyl-3H-imidazo[4,5-b]pyridin-3-yl)benzyl)-2,6-dimethylpiperazin-1-yl)-4-methoxycyclobut-3-ene-1,2-dione (Example 68, 12.8 mg, yield: 13%) as yellow solid. MS: m/z=600.2 [M+H]⁺. ¹H NMR (400 MHz, Dimethylsulfoxide-d₆) δ 8.27 (d, J=8.4 Hz, 1H), 7.98-8.05 (m, 4H), 7.56 (d, J=8.4 Hz, 2H), 7.43-7.49 (m, 4H), 7.37-7.42 (m, 1H), 7.17 (dd, J=7.6, 2.0 Hz, 1H), 7.04 (br s, 2H), 6.39 (dd, J=7.6, 4.8 Hz, 1H), 4.31 (s, 3H), 3.63 (s, 2H), 3.34-3.40 (m, 2H), 2.72-2.74 (m, 2H), 2.34-2.38 (m, 2H), 1.38 (d, J=7.2 Hz, 6H).

### Example 69: 3-((2R,6R)-4-(4-(2-(2-Aminopyridin-3-yl)-5-phenyl-3H-imidazo[4,5-b]pyridin-3-yl)benzyl)-2,6-dimethylpiperazin-1-yl)-4-methoxycyclobut-3-ene-1,2-dione

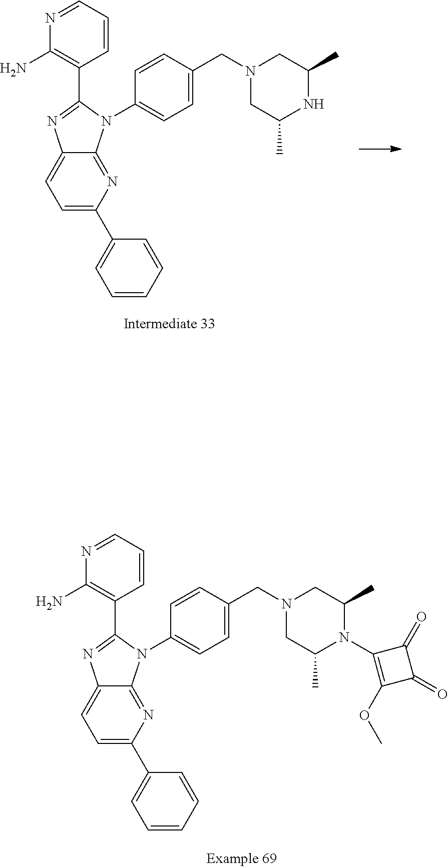 [SMILES: C[C@@H]1CN(Cc2ccc(-n3c(-c4cccnc4N)nc4ccc(-c5ccccc5)nc43)cc2)C[C@@H](C)N1 ; COc1c(N2[C@H](C)CN(Cc3ccc(-n4c(-c5cccnc5N)nc5ccc(-c6ccccc6)nc54)cc3)C[C@H]2C)c(=O)c1=O]

To a solution of Intermediate 33 (66 mg, 135 μmol) and TEA (68.2 mg, 674 μmol) in MeOH (2 mL) was added 3,4-dimethoxycyclobut-3-ene-1,2-dione (23 mg, 162 μmol). The mixture was stirred at 25° C. for 16 hr. The mixture was concentrated and purified by silica gel flash chromatography (Eluent of 0~8% MeOH in CH₂Cl₂) to give 3-((2R,6R)-4-(4-(2-(2-aminopyridin-3-yl)-5-phenyl-3H-imidazo[4,5-b]pyridin-3-yl)benzyl)-2,6-dimethylpiperazin-1-yl)-4-methoxycyclobut-3-ene-1,2-dione (Example 69, 44.6 mg, yield: 54%) as a yellow solid. MS: m/z=600.3 [M+H]⁺. ¹H NMR (400 MHz, Dimethylsulfoxide-d₆) δ 8.27 (d, J=8.4 Hz, 1H), 8.10-7.94 (m, 4H), 7.54-7.37 (m, 7H), 7.17 (dd, J=7.6, 1.6 Hz, 1H), 7.03 (br s, 2H), 6.38 (dd, J=7.6, 4.8 Hz, 1H), 4.34 (s, 3H), 4.30-4.18 (m, 2H), 3.67 (d, J=13.6 Hz, 1H), 3.56 (d, J=13.6 Hz, 1H), 2.68-2.63 (m, 2H), 2.36-2.30 (m, 2H), 1.35 (d, J=6.4 Hz, 6H).

### Example 70: 3-((2S,6S)-4-(4-(2-(2-Aminopyridin-3-yl)-5-phenyl-3H-imidazo[4,5-b]pyridin-3-yl)benzyl)-2,6-dimethylpiperazin-1-yl)-4-methoxycyclobut-3-ene-1,2-dione

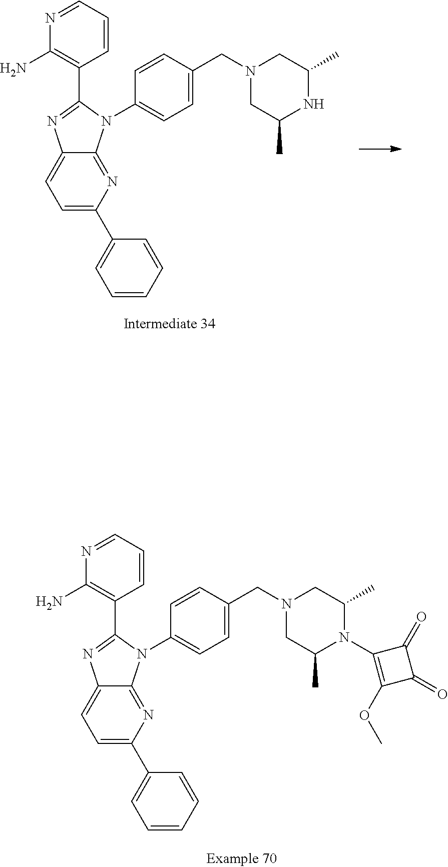 [SMILES: C[C@H]1CN(Cc2ccc(-n3c(-c4cccnc4N)nc4ccc(-c5ccccc5)nc43)cc2)C[C@H](C)N1 ; COc1c(N2[C@@H](C)CN(Cc3ccc(-n4c(-c5cccnc5N)nc5ccc(-c6ccccc6)nc54)cc3)C[C@@H]2C)c(=O)c1=O]

To a solution of Intermediate 34 (80 mg, 163 μmol) in MeOH (1 mL) were added TEA (82.7 mg, 817 mol) and 3,4-dimethoxycyclobut-3-ene-1,2-dione (27.9 mg, 196 μmol). The reaction mixture stirred at 25° C. for 16 hr. The reaction mixture was filtrated, washed with MeOH (10 ml×2), concentrated under reduced pressure. After purified by silica gel flash chromatography (Eluent of 0-5% MeOH in CH₂C2), 3-((2S,6S)-4-(4-(2-(2-aminopyridin-3-yl)-5-phenyl-3H-imidazo[4,5-b]pyridin-3-yl)benzyl)-2,6-dimethylpiperazin-1-yl)-4-methoxycyclobut-3-ene-1,2-dione (Example 70, 5.5 mg, yield: 5.4%) was obtained as a yellow solid. MS: m/z=600.3 [M+H]⁺. ¹H NMR (400 MHz, Methanol-d₄) δ 8.19 (d, J=8.8 Hz, 1H), 8.05-8.01 (m, 2H), 7.98 (dd, J=4.8, 1.6 Hz, 1H), 7.94 (d, J=8.4 Hz, 1H), 7.54 (d, J=8.0 Hz, 2H), 7.47-7.35 (m, 5H), 7.32 (dd, J=7.6, 1.6 Hz, 1H), 6.47 (dd, J=7.6, 5.2 Hz, 1H), 4.41 (s, 3H), 4.40-4.32 (m, 2H), 3.68 (d, J=13.6 Hz, 1H), 3.68 (d, J=13.6 Hz, 1H), 2.67 (dd, J=11.6, 3.2 Hz, 2H), 2.43-2.35 (m, 2H), 1.43 (d, J=6.8 Hz, 6H).

### Example 71: 3-(8-(4-(2-(2-Aminopyridin-3-yl)-5-phenyl-3H-imidazo[4,5-b]pyridin-3-yl)benzyl)-3,8-diazabicyclo[3.2.1]octan-3-yl)-4-methoxycyclobut-3-ene-1,2-dione

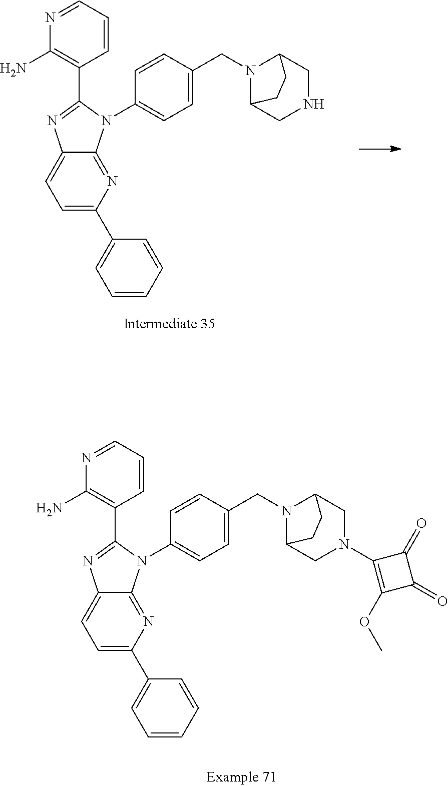 [SMILES: Nc1ncccc1-c1nc2ccc(-c3ccccc3)nc2n1-c1ccc(CN2C3CCC2CNC3)cc1 ; COc1c(N2CC3CCC(C2)N3Cc2ccc(-n3c(-c4cccnc4N)nc4ccc(-c5ccccc5)nc43)cc2)c(=O)c1=O]

To a solution of 3,4-dimethoxycyclobut-3-ene-1,2-dione (32.0 mg, 225 μmol) and Intermediate 35 (100 mg, 205 mol) in MeOH (2 mL) was added TEA (103 mg, 1.03 mmol) at 0° C. The mixture was stirred at 25° C. for 2 hr. The reaction was filtered and concentrated under reduced pressure. After purified by prep-HPLC (column: Waters xbridge 150×25 mm×10 μm; mobile phase: [water (NH₄HCO₃)-ACN]; B %: 40%-70%, 10 min), 3-(8-(4-(2-(2-aminopyridin-3-yl)-5-phenyl-3H-imidazo[4,5-b]pyridin-3-yl)benzyl)-3,8-diazabicyclo[3.2.1]octan-3-yl)-4-methoxycyclobut-3-ene-1,2-dione (Example 71, 16 mg, yield: 13%) was obtained as a light yellow solid. MS: m/z=598.3 [M+H]⁺. ¹H NMR (400 MHz, Dimethylsulfoxide-d₆) δ 8.26 (d, J=8.4 Hz, 1H), 8.05-7.98 (m, 4H), 7.56 (d, J=8.0 Hz, 2H), 7.50-7.36 (m, 6H), 7.17 (dd, J=7.6, 1.6 Hz, 1H), 6.99 (d, J=4.4 Hz, 1H), 6.40 (dd, J=7.6, 4.0 Hz, 1H), 4.29 (s, 3H), 4.13-4.06 (m, 1H), 3.64 (s, 2H), 3.55-3.45 (m, 3H), 3.30-3.28 (m, 2H), 2.10-2.05 (m, 2H), 1.65-1.60 (m, 2H).

### Example 72: 3-(3-(4-(2-(2-Aminopyridin-3-yl)-5-phenyl-3H-imidazo[4,5-b]pyridin-3-yl)benzyl)-3,8-diazabicyclo[3.2.1]octan-8-yl)-4-methoxycyclobut-3-ene-1,2-dione

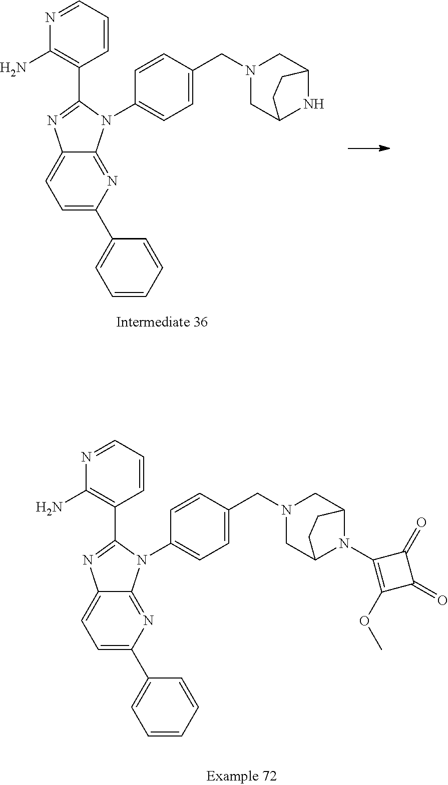 [SMILES: Nc1ncccc1-c1nc2ccc(-c3ccccc3)nc2n1-c1ccc(CN2CC3CCC(C2)N3)cc1 ; COc1c(N2C3CCC2CN(Cc2ccc(-n4c(-c5cccnc5N)nc5ccc(-c6ccccc6)nc54)cc2)C3)c(=O)c1=O]

To a solution of Intermediate 36 (100 mg, 205 mol) and 3,4-dimethoxycyclobut-3-ene-1,2-dione (32.1 mg, 226 μmol) in MeOH (2 mL) was added TEA (104 mg, 1.03 mmol) at 0° C. The mixture was stirred at 25° C. for 2 hr. The reaction mixture was filtered and concentrated under reduced pressure. After purified by prep-TLC (CH₂Cl₂:MeOH=10:1), 3-(3-(4-(2-(2-aminopyridin-3-yl)-5-phenyl-3H-imidazo[4,5-b]pyridin-3-yl)benzyl)-3,8-diazabicyclo[3.2.1]octan-8-yl)-4-methoxycyclobut-3-ene-1,2-dione (Example 72, 29.2 mg, yield: 24%) was obtained as a light yellow solid. MS: m/z=598.3 [M+H]⁺. ¹H NMR (400 MHz, Dimethylsulfoxide-d₆) δ 8.27 (d, J=8.4 Hz, 1H), 8.05-7.98 (m, 4H), 7.50-7.36 (m, 7H), 7.13 (dd, J=7.6, 1.6 Hz, 1H), 7.03 (s, 2H), 6.36 (dd, J=7.6, 4.0 Hz, 1H), 4.65-4.55 (m, 1H), 4.31 (s, 3H), 4.25-4.17 (m, 1H), 3.61 (s, 2H), 2.80-2.75 (m, 2H), 2.35-2.30 (m, 2H), 1.99-1.87 (m, 4H).

### Example 73: 3-((1-(4-(2-(2-Aminopyridin-3-yl)-5-phenyl-3H-imidazo[4,5-b]pyridin-3-yl)benzyl)azepan-4-yl)amino)-4-methoxycyclobut-3-ene-1,2-dione

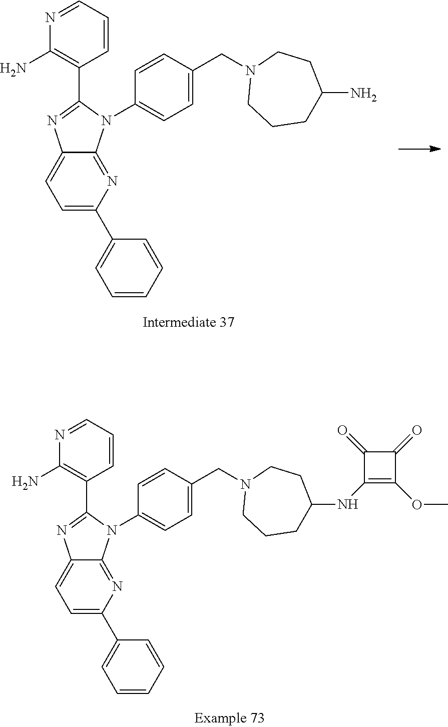 [SMILES: Nc1ncccc1-c1nc2ccc(-c3ccccc3)nc2n1-c1ccc(CN2CCCC(N)CC2)cc1 ; COc1c(NC2CCCN(Cc3ccc(-n4c(-c5cccnc5N)nc5ccc(-c6ccccc6)nc54)cc3)CC2)c(=O)c1=O]

To a solution of Intermediate 37 (60 mg, 114 μmol) and TEA (57.7 mg, 570 μmol) in MeOH (1 mL) was added 3,4-dimethoxycyclobut-3-ene-1,2-dione (19.5 mg, 136.9 μmol). The mixture was stirred at 25° C. for 16 hr. The mixture was concentrated to give a residue. The residue was purified by silica gel flash chromatography (Eluent of 0~10% MeOH in CH₂Cl₂) to give 3-((1-(4-(2-(2-aminopyridin-3-yl)-5-phenyl-3H-imidazo[4,5-b]pyridin-3-yl)benzyl)azepan-4-yl)amino)-4-methoxycyclobut-3-ene-1,2-dione (Example 73, 18.5 mg, yield: 26%, 6:4 mixture of tautomers) as a yellow solid. MS: m/z=600.3 [M+H]⁺. ¹H NMR (400 MHz, Dimethylsulfoxide-d₆) δ 8.86 (d, J=7.2 Hz, 0.6H), 8.63 (m, J=7.2 Hz, 0.4H), 8.27 (d, J=8.4 Hz, 1H), 8.04-7.97 (m, 4H), 7.52-7.35 (m, 7H), 7.15 (d, J=7.2 Hz, 1H), 7.04 (br s, 2H), 6.37 (dd, J=7.6, 4.8 Hz, 1H), 4.28 (s, 3H), 3.86-3.73 (m, 1H), 3.70 (s, 2H), 2.71-2.58 (m, 4H), 1.94-1.84 (m, 2H), 1.76-1.71 (m, 2H), 1.64-1.49 (m, 2H).

### Example 74: 3-((1-(4-(2-(2-Aminopyridin-3-yl)-5-phenyl-3H-imidazo[4,5-b]pyridin-3-yl)benzyl)azepan-3-yl)amino)-4-methoxycyclobut-3-ene-1,2-dione

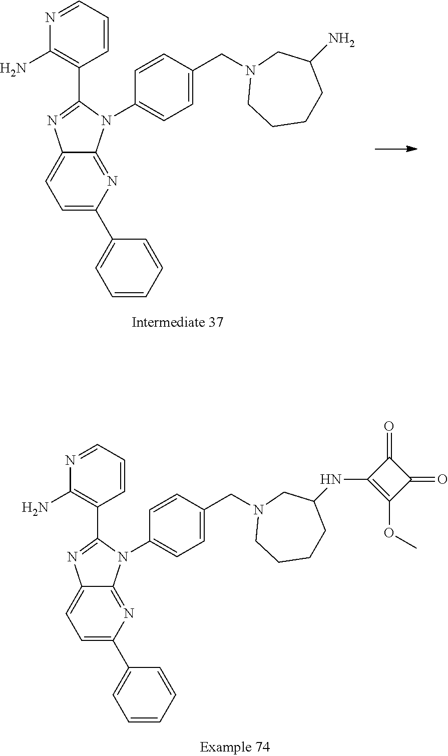 [SMILES: Nc1ncccc1-c1nc2ccc(-c3ccccc3)nc2n1-c1ccc(CN2CCCCC(N)C2)cc1 ; COc1c(NC2CCCCN(Cc3ccc(-n4c(-c5cccnc5N)nc5ccc(-c6ccccc6)nc54)cc3)C2)c(=O)c1=O]

To a solution of Intermediate 38 (80.0 mg, 164 μmol) and TEA (83.0 mg, 570 μmol) in MeOH (1 mL) was added 3,4-dimethoxycyclobut-3-ene-1,2-dione (23.2 mg, 164 mol). The mixture was stirred at 25° C. for 16 hr. The mixture was concentrated and purified by silica gel flash chromatography (Eluent of 2~3% MeOH in CH₂Cl₂), to give 3-((1-(4-(2-(2-aminopyridin-3-yl)-5-phenyl-3H-imidazo[4,5-b]pyridin-3-yl)benzyl)azepan-3-yl)amino)-4-methoxycyclobut-3-ene-1,2-dione (Example 74, 17.2 mg, yield: 16%, 6:4 mixture of tautomers) as a yellow solid. MS: m/z=600.3 [M+H]⁺. ¹H NMR (400 MHz, Dimethylsulfoxide-d₆) δ 8.75 (d, J=8.0 Hz, 0.6H), 8.56 (d, J=8.0 Hz, 0.4H), 8.26 (d, J=8.4 Hz, 1H), 8.04-7.96 (m, 4H), 7.52 (d, J=8.4 Hz, 2H), 7.48-7.42 (m, 4H), 7.41-7.37 (m, 1H), 7.11 (dd, J=7.6, 2.0 Hz, 1H), 7.04 (br s, 2H), 6.35-6.29 (m, 1H), 4.25-4.19 (m, 3H), 4.18-4.14 (m, 0.4H), 3.746 (s, 2H), 3.75-3.71 (m, 0.6H), 2.93-2.84 (m, 1H), 2.71-2.62 (m, 3H), 1.96-1.83 (m, 1H), 1.73-1.63 (m, 3H), 1.58-1.47 (m, 2H).

### Example 75: 3-((1 S,4S)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)-4-methoxycyclobut-3-ene-1,2-dione

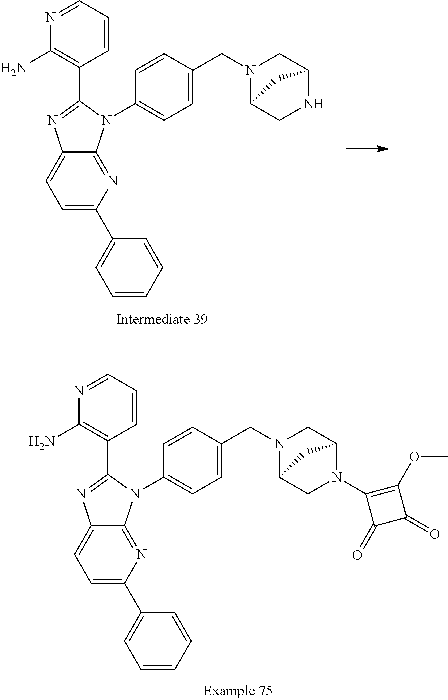 [SMILES: Nc1ncccc1-c1nc2ccc(-c3ccccc3)nc2n1-c1ccc(CN2C[C@@H]3C[C@H]2CN3)cc1 ; COc1c(N2C[C@@H]3C[C@H]2CN3Cc2ccc(-n3c(-c4cccnc4N)nc4ccc(-c5ccccc5)nc43)cc2)c(=O)c1=O]

To a solution of Intermediate 39 (150 mg, 317 μmol) and 3,4-dimethoxycyclobut-3-ene-1,2-dione (45 mg, 317 μmol) in MeOH (1 mL) was added TEA (132 μL, 950 μmol). The mixture was degassed and purged with N₂ three times and stirred at 25° C. for 16 hr under N₂. The reaction mixture was filtered and concentrated under reduced pressure. The residue was purified by prep-TLC (CH₂C₂:MeOH=10:1) to give 3-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-4-methoxycyclobut-3-ene-1,2-dione (Example 75, 41 mg, yield: 22%) as a yellow solid. MS: m/z=584.2 [M+H]⁺. ¹H NMR (400 MHz, Methanol-d₄) δ 8.19 (d, J=8.4 Hz, 1H), 8.03 (d, J=7.2 Hz, 2H), 7.98 (dd, J=5.2, 1.6 Hz, 1H), 7.94 (d, J=8.4 Hz, 1H), 7.57 (d, J=8.4 Hz, 2H), 7.47-7.36 (m, 5H), 7.33 (d, J=7.6 Hz, 1H), 6.48 (dd, J=7.2, 4.8 Hz, 1H), 4.41-4.34 (m, 3H), 4.13-3.94 (m, 1H), 3.94-3.77 (m, 3H), 3.76-3.70 (m, 1H), 3.69-3.55 (m, 1H), 3.03-2.93 (m, 1H), 2.83-2.75 (m, 1H), 2.18-2.07 (m, 1H), 1.93-1.83 (m, 1H).

### Example 76: 3-((1R,4R)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)-4-methoxycyclobut-3-ene-1,2-dione

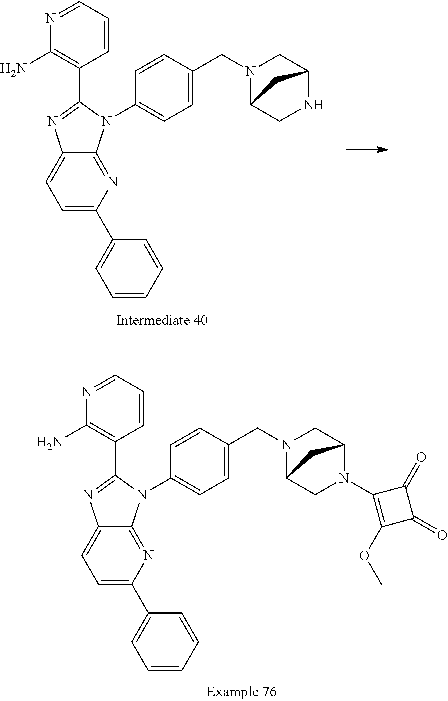 [SMILES: Nc1ncccc1-c1nc2ccc(-c3ccccc3)nc2n1-c1ccc(CN2C[C@H]3C[C@@H]2CN3)cc1 ; COc1c(N2C[C@H]3C[C@@H]2CN3Cc2ccc(-n3c(-c4cccnc4N)nc4ccc(-c5ccccc5)nc43)cc2)c(=O)c1=O]

To a solution of Intermediate 40 (50 mg, 105 μmol) and 3,4-dimethoxycyclobut-3-ene-1,2-dione (18.0 mg, 126 μmol) in MeOH (1 mL) was added TEA (53.4 mg, 528 μmol). The mixture was stirred at 25° C. for 16 hr. The reaction was filtered and concentrated under reduced pressure. The residue was purified by silica gel flash chromatography (Eluent of 2~3% MeOH in CH₂Cl₂) to give 3-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-4-methoxycyclobut-3-ene-1,2-dione (Example 76, 16.1 mg, yield: 26%) as a yellow solid. MS: m/z=584.3 [M+H]⁺. ¹H NMR (400 MHz, Methanol-d₄) δ 8.19 (d, J=8.4 Hz, 1H), 8.03 (d, J=7.2 Hz, 2H), 7.98 (dd, J=5.2, 2.0 Hz, 1H), 7.94 (d, J=8.4 Hz, 1H), 7.56 (d, J=8.4 Hz, 2H), 7.45-7.41 (m, 4H), 7.39-7.37 (m, 1H), 7.33-7.31 (m, 1H), 6.48 (dd, J=7.6, 5.2 Hz, 1H), 4.60 (s, 2H), 4.39-4.35 (m, 3H), 3.92-3.84 (m, 2H), 3.71-3.65 (m, 2H), 2.96 (d, J=10.0 Hz, 1H), 2.81-2.77 (m, 1H), 2.13-2.11 (m, 1H), 1.89-1.85 (m, 1H).

### Example 77: 3-(4-((4-(2-(2-Aminopyridin-3-yl)-5-phenyl-3H-imidazo[4,5-b]pyridin-3-yl)benzyl)amino)piperidin-1-yl)-4-methoxycyclobut-3-ene-1,2-dione

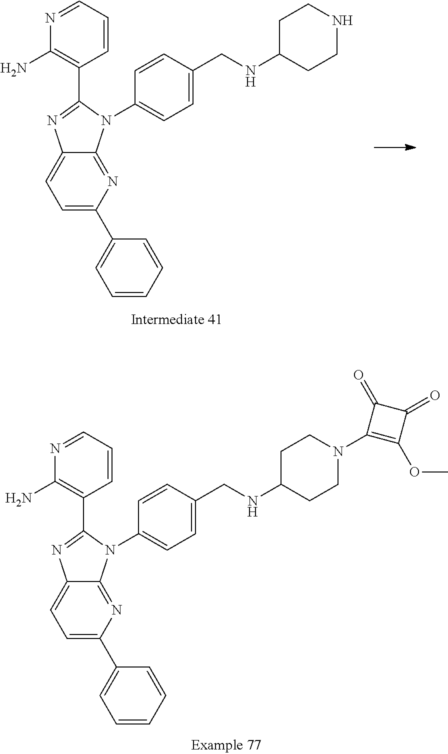 [SMILES: Nc1ncccc1-c1nc2ccc(-c3ccccc3)nc2n1-c1ccc(CNC2CCNCC2)cc1 ; COc1c(N2CCC(NCc3ccc(-n4c(-c5cccnc5N)nc5ccc(-c6ccccc6)nc54)cc3)CC2)c(=O)c1=O]

To a solution of Intermediate 41 (80 mg, 156 μmol) and 3,4-dimethoxycyclobut-3-ene-1,2-dione (26.6 mg, 187 μmol) in MeOH (2 mL) was added TEA (79.1 mg, 781 mol). The mixture was stirred at 25° C. for 1 hr. The reaction mixture was concentrated under reduced pressure. After purified by prep-HPLC (column: Waters xbridge 150×25 mm×10 μm; mobile phase: [water (NH₄HCO₃)-ACN]; gradient: 30%-60% B over 14 min), 3-(4-((4-(2-(2-aminopyridin-3-yl)-5-phenyl-3H-imidazo[4,5-b]pyridin-3-yl)benzyl)amino)piperidin-1-yl)-4-methoxycyclobut-3-ene-1,2-dione (Example 77, 8 mg, yield: 8.5%) was obtained as a light-yellow solid. MS: m/z=586.2 [M+H]⁺. ¹H NMR (400 MHz, Methanol-d₄) δ 8.19 (d, J=8.4 Hz, 1H), 8.04-7.92 (m, 4H), 7.58 (d, J=8.4 Hz, 2H), 7.48-7.34 (m, 6H), 6.48 (dd, J=7.6, 5.2 Hz, 1H), 4.58-4.54 (m, 1H), 4.38 (s, 3H), 4.09-4.00 (m, 1H), 3.94 (s, 2H), 2.97-2.83 (m, 1H), 2.15-2.04 (m, 2H), 1.69-1.46 (m, 4H).

### Example 78: 3-(4-((4-(2-(2-Aminopyridin-3-yl)-5-phenyl-3H-imidazo[4,5-b]pyridin-3-yl)benzyl)oxy)piperidin-1-yl)-4-methoxycyclobut-3-ene-1,2-dione

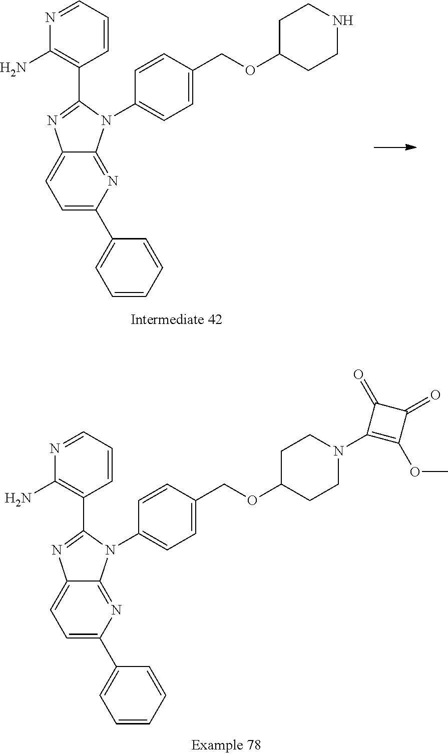 [SMILES: Nc1ncccc1-c1nc2ccc(-c3ccccc3)nc2n1-c1ccc(COC2CCNCC2)cc1 ; COc1c(N2CCC(OCc3ccc(-n4c(-c5cccnc5N)nc5ccc(-c6ccccc6)nc54)cc3)CC2)c(=O)c1=O]

To a solution of Intermediate 42 (200 mg, 420 mol) and 3,4-dimethoxycyclobut-3-ene-1,2-dione (72 mg, 504 mol) in MeOH (5 mL) was added TEA (85 mg, 839 μmol). The mixture was stirred at 25° C. for 16 hr. The reaction mixture was concentrated under reduced pressure. After purified by prep-TLC (MeOH in CH₂Cl₂=10%), 3-(4-((4-(2-(2-aminopyridin-3-yl)-5-phenyl-3H-imidazo[4,5-b]pyridin-3-yl)benzyl)oxy)piperidin-1-yl)-4-methoxycyclobut-3-ene-1,2-dione (Example 78, 10.1 mg, yield: 4%) was obtained as a light-yellow solid. MS: m/z=587.1 [M+H]⁺. ¹H NMR (400 MHz, Dimethylsulfoxide-d₆) δ 8.21-8.17 (m, 1H), 8.04 (d, J=7.6 Hz, 2H), 7.99 (dd, J=5.2, 1.6 Hz, 1H), 7.95 (d, J=8.4 Hz, 1H), 7.57 (d, J=8.4 Hz, 2H), 7.48-7.40 (m, 4H), 7.39-7.33 (m, 2H), 6.49 (dd, J=7.6, 4.8 Hz, 1H), 4.70 (s, 2H), 4.15-4.01 (m, 1H), 3.89-3.78 (m, 3H), 3.66-3.52 (m, 2H), 2.05-2.02 (m, 2H), 1.92-1.78 (m, 2H), 1.34-1.32 (m, 2H).

### Example 79: (R)-3-(4-(4-(2-(2-Aminopyridin-3-yl)-5-phenyl-3H-imidazo[4,5-b]pyridin-3-yl)benzyl)-2-(hydroxymethyl)piperazin-1-yl)-4-methoxycyclobut-3-ene-1,2-dione

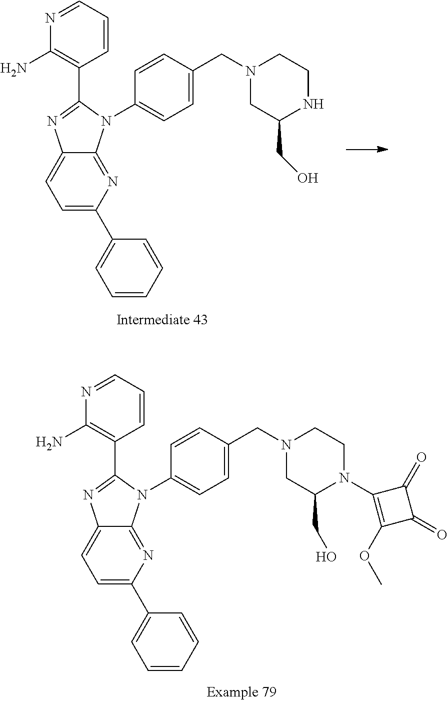 [SMILES: Nc1ncccc1-c1nc2ccc(-c3ccccc3)nc2n1-c1ccc(CN2CCN[C@@H](CO)C2)cc1 ; COc1c(N2CCN(Cc3ccc(-n4c(-c5cccnc5N)nc5ccc(-c6ccccc6)nc54)cc3)C[C@@H]2CO)c(=O)c1=O]

To a solution of Intermediate 43 (70 mg, 142 μmol) and 3,4-dimethoxycyclobut-3-ene-1,2-dione (22.3 mg, 157 μmol) in MeOH (2 mL) was added TEA (72.0 mg, 712 gmol). The mixture was stirred at 25° C. for 16 hr. The mixture filtered and concentrated under reduced pressure. After purified by silica gel flash chromatography (MeOH in CH₂Cl₂=0-10%), (R)-3-(4-(4-(2-(2-Aminopyridin-3-yl)-5-phenyl-3H-imidazo[4,5-b]pyridin-3-yl)benzyl)-2-(hydroxymethyl)piperazin-1-yl)-4-methoxycyclobut-3-ene-1,2-dione (Example 79, 25 mg, yield: 29%) was obtained as a yellow solid. MS: m/z=602.2 [M+H]⁺. ¹H NMR (400 MHz, Dimethylsulfoxide-d₆) δ 8.27 (d, J=8.4 Hz, 1H), 8.05-7.98 (m, 4H), 7.50-7.36 (m, 7H), 7.16 (dd, J=7.6, 1.6 Hz, 1H), 7.03 (s, 2H), 6.38 (dd, J=7.6, 4.0 Hz, 1H), 5.00-4.92 (m, 11H), 4.50-4.40 (m, 1H), 4.29 (s, 3H), 4.10 (m, 1H), 4.00-3.84 (m, 2H), 3.65-3.53 (m, 3H), 2.92-2.80 (m, 2H), 2.33-2.20 (m, 2H).

### Example 80: (S)-3-(4-(4-(2-(2-Aminopyridin-3-yl)-5-phenyl-3H-imidazo[4,5-b]pyridin-3-yl)benzyl)-2-(hydroxymethyl)piperazin-1-yl)-4-methoxycyclobut-3-ene-1,2-dione

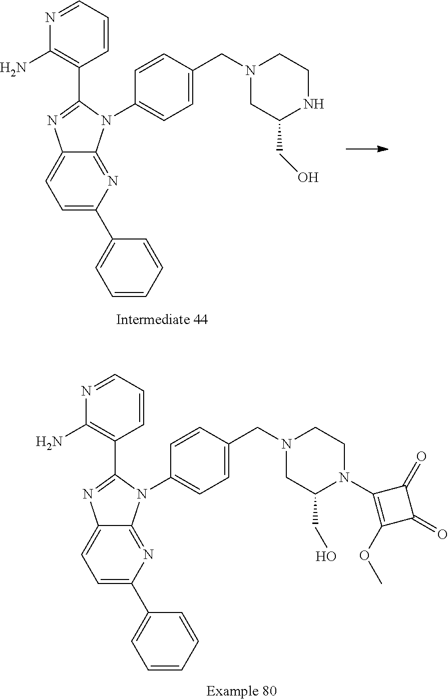 [SMILES: Nc1ncccc1-c1nc2ccc(-c3ccccc3)nc2n1-c1ccc(CN2CCN[C@H](CO)C2)cc1 ; COc1c(N2CCN(Cc3ccc(-n4c(-c5cccnc5N)nc5ccc(-c6ccccc6)nc54)cc3)C[C@H]2CO)c(=O)c1=O]

To a solution of Intermediate 44 (50 mg, 102 μmol) in MeOH (1 mL) were added TEA (51.5 mg, 509 mol) and 3,4-dimethoxycyclobut-3-ene-1,2-dione (17.3 mg, 122 μmol). The reaction mixture was stirred at 25° C. for 16 hr. The mixture was filtered. The collected solid was washed with MeOH (2 mL×2) to give (S)-3-(4-(4-(2-(2-aminopyridin-3-yl)-5-phenyl-3H-imidazo[4,5-b]pyridin-3-yl)benzyl)-2-(hydroxymethyl)piperazin-1-yl)-4-methoxycyclobut-3-ene-1,2-dione (Example 80, 19.5 mg, yield: 31%) as a light yellow solid. MS: m/z=602.3 [M+H]⁺. ¹H NMR (400 MHz, Dimethylsulfoxide-d₆) δ 8.27 (d, J=8.4 Hz, 1H), 8.05-7.98 (m, 4H), 7.50-7.44 (m, 6H), 7.42-7.37 (m, 1H), 7.16 (dd, J=7.6, 2.0 Hz, 1H), 7.03 (s, 2H), 6.40 (dd, J=7.6, 4.8 Hz, 1H), 5.02-4.86 (m, 1H), 4.48-4.41 (m, 0.5H), 4.30 (s, 3H), 4.01-3.87 (m, 1.5H), 3.66-3.61 (m, 1H), 3.56-3.52 (m, 2H), 3.36-3.34 (m, 2H), 2.92-2.82 (m, 2H), 2.32-2.20 (m, 2H).

### Example 81: (S)-3-((1-(4-(2-(2-Aminopyridin-3-yl)-5-phenyl-3H-imidazo[4,5-b]pyridin-3-yl)benzyl)pyrrolidin-3-yl)(methyl)amino)-4-methoxycyclobut-3-ene-1,2-dione

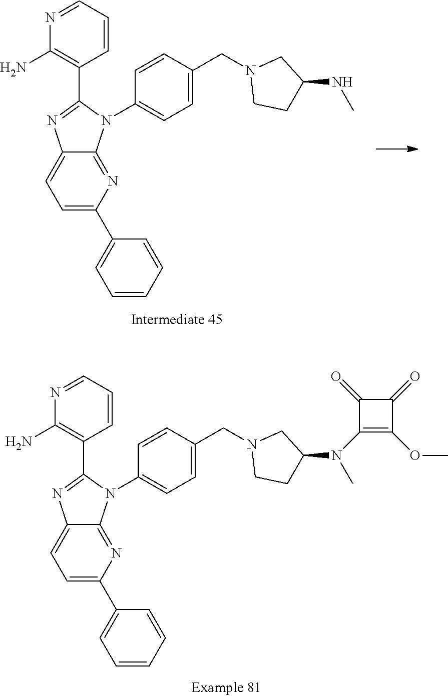 [SMILES: CN[C@H]1CCN(Cc2ccc(-n3c(-c4cccnc4N)nc4ccc(-c5ccccc5)nc43)cc2)C1 ; COc1c(N(C)[C@H]2CCN(Cc3ccc(-n4c(-c5cccnc5N)nc5ccc(-c6ccccc6)nc54)cc3)C2)c(=O)c1=O]

To a solution of Intermediate 45 (50 mg, 105 μmol) in MeOH (2 mL) were added 3,4-dimethoxycyclobut-3-ene-1,2-dione (14.9 mg, 105 μmol) and TEA (23.4 mg, 231 μmol). The reaction mixture was stirred at 25° C. for 2 hr. The mixture was concentrated under reduced pressure. The residue was purified by prep-TLC (CH₂Cl₂:MeOH=10:1) to give (S)-3-((1-(4-(2-(2-Aminopyridin-3-yl)-5-phenyl-3H-imidazo[4,5-b]pyridin-3-yl)benzyl)pyrrolidin-3-yl)(methyl)amino)-4-methoxycyclobut-3-ene-1,2-dione (Example 81, 7.1 mg, yield: 11%) as a light-yellow solid. MS: m/z=586.3 [M+H]⁺. ¹H NMR (400 MHz, Methanol-d₄) δ 8.18 (d, J=8.4 Hz, 1H), 8.05-8.00 (m, 2H), 7.97 (dd, J=4.8, 1.6 Hz, 1H), 7.93 (d, J=8.4 Hz, 1H), 7.53 (d, J=8.4 Hz, 2H), 7.45-7.35 (m, 5H), 7.31 (dd, J=7.6, 2.0 Hz, 1H), 6.46 (dd, J=7.6, 5.2 Hz, 1H), 4.35 (s, 3H), 3.83-3.76 (m, 1H), 3.34 (br s, 3H), 3.19 (s, 2H), 3.01-2.94 (m, 1H), 2.89-2.86 (m, 1H), 2.66-2.57 (m, 1H), 2.49-2.39 (m, 1H), 2.26-2.16 (m, 1H), 2.05-1.99 (m, 1H).

### Example 82: 6-(1-(4-(2-(2-Aminopyridin-3-yl)-5-phenyl-3H-imidazo[4,5-b]pyridin-3-yl)benzyl)pyrrolidin-3-yl)-2-oxa-6-azabicyclo[5.2.0]non-1(7)-ene-8,9-dione

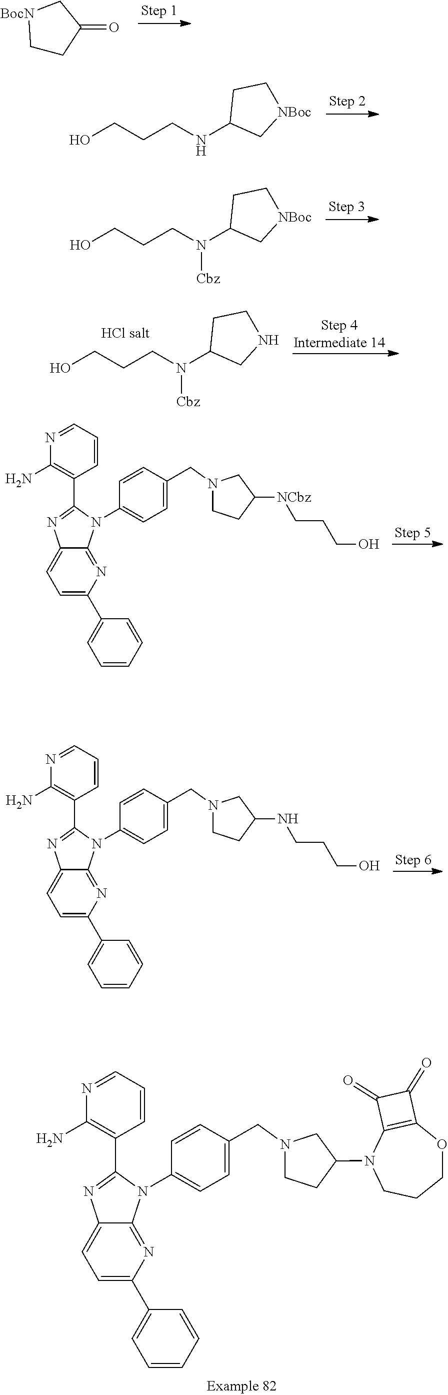

### Step 1: tert-Butyl 3-((3-hydroxypropyl)amino)pyrrolidine-1-carboxylate

To a solution of tert-butyl 3-oxopyrrolidine-1-carboxylate (20 g, 108 mmol) and 3-aminopropan-1-ol (16.2 g, 215 mmol) in CH₂Cl₂ (200 mL) was added AcOH (6.48 g, 108 mmol). The resulting mixture was degassed and purged with N₂ three times. NaBH(OAc)₃ was added (45.7 g, 216 mmol). The mixture was stirred at 25° C. for 12 hr. The pH of the reaction mixture was adjusted to 9-10 with saturated aqueous NaOH. The mixture was extracted with CH₂Cl₂ (200 mL×2). The combined organic layers were washed with brine, dried over Na₂SO₄, filtered and concentrated under reduced pressure. tert-Butyl 3-((3-hydroxypropyl)amino)pyrrolidine-1-carboxylate (23 g, yield: 87%) was obtained as a black brown oil, which was used in the next step without further purification. ¹H NMR (400 MHz, Chloroform-d) δ 3.87-3.73 (m, 2H), 3.61-3.52 (m, 1H), 3.50-3.25 (m, 3H), 3.21-3.01 (m, 1H), 2.96-2.80 (m, 2H), 2.55-2.46 (m, 2H), 2.13-1.98 (m, 1H), 1.80-1.63 (m, 3H), 1.46 (s, 9H).

### Step 2: tert-Butyl 3-(((benzyloxy)carbonyl)(3-hydroxypropyl)amino)pyrrolidine-1-carboxylate

To a solution of tert-butyl 3-((3-hydroxypropyl)amino)pyrrolidine-1-carboxylate (5.0 g, 20.5 mmol) in CH₂Cl₂ (50 mL) at 0° C. were added TEA (4.14 g, 40.9 mmol) and CbzCl (3.84 g, 22.5 mmol). The mixture was stirred at 25° C. for 12 hr. The reaction mixture was diluted with H₂O (20 mL) and extracted with EtOAc (30 mL×3). The combined organic layers were washed with brine (30 mL), dried over Na₂SO₄, filtered and concentrated under reduced pressure. tert-Butyl 3-(((benzyloxy)carbonyl)(3-hydroxypropyl)amino)pyrrolidine-1-carboxylate (7.7 g, crude) was obtained as a black brown oil.

### Step 3: Benzyl (3-hydroxypropyl)(pyrrolidin-3-yl)carbamate

To a solution of tert-butyl 3-(((benzyloxy)carbonyl)(3-hydroxypropyl)amino)pyrrolidine-1-carboxylate (7.7 g, 16.3 mmol,) in CH₂Cl₂ (20 mL) was added HCl in 1,4-dioxane (4 M, 4.07 mL). The mixture was stirred at 25° C. for 2 hr. The mixture was concentrated under reduced pressure to give benzyl (3-hydroxypropyl)(pyrrolidin-3-yl)carbamate (4.5 g, HCl salt) as black brown oil.

### Step 4: Benzyl (1-(4-(2-(2-aminopyridin-3-yl)-5-phenyl-3H-imidazo[4,5-b]pyridin-3-yl)benzyl)pyrrolidin-3-yl)(3-hydroxypropyl)carbamate

To a solution of Intermediate 14 (500 mg, 1.21 mmol) and benzyl (3-hydroxypropyl)(pyrrolidin-3-yl)carbamate (459 mg, 1.46 mmol, HCl salt) in DMF (10 mL) were added NaI (18.2 mg, 121 μmol) and K₂CO₃ (839 mg, 6.07 mmol). The mixture was stirred at 25° C. for 3 hr. The reaction mixture was diluted with H₂O (10 mL) and extracted with EtOAc (10 mL×2). The combined organic layers were washed with brine (10 mL), dried over Na₂SO₄, filtered, and concentrated under reduced pressure. After purified by silica gel flash chromatography (MeOH in CH₂Cl₂=0% to 8%), benzyl (1-(4-(2-(2-aminopyridin-3-yl)-5-phenyl-3H-imidazo[4,5-b]pyridin-3-yl)benzyl)pyrrolidin-3-yl)(3-hydroxypropyl)carbamate (230 mg, yield: 24%) was obtained as a yellow oil. MS: m/z=654.5 [M+H]⁺.

### Step 5: 3-((1-(4-(2-(2-Aminopyridin-3-yl)-5-phenyl-3H-imidazo[4,5-b]pyridin-3-yl)benzyl)pyrrolidin-3-yl)amino)propan-1-ol

A mixture of benzyl (1-(4-(2-(2-aminopyridin-3-yl)-5-phenyl-3H-imidazo[4,5-b]pyridin-3-yl)benzyl)pyrrolidin-3-yl)(3-hydroxypropyl)carbamate (230 mg, 292 gmol) and Pd(OH)₂ (30 mg, 20% purity) in MeOH (2 mL) was degassed and purged with H₂ three times. The mixture was stirred at 40° C. for 6 hr under H₂ (30 Psi) atmosphere. The mixture was filtered and concentrated under reduced pressure to give 3-((1-(4-(2-(2-aminopyridin-3-yl)-5-phenyl-3H-imidazo[4,5-b]pyridin-3-yl)benzyl)pyrrolidin-3-yl)amino)propan-1-ol (108 mg) as a yellow oil. MS: m/z=520.2 [M+H]⁺.

### Step 6: 6-(1-(4-(2-(2-Aminopyridin-3-yl)-5-phenyl-3H-imidazo[4,5-b]pyridin-3-yl)benzyl)pyrrolidin-3-yl)-2-oxa-6-azabicyclo[5.2.0]non-1(7)-ene-8,9-dione

To a solution of 3-((1-(4-(2-(2-aminopyridin-3-yl)-5-phenyl-3H-imidazo[4,5-b]pyridin-3-yl)benzyl)pyrrolidin-3-yl)amino)propan-1-ol (100 mg, 129 μmol) and TEA (26.1 mg, 258 μmol) in MeOH (5 mL) was added 3,4-dimethoxycyclobut-3-ene-1,2-dione (23.8 mg, 168 μmol). The mixture was stirred at 25° C. for 12 hr. The mixture was concentrated under reduced pressure. After purified by prep-HPLC (column: [water (NH₄HCO₃)-ACN]; B %: 35%-65%, 18 min), 6-(1-(4-(2-(2-aminopyridin-3-yl)-5-phenyl-3H-imidazo[4,5-b]pyridin-3-yl)benzyl)pyrrolidin-3-yl)-2-oxa-6-azabicyclo[5.2.0]non-1(7)-ene-8,9-dione (Example 82, 16 mg, yield: 10%) was obtained as a light-yellow solid. MS: m/z=598.2 [M+H]⁺. ¹H NMR (400 MHz, Methanol-d₄) δ 8.20 (d, J=8.4 Hz, 1H), 8.06-8.01 (m, 2H), 7.98 (dd, J=5.2, 1.6 Hz, 1H), 7.94 (d, J=8.8 Hz, 1H), 7.57-7.53 (m, 2H), 7.47-7.32 (m, 6H), 6.48 (dd, J=7.6, 5.2 Hz, 1H), 5.44-5.32 (m, 1H), 4.52-4.42 (m, 2H), 3.80-3.67 (m, 4H), 3.05-2.88 (m, 2H), 2.68-2.58 (m, 1H), 2.46-2.36 (m, 1H), 2.35-2.26 (m, 1H), 2.25-2.18 (m, 2H), 2.10-2.02 (m, 1H).

### Example 83: 3-((4-(4-(2-(2-Aminopyridin-3-yl)-5-phenyl-3H-imidazo[4,5-b]pyridin-3-yl)benzyl)piperazin-1-yl)amino)-4-methoxycyclobut-3-ene-1,2-dione

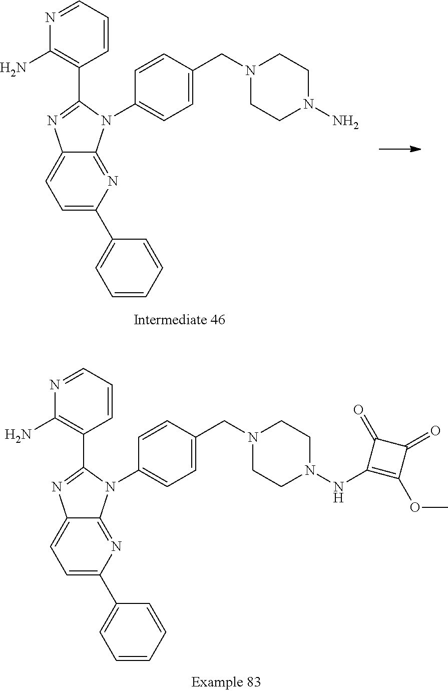 [SMILES: Nc1ncccc1-c1nc2ccc(-c3ccccc3)nc2n1-c1ccc(CN2CCN(N)CC2)cc1 ; COc1c(NN2CCN(Cc3ccc(-n4c(-c5cccnc5N)nc5ccc(-c6ccccc6)nc54)cc3)CC2)c(=O)c1=O]

To a solution of Intermediate 46 (100 mg, 209 μmol) and 3,4-dimethoxycyclobut-3-ene-1,2-dione (35.8 mg, 252 μmol) in MeOH (2 mL) was added TEA (106 mg, 1.05 mmol). The mixture was stirred at 25° C. for 16 hr. The reaction was filtered and concentrated under reduced pressure. 3-((4-(4-(2-(2-Aminopyridin-3-yl)-5-phenyl-3H-imidazo[4,5-b]pyridin-3-yl)benzyl)piperazin-1-yl)amino)-4-methoxycyclobut-3-ene-1,2-dione (Example 83, 52.4 mg, yield: 42%) was obtained as a yellow solid. MS: m/z=587.4 [M+H]⁺. ¹H NMR (400 MHz, Dimethylsulfoxide-d₆) δ 9.96 (br s, 1H), 8.27 (d, J=8.4 Hz, 1H), 8.05-7.97 (m, 4H), 7.49-7.43 (m, 6H), 7.42-7.37 (m, 1H), 7.15 (dd, J=8.0, 1.6 Hz, 1H), 7.01 (s, 2H), 6.38 (dd, J=7.6, 4.8 Hz, 1H), 4.31-4.25 (m, 3H), 3.59 (s, 2H), 3.27-3.20 (m, 3H), 3.17 (m, 1H), 2.90-2.83 (m, 4H).

### Example 84: 6-(1-(4-(2-(2-Aminopyridin-3-yl)-5-phenyl-3H-imidazo[4,5-b]pyridin-3-yl)benzyl)piperidin-4-yl)-2-oxa-6-azabicyclo[5.2.0]non-1(7)-ene-8,9-dione

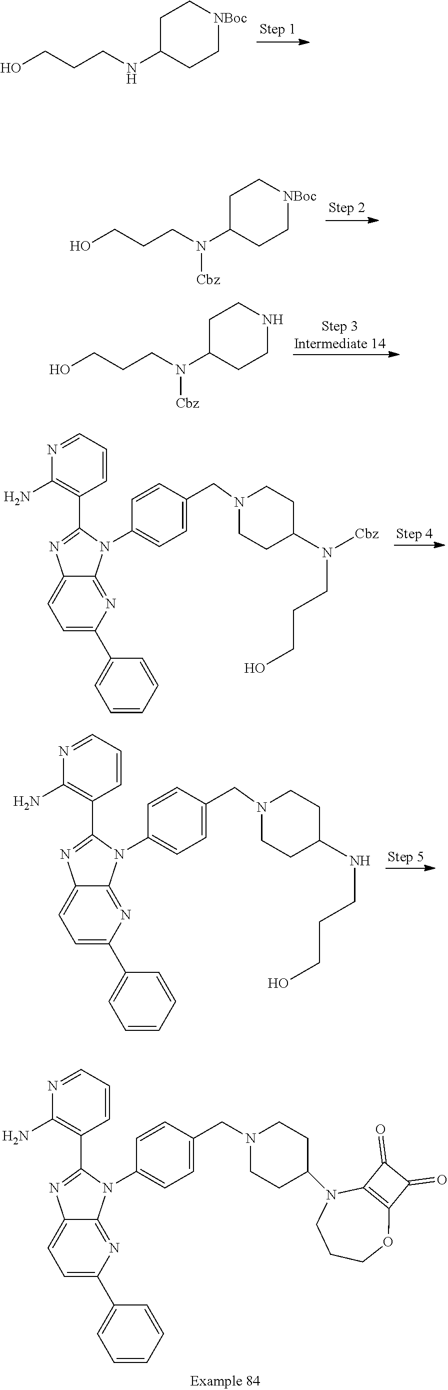

### Step 1: tert-Butyl 4-(((benzyloxy)carbonyl)(3-hydroxypropyl)amino)piperidine-1-carboxylate)

A mixture of tert-butyl 4-((3-hydroxypropyl)amino)piperidine-1-carboxylate (5 g, 19.3 mmol), TEA (3.92 g, 38.7 mmol) in CH₂Cl₂ (100 mL) was added CbzCl (3.63 g, 21.2 mmol). The mixture was stirred at 25° C. for 16 hr under N₂ atmosphere. The reaction mixture was quenched with H₂O (50 mL) at 10° C. and extracted with CH₂Cl₂ (100 mL×3). The combined organic layers were washed with brine (100 mL), dried over Na₂SO₄, filtered and concentrated under reduced pressure. tert-butyl 4-(((benzyloxy)carbonyl)(3-hydroxypropyl)amino)piperidine-1-carboxylate (6 g, yield: 80%) was obtained as a light-yellow oil. ¹H NMR (400 MHz, Chloroform-d) δ 7.38-7.31 (m, 5H), 5.17-5.12 (m, 2H), 4.26-4.13 (m, 2H), 4.05-3.76 (m, 1H), 3.62-3.52 (m, 2H), 3.46-3.19 (m, 2H), 2.77-2.63 (m, 2H), 1.71-1.63 (m, 6H), 1.44 (s, 9H).

### Step 2: Benzyl (3-hydroxypropyl)(piperidin-4-yl)carbamate)

A mixture of tert-butyl 4-(((benzyloxy)carbonyl)(3-hydroxypropyl)amino)piperidine-1-carboxylate (2 g, 5.10 mmol) in HCl/1,4-dioxane (4 M, 13.1 mL) was stirred at 25° C. for 0.5 hr. The reaction mixture was concentrated under reduced pressure to give benzyl (3-hydroxypropyl)(piperidin-4-yl)carbamate (1.5 g, HCl salt) as a light-yellow oil, which was used directly in the next step.

### Step 3: Benzyl (1-(4-(2-(2-aminopyridin-3-yl)-5-phenyl-3H-imidazo[4,5-b]pyridin-3-yl)benzyl)piperidin-4-yl)(3-hydroxypropyl)carbamate)

A mixture of Intermediate 14 (1 g, 2.43 mmol) and benzyl (3-hydroxypropyl)(piperidin-4-yl)carbamate (1.09 g, 2.67 mmol) in DMF (10 mL) were added K₂CO₃ (1.68 g, 12.1 mmol) and NaI (181 mg, 1.21 mmol). The mixture was stirred at 80° C. for 5 hr under N₂ atmosphere. The reaction mixture was diluted with H₂O (50 mL) and extracted with CH₂Cl₂ (50 mL×3). The combined organic layers were washed with brine (50 mL), dried over Na₂SO₄, filtered, and concentrated under reduced pressure. After purified by silica gel flash chromatography (MeOH in CH₂Cl₂=0% to 15%), benzyl (1-(4-(2-(2-aminopyridin-3-yl)-5-phenyl-3H-imidazo[4,5-b]pyridin-3-yl)benzyl)piperidin-4-yl)(3-hydroxypropyl)carbamate (250 mg, yield: 13%) was obtained as a yellow solid. MS: m/z=668.2 [M+H]⁺. ¹H NMR (400 MHz, Chloroform-d) δ 8.13 (d, J=8.4 Hz, 1H), 8.06 (dd, J=4.8, 1.2 Hz, 1H), 8.02 (d, J=7.2 Hz, 2H), 7.80 (d, J=8.4 Hz, 1H), 7.47-7.33 (m, 1OH), 7.09 (dd, J=8.0, 1.6 Hz, 1H), 6.59 (br s, 2H), 6.40-6.31 (m, 1H), 5.18 (s, 2H), 4.02-3.77 (m, 1H), 3.60 (s, 2H), 3.52-3.30 (m, 2H), 3.09-2.95 (m, 2H), 2.23-2.06 (m, 2H), 1.94-1.84 (m, 2H), 1.80-1.65 (m, 6H).

### Step 4: 3-((1-(4-(2-(2-Aminopyridin-3-yl)-5-phenyl-3H-imidazo[4,5-b]pyridin-3-yl)benzyl)piperidin-4-yl)amino)propan-1-ol)

A mixture of benzyl (1-(4-(2-(2-aminopyridin-3-yl)-5-phenyl-3H-imidazo[4,5-b]pyridin-3-yl)benzyl)piperidin-4-yl)(3-hydroxypropyl)carbamate (250 mg, 374 μmol), Pd(OH)₂/C (50 mg, 20% purity) in MeOH (5 mL) was degassed and purged with H₂ three times. The mixture was stirred at 40° C. for 6 hr under H₂ atmosphere (30 Psi). The reaction mixture was filtered and washed with CH₂Cl₂ (100 mL), concentrated under reduced pressure. After purified by prep-TLC (MeOH in CH₂Cl₂=10%), 3-((1-(4-(2-(2-aminopyridin-3-yl)-5-phenyl-3H-imidazo[4,5-b]pyridin-3-yl)benzyl)piperidin-4-yl)amino)propan-1-ol (75 mg, yield: 36%) was obtained as a light-yellow solid. MS: m/z=534.3 [M+H]⁺. ¹H NMR (400 MHz, Methanal-d₄) δ 8.19 (d, J=8.4 Hz, 1H), 8.05-8.01 (m, 2H), 7.97 (dd, J=5.2, 2.0 Hz, 1H), 7.94 (d, J=8.4 Hz, 1H), 7.52 (d, J=8.4 Hz, 2H), 7.46-7.37 (m, 5H), 7.31 (dd, J=7.6, 2.0 Hz, 1H), 6.46 (dd, J=7.6, 5.2 Hz, 1H), 3.70-3.65 (m, 2H), 3.64 (s, 2H), 3.04-2.96 (m, 4H), 2.93-2.85 (m, 1H), 2.20-2.10 (m, 2H), 2.30-1.80 (m, 2H), 1.85-1.79 (m, 2H), 1.64-1.55 (m, 2H).

### Step 5: 6-(1-(4-(2-(2-Aminopyridin-3-yl)-5-phenyl-3H-imidazo[4,5-b]pyridin-3-yl)benzyl)piperidin-4-yl)-2-oxa-6-azabicyclo[5.2.0]non-1(7)-ene-8,9-dione)

A mixture of 3-((1-(4-(2-(2-aminopyridin-3-yl)-5-phenyl-3H-imidazo[4,5-b]pyridin-3-yl)benzyl)piperidin-4-yl)amino)propan-1-ol (60 mg, 112 μmol), TEA (34.1 mg, 337 mol) and 3,4-dimethoxycyclobut-3-ene-1,2-dione (17.5 mg, 123 μmol) in MeOH (1 mL) was stirred at 25° C. for 16 hr. The reaction mixture was concentrated under reduced pressure. The residue was washed with MeOH (10 mL) and filtered. The filtrate was concentrated and purified by prep-TLC (MeOH in CH₂Cl₂=10%) to give 6-(1-(4-(2-(2-aminopyridin-3-yl)-5-phenyl-3H-imidazo[4,5-b]pyridin-3-yl)benzyl)piperidin-4-yl)-2-oxa-6-azabicyclo[5.2.0]non-1(7)-ene-8,9-dione (Example 84, 9.2 mg, yield: 12%) as a white solid. MS: m/z=612.3 [M+H]⁺. ¹H NMR (400 MHz, Acetonitrile-d₃) δ 8.17 (d, J=8.4 Hz, 1H), 8.02 (d, J=6.4 Hz, 2H), 7.90 (d, J=8.8 Hz, 1H), 7.52-7.39 (m, 8H), 7.15 (d, J=7.6 Hz, 1H), 6.58 (br s, 2H), 6.38 (dd, J=7.6, 5.2 Hz, 1H), 4.45-4.40 (m, 3H), 3.62 (s, 2H), 3.52-3.48 (m, 2H), 3.05-2.95 (m, 2H), 1.80-1.70 (m, 2H), 1.30-1.20 (m, 4H), 0.90-0.83 (m, 2H).

### Example 85: 6-(1-(4-(2-(2-Aminopyridin-3-yl)-5-phenyl-3H-imidazo[4,5-b]pyridin-3-yl)benzyl)piperidin-4-yl)-2-thia-6-azabicyclo[5.2.0]non-1(7)-ene-8,9-dione

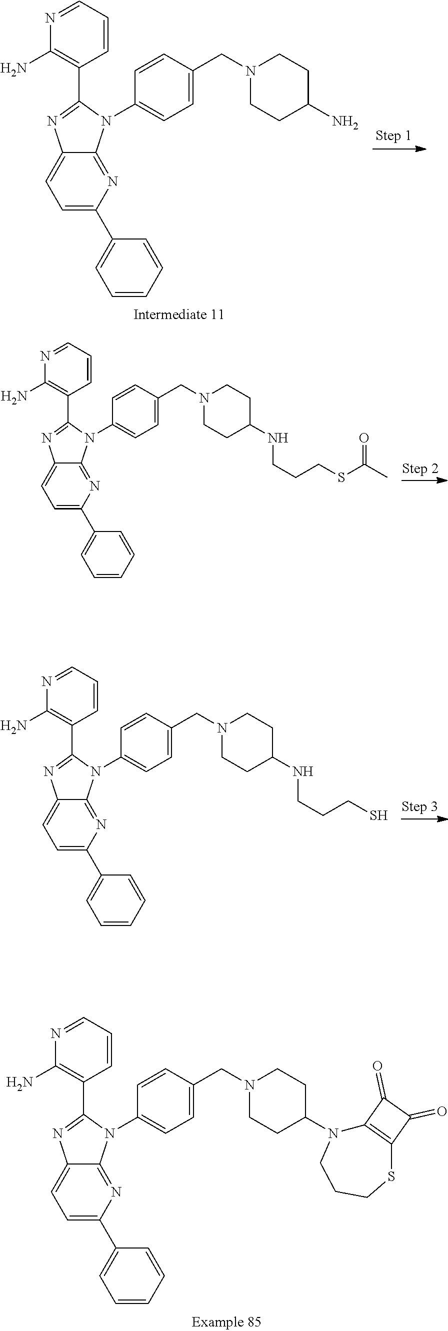 [SMILES: Nc1ncccc1-c1nc2ccc(-c3ccccc3)nc2n1-c1ccc(CN2CCC(N)CC2)cc1 ; CC(=O)SCCCNC1CCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCC(NCCCS)CC2)cc1 ; Nc1ncccc1-c1nc2ccc(-c3ccccc3)nc2n1-c1ccc(CN2CCC(N3CCCSc4c3c(=O)c4=O)CC2)cc1]

### Step 1: S-(3-((1-(4-(2-(2-Aminopyridin-3-yl)-5-phenyl-3H-imidazo[4,5-b]pyridin-3-yl)benzyl)piperidin-4-yl)amino)propyl)ethanethioate

To a solution of Intermediate 11 (500 mg, 1.05 mmol) and S-(3-bromopropyl)ethanethioate (186 mg, 946 μmol) in CH₃CN (10 mL) were added K₂CO₃ (581 mg, 4.21 mmol) and NaI (31.5 mg, 210 μmol). The mixture was stirred at 80° C. for 5 hr. The reaction mixture was filtered and concentrated under reduced pressure. After purified by silica gel flash chromatography (MeOH in CH₂Cl₂=5% to 7%), S-(3-((1-(4-(2-(2-aminopyridin-3-yl)-5-phenyl-3H-imidazo[4,5-b]pyridin-3-yl)benzyl)piperidin-4-yl)amino)propyl)ethanethioate (120 mg, yield: 16%) was obtained as a yellow solid. MS: m/z=592.3 [M+H]⁺.

### Step 2: 3-((1-(4-(2-(2-aminopyridin-3-yl)-5-phenyl-3H-imidazo[4,5-b]pyridin-3-yl)benzyl)piperidin-4-yl)amino)propane-1-thiol

To a solution of S-(3-((1-(4-(2-(2-aminopyridin-3-yl)-5-phenyl-3H-imidazo[4,5-b]pyridin-3-yl)benzyl)piperidin-4-yl)amino)propyl)ethanethioate (120 mg, 202 μmol) in MeOH (5 mL) was added HCl (12 M, 50.7 μL). The mixture was stirred at 60° C. for 16 hr. The reaction mixture was quenched with saturated Na₂CO₃ (5 mL), diluted with H₂O (5 mL), and extracted with CH₂Cl₂ (20 mL×3). The combined organic layers were washed with brine (20 mL), dried over Na₂SO₄, filtered and concentrated under reduced pressure to give 3-((1-(4-(2-(2-aminopyridin-3-yl)-5-phenyl-3H-imidazo[4,5-b]pyridin-3-yl)benzyl)piperidin-4-yl)amino)propane-1-thiol (100 mg crude) as a yellow solid, which was used directly in the next step. MS: m/z=550.3 [M+H]r.

### Step 3: 6-(1-(4-(2-(2-aminopyridin-3-yl)-5-phenyl-3H-imidazo[4,5-b]pyridin-3-yl)benzyl)piperidin-4-yl)-2-thia-6-azabicyclo[5.2.0]non-1(7)-ene-8,9-dione

To a solution of 3-((1-(4-(2-(2-aminopyridin-3-yl)-5-phenyl-3H-imidazo[4,5-b]pyridin-3-yl)benzyl)piperidin-4-yl)amino)propane-1-thiol (100 mg, 181 μmol) and 3,4-dimethoxycyclobut-3-ene-1,2-dione (25.8 mg, 181 gmol) in MeOH (1 mL) was added TEA (55.2 mg, 545 μmol). The mixture was stirred at 20° C. for 16 hr. The reaction mixture was concentrated under reduced pressure. After purified by silica gel flash chromatography (MeOH in CH₂Cl₂=0% to 7%), and then purified by prep-HPLC (column: Waters xbridge 150×25 mm 10 um; mobile phase: [water (NH₄HCO₃)-ACN]; gradient: 39%-69% B over 10 min), 6-(1-(4-(2-(2-aminopyridin-3-yl)-5-phenyl-3H-imidazo[4,5-b]pyridin-3-yl)benzyl)piperidin-4-yl)-2-thia-6-azabicyclo[5.2.0]non-1(7)-ene-8,9-dione (Example 85, 4.2 mg, yield: 3.3%) was obtained as a light-yellow solid. MS: m/z=628.5 [M+H]⁺. ¹H NMR (400 MHz, Methanol-d₄) δ 8.20 (d, J=8.4 Hz, 1H), 8.05-8.02 (m, 2H), 7.99 (dd, J=5.2, 2.0 Hz, 1H), 7.95 (d, J=8.4 Hz, 1H), 7.54 (d, J=8.4 Hz, 2H), 7.48-7.39 (m, 5H), 7.32 (dd, J=7.6, 1.6 Hz, 1H), 6.50 (dd, J=7.6, 5.2 Hz, 1H), 3.68 (s, 2H), 3.63-3.60 (m, 2H), 3.50-3.47 (m, 1H), 3.15-3.12 (m, 2H), 3.10-3.05 (m, 2H), 2.36-2.31 (m, 2H), 2.25-2.20 (m, 2H), 1.89-1.82 (m, 4H).

### Example 86: 3-((3aR,6aS)-5-(4-(2-(2-Aminopyridin-3-yl)-5-phenyl-3H-imidazo[4,5-b]pyridin-3-yl)benzyl)hexahydropyrrolo[3,4-c]pyrrol-2(1H)-yl)-4-methoxycyclobut-3-ene-1,2-dione

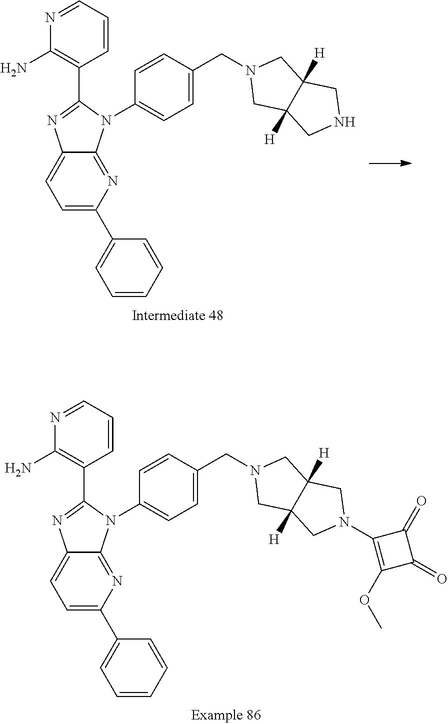 [SMILES: Nc1ncccc1-c1nc2ccc(-c3ccccc3)nc2n1-c1ccc(CN2C[C@H]3CNC[C@H]3C2)cc1 ; COc1c(N2C[C@H]3CN(Cc4ccc(-n5c(-c6cccnc6N)nc6ccc(-c7ccccc7)nc65)cc4)C[C@H]3C2)c(=O)c1=O]

To a mixture of Intermediate 48 (200 mg, 382 μmol, HCl) and TEA (116 mg, 1.14 mmol) in MeOH (1.5 mL) was added 3,4-dimethoxycyclobut-3-ene-1,2-dione (60.0 mg, 420 gmol) at 0° C., the mixture was stirred at 25° C. for 16 hr under N₂. The reaction mixture was concentrated and purified by silica gel flash chromatography (MeOH in DCM=0% to 6%) to give 3-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-4-methoxycyclobut-3-ene-1,2-dione (Example 86, 160 mg, yield 70%) as a yellow solid. MS: m/z=620.1 [M+Na]⁺. ¹H NMR (400 MHz, Dimethylsulfoxide-d₆) δ 8.27 (d, J=8.4 Hz, 1H), 8.05-7.97 (m, 4H), 7.49-7.36 (m, 7H), 7.16 (dd, J=8.0, 2.0 Hz, 1H), 7.02 (s, 2H), 6.39 (dd, J=7.6, 4.8 Hz, 1H), 4.26 (s, 3H), 3.92-3.80 (m, 1H), 3.76-3.59 (m, 4H), 3.44-3.38 (m, 3H), 2.91-2.82 (m, 2H), 2.48-2.42 (m, 2H).

### Example 87: 3-((3aR,6aS)-5-(4-(2-(2-Aminopyridin-3-yl)-5-phenyl-3H-imidazo[4,5-b]pyridin-3-yl)benzyl)hexahydropyrrolo[3,4-c]pyrrol-2(1H)-yl)-4-(methylthio)cyclobut-3-ene-1,2-dione

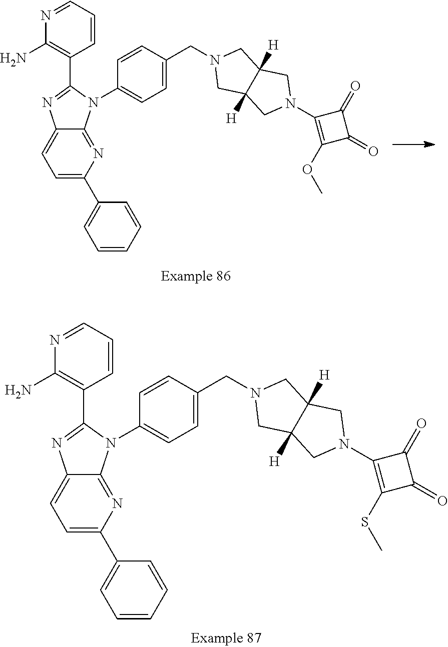 [SMILES: COc1c(N2C[C@H]3CN(Cc4ccc(-n5c(-c6cccnc6N)nc6ccc(-c7ccccc7)nc65)cc4)C[C@H]3C2)c(=O)c1=O ; CSc1c(N2C[C@H]3CN(Cc4ccc(-n5c(-c6cccnc6N)nc6ccc(-c7ccccc7)nc65)cc4)C[C@H]3C2)c(=O)c1=O]

To a solution of Example 86 (60 mg, 100 gmol) in EtOH (2 mL) was added NaSH (33.8 mg, 602 μmol) at 20° C. The reaction mixture was stirred at 20° C. for 12 hr. Then CH₃I (21.4 mg, 151 μmol) was added at 20° C. The reaction mixture was stirred at 20° C. for 2 hr and concentrated under reduced pressure at 30° C. The residue was purified by prep-HPLC (column: Welch Xtimate C18 150×30 mm×5 μm; mobile phase: [water (NH₃H₂O+NH₄HCO₃)-ACN]; gradient: 60%-90% B over 7 min) to give 3-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-4-(methylthio)cyclobut-3-ene-1,2-dione (Example 87, 6.7 mg, yield:11%) as a gray solid. MS: m/z=614.2 [M+H]⁺. ¹H NMR (400 MHz, Dimethylsulfoxide-d₆) 8.27 (d, J=8.8 Hz, 1H), 8.06-7.96 (m, 4H), 7.49-7.37 (m, 7H), 7.18-7.13 (m, 1H), 7.02 (s, 2H), 6.39 (dd, J=8.0, 4.8 Hz, 1H), 3.96-3.83 (m, 2H), 3.78-3.72 (m, 1H), 3.70-3.57 (m, 2H), 3.54-3.48 (m, 1H), 3.40-3.35 (m, 2H), 3.33-3.30 (m, 2H), 2.92-2.85 (m, 2H), 2.80 (s, 3H).

### Example 88: 3-((3aR,6aS)-5-(4-(2-(2-Aminopyridin-3-yl)-5-phenyl-3H-imidazo[4,5-b]pyridin-3-yl)benzyl)hexahydropyrrolo[3,4-c]pyrrol-2(1H)-yl)-4-(ethylthio)cyclobut-3-ene-1,2-dione

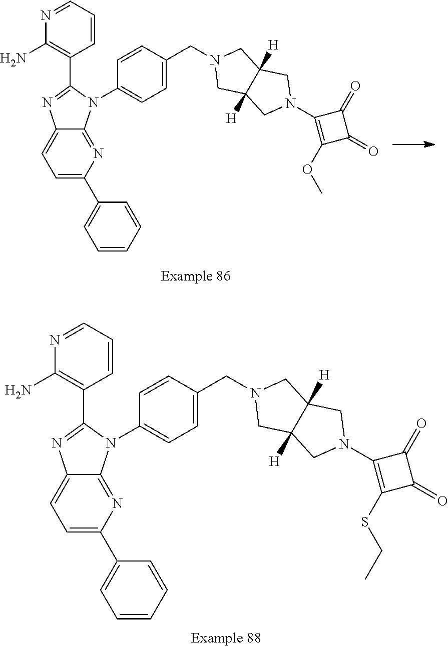 [SMILES: COc1c(N2C[C@H]3CN(Cc4ccc(-n5c(-c6cccnc6N)nc6ccc(-c7ccccc7)nc65)cc4)C[C@H]3C2)c(=O)c1=O ; CCSc1c(N2C[C@H]3CN(Cc4ccc(-n5c(-c6cccnc6N)nc6ccc(-c7ccccc7)nc65)cc4)C[C@H]3C2)c(=O)c1=O]

To a solution of Example 86 (150 mg, 251 gmol) in CH₂Cl₂ (5 mL) were added TEA (127 mg, 1.25 mmol) and EtSH (1.97 g, 31.7 mmol). The mixture was stirred at 20° C. for 12 hr. The reaction mixture was quenched with H₂O (10 mL) at 0° C. and extracted with CH₂Cl₂ (15 mL×2). The combined organic layers were washed with brine (15 mL), dried over anhydrous Na₂SO₄, filtered, and concentrated under reduced pressure. After purified by prep-HPLC (column: Phenomenex C18 80×40 mm×3 μm; mobile phase: [water (NH₄HCO₃)-ACN]; gradient: 60%-90% B over 8 min), 3-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-4-(ethylthio)cyclobut-3-ene-1,2-dione (Example 88, 20.7 mg, yield: 13%) was obtained as a yellow solid. MS: m/z=628.3 [M+H]⁺. ¹H NMR (400 MHz, Dimethylsulfoxide-d₆) 8.27 (d, J=8.4 Hz, 1H), 8.05-7.97 (m, 4H), 7.49-7.37 (m, 7H), 7.15 (dd, J=7.6, 2.0 Hz, 1H), 7.03 (s, 2H), 6.39 (dd, J=7.6, 4.8 Hz, 1H), 3.97-3.82 (m, 2H), 3.78-3.72 (m, 1H), 3.70-3.57 (m, 2H), 3.54-3.48 (m, 1H), 3.42-3.35 (m, 2H), 3.33-3.28 (m, 1H), 2.89 (br s, 2H), 2.57-2.52 (m, 3H), 1.32 (t, J=7.6 Hz, 3H).

### Example 89: (S)-7-(4-(2-(2-Aminopyridin-3-yl)-5-phenyl-3H-imidazo[4,5-b]pyridin-3-yl)benzyl)-5,5a,6,7,8,9-hexahydro-4H-cyclobuta[b]pyrazino[1,2-d][1,4]oxazepine-1,2-dione

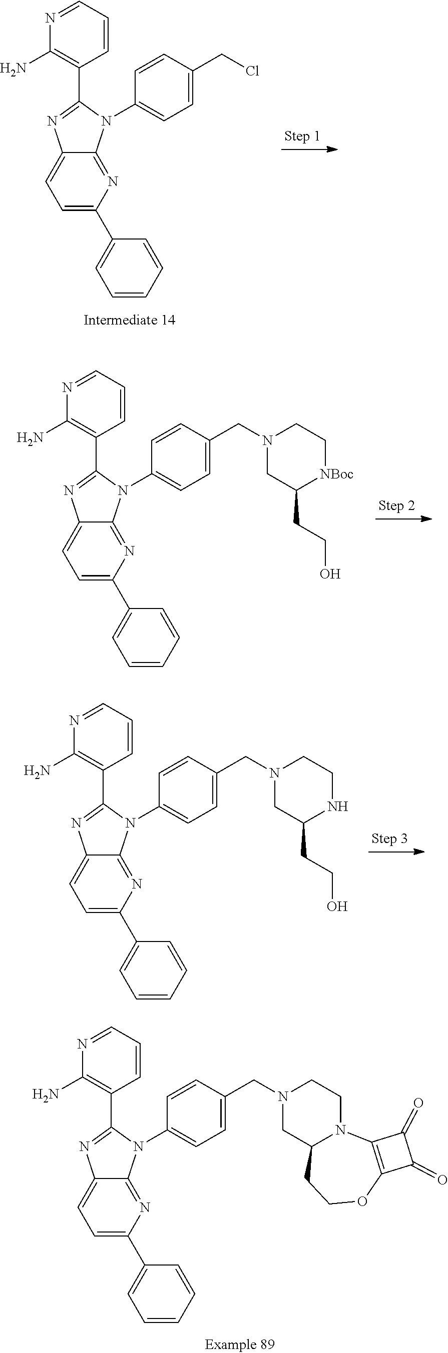

### Step 1: tert-Butyl (1-(4-((6-morpholino-3-nitropyridin-2-yl)amino)benzyl)piperidin-4-yl)carbamate

To a solution of Intermediate 14 (200 mg, 485 gmol) in DMF (3 mL) were added tert-butyl (S)-2-(2-hydroxyethyl)piperazine-1-carboxylate (123 mg, 534 gmol), NaI (14.5 mg, 97.1 gmol) and K₂CO₃ (268 mg, 1.94 mmol). The mixture was stirred at 80° C. for 1.5 hr. The reaction mixture was diluted with H₂O (20 mL) and filtered. The filter cake was concentrated under reduced pressure. After purified by silica gel flash chromatography (MeOH in CH₂Cl₂=0% to 3%), tert-butyl (S)-4-(4-(2-(2-aminopyridin-3-yl)-5-phenyl-3H-imidazo[4,5-b]pyridin-3-yl)benzyl)-2-(2-hydroxyethyl)piperazine-1-carboxylate (130 mg, yield: 34%) was obtained as a yellow solid. MS: m/z=606.4 [M+H]⁺. ¹H NMR (400 MHz, Chloroform-d) δ 8.13 (d, J=8.4 Hz, 1H), 8.06 (dd, J=4.8, 1.2 Hz, 1H), 8.01 (d, J=7.2 Hz, 2H), 7.81 (d, J=8.4 Hz, 1H), 7.50-7.37 (m, 7H), 7.12 (dd, J=7.6, 1.2 Hz, 1H), 6.73 (br s, 2H), 6.38 (dd, J=7.6, 4.8 Hz, 1H), 3.93-3.84 (m, 1H), 3.68-3.56 (m, 3H), 3.47-3.33 (m, 1H), 3.12-3.04 (m, 1H), 2.87-2.79 (m, 1H), 2.80-2.70 (m, 1H), 2.37-2.22 (m, 2H), 2.16-2.10 (m, 1H), 1.93-1.79 (m, 2H), 1.48 (s, 9H).

### Step 2: (S)-2-(4-(4-(2-(2-Aminopyridin-3-yl)-5-phenyl-3H-imidazo[4,5-b]pyridin-3-yl)benzyl)piperazin-2-yl)ethan-1-ol

To a solution of tert-butyl (S)-4-(4-(2-(2-aminopyridin-3-yl)-5-phenyl-3H-imidazo[4,5-b]pyridin-3-yl)benzyl)-2-(2-hydroxyethyl)piperazine-1-carboxylate (130 mg, 214 gmol,) in CH₂Cl₂ (2 mL) was added HCl/1,4-dioxane (4 M, 0.5 mL). The mixture was stirred at 20° C. for 0.2 hr. The reaction mixture was concentrated under reduced pressure to give (S)-2-(4-(4-(2-(2-aminopyridin-3-yl)-5-phenyl-3H-imidazo[4,5-b]pyridin-3-yl)benzyl)piperazin-2-yl)ethan-1-ol (120 mg, HCl salt) as a yellow solid, which was used directly in the next step. MS: m/z=506.3 [M+H]⁺.

### Step 3: (S)-7-(4-(2-(2-Aminopyridin-3-yl)-5-phenyl-3H-imidazo[4,5-b]pyridin-3-yl)benzyl)-5,5a,6,7,8,9-hexahydro-4H-cyclobuta[b]pyrazino[1,2-d][1,4]oxazepine-1,2-dione

To a solution of (S)-2-(4-(4-(2-(2-aminopyridin-3-yl)-5-phenyl-3H-imidazo[4,5-b]pyridin-3-yl)benzyl)piperazin-2-yl)ethan-1-ol (100 mg, 161 gmol) in MeOH (3 mL) were added 3,4-dimethoxycyclobut-3-ene-1,2-dione (22.9 mg, 161 μmol) and TEA (81.6 mg, 806 gmol). The mixture was stirred at 60° C. for 16 hr. The reaction mixture was concentrated under reduced pressure. After purified by prep-HPLC (column: Waters xbridge 150×25 mm 10 um; mobile phase: [water (NH₄HCO₃)-ACN]; gradient: 35%-65% B over 10 min), (S)-7-(4-(2-(2-aminopyridin-3-yl)-5-phenyl-3H-imidazo[4,5-b]pyridin-3-yl)benzyl)-5,5a,6,7,8,9-hexahydro-4H-cyclobuta[b]pyrazino[1,2-d][1,4]oxazepine-1,2-dione (Example 89, 15.8 mg, yield: 16%) was obtained as a light-yellow powder. MS: m/z=584.3 [M+H]⁺. ¹H NMR (400 MHz, Dimethylsulfoxide-d₆) δ 8.27 (d, J=8.4 Hz, 1H), 8.04-7.97 (m, 4H), 7.52-7.45 (m, 6H), 7.42-7.38 (m, 1H), 7.16 (dd, J=7.6, 1.6 Hz, 1H), 7.01 (br s, 2H), 6.40 (dd, J=7.6, 4.4 Hz, 1H), 4.64-4.56 (m, 1H), 4.50-4.42 (m, 1H), 4.38-4.31 (m, 1H), 3.69-3.61 (m, 2H), 3.58-3.50 (m, 1H), 3.28-3.23 (m, 1H), 2.93-2.82 (m, 2H), 2.26-2.19 (m, 1H), 2.14-1.97 (m, 3H).

### Example 90: 3-((1-(1-(4-(2-(2-Aminopyridin-3-yl)-5-phenyl-3H-imidazo[4,5-b]pyridin-3-yl)phenyl)ethyl)piperidin-4-yl)(methyl)amino)-4-methoxycyclobut-3-ene-1,2-dione

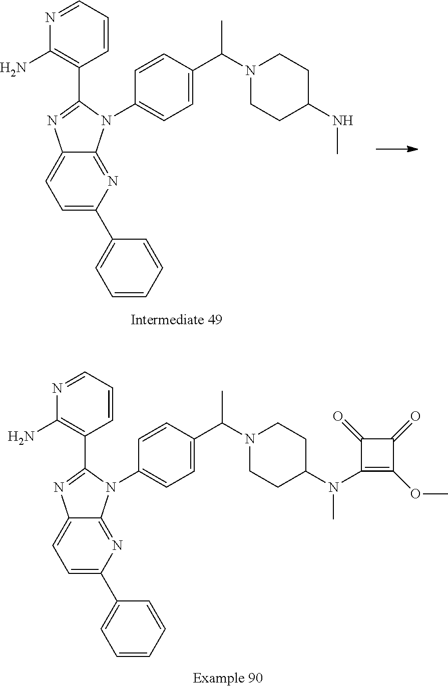 [SMILES: CNC1CCN(C(C)c2ccc(-n3c(-c4cccnc4N)nc4ccc(-c5ccccc5)nc43)cc2)CC1 ; COc1c(N(C)C2CCN(C(C)c3ccc(-n4c(-c5cccnc5N)nc5ccc(-c6ccccc6)nc54)cc3)CC2)c(=O)c1=O]

To a solution of Intermediate 49 (80 mg, 159 gmol) and 3,4-dimethoxycyclobut-3-ene-1,2-dione (27.1 mg, 191 μmol) in MeOH (2 mL) was added TEA (80.4 mg, 794 mol). The mixture was stirred at 25° C. for 16 hr. The reaction mixture was filtrated. The solid was collected, washed with MeOH (10 mL×2), and dried under reduced pressure. 3-((1-(1-(4-(2-(2-Aminopyridin-3-yl)-5-phenyl-3H-imidazo[4,5-b]pyridin-3-yl)phenyl)ethyl)piperidin-4-yl)(methyl)amino)-4-methoxycyclobut-3-ene-1,2-dione (Example 90, 33.3 mg, yield: 34%) was obtained as a yellow solid. MS: m/z=614.3. [M+H]⁺. ¹H NMR (400 MHz, Dimethylsulfoxide-d₆) δ 8.27 (d, J=8.4 Hz, 1H), 8.05-7.96 (m, 4H), 7.52-7.39 (m, 7H), 7.15-7.01 (m, 3H), 6.44-6.27 (m, 1H), 4.33-4.24 (m, 3H), 4.19-4.11 (m, 0.5H), 3.76-3.65 (m, 1H), 3.59-3.51 (m, 0.5H), 3.19 (s, 1H), 3.02 (s, 3H), 2.93-2.85 (m, 1H), 2.00-1.64 (m, 6H), 1.42-1.32 (m, 3H).

### Example 91: 3-((1-(4-(2-(2-Aminopyridin-3-yl)-5-phenyl-3H-imidazo[4,5-b]pyridin-3-yl)benzyl)piperidin-4-yl)(ethyl)amino)-4-methoxycyclobut-3-ene-1,2-dione

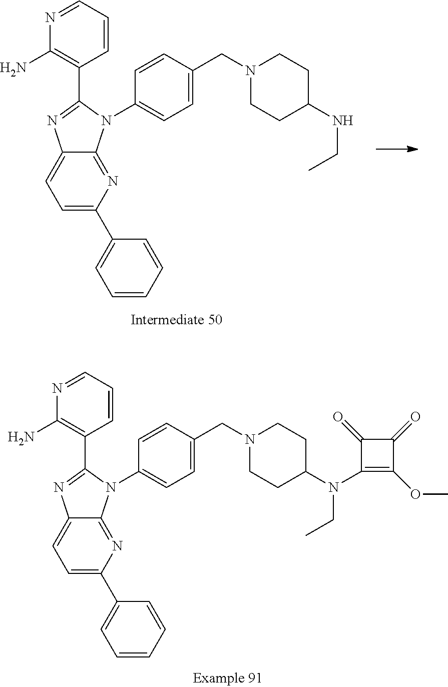 [SMILES: CCNC1CCN(Cc2ccc(-n3c(-c4cccnc4N)nc4ccc(-c5ccccc5)nc43)cc2)CC1 ; CCN(c1c(OC)c(=O)c1=O)C1CCN(Cc2ccc(-n3c(-c4cccnc4N)nc4ccc(-c5ccccc5)nc43)cc2)CC1]

To a solution of Intermediate 50 (50 mg, 99.3 mol) and 3,4-dimethoxycyclobut-3-ene-1,2-dione (16.9 mg, 119 μmol) in MeOH (2 mL) was added TEA (50.2 mg, 496 mol). The resulting mixture was stirred at 25° C. for 16 hr. The reaction mixture was filtrated. The solid was collected, washed with MeOH (10 mL×2), dried under reduced pressure. After triturated with MeOH (20 mL), 3-((1-(4-(2-(2-aminopyridin-3-yl)-5-phenyl-3H-imidazo[4,5-b]pyridin-3-yl)benzyl)piperidin-4-yl)(ethyl)amino)-4-methoxycyclobut-3-ene-1,2-dione (Example 91, 16.5 mg, yield: 26%) was obtained as a yellow solid. MS: m/z=614.4. [M+H]⁺. ¹H NMR (400 MHz, Dimethylsulfoxide-d₆) δ 8.27 (d, J=8.4 Hz, 1H), 8.06-7.97 (m, 4H), 7.49-7.43 (m, 6H), 7.42-7.38 (m, 1H), 7.17-7.12 (m, 1H), 7.02 (br s, 2H), 6.42-6.35 (m, 1H), 4.33-4.29 (m, 3H), 4.20-4.14 (m, 0.5H), 3.65-3.63 (m, 0.5H), 3.61-3.58 (m, 2H), 3.45-3.34 (m, 2H), 2.97-2.91 (m, 2H), 2.09-2.00 (m, 2H), 1.93-1.82 (m, 2H), 1.77-1.69 (m, 2H), 1.16 (t, J=6.8 Hz, 3H).

### Example 92: 3-((1-(4-(2-(2-Aminopyridin-3-yl)-5-phenyl-3H-imidazo[4,5-b]pyridin-3-yl)benzyl)piperidin-4-yl)(ethyl)amino)-4-(methylthio)cyclobut-3-ene-1,2-dione

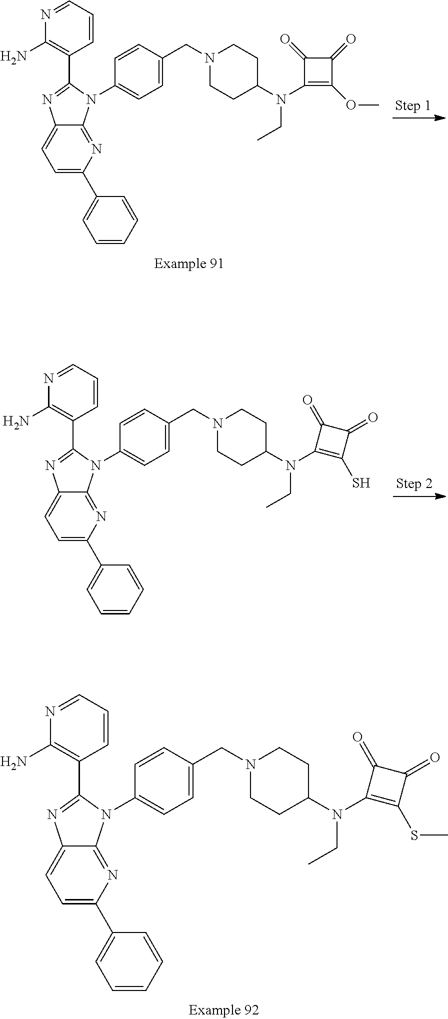 [SMILES: CCN(c1c(OC)c(=O)c1=O)C1CCN(Cc2ccc(-n3c(-c4cccnc4N)nc4ccc(-c5ccccc5)nc43)cc2)CC1 ; CCN(c1c(S)c(=O)c1=O)C1CCN(Cc2ccc(-n3c(-c4cccnc4N)nc4ccc(-c5ccccc5)nc43)cc2)CC1 ; CCN(c1c(SC)c(=O)c1=O)C1CCN(Cc2ccc(-n3c(-c4cccnc4N)nc4ccc(-c5ccccc5)nc43)cc2)CC1]

### Step 1: 3-((1-(4-(2-(2-Aminopyridin-3-yl)-5-phenyl-3H-imidazo[4,5-b]pyridin-3-yl)benzyl)piperidin-4-yl)(ethyl)amino)-4-mercaptocyclobut-3-ene-1,2-dione

To a solution of Example 91 (100 mg, 163 mol) in EtOH (5 mL) was added NaHS (45.7 mg, 815 μmol). The mixture was stirred at 25° C. for 24 hr. The reaction was concentrated under reduced pressure to give 3-((1-(4-(2-(2-aminopyridin-3-yl)-5-phenyl-3H-imidazo[4,5-b]pyridin-3-yl)benzyl)piperidin-4-yl)(ethyl)amino)-4-mercaptocyclobut-3-ene-1,2-dione (100 mg, crude) as a yellow solid. MS: m/z=616.3. [M+H]⁺.

### Step 2: 3-((1-(4-(2-(2-Aminopyridin-3-yl)-5-phenyl-3H-imidazo[4,5-b]pyridin-3-yl)benzyl)piperidin-4-yl)(ethyl)amino)-4-(methylthio)cyclobut-3-ene-1,2-dione

To a solution of 3-((1-(4-(2-(2-aminopyridin-3-yl)-5-phenyl-3H-imidazo[4,5-b]pyridin-3-yl)benzyl)piperidin-4-yl)(ethyl)amino)-4-mercaptocyclobut-3-ene-1,2-dione (100 mg, 163 μmol) in EtOH (5 mL) was added CH₃I (69.4 mg, 489 mol). The mixture was stirred at 25° C. for 16 hr. The reaction was concentrated under reduced pressure. After purified by prep-HPLC (column: Waters xbridge 150×25 mm×10 m; mobile phase: [water (NH₄HCO₃)-ACN]; gradient: 49%-79%, over 4 min), 3-((1-(4-(2-(2-aminopyridin-3-yl)-5-phenyl-3H-imidazo[4,5-b]pyridin-3-yl)benzyl)piperidin-4-yl)(ethyl)amino)-4-(methylthio)cyclobut-3-ene-1,2-dione (Example 92, 6.5 mg, yield: 6.2%) was obtained as a yellow solid. MS: m/z=630.4. [M+H]⁺. ¹H NMR (400 MHz, Dimethylsulfoxide-d₆) δ 8.28 (d, J=8.4 Hz, 1H), 8.05-7.97 (m, 4H), 7.51-7.44 (m, 6H), 7.43-7.39 (m, 1H), 7.18-7.12 (m, 1H), 7.03 (br s, 2H), 6.43-6.34 (m, 1H), 4.40-4.30 (m, 0.5H), 3.67-3.65 (m, 0.5H), 3.60 (s, 2H), 3.51-3.35 (m, 2H), 2.99-2.93 (m, 2H), 2.89-2.85 (m, 3H), 2.08-2.02 (m, 2H), 1.91-1.74 (m, 4H), 1.25-1.19 (m, 3H).

### Example 93: 3-((1-(4-(2-(2-Aminopyridin-3-yl)-5-phenyl-3H-imidazo[4,5-b]pyridin-3-yl)benzyl)piperidin-4-yl)(ethyl)amino)-4-(ethylthio)cyclobut-3-ene-1,2-dione

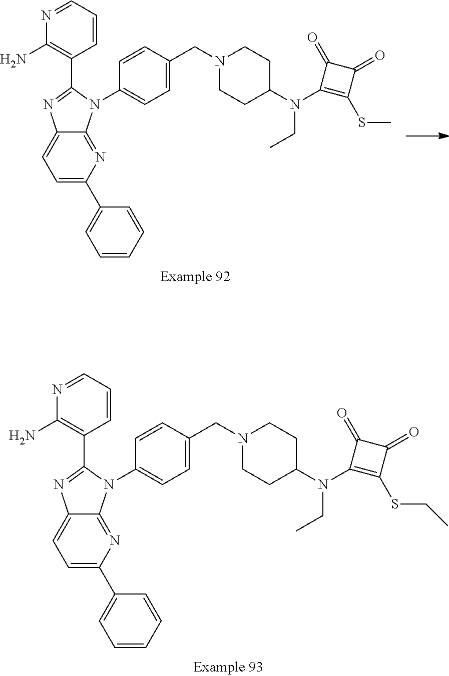 [SMILES: CCN(c1c(SC)c(=O)c1=O)C1CCN(Cc2ccc(-n3c(-c4cccnc4N)nc4ccc(-c5ccccc5)nc43)cc2)CC1 ; CCSc1c(N(CC)C2CCN(Cc3ccc(-n4c(-c5cccnc5N)nc5ccc(-c6ccccc6)nc54)cc3)CC2)c(=O)c1=O]

To a solution of Example 92 (100 mg, 163 gmol) in CH₂Cl₂ (2 mL) were added TEA (82.4 mg, 815 μmol) and ethanethiol (1.19 g, 19.2 mmol). The mixture was stirred at 25° C. for 24 hr. The reaction mixture was quenched with H₂O (5 mL) at 0° C., diluted with H₂O (10 mL) and extracted with CH₂Cl₂ (15 mL×2). The combined organic layers were washed with brine (15 mL), dried over Na₂SO₄, filtered, and concentrated under reduced pressure. After purified by prep-HPLC (column: Waters xbridge 150×25 mm×10 μm; mobile phase: [water (NH₄HCO₃)-ACN]; gradient: 44%-74%, over 14 min), 3-((1-(4-(2-(2-aminopyridin-3-yl)-5-phenyl-3H-imidazo[4,5-b]pyridin-3-yl)benzyl)piperidin-4-yl)(ethyl)amino)-4-(ethylthio)cyclobut-3-ene-1,2-dione (Example 93, 5.6 mg, yield: 5.2%) was obtained as a yellow solid. MS: m/z=644.5. [M+H]⁺. ¹H NMR (400 MHz, Dimethylsulfoxide-d₆) δ 8.27 (d, J=8.0 Hz, 1H), 8.05-7.97 (m, 4H), 7.50-7.43 (m, 6H), 7.42-7.38 (m, 1H), 7.17-7.12 (m, 1H), 7.02 (br s, 2H), 6.44-6.31 (m, 1H), 4.35-4.29 (m, 0.5H), 3.68-3.66 (m, 0.5H), 3.60 (s, 2H), 3.45-3.41 (m, 2H), 2.97-2.91 (m, 2H), 2.52-2.51 (m, 2H), 2.07-2.01 (m, 2H), 1.90-1.72 (m, 4H), 1.36-1.30 (m, 3H), 1.24-1.18 (m, 3H).

### Example 94: 3-((1-(4-(2-(2-Aminopyridin-3-yl)-5-phenyl-3H-imidazo[4,5-b]pyridin-3-yl)benzyl)piperidin-4-yl)(cyclopropyl)amino)-4-methoxycyclobut-3-ene-1,2-dione

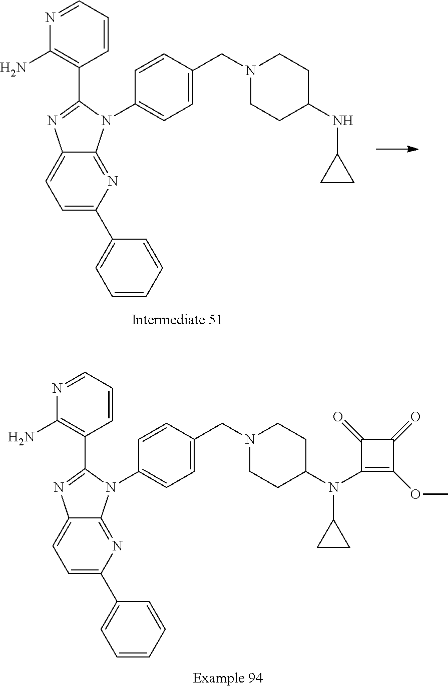 [SMILES: Nc1ncccc1-c1nc2ccc(-c3ccccc3)nc2n1-c1ccc(CN2CCC(NC3CC3)CC2)cc1 ; COc1c(N(C2CC2)C2CCN(Cc3ccc(-n4c(-c5cccnc5N)nc5ccc(-c6ccccc6)nc54)cc3)CC2)c(=O)c1=O]

To a solution of Intermediate 51 (180 mg, 349 gmol) and 3,4-dimethoxycyclobut-3-ene-1,2-dione (59.5 mg, 419 gmol) in MeOH (1 mL) was added TEA (106 mg, 1.05 mmol). The mixture was stirred at 25° C. for 16 hr. The reaction mixture was filtrated. The solid was collected, washed with MeOH (10 mL×2), dried under reduced pressure. After triturated with MeOH (20 mL), 3-((1-(4-(2-(2-aminopyridin-3-yl)-5-phenyl-3H-imidazo[4,5-b]pyridin-3-yl)benzyl)piperidin-4-yl)(cyclopropyl)amino)-4-methoxycyclobut-3-ene-1,2-dione (Example 94, 224 mg, yield: 92%) was obtained as a brown solid. MS: m/z=626.3 [M+H]⁺. ¹H NMR (400 MHz, Dimethylsulfoxide-d₆) δ 8.27 (d, J=8.4 Hz, 1H), 8.04-7.97 (m, 4H), 7.50-7.44 (m, 6H), 7.42-7.37 (m, 1H), 7.15 (dd, J=7.6, 1.6 Hz, 1H), 7.03 (br s, 2H), 6.38 (dd, J=7.6, 4.8 Hz, 1H), 4.30 (s, 3H), 3.59 (s, 2H), 3.29-3.28 (m, 1H), 2.96-2.90 (m, 2H), 2.87-2.81 (m, 1H), 2.05-1.98 (m, 4H), 1.84-1.74 (m, 2H), 0.82-0.76 (m, 4H).

### Example 95: 3-((1-(4-(2-(2-Aminopyridin-3-yl)-5-phenyl-3H-imidazo[4,5-b]pyridin-3-yl)benzyl)piperidin-4-yl)(cyclopropyl)amino)-4-(methylthio)cyclobut-3-ene-1,2-dione

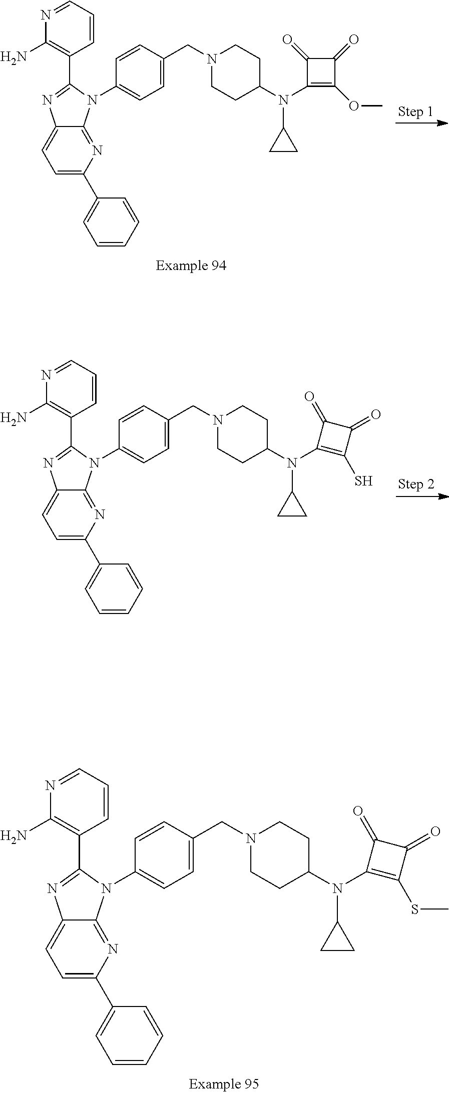 [SMILES: COc1c(N(C2CC2)C2CCN(Cc3ccc(-n4c(-c5cccnc5N)nc5ccc(-c6ccccc6)nc54)cc3)CC2)c(=O)c1=O ; Nc1ncccc1-c1nc2ccc(-c3ccccc3)nc2n1-c1ccc(CN2CCC(N(c3c(S)c(=O)c3=O)C3CC3)CC2)cc1 ; CSc1c(N(C2CC2)C2CCN(Cc3ccc(-n4c(-c5cccnc5N)nc5ccc(-c6ccccc6)nc54)cc3)CC2)c(=O)c1=O]

### Step 1: 3-((1-(4-(2-(2-Aminopyridin-3-yl)-5-phenyl-3H-imidazo[4,5-b]pyridin-3-yl)benzyl)piperidin-4-yl)(cyclopropyl)amino)-4-mercaptocyclobut-3-ene-1,2-dione

To a solution of Example 94 (100 mg, 160 gmol) in EtOH (5 mL) was added NaHS (44.8 mg, 799 μmol). The mixture was stirred at 25° C. for 14 hr. The reaction was concentrated under reduced pressure to give 3-((1-(4-(2-(2-aminopyridin-3-yl)-5-phenyl-3H-imidazo[4,5-b]pyridin-3-yl)benzyl)piperidin-4-yl)(cyclopropyl)amino)-4-mercaptocyclobut-3-ene-1,2-dione (100 mg, yield: 95%) as a yellow solid. MS: m/z=628.2 [M+H]⁺.

### Step 2: 3-((1-(4-(2-(2-Aminopyridin-3-yl)-5-phenyl-3H-imidazo[4,5-b]pyridin-3-yl)benzyl)piperidin-4-yl)(cyclopropyl)amino)-4-(methylthio)cyclobut-3-ene-1,2-dione

To a solution of 3-((1-(4-(2-(2-aminopyridin-3-yl)-5-phenyl-3H-imidazo[4,5-b]pyridin-3-yl)benzyl)piperidin-4-yl)(cyclopropyl)amino)-4-mercaptocyclobut-3-ene-1,2-dione (100 mg, 160 gmol) in EtOH (5 mL) was added CH₃I (68.1 mg, 479 gmol). The mixture was stirred at 0° C. for 4 hr. The reaction was concentrated under reduced pressure. After purified by prep-HPLC (column: Waters xbridge 150×25 mm×10 m; mobile phase: [water (NH₄HCO₃)-ACN]; gradient: 50%-80%, 10 min), 3-((1-(4-(2-(2-aminopyridin-3-yl)-5-phenyl-3H-imidazo[4,5-b]pyridin-3-yl)benzyl)piperidin-4-yl)(cyclopropyl)amino)-4-(methylthio)cyclobut-3-ene-1,2-dione (Example 95, 29.5 mg, yield: 28%) was obtained as a yellow solid. MS: m/z=642.4 [M+H]⁺. ¹H NMR (400 MHz, Methanol-d₄) δ 8.19 (d, J=8.4 Hz, 1H), 8.06-8.02 (m, 2H), 7.98 (dd, J=5.2, 2.0 Hz, 1H), 7.94 (d, J=8.4 Hz, 1H), 7.56-7.52 (m, 2H), 7.47-7.35 (m, 5H), 7.32 (dd, J=7.6, 1.6 Hz, 1H), 6.49 (dd, J=7.6, 5.2 Hz, 1H), 3.66 (s, 2H), 3.35-3.33 (m, 1H), 3.10-3.03 (m, 2H), 3.01-2.91 (m, 1H), 2.88 (s, 3H), 2.23-2.15 (m, 4H), 1.95-1.84 (m, 2H), 1.00-0.93 (m, 4H).

### Example 96: 3-((1-(4-(2-(2-Aminopyridin-3-yl)-5-phenyl-3H-imidazo[4,5-b]pyridin-3-yl)benzyl)piperidin-4-yl)(cyclopropyl)amino)-4-(ethylthio)cyclobut-3-ene-1,2-dione

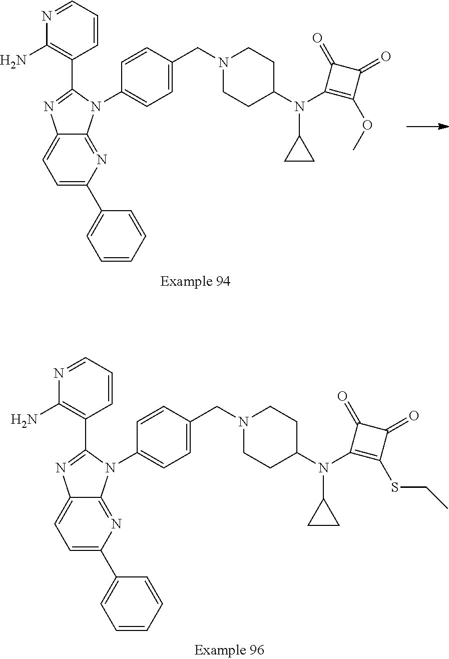 [SMILES: COc1c(N(C2CC2)C2CCN(Cc3ccc(-n4c(-c5cccnc5N)nc5ccc(-c6ccccc6)nc54)cc3)CC2)c(=O)c1=O ; CCSc1c(N(C2CC2)C2CCN(Cc3ccc(-n4c(-c5cccnc5N)nc5ccc(-c6ccccc6)nc54)cc3)CC2)c(=O)c1=O]

To a solution of Example 94 (100 mg, 160 gmol) in CH₂Cl₂ (5 mL) were added TEA (80.9 mg, 799 μmol) and ethanethiol (1.65 g, 26.6 mmol). The mixture was stirred at 25° C. for 24 hr. The reaction mixture was quenched with H₂O (5 mL) at 25° C., diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Waters xbridge 150×25 mm×10 μm; mobile phase: [water (NH₄HCO₃)-ACN]; gradient: 55%-85%, 10 min), 3-((1-(4-(2-(2-aminopyridin-3-yl)-5-phenyl-3H-imidazo[4,5-b]pyridin-3-yl)benzyl)piperidin-4-yl)(cyclopropyl)amino)-4-(ethylthio)cyclobut-3-ene-1,2-dione (Example 96, 15.7 mg, yield: 14%) was obtained as a yellow solid. MS: m/z=656.3 [M+H]⁺. ¹H NMR (400 MHz, Methanol-d₄) δ 8.19 (dd, J=8.4, 0.8 Hz, 1H), 8.06-8.01 (m, 2H), 7.98 (dd, J=4.8, 2.8 Hz, 1H), 7.94 (d, J=8.4 Hz, 1H), 7.54 (d, J=7.6 Hz, 2H), 7.47-7.30 (m, 6H), 6.49 (dd, J=8.0, 5.2 Hz, 1H), 3.66 (s, 2H), 3.47 (q, J=7.2 Hz, 2H), 3.36-3.33 (m, 1H), 3.10-2.93 (m, 3H), 2.23-2.15 (m, 4H), 1.95-1.83 (m, 2H), 1.41 (t, J=7.6 Hz, 3H), 1.02-0.94 (m, 4H).

### Example 97: 3-((4-(4-(2-(2-Aminopyridin-3-yl)-5-phenyl-3H-imidazo[4,5-b]pyridin-3-yl)benzyl)piperazin-1-yl)(methyl)amino)-4-(ethylthio)cyclobut-3-ene-1,2-dione

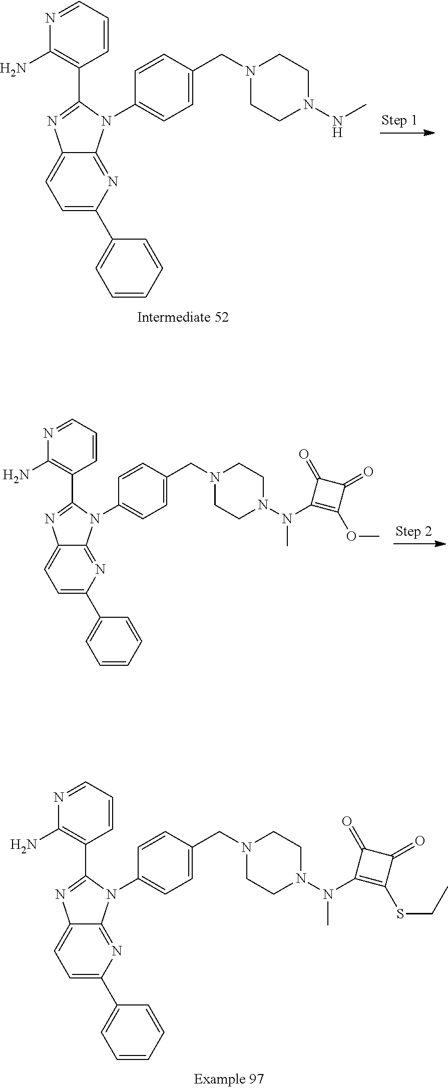 [SMILES: CNN1CCN(Cc2ccc(-n3c(-c4cccnc4N)nc4ccc(-c5ccccc5)nc43)cc2)CC1 ; COc1c(N(C)N2CCN(Cc3ccc(-n4c(-c5cccnc5N)nc5ccc(-c6ccccc6)nc54)cc3)CC2)c(=O)c1=O ; CCSc1c(N(C)N2CCN(Cc3ccc(-n4c(-c5cccnc5N)nc5ccc(-c6ccccc6)nc54)cc3)CC2)c(=O)c1=O]

### Step 1: 3-((4-(4-(2-(2-Aminopyridin-3-yl)-5-phenyl-3H-imidazo[4,5-b]pyridin-3-yl)benzyl)piperazin-1-yl)(methyl)amino)-4-methoxycyclobut-3-ene-1,2-dione

To a mixture of Intermediate 52 (150 mg, 95.0 gmol, HCl salt) and TEA (84 mg, 860 gmol) in MeOH (3 mL) was added 3,4-dimethoxycyclobut-3-ene-1,2-dione (44 mg, 312 μmol) at 0° C. The mixture was stirred at 20° C. for 12 hr under N₂. The reaction mixture was concentrated. After purified by silica gel flash chromatography (MeOH in CH₂Cl₂=0% to 6%), 3-((4-(4-(2-(2-aminopyridin-3-yl)-5-phenyl-3H-imidazo[4,5-b]pyridin-3-yl)benzyl)piperazin-1-yl)(methyl)amino)-4-methoxycyclobut-3-ene-1,2-dione (120 mg, yield: 70%) was obtained as a yellow solid. MS: m/z=601.3 [M+H]⁺.

### Step 2: 3-((4-(4-(2-(2-Aminopyridin-3-yl)-5-phenyl-3H-imidazo[4,5-b]pyridin-3-yl)benzyl)piperazin-1-yl)(methyl)amino)-4-(ethylthio)cyclobut-3-ene-1,2-dione

To a solution of 3-((4-(4-(2-(2-aminopyridin-3-yl)-5-phenyl-3H-imidazo[4,5-b]pyridin-3-yl)benzyl)piperazin-1-yl)(methyl)amino)-4-methoxycyclobut-3-ene-1,2-dione (120 mg, 200 gmol) in CH₂Cl₂ (5 mL) were added TEA (101 mg, 999 gmol) and ethanethiol (1.49 g, 24.0 mmol). The mixture was stirred at 20° C. for 12 hr. The reaction mixture was quenched with H₂O (10 mL) at 0° C. and extracted with CH₂Cl₂ (15 mL×2). The combined organic layers were washed with brine (15 mL), dried over anhydrous Na₂SO₄, filtered, and concentrated under reduced pressure. After purified by prep-HPLC (column: Boston Prime C18 150×30 mm×5 μm; mobile phase: [water (NH₃H₂O+NH₄HCO₃)-ACN]; gradient: 60%-90% B over 7 min), 3-((4-(4-(2-(2-aminopyridin-3-yl)-5-phenyl-3H-imidazo[4,5-b]pyridin-3-yl)benzyl)piperazin-1-yl)(methyl)amino)-4-(ethylthio)cyclobut-3-ene-1,2-dione (Example 97, 31.7 mg, yield: 25%) was obtained as an off white solid. MS: m/z=631.1 [M+H]⁺. ¹H NMR (400 MHz, Dimethylsulfoxide-d₆) δ 8.27 (d, J=8.4 Hz, 1H), 8.04-7.99 (m, 4H), 7.51-7.39 (m, 7H), 7.15 (d, J=6.0 Hz, 1H), 7.03 (s, 2H), 6.38 (dd, J=8.0, 5.2 Hz, 1H), 3.61 (s, 2H), 3.69-3.25 (m, 5H), 3.01-2.96 (m, 2H), 2.84-2.81 (m, 4H), 2.37-2.32 (m, 2H), 1.35 (t, J=7.2 Hz, 3H).

### Example 98: 3-((2-(4-(2-(2-Aminopyridin-3-yl)-5-phenyl-3H-imidazo[4,5-b]pyridin-3-yl)benzyl)-2-azaspiro[3.5]nonan-7-yl)amino)-4-methoxycyclobut-3-ene-1,2-dione

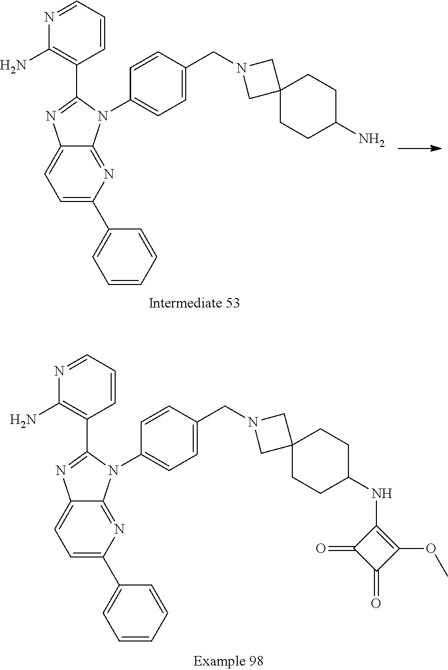 [SMILES: Nc1ncccc1-c1nc2ccc(-c3ccccc3)nc2n1-c1ccc(CN2CC3(CCC(N)CC3)C2)cc1 ; COc1c(NC2CCC3(CC2)CN(Cc2ccc(-n4c(-c5cccnc5N)nc5ccc(-c6ccccc6)nc54)cc2)C3)c(=O)c1=O]

To a solution of Intermediate 53 (100 mg, 181 gmol) and 3,4-dimethoxycyclobut-3-ene-1,2-dione (39 mg, 272 gmol) in MeOH (3 mL) was added TEA (37 mg, 362 gmol). The mixture was stirred at 25° C. for 2 hr. The reaction mixture was concentrated under reduced pressure. The residue was purified by prep-TLC (CH₂Cl₂:MeOH=10:1) to give 3-((2-(4-(2-(2-aminopyridin-3-yl)-5-phenyl-3H-imidazo[4,5-b]pyridin-3-yl)benzyl)-2-azaspiro[3.5]nonan-7-yl)amino)-4-methoxycyclobut-3-ene-1,2-dione (Example 98, 10.9 mg, yield: 9.6%, 1:1 mixture of tautomers) as a light-yellow solid. MS: m/z=626.4 [M+H]⁺. ¹H NMR (400 MHz, Methanol-d₄) δ 8.18 (d, J=8.4 Hz, 1H), 8.04-8.01 (m, 2H), 7.98 (dd, J=4.8, 1.6 Hz, 1H), 7.93 (d, J=8.4 Hz, 1H), 7.50-7.39 (m, 7H), 7.31 (dd, J=7.6, 1.6 Hz, 1H), 6.46 (dd, J=7.6, 5.2 Hz, 1H), 4.38-4.34 (m, 3H), 3.88-3.82 (m, 0.5H), 3.77 (s, 2H), 3.53-3.47 (m, 0.5H), 3.19 (s, 2H), 3.12 (s, 2H), 2.04-1.98 (m, 2H), 1.90-1.84 (m, 2H), 1.59-1.50 (m, 2H), 1.45-1.38 (m, 2H).

### Example 99: 3-(9-(4-(2-(2-Aminopyridin-3-yl)-5-phenyl-3H-imidazo[4,5-b]pyridin-3-yl)benzyl)-3,9-diazaspiro[5.5]undecan-3-yl)-4-methoxycyclobut-3-ene-1,2-dione

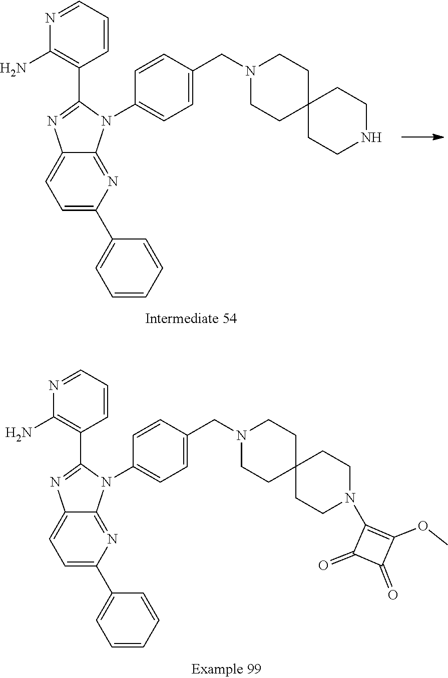 [SMILES: Nc1ncccc1-c1nc2ccc(-c3ccccc3)nc2n1-c1ccc(CN2CCC3(CCNCC3)CC2)cc1 ; COc1c(N2CCC3(CCN(Cc4ccc(-n5c(-c6cccnc6N)nc6ccc(-c7ccccc7)nc65)cc4)CC3)CC2)c(=O)c1=O]

To a solution of 3,4-dimethoxycyclobut-3-ene-1,2-dione (30.1 mg, 212 gmol) in MeOH (2 mL) were added TEA (35.8 mg, 353 μmol) and Intermediate 54 (100 mg, 177 μmol). The mixture was stirred at 25° C. for 1 hr. The mixture was added H₂O (5 mL) and extracted with EtOAc (10 mL). The organic layer was washed with brine (10 mL), dried over Na₂SO₄, filtered and concentrated under reduced pressure. The crude product was triturated with MeOH (5 mL) at 25° C. for 30 min. 3-(9-(4-(2-(2-Aminopyridin-3-yl)-5-phenyl-3H-imidazo[4,5-b]pyridin-3-yl)benzyl)-3,9-diazaspiro[5.5]undecan-3-yl)-4-methoxycyclobut-3-ene-1,2-dione (Example 99, 44.0 mg, yield: 39%) was obtained as a yellow solid. MS: m/z=640.4 [M+H]⁺. ¹H NMR (400 MHz, Methanol-d₄) δ 8.20 (d, J=8.4 Hz, 1H), 8.03 (d, J=7.2 Hz, 2H), 7.99 (dd, J=4.8, 1.6 Hz, 1H), 7.95 (d, J=8.4 Hz, 1H), 7.56 (d, J=8.0 Hz, 2H), 7.50-7.33 (m, 6H), 6.47 (dd, J=7.2, 4.8 Hz, 1H), 4.37 (s, 3H), 3.93-3.85 (m, 2H), 3.74 (s, 2H), 3.66-3.59 (m, 2H), 2.83-2.41 (m, 4H), 1.66-1.53 (m, 8H).

### Example 100: 3-((1-(4-(2-(2-Aminopyridin-3-yl)-5-phenyl-3H-imidazo[4,5-b]pyridin-3-yl)benzyl)piperidin-4-yl)(methyl-d₃)amino)-4-methoxycyclobut-3-ene-1,2-dione

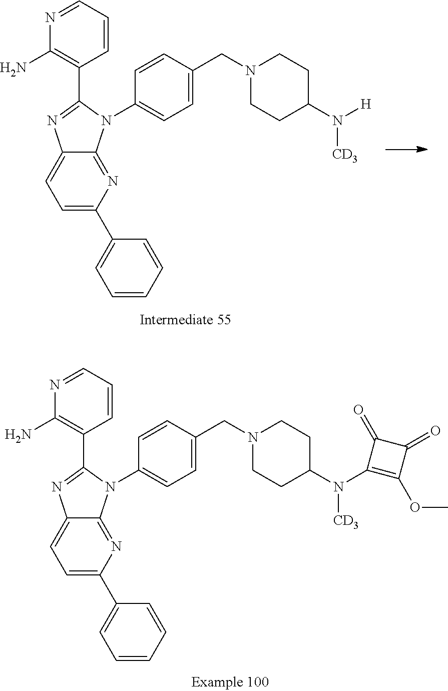 [SMILES: [2H]C([2H])([2H])NC1CCN(Cc2ccc(-n3c(-c4cccnc4N)nc4ccc(-c5ccccc5)nc43)cc2)CC1 ; [2H]C([2H])([2H])N(c1c(OC)c(=O)c1=O)C1CCN(Cc2ccc(-n3c(-c4cccnc4N)nc4ccc(-c5ccccc5)nc43)cc2)CC1]

To a solution of Intermediate 55 (100 mg, 165 gmol) in MeOH (2 mL) were added TEA (50 mg, 494 μmol) and 3,4-dimethoxycyclobut-3-ene-1,2-dione (23 mg, 165 μmol). The mixture was stirred at 25° C. for 16 hr. The mixture was filtered and the filter cake was dried under reduced pressure to give 3-((1-(4-(2-(2-aminopyridin-3-yl)-5-phenyl-3H-imidazo[4,5-b]pyridin-3-yl)benzyl)piperidin-4-yl)(methyl-d₃)amino)-4-methoxycyclobut-3-ene-1,2-dione (Example 100, 45.7 mg, yield: 44%) as a light-yellow solid. MS: m/z=603.4 [M+H]⁺, ¹H NMR (400 MHz, Dimethylsulfoxide-d₆) δ 8.27 (d, J=8.4 Hz, 1H), 8.04-7.97 (m, 4H), 7.52-7.44 (m, 6H), 7.41-7.37 (m, 1H), 7.15 (d, J=7.2 Hz, 1H), 7.02 (br s, 2H), 6.42-6.33 (m, 1H), 4.34-4.24 (m, 4H), 3.60 (s, 2H), 2.98-2.87 (m, 2H), 2.07-1.96 (m, 2H), 1.91-1.79 (m, 2H), 1.73-1.61 (m, 2H).

### II. Biological Evaluation

### Example 1: NanoBRET Target Engagement (TE) Assay

NanoBRET is a highly specific and validated cell-based technique for assessing target engagement (Vasta et al., 2018, Cell Chem Biol. 25(2):206-214). The NanoBRET™ Target Engagement (TE) Intracellular Kinase Assays are based on the NanoBRET™ System (Promega Corporation), an energy transfer technique designed to measure molecular proximity in living cells. The NanoBRET™ TE Assays measure the apparent affinity of test compounds by competitive displacement of the NanoBRET™ tracer compound, which is a cell permeable molecule engineered to be reversibly bound to a NanoLuc® luciferase-kinase fusion expressed in cells. For compound screening, when a test compound binds to the selected kinase, the BRET signal is attenuated. For kinase inhibitors in particular, intracellular target selectivity is fundamental to pharmacological mechanism and allows the proteins of interest to be in the correct cellular confirmation. Although non-cell-based techniques have been developed to measure kinase binding or enzymatic inhibition with accuracy and precision, such approaches can fail to accurately predict engagement of the full-length target protein in the more complex and biologically relevant cellular context (Knight and Shokat, 2005, Chem. Biol. 12, 621-637; Smyth and Collins, 2009, J. Chem. Biol. 2, 131-151). The NanoBRET assay procedure was used to interrogate the compounds against the full length AKT E17K per manufacturers suggestions. Briefly, HEK-293 cells (ATCC Cat #CRL-1573) were used for transfection purposes using FuGENE HD Transfection Reagent (Promega Cat #E2311). All cells were evaluated for viability prior to transfection and optimization of the transfection was done prior to experimentation. Greater than 95% viability was used for all experiments. Following transfection, cells were washed and resuspended in Opti-MEM. NanoBRET assays were performed in white, 384-well plates (Corning) at a density of 2×10⁵ cells/well. All example compounds were prepared as concentrated stock solutions in DMSO (Sigma-Aldrich). Compounds are dissolved in DMSO to make 10 mM stock solution. Example compounds were transferred as 40 μL of 10 mM stock solution to a 384 pp-plate (LABCYTE, PP-0200) and diluted in 3-fold, 10-point dilution via transferring 12 μL compound into 24 μL DMSO by Apricot liquid handler. A Labcyte ECHO 550 compound dispenser was used to facilitate compound transfer directly to cells. Cells were equilibrated for 2 hr with energy transfer probes and example compound prior to BRET measurements. The AKTE17K (Promega Cat #NV2421) as well as specific probe (NanoBRET tracer, Promega Cat #N264B) was prepared at a concentration of 20× in tracer dilution buffer (12.5 mM HEPES, 31.25% PEG-400, pH 7.5). For target engagement analysis, the energy transfer probes were added to the cells at concentrations optimized for the target in question (AKT E17K). Following compound incubation, NanoBRET NanoGlo Substrate (Promega Cat #Ni57D) and Extracellular Nanoluc Inhibitor (Promega Cat #N235C) was added according to the manufacturer's recommended protocol, and luminescence was measured on Envision Reader (Perkin Elmer) Multimode Luminometer equipped with 450 nm BPfilter (donor) and 600 nm LPfilter (acceptor), using 0.5 s integration time. Milli-BRET units (mBU) are calculated by multiplying the raw BRET values by 1000. Apparent tracer affinity values (EC50) were determined using the sigmoidal dose-response (variable slope). Competitive displacement data were then plotted and data were fit to determine the EC50 value for each example compound. Table 3 provides the assay results for select examples. Activity is defined as “+”, for EC50 greater 600 nanomolar; “++” for EC50 between 60-600 nanomolar; “+++” for EC50 between 15-60 nanomolar; and “++” for EC50 less than 15 nanomolar.

TABLE 3

| Example | | NanoBRET AKT |
|---|---|---|
| No. | Compound Name | 1E17K EC50 |
| 1 | 3-(4-(4-(2-(2-aminopyridin-3-yl)-3H-imidazo[4,5-b]pyridin-3- | + |
| | yl)benzyl)piperazin-1-yl)-4-methoxycyclobut-3-ene-1,2-dione | |
| 2 | 3-((4-(2-(2-aminopyridin-3-yl)-3H-imidazo[4,5-b]pyridin-3- | + |
| | yl)benzyl)amino)-4-methoxycyclobut-3-ene-1,2-dione | |
| 3 | 3-((1-(4-(2-(2-aminopyridin-3-yl)-3H-imidazo[4,5-b]pyridin- | +++ |
| | 3-yl)benzyl)piperidin-4-yl)amino)-4-methoxycyclobut-3-ene- | |
| | 1,2-dione | |
| 4 | 3-((4-(2-(2-aminopyridin-3-yl)-5-phenyl-3H-imidazo[4,5- | ++ |
| | b]pyridin-3-yl)benzyl)amino)-4-methoxycyclobut-3-ene-1,2- | |
| | dione | |
| 5 | 3-((4-(2-(2-aminopyridin-3-yl)-5-phenyl-3H-imidazo[4,5- | + |
| | b]pyridin-3-yl)benzyl)amino)-4-hydroxycyclobut-3-ene-1,2- | |
| | dione | |
| 6 | N-(3-(2-(2-aminopyridin-3-yl)-3-(4-(((2-methoxy-3,4- | ++ |
| | dioxocyclobut-1-en-1-yl)amino)methyl)phenyl)-3H- | |
| | imidazo[4,5-b]pyridin-5-yl)phenyl)acetamide | |
| 7 | N-(3-(2-(2-aminopyridin-3-yl)-3-(4-((4-(2-methoxy-3,4- | +++ |
| | dioxocyclobut-1-en-1-yl)piperazin-1-yl)methyl)phenyl)-3H- | |
| | imidazo[4,5-b]pyridin-5-yl)phenyl)acetamide | |
| 8 | N-(3-(2-(2-aminopyridin-3-yl)-3-(4-((4-((2-methoxy-3,4- | ++++ |
| | dioxocyclobut-1-en-1-yl)amino)piperidin-1-yl)methyl)phenyl)- | |
| | 3H-imidazo[4,5-b]pyridin-5-yl)phenyl)acetamide | |
| 9 | 3-(4-(4-(2-(2-aminopyridin-3-yl)-6-phenyl-3H-imidazo[4,5- | ++ |
| | b]pyridin-3-yl)benzyl)piperazin-1-yl)-4-methoxycyclobut-3- | |
| | ene-1,2-dione | |
| 10 | 3-((1-(4-(2-(2-aminopyridin-3-yl)-6-phenyl-3H-imidazo[4,5- | ++++ |
| | b]pyridin-3-yl)benzyl)piperidin-4-yl)amino)-4- | |
| | methoxycyclobut-3-ene-1,2-dione | |
| 11 | 3-((1-(4-(2-(2-aminopyridin-3-yl)-5-phenyl-3H-imidazo[4,5- | +++ |
| | b]pyridin-3-yl)benzyl)piperidin-4-yl)amino)-4- | |
| | isopropoxycyclobut-3-ene-1,2-dione | |
| 12 | 3-((1-(4-(2-(2-aminopyridin-3-yl)-5-phenyl-3H-imidazo[4,5- | +++ |
| | b]pyridin-3-yl)benzyl)piperidin-4-yl)amino)-4- | |
| | ethoxycyclobut-3-ene-1,2-dione | |
| 13 | 3-((1-(4-(2-(2-aminopyridin-3-yl)-5-phenyl-3H-imidazo[4,5- | ++++ |
| | b]pyridin-3-yl)benzyl)piperidin-4-yl)amino)-4-(methoxy- | |
| | d3)cyclobut-3-ene-1,2-dione | |
| 14 | 3-((1-(4-(2-(2-aminopyridin-3-yl)-5-phenyl-3H-imidazo[4,5- | ++ |
| | b]pyridin-3-yl)benzyl)piperidin-4-yl)amino)-4- | |
| | (cyclopentyloxy)cyclobut-3-ene-1,2-dione | |
| 15 | 2-((1-(4-(2-(2-aminopyridin-3-yl)-5-phenyl-3H-imidazo[4,5- | ++ |
| | b]pyridin-3-yl)benzyl)piperidin-4-yl)amino)-3- | |
| | (cyclopentyloxy)-4-thioxocyclobut-2-en-1-one | |
| 16 | 3-((1-(4-(2-(2-aminopyridin-3-yl)-5-phenyl-3H-imidazo[4,5- | ++ |
| | b]pyridin-3-yl)benzyl)piperidin-4-yl)amino)-4- | |
| | (cyclopentyloxy)cyclobut-3-ene-1,2-dithione | |
| 17 | 3-((1-(4-(2-(2-aminopyridin-3-yl)-5-phenyl-3H-imidazo[4,5- | ++++ |
| | b]pyridin-3-yl)benzyl)piperidin-4-yl)amino)-4- | |
| | methoxycyclobut-3-ene-1,2-dione | |
| 18 | 3-((1-(4-(2-(2-aminopyridin-3-yl)-5-phenyl-3H-imidazo[4,5- | + |
| | b]pyridin-3-yl)benzyl)piperidin-4-yl)amino)-4- | |
| | hydroxycyclobut-3-ene-1,2-dione | |
| 19 | 3-((1-(4-(2-(2-aminopyridin-3-yl)-5-phenyl-3H-imidazo[4,5- | + |
| | b]pyridin-3-yl)benzyl)piperidin-4-yl)amino)-4- | |
| | (dimethylamino)cyclobut-3-ene-1,2-dione | |
| 20 | 3-((1-(4-(2-(2-aminopyridin-3-yl)-5-phenyl-3H-imidazo[4,5- | ++ |
| | b]pyridin-3-yl)benzyl)piperidin-4-yl)amino)-4- | |
| | (methylamino)cyclobut-3-ene-1,2-dione | |
| 21 | 3-(4-(4-(2-(2-aminopyridin-3-yl)-5-phenyl-3H-imidazo[4,5- | +++ |
| | b]pyridin-3-yl)benzyl)piperazin-1-yl)-4-methoxycyclobut-3- | |
| | ene-1,2-dione | |
| 22 | 3-(4-(4-(2-(2-aminopyridin-3-yl)-5-phenyl-3H-imidazo[4,5- | + |
| | b]pyridin-3-yl)benzyl)piperazin-1-yl)-4-hydroxycyclobut-3- | |
| | ene-1,2-dione | |
| 23 | 3-(4-(4-(2-(2-aminopyridin-3-yl)-5-phenyl-3H-imidazo[4,5- | +++ |
| | b]pyridin-3-yl)benzyl)piperazin-1-yl)-4-(methoxy- | |
| | d3)cyclobut-3-ene-1,2-dione | |
| 24 | 3-(4-(4-(2-(2-aminopyridin-3-yl)-5-phenyl-3H-imidazo[4,5- | ++ |
| | b]pyridin-3-yl)benzyl)piperazin-1-yl)-4-ethoxycyclobut-3-ene- | |
| | 1,2-dione | |
| 25 | 3-(4-(4-(2-(2-aminopyridin-3-yl)-5-phenyl-3H-imidazo[4,5- | + |
| | b]pyridin-3-yl)benzyl)piperazin-1-yl)-4-isopropoxycyclobut-3- | |
| | ene-1,2-dione | |
| 26 | 3-(4-(4-(2-(2-aminopyridin-3-yl)-5-phenyl-3H-imidazo[4,5- | + |
| | b]pyridin-3-yl)benzyl)piperazin-1-yl)-4- | |
| | (cyclopentyloxy)cyclobut-3-ene-1,2-dione | |
| 27 | 3-((1-(4-(2-(2-aminopyridin-3-yl)-5-phenyl-3H-imidazo[4,5- | ++++ |
| | b]pyridin-3-yl)benzyl)piperidin-4-yl)(methyl)amino)-4- | |
| | methoxycyclobut-3-ene-1,2-dione | |
| 28 | 3-((1-(4-(2-(2-aminopyridin-3-yl)-5-phenyl-3H-imidazo[4,5- | + |
| | b]pyridin-3-yl)benzoyl)piperidin-4-yl)amino)-4- | |
| | methoxycyclobut-3-ene-1,2-dione | |
| 29 | (S)-3-((1-(4-(2-(2-aminopyridin-3-yl)-5-phenyl-3H- | ++ |
| | imidazo[4,5-b]pyridin-3-yl)benzyl)piperidin-3-yl)amino)-4- | |
| | methoxycyclobut-3-ene-1,2-dione | |
| 30 | (S)-3-((1-(4-(2-(2-aminopyridin-3-yl)-5-phenyl-3H- | ++++ |
| | imidazo[4,5-b]pyridin-3-yl)benzyl)pyrrolidin-3-yl)amino)-4- | |
| | methoxycyclobut-3-ene-1,2-dione | |
| 31 | 3-(9-(4-(2-(2-aminopyridin-3-yl)-5-phenyl-3H-imidazo[4,5- | +++ |
| | b]pyridin-3-yl)benzyl)-2,9-diazaspiro[5.5]undecan-2-yl)-4- | |
| | methoxycyclobut-3-ene-1,2-dione | |
| 32 | 3-((1-(4-(2-(2-aminopyridin-3-yl)-6-phenyl-3H-imidazo[4,5- | ++ |
| | b]pyridin-3-yl)benzyl)piperidin-4-yl)(methyl)amino)-4- | |
| | methoxycyclobut-3-ene-1,2-dione | |
| 33 | N-(4-(2-(2-aminopyridin-3-yl)-5-phenyl-3H-imidazo[4,5- | ++ |
| | b]pyridin-3-yl)benzyl)-3-((2-methoxy-3,4-dioxocyclobut-1-en- | |
| | 1-yl)amino)benzamide | |
| 34 | (R)-3-((1-(4-(2-(2-aminopyridin-3-yl)-5-phenyl-3H- | ++++ |
| | imidazo[4,5-b]pyridin-3-yl)benzyl)piperidin-3-yl)amino)-4- | |
| | methoxycyclobut-3-ene-1,2-dione | |
| 35 | 3-((7-(4-(2-(2-aminopyridin-3-yl)-5-phenyl-3H-imidazo[4,5- | ++++ |
| | b]pyridin-3-yl)benzyl)-7-azaspiro[3.5]nonan-2-yl)amino)-4- | |
| | methoxycyclobut-3-ene-1,2-dione | |
| 36 | (S)-3-(4-(4-(2-(2-aminopyridin-3-yl)-5-phenyl-3H- | +++ |
| | imidazo[4,5-b]pyridin-3-yl)benzyl)-2-methylpiperazin-1-yl)-4- | |
| | methoxycyclobut-3-ene-1,2-dione | |
| 37 | 3-(7-(4-(2-(2-aminopyridin-3-yl)-5-phenyl-3H-imidazo[4,5- | +++ |
| | b]pyridin-3-yl)benzyl)-4,7-diazaspiro[2.5]octan-4-yl)-4- | |
| | methoxycyclobut-3-ene-1,2-dione | |
| 38 | 3-(4-(4-(2-(2-aminopyridin-3-yl)-5-phenyl-3H-imidazo[4,5- | ++++ |
| | b]pyridin-3-yl)benzyl)-1,4-diazepan-1-yl)-4-methoxycyclobut- | |
| | 3-ene-1,2-dione | |
| 39 | 3-((1-(4-(2-(2-aminopyridin-3-yl)-5-phenyl-3H-imidazo[4,5- | ++++ |
| | b]pyridin-3-yl)benzyl)piperidin-4-yl)amino)-4- | |
| | (ethylthio)cyclobut-3-ene-1,2-dione | |
| 40 | 3-(6-(4-(2-(2-aminopyridin-3-yl)-5-phenyl-3H-imidazo[4,5- | ++++ |
| | b]pyridin-3-yl)benzyl)-2,6-diazaspiro[3.3]heptan-2-yl)-4- | |
| | ethoxycyclobut-3-ene-1,2-dione | |
| 41 | 3-(6-(4-(2-(2-aminopyridin-3-yl)-5-phenyl-3H-imidazo[4,5- | + |
| | b]pyridin-3-yl)benzyl)-2,6-diazaspiro[3.3]heptan-2-yl)-4- | |
| | methoxycyclobut-3-ene-1,2-dione | |
| 42 | 3-(2-(4-(2-(2-aminopyridin-3-yl)-5-phenyl-3H-imidazo[4,5- | +++ |
| | b]pyridin-3-yl)benzyl)-2,6-diazaspiro[3.4]octan-6-yl)-4- | |
| | methoxycyclobut-3-ene-1,2-dione | |
| 43 | N-(4-(2-(2-aminopyridin-3-yl)-5-phenyl-3H-imidazo[4,5- | + |
| | b]pyridin-3-yl)benzyl)-3-((2-hydroxy-3,4-dioxocyclobut-1-en- | |
| | 1-yl)amino)benzamide | |
| 44 | 3-((1-(4-(2-(2-aminopyridin-3-yl)-5-phenyl-3H-imidazo[4,5- | ++ |
| | b]pyridin-3-yl)benzyl)piperidin-4-yl)amino)-4- | |
| | mercaptocyclobut-3-ene-1,2-dione | |
| 45 | 3-((1-(4-(2-(2-aminopyridin-3-yl)-5-phenyl-3H-imidazo[4,5- | ++++ |
| | b]pyridin-3-yl)benzyl)piperidin-4-yl)amino)-4- | |
| | (methylthio)cyclobut-3-ene-1,2-dione | |
| 46 | 3-((1-(4-(2-(2-aminopyridin-3-yl)-5-phenyl-3H-imidazo[4,5- | + |
| | b]pyridin-3-yl)benzyl)piperidin-4-yl)(methyl)amino)-4- | |
| | mercaptocyclobut-3-ene-1,2-dione | |
| 47 | 3-((1-(4-(2-(2-aminopyridin-3-yl)-5-phenyl-3H-imidazo[4,5- | ++++ |
| | b]pyridin-3-yl)benzyl)piperidin-4-yl)(methyl)amino)-4- | |
| | (methylthio)cyclobut-3-ene-1,2-dione | |
| 48 | 3-((1-(4-(2-(2-aminopyridin-3-yl)-5-phenyl-3H-imidazo[4,5- | ++++ |
| | b]pyridin-3-yl)benzyl)piperidin-4-yl)(2-hydroxyethyl)amino)- | |
| | 4-methoxycyclobut-3-ene-1,2-dione | |
| 49 | 3-((1-(4-(2-(2-aminopyridin-3-yl)-5-phenyl-3H-imidazo[4,5- | ++++ |
| | b]pyridin-3-yl)benzyl)piperidin-4-yl)(methyl)amino)-4- | |
| | (ethylthio)cyclobut-3-ene-1,2-dione | |
| 50 | (R)-3-((1-(4-(2-(2-aminopyridin-3-yl)-5-phenyl-3H- | +++ |
| | imidazo[4,5-b]pyridin-3-yl)benzyl)pyrrolidin-3-yl)amino)-4- | |
| | methoxycyclobut-3-ene-1,2-dione | |
| 51 | 3-((1-(4-(2-(2-aminopyridin-3-yl)-5-phenyl-3H-imidazo[4,5- | ++++ |
| | b]pyridin-3-yl)benzyl)azetidin-3-yl)amino)-4- | |
| | methoxycyclobut-3-ene-1,2-dione | |
| 52 | 3-((2-(4-(2-(2-aminopyridin-3-yl)-5-phenyl-3H-imidazo[4,5- | +++ |
| | b]pyridin-3-yl)benzyl)-2-azaspiro[4.5]decan-8-yl)amino)-4- | |
| | methoxycyclobut-3-ene-1,2-dione | |
| 53 | 3-(7-(4-(2-(2-aminopyridin-3-yl)-5-phenyl-3H-imidazo[4,5- | ++++ |
| | b]pyridin-3-yl)benzyl)-2,7-diazaspiro[3.5]nonan-2-yl)-4- | |
| | methoxycyclobut-3-ene-1,2-dione | |
| 54 | 3-(8-(4-(2-(2-aminopyridin-3-yl)-5-phenyl-3H-imidazo[4,5- | +++ |
| | b]pyridin-3-yl)benzyl)-2,8-diazaspiro[4.5]decan-2-yl)-4- | |
| | methoxycyclobut-3-ene-1,2-dione | |
| 55 | 3-(6-(4-(2-(2-aminopyridin-3-yl)-5-phenyl-3H-imidazo[4,5- | ++++ |
| | b]pyridin-3-yl)benzyl)-2,6-diazaspiro[3.4]octan-2-yl)-4- | |
| | methoxycyclobut-3-ene-1,2-dione | |
| 56 | 3-(7-(4-(2-(2-aminopyridin-3-yl)-5-phenyl-3H-imidazo[4,5- | ++++ |
| | b]pyridin-3-yl)benzyl)-2,7-diazaspiro[4.4]nonan-2-yl)-4- | |
| | methoxycyclobut-3-ene-1,2-dione | |
| 57 | 3-(2-(4-(2-(2-aminopyridin-3-yl)-5-phenyl-3H-imidazo[4,5- | +++ |
| | b]pyridin-3-yl)benzyl)-2,8-diazaspiro[4.5]decan-8-yl)-4- | |
| | methoxycyclobut-3-ene-1,2-dione | |
| 58 | 3-(2-(4-(2-(2-aminopyridin-3-yl)-5-phenyl-3H-imidazo[4,5- | +++ |
| | b]pyridin-3-yl)benzyl)-2,7-diazaspiro[4.5]decan-7-yl)-4- | |
| | methoxycyclobut-3-ene-1,2-dione | |
| 59 | 3-(2-(4-(2-(2-aminopyridin-3-yl)-5-phenyl-3H-imidazo[4,5- | +++ |
| | b]pyridin-3-yl)benzyl)-2,7-diazaspiro[3.5]nonan-7-yl)-4- | |
| | ethoxycyclobut-3-ene-1,2-dione | |
| 60 | 3-(2-(4-(2-(2-aminopyridin-3-yl)-5-phenyl-3H-imidazo[4,5- | ++++ |
| | b]pyridin-3-yl)benzyl)-2,7-diazaspiro[3.5]nonan-7-yl)-4- | |
| | methoxycyclobut-3-ene-1,2-dione | |
| 61 | 3-(2-(4-(2-(2-aminopyridin-3-yl)-5-phenyl-3H-imidazo[4,5- | +++ |
| | b]pyridin-3-yl)benzyl)-2,7-diazaspiro[3.5]nonan-7-yl)-4- | |
| | (ethylthio)cyclobut-3-ene-1,2-dione | |
| 62 | (R)-3-(7-(4-(2-(2-aminopyridin-3-yl)-5-phenyl-3H- | ++++ |
| | imidazo[4,5-b]pyridin-3-yl)benzyl)-2,7-diazaspiro[4.4]nonan- | |
| | 2-yl)-4-methoxycyclobut-3-ene-1,2-dione | |
| 63 | 3-(5-(4-(2-(2-aminopyridin-3-yl)-5-phenyl-3H-imidazo[4,5- | ++++ |
| | b]pyridin-3-yl)benzyl)-2,5-diazabicyclo[2.2.2]octan-2-yl)-4- | |
| | methoxycyclobut-3-ene-1,2-dione | |
| 64 | 3-(2-(4-(2-(2-aminopyridin-3-yl)-5-phenyl-3H-imidazo[4,5- | ++++ |
| | b]pyridin-3-yl)benzyl)-2,7-diazaspiro[3.5]nonan-7-yl)-4- | |
| | (methylthio)cyclobut-3-ene-1,2-dione | |
| 65 | 3-(2-(4-(2-(2-aminopyridin-3-yl)-5-phenyl-3H-imidazo[4,5- | ++ |
| | b]pyridin-3-yl)benzyl)-2,6-diazaspiro[3.5]nonan-6-yl)-4- | |
| | methoxycyclobut-3-ene-1,2-dione | |
| 66 | (S)-3-(7-(4-(2-(2-aminopyridin-3-yl)-5-phenyl-3H- | ++++ |
| | imidazo[4,5-b]pyridin-3-yl)benzyl)-2,7-diazaspiro[4.4]nonan- | |
| | 2-yl)-4-methoxycyclobut-3-ene-1,2-dione | |
| 67 | (R)-3-(4-(4-(2-(2-aminopyridin-3-yl)-5-phenyl-3H- | ++ |
| | imidazo[4,5-b]pyridin-3-yl)benzyl)-2-methylpiperazin-1-yl)-4- | |
| | methoxycyclobut-3-ene-1,2-dione | |
| 68 | 3-((2S,6R)-4-(4-(2-(2-aminopyridin-3-yl)-5-phenyl-3H- | ++ |
| | imidazo[4,5-b]pyridin-3-yl)benzyl)-2,6-dimethylpiperazin-1- | |
| | yl)-4-methoxycyclobut-3-ene-1,2-dione | |
| 69 | 3-((2R,6R)-4-(4-(2-(2-aminopyridin-3-yl)-5-phenyl-3H- | ++ |
| | imidazo[4,5-b]pyridin-3-yl)benzyl)-2,6-dimethylpiperazin-1- | |
| | y1)-4-methoxycyclobut-3-ene-1,2-dione | |
| 70 | 3-((2S,6S)-4-(4-(2-(2-aminopyridin-3-yl)-5-phenyl-3H- | ++ |
| | imidazo[4,5-b]pyridin-3-yl)benzyl)-2,6-dimethylpiperazin-1- | |
| | yl)-4-methoxycyclobut-3-ene-1,2-dione | |
| 71 | 3-(8-(4-(2-(2-aminopyridin-3-yl)-5-phenyl-3H-imidazo[4,5- | ++++ |
| | b]pyridin-3-yl)benzyl)-3,8-diazabicyclo[3.2.1]octan-3-yl)-4- | |
| | methoxycyclobut-3-ene-1,2-dione | |
| 72 | 3-(3-(4-(2-(2-aminopyridin-3-yl)-5-phenyl-3H-imidazo[4,5- | ++ |
| | b]pyridin-3-yl)benzyl)-3,8-diazabicyclo[3.2.1]octan-8-yl)-4- | |
| | methoxycyclobut-3-ene-1,2-dione | |
| 73 | 3-((1-(4-(2-(2-aminopyridin-3-yl)-5-phenyl-3H-imidazo[4,5- | ++++ |
| | b]pyridin-3-yl)benzyl)azepan-4-yl)amino)-4-methoxycyclobut- | |
| | 3-ene-1,2-dione | |
| 74 | 3-((1-(4-(2-(2-aminopyridin-3-yl)-5-phenyl-3H-imidazo[4,5- | ++ |
| | b]pyridin-3-yl)benzyl)azepan-3-yl)amino)-4-methoxycyclobut- | |
| | 3-ene-1,2-dione | |
| 75 | 3-((1S,4S)-5-(4-(2-(2-aminopyridin-3-yl)-5-phenyl-3H- | ++++ |
| | imidazo[4,5-b]pyridin-3-yl)benzyl)-2,5- | |
| | diazabicyclo[2.2.1]heptan-2-yl)-4-methoxycyclobut-3-ene-1,2- | |
| | dione | |
| 76 | 3-((1R,4R)-5-(4-(2-(2-aminopyridin-3-yl)-5-phenyl-3H- | ++++ |
| | imidazo[4,5-b]pyridin-3-yl)benzyl)-2,5- | |
| | diazabicyclo[2.2.1]heptan-2-yl)-4-methoxycyclobut-3-ene-1,2- | |
| | dione | |
| 77 | 3-(4-((4-(2-(2-aminopyridin-3-yl)-5-phenyl-3H-imidazo[4,5- | +++ |
| | b]pyridin-3-yl)benzyl)amino)piperidin-1-yl)-4- | |
| | methoxycyclobut-3-ene-1,2-dione | |
| 78 | 3-(4-((4-(2-(2-aminopyridin-3-yl)-5-phenyl-3H-imidazo[4,5- | ++++ |
| | b]pyridin-3-yl)benzyl)oxy)piperidin-1-yl)-4-methoxycyclobut- | |
| | 3-ene-1,2-dione | |
| 79 | (R)-3-(4-(4-(2-(2-aminopyridin-3-yl)-5-phenyl-3H- | ++ |
| | imidazo[4,5-b]pyridin-3-yl)benzyl)-2- | |
| | (hydroxymethyl)piperazin-1-yl)-4-methoxycyclobut-3-ene-1,2- | |
| | dione | |
| 80 | (S)-3-(4-(4-(2-(2-aminopyridin-3-yl)-5-phenyl-3H- | ++ |
| | imidazo[4,5-b]pyridin-3-yl)benzyl)-2- | |
| | (hydroxymethyl)piperazin-1-yl)-4-methoxycyclobut-3-ene-1,2- | |
| | dione | |
| 81 | (S)-3-((1-(4-(2-(2-aminopyridin-3-yl)-5-phenyl-3H- | ++++ |
| | imidazo[4,5-b]pyridin-3-yl)benzyl)pyrrolidin-3- | |
| | yl)(methyl)amino)-4-methoxycyclobut-3-ene-1,2-dione | |
| 82 | 6-(1-(4-(2-(2-aminopyridin-3-yl)-5-phenyl-3H-imidazo[4,5- | ++ |
| | b]pyridin-3-yl)benzyl)pyrrolidin-3-yl)-2-oxa-6- | |
| | azabicyclo[5.2.0]non-1(7)-ene-8,9-dione | |
| 83 | 3-((4-(4-(2-(2-aminopyridin-3-yl)-5-phenyl-3H-imidazo[4,5- | ++++ |
| | b]pyridin-3-yl)benzyl)piperazin-1-yl)amino)-4- | |
| | methoxycyclobut-3-ene-1,2-dione | |
| 84 | 6-(1-(4-(2-(2-aminopyridin-3-yl)-5-phenyl-3H-imidazo[4,5- | + |
| | b]pyridin-3-yl)benzyl)piperidin-4-yl)-2-oxa-6- | |
| | azabicyclo[5.2.0]non-1(7)-ene-8,9-dione | |
| 85 | 6-(1-(4-(2-(2-aminopyridin-3-yl)-5-phenyl-3H-imidazo[4,5- | ++ |
| | b]pyridin-3-yl)benzyl)piperidin-4-yl)-2-thia-6- | |
| | azabicyclo[5.2.0]non-1(7)-ene-8,9-dione | |
| 86 | 3-((3aR,6aS)-5-(4-(2-(2-aminopyridin-3-yl)-5-phenyl-3H- | +++ |
| | imidazo[4,5-b]pyridin-3-yl)benzyl)hexahydropyrrolo[3,4- | |
| | c]pyrrol-2(1H)-yl)-4-methoxycyclobut-3-ene-1,2-dione | |
| 87 | 3-((3aR,6aS)-5-(4-(2-(2-aminopyridin-3-yl)-5-phenyl-3H- | ++++ |
| | imidazo[4,5-b]pyridin-3-yl)benzyl)hexahydropyrrolo[3,4- | |
| | c]pyrrol-2(1H)-yl)-4-(methylthio)cyclobut-3-ene-1,2-dione | |
| 88 | 3-((3aR,6aS)-5-(4-(2-(2-aminopyridin-3-yl)-5-phenyl-3H- | ++++ |
| | imidazo[4,5-b]pyridin-3-yl)benzyl)hexahydropyrrolo[3,4- | |
| | c]pyrrol-2(1H)-yl)-4-(ethylthio)cyclobut-3-ene-1,2-dione | |
| 89 | (S)-7-(4-(2-(2-aminopyridin-3-yl)-5-phenyl-3H-imidazo[4,5- | + |
| | b]pyridin-3-yl)benzyl)-5,5a,6,7,8,9-hexahydro-4H- | |
| | cyclobuta[b]pyrazino[1,2-d][1,4]oxazepine-1,2-dione | |
| 90 | 3-((1-(1-(4-(2-(2-aminopyridin-3-yl)-5-phenyl-3H- | ++++ |
| | imidazo[4,5-b]pyridin-3-yl)phenyl)ethyl)piperidin-4- | |
| | yl)(methyl)amino)-4-methoxycyclobut-3-ene-1,2-dione | |
| 91 | 3-((1-(4-(2-(2-aminopyridin-3-yl)-5-phenyl-3H-imidazo[4,5- | ++++ |
| | b]pyridin-3-yl)benzyl)piperidin-4-yl)(ethyl)amino)-4- | |
| | methoxycyclobut-3-ene-1,2-dione | |
| 92 | 3-((1-(4-(2-(2-aminopyridin-3-yl)-5-phenyl-3H-imidazo[4,5- | ++++ |
| | b]pyridin-3-yl)benzyl)piperidin-4-yl)(ethyl)amino)-4- | |
| | (methylthio)cyclobut-3-ene-1,2-dione | |
| 93 | 3-((1-(4-(2-(2-aminopyridin-3-yl)-5-phenyl-3H-imidazo[4,5- | +++ |
| | b]pyridin-3-yl)benzyl)piperidin-4-yl)(ethyl)amino)-4- | |
| | (ethylthio)cyclobut-3-ene-1,2-dione | |
| 94 | 3-((1-(4-(2-(2-aminopyridin-3-yl)-5-phenyl-3H-imidazo[4,5- | ++++ |
| | b]pyridin-3-yl)benzyl)piperidin-4-yl)(cyclopropyl)amino)-4- | |
| | methoxycyclobut-3-ene-1,2-dione | |
| 95 | 3-((1-(4-(2-(2-aminopyridin-3-yl)-5-phenyl-3H-imidazo[4,5- | +++ |
| | b]pyridin-3-yl)benzyl)piperidin-4-yl)(cyclopropyl)amino)-4- | |
| | (methylthio)cyclobut-3-ene-1,2-dione | |
| 96 | 3-((1-(4-(2-(2-aminopyridin-3-yl)-5-phenyl-3H-imidazo[4,5- | +++ |
| | b]pyridin-3-yl)benzyl)piperidin-4-yl)(cyclopropyl)amino)-4- | |
| | (ethylthio)cyclobut-3-ene-1,2-dione | |
| 97 | 3-((4-(4-(2-(2-aminopyridin-3-yl)-5-phenyl-3H-imidazo[4,5- | ++++ |
| | b]pyridin-3-yl)benzyl)piperazin-1-yl)(methyl)amino)-4- | |
| | (ethylthio)cyclobut-3-ene-1,2-dione | |
| 98 | 3-((2-(4-(2-(2-aminopyridin-3-yl)-5-phenyl-3H-imidazo[4,5- | ++++ |
| | b]pyridin-3-yl)benzyl)-2-azaspiro[3.5]nonan-7-yl)amino)-4- | |
| | methoxycyclobut-3-ene-1,2-dione | |
| 99 | 3-(9-(4-(2-(2-aminopyridin-3-yl)-5-phenyl-3H-imidazo[4,5- | +++ |
| | b]pyridin-3-yl)benzyl)-3,9-diazaspiro[5.5]undecan-3-yl)-4- | |
| | methoxycyclobut-3-ene-1,2-dione | |
| 100 | 3-((1-(4-(2-(2-aminopyridin-3-yl)-5-phenyl-3H-imidazo[4,5- | ++++ |
| | b]pyridin-3-yl)benzyl)piperidin-4-yl)(methyl-d3)amino)-4- | |
| | methoxycyclobut-3-ene-1,2-dione | |

### III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt or solvate thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1.-46. (canceled)

47. A compound having the structure of Formula (I), or a pharmaceutically acceptable salt or solvate thereof: 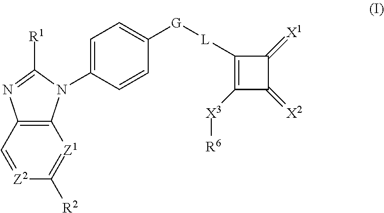
wherein:
G is O or CR⁴R⁵;
Z¹ is N, C—H, or C—R⁹;
Z² is N, C—H, or C—R³;
X¹ is O or S;
X² is O or S;
X³ is a bond, O, S, N—R⁷;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ and R⁵ are each independently hydrogen, halogen, —OH, or optionally substituted C1-C6 alkyl; or R⁴ and R⁵ together form an oxo; or R⁴ and R⁵ join together to form a carbocycle or heterocycle;
R⁶ is hydrogen, optionally substituted C1-C6 alkyl, optionally substituted C3-C7 cycloalkyl, and optionally substituted heterocyclyl; or R⁶ is absent and X³ and L join together to form a heterocycle;
R⁷ is selected from hydrogen, —OH, —NIH, optionally substituted C1-C6 alkyl, optionally substituted C3-C7 cycloalkyl, and optionally substituted heterocyclyl; or optionally, R⁶ and R⁷ join together to form a heterocycle;
L is selected from —N(R)—, or a divalent radical selected from: 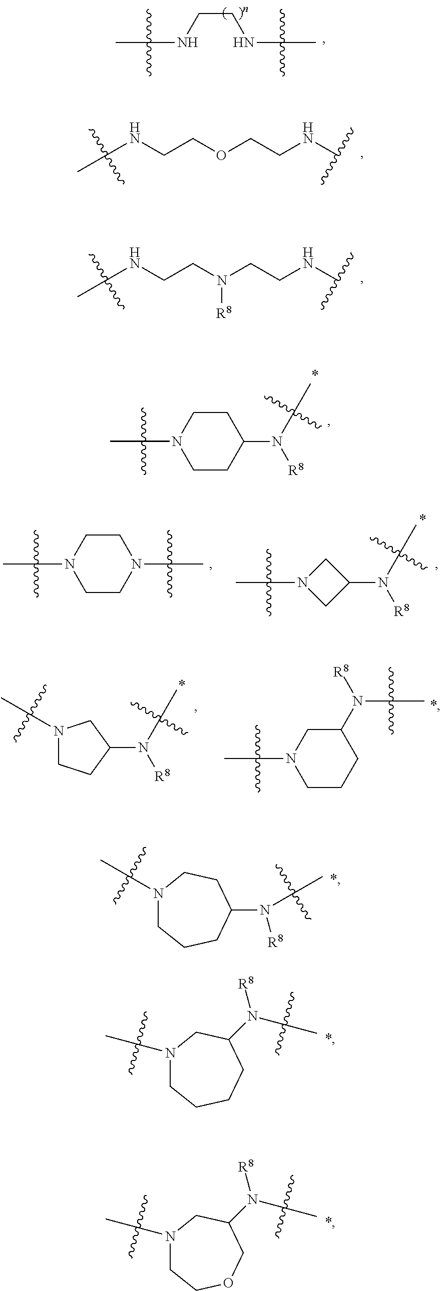 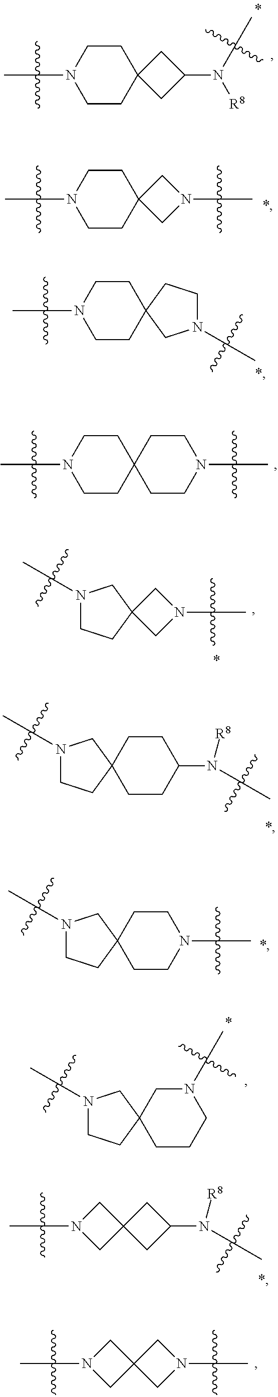 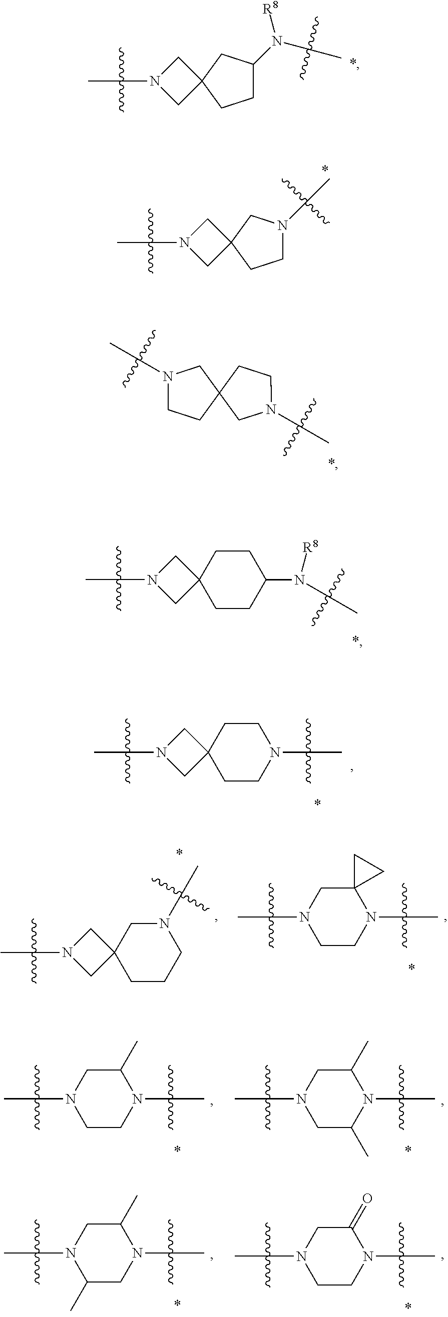 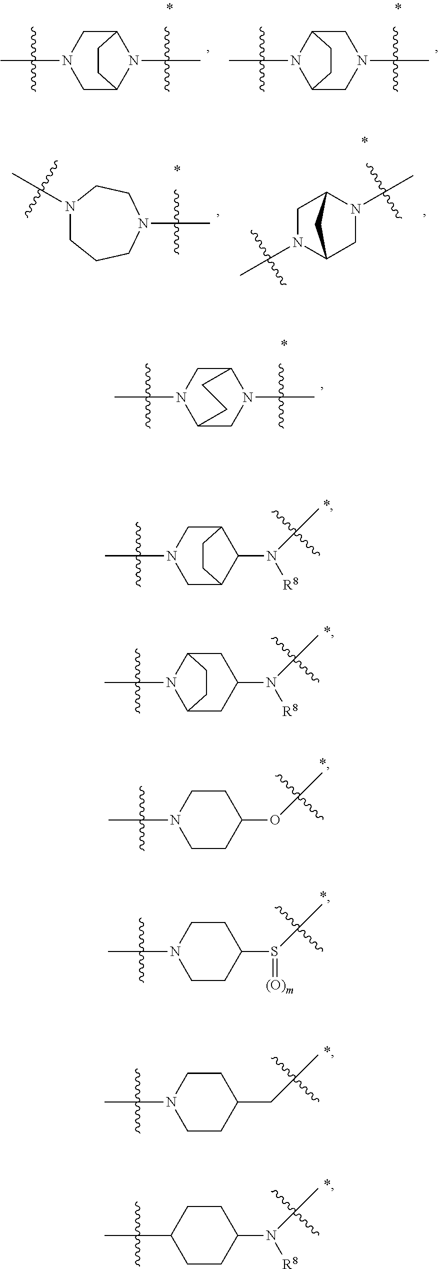 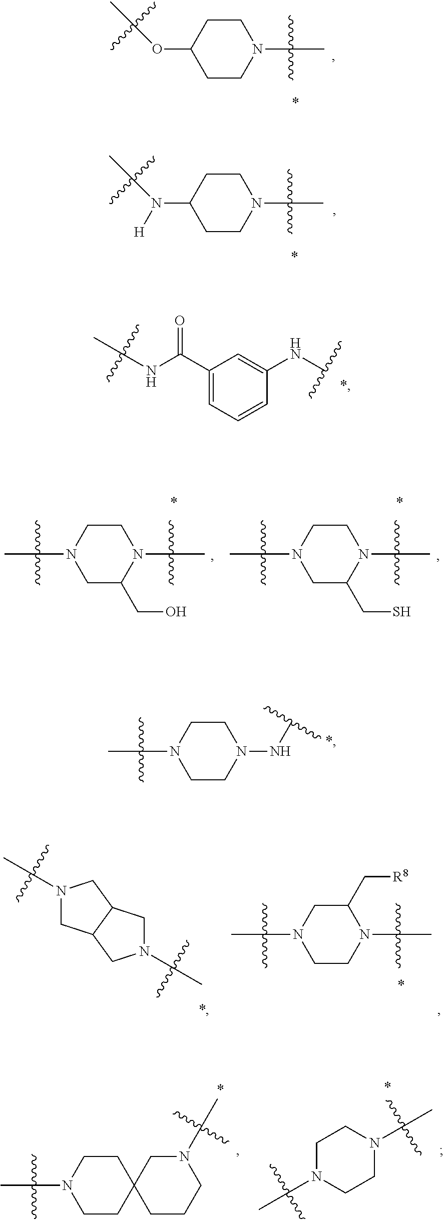
wherein the asterisk (*) indicates the bond to the squaric acid group;
R⁸ is hydrogen, optionally substituted C1-C6 alkyl, optionally substituted C3-C7 cycloalkyl, and optionally substituted heterocyclyl; or optionally R⁸ and R⁶ join to form a ring; or optionally R⁸ and R⁷ join to form a ring; and
R⁹ is selected from optionally substituted C1-C6 alkyl, or optionally substituted aryl,
m is 0, 1 or 2; and
n is 1-4.

48. The compound of claim 47, or pharmaceutically acceptable salt or solvate thereof wherein G is CR⁴R⁵.

49. The compound of claim 47, or pharmaceutically acceptable salt or solvate thereof, wherein Z is N.

50. The compound of claim 47, or pharmaceutically acceptable salt or solvate thereof, wherein Z² is C—R³.

51. The compound of claim 47, or pharmaceutically acceptable salt or solvate thereof, wherein X¹ is O.

52. The compound of claim 47, or pharmaceutically acceptable salt or solvate thereof, wherein X² is O.

53. The compound of claim 47, or pharmaceutically acceptable salt or solvate thereof, wherein X³ is O.

54. The compound of claim 47, or pharmaceutically acceptable salt or solvate thereof, wherein X³ is S.

55. The compound of claim 47, or pharmaceutically acceptable salt or solvate thereof, wherein R¹ is optionally substituted heteroaryl.

56. The compound of claim 55, or pharmaceutically acceptable salt or solvate thereof, wherein the optionally substituted heteroaryl is an optionally substituted pyridyl.

57. The compound of claim 47, or pharmaceutically acceptable salt or solvate thereof, wherein R² is optionally substituted aryl.

58. The compound of claim 57, or pharmaceutically acceptable salt or solvate thereof, wherein the optionally substituted aryl is an optionally substituted phenyl.

59. The compound of claim 47, or pharmaceutically acceptable salt or solvate thereof, wherein R⁴ is hydrogen and R⁵ is hydrogen.

60. The compound of claim 47, or pharmaceutically acceptable salt or solvate thereof, wherein R⁶ is hydrogen.

61. The compound of claim 47, or pharmaceutically acceptable salt or solvate thereof, wherein R⁶ is optionally substituted C1-C6 alkyl.

62. The compound of claim 47, or pharmaceutically acceptable salt or solvate thereof, wherein L is —N(R⁸)—.

63. The compound of claim 47, or pharmaceutically acceptable salt or solvate thereof, wherein L is selected from:

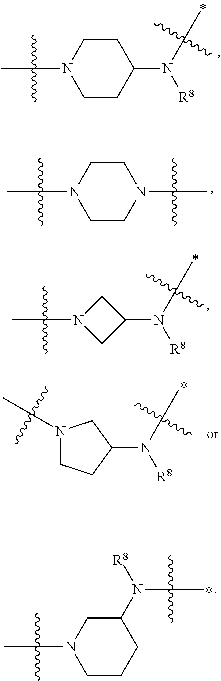

64. The compound of claim 47, or pharmaceutically acceptable salt or solvate thereof, wherein L is selected from:

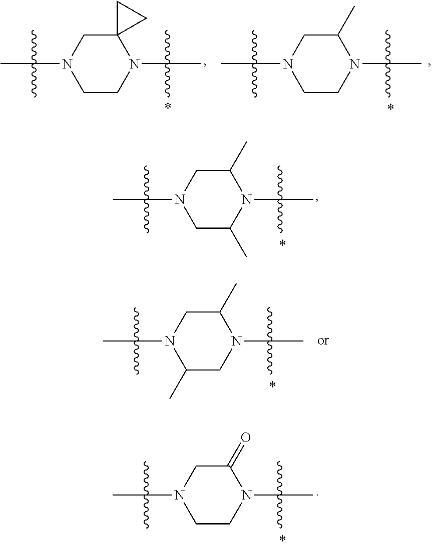

65. The compound of claim 47, or pharmaceutically acceptable salt or solvate thereof, wherein L is selected from:

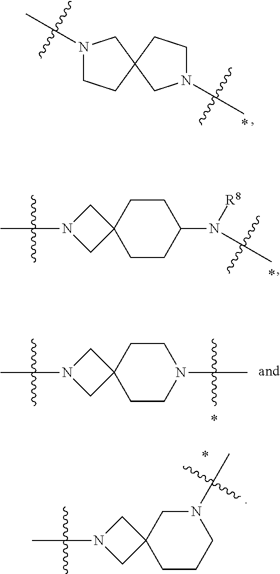

66. The compound of claim 47, or pharmaceutically acceptable salt or solvate thereof, wherein R⁸ is hydrogen or optionally substituted C1-C6 alkyl.

67. The compound of claim 47, or a pharmaceutically acceptable salt or solvate thereof, selected from the group consisting of:
3-(4-(4-(2-(2-aminopyridin-3-yl)-3H-imidazo[4,5-b]pyridin-3-yl)benzyl)piperazin-1-yl)-4-methoxycyclobut-3-ene-1,2-dione;
3-((4-(2-(2-aminopyridin-3-yl)-3H-imidazo[4,5-b]pyridin-3-yl)benzyl)amino)-4-methoxycyclobut-3-ene-1,2-dione;
3-((1-(4-(2-(2-aminopyridin-3-yl)-3H-imidazo[4,5-b]pyridin-3-yl)benzyl)piperidin-4-yl)amino)-4-methoxycyclobut-3-ene-1,2-dione;
3-((4-(2-(2-aminopyridin-3-yl)-5-phenyl-3H-imidazo[4,5-b]pyridin-3-yl)benzyl)amino)-4-methoxycyclobut-3-ene-1,2-dione;
3-((4-(2-(2-aminopyridin-3-yl)-5-phenyl-3H-imidazo[4,5-b]pyridin-3-yl)benzyl)amino)-4-hydroxy cyclobut-3-ene-1,2-dione;
N-(3-(2-(2-aminopyridin-3-yl)-3-(4-(((2-methoxy-3,4-dioxocyclobut-1-en-1-yl)amino)methyl)phenyl)-3H-imidazo[4,5-b]pyridin-5-yl)phenyl)acetamide;
N-(3-(2-(2-aminopyridin-3-yl)-3-(4-((4-(2-methoxy-3,4-dioxocyclobut-1-en-1-yl)piperazin-1-yl)methyl)phenyl)-3H-imidazo[4,5-b]pyridin-5-yl)phenyl)acetamide;
N-(3-(2-(2-aminopyridin-3-yl)-3-(4-((4-((2-methoxy-3,4-dioxocyclobut-1-en-1-yl)amino)piperidin-1-yl)methyl)phenyl)-3H-imidazo[4,5-b]pyridin-5-yl)phenyl)acetamide;
3-(4-(4-(2-(2-aminopyridin-3-yl)-6-phenyl-3H-imidazo[4,5-b]pyridin-3-yl)benzyl)piperazin-1-yl)-4-methoxycyclobut-3-ene-1,2-dione;
3-((1-(4-(2-(2-aminopyridin-3-yl)-6-phenyl-3H-imidazo[4,5-b]pyridin-3-yl)benzyl)piperidin-4-yl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)amino)-4-isopropoxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)amino)-4-(methoxy-d3)cyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)amino)-4-(cyclopentyloxy)cyclobut-3-ene-1,2-dione;
2-((1-(4-(2-(2-aminopyridin-3-yl)-5-phenyl-3H-imidazo[4,5-b]pyridin-3-yl)benzyl)piperidin-4-yl)amino)-3-(cyclopentyloxy)-4-thioxocyclobut-2-en-1-one;
3-((1-(4-(2-(2-aminopyridin-3-yl)-5-phenyl-3H-imidazo[4,5-b]pyridin-3-yl)benzyl)piperidin-4-yl)amino)-4-(cyclopentyloxy)cyclobut-3-ene-1,2-dithione;
3-((1-(4-(2-(2-aminopyridin-3-yl)-5-phenyl-3H-imidazo[4,5-b]pyridin-3-yl)benzyl)piperidin-4-yl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)amino)-4-hydroxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)amino)-4-(dimethylamino)cyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)amino)-4-(methylamino)cyclobut-3-ene-1,2-dione;
3-(4-(4-(2-(2-aminopyridin-3-yl)-5-phenyl-3H-imidazo[4,5-b]pyridin-3-yl)benzyl)piperazin-1-yl)-4-methoxycyclobut-3-ene-1,2-dione;
3-(4-(4-(2-(2-aminopyridin-3-yl)-5-phenyl-3H-imidazo[4,5-b]pyridin-3-yl)benzyl)piperazin-1-yl)-4-hydroxycyclobut-3-ene-1,2-dione;
3-(4-(4-(2-(2-aminopyridin-3-yl)-5-phenyl-3H-imidazo[4,5-b]pyridin-3-yl)benzyl)piperazin-1-yl)-4-(methoxy-d3)cyclobut-3-ene-1,2-dione;
3-(4-(4-(2-(2-aminopyridin-3-yl)-5-phenyl-3H-imidazo[4,5-b]pyridin-3-yl)benzyl)piperazin-1-yl)-4-ethoxycyclobut-3-ene-1,2-dione;
3-(4-(4-(2-(2-aminopyridin-3-yl)-5-phenyl-3H-imidazo[4,5-b]pyridin-3-yl)benzyl)piperazin-1-yl)-4-isopropoxycyclobut-3-ene-1,2-dione;
3-(4-(4-(2-(2-aminopyridin-3-yl)-5-phenyl-3H-imidazo[4,5-b]pyridin-3-yl)benzyl)piperazin-1-yl)-4-(cyclopentyloxy)cyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(methyl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzoyl)piperidin-4-yl)amino)-4-methoxycyclobut-3-ene-1,2-dione;
(S)-3-((1-(4-(2-(2-aminopyridin-3-yl)-5-phenyl-3H-imidazo[4,5-b]pyridin-3-yl)benzyl)piperidin-3-yl)amino)-4-methoxycyclobut-3-ene-1,2-dione;
(S)-3-((1-(4-(2-(2-aminopyridin-3-yl)-5-phenyl-3H-imidazo[4,5-b]pyridin-3-yl)benzyl)pyrrolidin-3-yl)amino)-4-methoxycyclobut-3-ene-1,2-dione;
3-(9-(4-(2-(2-aminopyridin-3-yl)-5-phenyl-3H-imidazo[4,5-b]pyridin-3-yl)benzyl)-2,9-diazaspiro[5.5]undecan-2-yl)-4-methoxycyclobut-3-ene-1,2-dione;
3-((1-(4-(2-(2-aminopyridin-3-yl)-6-phenyl-3H-imidazo[4,5-b]pyridin-3-yl)benzyl)piperidin-4-yl)(methyl)amino)-4-methoxycyclobut-3-ene-1,2-dione;
N-(4-(2-(2-aminopyridin-3-yl)-5-phenyl-3H-imidazo[4,5-b]pyridin-3-yl)benzyl)-3-((2-methoxy-3,4-dioxocyclobut-1-en-1-yl)amino)benzamide;
(R)-3-((1-(4-(2-(2-aminopyridin-3-yl)-5-phenyl-3H-imidazo[4,5-b]pyridin-3-yl)benzyl)piperidin-3-yl)amino)-4-methoxycyclobut-3-ene-1,2-dione;
3-((7-(4-(2-(2-aminopyridin-3-yl)-5-phenyl-3H-imidazo[4,5-b]pyridin-3-yl)benzyl)-7-azaspiro[3.5]nonan-2-yl)amino)-4-methoxycyclobut-3-ene-1,2-dione;
(S)-3-(4-(4-(2-(2-aminopyridin-3-yl)-5-phenyl-3H-imidazo[4,5-b]pyridin-3-yl)benzyl)-2-methylpiperazin-1-yl)-4-methoxycyclobut-3-ene-1,2-dione;
3-(7-(4-(2-(2-aminopyridin-3-yl)-5-phenyl-3H-imidazo[4,5-b]pyridin-3-yl)benzyl)-4,7-diazaspiro[2.5]octan-4-yl)-4-methoxycyclobut-3-ene-1,2-dione;
3-(4-(4-(2-(2-aminopyridin-3-yl)-5-phenyl-3H-imidazo[4,5-b]pyri din-3-yl)benzyl)-1,4-diazepan-1-yl)-4-methoxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)amino)-4-(ethylthio)cyclobut-3-ene-1,2-dione;
3-(6-(4-(2-(2-aminopyridin-3-yl)-5-phenyl-3H-imidazo[4,5-b]pyridin-3-yl)benzyl)-2,6-diazaspiro[3.3]heptan-2-yl)-4-ethoxycyclobut-3-ene-1,2-dione;
3-(6-(4-(2-(2-aminopyridin-3-yl)-5-phenyl-3H-imidazo[4,5-b]pyridin-3-yl)benzyl)-2,6-diazaspiro[3.3]heptan-2-yl)-4-methoxycyclobut-3-ene-1,2-dione;
3-(2-(4-(2-(2-aminopyridin-3-yl)-5-phenyl-3H-imidazo[4,5-b]pyridin-3-yl)benzyl)-2,6-diazaspiro[3.4]octan-6-yl)-4-methoxycyclobut-3-ene-1,2-dione;
N-(4-(2-(2-aminopyridin-3-yl)-5-phenyl-3H-imidazo[4,5-b]pyridin-3-yl)benzyl)-3-((2-hydroxy-3,4-dioxocyclobut-1_-en-1-yl)amino)benzamide;
3-((1-(4-(2-(2-aminopyridin-3-yl)-5-phenyl-3H-imidazo[4,5-b]pyridin-3-yl)benzyl)piperidin-4-yl)amino)-4-mercaptocyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)amino)-4-(methylthio)cyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(methyl)amino)-4-mercaptocyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(methyl)amino)-4-(methylthio)cyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(2-hydroxyethyl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(methyl)amino)-4-(ethylthio)cyclobut-3-ene-1,2-dione;
(R)-3-((1-(4-(2-(2-aminopyridin-3-yl)-5-phenyl-3H-imidazo[4,5-b]pyridin-3-yl)benzyl)pyrrolidin-3-yl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzyl)azetidin-3-yl)amino)-4-methoxycyclobut-3-ene-1,2-dione;
3-((2-(4-(2-(2-aminopyridin-3-yl)-5-phenyl-3FH-imidazo[4,5-b]pyridin-3-yl)benzyl)-2-azaspiro[4.5]decan-8-yl)amino)-4-methoxycyclobut-3-ene-1,2-dione;
3-(7-(4-(2-(2-aminopyridin-3-yl)-5-phenyl-3H-imidazo[4,5-b]pyri din-3-yl)benzyl)-2,7-diazaspiro[3.5]nonan-2-yl)-4-methoxycyclobut-3-ene-1,2-dione;
3-(8-(4-(2-(2-aminopyridin-3-yl)-5-phenyl-3JH-imidazo[4,5-b]pyridin-3-yl)benzyl)-2,8-diazaspiro[4.5]decan-2-yl)-4-methoxycyclobut-3-ene-1,2-dione;
3-(6-(4-(2-(2-aminopyridin-3-yl)-5-phenyl-3H-imidazo[4,5-b]pyridin-3-yl)benzyl)-2,6-diazaspiro[3.4]octan-2-yl)-4-methoxycyclobut-3-ene-1,2-dione;
3-(7-(4-(2-(2-aminopyridin-3-yl)-5-phenyl-3H-imidazo[4,5-b]pyridin-3-yl)benzyl)-2,7-diazaspiro[4.4]nonan-2-yl)-4-methoxycyclobut-3-ene-1,2-dione;
3-(2-(4-(2-(2-aminopyridin-3-yl)-5-phenyl-3H-imidazo[4,5-b]pyridin-3-yl)benzyl)-2,8-diazaspiro[4.5]decan-8-yl)-4-methoxycyclobut-3-ene-1,2-dione;
3-(2-(4-(2-(2-aminopyridin-3-yl)-5-phenyl-3H-imidazo[4,5-b]pyridin-3-yl)benzyl)-2,7-diazaspiro[4.5]decan-7-yl)-4-methoxycyclobut-3-ene-1,2-dione;
3-(2-(4-(2-(2-aminopyridin-3-yl)-5-phenyl-3H-imidazo[4,5-b]pyridin-3-yl)benzyl)-2,7-diazaspiro[3.5]nonan-7-yl)-4-ethoxycyclobut-3-ene-1,2-dione;
3-(2-(4-(2-(2-aminopyridin-3-yl)-5-phenyl-3H-imidazo[4,5-b]pyridin-3-yl)benzyl)-2,7-diazaspiro[3.5]nonan-7-yl)-4-methoxycyclobut-3-ene-1,2-dione;
3-(2-(4-(2-(2-aminopyridin-3-yl)-5-phenyl-3H-imidazo[4,5-b]pyridin-3-yl)benzyl)-2,7-diazaspiro[3.5]nonan-7-yl)-4-(ethylthio)cyclobut-3-ene-1,2-dione;
(R)-3-(7-(4-(2-(2-aminopyridin-3-yl)-5-phenyl-3H-imidazo[4,5-b]pyridin-3-yl)benzyl)-2,7-diazaspiro[4.4]nonan-2-yl)-4-methoxycyclobut-3-ene-1,2-dione;
3-(5-(4-(2-(2-aminopyridin-3-yl)-5-phenyl-3H-imidazo[4,5-b]pyridin-3-yl)benzyl)-2,5-diazabicyclo[2.2.2]octan-2-yl)-4-methoxycyclobut-3-ene-1,2-dione;
3-(2-(4-(2-(2-aminopyridin-3-yl)-5-phenyl-3H-imidazo[4,5-b]pyridin-3-yl)benzyl)-2,7-diazaspiro[3.5]nonan-7-yl)-4-(m ethylthio)cyclobut-3-ene-1,2-dione;
3-(2-(4-(2-(2-aminopyridin-3-yl)-5-phenyl-3H-imidazo[4,5-b]pyridin-3-yl)benzyl)-2,6-diazaspiro[3.5]nonan-6-yl)-4-methoxycyclobut-3-ene-1,2-dione;
(S)-3-(7-(4-(2-(2-aminopyridin-3-yl)-5-phenyl-3H-imidazo[4,5-b]pyridin-3-yl)benzyl)-2,7-diazaspiro[4.4]nonan-2-yl)-4-methoxycyclobut-3-ene-1,2-dione;
(R)-3-(4-(4-(2-(2-aminopyridin-3-yl)-5-phenyl-3H-imidazo[4,5-b]pyridin-3-yl)benzyl)-2-methylpiperazin-1-yl)-4-methoxycyclobut-3-ene-1,2-dione;
3-((2S,6R)-4-(4-(2-(2-aminopyridin-3-yl)-5-phenyl-31-L-imidazo[4,5-b]pyridin-3-yl)benzyl)-2,6-dimethylpiperazin-1-yl)-4-methoxycyclobut-3-ene-1,2-dione;
3-((2R,6R)-4-(4-(2-(2-aminopyridin-3-yl)-5-phenyl-31H-imidazo[4,5-b]pyridin-3-yl)benzyl)-2,6-dimethylpiperazin-1-yl)-4-methoxycyclobut-3-ene-1,2-dione;
3-((2S,6S)-4-(4-(2-(2-aminopyridin-3-yl)-5-phenyl-3H-imidazo[4,5-b]pyridin-3-yl)benzyl)-2,6-dimethylpiperazin-1-yl)-4-methoxycyclobut-3-ene-1,2-dione;
3-(8-(4-(2-(2-aminopyridin-3-yl)-5-phenyl-3H-imidazo[4,5-b]pyridin-3-yl)benzyl)-3,8-diazabicyclo[3.2.1]octan-3-yl)-4-methoxycyclobut-3-ene-1,2-dione;
3-(3-(4-(2-(2-aminopyridin-3-yl)-5-phenyl-3H-imidazo[4,5-b]pyridin-3-yl)benzyl)-3,8-diazabicyclo[3.2.1]octan-8-yl)-4-methoxycyclobut-3-ene-1,2-dione;
3-((1-(4-(2-(2-aminopyridin-3-yl)-5-phenyl-3H-imidazo[4,5-b]pyridin-3-yl)benzyl)azepan-4-yl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzyl)azepan-3-yl)amino)-4-methoxycyclobut-3-ene-1,2-dione;
3-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-4-methoxycyclobut-3-ene-1,2-dione;
3-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-4-methoxycyclobut-3-ene-1,2-dione;
3-(4-((4-(2-(2-aminopyridin-3-yl)-5-phenyl-3H-imidazo[4,5-b]pyri din-3-yl)benzyl)amino)piperidin-1-yl)-4-methoxycyclobut-3-ene-1,2-dione;
3-(4-((4-(2-(2-aminopyridin-3-yl)-5-phenyl-3H-imidazo[4,5-b]pyridin-3-yl)benzyl)oxy)piperidin-1-yl)-4-methoxycyclobut-3-ene-1,2-dione;
(R)-3-(4-(4-(2-(2-aminopyridin-3-yl)-5-phenyl-3H-imidazo[4,5-b]pyridin-3-yl)benzyl)-2-(hydroxymethyl)piperazin-1-yl)-4-methoxycyclobut-3-ene-1,2-dione;
(S)-3-(4-(4-(2-(2-aminopyridin-3-yl)-5-phenyl-3H-imidazo[4,5-b]pyridin-3-yl)benzyl)-2-(hydroxymethyl)piperazin-1-yl)-4-methoxycyclobut-3-ene-1,2-dione;
(S)-3-((1-(4-(2-(2-aminopyridin-3-yl)-5-phenyl-3H-imidazo[4,5-b]pyridin-3-yl)benzyl)pyrrolidin-3-yl)(methyl)amino)-4-methoxycyclobut-3-ene-1,2-di one;
6-(1-(4-(2-(2-aminopyridin-3-vi)-5-phenyl-3H-imidazo[4,5-b]pyri din-3-yl)benzyl)pyrrolidin-3-yl)-2-oxa-6-azabicyclo[5.2.0]non-1(7)-ene-8,9-dione;
3-((4-(4-(2-(2-aminopyridin-3-yl)-5-phenyl-3H-imidazo[4,5-b]pyri din-3-yl)benzyl)piperazin-1-yl)amino)-4-methoxycyclobut-3-ene-1,2-dione;
6-(1-(4-(2-(2-aminopyridin-3-yl)-5-phenyl-3H-imidazo[4,5-b]pyridin-3-yl)benzyl)piperidin-4-yl)-2-oxa-6-azabicyclo[5.2.0]non-1(7)-ene-8,9-dione;
6-(1-(4-(2-(2-aminopyridin-3-yl)-5-phenyl-3H-imidazo[4,5-b]pyridin-3-yl)benzyl)piperidin-4-yl)-2-thia-6-azabicyclo[5.2.0]non-1(7)-ene-8,9-dione;
3-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-4-methoxycyclobut-3-ene-1,2-dione;
3-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-4-(methylthio)cyclobut-3-ene-1,2-dione;
3-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-4-(ethylthio)cyclobut-3-ene-1,2-dione;
(S)-7-(4-(2-(2-aminopyridin-3-yl)-5-phenyl-3H-imidazo[4,5-b]pyri din-3-yl)benzyl)-5,5a,6,7,8,9-hexahydro-4H-cyclobuta[b]pyrazino[1,2-d][1,4]oxazepine-1,2-dione;
3-((1-(1-(4-(2-(2-aminopyridin-3-yl)-5-phenyl-31H-imidazo[4,5-b]pyridin-3-yl)phenyl)ethyl)piperidin-4-yl)(methyl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(ethyl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(ethyl)amino)-4-(methylthio)cyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(ethyl)amino)-4-(ethylthio)cyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(cyclopropyl)amino)-4-methoxycyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(cyclopropyl)amino)-4-(methylthio)cyclobut-3-ene-1,2-dione;
3-((1-(4-(2-(2-aminopyridin-3-yl)-5-phenyl-3H-imidazo[4,5-b]pyridin-3-yl)benzyl)piperidin-4-yl)(cyclopropyl)amino)-4-(ethylthio)cyclobut-3-ene-1,2-dione;
3-((4-(4-(2-(2-aminopyridin-3-yl)-5-phenyl-3H-imidazo[4,5-b]pyridin-3-yl)benzyl)piperazin-1-yl)(methyl)amino)-4-(ethylthio)cyclobut-3-ene-1,2-dione;
3-((2-(4-(2-(2-aminopyridin-3-yl)-5-phenyl-3H-imidazo[4,5-b]pyridin-3-yl)benzyl)-2-azaspiro[3.5]nonan-7-yl)amino)-4-methoxycyclobut-3-ene-1,2-dione;
3-(9-(4-(2-(2-aminopyridin-3-yl)-5-phenyl-31-imidazo[4,5-b]pyridin-3-yl)benzyl)-3,9-diazaspiro[5.5]undecan-3-yl)-4-methoxycyclobut-3-ene-1,2-dione; and
3-((1-(4-(2-(2-aminopyridin-3-yl)-5-phenyl-3H-imidazo[4,5-b]pyridin-3-yl)benzyl)piperidin-4-yl)(methyl-d3)amino)-4-methoxycyclobut-3-ene-1,2-dione.

68. A pharmaceutical composition comprising a compound, or pharmaceutically acceptable salt or solvate thereof, as described in claim 47, and a pharmaceutically acceptable excipient.

69. A method of treating cancer in a patient in need thereof, comprising administering to the patient a compound as described in claim 47, or pharmaceutically acceptable salt or solvate thereof.
